(19) United States
(12) Patent Application Publication
(10) Pub. No.: US 2023/0092163 A1
(43) Pub. Date: Mar. 23, 2023

(54) COMPOUNDS AND COMPOSITIONS FOR TREATING CONDITIONS ASSOCIATED WITH STING ACTIVITY

(71) Applicant: IFM Due, Inc., Boston, MA (US)

(72) Inventors: Jason Katz, Newton, MA (US); Shankar Venkatraman, Lansdale, PA (US); William R. Roush, Boston, MA (US)

(21) Appl. No.: 17/618,345

(22) PCT Filed: Jun. 12, 2020
(86) PCT No.: PCT/US2020/037403

§ 371 Date: Dec. 10, 2021

Related U.S. Application Data
(60) Provisional application No. 62/861,714, filed on Jun. 14, 2019.
(60) Provisional application No. 62/955,924, filed on Dec. 31, 2019.

(51) Int. Cl. C07D 401/12; C07D 209/08; C07D 209/42; C07D 471/04; C07D 401/14; C07D 403/12; C07D 403/14; C07D 487/04; C07D 405/12; C07D 491/052; C07D 401/04; C07D 403/04; C07D 417/12; C07D 498/04

(52) U.S. Cl. CPC C07D 401/12; C07D 209/08; C07D 209/42; C07D 471/04; C07D 401/14; C07D 403/12; C07D 403/14; C07D 487/04; C07D 405/12; C07D 491/052; C07D 401/04; C07D 403/04; C07D 417/12; C07D 498/04

(57) ABSTRACT

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., (cancer) in a subject (e.g., a human). This disclosure also features compositions containing the same as well as methods of using and making the same.

### CROSS REFERENCE TO RELATED APPLICATIONS

This application claims the benefit of U.S. Provisional Application Ser. No. 62/861,714, filed on Jun. 14, 2019; and U.S. Provisional Application Ser. No. 62/955,924, filed on Dec. 31, 2019; each of which is incorporated herein by reference in its entirety.

### TECHNICAL FIELD

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

### BACKGROUND

STING, also known as transmembrane protein 173 (TMEM173) and MPYS/MITA/ERIS, is a protein that in humans is encoded by the TMEM173 gene. STING has been shown to play a role in innate immunity. STING induces type I interferon production when cells are infected with intracellular pathogens, such as viruses, mycobacteria and intracellular parasites. Type I interferon, mediated by STING, protects infected cells and nearby cells from local infection in an autocrine and paracrine manner.

The STING pathway is pivotal in mediating the recognition of cytosolic DNA. In this context, STING, a transmembrane protein localized to the endoplasmic reticulum (ER), acts as a second messenger receptor for 2′, 3′ cyclic GMP-AMP (hereafter cGAMP), which is produced by cGAS after dsDNA binding. In addition, STING can also function as a primary pattern recognition receptor for bacterial cyclic dinucleotides (CDNs) and small molecule agonists. The recognition of endogenous or prokaryotic CDNs proceeds through the carboxy-terminal domain of STING, which faces into the cytosol and creates a V-shaped binding pocket formed by a STING homodimer. Ligand-induced activation of STING triggers its re-localization to the Golgi, a process essential to promote the interaction of STING with TBK1. This protein complex, in turn, signals through the transcription factors IRF-3 to induce type I interferons (IFNs) and other co-regulated antiviral factors. In addition, STING was shown to trigger NF-κB and MAP kinase activation. Following the initiation of signal transduction, STING is rapidly degraded, a step considered important in terminating the inflammatory response.

Excessive activation of STING is associated with a subset of monogenic autoinflammatory conditions, the so-called type I interferonopathies. Examples of these diseases include a clinical syndrome referred to as STING-associated vasculopathy with onset in infancy (SAVI), which is caused by gain-of-function mutations in TMEM173 (the gene name of STING). Moreover, STING is implicated in the pathogenesis of Aicardi-Goutières Syndrome (AGS) and genetic forms of lupus. As opposed to SAVI, it is the dysregulation of nucleic acid metabolism that underlies continuous innate immune activation in AGS. Apart from these genetic disorders, emerging evidence points to a more general pathogenic role for STING in a range of inflammation-associated disorders such as systemic lupus erythematosus, rheumatoid arthritis and cancer. Thus, small molecule-based pharmacological interventions into the STING signaling pathway hold significant potential for the treatment of a wide spectrum of diseases

### SUMMARY

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

An “antagonist” of STING includes compounds that, at the protein level, directly bind or modify STING such that an activity of STING is decreased, e.g., by inhibition, blocking or dampening agonist-mediated responses, altered distribution, or otherwise. STING antagonists include chemical entities, which interfere or inhibit STING signaling.

In one aspect, compounds of Formula (I), or a pharmaceutically acceptable salt thereof, are featured:

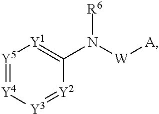

in which Y¹, Y², Y³, Y⁴, Y⁵, R⁶, W, and A can be as defined anywhere herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inhibiting (e.g., antagonizing) STING activity are featured that include contacting STING with a chemical entity described herein (e.g., a compound described generically or specifically herein or a pharmaceutically acceptable salt thereof or compositions containing the same). Methods include in vitro methods, e.g., contacting a sample that includes one or more cells comprising STING (e.g., innate immune cells, e.g., mast cells, macrophages, dendritic cells (DCs), and natural killer cells) with the chemical entity. Methods can also include in vivo methods; e.g., administering the chemical entity to a subject (e.g., a human) having a disease in which increased (e.g., excessive) STING signaling contributes to the pathology and/or symptoms and/or progression of the disease.

In one aspect, methods of treating a condition, disease or disorder ameliorated by antagonizing STING are featured, e.g., treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). The methods include administering to a subject in need of such treatment an effective amount of a chemical entity described herein (e.g., a compound described generically or specifically herein or a pharmaceutically acceptable salt thereof or compositions containing the same).

In another aspect, methods of treating cancer are featured that include administering to a subject in need of such treatment an effective amount of a chemical entity described herein (e.g., a compound described generically or specifically herein or a pharmaceutically acceptable salt thereof or compositions containing the same).

In a further aspect, methods of treating other STING-associated conditions are featured, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. The methods include administering to a subject in need of such treatment an effective amount of a chemical entity described herein (e.g., a compound described generically or specifically herein or a pharmaceutically acceptable salt thereof or compositions containing the same).

In another aspect, methods of suppressing STING-dependent type I interferon production in a subject in need thereof are featured that include administering to the subject an effective amount of a chemical entity described herein (e.g., a compound described generically or specifically herein or a pharmaceutically acceptable salt thereof or compositions containing the same).

In a further aspect, methods of treating a disease in which increased (e.g., excessive) STING activation (e.g., STING signaling) contributes to the pathology and/or symptoms and/or progression of the disease are featured. The methods include administering to a subject in need of such treatment an effective amount of a chemical entity described herein (e.g., a compound described generically or specifically herein or a pharmaceutically acceptable salt thereof or compositions containing the same).

In another aspect, methods of treatment are featured that include administering an effective amount of a chemical entity described herein (e.g., a compound described generically or specifically herein or a pharmaceutically acceptable salt thereof or compositions containing the same) to a subject; wherein the subject has (or is predisposed to have) a disease in which increased (e.g., excessive) STING activation (e.g., STING signaling) contributes to the pathology and/or symptoms and/or progression of the disease.

In a further aspect, methods of treatment that include administering to a subject a chemical entity described herein (e.g., a compound described generically or specifically herein or a pharmaceutically acceptable salt thereof or compositions containing the same), wherein the chemical entity is administered in an amount effective to treat a disease in which increased (e.g., excessive) STING activation (e.g., STING signaling) contributes to the pathology and/or symptoms and/or progression of the disease, thereby treating the disease.

Embodiments can include one or more of the following features.

The chemical entity can be administered in combination with one or more additional therapeutic agents and/or regimens. For examples, methods can further include administering one or more (e.g., two, three, four, five, six, or more) additional agents.

The chemical entity can be administered in combination with one or more additional therapeutic agents and/or regimens that are useful for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis.

The chemical entity can be administered in combination with one or more additional cancer therapies (e.g., surgery, radiotherapy, chemotherapy, toxin therapy, immunotherapy, cryotherapy or gene therapy, or a combination thereof, e.g., chemotherapy that includes administering one or more (e.g., two, three, four, five, six, or more) additional chemotherapeutic agents. Non-limiting examples of additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155 (e.g., CTLA-4 or PD1 or PD-L1).

The subject can have cancer; e.g., the subject has undergone and/or is undergoing and/or will undergo one or more cancer therapies.

Non-limiting examples of cancer include melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma. In certain embodiments, the cancer can be a refractory cancer.

The chemical entity can be administered intratumorally.

The methods can further include identifying the subject.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

As used herein, the term “STING” is meant to include, without limitation, nucleic acids, polynucleotides, oligonucleotides, sense and antisense polynucleotide strands, complementary sequences, peptides, polypeptides, proteins, homologous and/or orthologous STING molecules, isoforms, precursors, mutants, variants, derivatives, splice variants, alleles, different species, and active fragments thereof.

The term “acceptable” with respect to a formulation, composition or ingredient, as used herein, means having no persistent detrimental effect on the general health of the subject being treated.

“ApI” refers to an active pharmaceutical ingredient.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. The “treatment of cancer”, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

The term “halo” refers to fluoro (F), chloro (C1), bromo (Br), or iodo (I).

The term “alkyl” refers to a hydrocarbon chain that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—).

The term “alkenyl” refers to a hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein includes cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

The term “cycloalkenyl” as used herein includes partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. Cycloalkenyl groups may have any degree of saturation provided that none of the rings in the ring system are aromatic; and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; and having 6, 10, or 14 pi electrons shared in a cyclic array; wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl.

The term “heterocyclyl” refers to a mon-, bi-, tri-, or polycyclic nonaromatic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

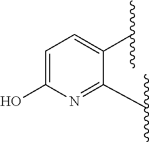 [SMILES: *c1ccc(O)nc1*]

encompasses the tautomeric form containing the moiety:

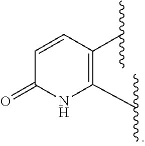 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages of the invention will be apparent from the description and drawings, and from the claims.

### DETAILED DESCRIPTION

This disclosure features chemical entities (e.g., a compound or a pharmaceutically acceptable salt, and/or hydrate, and/or cocrystal, and/or drug combination of the compound) that inhibit (e.g., antagonize) Stimulator of Interferon Genes (STING). Said chemical entities are useful, e.g., for treating a condition, disease or disorder in which increased (e.g., excessive) STING activation (e.g., STING signaling) contributes to the pathology and/or symptoms and/or progression of the condition, disease or disorder (e.g., cancer) in a subject (e.g., a human). This disclosure also features compositions containing the same as well as methods of using and making the same.

Formula I Compounds

In one aspect, provided herein is a compound of Formula I:

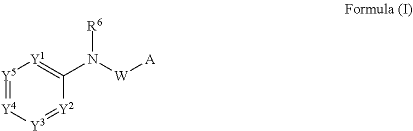

or a pharmaceutically acceptable salt thereof or a tautomer thereof,

wherein:

each of Y¹, Y², Y³, Y⁴, and Y⁵ is independently selected from the group consisting of N and CR¹;

W-A is defined according to (A) or (B) below:
(A)

W is selected from the group consisting of:
(a) *C(═O)NR^{N}, *C(═S)NR^{N}, *C(═NR^{N})NR^{N} (e.g., *C(═NCN)NR^{N}), *C(CNO₂)NR^{N}
(b) *S(O)₁₋₂NR^{N};

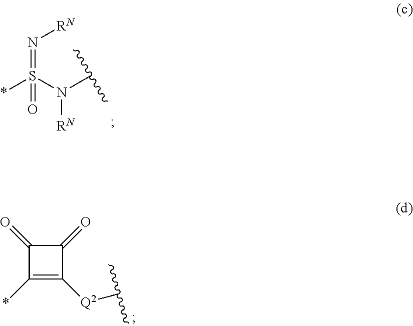

(e) *Q¹-Q²;
wherein the asterisk denotes point of attachment to NR⁶;

Q¹ is selected from the group consisting of:
(a) phenylene optionally substituted with from 1-2 independently selected R^{q1}; and
(b) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};
   Q² is selected from the group consisting of: a bond, NR^{N}, —S(O)₀₋₂—, —O—, and —C(═O)—;

### A is:

(i) —Y^{A1}-Y^{A1}, wherein:
Y^{A1} is a bond; or
Y^{A1} is C₁₋₆ alkylene, which is optionally substituted with from 1-6 substituents each independently selected from the group consisting of:
   R^{a};
   C₆₋₁₀ aryl optionally substituted with 1-4 independently selected C₁₋₄ alkyl; and
   heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; or
Y^{A1} is —Y^{A3}-Y^{A4}—Y^{A5} which is connected to W via Y^{A3} wherein:
   Y^{A3} is a C₁₋₃ alkylene optionally substituted with from 1-2 independently selected R^{a};
   Y^{A4} is —O—, —NH—, or —S—; and
   Y^{A5} is a bond or C₁₋₃ alkylene which is optionally substituted with from 1-2 independently selected R^{a}; and
Y^{A2} is:
(a) C₃₋₂₀ cycloalkyl, which is optionally substituted with from 1-4 R^{b},
(b) C₆₋₂₀ aryl, which is optionally substituted with from 1-4 R^{c};
(c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) —Z¹-Z²-Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₂₀ alkyl, which is optionally substituted with from 1-6 independently selected R^{a},

### OR

(B)
   W is selected from the group consisting of:

(a) C₈₋₂₀ bicyclic or polycyclic arylene, which is optionally substituted with from 1-4 R^{c}; and

(b) bicyclic or polycyclic heteroarylene including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

A is as defined for (A), or A is H;

each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″), and
—L³-L⁴-L⁵-Rⁱ;

or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;

each R² is independently selected from the group consisting of:
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl) optionally substituted with from 1-3 independently selected R^{a},
—S(O)(═NH)(C₁₋₄ alkyl) optionally substituted with from 1-3 independently selected R^{a},
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl) optionally substituted with from 1-3 independently selected R^{a},
—C(═O)O(C₁₋₄ alkyl) optionally substituted with from 1-3 independently selected R^{a},
—C(═O)OH,
—C(═O)N(R′)(R″); and
—L³-L⁴-L⁵-R¹;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{q1} is independently selected from the group consisting of:
(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;

each occurrence of R^{a} is independently selected from the group consisting of: —OH; —F; —Cl ; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —OCON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl ; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl) or —S(O)₁₋₂(C₁₋₄ haloalkyl); (j) —NR^{e}R^{f}; (k) —OH; (l) —S(O)₁₋₂(NR′R″); (m) —C₁₋₄ thioalkoxy or —C₁₋₄ thiohaloalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); (s) -L¹-L²-R^{h}; (t) —SF₅; and (u) azido;

each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;

each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene optionally substituted with oxo;
-L² is —O—, —N(H)—, —S(O)₀₋₂—, or a bond;
R^{h} is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
   -L³ is a bond or C₁₋₃ alkylene optionally substituted with oxo;
   -L⁴ is a bond; —O—; —N(R^{N})—; —S(O)₀₋₂—; C(═O); —NR^{N}S(O)₀₋₂—; —S(O)₀₋₂NR^{N}—; —NR^{N}S(O)₁₋₂NR^{N}—; —S(═O)(═NR^{N}); —NR^{N}S(═O)(═NR^{N}); —S(═O)(═NR^{N})NR^{N}; NR^{N}S(═O)(═NR^{N})NR^{N}; —NR^{N}C(O)—; —NR^{N}C(O)NR^{N}—; C₃₋₆ cycloalkylene; or heterocyclylene including from 3-8 ring atoms wherein from 1-3 ring atoms are heteroatoms each independently selected from the group consisting of N, NH, N(R^{d}), O, and S(O)₀₋₂;
   -L⁵ is a bond or C₁₋₄ alkylene;
   Rⁱ is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when Rⁱ is C₃₋₆ cycloalkyl optionally substituted with from 1-4 substituents independently selected C₁₋₄ alkyl, -L is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;

each occurrence of R^{N} is independently H or R^{d}; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₄ alkyl), O, and S;

In some embodiments, it is provided that one or more of the compound provisions herein apply.

In one aspect, compounds of Formula (I), or a pharmaceutically acceptable salt thereof, are featured:

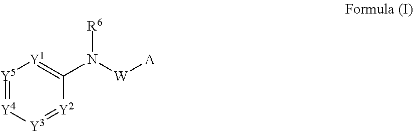

or a pharmaceutically acceptable salt thereof or a tautomer thereof,
wherein:
each of Y¹, Y², Y³, Y⁴, and Y⁵ is independently selected from the group consisting of N and CR¹;
W-A is defined according to (A) or (B) below:
W is selected from the group consisting of:
(a) *C(═O)NR^{N}, *C(═S)NR^{N}, *C(═NR^{N})NR^{N} (e.g., *C(═NCN)NR^{N}), *C(CNO₂)NR^{N}
(b) *S(O)₁₋₂NR^{N};

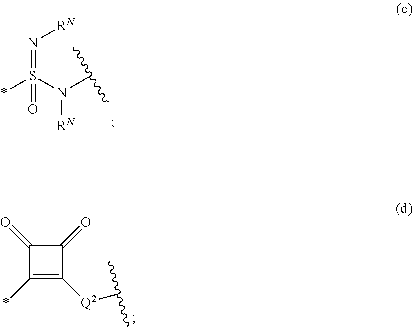

(e) *Q¹-Q²;
wherein the asterisk denotes point of attachment to NR⁶;
   Q¹ is selected from the group consisting of:
(a) phenylene optionally substituted with from 1-2 independently selected R^{q1}; and
(b) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};
   Q² is selected from the group consisting of: a bond, NR^{N}, —S(O)₀₋₂—, —O—, and —C(═O)—;

### A is:

(i) —Y^{A1}-Y^{A2}, wherein:
   Y^{A1} is a bond; or
   Y^{A1} is C₁₋₆ alkylene, which is optionally substituted with from 1-6 substituents each independently selected from the group consisting of:
      R^{a};
      C₆₋₁₀ aryl optionally substituted with 1-4 independently selected C₁₋₄ alkyl; and
      heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; or
   Y^{A1} is —Y^{A3}—Y^{A4}-Y^{A5} which is connected to W via Y^{A3} wherein:
      Y^{A3} is a C₁₋₃ alkylene optionally substituted with from 1-2 independently selected R^{a};
      Y^{A4} is —O—, —NH—, or —S—; and
      Y^{A5} is a bond or C₁₋₃ alkylene which is optionally substituted with from 1-2 independently selected R^{a}; and
   Y^{A2} is:
(a) C₃₋₂₀ cycloalkyl, which is optionally substituted with from 1-4 R^{b},
(b) C₆₋₂₀ aryl, which is optionally substituted with from 1-4 R^{c};
(c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) —Z¹-Z²-Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₂₀ alkyl, which is optionally substituted with from 1-6 independently selected R^{a},

### OR

(B)
   W is selected from the group consisting of:
(a) C₈₋₂₀ bicyclic or polycyclic arylene, which is optionally substituted with from 1-4 R^{c}; and
(b) bicyclic or polycyclic heteroarylene including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

A is as defined for (A), or A is H;

each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)O
—C(═O)N(R′)(R″), and
—L³-L⁴-L⁵-Rⁱ;

or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;

each R² is independently selected from the group consisting of: halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl) optionally substituted with from 1-3 independently selected R^{a}; —S(O)(═NH)(C₁₋₄ alkyl) optionally substituted with from 1-3 independently selected R^{a}; SF₅; —NR^{e}R^{f}; —OH; oxo; —S(O)₁₋₂(NR′R″); —C₁₋₄ thioalkoxy; —NO₂; —C(═O)(C₁₋₄ alkyl) optionally substituted with from 1-3 independently selected R^{a}; —C(═O)O(C₁₋₄ alkyl) optionally substituted with from 1-3 independently selected R^{a}; —C(═O)OH; —C(═O)N(R′)(R″); and -L³-L⁴-L⁵-R¹;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{q1} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;

each occurrence of R^{a} is independently selected from the group consisting of: —OH; —F; —Cl ; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl ; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:
(a) halo;
(b) cyano;
(c) C₁₋₁₀ alkyl which is optionally substituted with from 1-6 independently selected R^{a};
(d) C₂₋₆ alkenyl;
(e) C₂₋₆ alkynyl;
(g) C₁₋₄ alkoxy;
(h) C₁₋₄ haloalkoxy;
(i) —S(O)₁₋₂(C₁₋₄ alkyl) or —S(O)₁₋₂(C₁₋₄ haloalkyl);
(j) —NR^{e}R_{f};
(k) —OH;
(l) —S(O)₁₋₂(NR′R″);
(m) —C₁₋₄ thioalkoxy or —C₁₋₄ thiohaloalkoxy;
(n) —NO₂;
(o) —C(═O)(C₁₋₁₀ alkyl);
(p) —C(═O)O(C₁₋₄ alkyl);
(q) —C(═O)OH;
(r) —C(═O)N(R′)(R″);
(s) -L¹-L²-R^{h}; and
(t) —SF₅
   each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;
   each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁. 6 haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;
   -L¹ is a bond or C₁₋₃ alkylene optionally substituted with oxo;
   -L² is —O—, —N(H)—, —S(O)₀₋₂—, or a bond;
   R^{h} is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
   -L³ is a bond or C₁₋₃ alkylene optionally substituted with oxo;
   —L⁴ is —O—, —N(R^{N})—, —S(O)₀₋₂—, C(═O), —NR^{N}S(O)₀₋₂—, —S(O)₀₋₂NR^{N}—, —NR^{N}S(O)₁₋₂NR^{N}—, —S(═O)(═NR^{N}), —NR^{N}S(O)(═NR^{N}), —S(═O)(═NR^{N})NR^{N}, NR^{N}S(O)(═NR^{N})R^{N}, —NR^{N}C(O)—, —NR^{N}C(O)NR^{N}—, or a bond;
   -L⁵ is a bond or C₁₋₄ alkylene;
   Rⁱ is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;

each occurrence of R^{N} is independently H or R^{d};

and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₄ alkyl), O, and S.

In one aspect, compounds of Formula (I), or a pharmaceutically acceptable salt thereof, are featured:

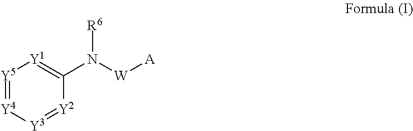

or a pharmaceutically acceptable salt thereof or a tautomer thereof,
wherein:

each of Y¹, Y², Y³, Y⁴, and Y⁵ is independently selected from the group consisting of N and CR¹;

W-A is defined according to (A) or (B) below:
(A)

W is selected from the group consisting of:
(a) *C(═O)NR^{N}, *C(═S)NR^{N}, *C(═NR^{N})NR^{N} (e.g., *C(═NCN)NH), *C(CNO₂)NR^{N}
(b) *S(O)₁₋₂NR^{N};

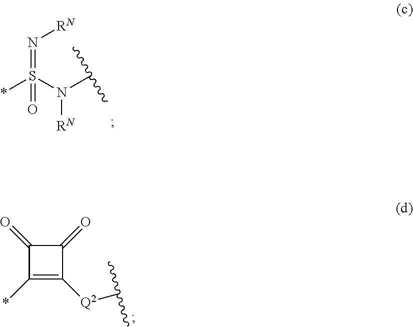

(e) *Q¹-Q²;
wherein the asterisk denotes point of attachment to NR⁶;

Q¹ is selected from the group consisting of:
(a) phenylene optionally substituted with from 1-2 independently selected R^{q1}; and
(b) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};
   Q² is selected from the group consisting of: a bond, NR^{N}, —S(O)₀₋₂—, —O—, and —C(═O)—;

### A is:

(i) —Y^{A1}-Y^{A1}, wherein:
Y^{A1} is a bond; or
Y^{A1} is C₁₋₆ alkylene, which is optionally substituted with from 1-6 substituents each independently selected from the group consisting of R^{a}; C₆₋aryl optionally substituted with 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; and
Y^{A2} is:
   (a) C₃₋₂₀ cycloalkyl, which is optionally substituted with from 1-4 R^{b},
   (b) C₆₋₂₀ aryl, which is optionally substituted with from 1-4 R^{c};
   (c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
   (d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

### OR

(ii) —Z¹-Z²-Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

### OR

(iii) C₁₋₁₀ alkyl, which is optionally substituted with from 1-6 independently selected R^{a},

### OR

(B)
   W is selected from the group consisting of:

(a) C₈₋₂₀ bicyclic or polycyclic aryl, which is optionally substituted with from 1-4 R^{c}; and

(b) bicyclic or polycyclic heteroaryl including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
NR^{e}R^{f},
—OH,
oxo,
S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″), and
—L³-L⁴-L⁵-Rⁱ;

or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;

each R² is independently selected from the group consisting of:
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″); and
—L³-L⁴-L⁵-Rⁱ;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{q1} is independently selected from the group consisting of:
(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;

each occurrence of R^{a} is independently selected from the group consisting of: —OH; —F; —Cl ; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;

each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl ; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); and (s) -L¹-L²-R^{h};

each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;

each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene optionally substituted with oxo;
-L² is —O—, —N(H)—, —S(O)₀₋₂—, or a bond;
R^{h} is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, and C₁₋₄ haloalkyl;
   -L¹ is a bond or C₁₋₃ alkylene optionally substituted with oxo;
   -L⁴ is —O—, —N(R^{N})—, —S(O)₀₋₂—, —NR^{N}S(O)₀₋₂—, —S(O)₀₋₂NR^{N}—, —NR^{N}S(O)₁₋₂NR^{N}—, —S(═O)(═NR^{N}), —NR^{N}S(O)(═NR^{N}), —S(═O)(═NR^{N})NR^{N}, NR^{N}S(O)(═NR^{N})NR^{N}, —NR^{N}C(O)—, —NR^{N}C(O)NR^{N}—, or a bond;
   -L⁵ is a bond or C₁₋₄ alkylene;
   Rⁱ is selected from:
C₃₋₈ cycloalkyl optionally substituted with from 1-4 substituents independently selected from the group consisting of halo, C₁₋₄ alkyl, and C₁₋₄ haloalkyl (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo, C₁₋₄ alkyl, and C₁₋₄ haloalkyl; and
C₆₋₁₀ aryl, which is optionally substituted with from 1-4 substituents independently selected from the group consisting of halo, C₁₋₄ alkyl, or C₁₋₄ haloalkyl;

each occurrence of R^{N} is independently H or R^{d};

and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₄ alkyl), O, and S.

The Variables Y¹-Y⁵ and R¹

In some embodiments, from 2-5 of Y¹, Y², Y³, Y⁴, and Y⁵ are independently CR¹.

In some embodiments, the ring including Y¹, Y², Y³, Y⁴, and Y⁵ is selected from the group consisting of:

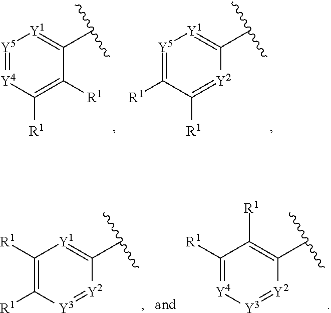

In some embodiments, each of Y¹, Y², Y³, Y⁴, and Y⁵ is an independently selected CR¹ (i.e., the ring including Y¹, Y², Y³, Y⁴, and Y⁵ is

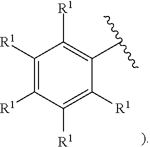

In certain of these embodiments, the ring including Y¹, Y², Y³, Y⁴, and Y⁵ is selected from the group consisting of:

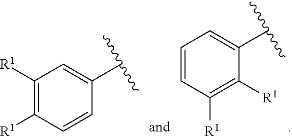

In certain embodiments (when the ring including Y¹, Y², Y³, Y⁴, and Y⁵ is

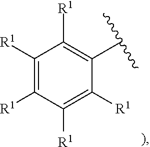

the ring including Y¹, Y², Y³, Y⁴, and Y⁵ is selected from the group consisting of:

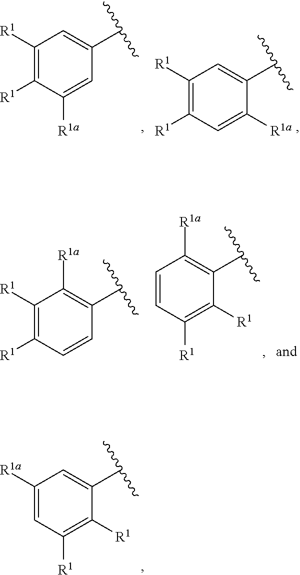

wherein each R^{1a} is an independently selected R¹.

In certain embodiments (when the ring including Y¹, Y², Y³, Y⁴, and Y⁵ is

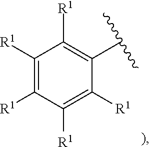

the ring including Y¹, Y², Y³, Y⁴, and Y⁵ is selected from the group consisting of:

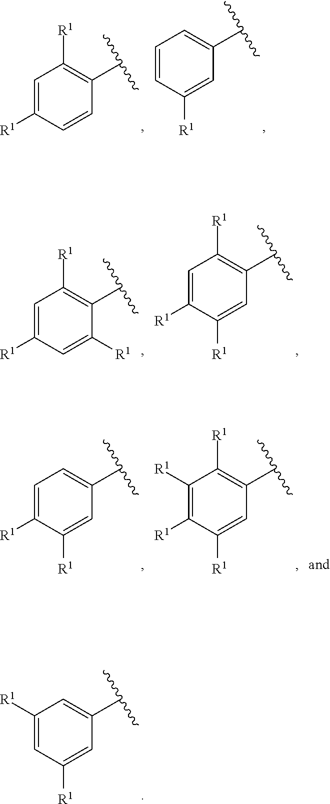

In some embodiments, from 1-2 (e.g., 1 or 2) of Y¹, Y², Y³, Y⁴, and Y⁵ is independently N; and each of the remaining Y¹, Y², Y³, Y⁴, and Y⁵ is an independently selected CR¹.

In certain of these embodiments, the ring including Y¹, Y², Y³, Y⁴, and Y⁵ is pyridinyl.

As a non-limiting example of the foregoing embodiments, the ring including Y¹, Y², Y³, Y⁴, and Y⁵ is pyridin-2-yl (i.e.,

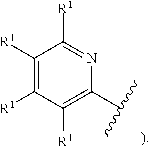

In certain embodiments (when the ring including Y¹, Y², Y³, Y⁴, and Y⁵ is pyridin-2-yl), the ring including Y¹, Y², Y³, Y⁴, and Y⁵ is selected from the group consisting of:

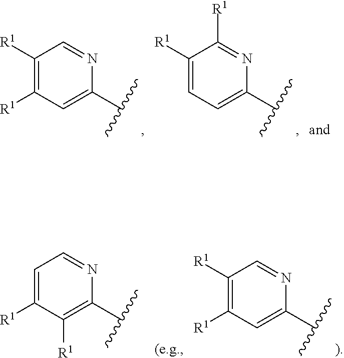

In certain embodiments, the ring including Y¹, Y², Y³, Y⁴, and Y⁵ is pyridin-3-yl (i.e.,

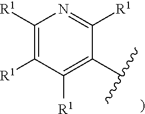

or pyridin-4-yl (i.e.,

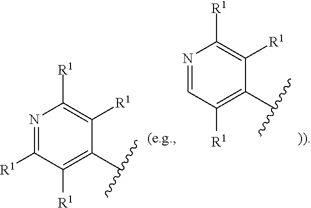

In certain of these embodiments, the ring including Y¹, Y², Y³, Y⁴, and Y⁵ is selected from the group consisting of:

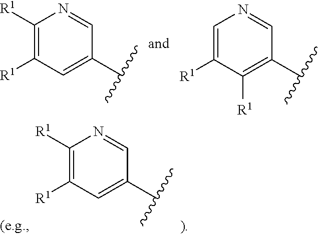

In certain embodiments (when the ring including Y¹, Y², Y³, Y⁴, and Y⁵ is pyridin-3-yl or pyridin-4-yl), ring including Y¹, Y², Y³, Y⁴, and Y⁵ is selected from the group consisting of:

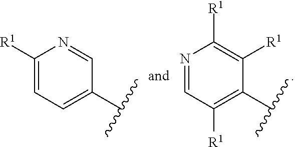

In certain embodiments, the ring including Y¹, Y², Y³, Y⁴, and Y⁵ is pyrimidinyl (e.g.,

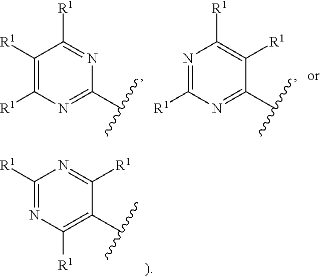

As a non-limiting example of the foregoing embodiments, the ring including Y¹, Y², Y³, Y⁴, and Y⁵ is

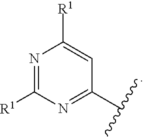

In some embodiments, a pair of R¹ on adjacent atoms, taken together with the atoms connecting them, form a ring including from 4-15 (e.g., 5-12 (e.g., 5, 6, 7, 8, 9, or 10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a ring including from 4-12 (e.g., 4, 5, 6, 7, 8, 9, or 10) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a ring including from 5-12 (e.g., 5, 6, 7, 8, 9, or 10) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

In certain embodiments, a pair of R¹ on adjacent atoms, taken together with the atoms connecting them, form a ring including from 5-6 ring atoms (e.g., an aromatic ring including from 5-6 ring atoms), wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a ring including 5 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of the foregoing embodiments, wherein a pair of R¹ on adjacent atoms, taken together with the atoms connecting them, form an aromatic ring including 5 ring atoms, wherein from 1-2 (e.g., 1; or e.g., 2) ring atoms are heteroatoms each independently selected from the group consisting of N, N(H), N(R^{d}), O, and S(O)₀₋₂; and wherein the ring is optionally substituted with from 1-4 independently selected R².

In certain embodiments, a pair of R¹ on adjacent atoms, taken together with the atoms connecting them, form a pyrrolyl ring optionally substituted with from 1-2 independently selected R².

In certain of these embodiments, a pair of R¹ on adjacent atoms, taken together with the atoms connecting them, form:

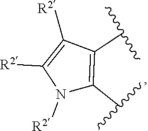

wherein each R^{2′} is independently H or R² (e.g.,

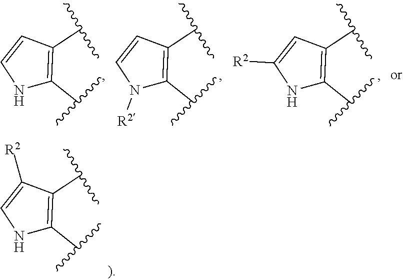

In certain embodiments, a pair of R¹ on adjacent atoms, taken together with the atoms connecting them, form a pyrazolyl, imidazolyl, or thiazolyl ring optionally substituted with from 1-2 independently selected R².

In certain of these embodiments, a pair of R¹ on adjacent atoms, taken together with the atoms connecting them, form:

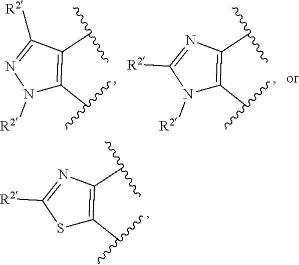

wherein each R^{2′} is independently H or R² (e.g.,

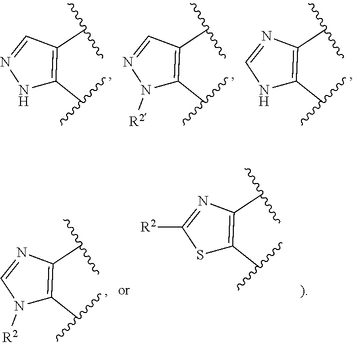

In certain embodiments, a pair of R¹ on adjacent atoms, taken together with the atoms connecting them, form a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R².

As a non-limiting example of the foregoing embodiments, a pair of R¹ on adjacent atoms, taken together with the atoms connecting them, form:

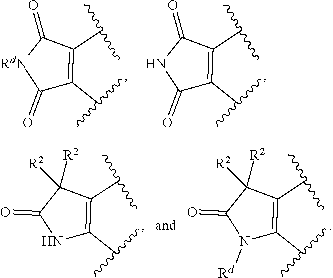

In certain embodiments, a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; wherein one ring atom is —O— or S(O)₀₋₂; and wherein the ring is optionally substituted with from 1-2 independently selected R² (e.g., tetrahydrofuranyl (e.g.,

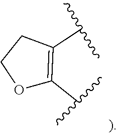 [SMILES: *C1=C(*)OCC1]

In certain embodiments, a pair of R¹ on adjacent atoms, taken together with the atoms connecting them, form a ring including 6 ring atoms (e.g., an aromatic ring including 6 ring atoms (e.g., pyridinyl or pyrimidinyl), wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form pyridinyl (including pyridonyl), which is optionally substituted with from 1-3 independently selected R².

As non-limiting examples of the foregoing embodiments, a pair of R¹ on adjacent atoms, taken together with the atoms connecting them, form:

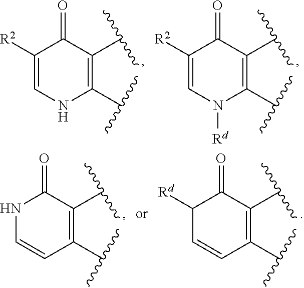

In certain embodiments, a pair of R¹ on adjacent atoms, taken together with the atoms connecting them, form a cycloalkyl ring including from 5-6 ring atoms; and wherein the ring is optionally substituted with from 1-4 independently selected R².

In certain of these embodiments a air of R¹ on adjacent atoms, taken together with the atoms connecting them, form

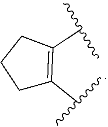 [SMILES: *C1=C(*)CCC1]

In certain embodiments, a pair of R¹ on adjacent atoms, taken together with the atoms connecting them, form a ring including from 7-12 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, a pair of R¹ on adjacent atoms, taken together with the atoms connecting them, form a ring including from 8-12 (e.g., 8; or e.g., 9-12 (e.g., 9, 10, 11, or 12)) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of the foregoing embodiments, a pair of R¹ on adjacent atoms, taken together with the atoms connecting them, form a spirocyclic bicyclic ring including from 8-12 (e.g., 9-12 (e.g., 9, 10, 11, or 12)) ring atoms, wherein from 0-2 ring atoms are heteroatoms each independently selected from the group consisting of N, N(H), N(R^{d}), 0, and S(O)₀₋₂; and wherein the ring is optionally substituted with from 1-4 independently selected R².

As a non-limiting example of the foregoing embodiments, a pair of R¹ on adjacent atoms, taken together with the atoms connecting them, form:

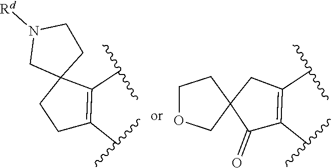

each of which is further optionally substituted with from 1-2 independently selected R².

In some embodiments, the compound has the following formula:

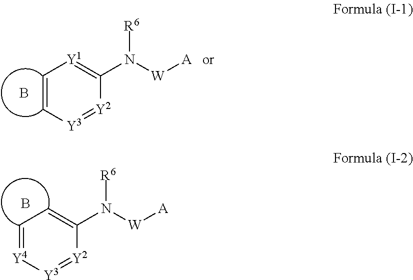

wherein ring B is a ring (e.g., monocyclic ring, bicyclic ring, or tricyclic ring) including from 4-15 (e.g., 5-12 (e.g., 5-10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, the compound has the following formula:

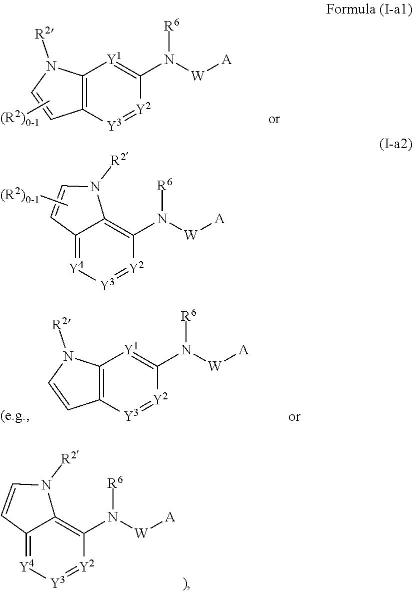

wherein R^{2′} is H or R² (e.g., R^{2′} is H) (in certain embodiments, the compound has Formula (I-a1); in certain of these embodiments, R^{2′} is H). In certain embodiments of Formula (I-a1), Y³ is CR¹, wherein the R¹ is other than H, OH, or oxo. For example, Y³ is C-halo or C-cyano.

In certain embodiments (e.g., when the compound has Formula (I-1) or (I-2)), the compound has the following formula:

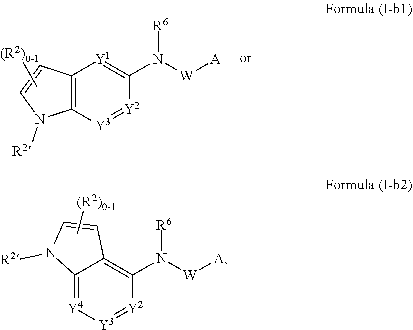

wherein R^{2′} is H or R² (e.g.,

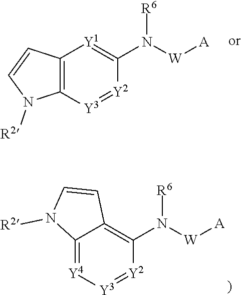

(e.g., R^{2′} is H) (in certain embodiments, the compound has Formula (I-b1); in certain of these embodiments, R^{2′} is H).

In certain embodiments (e.g., when the compound has Formula (I-1) or (I-2)), the compound has the following formula:

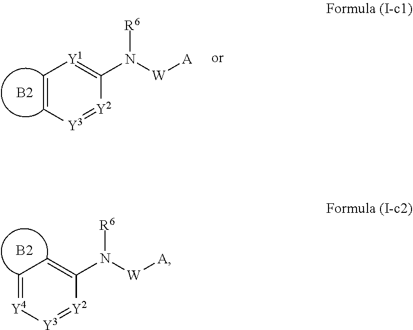

wherein B2 is an aromatic ring including 5 ring atoms, wherein from 1-2 (e.g., 2) ring atoms are heteroatoms each independently selected from the group consisting of N, N(H), N(R^{d}), O, and S(O)₀₋₂, provided that B2 is other than pyrrolyl; and wherein the ring is optionally substituted with from 1-4 independently selected R².

In certain of these embodiments, B2 is pyrazolyl, imidazolyl, or thiazolyl ring optionally substituted with from 1-2 independently selected R².

By way of non-limiting examples, B2 is

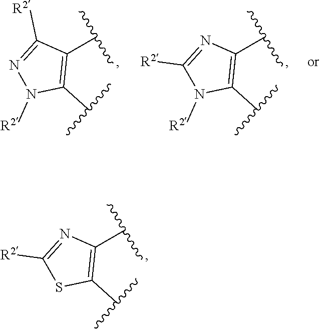

wherein each R^{2′} is independently H or R² (e.g.,

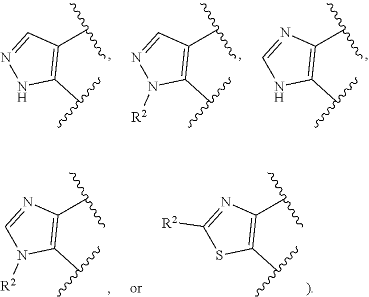

In certain embodiments (e.g., when the compound has Formula (I-1) or (I-2)), the compound has the following formula:

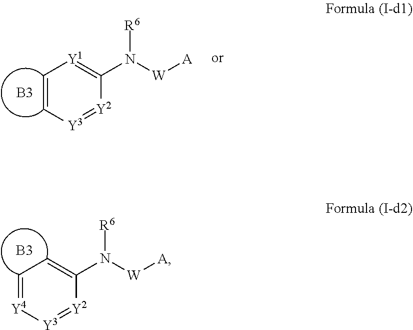

wherein B3 is selected from the group consisting of:
a) a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².
b) a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, B3 is a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain embodiments, B3 is a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain embodiments, B3 is a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R² (e.g.,

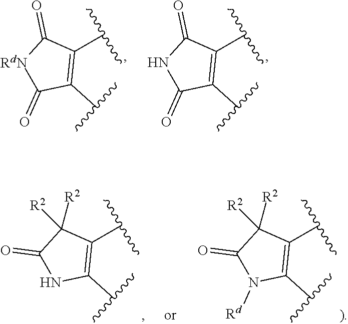

In certain embodiments, B3 is non-aromatic ring including 5 ring atoms, wherein from 0-1 ring atoms is a heteroatom selected from the group consisting of N, N(H), N(R^{d}), O, and S(O)₀₋₂; wherein the ring is optionally substituted with from 1-2 independently selected R² (e.g.,

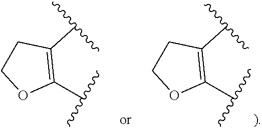 [SMILES: *C1=C(*)OCC1 ; *C1=C(*)OCC1]

In certain embodiments, B3 is a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, B3 is a spirocyclic bicyclic ring including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

As non-limiting examples of the foregoing embodiments, B3 is

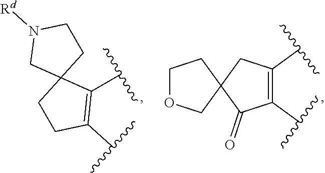

each of which is further optionally substituted with from 1-2 independently selected R².

In certain embodiments, the compound has the following formula:

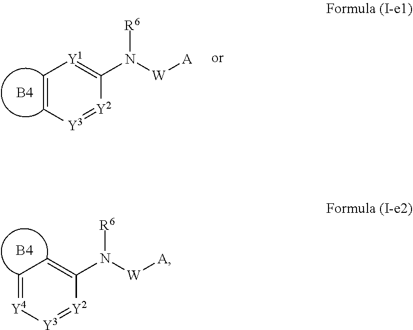

wherein B4 is an aromatic ring including 6 ring atoms, wherein from 0-2 ring atoms are heteroatoms each independently selected from the group consisting of N, N(H), and N(R^{d}); and wherein the ring is optionally substituted with from 1-4 independently selected R².

In certain of these embodiments, B4 is pyridinyl (including pyridonyl), which is optionally substituted with from 1-3 independently selected R² (e.g.,

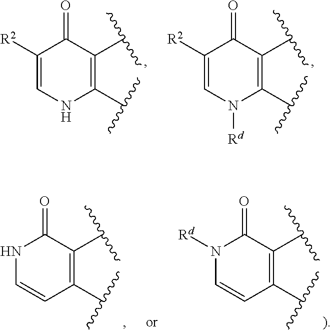

In certain embodiments, when the compound is of formula (I-1), (I-a1), (I-b1), (I-c1), (I-d1), or (I-e1), each of Y¹, Y², and Y³ is an independently selected CR¹; and when the compound is of formula (I-2), (I-a2), (I-b2), (I-c2), (I-d2), or (I-e2), each of Y², Y³, and Y⁴ is an independently selected CR¹.

In certain embodiments, when the compound is of formula (I-1), (I-a1), (I-b1), (I-c1), (I-d1), or (I-e1), one of Y¹, Y², and Y³ is N; and each of the remaining of Y¹, Y², and Y³ is an independently selected CR¹; and

when the compound is of formula (I-2), (I-a2), (I-b2), (I-c2), (I-d2), or (I-e2), one of Y², Y³, and Y⁴ is N; and each of the remaining of Y², Y³, and Y⁴ is an independently selected CR¹.

In some embodiments, the compound has Formula (I-a1-b):

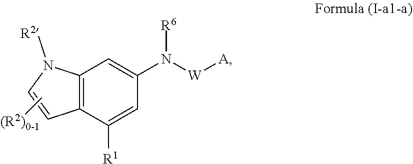

wherein R^{2′} is H or R².

In certain of these embodiments, the compound has Formula (I-a1-b):

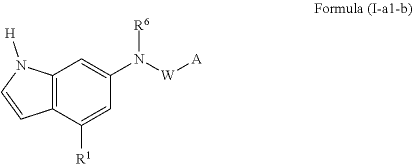

(e.g., R¹ is other than hydrogen (e.g., R¹ is other than hydrogen; and A is Y^{A1}-Y^{A2} (e.g., Y² is optionally substituted aryl or optionally substituted heteroaryl such as optionally substituted pyridyl as defined herein)).

In certain embodiments, the compound has Formula (I-a1-c):

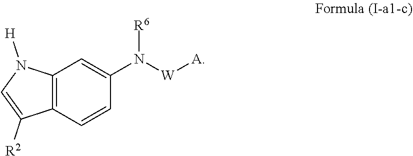

In certain embodiments, the compound has Formula (I-a1-d):

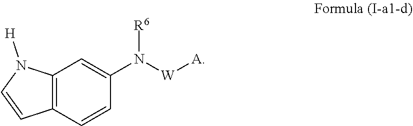

In certain embodiments, the compound has Formula (I-a1-e):

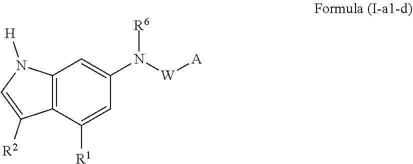

(e.g., R¹ is other than hydrogen (e.g., R¹ is other than hydrogen; and A is Y^{A1}-Y^{A2} (e.g., Y^{A2} is optionally substituted aryl or optionally substituted heteroaryl such as optionally substituted pyridyl as defined herein)).

In some embodiments, the compound has Formula (I-b1-a):

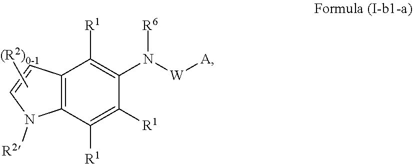

wherein R^{2′} is H or R².

In certain embodiments, the compound has Formula (I-b1-b):

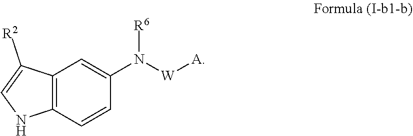

In certain embodiments, the compound has Formula (I-b1-c):

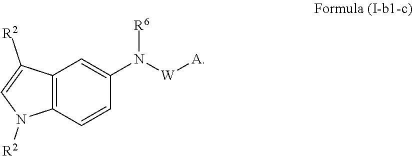

In certain embodiments, the compound has Formula (I-b1-d):

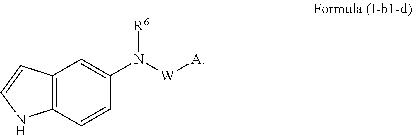

In some embodiments, each occurrence of R¹ that is not taken together with the atom to which it is attached in ring formation is independently selected from the group consisting of:
H;
halo;
cyano;
C₁₋₆ alkyl optionally substituted with 1-2 R^{a};
C₂₋₆ alkenyl;
C₂₋₆ alkynyl;
C₁₋₄haloalkyl;
C₁₋₄ alkoxy;
C₁₋₄haloalkoxy;
—S(O)₁₋₂(C₁₋₄ alkyl),
—NR^{e}R^{f},
—OH,
oxo,
—S(O)₁₋₂(NR′R″),
—C(═O)(C₁₋₄ alkyl),
—C(═O)O(C₁₋₄ alkyl),
—C(═O)OH,
—C(═O)N(R′)(R″), and
—L³-L⁴-Rⁱ.

In some embodiments, each occurrence of R¹ that is not taken together with the atom to which it is attached in ring formation is H.

In some other embodiments, from 1-3 (e.g., 1, 2, or 3) occurrences of R¹ that is not taken together with the atom to which it is attached in ring formation is other than H; and each of the remaining occurrence of R¹ that is not taken together with the atom to which it is attached in ring formation is H.

In certain of these embodiments, each R¹ that is not taken together with the atom to which it is attached in ring formation is other than H; and each of the remaining occurrence of R¹ that is not taken together with the atom to which it is attached in ring formation is H.

In some embodiments, one occurrence of R¹ is halo (e.g., F or Cl ).

In some embodiments, one occurrence of R¹ is NR^{e}R^{f} (e.g., NHAc) or C₁₋₄ alkoxy (e.g., methoxy).

In some embodiments, one occurrence of R¹ is C₁₋₆ alkyl optionally substituted with 1-2 R^{a} (e.g., methyl, CH₂OH, or CH₂CH₂OH).

In some embodiments, one occurrence of R¹ is cyano.

In some embodiments, one occurrence of R¹ is -L³-L⁴-Rⁱ (e.g., -L³ is a bond; and -L¹ is —O— (e.g., R¹ is phenoxy)).

In certain of these embodiments, -L³ is a bond; and -L⁴ is —O— (e.g., R¹ is phenoxy).

In certain other embodiments, -L³ is a bond; and -L⁴ is a bond (e.g., R¹ is pyrazolyl or phenyl).

In some embodiments, one occurrence of R¹ is selected from the group consisting of C(═O)OH and C(═O)O(C₁₋₄ alkyl).

### The Variable R²

In some embodiments, each occurrence of R² is independently selected from the group consisting of:
halo;
cyano;
C₁₋₆ alkyl optionally substituted with 1-2 R^{a};
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″); and
—L³-L⁴-L⁵-R.

In some embodiments, one occurrence of R² is halo (e.g., F, Cl , or Br (e.g., F or Cl ) or cyano.

In some embodiments, one occurrence of R² is C₁₋₆ alkyl optionally substituted with 1-2 R^{a}. In certain of these embodiments, each occurrence of R^{a} is independently —F, —Cl, —OH, C₁₋₄ alkoxy, C₁₋₄ haloalkoxy, and —NR^{e}R^{f} (e.g., R² is methyl, CH₂OH, or CH₂CH₂OH).

In some embodiments, one occurrence of R² is oxo; or wherein one occurrence of R² is OH.

In some embodiments, one occurrence of R² is NR^{e}R^{f}.

In certain of these embodiments, each of R^{e} and R^{f} is independently selected from H; C₁₋₆ alkyl optionally substituted with from 1-2 substituents each independently selected from halo, OH, C₁₋₄ alkoxy, C₁₋₄ haloalkoxy, and CN; —C(O)(C₁₋₄ alkyl); —C(O)O(C₁₋₄ alkyl); —CON(R′)(R″); —S(O)₁₋₂(C₁₋₄ alkyl); and —S(O)(═NR′)(C₁₋₄ alkyl).

In certain of the foregoing embodiments, R^{e} and R^{f} is H (e.g., NR^{e}R^{f} is NHAc, NHS(O)₂Me, NHS(O)(═NH)Me, or NH(CH₂CH₂OH)).

In some embodiments, one occurrence of R² is -L³-L⁴-L⁵-Rⁱ.

In certain of these embodiments, -L³ of R² is a bond. In certain other embodiments, -L³ of R² is C₁₋₃ alkylene (e.g., CH₂).

In certain embodiments, -L¹ of R² is NR^{N} (e.g., NH).

In certain embodiments, -L¹ of R² is a bond.

In certain embodiments, -L¹ of R² is selected from the group consisting of a —NR^{N}C(O), —NR^{N}S(O)₀₋₂— or —NR^{N}S(═O)(═NR^{N}) (e.g., R^{N} is H).

In certain embodiments, -L¹ of R² is selected from the group consisting of NR^{N}S(═O)═NR^{N})NR^{N}, —NR^{N}S(O)₁₋₂NR^{N}—, and —NR^{N}C(O)NR^{N}— (e.g., R^{N} is H). In certain embodiments, -L⁵ is a bond.

In certain other embodiments, -L⁵ is C₁₋₃ alkylene (e.g., —CH(CH₃)CH₂—). In certain embodiments, R¹ of R² is C₃₋₈(e.g., C₆) cycloalkyl optionally substituted with from 1-4 (e.g., from 1-2) substituents independently selected from the group consisting of halo, C₁₋₄ alkyl, and C₁₋₄ haloalkyl (in certain embodiments, it is provided that when R^{h} is C₃₋₆ cycloalkyl optionally substituted with from 1-4 substituents independently selected C₁₋₄ alkyl, -L¹ is a bond, or -L² is —O—, —N(H)—, or —S—).

In certain embodiments, R¹ of R² is C₆₋₁₀ (e.g., C₆) aryl, which is optionally substituted with from 1-4 (e.g., from 1-2) substituents independently selected from the group consisting of halo, C₁₋₄ alkyl, and C₁₋₄ haloalkyl.

In certain embodiments, R¹ of R² is heteroaryl including from 5-6 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl; and C₁₋₄ haloalkyl.

By way of non-limiting examples, when R² is -L³-L⁴-L⁵-Rⁱ, R² can be:

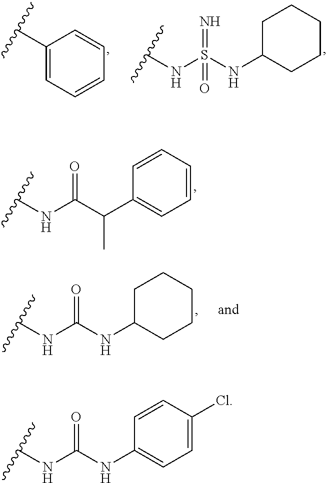

As further non-limiting examples, when R² is -L³-L⁴-L⁵-R, R² can be selected from the group consisting of:

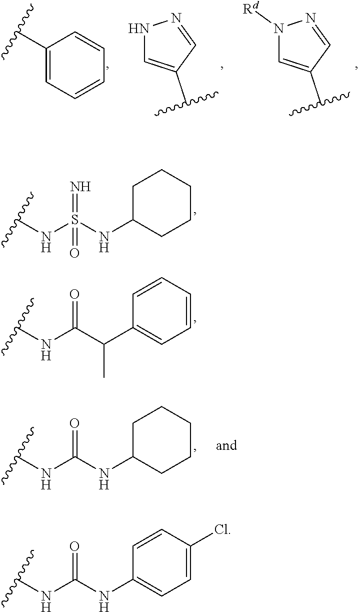

In some embodiments, one occurrence of R² is C(O)OH.

Embodiments when W-A is Defined According to (A)

In some embodiments, W-A as defined according to (A).

The Variable W

In some embodiments, W is selected from the group consisting of *C(═O)NR^{N}, *C(═S)NR^{N}, *C(═NR^{d})NR^{N}, *C(═CNO₂)NR^{N}.

In certain embodiments, W is *C(═O)NR^{N}.

In certain of these embodiments, W is *C(═O)NH or *C(═O)N(C₁₋₃ alkyl).

As a non-limiting example of the foregoing embodiments, W is *C(═O)NH.

In certain embodiments, W is *S(O)₁₋₂NR^{N}. In certain of these embodiments, W is *S(O)₂NR^{N} (e.g., *S(O)₂NH).

In certain embodiments, W is

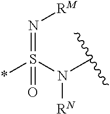

(e.g., each R^{N} is H).

In certain embodiments, W is

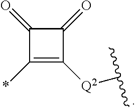

In certain of these embodiments, Q² is NR^{N}. In certain embodiments, Q² is NH or N(C₁₋₃ alkyl). For example, Q² is NH.

In certain embodiments, W is -Q¹-Q². In certain of these embodiments, -Q¹ is heteroarylene including from 5-6 ring atoms, wherein from 1-4 ring atoms are heteroatoms, each independently selected from the group consisting of N, N(H), N(R^{d}), O, and S, and wherein the heteroarylene ring is optionally substituted with from 1-4 independently selected R^{q1}.

In certain of the foregoing embodiments, Q¹ is heteroarylene including 6 ring atoms, wherein from 1-3 (e.g., 1-2) ring atoms are ring nitrogen atoms, and wherein the heteroarylene ring is optionally substituted with from 1-2 independently selected R^{q1}.

In certain embodiments, Q¹ is heteroarylene including 6 ring atoms, wherein from 1-3 (e.g., 1-2) ring atoms are ring nitrogen atoms, and wherein the heteroarylene ring is optionally substituted with from 1-2 independently selected R^{q1}.

In certain embodiments, Q¹ is pyridylene or pyrimidinylene, each of which is optionally substituted with 1-2 independently selected R^{q1}.

As non-limiting examples of the foregoing embodiments, Q¹ is selected from the group consisting of:

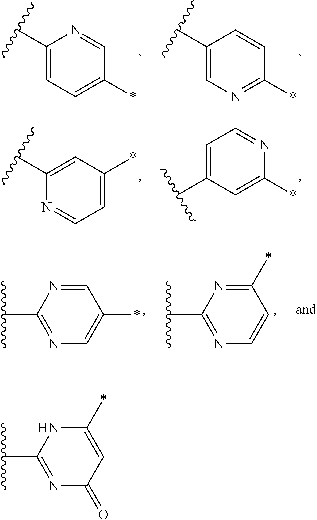

each of which is optionally substituted with 1-2 independently selected R^{q1}, wherein the asterisk denotes point of attachment of Q² (e.g.,

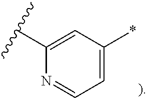 [SMILES: *c1ccnc(*)c1]

In certain embodiments (when W is -Q¹-Q²), each R^{q1} is independently selected from the group consisting of: halo; cyano; C₁₋₁₀ alkyl which is optionally substituted with from 1-6 independently selected R^{a} (e.g., unsubstituted C₁₋₁₀ alkyl); C₃₋₆ cycloalkyl; and oxo.

In certain embodiments (when W is -Q¹-Q²), Q² is a bond.

In certain embodiments (when W is -Q¹-Q²), Q² is —O—, —NH—, or —S(O)₀₋₂(e.g., Q² is —O—; or Q² is —NH—; or Q² is —S(O)₂—).

The Variable A

In some embodiments, A is —Y^{A1}-Y^{A2}.

In certain of these embodiments, Y^{A1} is a bond.

In certain other embodiments, Y^{A1} is C₁₋₆ alkylene, which is optionally substituted with from 1-4 R^{a}.

In certain of the foregoing embodiments, Y^{A1} is C₁₋₆ alkylene.

In certain embodiments, Y^{A1} is C₁₋₆ alkylene which is optionally substituted with from 1-2 R^{a}.

As a non-limiting example, Y^{A1} can be —CH₂—, —CH₂CH₂—, —CH₂CH₂CH₂—, —CH(CF₃)—, —CH₂CH(OH)—,

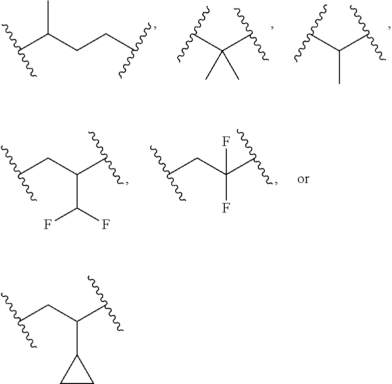

(e.g., Y^{A1} is CH₂).

For example, Y^{A1} can be —CH₂— or —CH₂CH₂—.

In certain other embodiments, Y^{A1} is Y^{A3}-Y^{A4}—Y^{A5}.

In certain of these embodiments, Y^{A3} is C₂₋₃ alkylene; and/or Y^{A4} is —O— or —S—; and/or Y^{A5} is a bond.

As a non-limiting example of the foregoing embodiments, Y^{A1} can be

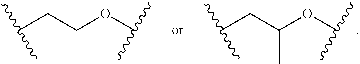

In certain of these embodiments, Y^{A3} is C₂₋₃ alkylene; and/or Y^{A4} is —O— or —S—; and/or Y^{A5} is C₁₋₂ alkylene.

As a non-limiting example, Y^{A1} can be

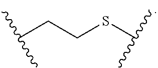 [SMILES: *CCS*]

As non-limiting examples when Y^{A1} is C₁₋₆ alkylene, which is optionally substituted with from 1-4 R^{a}, Y^{A1} is —CH₂—, —CH₂CH₂—, —CH₂CH₂CH₂—, —CH(CF₃)—, —CH₂CH(OH)—, or

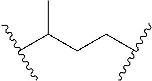 [SMILES: *CCC(*)C]

### (e.g., CH₂).

In some embodiments, Y^{A2} is C₆₋₁₀ aryl, which is optionally substituted with from 1-3 R^{c}.

In certain embodiments, Y^{A2} is C₆ aryl.

In certain embodiments, Y^{A2} is C₆ aryl, which is substituted with from 1-3 R^{c}.

In certain embodiments, Y^{A2} is phenyl substituted with from 1-3 (e.g., 1 or 2) R^{c}, wherein one R^{c} is at the ring carbon para to the point of attachment to Y^{A1}

In certain embodiments, Y^{A2} is phenyl substituted with from 1-3 (e.g., 1 or 2) R^{c}, wherein from 1-2 (e.g., 1) R^{c} is at the ring carbons meta to the point of attachment to Y^{A1}

In certain embodiments, Y^{A2} is phenyl substituted with from 1-3 (e.g., 1 or 2) R^{c}, wherein from 1-2 (e.g., 1) R^{c} is at the ring carbons ortho to the point of attachment to Y^{A1}.

In certain embodiments, Y^{A2} is C₇₋₁₀ bicyclic aryl, which is optionally substituted with from 1-3 R^{c} (e.g., Y^{A2} is naphthyl (e.g.,

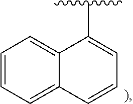 [SMILES: *c1cccc2ccccc12]

indacenyl (e.g.,

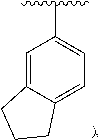 [SMILES: *c1ccc2c(c1)CCC2]

or tetrahydronapthyl, each of which is optionally substituted with from 1-3 R^{c}).

In some embodiments, Y^{A2} is heteroaryl including from 5-14 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}.

In certain embodiments, Y^{A2} is heteroaryl including 5 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-3 independently selected R^{c}.

In certain of these embodiments, Y^{A2} is thiazolyl, thiadiazolyl, isoxazolyl triazolyl, or pyrazolyl, each of which is optionally substituted with from 1-2 (e.g., 1) independently selected R^{c} (e.g., Y^{A2} is pyrazolyl which is optionally substituted with from 1-2 (e.g., 1) independently selected R^{c} (e.g., Y^{A2} is

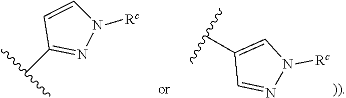

In certain of these embodiments, Y^{A2} is thiazolyl, triazolyl, or pyrazolyl, each of which is optionally substituted with from 1-2 (e.g., 1) independently selected R^{c} (e.g., Y^{A2} is pyrazolyl which is optionally substituted with from 1-2 (e.g., 1) independently selected R^{c} (e.g., Y^{A2} is

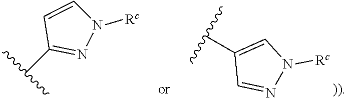

In certain embodiments, Y^{A2} is heteroaryl including 6 ring atoms (e.g., pyridyl or pyrimidinyl (e.g., pyridyl (e.g.,

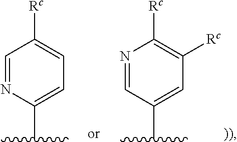

wherein from 1-2 ring nitrogen atoms, and wherein the heteroaryl ring is optionally substituted with from 1-3 independently selected R^{c}.

In certain of these embodiments, Y^{A2} is substituted with from 1-3 independently selected R^{c}; and one occurrence of R^{c} is at the ring carbon atom para to the point of attachment to Y^{A1}.

In certain embodiments (when Y^{A2} is heteroaryl including 6 ring atoms (e.g., pyridyl or pyrimidinyl (e.g., pyridyl (e.g.,

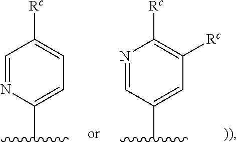

wherein from 1-2 ring nitrogen atoms, and wherein the heteroaryl ring is optionally substituted with from 1-3 independently selected R^{c}), Y^{A2} is substituted with from 1-3 independently selected R^{c}; and from 1-2 occurrences of R^{c} is at the ring carbon atom meta to the point of attachment to Y^{A1}

In certain embodiments, Y^{A2} is bicyclic or tricyclic heteroaryl including from 7-14 (e.g., 9-12 (e.g., 9, 10, 11, or 12)) ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c} (e.g., Y^{A2} is

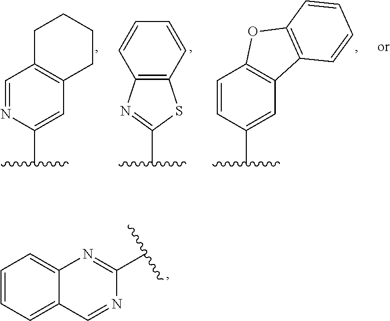

each of which is optionally substituted with from 1-2 independently selected R^{c}).

Embodiments when W-A is Defined According to (B)

In some embodiments, W-A as defined according to (B).

In certain of these embodiments, W is C₈₋₁₀ bicyclic arylene, which is optionally substituted with from 1-4 R^{c}.

In certain embodiments when W-A is defined according to (B), W is bicyclic heteroarylene including from 8-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}.

In certain of these embodiments, W is heteroarylene including from 9-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-2 independently selected R^{c}.

In certain of the foregoing embodiments, W is selected from the group consisting of quinolinylene, isoquinolinylene, and quinazolinylene, each of which is optionally substituted with from 1-2 independently selected R^{c}.

As a non-limiting example of the foregoing embodiments, W can be

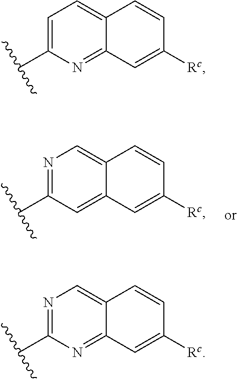

In certain embodiments when W-A is as defined according to (B), A is H.

In certain other embodiments when W-A is as defined according to (B), A is as defined for (A). For example, A can be C₁₋₂₀ alkyl (e.g., C₁₋₃ alkyl), which is optionally substituted with from 1-6 independently selected R^{a},

The Variable R^{c}

In some embodiments, each occurrence of R^{c} is independently selected from the group consisting of:

halo;

cyano;

C₁₋₁₀ alkyl which is optionally substituted with from 1-6 independently selected R^{a};

C₂₋₆ alkenyl;

C₂₋₆ alkynyl;

C₁₋₄ alkoxy;

C₁₋₄ haloalkoxy;

—S(O)₁₋₂(C₁₋₄ alkyl);

—S(O)₁₋₂(C₁₋₄ haloalkyl);

—NR^{e}R^{f};

—C₁₋₄ thioalkoxy;

—C₁₋₄ thiohaloalkoxy;

—SF₅;

—C(═O)(C₁₋₁₀ alkyl);

—C(═O)(OH);

—C(═O)O(C₁₋₄ alkyl); and

-L¹-L²-R^{h}.

In certain embodiments, each occurrence of R^{c} is independently selected from the group consisting of:

halo;

cyano;

C₁₋₁₀ alkyl which is optionally substituted with from 1-6 independently selected R^{a};

C₂₋₆ alkenyl;

C₂₋₆ alkynyl;

C₁₋₄ alkoxy;

C₁₋₄ haloalkoxy;

—S(O)₁₋₂(C₁₋₄ alkyl);

—NR^{e}R^{f};

—C₁₋₄ thioalkoxy;

—C(═O)(C₁₋₁₀ alkyl);

—C(═O)(OH);

—C(═O)(C₁₋₄ alkyl); and

-L¹-L²-R^{h}.

In certain embodiments, one occurrence of R^{c} is halo.

In certain embodiments, one occurrence of R^{c} is cyano.

In certain embodiments, one occurrence of R^{c} is C₁₋₁₀ alkyl which is optionally substituted with from 1-6 independently selected R^{a}.

In certain of these embodiments, one occurrence of R^{c} is unsubstituted C₁₋₁₀ alkyl (e.g., C₂, C₃, C₄, C₅, C₆, or C₇₋₁₀).

As non-limiting examples of the foregoing embodiments, one occurrence of R^{c} is ethyl, propyl (e.g., n-propyl), butyl (e.g., n-butyl, iso-butyl, sec-butyl, tert-butyl), pentyl, or octyl (e.g., n-octyl) (e.g., R^{c} is butyl (e.g., n-butyl)).

In certain embodiments, one occurrence of R^{c} is unsubstituted C₆₋₁₀ alkyl (e.g., straight-chain C₆₋₁₀ alkyl).

In certain embodiments, one occurrence of R^{c} is C₁₋₁₀ alkyl which is substituted with from 1-6 independently selected R^{a}.

In certain of these embodiments, each occurrence of R^{a} is independently selected from —F, —Br, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl. As a non-limiting example, each R^{a} is —F.

As non-limiting examples of the foregoing embodiments, one occurrence of R^{c} is selected from: CF₃, CHF₂, CH₂CF₃, CH₂CH₂CF₃, CH₂CH₂CH₂OH, CH₂CH₂OH, CH₂OH, CH₂CH₂OMe, CH₂OEt, CH₂OCH₂CH₂CH₃, CH(OH)CH₂CH₃, CH₂NMe₂, CH₂CH₂NMe₂, and

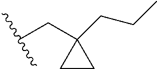 [SMILES: *CC1(CCC)CC1]

(e.g., R^{c} is CF₃).

In certain embodiments, one occurrence of R^{c} is —SF₅.

In certain embodiments, one occurrence of R^{c} is —S(O)₁₋₂(NR′R″)(e.g.,

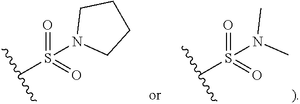

In certain embodiments, one occurrence of R^{c} is S(O)₁₋₂(C₁₋₄ alkyl) or S(O)₁₋₂(C₁₋₄ haloalkyl) (e.g., S(O)₂CF₃). In certain embodiments, one occurrence of R^{c} is C₁₋₄ alkoxy or C₁₋₄ haloalkoxy (e.g., C₁₋₄ haloalkoxy such as OCF₃, OCF₂H, OCH₂CF₃, and OCH₂CF₂H). In certain embodiments, one occurrence of R^{c} is C₂₋₆ alkenyl or C₂₋₆ alkynyl (e.g., C₂₋₆ alkynyl (e.g., acetylenyl)). In certain embodiments, one occurrence of R^{c} is —C(═O)(C₁₋₁₀ alkyl) (e.g., —C(═O)(C₃₋₁₀ alkyl) (e.g., —C(═O)CH₂CH₂CH₂CH₂CH₂CH₂CH₂)).

In certain embodiments, one occurrence of R^{c} is -L¹-L²-R^{h}. In certain of these embodiments, L¹ is a bond. In certain other embodiments, L¹ is CH₂, CH₂CH₂, or C(═O). In certain embodiments (when one occurrence of R^{c} is -L¹-L²-R^{h}), L² is a bond. In certain other embodiments, L² is —O—. In certain embodiments (when one occurrence of R^{c} is -L¹-L²-R^{h}), L¹ is a bond; and L² is a bond. In certain other embodiments, L¹ is a bond; and L² is —O—.

In certain embodiments (when one occurrence of R^{c} is -L¹-L²-R^{h}), R^{h} is C₃₋₈ cycloalkyl optionally substituted with from 1-4 substituents independently selected from the group consisting of halo, C₁₋₄ alkyl, and C₁₋₄ haloalkyl.

In certain of these embodiments (e.g., when -L¹ is a bond; and -L² is a bond), R^{h} is C₃₋₆ cycloalkyl optionally substituted with from 1-4 substituents independently selected from the group consisting of halo, C₁₋₄ alkyl, and C₁₋₄ haloalkyl (e.g.,

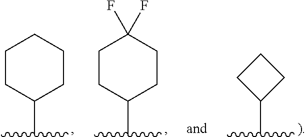

For example, R^{h} is selected from the group consisting of:

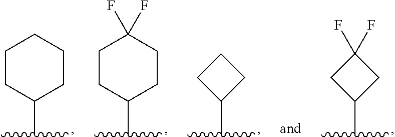

In certain embodiments, R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

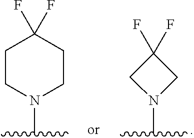 [SMILES: *N1CCC(F)(F)CC1 ; *N1CC(F)(F)C1]

In certain embodiments (when one occurrence of R^{c} is -L¹-L²-R^{h}), R^{h} is C₆₋₁₀ aryl, which is optionally substituted with from 1-4 substituents independently selected from the group consisting of halo, cyano, C₁₋₄ alkyl, C₁₋₄ haloalkyl, C₁₋₄ alkoxy, and C₁₋₄ haloalkoxy.

In certain embodiments (when one occurrence of R^{c} is -L¹-L²-R^{h}), R^{h} is C₆₋₁₀ aryl, which is optionally substituted with from 1-4 substituents independently selected from the group consisting of halo, C₁₋₄ alkyl, and C₁₋₄ haloalkyl.

In certain embodiments, R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

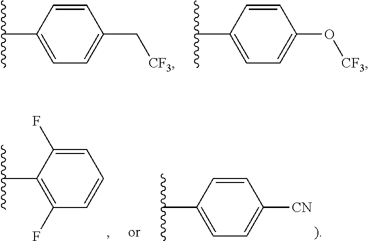 [SMILES: *c1ccc(CC(F)(F)F)cc1 ; *c1ccc(OC(F)(F)F)cc1 ; *c1c(F)cccc1F ; *c1ccc(C#N)cc1]

In certain of these embodiments, R^{h} is C₆ aryl, which is optionally substituted with from 1-2 substituents independently selected from the group consisting of halo, C₁₋₄ alkyl, and C₁₋₄ haloalkyl (e.g., R^{h} is unsubstituted phenyl; or R^{h} is

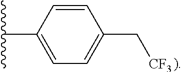 [SMILES: *c1ccc(CC(F)(F)F)cc1]

In one or more of the foregoing embodiments of R^{c}, each of the remaining R^{c} when present is independently halo or C₁₋₄ alkyl optionally substituted with R^{a}.

In some embodiments, wherein Y^{A2} is C₃₋₆(e.g., C₃, C₅, or C₆) cycloalkyl, which is substituted with from 1-4 (e.g., from 1-2) R^{b} (e.g., Y^{A2} is cyclopropyl, cyclopentyl, bicyclo[1.1.1]pentyl, or cyclohexyl, each of which is optionally substituted with from 1-2 R^{b}).

In certain of these embodiments, Y^{A2} is cyclohexyl which is optionally substituted with from 1-2 R^{b}.

In certain of these embodiments, one occurrence of R^{b} is at the ring carbon atom para to the point of attachment to Y^{A1}.

In certain embodiments, one occurrence of R^{b} is at the ring carbon atom meta to the point of attachment to Y^{A1},

In certain embodiments, one occurrence of R^{b} is at the ring carbon atom ortho to the point of attachment to Y^{A1}

In certain embodiments, Y^{A2} is C₇₋₁₀ cycloalkyl, which is optionally substituted with from 1-4 R^{b} (e.g., Y^{A2} is bicyclooctyl (e.g.,

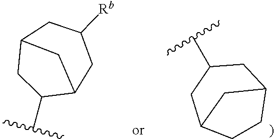

or spiroundecanyl (e.g., spiro[5,5]undecanyl such as

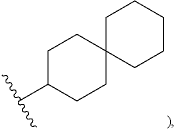 [SMILES: *C1CCC2(CCCCC2)CC1]

spirooctyl (e.g.,

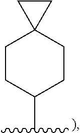 [SMILES: *C1CCC2(CC1)CC2]

each of which is further optionally substituted with from 1-3 R^{b}).

In some embodiments, Y^{A2} is heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b}.

In certain of these embodiments, Y^{A2} is heterocyclyl including from 5-12 (e.g., 5-10) ring atoms, wherein from 1-3 (e.g., 1 or 2) ring atoms are heteroatoms, each independently selected from the group consisting of N, N(H), N(R^{d}), O, and S(O)₀₋₂, and wherein the heterocyclyl ring is optionally substituted with from 1-4 independently selected R^{b} (e.g., Y^{A2} is pyrrolidinyl (e.g.,

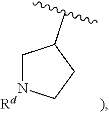 [SMILES: *C1CCNC1]

piperidinyl (e.g.,

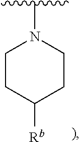

or tetrahydropyranyl (e.g.,

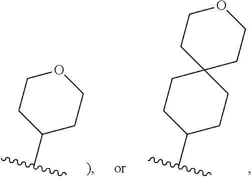 [SMILES: *C1CCOCC1 ; *C1CCC2(CCOCC2)CC1]

each of which is further optionally substituted with from 1-3 independently selected R^{b}).

In certain embodiments, Y^{A2} is heterocyclyl including from 5-6 (e.g., 5 or 6) ring atoms, wherein from 1-2 (e.g., 1 or 2) ring atoms are heteroatoms, each independently selected from the group consisting of N, N(H), N(R^{d}), O, and S(O)₀₋₂, and wherein the heterocyclyl ring is optionally substituted with from 1-4 independently selected R^{b} (e.g., Y^{A2} is pyrrolidinyl (e.g.,

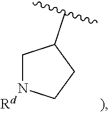 [SMILES: *C1CCNC1]

piperidinyl (e.g.,

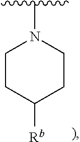

each of which is further optionally substituted with from 1-3 independently selected R^{b}).

As a non-limiting example of the foregoing embodiments, Y^{A2} is

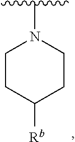

which is further optionally substituted with from 1-3 independently selected R^{b}.

In certain embodiments, each occurrence of R^{b} substituent of Y^{A2} is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

In certain embodiments, one occurrence of R^{b} substituent of Y^{A2} is C₁₋₁₀ alkyl which is optionally substituted with from 1-6 independently selected R^{a}.

In certain of these embodiments, one occurrence of R^{b} substituent of Y^{A2} is unsubstituted C₁₋₁₀ alkyl (e.g., C₂, C₃, C₄, C₅, C₆, or C₇₋₁₀).

In certain of the foregoing embodiments, one occurrence of R^{b} substituent of Y^{A2} is ethyl, propyl (e.g., n-propyl), butyl (e.g., n-butyl; or sec-butyl; or tert-butyl; or iso-butyl), or octyl (e.g., n-octyl) (e.g., butyl (e.g., n-butyl).

In certain embodiments, one occurrence of R^{b} substituent of Y^{A2} is C₁₋₁₀ alkyl which is substituted with from 1-6 independently selected R^{a}.

In certain of the foregoing embodiments, each occurrence of R^{a} is independently selected from —F, —Br, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

In certain embodiments, one occurrence of R^{b} is -L¹-L²-R^{h}. In certain of these embodiments, L¹ is a bond. In certain embodiments (when R^{b} is -L¹-L²-R^{h}), L² is a bond.

In certain embodiments (when R^{b} is -L¹-L²-R^{h}), R^{h} is C₃₋₆ cycloalkyl optionally substituted with from 1-4 substituents independently selected from the group consisting of halo, C₁₋₄ alkyl, and C₁₋₄ haloalkyl.

In certain embodiments (when R^{b} is -L¹-L²-R^{h}), R^{h} is heterocyclyl, wherein the heterocyclyl includes from 3-10 (e.g., 4, 5, 6, 7, 8, 9, or 10) ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl.

In certain embodiments (when R^{b} is -L¹-L²-R^{h}), R^{h} is C₆₋₁₀ aryl (e.g., C₆), which is optionally substituted with from 1-4 substituents independently selected from the group consisting of halo, C₁₋₄ alkyl, or C₁₋₄ haloalkyl (e.g., R^{h} is unsubstituted phenyl). In certain embodiments, one occurrence of R^{b} is —Cl or —F (e.g., —F); or wherein one occurrence of R^{b} is oxo or cyano.

In one or more of the foregoing embodiments of R^{b}, each remaining occurrence of R^{b} is independently selected from the group consisting of —Cl, —F, —Br, cyano, C₁₋₃ alkyl, and C₁₋₃ haloalkyl.

In certain embodiments, Y^{A2} is

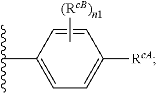

n1 is 0, 1, or 2; and each of R^{cA} and R^{cB} is an independently selected R^{c}.

In certain embodiments, Y^{A2} is

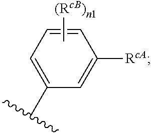

n1 is 0, 1, or 2; and each of R^{cA} and R^{cB} is an independently selected R^{c}.

In certain embodiments, Y^{A2} is

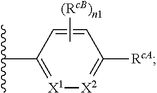

one of X¹ and X² is N; the other one of X¹ and X² is CH; n1 is 0, 1, or 2; and each of R^{cA} and R^{cB} is an independently selected R^{c}.

In certain embodiments, Y^{A2} is

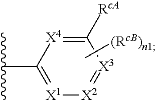

one of X¹, X², X³, and X⁴ is N; each of the remaining of X¹, X², X³, and X⁴ is CH; n1 is 0, 1, or 2; and each of R^{cA} and R^{cB} is an independently selected R^{c}.

In certain embodiments (when Y^{A2} is

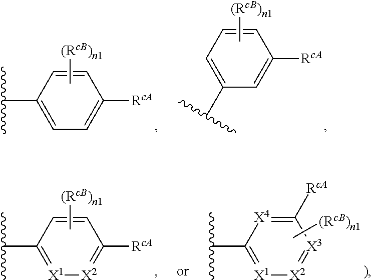

R^{cA} is as defined for R^{c} in any one of claims 124-133 of U.S. provisional application Ser. No. 62/861,714 which is incorporated herein by reference in its entirety.

In certain embodiments (when Y^{A2} is

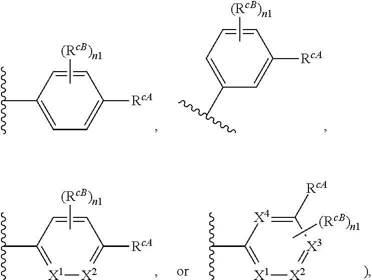

R^{cA} is as defined for R^{c} in any one of clauses 153-165 (e.g., 153, 154, 155, 156, 157, 159, 160, 161, 162, 163, 164, or 165).

In certain embodiments, R^{cA} is C₁₋₁₀ alkyl which is substituted with from 1-6 independently selected R^{a}. In certain of these embodiments, each R^{a} is independently selected from the group consisting —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl (e.g., each R^{a} is —F)).

In certain embodiments, R^{cA} is C₁₋₃ alkyl which is substituted with from 1-3 —F (e.g., R^{cA} is —CF₃). In certain embodiments, R^{cA} is unsubstituted C₁₋₁₀ alkyl (e.g., straight chain C₂, C₃, C₄, C₅, C₆, or C₇₋₁₀ alkyl).

In certain embodiments, R^{cA} is C₂₋₆ alkenyl; C₂₋₆ alkynyl; or —C(═O)(C₁₋₁₀ alkyl) (e.g., —C(═O)(C₃₋₁₀ alkyl) (e.g., —C(═O)CH₂CH₂CH₂CH₂CH₂CH₂CH₂)).

In certain embodiments, R^{cA} is selected from the group consisting of —SF₅; —S(O)₁₋₂(NR′R″) (e.g.,

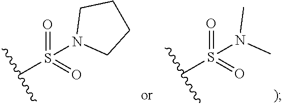

S(O)₁₋₂(C₁₋₄ alkyl); and S(O)₁₋₂(C₁₋₄ haloalkyl) (e.g., S(O)₂CF₃).

In certain embodiments, R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

In certain embodiments (when Y^{A2} is

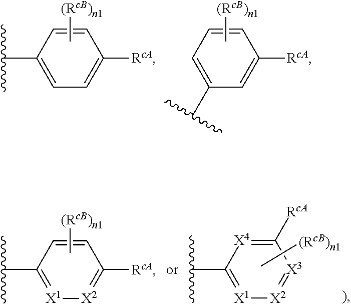

R^{cA} is as defined for R^{c} in any one of claims 134-143 of U.S. provisional application Ser. No. 62/861,714 which is incorporated herein by reference in its entirety.

In certain embodiments when Y^{A2} is

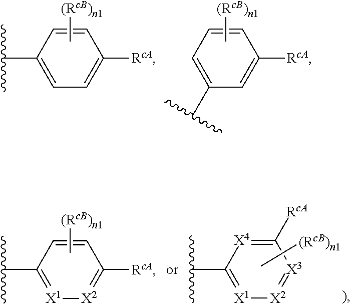

R^{cA} is as defined for R^{c} in any one of clauses 166-177 (e.g. R^{c} is -L¹-L²-R^{h}, such as R^{h}; and R^{h} is as defined in clause 175, clause 176, or clause 177).

In certain embodiments, R^{cA} is -L¹-L²-R^{h}, wherein: -L¹ is a bond, CH₂, or —CH₂CH₂; and -L² is a bond or —O—;

In certain of these embodiments, R^{h} is C₃₋₆ cycloalkyl optionally substituted with from 1-4 substituents independently selected from the group consisting of halo, C₁₋₄ alkyl, and C₁₋₄ haloalkyl (e.g.,

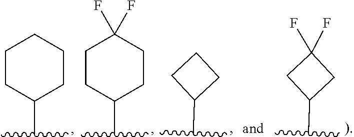

In certain embodiments, R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

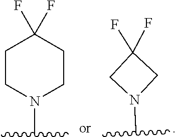 [SMILES: *N1CCC(F)(F)CC1 ; *N1CC(F)(F)C1]

In certain of these embodiments, R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

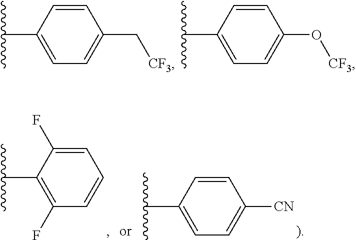

In certain embodiments (when Y^{A2} is

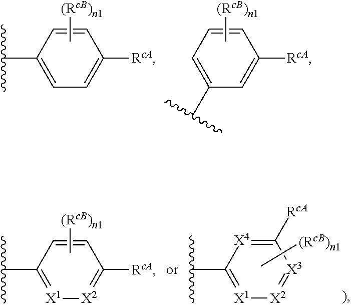

n1 is 0.

In certain other embodiments, n1 is 1 or 2. In certain of these embodiments, each R^{cB} is independently halo or C₁₋₄ alkyl optionally substituted with R^{a}.

In certain embodiments, Y^{A2} is

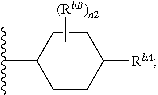

n2 is 0, 1, or 2; and each of R^{bA} and R^{bB} is an independently selected R^{b}.

In certain embodiments, Y^{A2} is

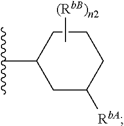

n2 is 0, 1, or 2; and each of R^{bA} and R^{bB} is an independently selected R^{b}.

In certain embodiments (when Y^{A2} is

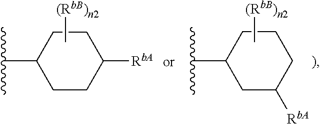

R^{bA} is as defined for R^{b} in claim 154 of U.S. provisional application Ser. No. 62/861,714 which is incorporated herein by reference in its entirety.

In certain embodiments (when Y^{A2} is

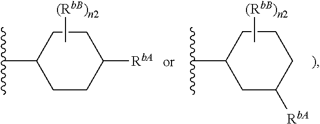

R^{bA} is as defined for R^{b} in clause 189.

In certain embodiments, R^{bA} is selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

In certain embodiments (when Y^{A2} is

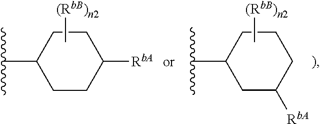

R^{bA} is as defined for R^{b} in any one of claims 155-159 of U.S. provisional application Ser. No. 62/861,714 which is incorporated herein by reference in its entirety.

In certain embodiments (when Y^{A2} is

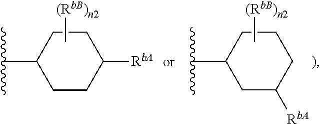

R^{bA} is as defined for R^{b} in any one of clauses 190-194 (e.g., 190, 191, 192, 193, or 194).

In certain embodiments, R^{bA} is C₁₋₁₀ alkyl which is optionally substituted with from 1-6 independently selected R^{a}.

In certain embodiments, R^{bA} is unsubstituted C₁₋₁₀ alkyl (e.g., straight-chain C₂, C₃, C₄, C₅, C₆, or C₇₋₁₀ alkyl).

In certain embodiments, R^{bA} is C₁₋₁₀ alkyl which is substituted with from 1-6 independently selected R^{a}, such as C₁₋₁₀ alkyl which is substituted with from 1-6 substituents each independently selected from the group consisting of: —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

In certain embodiments (when Y^{A2} is

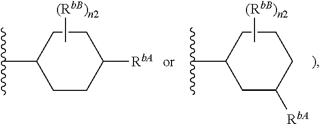

R^{bA} is as defined for R^{b} in any one of claims 160-165 of U.S. provisional application Ser. No. 62/861,714 which is incorporated herein by reference in its entirety.

In certain embodiments (when Y^{A2} is

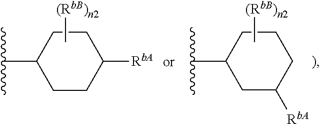

R^{bA} is as defined for R^{b} in any one of clauses 195-200 (e.g., clause 195, 196, 197, 198, 199, or 200).

In certain embodiments, R^{bA} is -L¹-L²-R^{h}, wherein: L¹ is a bond; L² is a bond or —O—; and

R^{h} is C₃₋₆cycloalkyl optionally substituted with from 1-4 substituents independently selected from the group consisting of halo, C₁₋₄ alkyl, and C₁₋₄ haloalkyl; or

R^{h} is C₆₋₁₀ aryl (e.g., C₆), which is optionally substituted with from 1-4 substituents independently selected from the group consisting of halo, C₁₋₆ alkyl, or C₁₋₄ haloalkyl (e.g., R^{h} is unsubstituted phenyl; or R^{h} is

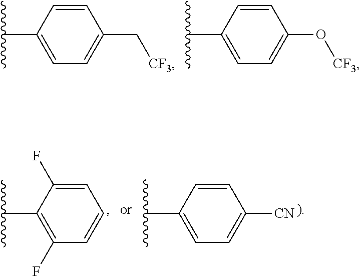

In certain embodiments (when Y^{A2} is

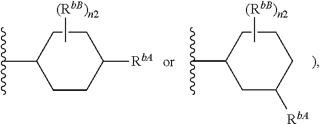

R^{bA} is as defined for R^{b} in claim 166 of U.S. provisional application Ser. No. 62/861,714 which is incorporated herein by reference in its entirety.

In certain embodiments, R^{bA} is —Cl or —F (e.g., F).

In certain embodiments (when Y^{A2} is

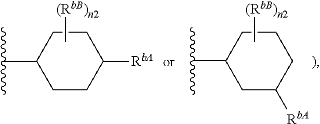

n2 is 0.

In certain other embodiments, n2 is 1 or 2. In certain of these embodiments, R^{bB} is independently selected from the group consisting of —Cl , —F, C₁₋₃ alkyl, and C₁₋₃ haloalkyl.

In some embodiments, A is C₁₋₁₀ alkyl, which is optionally substituted with from 1-6 independently selected R^{a}. In certain embodiments, A is C₂₋₁₀ (e.g., C₂, C₃, C₄, C₅, C₆, C₇, C₈, C₉, C₁₀) alkyl, which is optionally substituted with from 1-6 independently selected R^{a}.

In some embodiments, A is C₁₀₋₂₀ alkyl, which is optionally substituted with from 1-6 independently selected R^{a}. In certain embodiments, A is unsubstituted C₁₀₋₂₀ alkyl (e.g., C₁₀₋₁₂, C₁₃₋₁₅, C₁₆₋₁₈, C₁₉₋₂₀ alkyl). In certain embodiments, A is unsubstituted straight-chain C₁₀₋₂₀ alkyl (e.g., straight-chain C₁₀₋₁₂, C₁₃₋₁₅, C₁₆₋₁₈, C₁₉₋₂₀ alkyl).

### The Variable R⁶

In some embodiments, R⁶ is H. In some embodiments, R⁶ is C₁₋₃ alkyl.

### The Variable R^{N}

In some embodiments, each occurrence of R^{N} is independently H or C₁₋₃ alkyl.

In some embodiments, each occurrence of R^{N} is independently H.

### Non-Limiting Combinations

In some embodiments, the compound has the following formula:

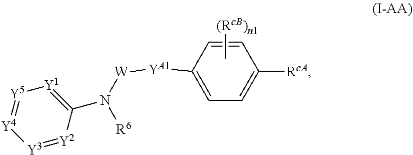

wherein n1 is 0, 1, or 2; and each of R^{cA} and R^{cB} is an independently selected R^{c}.

In some embodiments the compound has the following formula:

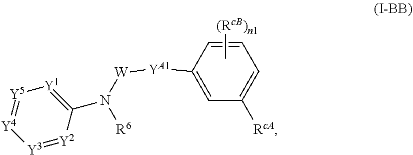

wherein n1 is 0, 1, or 2; and each of R^{cA} and R^{cB} is an independently selected R^{c}.

In some embodiments, the compound has the following formula:

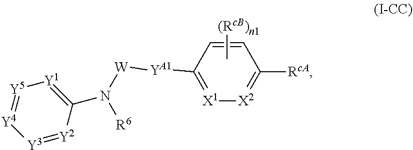

wherein one of X¹ and X² is N; the other one of X¹ and X² is CH; n1 is 0, 1, or 2; and each of R^{cA} and R^{cB} is an independently selected R^{c}.

In some embodiments, the compound has the following formula:

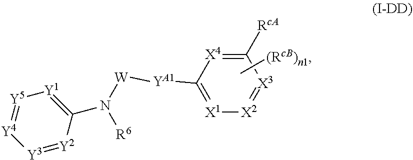

wherein one of X¹, X², X³, and X⁴ is N; each of the remaining of X¹, X², X³, X⁴ is CH; n1 is 0, 1, or 2; and each of R^{cA} and R^{cB} is an independently selected R^{c}.

In some embodiments, the compound has the following formula:

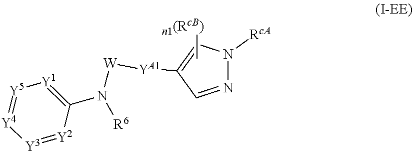

(e.g., R^{cA} is L¹-L²-R^{h}), wherein n1 is 0 or 1; and each of R^{cA} and R^{cB} is an independently selected R^{c}.

In some embodiments, the compound has one of the following formulae:

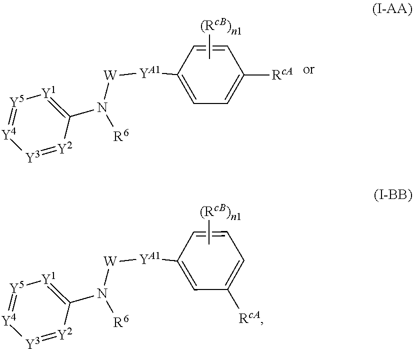

wherein:

n1 is 0, 1, or 2 (such as 0 or 1); each of R^{cA} and R^{cB} is an independently selected R^{c};

W is *C(═O)NR^{N}, such as *C(═O)NH; and

the

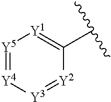

moiety is

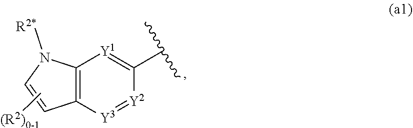

wherein R^{2′} is H or R².

In certain of these embodiments, the

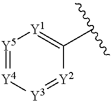

moiety is

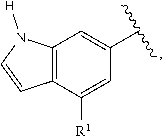

such as (a1-b) wherein R¹ is other than H (e.g., R¹ is halo or cyano).

In certain embodiments of Formulae (I-AA), (I-BB), (I-CC), (I-DD), and (I-EE), R^{cA} is as defined for R^{c} in any one of claims 124-133 of U.S. provisional application Ser. No. 62/861,714 which is incorporated herein by reference in its entirety; or wherein R^{cA} is as defined for R^{c} in any one of claims 134-143 of U.S. provisional application Ser. No. 62/861,714 which is incorporated herein by reference in its entirety.

In certain embodiments of Formulae (I-AA), (I-BB), (I-CC), (I-DD), and (I-EE), R^{cA} is as defined for R^{c} in any one of clauses 153-165 (e.g., 153, 154, 155, 156, 157, 159, 160, 161, 162, 163, 164, or 165); or R^{cA} is as defined for R^{c} in any one of clauses 166-177 (e.g. R^{c} is -L¹-L²-R^{h}, such as R^{h}; and R^{h} is as defined in clause 175, clause 176, or clause 177).

In certain embodiments of Formulae (I-AA), (I-BB), (I-CC), (I-DD), and (I-EE), R^{cA} is C₁₋₃ alkyl which is substituted with from 1-3 —F (e.g., R^{cA} is —CF₃).

In certain embodiments of Formulae (I-AA), (I-BB), (I-CC), (I-DD), and (I-EE), R^{cA} is unsubstituted C₁₋₁₀ alkyl (e.g., straight chain C₂, C₃, C₄, C₅, C₆, or C₇₋₁₀ alkyl); or

In certain embodiments of Formulae (I-AA), (I-BB), (I-CC), (I-DD), and (I-EE), R^{cA} is C₂₋₆ alkenyl, C₂₋₆ alkynyl, or —C(═O)(C₁₋₁₀ alkyl) (e.g., —C(═O)(C₃₋₁₀ alkyl) (e.g., —C(═O)CH₂CH₂CH₂CH₂CH₂CH₂CH₂));

In certain embodiments of Formulae (I-AA), (I-BB), (I-CC), (I-DD), and (I-EE), R^{cA} is selected from the group consisting of —SF₅, —S(O)₁₋₂(NR′R″) (e.g.,

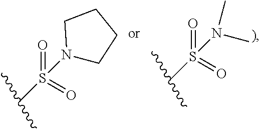

S(O)₁₋₂(C₁₋₄ alkyl), and S(O)₁₋₂(C₁₋₄ haloalkyl) (e.g., S(O)₂CF₃).

In certain embodiments of Formulae (I-AA), (I-BB), (I-CC), (I-DD), and (I-EE), R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

In certain embodiments of Formulae (I-AA), (I-BB), (I-CC), (I-DD), and (I-EE), R^{cA} is -L¹-L²-R^{h}.

In certain of these embodiments, -L¹ is a bond. In certain other embodiments, -L¹ is CH₂, or —CH₂CH₂. In certain embodiments, -L² is a bond or —O—.

In certain embodiments, R^{h} is C₃₋₆ cycloalkyl optionally substituted with from 1-4 substituents independently selected from the group consisting of halo, C₁₋₄ alkyl, and C₁₋₄ haloalkyl (e.g.,

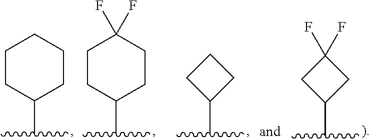

In certain embodiments, R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

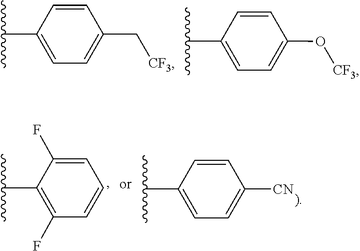

In certain embodiments of Formulae (I-AA), (I-BB), (I-CC), (I-DD), and (I-EE), n1 is 0. In certain other embodiments, n1 is 1. In certain of these embodiments, each R^{cB} is independently halo or C₁₋₄ alkyl optionally substituted with R^{a}.

In some embodiments, the compound has the following formula:

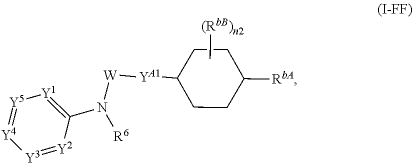

wherein n2 is 0, 1, or 2; and each of R^{bA} and R^{bB} is an independently selected R^{b}.

In some embodiments, the compound has the following formula:

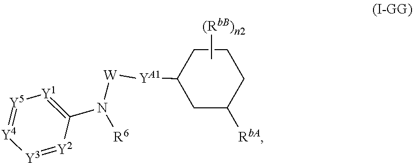

wherein n2 is 0, 1, or 2; and each of R^{bA} and R^{bB} is an independently selected R^{b}.

In some embodiments, the compound has the following formula:

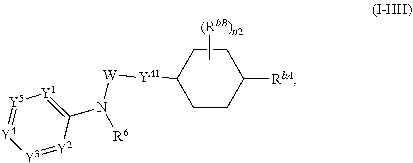

wherein n2 is 0, 1, or 2; and each of R^{bA} and R^{bB} is an independently selected R^{b}.

In certain embodiments of Formulae (I-FF), (I-GG), and (I-HH), R^{bA} is as defined in any one of claims 155-159 of U.S. provisional application Ser. No. 62/861,714 which is incorporated herein by reference in its entirety.

In certain embodiments of Formulae (I-FF), (I-GG), and (I-HH), R^{bA} is as defined in any one of clauses 190-194 (e.g., 190, 191, 192, 193, or 194).

In certain of these embodiments, R^{bA} is unsubstituted C₁₋₁₀ alkyl (e.g., straight-chain C₂, C₃, C₄, C₅, C₆, or C₇₋₁₀ alkyl).

In certain other embodiments, R^{bA} is C₁₋₁₀ alkyl which is substituted with from 1-6 independently selected R^{a}, such as C₁₋₁₀ alkyl which is substituted with from 1-6 substituents each independently selected from the group consisting of: —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

In certain embodiments of Formulae (I-FF), (I-GG), and (I-HH), R^{bA} is as defined in any one of claims 160-165 of U.S. provisional application Ser. No. 62/861,714 which is incorporated herein by reference in its entirety.

In certain embodiments of Formulae (I-FF), (I-GG), and (I-HH), R^{bA} is as defined in any one of clauses 195-200 (e.g., 195, 196, 197, 198, 199, or 200).

In certain embodiments, R^{bA} is -L¹-L²-R^{h}, wherein: L¹ is a bond; and/or L² is a bond or —O—; and/or

R^{h} is C₃₋₆ cycloalkyl optionally substituted with from 1-4 substituents independently selected from the group consisting of halo, C₁₋₄ alkyl, and C₁₋₄ haloalkyl; and/or

R^{h} is C₆₋₁₀ aryl (e.g., C₆), which is optionally substituted with from 1-4 substituents independently selected from the group consisting of halo, C₁₋₄ alkyl, or C₁₋₄ haloalkyl (e.g., R^{h} is unsubstituted phenyl; or R^{h} is

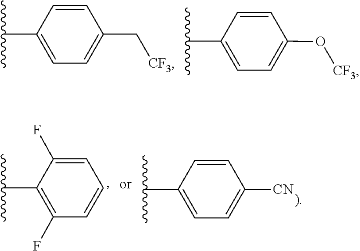

In certain embodiments of Formulae (I-FF), (I-GG), and (I-HH), R^{bA} is as defined in claim 166 of U.S. provisional application Ser. No. 62/861,714 which is incorporated herein by reference in its entirety.

In certain embodiments of Formulae (I-FF), (I-GG), and (I-HH), R^{b} is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

In certain embodiments of Formulae (I-FF), (I-GG), and (I-HH), n2 is 0.

In certain other embodiments, n2 is 1 or 2. In certain of these embodiments, each R^{bB} is independently —F, —Cl , or C₁₋₃ alkyl.

In some embodiments, the compound has the following formula:

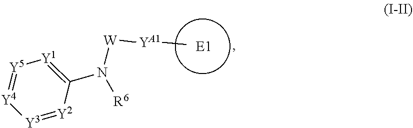

wherein ring E1 is C₇₋₁₀ cycloalkyl, which is optionally substituted with from 1-4 R^{b} (e.g., Y^{A2} is bicyclooctyl (e.g.,

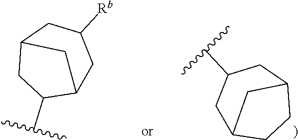

or spiroundecanyl (e.g., spiro[5,5]undecanyl such as

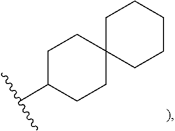 [SMILES: *C1CCC2(CCCCC2)CC1]

each of which is further optionally substituted with from 1-3 R^{b}).

In certain of these embodiments, R^{b} is as defined in claim 154 of U.S. provisional application Ser. No. 62/861,714 which is incorporated herein by reference in its entirety.

In certain embodiments of Formula (I-II), R^{b} is as defined in clause 189.

In certain embodiments of Formula (I-II), R^{b} substituent of ring E1 is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

In certain embodiments of Formulae (I-AA), (I-BB), (I-CC), (I-DD), (I-EE), (I-FF), (I-GG), (I-HH), and (I-II, Y^{A1} is a bond.

In certain embodiments of Formulae (I-AA), (I-BB), (I-CC), (I-DD), (I-EE), (I-FF), (I-GG), (I-HH), and (I-II), Y^{A1} is CH₂ or C(═O).

In certain embodiments of Formulae (I-AA), (I-BB), (I-CC), (I-DD), (I-EE), (I-FF), (I-GG), (I-HH), and (I-II), Y^{A1} is C₁₋₄ alkylene, optionally substituted with from 1-2 independently selected R^{a}. As non-limiting examples, Y^{A1} can be: —CH₂—, —CH₂CH₂—, —CH₂CH₂CH₂—, —CH(CF₃), —CH₂CH(OH)—,

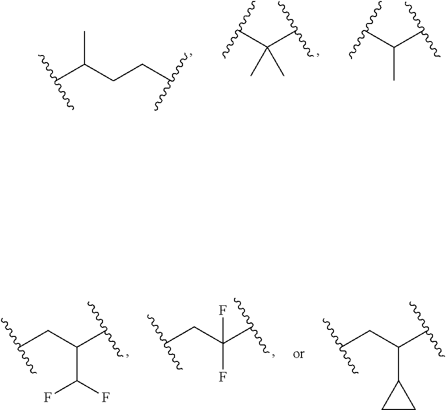

In some embodiments, the compound has the following formula:

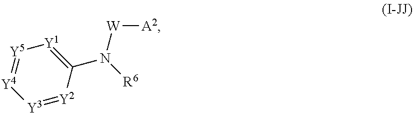

wherein A² is C₁₋₂₀ alkyl, which is optionally substituted with from 1-6 independently selected R^{a}.

In certain embodiments of Formula (I-JJ), A² is C₈₋₂₀ (e.g., C₈, C₉, C₁₀, C₁₁₋₁₃, C₁₄₋₁₆, C₁₇₋₁₉, or C₂₀) alkyl, which is optionally substituted with from 1-6 independently selected R^{a}. In certain embodiments, A² is unsubstituted C₈₋₂₀ (e.g., C₈, C₉, C₁₀, C₁₁₋₁₃, C₁₄₋₁₆, C₁₇₋₁₉, or C₂₀) alkyl. In certain embodiments, A² is unsubstituted C₁₀₋₂₀ (e.g., C₁₀, C₁₁₋₁₃, C₁₄₋₁₆, C₁₇₋₁₉, or C₂₀) alkyl. As a non-limiting example, A² can be straight-chain C₁₀₋₂₀ (e.g., C₁₀, C₁₁₋₁₃, C₁₄₋₁₆, C₁₇₋₁₉, or C₂₀) alkyl.

In certain embodiments of Formulae (I-AA), (I-BB), (I-CC), (I-DD), (I-EE), (I-FF), (I-GG), (I-HH), (I-II), and (I-JJ), W is *C(═O)NR^{N}. In certain embodiments of Formulae (I-AA), (I-BB), (I-CC), (I-DD), (I-EE), (I-FF), (I-GG), (I-HH), (I-II), and (I-JJ), W is *C(═O)NH or *C(═O)N(C₁₋₃ alkyl). In certain embodiments of Formulae (I-AA), (I-BB), (I-CC), (I-DD), (I-EE), (I-FF), (I-GG), (I-HH), (I-II), and (I-JJ), W is *C(═O)NH.

In certain embodiments of Formulae (I-AA), (I-BB), (I-CC), (I-DD), (I-EE), (I-FF), (I-GG), (I-HH), (I-II), and (I-JJ), W is *S(O)₁₋₂NR^{N}. In certain embodiments of Formulae (I-AA), (I-BB), (I-CC), (I-DD), (I-EE), (I-FF), (I-GG), (I-HH), (I-II), and (I-JJ), W is *S(O)₂NR^{N} (e.g., *S(O)₂NH).

In certain embodiments of Formulae (I-AA), (I-BB), (I-CC), (I-DD), (I-EE), (I-FF), (I-GG), (I-HH), (I-II), and (I-JJ), W is *C(═NR^{N})NR^{N} (e.g., C(═NCN)NH).

In certain embodiments of Formulae (I-AA), (I-BB), (I-CC), (I-DD), (I-EE), (I-FF), (I-GG), (I-HH), (I-II), and (I-JJ), W is

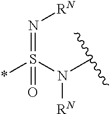

(e.g., each R^{N} is H).

In certain embodiments of Formulae (I-AA), (I-BB), (I-CC), (I-DD), (I-EE), (I-FF), (I-GG), (I-HH), (I-II), and (I-JJ), W is

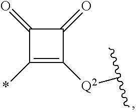

In certain of these embodiments, Q² is NR^{N}. As non-limiting examples, Q² is NH or N(C₁₋₃ alkyl) (e.g., NH).

In certain embodiments of Formulae (I-AA), (I-BB), (I-CC), (I-DD), (I-EE), (I-FF), (I-GG), (I-HH), (I-11), and (I-JJ), W is -Q¹-Q² (e.g., Q¹ is heteroarylene including 6 ring atoms, wherein from 1-3 (e.g., 1-2) ring atoms are ring nitrogen atoms, and wherein the heteroarylene ring is optionally substituted with from 1-2 independently selected R^{q1}).

In certain of these embodiments, Q¹ is selected from the group consisting of:

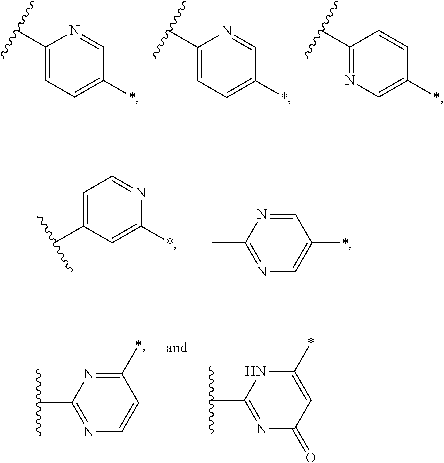

each of which is optionally substituted with 1-2 independently selected R^{q1}, wherein the asterisk denotes point of attachment of Q² (e.g.,

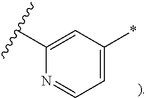 [SMILES: *c1ccnc(*)c1]

In certain embodiments of Formulae (I-AA), (I-BB), (I-CC), (I-DD), (I-EE), (I-FF), (I-GG), (I-HH), (I-II), and (I-JJ) (when W is -Q¹-Q²), Q² is a bond.

In certain embodiments of Formulae (I-AA), (I-BB), (I-CC), (I-DD), (I-EE), (I-FF), (I-GG), (I-HH), (I-II, and (I-JJ) (when W is -Q¹-Q²), Q² is —O—, —NH—, or —S(O)₀₋₂ (e.g., Q² is —O—; or Q² is —NH—; or Q² is —S(O)₂—).

In some embodiments, the compound has Formula (I-KK):

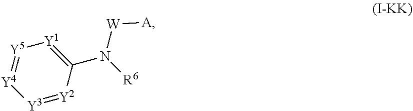

wherein A is H; and W is selected from the group consisting of: C₈₋₁₀ bicyclic arylene, which is optionally substituted with from 1-4 R^{c}; and heteroarylene including from 8-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-3 independently selected R^{c}.

In certain of these embodiments, W is heteroarylene including from 9-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-2 independently selected R^{c}.

In certain of these embodiments, W is selected from the group consisting of quinolinylene, isoquinolinylene, and quinazolinylene, each of which is optionally substituted with from 1-2 independently selected R^{c}.

By way of non-limiting examples, W can be

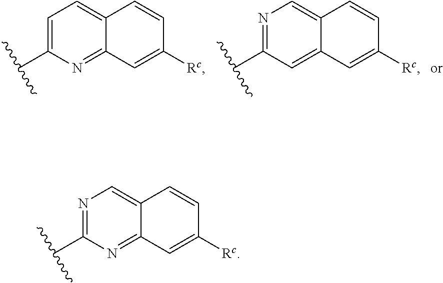

In certain embodiments of Formula (I-KK), one occurrence of R^{c} is C₁₋₁₀ alkyl which is substituted with from 1-6 independently selected R^{a} (e.g., —CF₃).

In certain embodiments of Formula (I-KK), one occurrence of R^{c} is halo (e.g., —Cl or F).

In certain embodiments of Formula (I-KK), one occurrence of R^{c} is -L¹-L²-R^{h}.

In certain of these embodiments, one occurrence R^{c} is R^{h}, wherein R^{h} is C₃₋₆ cycloalkyl optionally substituted with from 1-4 substituents independently selected from the group consisting of halo, C₁₋₄ alkyl, and C₁₋₄ haloalkyl (e.g.,

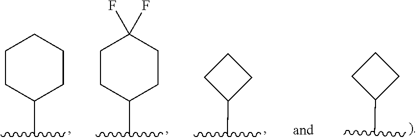

In certain embodiments of Formulae (I-AA), (I-BB), (I-CC), (I-DD), (I-EE), (I-FF), (I-GG), (I-HH), (I-II), (I-JJ), and (I-KK), the

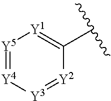

moiety is

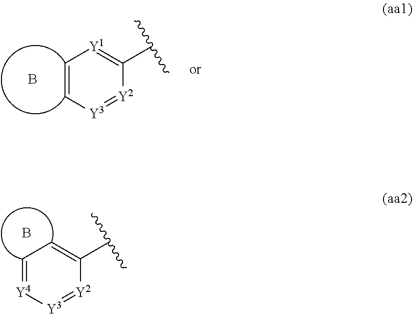

wherein ring B is a ring (e.g., monocyclic ring, bicyclic ring, or tricyclic ring) including from 4-15 (e.g., 5-12 (e.g., 5-10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

In certain embodiments of Formulae (I-AA), (I-BB), (I-CC), (I-DD), (I-EE), (I-FF), (I-GG), (I-HH), (I-II), (I-JJ), and (I-K), the

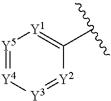

moiety is

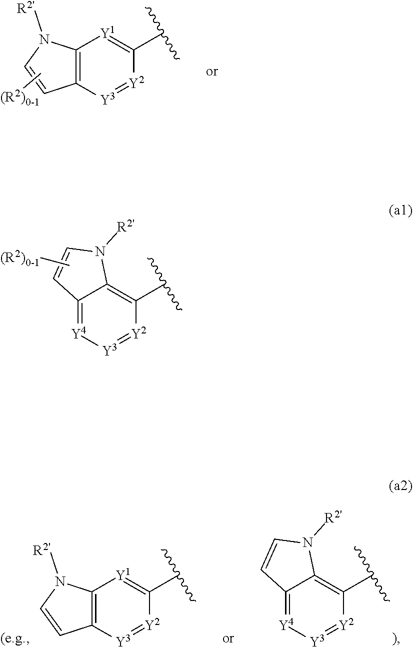

wherein R^{2′} is H or R² (e.g., R^{2′} is H).

In certain embodiments of Formulae (I-AA), (I-BB), (I-CC), (I-DD), (I-EE), (I-FF), (I-GG), (I-HH), (I-II), (I-JJ), and (I-KK), the

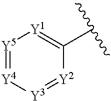

moiety is

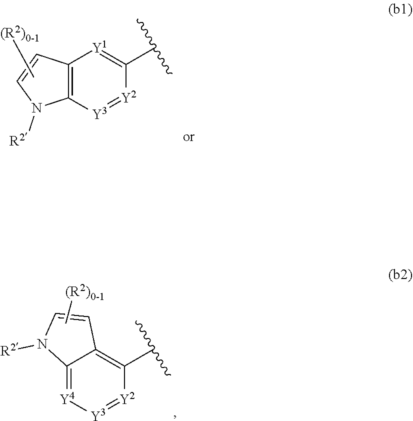

wherein R^{2′} is H or R² (e.g.,

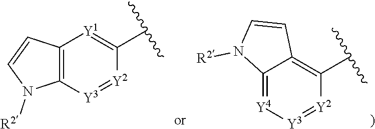

### (e.g., R^{2′} is H).

In certain embodiments of Formulae (I-AA), (I-BB), (I-CC), (I-DD), (I-EE), (I-FF), (I-GG), (I-HH), (I-II), (I-JJ), and (I-KK), the

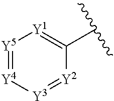

moiety is

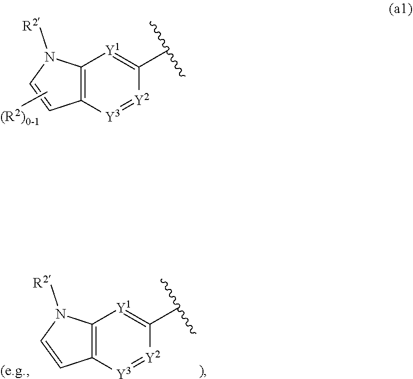

wherein R^{2′} is H or R² (e.g., R^{2′} is H).

In certain embodiments of Formulae (I-AA), (I-BB), (I-CC), (I-DD), (I-EE), (I-FF), (I-GG), (I-HH), (I-II), (I-JJ), and (I-KK), the

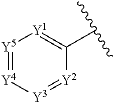

moiety is

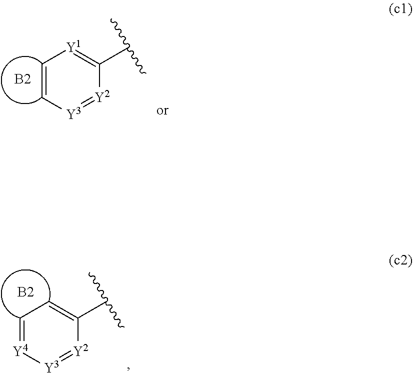

wherein B2 is an aromatic ring including 5 ring atoms, wherein from 1-2 (e.g., 2) ring atoms are heteroatoms each independently selected from the group consisting of N, N(H), N(R^{d}), O, and S(O)₀₋₂, provided that B2 is other than pyrrolyl; and wherein the ring is optionally substituted with from 1-4 independently selected R².

In certain of these embodiments, B2 is

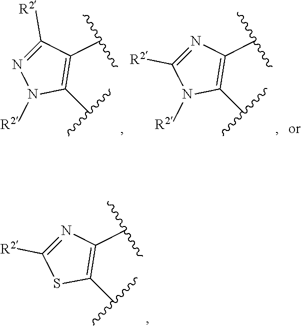

wherein each R^{2′} is independently H or R² (e.g.,

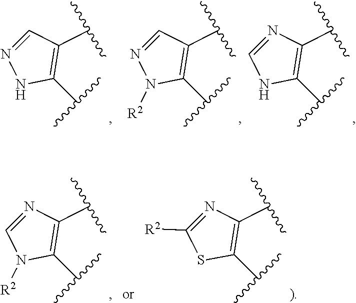

In certain embodiments of Formulae (I-AA), (I-BB), (I-CC), (I-DD), (I-EE), (I-FF), (I-GG), (I-HH), (I-II), (I-JJ), and (I-KK), the

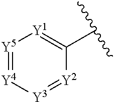

moiety is

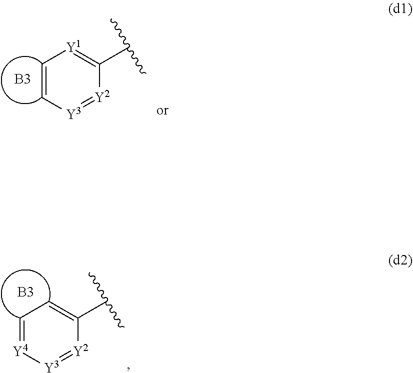

or wherein B3 is selected from the group consisting of:

a) a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

b) a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain of these embodiments, B3 is a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R² (e.g.,

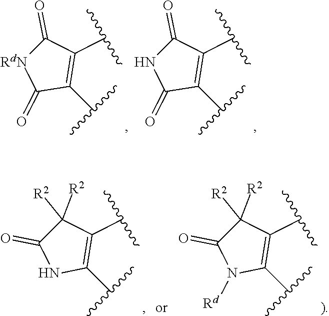

In certain embodiments, B3 is non-aromatic ring including 5 ring atoms, wherein from 0-1 ring atoms is a heteroatom selected from the group consisting of N, N(H), N(R^{d}), O, and S(O)₀₋₂; wherein the ring is optionally substituted with from 1-2 independently selected R² (e.g.,

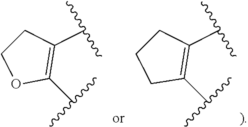 [SMILES: *C1=C(*)OCC1 ; *C1=C(*)CCC1]

In certain embodiments, B3 is a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

In certain embodiments, B3 is a spirocyclic bicyclic ring including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R² (e.g., B3 is

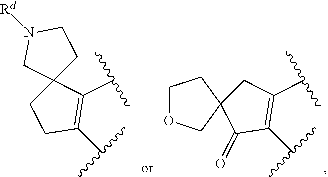

each of which is further optionally substituted with from 1-2 independently selected R²).

In certain embodiments of Formulae (I-AA), (I-BB), (I-CC), (I-DD), (I-EE), (I-FF), (I-GG), (I-HH), (I-II, (I-JJ), and (I-KK), the

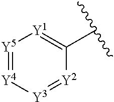

moiety is

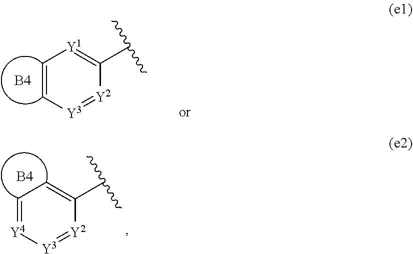

wherein B4 is an aromatic ring including 6 ring atoms, wherein from 0-2 ring atoms are heteroatoms each independently selected from the group consisting of N, N(H), and N(R^{d}); and wherein the ring is optionally substituted with from 1-4 independently selected R².

In certain embodiments of Formulae (I-AA), (I-BB), (I-CC), (I-DD), (I-EE), (I-FF), (I-GG), (I-HH), (I-II, (I-JJ), and (I-KK), when the

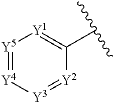

moiety is (aa1), (a1), (b1), (c1), (d1), or (e1), each of Y¹, Y², and Y³ is an independently selected CR¹; and

when the

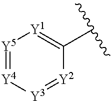

moiety is (aa2), (a2), (b2), (c2), (d2), or (e2), each of Y², Y³, and Y⁴ is an independently selected CR¹.

In certain embodiments of Formulae (I-AA), (I-BB), (I-CC), (I-DD), (I-EE), (I-FF), (I-GG), (I-HH), (I-II, (I-JJ), and (I-KK), when the

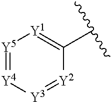

moiety is (aa1), (a1), (b1), (c1), (d1), or (e1), one of Y¹, Y², and Y³ is N; and each of the remaining of Y¹, Y², and Y³ is an independently selected CR¹; and

when the

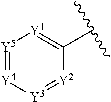

moiety is (aa2), (a2), (b2), (c2), (d2), or (e2), one of Y², Y³, and Y⁴ is N; and each of the remaining of Y², Y³, and Y⁴ is an independently selected CR¹.

In certain embodiments of Formulae (I-AA), (I-BB), (I-CC), (I-DD), (I-EE), (I-FF), (I-GG), (I-HH) (I-II), (I-JJ), and (I-KK), the

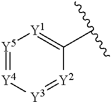

moiety is selected from the group consisting of:

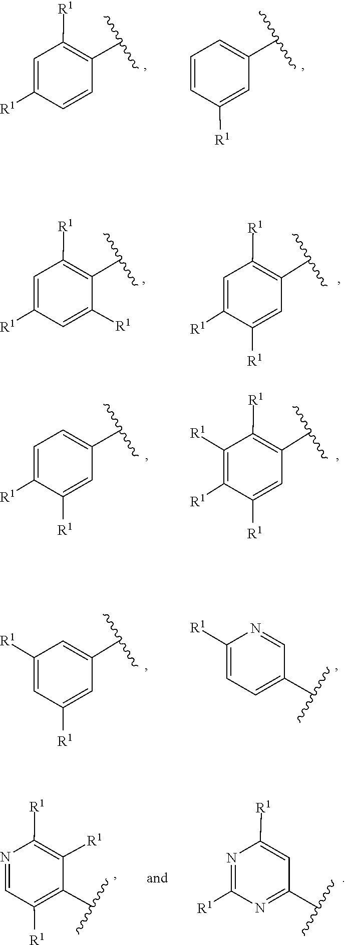

In certain embodiments of Formulae (I-AA), (I-BB), (I-CC), (I-DD), (I-EE), (I-FF), (I-GG), (I-HH), (I-II), (I-JJ), and (I-KK), the

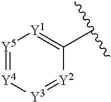

moiety is

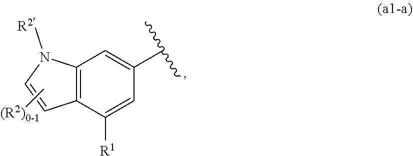

wherein R^{2′} is H or R².

In certain embodiments of Formulae (I-AA), (I-BB), (I-CC), (I-DD), (I-EE), (I-FF), (I-GG), (I-HH), (I-II, (I-JJ), and (I-KK), the

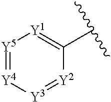

moiety is

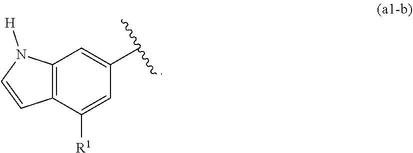

In certain embodiments of Formulae (I-AA), (I-BB), (I-CC), (I-DD), (I-EE), (I-FF), (I-GG), (I-HH), (I-II), (I-JJ), and (I-KK), the

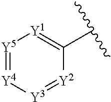

moiety is

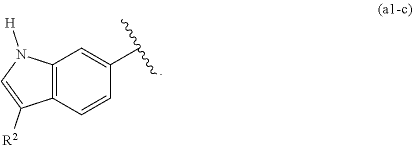

In certain embodiments of Formulae (I-AA), (I-BB), (I-CC), (I-DD), (I-EE), (I-FF), (I-GG), (I-HH), (I-II), (I-JJ), and (I-KK) the

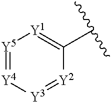

moiety is

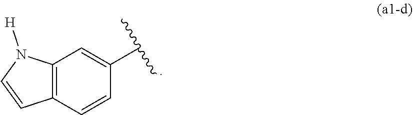 [SMILES: *c1ccc2cc[nH]c2c1]

In certain embodiments of Formulae (I-AA), (I-BB), (I-CC), (I-DD), (I-EE), (I-FF), (I-GG), (I-HH), (I-II), (I-JJ), and (I-KK), the

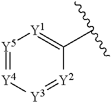

moiety is

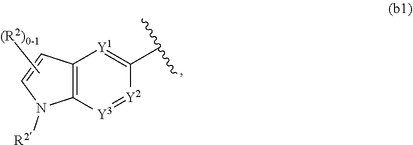

wherein R^{2′} is H or R² (e.g., R^{2′} is H).

In certain embodiments of Formulae (I-AA), (I-BB), (I-CC), (I-DD), (I-EE), (I-FF), (I-GG), (I-HH), (I-II), (I-JJ), and (I-KK), the

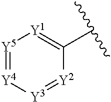

moiety is

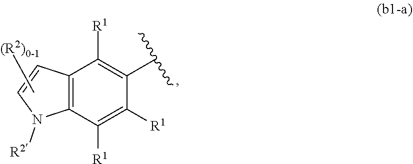

wherein R^{2′} is H or R².

In certain embodiments of Formulae (I-AA), (I-BB), (I-CC), (I-DD), (I-EE), (I-FF), (I-GG), (I-HH), (I-II), (I-JJ), and (I-KK), the

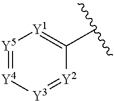

moiety is

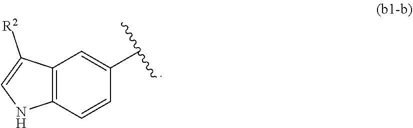

In certain embodiments of Formulae (I-AA), (I-BB), (I-CC), (I-DD), (I-EE), (I-FF), (I-GG), (I-HH), (I-II), (I-JJ), and (I-KK), the

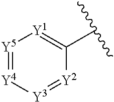

moiety is

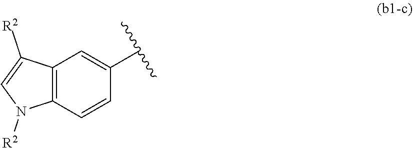

In certain embodiments of Formulae (I-AA), (I-BB), (I-CC), (I-DD), (I-EE), (I-FF), (I-GG), (I-HH), (I-II), (I-JJ), and (I-KK), the

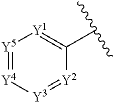

moiety is

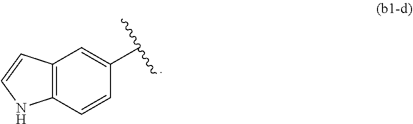 [SMILES: *c1ccc2[nH]ccc2c1]

In certain embodiments of Formulae (I-AA), (I-BB), (I-CC), (I-DD), (I-EE), (I-FF), (I-GG), (I-HH) (I-II, (I-JJ), and (I-KK), each occurrence of R¹ is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —NR^{e}R^{f}; —OH; oxo; —S(O)₁₋₂(NR′R″); —C(═O)(C₁₋₄ alkyl); -C(═O)O(C₁₋₄ alkyl); —C(═O)OH; —C(═O)N(R′)(R″); and -L³-L⁴-Rⁱ.

In certain embodiments of Formulae (I-AA), (I-BB), (I-CC), (I-DD), (I-EE), (I-FF), (I-GG), (I-HH), (I-II, (I-JJ), and (I-KK), R¹ is as defined in any one of claims 59-64 of U.S. provisional application Ser. No. 62/861,714 which is incorporated herein by reference in its entirety.

In certain embodiments of Formulae (I-AA), (I-BB), (I-CC), (I-DD), (I-EE), (I-FF), (I-GG), (I-HH) (I-II), (I-JJ), and (I-KK), each R¹ is H.

In certain embodiments of Formulae (I-AA), (I-BB), (I-CC), (I-DD), (I-EE), (I-FF), (I-GG), (I-HH) (I-H), (I-JJ), and (I-KK), one occurrence of R¹ that is not taken together with the atom to which it is attached in ring formation is selected from the consisting of: halo, cyano, —C(═O)O(C₁₋₄ alkyl), —C(═O)OH, and C₁₋₆ alkyl optionally substituted with 1-2 R^{a}; and each remaining R¹ that is not taken together with the atom to which it is attached in ring formation is H.

In certain embodiments of Formulae (I-AA), (I-BB), (I-CC), (I-DD), (I-EE), (I-FF), (I-GG), (I-HH) (I-H), (I-JJ), and (I-KK), one occurrence of R¹ that is not taken together with the atom to which it is attached in ring formation is —Rⁱ; and each remaining R¹ that is not taken together with the atom to which it is attached in ring formation is H.

In certain embodiments of Formulae (I-AA), (I-BB), (I-CC), (I-DD), (I-EE), (I-FF), (I-GG), (I-HH), (I-II), (I-JJ), and (I-KK), (when the

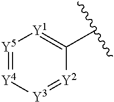

moiety is selected from the group consisting of:

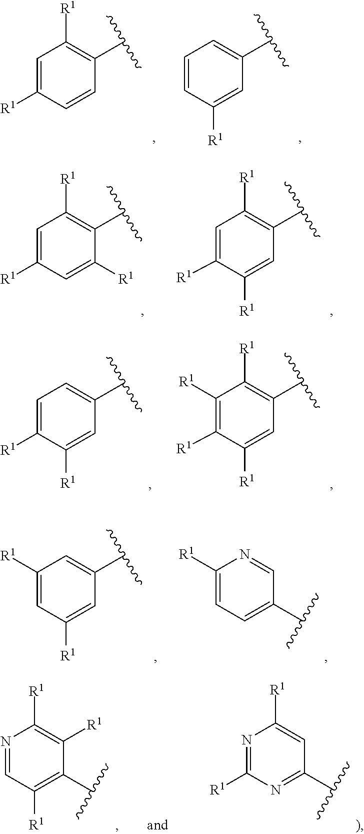

each R¹ is other than H.

In certain embodiments of Formulae (I-AA), (I-BB), (I-CC), (I-DD), (I-EE), (I-FF), (I-GG), (I-HH), (I-II), (I-JJ), and (I-KK), each occurrence of R² is as defined in any one of claims 66-85 of U.S. provisional application Ser. No. 62/861,714 which is incorporated herein by reference in its entirety.

In certain embodiments of Formulae (I-AA), (I-BB), (I-CC), (I-DD), (I-EE), (I-FF), (I-GG), (I-HH), (I-II), (I-JJ), and (I-KK), each occurrence of R² is independently selected from the group consisting of halo, cyano, —C(═O)O(C₁₋₄ alkyl), —C(═O)OH, and C₁₋₆ alkyl optionally substituted with 1-2 R^{a}.

In some embodiments the compound has Formula (I-LL):

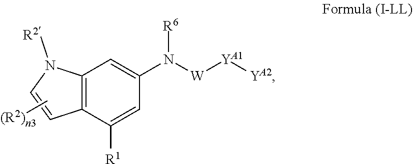

wherein: R^{2′} is H or R²; and n3 is 0 or 1.

In certain of these embodiments, n3=0. In certain other embodiments, n3=1. In certain embodiments, R^{2′} is H.

In certain embodiments of Formula (I-LL), R¹ is H. In certain other embodiments, R¹ is other than H. In certain of these embodiments, R¹ is selected from the consisting of: halo, cyano, —C(═O)O(C₁₋₄alkyl), —C(═O)OH, and C₁₋₆ alkyl optionally substituted with 1-2 R^{a}.

In certain embodiments, R¹ is other than H; n3 is 0; and R^{2′} is H. In certain embodiments, R¹ is other than H; n3 is 1; and R² is H.

In certain embodiments of Formula (I-LL), R² is independently selected from the group consisting of halo, cyano, —C(═O)O(C₁₋₄ alkyl), —C(═O)OH, and C₁₋₆ alkyl optionally substituted with 1-2 R^{a}.

In certain embodiments of Formula (I-LL), W is *C(═O)NR^{N}. In certain of these embodiments, W is *C(═O)NH.

In certain embodiments of Formula (I-LL), Y^{A1} is a bond.

In certain other embodiments, Y^{A1} is C₁₋₆ alkylene which is optionally substituted with from 1-2 R^{a}. For example, Y^{A1} can be —CH₂—, —CH₂CH₂—, —CH₂CH₂CH₂—, —CH(CF₃)—, —CH₂CH(OH)—,

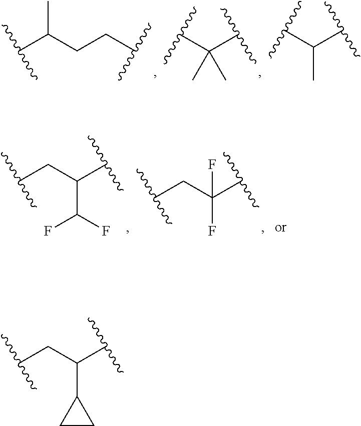

(e.g., Y^{A1} is CH₂).

In certain embodiments of Formula (I-LL), Y^{A2} is

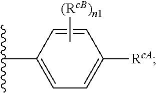

n1 is 0, 1, or 2; and each of R^{cA} and R^{cB} is an independently selected R^{c}.

In certain embodiments of Formula (I-LL), Y^{A2} is

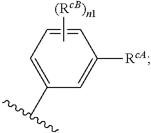

n1 is 0, 1, or 2; and each of R^{cA} and R^{cB} is an independently selected R^{c}.

In certain embodiments of Formula (I-LL), Y^{A2} is

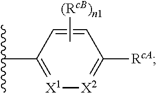

one of X¹ and X² is N; the other one of X¹ and X² is CH; n1 is 0, 1, or 2; and each of R^{cA} and R^{cB} is an independently selected R^{c} (e.g., X² is N).

In certain embodiments of Formula (I-LL), Y^{A2} is

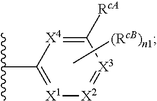

one of X¹, X², X³, and X⁴ is N; each of the remaining of X¹, X², X³, and X⁴ is CH; n1 is 0, 1, or 2; and each of R^{cA} and R^{cB} is an independently selected R^{c} (e.g., X² is N).

In certain embodiments of Formula (I-LL), R^{cA} is C₁₋₃ alkyl which is substituted with from 1-3 —F (e.g., R^{cA} is —CF₃).

In certain embodiments of Formula (I-LL), R^{cA} is unsubstituted C₁₋₁₀ alkyl (e.g., straight chain C₂, C₃, C₄, C₅, C₆, or C₇₋₁₀ alkyl).

In certain embodiments of Formula (I-LL), R^{cA} is C₂₋₆ alkenyl, C₂₋₆ alkynyl, or —C(═O)(C₁₋₁₀ alkyl) (e.g., —C(═O)(C₃₋₁₀ alkyl) (e.g., —C(═O)CH₂CH₂CH₂CH₂CH₂CH₂CH₂));

In certain embodiments of Formula (I-LL), R^{cA} is selected from the group consisting of —SF₅, —S(O)₁₋₂(NR′R″) (e.g.,

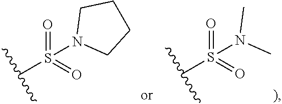

S(O)₁₋₂(C₁₋₄ alkyl), and S(O)₁₋₂(C₁₋₄ haloalkyl) (e.g., S(O)₂CF₃).

In certain embodiments of Formula (I-LL), R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

In certain embodiments of Formula (I-LL), R^{cA} is -L¹-L²-R^{h}.

In certain of these embodiments, -L¹ is a bond. In certain other embodiments, -L¹ is CH₂, or —CH₂CH₂. In certain embodiments, -L² is a bond or —O—.

In certain embodiments, R^{h} is C₃₋₆ cycloalkyl optionally substituted with from 1-4 substituents independently selected from the group consisting of halo, C₁₋₄ alkyl, and C₁₋₄ haloalkyl (e.g.,

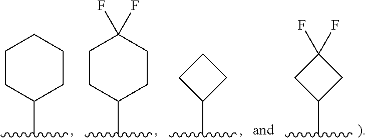

In certain embodiments, R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

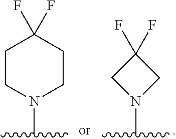 [SMILES: *N1CCC(F)(F)CC1 ; *N1CC(F)(F)C1]

In certain embodiments, R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

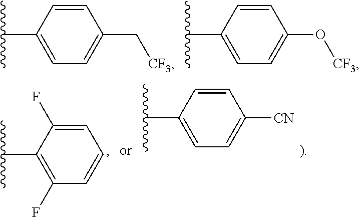

In certain embodiments of Formula (I-LL), n1 is 0. In certain other embodiments, n1 is 1. In certain of these embodiments, each R^{cB} is independently halo or C₁₋₄ alkyl optionally substituted with R^{a}.

In certain embodiments of Formula (I-LL), R⁶ is H.

In some embodiments, the compound has Formula (I-MM):

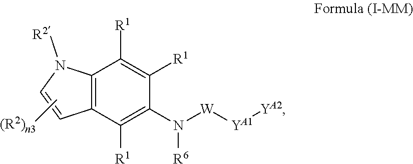

wherein: R^{2′} is H or R²; and n3 is 0 or 1.

In certain of these embodiments, n3=0. In certain other embodiments, n3=1. In certain embodiments, R^{2′} is H.

In certain embodiments of Formula (I-MM), each R¹ is H. In certain other embodiments, two R¹ are H; and the remaining R¹ is other than H. In certain of these embodiments, one R¹ is selected from the consisting of: halo, cyano, —C(═O)(C₁₋₄ alkyl), —C(═O)OH, and C₁₋₆ alkyl optionally substituted with 1-2 R^{a}.

In certain embodiments of Formula (I-MM), R² is independently selected from the group consisting of halo, cyano, —C(═O)O(C₁₋₄ alkyl), —C(═O)OH, and C₁₋₆ alkyl optionally substituted with 1-2 R^{a}.

In certain embodiments of Formula (I-MM), W is *C(═O)NR^{N}. In certain of these embodiments, W is *C(═O)NH.

In certain embodiments of Formula (I-MM) Y^{A1} is a bond.

In certain other embodiments, Y^{A1} is C₁₋₆ alkylene which is optionally substituted with from 1-2 R^{a}. For example, Y^{A1} can be —CH₂—, —CH₂CH₂—, —CH₂CH₂CH₂—, —CH(CF₃)—, —CH₂CH(OH)—,

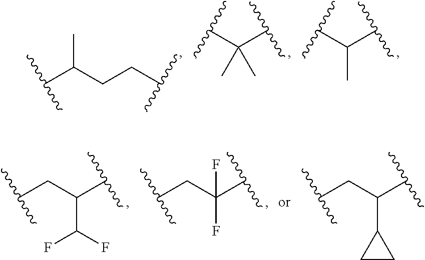

(e.g., Y^{A1} is CH₂).

In certain embodiments of Formula (I-MM), Y^{A2} is

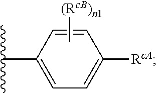

n1 is 0, 1, or 2; and each of R^{cA} and R^{cB} is an independently selected R^{c}.

In certain embodiments of Formula (I-MM), Y^{A2} is

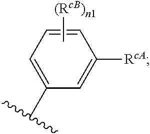

n1 is 0, 1, or 2; and each of R^{cA} and R^{cB} is an independently selected R^{c}.

In certain embodiments of Formula (I-MM), Y^{A2} is

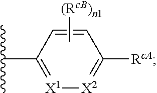

one of X¹ and X² is N; the other one of X¹ and X² is CH; n1 is 0, 1, or 2; and each of R^{cA} and R^{cB} is an independently selected R^{c}.

In certain embodiments of Formula (I-MM), Y^{2A} is

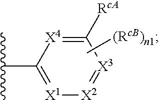

one of X¹, X², X³, and X⁴ is N; each of the remaining of X¹, X², X³, and X⁴ is CH; n1 is 0, 1, or 2; and each of R^{cA} and R^{cB} is an independently selected R^{c}.

In certain embodiments of Formula (I-MM), R^{cA} is C₁₋₃ alkyl which is substituted with from 1-3 —F (e.g., R^{cA} is —CF₃).

In certain embodiments of Formula (I-MM), R^{cA} is unsubstituted C₁₋₁₀ alkyl (e.g., straight chain C₂, C₃, C₄, C₅, C₆, or C₇₋₁₀ alkyl); or

In certain embodiments of Formula (I-MM), R^{cA} is C₂₋₆ alkenyl, C₂₋₆ alkynyl, or —C(═O)(C₁₋₁₀ alkyl) (e.g., —C(═O)(C₃₋₁₀ alkyl) (e.g., —C(═O)CH₂CH₂CH₂CH₂CH₂CH₂CH₂));

In certain embodiments of Formula (I-MM), R^{cA} is selected from the group consisting of —SF₅, —S(O)₁₋₂(NR′R″) (e.g.,

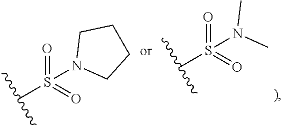

S(O)₁₋₂(C₁₋₄ alkyl), and S(O)₁₋₂(C₁₋₄ haloalkyl) (e.g., S(O)₂CF₃).

In certain embodiments of Formula (I-MM), R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

In certain embodiments of Formula (I-MM), R^{cA} is -L¹-L²-R^{h}.

In certain of these embodiments, -L¹ is a bond. In certain other embodiments, -L¹ is CH₂, or —CH₂CH₂. In certain embodiments, -L² is a bond or —O—.

In certain embodiments, R^{h} is C₃₋₆ cycloalkyl optionally substituted with from 1-4 substituents independently selected from the group consisting of halo, C₁₋₄ alkyl, and C₁₋₄ haloalkyl (e.g.,

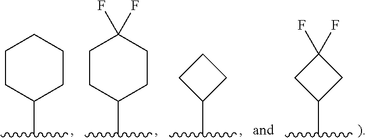

In certain embodiments, R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

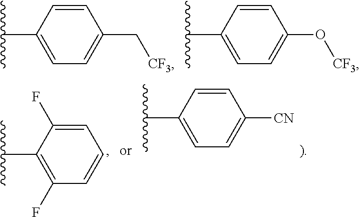

In certain embodiments of Formula (I-MM), n1 is 0. In certain other embodiments, n1 is 1. In certain of these embodiments, each R^{cB} is independently halo or C₁₋₄ alkyl optionally substituted with R^{a}.

In certain embodiments of Formula (I-MM), R⁶ is H.

The detailed description concludes with 383 numbered clauses, which further describe the compounds, compositions, methods, and other subject matter described herein. For ease of exposition, certain variable definitions refer to one or more specifically numbered clauses. For the avoidance of doubt, use of a phrase, such as “each occurrence of R^{b} is as defined in clause 189” is intended to mean that:

each occurrence of R^{b} substituent of Y^{A2} is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

Compound Provisions

In some embodiments, it is provided that when the compound has Formula (I-a1) wherein R^{2′} is H or R², W-A is defined according to (A), and W is *C(O)N^{N} (e.g., *C(O)NH—), then 1, 2, 3, 4, or 5 of the following provisions apply:

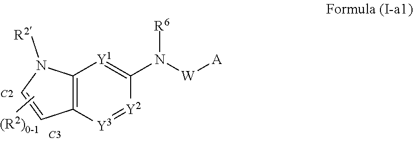

(i) when each of Y¹ and Y² is CH; Y³ is CR¹; R¹ is CO₂Me, CO₂Et, CN, or Cl (e.g., R^{2′} is H); and R² is absent (i.e., C2 and C3 are substituted with H), OR when each of Y¹ and Y² is N; and Y³ is OH or oxo, then A cannot be optionally substituted C₁₋₆ alkyl, such as methyl or butyl; 1,1,3,3-tetramethylbutyl; or optionally substituted C₃ or C₆ cycloalkyl (such as C₁₋₆ alkyl or C₃ or C₆ cycloalkyl optionally substituted with CO₂H, isocyanate, or substituted amino);

(ii) when each of Y¹ and Y² is N; and Y³ is CR¹; then
R¹ cannot be furyl, when W-A is benzyl; and
R¹ cannot be substituted N-linked aniline or chloro when either R^{2′} is methyl or when W-A is phenyl substituted with from 1-2 substituents independently selected from —Cl , —F, —Br, and CF₃;

(iii) when each of Y¹, Y², and; Y³ is CH; R^{2′} is H, R² is present and attached at the C3-position of the indole ring; and A is phenyl, tolyl, optionally substituted quinazolinyl, optionally substituted pyrazolyl, optionally substituted indolyl, optionally substituted naphthyl, or optionally substituted moropholinyl-phenyl, then R² cannot be oxazolyl, pyridyl, C-linked-2-pyridylethyl, phenyl, cyano, or C(O)NH₂;

(iv) when each of each of Y¹ and Y³ is CH; Y² is CH or CMe; R^{2′} is H; and R² is absent, then:
R^{h} cannot be a fused tricyclic ring;
Y^{A2} cannot be optionally substituted cyclohexyl, cyclohexenyl, imidazo[1,2-a][1,4]benzodiazepin-4-yl, indenyl, naphthyl, or tetrahydronaphthyl;
Y^{A1} cannot be alkylene substituted with phenyl;
when Y^{A1} is alkylene, Y^{A2} cannot be phenyl or the following substituted phenyl rings: 4-Br, 2,4-(Cl)₂, 3-propenyl, 2,3-(OMe)₂, and 4-CF₃; and
when Y^{A1} is absent, Y^{A2} cannot be phenyl or the following substituted phenyl rings: 3-NO₂, 4-Br, 2,4-(Cl)₂, 2,3-(OMe)₂, 4-CF₃, 4-CO₂Et, 3-CF₃-4-Cl, 2-Cl-4 CF₃, 2-OEt, 2-OMe-4-NO₂, 3,4-(OMe)₂, 2,4-(Me)₂, 3,4-(Cl )₂, 2,4-(F)₂, 2-Et, 2-F, 2-Me, 2-Br, 2-Cl-4-Br, 2-CF₃, 2,4-(OMe)₂, 2,3-(Me)₂, 3,5-(Cl )₂, 3-CF₃-4-F, 4-iso-propyl, 4-OMe, 4-Cl, 3-F-4-Me, 3-CF₃, 2,5-(OMe)₂, 2-Me-3-Cl, 2,3-(Me)₂, 2,3-(Cl )₂, 4-Bu, 3-OMe, 3-Cl, 4-Me-2-Cl, 3-SMe, 2-CO₂Me, 4-Me-3-Cl, 3,4-(Me)₂, 4-sec-butyl, 2-OMe, 2-Cl, 2,4-(OMe)₂-5-Cl, 4-OEt, 4-acetyl, 2-OMe-5-Me, 2-Me-5-Cl, 3,5-(Me)₂, 3,5-(Cl )₂, 4-NO₂, 4-Br, 4-F, 4-Me, 4-Et, 3-F, 3-Me, 3-acetyl, or 2-Me-5-Cl ; and

(v) the compound is other than:

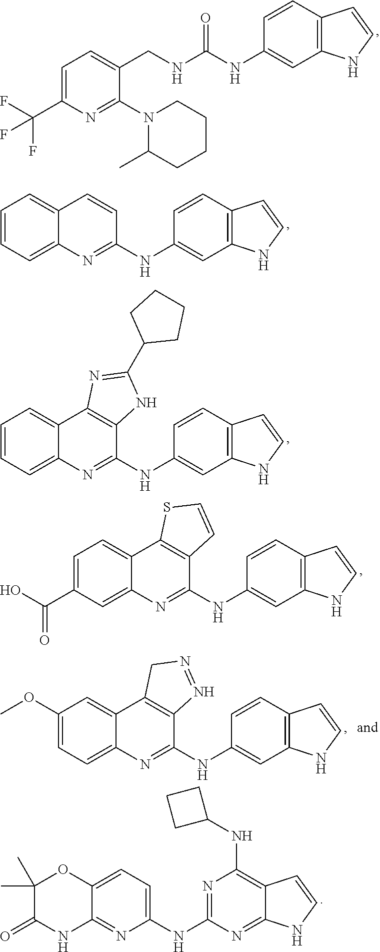

In some embodiments, it is provided that when the compound has Formula (I-a1) wherein R^{2′} is H or R², W-A is defined according to (A), and W is —*C(O)NH—:

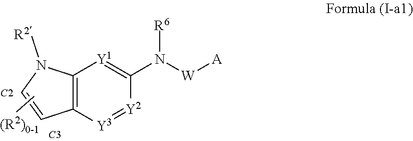

then Y³ is CR¹ which is other than CH. For example, Y³ can be C-cyano or C-halo (e.g., C-Cl or C-F).

Non-Limiting Exemplary Compounds

In certain embodiments, the compound is selected from the group consisting of the compounds delineated in Table C1 (infra) or a pharmaceutically acceptable salt thereof:

TABLE C1

| Compound | |
|---|---|
| No. | Structure |
| 101 | 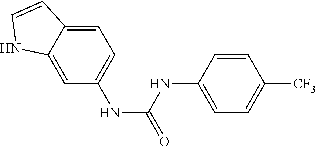 [SMILES: O=C(Nc1ccc(C(F)(F)F)cc1)Nc1ccc2cc[nH]c2c1] |
| 102 | 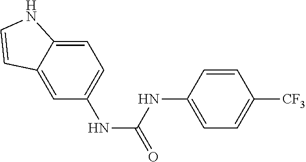 [SMILES: O=C(Nc1ccc(C(F)(F)F)cc1)Nc1ccc2[nH]ccc2c1] |
| 103 | 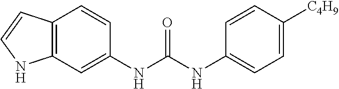 |
| 104 | 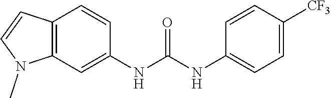 [SMILES: Cn1ccc2ccc(NC(=O)Nc3ccc(C(F)(F)F)cc3)cc21] |
| 105 | 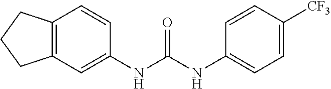 [SMILES: O=C(Nc1ccc(C(F)(F)F)cc1)Nc1ccc2c(c1)CCC2] |
| 106 | 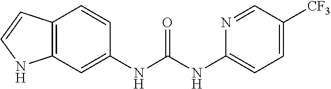 [SMILES: O=C(Nc1ccc2cc[nH]c2c1)Nc1ccc(C(F)(F)F)cn1] |
| 107 | 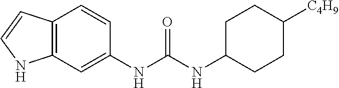 |
| 108 | 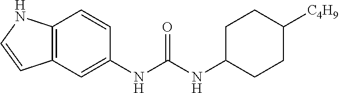 |
| 109 | 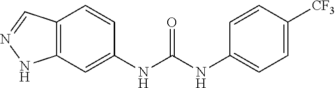 |
| 110 | 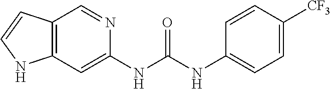 [SMILES: O=C(Nc1ccc(C(F)(F)F)cc1)Nc1cc2[nH]ccc2cn1] |
| 111 | 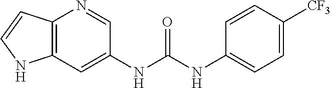 [SMILES: O=C(Nc1ccc(C(F)(F)F)cc1)Nc1cnc2cc[nH]c2c1] |
| 112 | 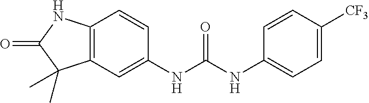 [SMILES: CC1(C)C(=O)Nc2ccc(NC(=O)Nc3ccc(C(F)(F)F)cc3)cc21] |
| 113 | 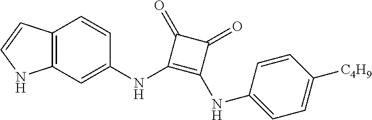 |
| 114 | 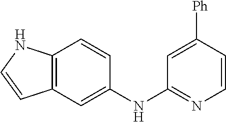 [SMILES: c1ccc(-c2ccnc(Nc3ccc4[nH]ccc4c3)c2)cc1] |
| 115 | 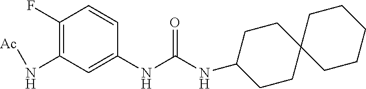 [SMILES: CC(=O)Nc1cc(NC(=O)NC2CCC3(CCCCC3)CC2)ccc1F] |
| 116 | 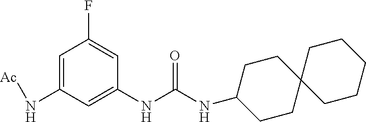 [SMILES: CC(=O)Nc1cc(F)cc(NC(=O)NC2CCC3(CCCCC3)CC2)c1] |
| 117 | 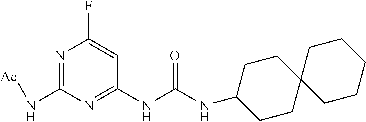 [SMILES: CC(=O)Nc1nc(F)cc(NC(=O)NC2CCC3(CCCCC3)CC2)n1] |
| 118 | 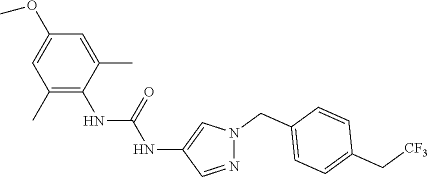 [SMILES: COc1cc(C)c(NC(=O)Nc2cnn(Cc3ccc(CC(F)(F)F)cc3)c2)c(C)c1] |
| 119 | 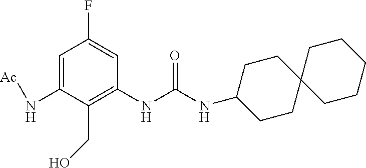 [SMILES: "Ac" ; Nc1cc(F)cc(NC(=O)NC2CCC3(CCCCC3)CC2)c1CO] |
| 120 | 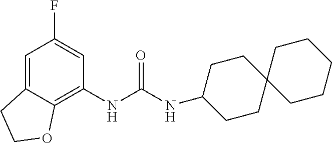 [SMILES: O=C(Nc1cc(F)cc2c1OCC2)NC1CCC2(CCCCC2)CC1] |
| 121 | 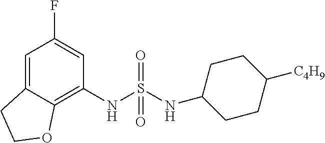 |
| 122 | 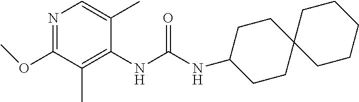 [SMILES: COc1ncc(C)c(NC(=O)NC2CCC3(CCCCC3)CC2)c1C] |
| 123 | 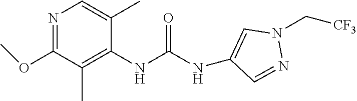 [SMILES: COc1ncc(C)c(NC(=O)Nc2cnn(CC(F)(F)F)c2)c1C] |
| 124 | 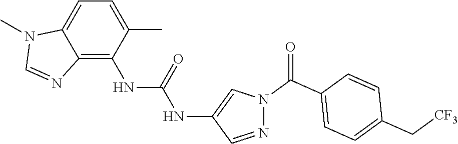 [SMILES: Cc1ccc2c(ncn2C)c1NC(=O)Nc1cnn(C(=O)c2ccc(CC(F)(F)F)cc2)c1] |
| 125 | 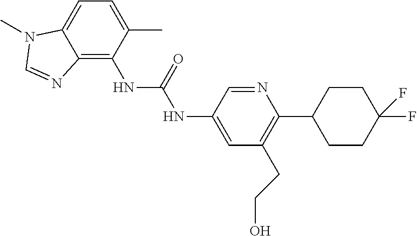 [SMILES: Cc1ccc2c(ncn2C)c1NC(=O)Nc1cnc(C2CCC(F)(F)CC2)c(CCO)c1] |
| 126 | 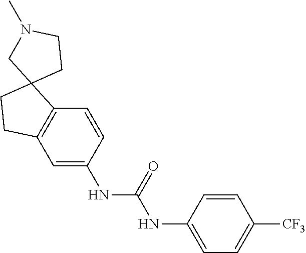 [SMILES: CN1CCC2(CCc3cc(NC(=O)Nc4ccc(C(F)(F)F)cc4)ccc32)C1] |
| 127 | 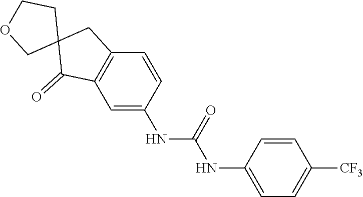 [SMILES: O=C(Nc1ccc(C(F)(F)F)cc1)Nc1ccc2c(c1)C(=O)C1(CCOC1)C2] |
| 128 | 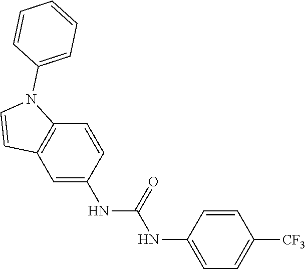 [SMILES: O=C(Nc1ccc(C(F)(F)F)cc1)Nc1ccc2c(ccn2-c2ccccc2)c1] |
| 129 | 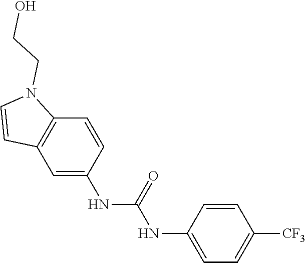 [SMILES: O=C(Nc1ccc(C(F)(F)F)cc1)Nc1ccc2c(ccn2CCO)c1] |
| 130 | 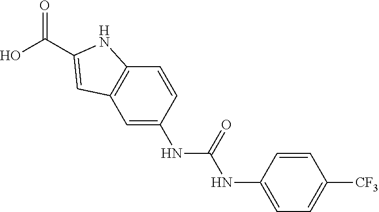 [SMILES: O=C(Nc1ccc(C(F)(F)F)cc1)Nc1ccc2[nH]c(C(=O)O)cc2c1] |
| 131 | 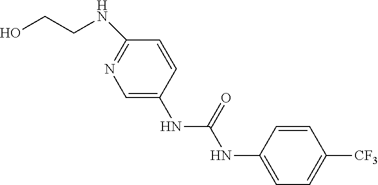 [SMILES: O=C(Nc1ccc(C(F)(F)F)cc1)Nc1ccc(NCCO)nc1] |
| 132 | 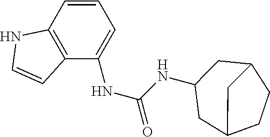 [SMILES: O=C(Nc1cccc2[nH]ccc12)NC1CC2CCC(C2)C1] |
| 133 | 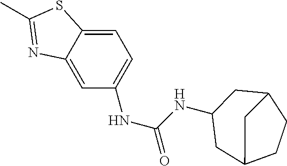 [SMILES: Cc1nc2cc(NC(=O)NC3CC4CCC(C4)C3)ccc2s1] |
| 134 | 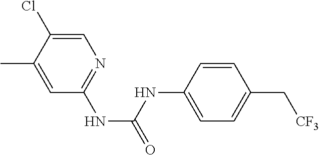 [SMILES: Cc1cc(NC(=O)Nc2ccc(CC(F)(F)F)cc2)ncc1Cl] |
| 135 | 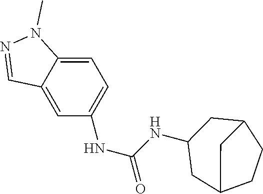 [SMILES: Cn1ncc2cc(NC(=O)NC3CC4CCC(C4)C3)ccc21] |
| 136 | 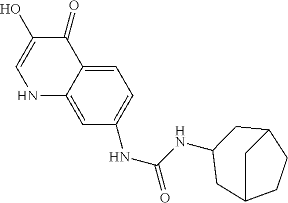 [SMILES: O=C(Nc1ccc2c(=O)c(O)c[nH]c2c1)NC1CC2CCC(C2)C1] |
| 137 | 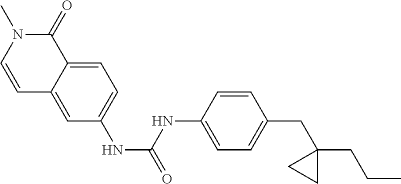 [SMILES: CCCC1(Cc2ccc(NC(=O)Nc3ccc4c(=O)n(C)ccc4c3)cc2)CC1] |
| 138 | 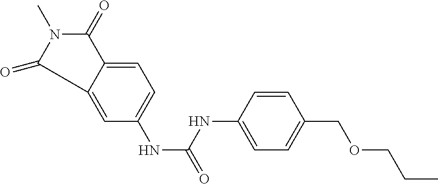 [SMILES: CCCOCc1ccc(NC(=O)Nc2ccc3c(c2)C(=O)N(C)C3=O)cc1] |
| 139 | 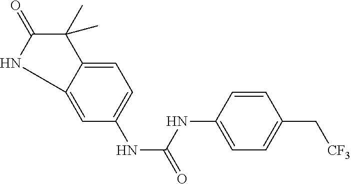 [SMILES: CC1(C)C(=O)Nc2cc(NC(=O)Nc3ccc(CC(F)(F)F)cc3)ccc21] |
| 140 | 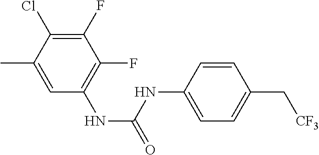 [SMILES: Cc1cc(NC(=O)Nc2ccc(CC(F)(F)F)cc2)c(F)c(F)c1Cl] |
| 141 | 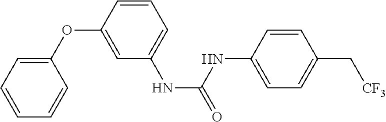 [SMILES: O=C(Nc1ccc(CC(F)(F)F)cc1)Nc1cccc(Oc2ccccc2)c1] |
| 142 | 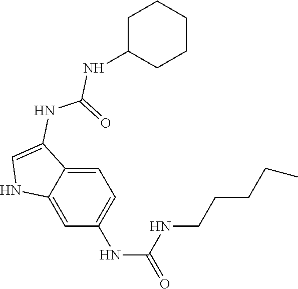 [SMILES: CCCCCNC(=O)Nc1ccc2c(NC(=O)NC3CCCCC3)c[nH]c2c1] |
| 143 | 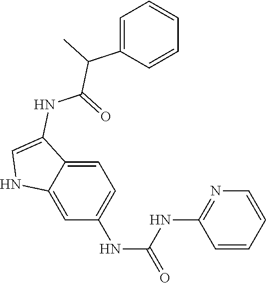 [SMILES: CC(C(=O)Nc1c[nH]c2cc(NC(=O)Nc3ccccn3)ccc12)c1ccccc1] |
| 144 | 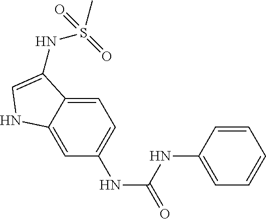 [SMILES: CS(=O)(=O)Nc1c[nH]c2cc(NC(=O)Nc3ccccc3)ccc12] |
| 145 | 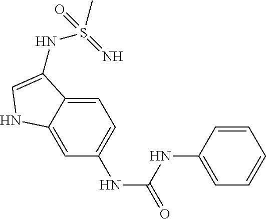 [SMILES: CS(=N)(=O)Nc1c[nH]c2cc(NC(=O)Nc3ccccc3)ccc12] |
| 146 | 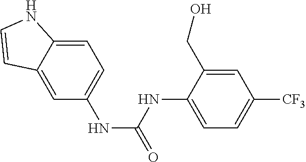 [SMILES: O=C(Nc1ccc2[nH]ccc2c1)Nc1ccc(C(F)(F)F)cc1CO] |
| 147 | 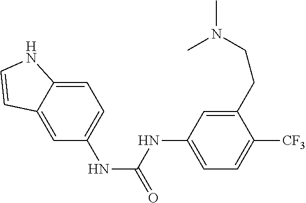 [SMILES: CN(C)CCc1cc(NC(=O)Nc2ccc3[nH]ccc3c2)ccc1C(F)(F)F] |
| 148 | 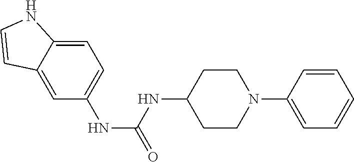 [SMILES: O=C(Nc1ccc2[nH]ccc2c1)NC1CCN(c2ccccc2)CC1] |
| 149 | 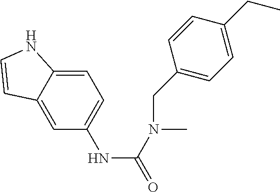 [SMILES: CCc1ccc(CN(C)C(=O)Nc2ccc3[nH]ccc3c2)cc1] |
| 150 | 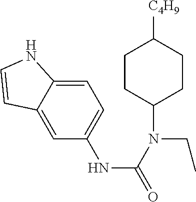 |
| 151 | 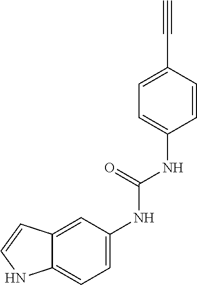 [SMILES: C#Cc1ccc(NC(=O)Nc2ccc3[nH]ccc3c2)cc1] |
| 152 | 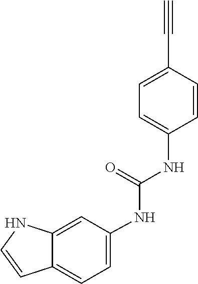 [SMILES: C#Cc1ccc(NC(=O)Nc2ccc3cc[nH]c3c2)cc1] |
| 152a | 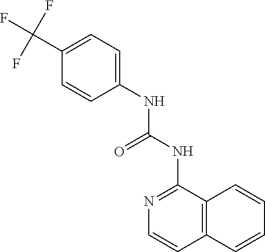 [SMILES: O=C(Nc1ccc(C(F)(F)F)cc1)Nc1nccc2ccccc12] |
| 152b | 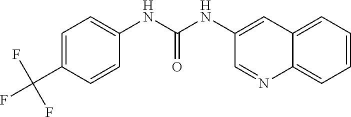 [SMILES: O=C(Nc1ccc(C(F)(F)F)cc1)Nc1cnc2ccccc2c1] |
| 153 | 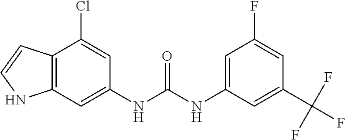 [SMILES: O=C(Nc1cc(F)cc(C(F)(F)F)c1)Nc1cc(Cl)c2cc[nH]c2c1] |
| 154 | 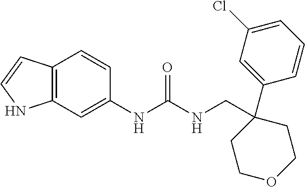 [SMILES: O=C(NCC1(c2cccc(Cl)c2)CCOCC1)Nc1ccc2cc[nH]c2c1] |
| 155 | 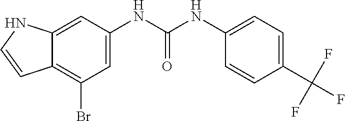 [SMILES: O=C(Nc1ccc(C(F)(F)F)cc1)Nc1cc(Br)c2cc[nH]c2c1] |
| 156 | 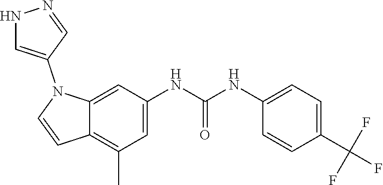 [SMILES: Cc1cc(NC(=O)Nc2ccc(C(F)(F)F)cc2)cc2c1ccn2-c1cn[nH]c1] |
| 157 | 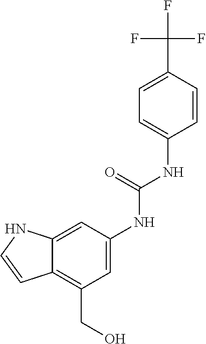 [SMILES: O=C(Nc1ccc(C(F)(F)F)cc1)Nc1cc(CO)c2cc[nH]c2c1] |
| 158 | 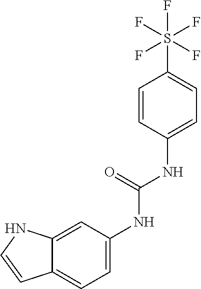 [SMILES: O=C(Nc1ccc(S(F)(F)(F)(F)F)cc1)Nc1ccc2cc[nH]c2c1] |
| 159 | 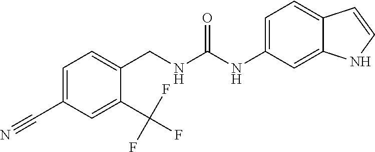 [SMILES: N#Cc1ccc(CNC(=O)Nc2ccc3cc[nH]c3c2)c(C(F)(F)F)c1] |
| 160 | 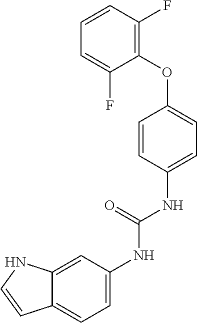 [SMILES: O=C(Nc1ccc(Oc2c(F)cccc2F)cc1)Nc1ccc2cc[nH]c2c1] |
| 161 | 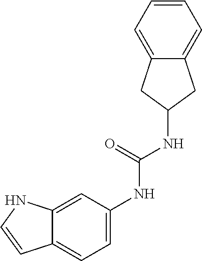 [SMILES: O=C(Nc1ccc2cc[nH]c2c1)NC1Cc2ccccc2C1] |
| 162 | 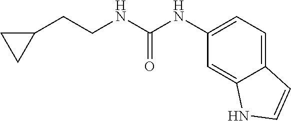 [SMILES: O=C(NCCC1CC1)Nc1ccc2cc[nH]c2c1] |
| 163 | 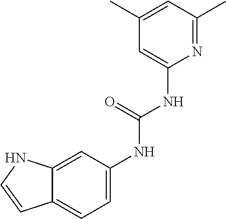 [SMILES: Cc1cc(C)nc(NC(=O)Nc2ccc3cc[nH]c3c2)c1] |
| 164 | 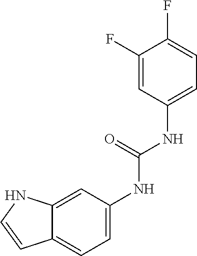 [SMILES: O=C(Nc1ccc(F)c(F)c1)Nc1ccc2cc[nH]c2c1] |
| 165 | 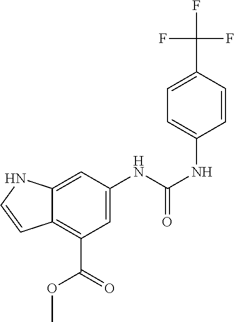 [SMILES: COC(=O)c1cc(NC(=O)Nc2ccc(C(F)(F)F)cc2)cc2[nH]ccc12] |
| 166 | 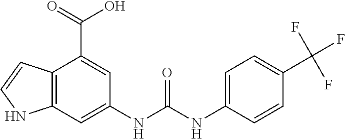 [SMILES: O=C(Nc1ccc(C(F)(F)F)cc1)Nc1cc(C(=O)O)c2cc[nH]c2c1] |
| 167 | 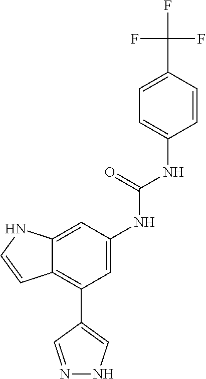 [SMILES: O=C(Nc1ccc(C(F)(F)F)cc1)Nc1cc(-c2cn[nH]c2)c2cc[nH]c2c1] |
| 168 | 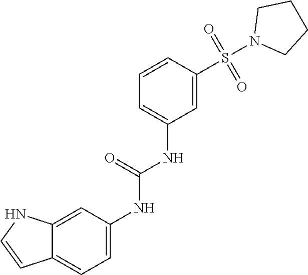 [SMILES: O=C(Nc1cccc(S(=O)(=O)N2CCCC2)c1)Nc1ccc2cc[nH]c2c1] |
| 169 | 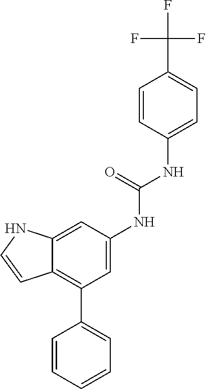 [SMILES: O=C(Nc1ccc(C(F)(F)F)cc1)Nc1cc(-c2ccccc2)c2cc[nH]c2c1] |
| 170 | 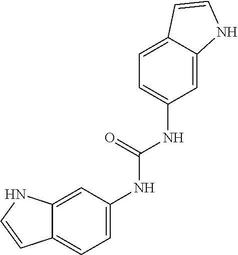 [SMILES: O=C(Nc1ccc2cc[nH]c2c1)Nc1ccc2cc[nH]c2c1] |
| 171 | 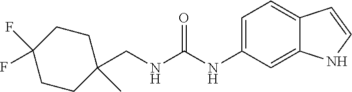 [SMILES: CC1(CNC(=O)Nc2ccc3cc[nH]c3c2)CCC(F)(F)CC1] |
| 172 | 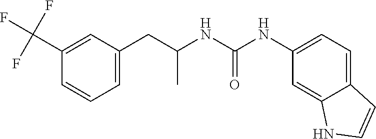 [SMILES: CC(Cc1cccc(C(F)(F)F)c1)NC(=O)Nc1ccc2cc[nH]c2c1] |
| 173 | 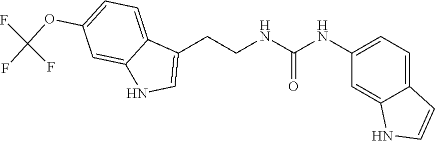 [SMILES: O=C(NCCc1c[nH]c2cc(OC(F)(F)F)ccc12)Nc1ccc2cc[nH]c2c1] |
| 174 | 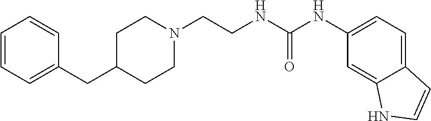 [SMILES: O=C(NCCN1CCC(Cc2ccccc2)CC1)Nc1ccc2cc[nH]c2c1] |
| 175 | 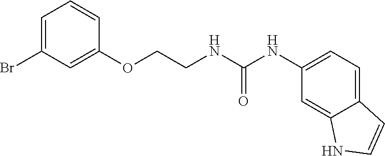 [SMILES: O=C(NCCOc1cccc(Br)c1)Nc1ccc2cc[nH]c2c1] |
| 176 | 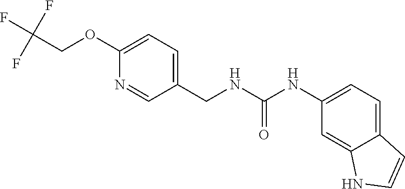 [SMILES: O=C(NCc1ccc(OCC(F)(F)F)nc1)Nc1ccc2cc[nH]c2c1] |
| 177 | 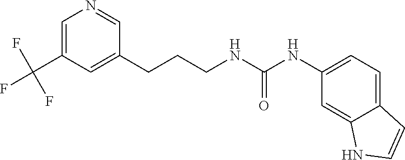 [SMILES: O=C(NCCCc1cncc(C(F)(F)F)c1)Nc1ccc2cc[nH]c2c1] |
| 178 | 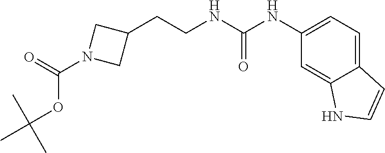 [SMILES: CC(C)(C)OC(=O)N1CC(CCNC(=O)Nc2ccc3cc[nH]c3c2)C1] |
| 179 | 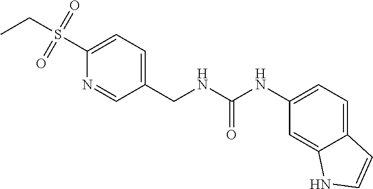 [SMILES: CCS(=O)(=O)c1ccc(CNC(=O)Nc2ccc3cc[nH]c3c2)cn1] |
| 180 | 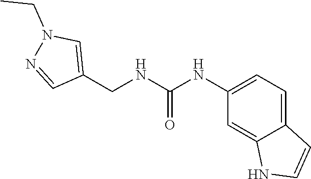 [SMILES: CCn1cc(CNC(=O)Nc2ccc3cc[nH]c3c2)cn1] |
| 181 | 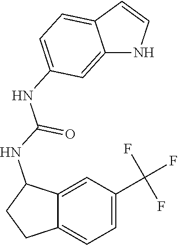 [SMILES: O=C(Nc1ccc2cc[nH]c2c1)NC1CCc2ccc(C(F)(F)F)cc21] |
| 182 | 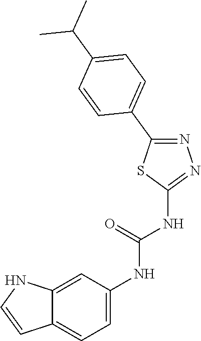 [SMILES: CC(C)c1ccc(-c2nnc(NC(=O)Nc3ccc4cc[nH]c4c3)s2)cc1] |
| 183 | 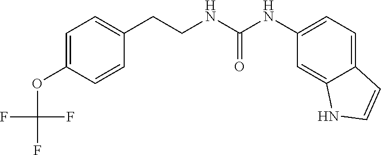 [SMILES: O=C(NCCc1ccc(OC(F)(F)F)cc1)Nc1ccc2cc[nH]c2c1] |
| 184 | 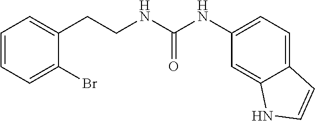 [SMILES: O=C(NCCc1ccccc1Br)Nc1ccc2cc[nH]c2c1] |
| 185 | 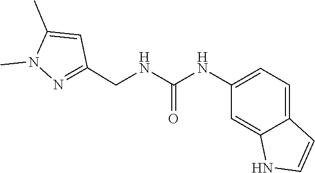 [SMILES: Cc1cc(CNC(=O)Nc2ccc3cc[nH]c3c2)nn1C] |
| 186 | 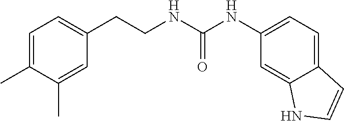 [SMILES: Cc1ccc(CCNC(=O)Nc2ccc3cc[nH]c3c2)cc1C] |
| 187 | 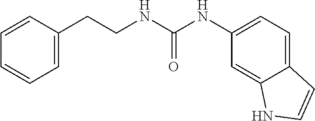 [SMILES: O=C(NCCc1ccccc1)Nc1ccc2cc[nH]c2c1] |
| 188 | 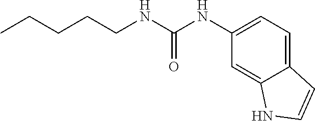 [SMILES: CCCCCNC(=O)Nc1ccc2cc[nH]c2c1] |
| 189 | 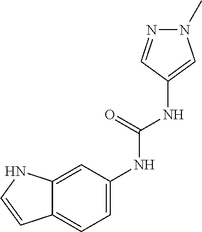 [SMILES: Cn1cc(NC(=O)Nc2ccc3cc[nH]c3c2)cn1] |
| 190 | 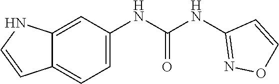 [SMILES: O=C(Nc1ccc2cc[nH]c2c1)Nc1ccon1] |
| 191 | 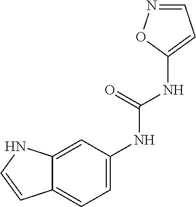 [SMILES: O=C(Nc1ccc2cc[nH]c2c1)Nc1ccno1] |
| 192 | 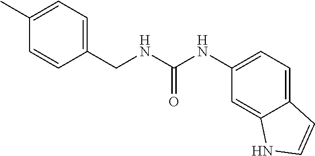 [SMILES: Cc1ccc(CNC(=O)Nc2ccc3cc[nH]c3c2)cc1] |
| 193 | 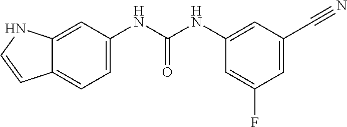 [SMILES: N#Cc1cc(F)cc(NC(=O)Nc2ccc3cc[nH]c3c2)c1] |
| 194 | 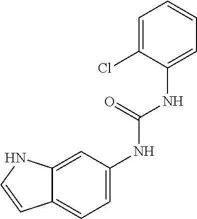 [SMILES: O=C(Nc1ccc2cc[nH]c2c1)Nc1ccccc1Cl] |
| 195 | 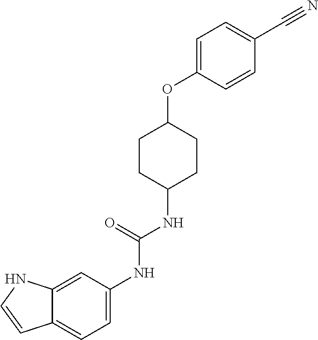 [SMILES: N#Cc1ccc(OC2CCC(NC(=O)Nc3ccc4cc[nH]c4c3)CC2)cc1] |
| 196 | 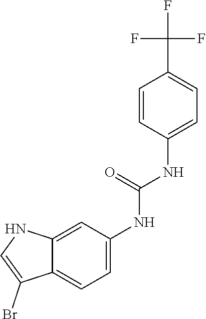 [SMILES: O=C(Nc1ccc(C(F)(F)F)cc1)Nc1ccc2c(Br)c[nH]c2c1] |
| 197 | 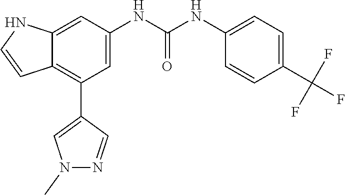 [SMILES: Cn1cc(-c2cc(NC(=O)Nc3ccc(C(F)(F)F)cc3)cc3[nH]ccc23)cn1] |
| 198 | 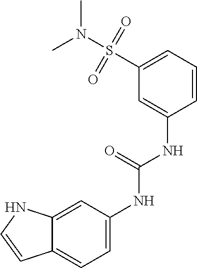 [SMILES: CN(C)S(=O)(=O)c1cccc(NC(=O)Nc2ccc3cc[nH]c3c2)c1] |
| 199 | 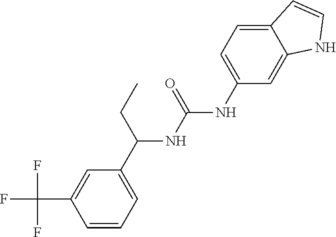 [SMILES: CCC(NC(=O)Nc1ccc2cc[nH]c2c1)c1cccc(C(F)(F)F)c1] |
| 200 | 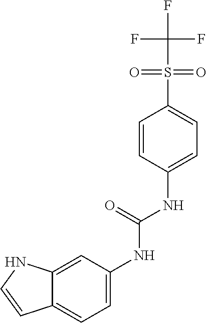 [SMILES: O=C(Nc1ccc(S(=O)(=O)C(F)(F)F)cc1)Nc1ccc2cc[nH]c2c1] |
| 201 | 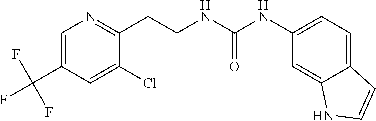 [SMILES: O=C(NCCc1ncc(C(F)(F)F)cc1Cl)Nc1ccc2cc[nH]c2c1] |
| 202 | 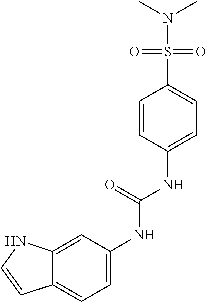 [SMILES: CN(C)S(=O)(=O)c1ccc(NC(=O)Nc2ccc3cc[nH]c3c2)cc1] |
| 203 | 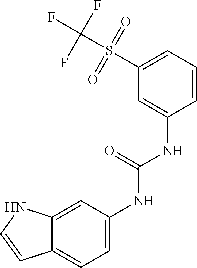 [SMILES: O=C(Nc1cccc(S(=O)(=O)C(F)(F)F)c1)Nc1ccc2cc[nH]c2c1] |
| 204 | 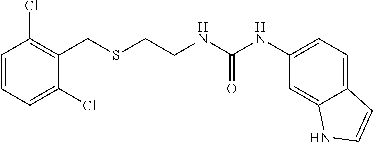 [SMILES: O=C(NCCSCc1c(Cl)cccc1Cl)Nc1ccc2cc[nH]c2c1] |
| 205 | 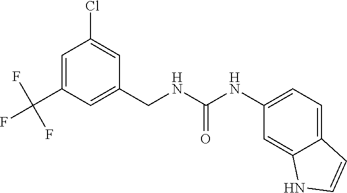 [SMILES: O=C(NCc1cc(Cl)cc(C(F)(F)F)c1)Nc1ccc2cc[nH]c2c1] |
| 206 | 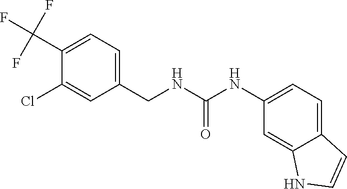 [SMILES: O=C(NCc1ccc(C(F)(F)F)c(Cl)c1)Nc1ccc2cc[nH]c2c1] |
| 207 | 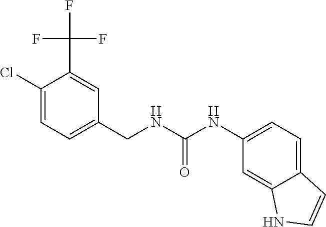 [SMILES: O=C(NCc1ccc(Cl)c(C(F)(F)F)c1)Nc1ccc2cc[nH]c2c1] |
| 208 | 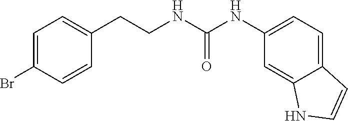 [SMILES: O=C(NCCc1ccc(Br)cc1)Nc1ccc2cc[nH]c2c1] |
| 209 | 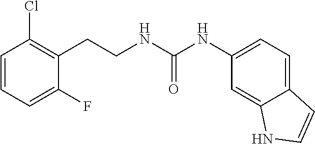 [SMILES: O=C(NCCc1c(F)cccc1Cl)Nc1ccc2cc[nH]c2c1] |
| 210 | 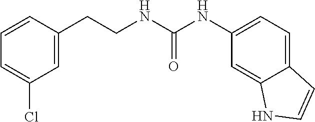 [SMILES: O=C(NCCc1cccc(Cl)c1)Nc1ccc2cc[nH]c2c1] |
| 211 | 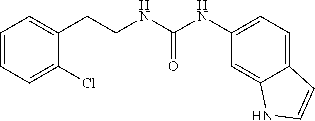 [SMILES: O=C(NCCc1ccccc1Cl)Nc1ccc2cc[nH]c2c1] |
| 212 | 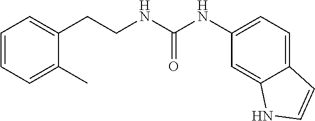 [SMILES: Cc1ccccc1CCNC(=O)Nc1ccc2cc[nH]c2c1] |
| 213 | 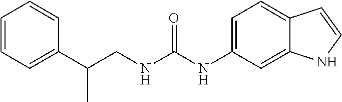 [SMILES: CC(CNC(=O)Nc1ccc2cc[nH]c2c1)c1ccccc1] |
| 214 | 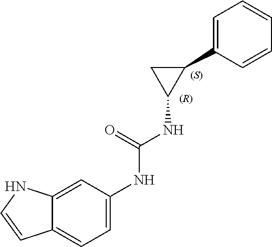 |
| 215 | 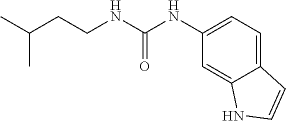 [SMILES: CC(C)CCNC(=O)Nc1ccc2cc[nH]c2c1] |
| 216 | 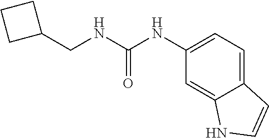 [SMILES: O=C(NCC1CCC1)Nc1ccc2cc[nH]c2c1] |
| 217 | 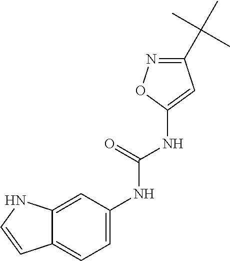 [SMILES: CC(C)(C)c1cc(NC(=O)Nc2ccc3cc[nH]c3c2)on1] |
| 218 | 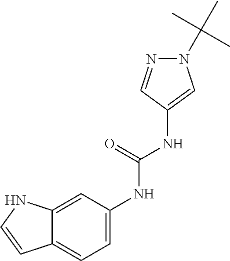 [SMILES: CC(C)(C)n1cc(NC(=O)Nc2ccc3cc[nH]c3c2)cn1] |
| 219 | 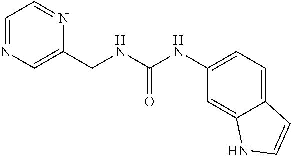 [SMILES: O=C(NCc1cnccn1)Nc1ccc2cc[nH]c2c1] |
| 220 | 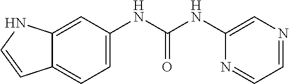 [SMILES: O=C(Nc1ccc2cc[nH]c2c1)Nc1cnccn1] |
| 221 | 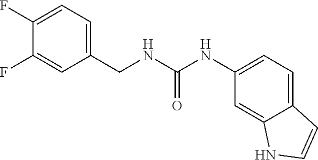 [SMILES: O=C(NCc1ccc(F)c(F)c1)Nc1ccc2cc[nH]c2c1] |
| 222 | 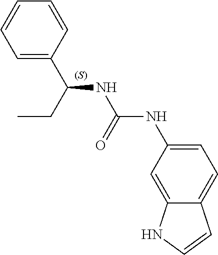 |
| 223 | 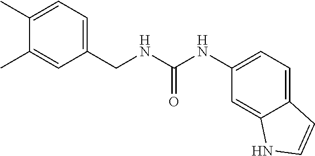 [SMILES: Cc1ccc(CNC(=O)Nc2ccc3cc[nH]c3c2)cc1C] |
| 224 | 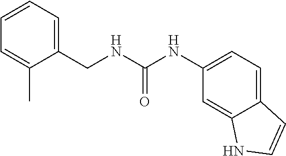 [SMILES: Cc1ccccc1CNC(=O)Nc1ccc2cc[nH]c2c1] |
| 225 | 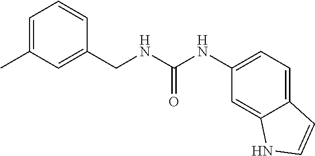 [SMILES: Cc1cccc(CNC(=O)Nc2ccc3cc[nH]c3c2)c1] |
| 226 | 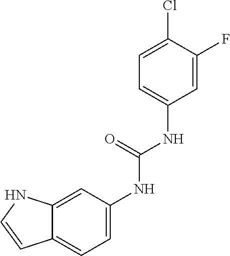 [SMILES: O=C(Nc1ccc(Cl)c(F)c1)Nc1ccc2cc[nH]c2c1] |
| 227 | 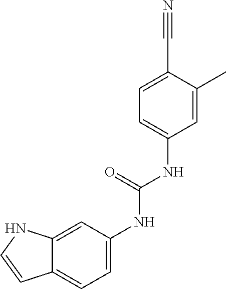 [SMILES: Cc1cc(NC(=O)Nc2ccc3cc[nH]c3c2)ccc1C#N] |
| 228 | 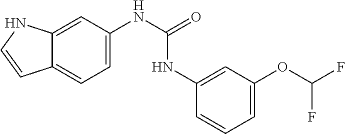 [SMILES: O=C(Nc1cccc(OC(F)F)c1)Nc1ccc2cc[nH]c2c1] |
| 229 | 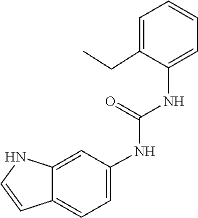 [SMILES: CCc1ccccc1NC(=O)Nc1ccc2cc[nH]c2c1] |
| 230 | 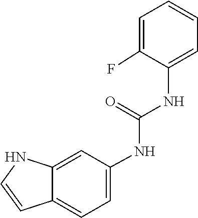 [SMILES: O=C(Nc1ccc2cc[nH]c2c1)Nc1ccccc1F] |
| 231 | 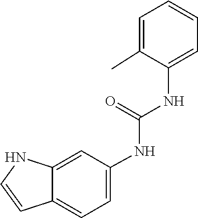 [SMILES: Cc1ccccc1NC(=O)Nc1ccc2cc[nH]c2c1] |
| 233 | 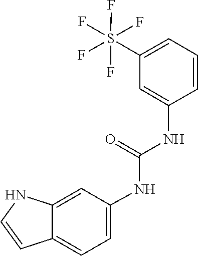 [SMILES: O=C(Nc1cccc(S(F)(F)(F)(F)F)c1)Nc1ccc2cc[nH]c2c1] |
| 234 | 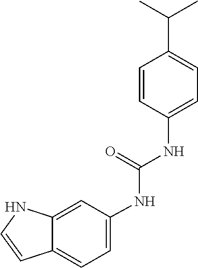 [SMILES: CC(C)c1ccc(NC(=O)Nc2ccc3cc[nH]c3c2)cc1] |
| 235 | 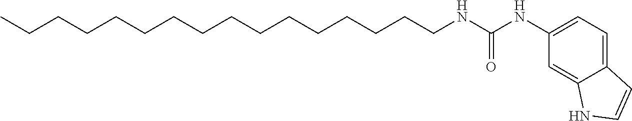 [SMILES: CCCCCCCCCCCCCCCCNC(=O)Nc1ccc2cc[nH]c2c1] |
| 237 | 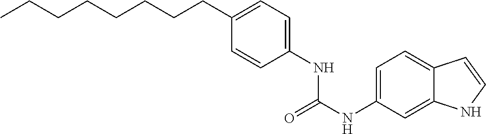 [SMILES: CCCCCCCCc1ccc(NC(=O)Nc2ccc3cc[nH]c3c2)cc1] |
| 236 | 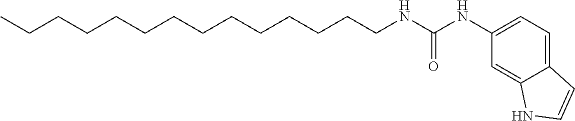 [SMILES: CCCCCCCCCCCCCCNC(=O)Nc1ccc2cc[nH]c2c1] |
| 240 | 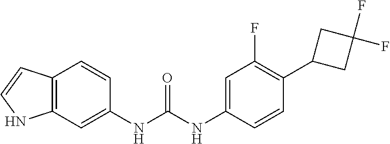 [SMILES: O=C(Nc1ccc(C2CC(F)(F)C2)c(F)c1)Nc1ccc2cc[nH]c2c1] |
| 242 | 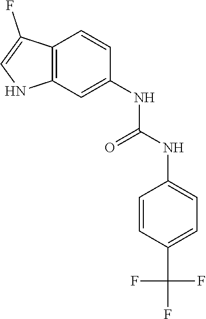 [SMILES: O=C(Nc1ccc(C(F)(F)F)cc1)Nc1ccc2c(F)c[nH]c2c1] |
| 243 | 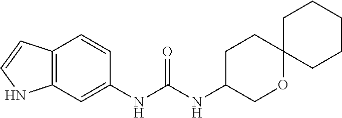 [SMILES: O=C(Nc1ccc2cc[nH]c2c1)NC1CCC2(CCCCC2)OC1] |
| 244 | 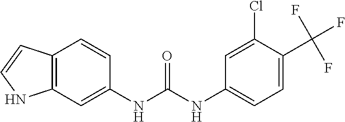 [SMILES: O=C(Nc1ccc(C(F)(F)F)c(Cl)c1)Nc1ccc2cc[nH]c2c1] |
| 245 | 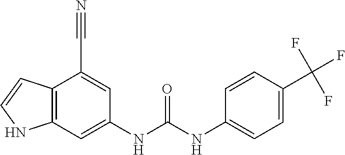 [SMILES: N#Cc1cc(NC(=O)Nc2ccc(C(F)(F)F)cc2)cc2[nH]ccc12] |
| 246 | 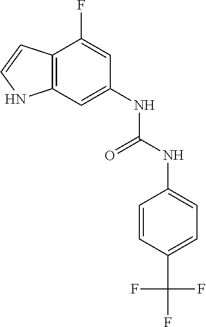 [SMILES: O=C(Nc1ccc(C(F)(F)F)cc1)Nc1cc(F)c2cc[nH]c2c1] |
| 247 | 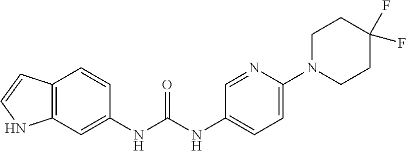 [SMILES: O=C(Nc1ccc(N2CCC(F)(F)CC2)nc1)Nc1ccc2cc[nH]c2c1] |
| 248 | 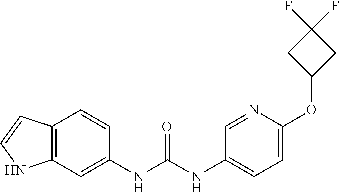 [SMILES: O=C(Nc1ccc(OC2CC(F)(F)C2)nc1)Nc1ccc2cc[nH]c2c1] |
| 249 | 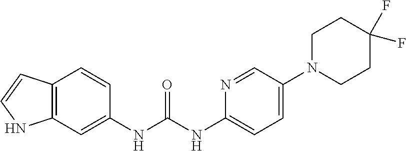 [SMILES: O=C(Nc1ccc2cc[nH]c2c1)Nc1ccc(N2CCC(F)(F)CC2)cn1] |
| 250 | 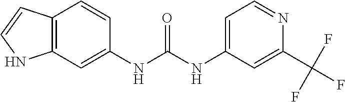 [SMILES: O=C(Nc1ccnc(C(F)(F)F)c1)Nc1ccc2cc[nH]c2c1] |
| 251 | 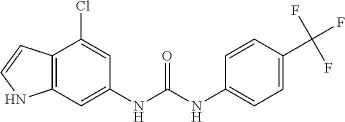 [SMILES: O=C(Nc1ccc(C(F)(F)F)cc1)Nc1cc(Cl)c2cc[nH]c2c1] |
| 252 | 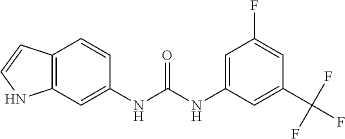 [SMILES: O=C(Nc1cc(F)cc(C(F)(F)F)c1)Nc1ccc2cc[nH]c2c1] |
| 253 | 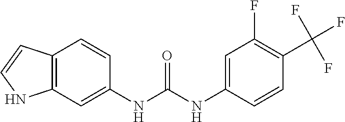 [SMILES: O=C(Nc1ccc(C(F)(F)F)c(F)c1)Nc1ccc2cc[nH]c2c1] |
| 254 | 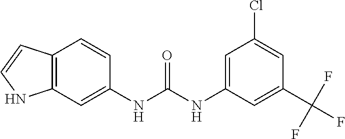 [SMILES: O=C(Nc1cc(Cl)cc(C(F)(F)F)c1)Nc1ccc2cc[nH]c2c1] |
| 255 | 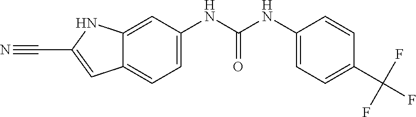 [SMILES: N#Cc1cc2ccc(NC(=O)Nc3ccc(C(F)(F)F)cc3)cc2[nH]1] |
| 256 | 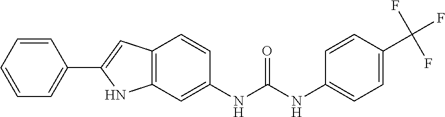 [SMILES: O=C(Nc1ccc(C(F)(F)F)cc1)Nc1ccc2cc(-c3ccccc3)[nH]c2c1] |
| 257 | 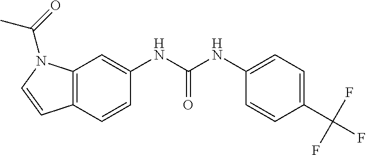 [SMILES: CC(=O)n1ccc2ccc(NC(=O)Nc3ccc(C(F)(F)F)cc3)cc21] |
| 258 | 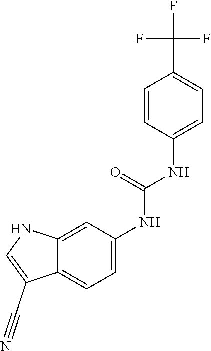 [SMILES: N#Cc1c[nH]c2cc(NC(=O)Nc3ccc(C(F)(F)F)cc3)ccc12] |
| 259 | 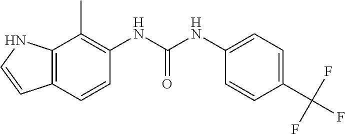 [SMILES: Cc1c(NC(=O)Nc2ccc(C(F)(F)F)cc2)ccc2cc[nH]c12] |
| 260 | 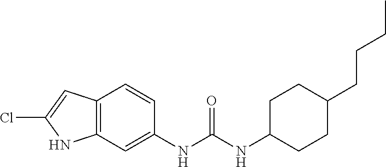 [SMILES: CCCCC1CCC(NC(=O)Nc2ccc3cc(Cl)[nH]c3c2)CC1] |
| 261 | 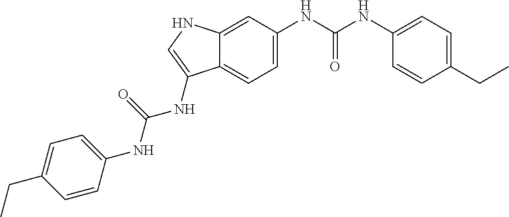 [SMILES: CCc1ccc(NC(=O)Nc2ccc3c(NC(=O)Nc4ccc(CC)cc4)c[nH]c3c2)cc1] |
| 262 | 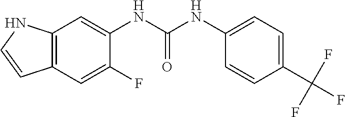 [SMILES: O=C(Nc1ccc(C(F)(F)F)cc1)Nc1cc2[nH]ccc2cc1F] |
| 263 | 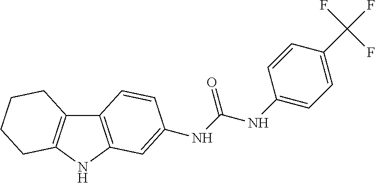 [SMILES: O=C(Nc1ccc(C(F)(F)F)cc1)Nc1ccc2c3c([nH]c2c1)CCCC3] |
| 264 | 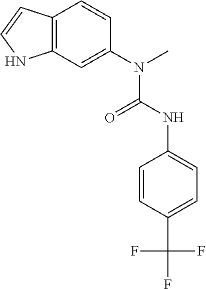 [SMILES: CN(C(=O)Nc1ccc(C(F)(F)F)cc1)c1ccc2cc[nH]c2c1] |
| 265 | 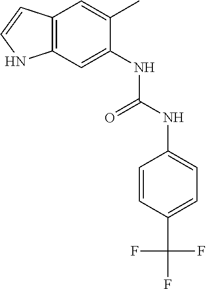 [SMILES: Cc1cc2cc[nH]c2cc1NC(=O)Nc1ccc(C(F)(F)F)cc1] |
| 266 | 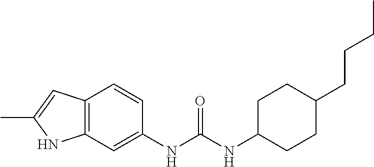 [SMILES: CCCCC1CCC(NC(=O)Nc2ccc3cc(C)[nH]c3c2)CC1] |
| 267 | 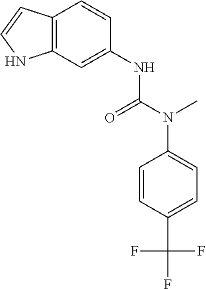 [SMILES: CN(C(=O)Nc1ccc2cc[nH]c2c1)c1ccc(C(F)(F)F)cc1] |
| 268 | 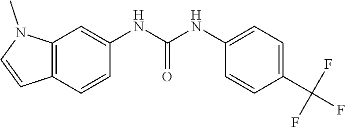 [SMILES: Cn1ccc2ccc(NC(=O)Nc3ccc(C(F)(F)F)cc3)cc21] |
| 269 | 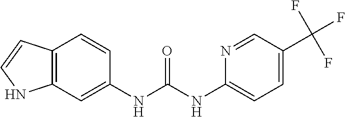 [SMILES: O=C(Nc1ccc2cc[nH]c2c1)Nc1ccc(C(F)(F)F)cn1] |
| 270 | 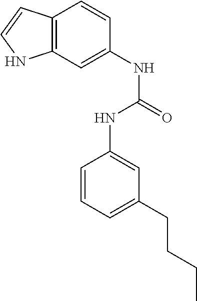 [SMILES: CCCCc1cccc(NC(=O)Nc2ccc3cc[nH]c3c2)c1] |
| 271 | 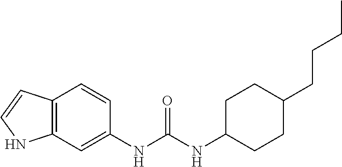 [SMILES: CCCCC1CCC(NC(=O)Nc2ccc3cc[nH]c3c2)CC1] |
| 272 | 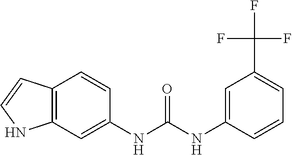 [SMILES: O=C(Nc1cccc(C(F)(F)F)c1)Nc1ccc2cc[nH]c2c1] |
| 273 | 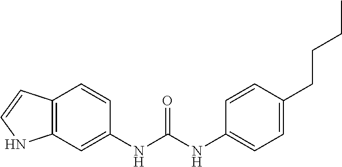 [SMILES: CCCCc1ccc(NC(=O)Nc2ccc3cc[nH]c3c2)cc1] |
| 274 | 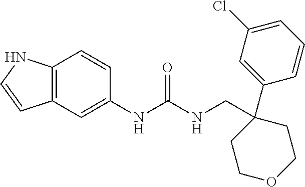 [SMILES: O=C(NCC1(c2cccc(Cl)c2)CCOCC1)Nc1ccc2[nH]ccc2c1] |
| 275 | 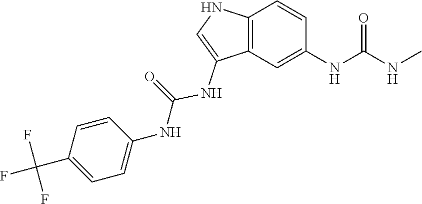 [SMILES: CNC(=O)Nc1ccc2[nH]cc(NC(=O)Nc3ccc(C(F)(F)F)cc3)c2c1] |
| 276 | 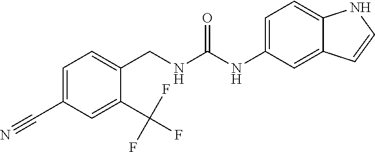 [SMILES: N#Cc1ccc(CNC(=O)Nc2ccc3[nH]ccc3c2)c(C(F)(F)F)c1] |
| 277 | 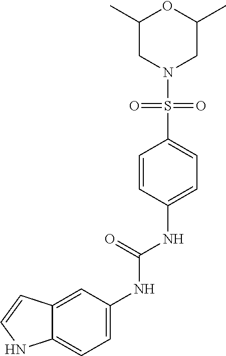 [SMILES: CC1CN(S(=O)(=O)c2ccc(NC(=O)Nc3ccc4[nH]ccc4c3)cc2)CC(C)O1] |
| 278 | 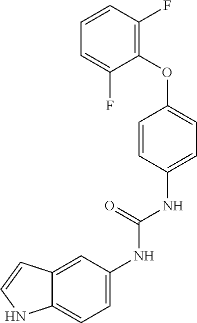 [SMILES: O=C(Nc1ccc(Oc2c(F)cccc2F)cc1)Nc1ccc2[nH]ccc2c1] |
| 279 | 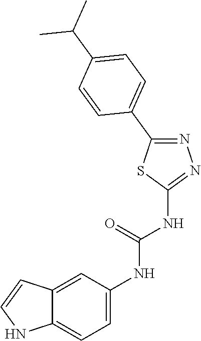 [SMILES: CC(C)c1ccc(-c2nnc(NC(=O)Nc3ccc4[nH]ccc4c3)s2)cc1] |
| 280 | 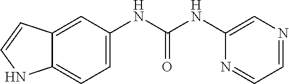 [SMILES: O=C(Nc1ccc2[nH]ccc2c1)Nc1cnccn1] |
| 281 | 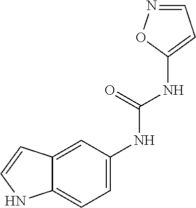 [SMILES: O=C(Nc1ccc2[nH]ccc2c1)Nc1ccno1] |
| 282 | 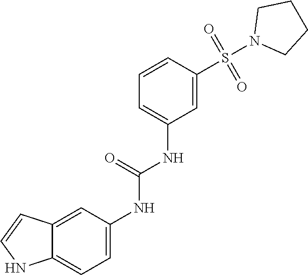 [SMILES: O=C(Nc1cccc(S(=O)(=O)N2CCCC2)c1)Nc1ccc2[nH]ccc2c1] |
| 283 | 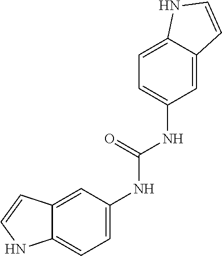 [SMILES: O=C(Nc1ccc2[nH]ccc2c1)Nc1ccc2[nH]ccc2c1] |
| 284 | 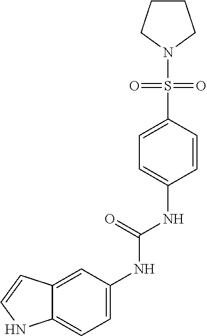 [SMILES: O=C(Nc1ccc(S(=O)(=O)N2CCCC2)cc1)Nc1ccc2[nH]ccc2c1] |
| 285 | 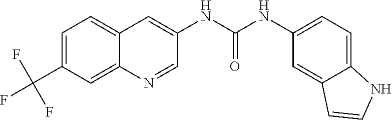 [SMILES: O=C(Nc1cnc2cc(C(F)(F)F)ccc2c1)Nc1ccc2[nH]ccc2c1] |
| 286 | 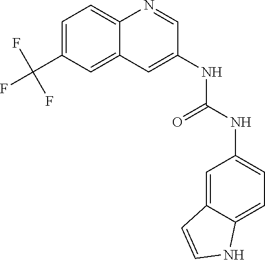 [SMILES: O=C(Nc1cnc2ccc(C(F)(F)F)cc2c1)Nc1ccc2[nH]ccc2c1] |
| 287 | 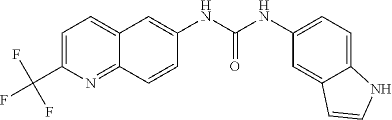 [SMILES: O=C(Nc1ccc2nc(C(F)(F)F)ccc2c1)Nc1ccc2[nH]ccc2c1] |
| 288 | 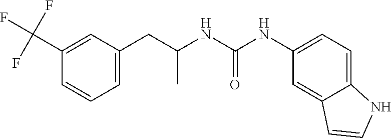 [SMILES: CC(Cc1cccc(C(F)(F)F)c1)NC(=O)Nc1ccc2[nH]ccc2c1] |
| 289 | 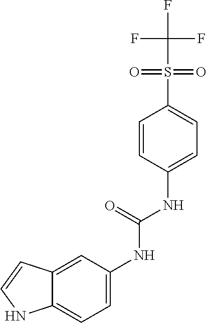 [SMILES: O=C(Nc1ccc(S(=O)(=O)C(F)(F)F)cc1)Nc1ccc2[nH]ccc2c1] |
| 290 | 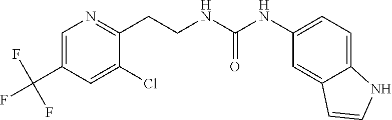 [SMILES: O=C(NCCc1ncc(C(F)(F)F)cc1Cl)Nc1ccc2[nH]ccc2c1] |
| 291 | 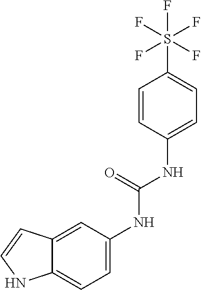 [SMILES: O=C(Nc1ccc(S(F)(F)(F)(F)F)cc1)Nc1ccc2[nH]ccc2c1] |
| 292 | 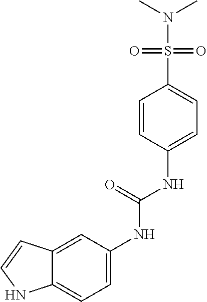 [SMILES: CN(C)S(=O)(=O)c1ccc(NC(=O)Nc2ccc3[nH]ccc3c2)cc1] |
| 293 | 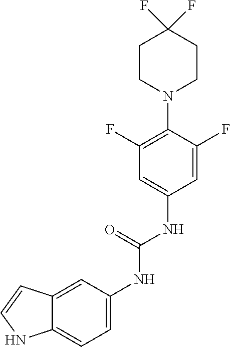 [SMILES: O=C(Nc1cc(F)c(N2CCC(F)(F)CC2)c(F)c1)Nc1ccc2[nH]ccc2c1] |
| 294 | 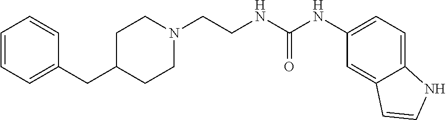 [SMILES: O=C(NCCN1CCC(Cc2ccccc2)CC1)Nc1ccc2[nH]ccc2c1] |
| 295 | 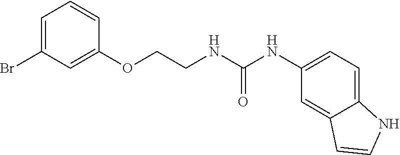 [SMILES: O=C(NCCOc1cccc(Br)c1)Nc1ccc2[nH]ccc2c1] |
| 296 | 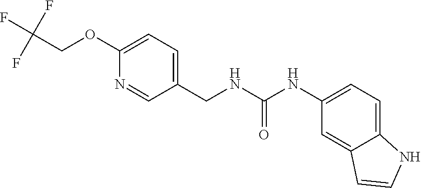 [SMILES: O=C(NCc1ccc(OCC(F)(F)F)nc1)Nc1ccc2[nH]ccc2c1] |
| 297 | 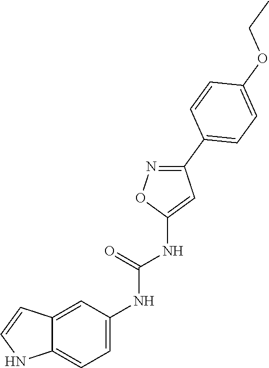 [SMILES: CCOc1ccc(-c2cc(NC(=O)Nc3ccc4[nH]ccc4c3)on2)cc1] |
| 298 | 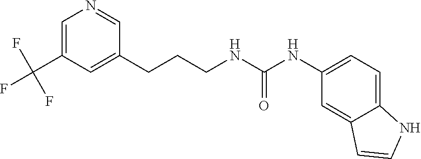 [SMILES: O=C(NCCCc1cncc(C(F)(F)F)c1)Nc1ccc2[nH]ccc2c1] |
| 299 | 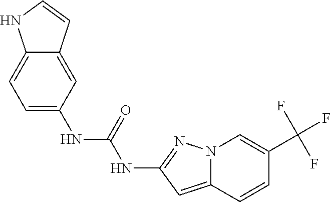 [SMILES: O=C(Nc1ccc2[nH]ccc2c1)Nc1cc2ccc(C(F)(F)F)cn2n1] |
| 300 | 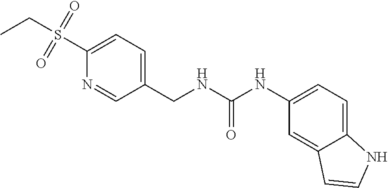 [SMILES: CCS(=O)(=O)c1ccc(CNC(=O)Nc2ccc3[nH]ccc3c2)cn1] |
| 301 | 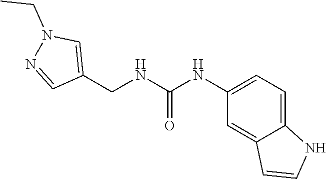 [SMILES: CCn1cc(CNC(=O)Nc2ccc3[nH]ccc3c2)cn1] |
| 302 | 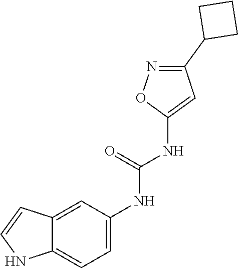 [SMILES: O=C(Nc1ccc2[nH]ccc2c1)Nc1cc(C2CCC2)no1] |
| 303 | 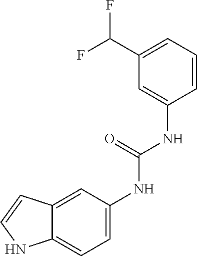 [SMILES: O=C(Nc1cccc(C(F)F)c1)Nc1ccc2[nH]ccc2c1] |
| 304 | 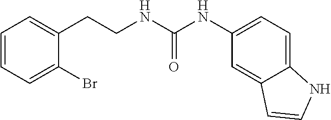 [SMILES: O=C(NCCc1ccccc1Br)Nc1ccc2[nH]ccc2c1] |
| 305 | 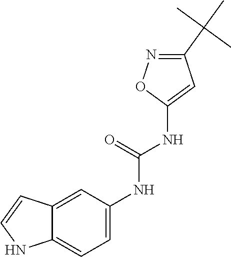 [SMILES: CC(C)(C)c1cc(NC(=O)Nc2ccc3[nH]ccc3c2)on1] |
| 306 | 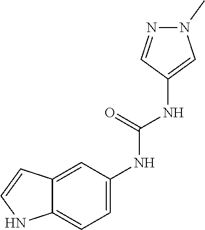 [SMILES: Cn1cc(NC(=O)Nc2ccc3[nH]ccc3c2)cn1] |
| 307 | 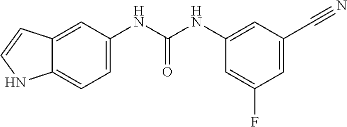 [SMILES: N#Cc1cc(F)cc(NC(=O)Nc2ccc3[nH]ccc3c2)c1] |
| 308 | 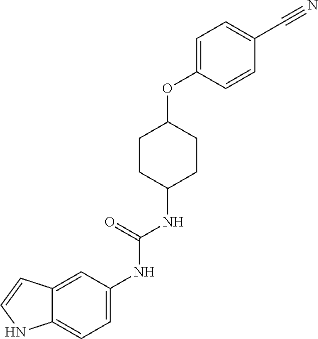 [SMILES: N#Cc1ccc(OC2CCC(NC(=O)Nc3ccc4[nH]ccc4c3)CC2)cc1] |
| 309 | 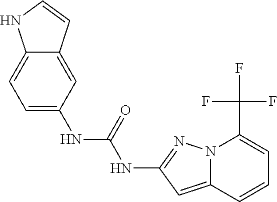 [SMILES: O=C(Nc1ccc2[nH]ccc2c1)Nc1cc2cccc(C(F)(F)F)n2n1] |
| 311 | 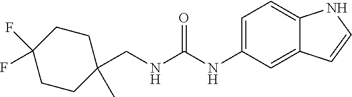 [SMILES: CC1(CNC(=O)Nc2ccc3[nH]ccc3c2)CCC(F)(F)CC1] |
| 312 | 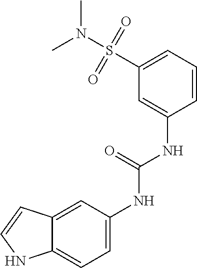 [SMILES: CN(C)S(=O)(=O)c1cccc(NC(=O)Nc2ccc3[nH]ccc3c2)c1] |
| 313 | 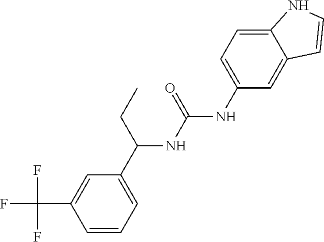 [SMILES: CCC(NC(=O)Nc1ccc2[nH]ccc2c1)c1cccc(C(F)(F)F)c1] |
| 314 | 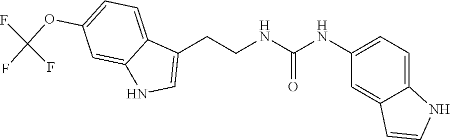 [SMILES: O=C(NCCc1c[nH]c2cc(OC(F)(F)F)ccc12)Nc1ccc2[nH]ccc2c1] |
| 315 | 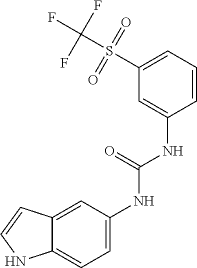 [SMILES: O=C(Nc1cccc(S(=O)(=O)C(F)(F)F)c1)Nc1ccc2[nH]ccc2c1] |
| 316 | 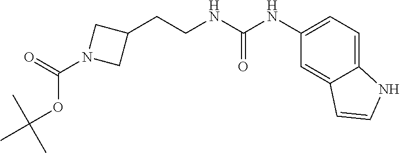 [SMILES: CC(C)(C)OC(=O)N1CC(CCNC(=O)Nc2ccc3[nH]ccc3c2)C1] |
| 317 | 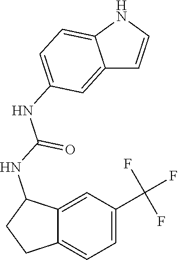 [SMILES: O=C(Nc1ccc2[nH]ccc2c1)NC1CCc2ccc(C(F)(F)F)cc21] |
| 318 | 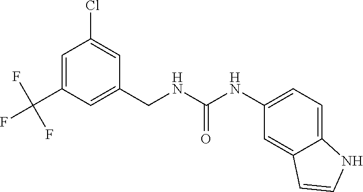 [SMILES: O=C(NCc1cc(Cl)cc(C(F)(F)F)c1)Nc1ccc2[nH]ccc2c1] |
| 319 | 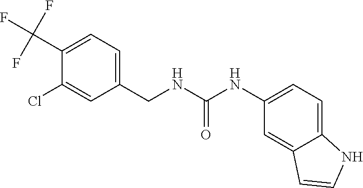 [SMILES: O=C(NCc1ccc(C(F)(F)F)c(Cl)c1)Nc1ccc2[nH]ccc2c1] |
| 320 | 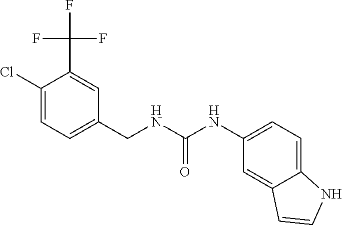 [SMILES: O=C(NCc1ccc(Cl)c(C(F)(F)F)c1)Nc1ccc2[nH]ccc2c1] |
| 321 | 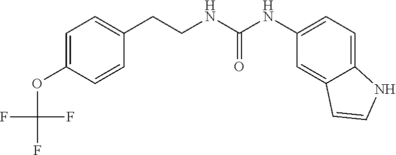 [SMILES: O=C(NCCc1ccc(OC(F)(F)F)cc1)Nc1ccc2[nH]ccc2c1] |
| 322 | 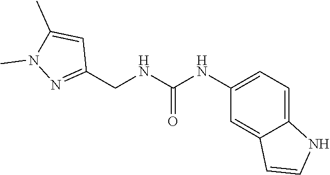 [SMILES: Cc1cc(CNC(=O)Nc2ccc3[nH]ccc3c2)nn1C] |
| 323 | 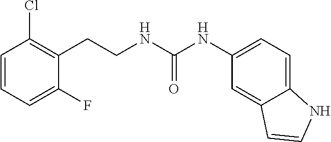 [SMILES: O=C(NCCc1c(F)cccc1Cl)Nc1ccc2[nH]ccc2c1] |
| 324 | 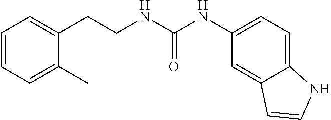 [SMILES: Cc1ccccc1CCNC(=O)Nc1ccc2[nH]ccc2c1] |
| 325 | 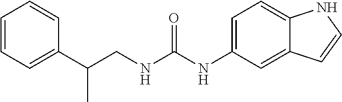 [SMILES: CC(CNC(=O)Nc1ccc2[nH]ccc2c1)c1ccccc1] |
| 326 | 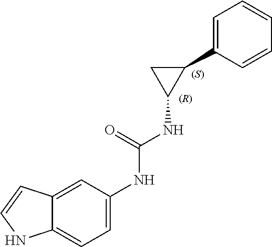 |
| 327 | 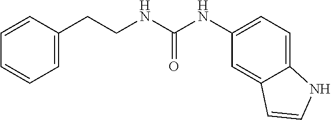 [SMILES: O=C(NCCc1ccccc1)Nc1ccc2[nH]ccc2c1] |
| 328 | 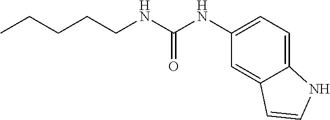 [SMILES: CCCCCNC(=O)Nc1ccc2[nH]ccc2c1] |
| 329 | 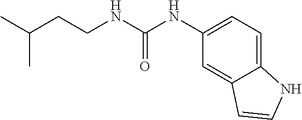 [SMILES: CC(C)CCNC(=O)Nc1ccc2[nH]ccc2c1] |
| 330 | 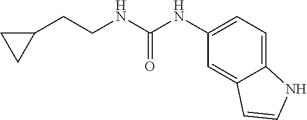 [SMILES: O=C(NCCC1CC1)Nc1ccc2[nH]ccc2c1] |
| 331 | 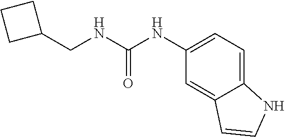 [SMILES: O=C(NCC1CCC1)Nc1ccc2[nH]ccc2c1] |
| 332 | 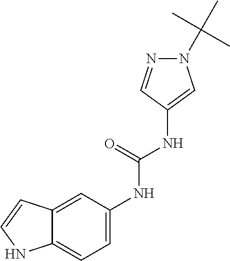 [SMILES: CC(C)(C)n1cc(NC(=O)Nc2ccc3[nH]ccc3c2)cn1] |
| 333 | 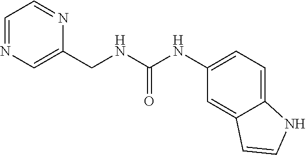 [SMILES: O=C(NCc1cnccn1)Nc1ccc2[nH]ccc2c1] |
| 334 | 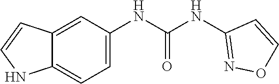 [SMILES: O=C(Nc1ccc2[nH]ccc2c1)Nc1ccon1] |
| 335 | 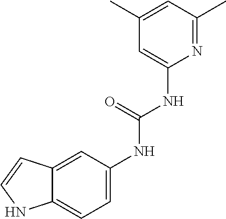 [SMILES: Cc1cc(C)nc(NC(=O)Nc2ccc3[nH]ccc3c2)c1] |
| 336 | 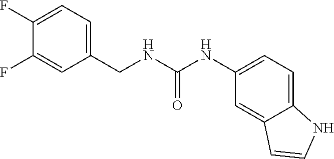 [SMILES: O=C(NCc1ccc(F)c(F)c1)Nc1ccc2[nH]ccc2c1] |
| 337 | 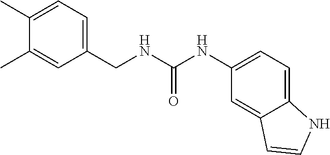 [SMILES: Cc1ccc(CNC(=O)Nc2ccc3[nH]ccc3c2)cc1C] |
| 338 | 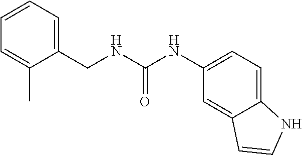 [SMILES: Cc1ccccc1CNC(=O)Nc1ccc2[nH]ccc2c1] |
| 339 | 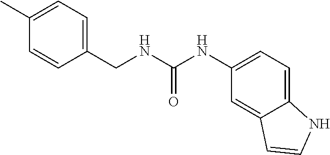 [SMILES: Cc1ccc(CNC(=O)Nc2ccc3[nH]ccc3c2)cc1] |
| 340 | 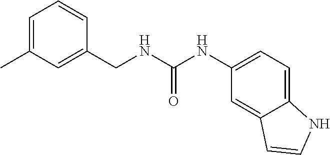 [SMILES: Cc1cccc(CNC(=O)Nc2ccc3[nH]ccc3c2)c1] |
| 341 | 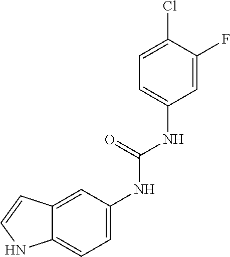 [SMILES: O=C(Nc1ccc(Cl)c(F)c1)Nc1ccc2[nH]ccc2c1] |
| 342 | 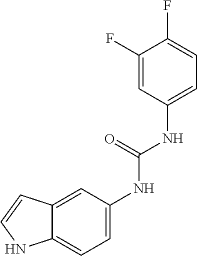 [SMILES: O=C(Nc1ccc(F)c(F)c1)Nc1ccc2[nH]ccc2c1] |
| 343 | 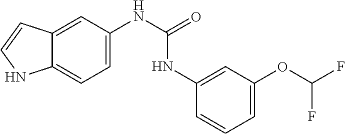 [SMILES: O=C(Nc1cccc(OC(F)F)c1)Nc1ccc2[nH]ccc2c1] |
| 345 | 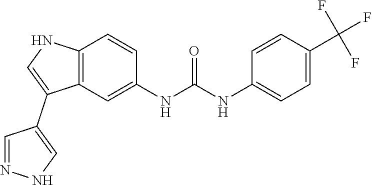 [SMILES: O=C(Nc1ccc(C(F)(F)F)cc1)Nc1ccc2[nH]cc(-c3cn[nH]c3)c2c1] |
| 346 | 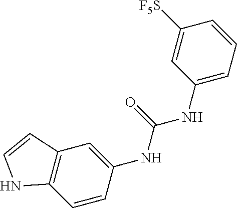 |
| 347 | 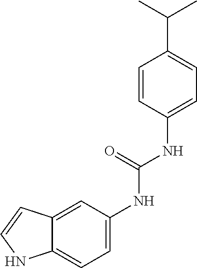 [SMILES: CC(C)c1ccc(NC(=O)Nc2ccc3[nH]ccc3c2)cc1] |
| 348 | 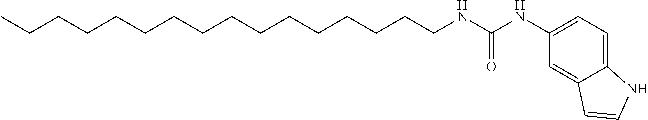 [SMILES: CCCCCCCCCCCCCCCCNC(=O)Nc1ccc2[nH]ccc2c1] |
| 349 | 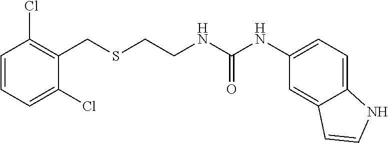 [SMILES: O=C(NCCSCc1c(Cl)cccc1Cl)Nc1ccc2[nH]ccc2c1] |
| 350 | 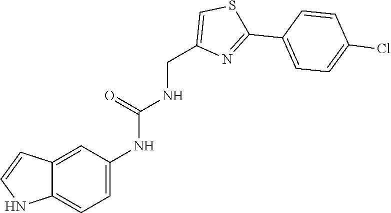 [SMILES: O=C(NCc1csc(-c2ccc(Cl)cc2)n1)Nc1ccc2[nH]ccc2c1] |
| 352 | 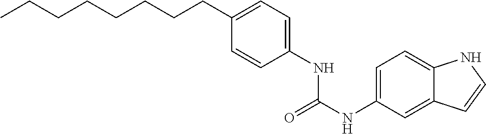 [SMILES: CCCCCCCCc1ccc(NC(=O)Nc2ccc3[nH]ccc3c2)cc1] |
| 351 | 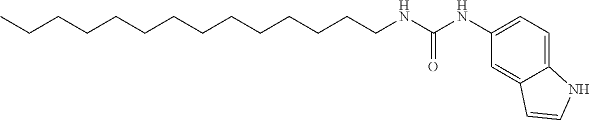 [SMILES: CCCCCCCCCCCCCCNC(=O)Nc1ccc2[nH]ccc2c1] |
| 353 | 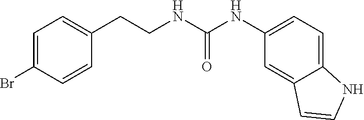 [SMILES: O=C(NCCc1ccc(Br)cc1)Nc1ccc2[nH]ccc2c1] |
| 354 | 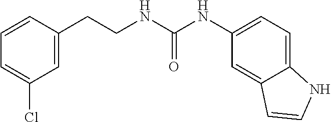 [SMILES: O=C(NCCc1cccc(Cl)c1)Nc1ccc2[nH]ccc2c1] |
| 355 | 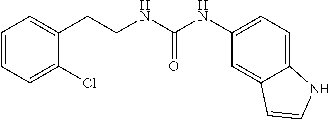 [SMILES: O=C(NCCc1ccccc1Cl)Nc1ccc2[nH]ccc2c1] |
| 356 | 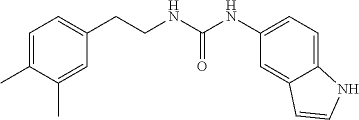 [SMILES: Cc1ccc(CCNC(=O)Nc2ccc3[nH]ccc3c2)cc1C] |
| 357 | 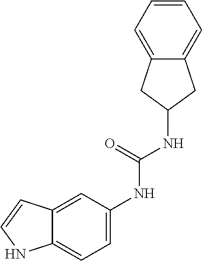 [SMILES: O=C(Nc1ccc2[nH]ccc2c1)NC1Cc2ccccc2C1] |
| 358 | 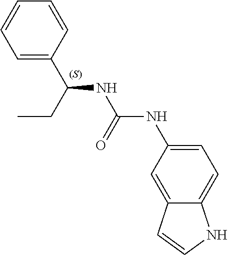 |
| 359 | 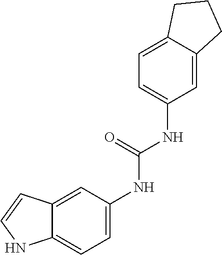 [SMILES: O=C(Nc1ccc2c(c1)CCC2)Nc1ccc2[nH]ccc2c1] |
| 360 | 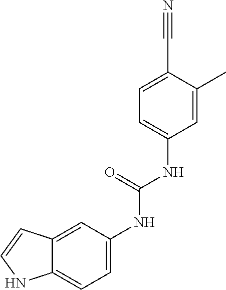 [SMILES: Cc1cc(NC(=O)Nc2ccc3[nH]ccc3c2)ccc1C#N] |
| 361 | 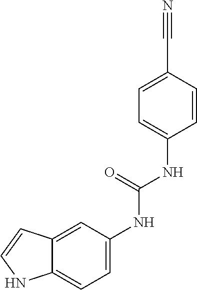 [SMILES: N#Cc1ccc(NC(=O)Nc2ccc3[nH]ccc3c2)cc1] |
| 362 | 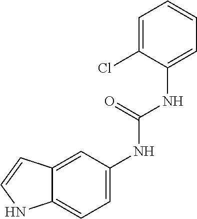 [SMILES: O=C(Nc1ccc2[nH]ccc2c1)Nc1ccccc1Cl] |
| 363 | 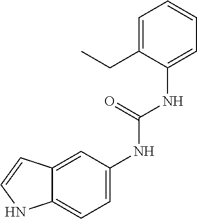 [SMILES: CCc1ccccc1NC(=O)Nc1ccc2[nH]ccc2c1] |
| 364 | 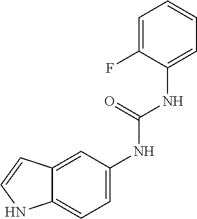 [SMILES: O=C(Nc1ccc2[nH]ccc2c1)Nc1ccccc1F] |
| 365 | 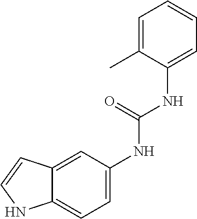 [SMILES: Cc1ccccc1NC(=O)Nc1ccc2[nH]ccc2c1] |
| 366 | 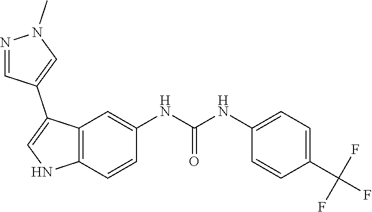 [SMILES: Cn1cc(-c2c[nH]c3ccc(NC(=O)Nc4ccc(C(F)(F)F)cc4)cc23)cn1] |
| 370 | 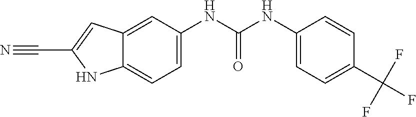 [SMILES: N#Cc1cc2cc(NC(=O)Nc3ccc(C(F)(F)F)cc3)ccc2[nH]1] |
| 371 | 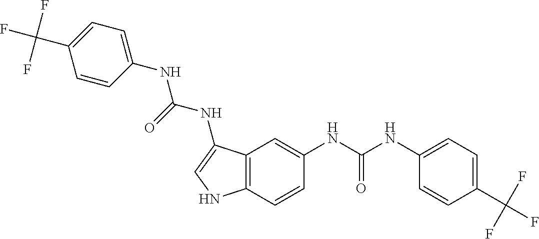 [SMILES: O=C(Nc1ccc(C(F)(F)F)cc1)Nc1ccc2[nH]cc(NC(=O)Nc3ccc(C(F)(F)F)cc3)c2c1] |
| 372 | 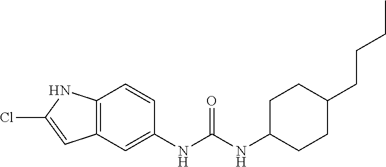 [SMILES: CCCCC1CCC(NC(=O)Nc2ccc3[nH]c(Cl)cc3c2)CC1] |
| 373 | 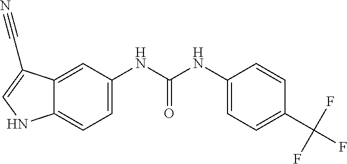 [SMILES: N#Cc1c[nH]c2ccc(NC(=O)Nc3ccc(C(F)(F)F)cc3)cc12] |
| 374 | 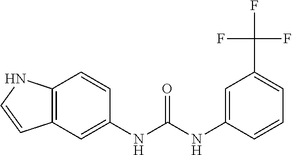 [SMILES: O=C(Nc1cccc(C(F)(F)F)c1)Nc1ccc2[nH]ccc2c1] |
| 375 | 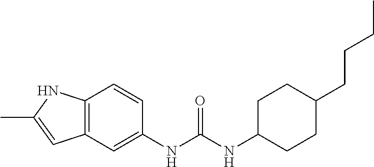 [SMILES: CCCCC1CCC(NC(=O)Nc2ccc3[nH]c(C)cc3c2)CC1] |
| 376 | 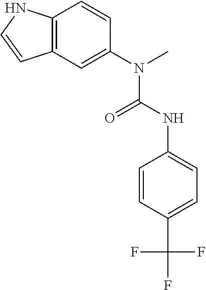 [SMILES: CN(C(=O)Nc1ccc(C(F)(F)F)cc1)c1ccc2[nH]ccc2c1] |
| 377 | 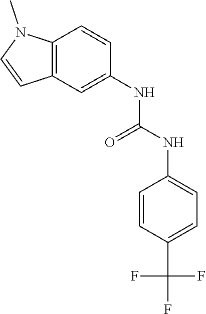 [SMILES: Cn1ccc2cc(NC(=O)Nc3ccc(C(F)(F)F)cc3)ccc21] |
| 378 | 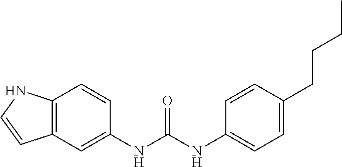 [SMILES: CCCCc1ccc(NC(=O)Nc2ccc3[nH]ccc3c2)cc1] |
| 379 | 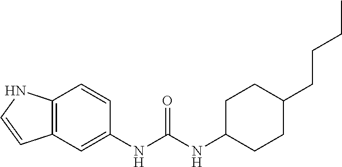 [SMILES: CCCCC1CCC(NC(=O)Nc2ccc3[nH]ccc3c2)CC1] |
| 380 | 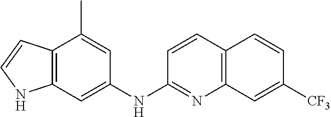 [SMILES: Cc1cc(Nc2ccc3ccc(C(F)(F)F)cc3n2)cc2[nH]ccc12] |
| 381 | 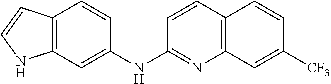 [SMILES: FC(F)(F)c1ccc2ccc(Nc3ccc4cc[nH]c4c3)nc2c1] |
| 382 | 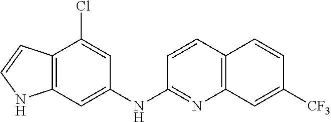 [SMILES: FC(F)(F)c1ccc2ccc(Nc3cc(Cl)c4cc[nH]c4c3)nc2c1] |
| 383 | 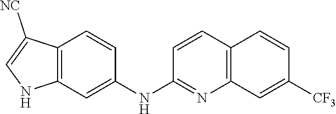 [SMILES: N#Cc1c[nH]c2cc(Nc3ccc4ccc(C(F)(F)F)cc4n3)ccc12] |
| 384 | 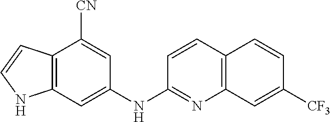 [SMILES: N#Cc1cc(Nc2ccc3ccc(C(F)(F)F)cc3n2)cc2[nH]ccc12] |
| 385 | 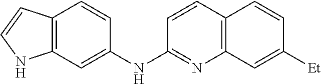 [SMILES: CCc1ccc2ccc(Nc3ccc4cc[nH]c4c3)nc2c1] |
| 386 | 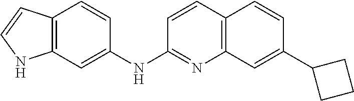 [SMILES: c1cc2ccc(Nc3ccc4ccc(C5CCC5)cc4n3)cc2[nH]1] |
| 387 | 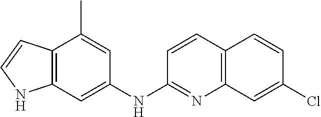 [SMILES: Cc1cc(Nc2ccc3ccc(Cl)cc3n2)cc2[nH]ccc12] |
| 388 | 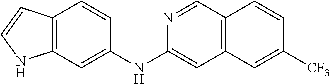 [SMILES: FC(F)(F)c1ccc2cnc(Nc3ccc4cc[nH]c4c3)cc2c1] |
| 389 | 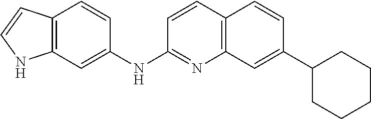 [SMILES: c1cc2ccc(Nc3ccc4ccc(C5CCCCC5)cc4n3)cc2[nH]1] |
| 390 | 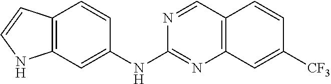 [SMILES: FC(F)(F)c1ccc2cnc(Nc3ccc4cc[nH]c4c3)nc2c1] |
| 391 | 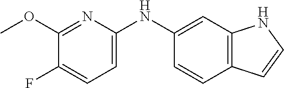 [SMILES: COc1nc(Nc2ccc3cc[nH]c3c2)ccc1F] |
| 392 | 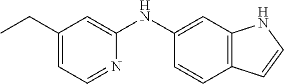 [SMILES: CCc1ccnc(Nc2ccc3cc[nH]c3c2)c1] |
| 393 | 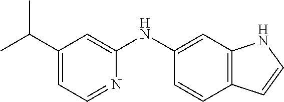 [SMILES: CC(C)c1ccnc(Nc2ccc3cc[nH]c3c2)c1] |
| 394 | 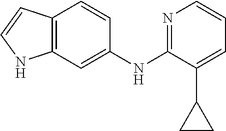 [SMILES: c1cnc(Nc2ccc3cc[nH]c3c2)c(C2CC2)c1] |
| 395 | 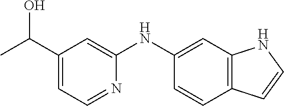 [SMILES: CC(O)c1ccnc(Nc2ccc3cc[nH]c3c2)c1] |
| 396 | 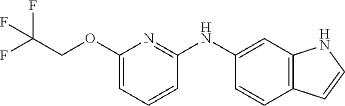 [SMILES: FC(F)(F)COc1cccc(Nc2ccc3cc[nH]c3c2)n1] |
| 397 | 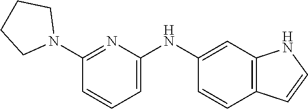 [SMILES: c1cc(Nc2ccc3cc[nH]c3c2)nc(N2CCCC2)c1] |
| 398 | 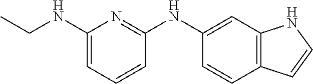 [SMILES: CCNc1cccc(Nc2ccc3cc[nH]c3c2)n1] |
| 399 | 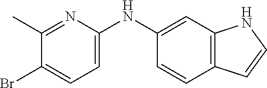 [SMILES: Cc1nc(Nc2ccc3cc[nH]c3c2)ccc1Br] |
| 400 | 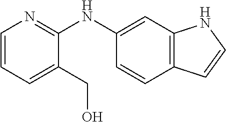 [SMILES: OCc1cccnc1Nc1ccc2cc[nH]c2c1] |
| 401 | 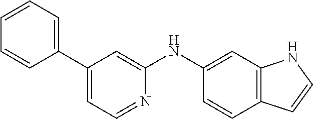 [SMILES: c1ccc(-c2ccnc(Nc3ccc4cc[nH]c4c3)c2)cc1] |
| 402 | 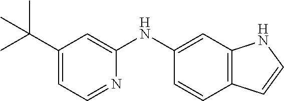 [SMILES: CC(C)(C)c1ccnc(Nc2ccc3cc[nH]c3c2)c1] |
| 403 | 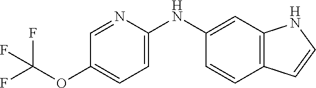 [SMILES: FC(F)(F)Oc1ccc(Nc2ccc3cc[nH]c3c2)nc1] |
| 404 | 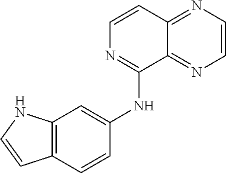 [SMILES: c1cc2nccnc2c(Nc2ccc3cc[nH]c3c2)n1] |
| 405 | 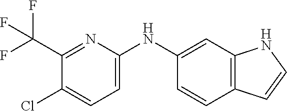 [SMILES: FC(F)(F)c1nc(Nc2ccc3cc[nH]c3c2)ccc1Cl] |
| 406 | 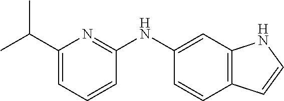 [SMILES: CC(C)c1cccc(Nc2ccc3cc[nH]c3c2)n1] |
| 407 | 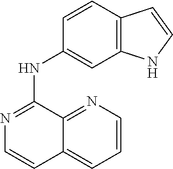 [SMILES: c1cnc2c(Nc3ccc4cc[nH]c4c3)nccc2c1] |
| 408 | 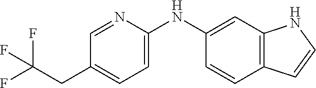 [SMILES: FC(F)(F)Cc1ccc(Nc2ccc3cc[nH]c3c2)nc1] |
| 409 | 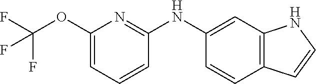 [SMILES: FC(F)(F)Oc1cccc(Nc2ccc3cc[nH]c3c2)n1] |
| 410 | 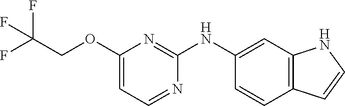 [SMILES: FC(F)(F)COc1ccnc(Nc2ccc3cc[nH]c3c2)n1] |
| 411 | 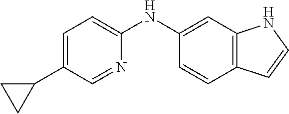 [SMILES: c1cc2ccc(Nc3ccc(C4CC4)cn3)cc2[nH]1] |
| 412 | 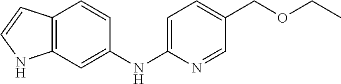 [SMILES: CCOCc1ccc(Nc2ccc3cc[nH]c3c2)nc1] |
| 413 | 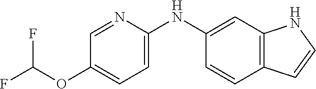 [SMILES: FC(F)Oc1ccc(Nc2ccc3cc[nH]c3c2)nc1] |
| 414 | 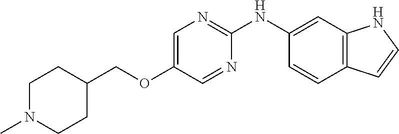 [SMILES: CN1CCC(COc2cnc(Nc3ccc4cc[nH]c4c3)nc2)CC1] |
| 415 | 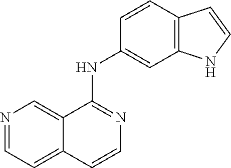 [SMILES: c1cc2ccnc(Nc3ccc4cc[nH]c4c3)c2cn1] |
| 416 | 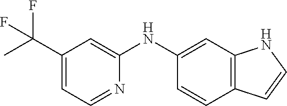 [SMILES: CC(F)(F)c1ccnc(Nc2ccc3cc[nH]c3c2)c1] |
| 417 | 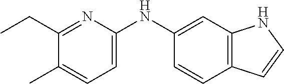 [SMILES: CCc1nc(Nc2ccc3cc[nH]c3c2)ccc1C] |
| 418 | 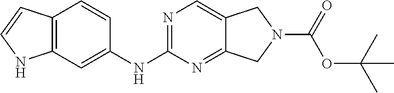 [SMILES: CC(C)(C)OC(=O)N1Cc2cnc(Nc3ccc4cc[nH]c4c3)nc2C1] |
| 419 | 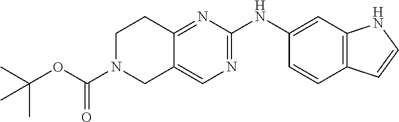 [SMILES: CC(C)(C)OC(=O)N1CCc2nc(Nc3ccc4cc[nH]c4c3)ncc2C1] |
| 420 | 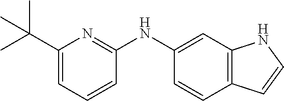 [SMILES: CC(C)(C)c1cccc(Nc2ccc3cc[nH]c3c2)n1] |
| 421 | 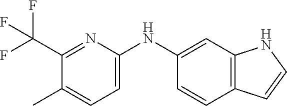 [SMILES: Cc1ccc(Nc2ccc3cc[nH]c3c2)nc1C(F)(F)F] |
| 422 | 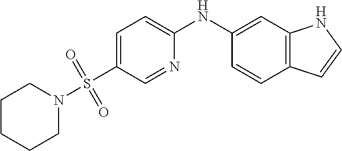 [SMILES: O=S(=O)(c1ccc(Nc2ccc3cc[nH]c3c2)nc1)N1CCCCC1] |
| 423 | 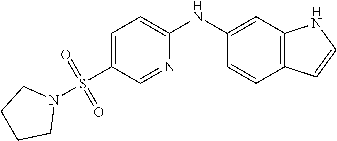 [SMILES: O=S(=O)(c1ccc(Nc2ccc3cc[nH]c3c2)nc1)N1CCCC1] |
| 424 | 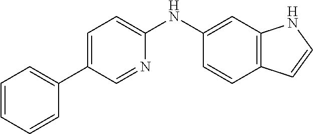 [SMILES: c1ccc(-c2ccc(Nc3ccc4cc[nH]c4c3)nc2)cc1] |
| 425 | 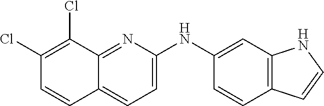 [SMILES: Clc1ccc2ccc(Nc3ccc4cc[nH]c4c3)nc2c1Cl] |
| 426 | 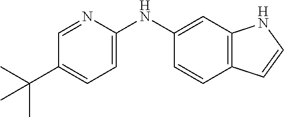 [SMILES: CC(C)(C)c1ccc(Nc2ccc3cc[nH]c3c2)nc1] |
| 427 | 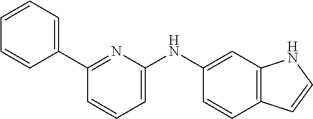 [SMILES: c1ccc(-c2cccc(Nc3ccc4cc[nH]c4c3)n2)cc1] |
| 428 | 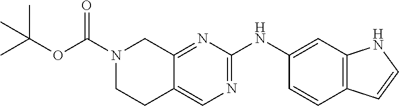 [SMILES: CC(C)(C)OC(=O)N1CCc2cnc(Nc3ccc4cc[nH]c4c3)nc2C1] |
| 429 | 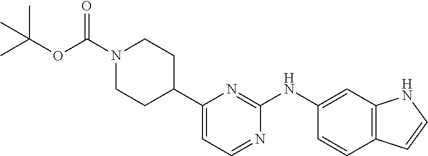 [SMILES: CC(C)(C)OC(=O)N1CCC(c2ccnc(Nc3ccc4cc[nH]c4c3)n2)CC1] |
| 430 | 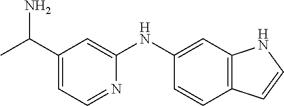 [SMILES: CC(N)c1ccnc(Nc2ccc3cc[nH]c3c2)c1] |
| 431 | 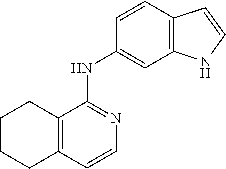 [SMILES: c1cc2c(c(Nc3ccc4cc[nH]c4c3)n1)CCCC2] |
| 432 | 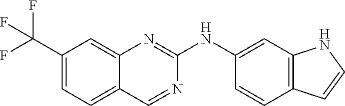 [SMILES: FC(F)(F)c1ccc2cnc(Nc3ccc4cc[nH]c4c3)nc2c1] |
| 433 | 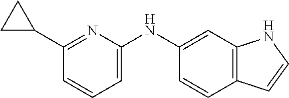 [SMILES: c1cc(Nc2ccc3cc[nH]c3c2)nc(C2CC2)c1] |
| 434 | 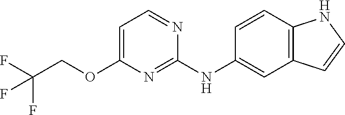 [SMILES: FC(F)(F)COc1ccnc(Nc2ccc3[nH]ccc3c2)n1] |
| 435 | 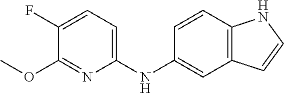 [SMILES: COc1nc(Nc2ccc3[nH]ccc3c2)ccc1F] |
| 436 | 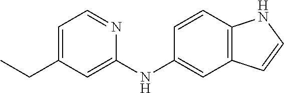 [SMILES: CCc1ccnc(Nc2ccc3[nH]ccc3c2)c1] |
| 437 | 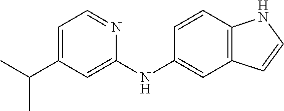 [SMILES: CC(C)c1ccnc(Nc2ccc3[nH]ccc3c2)c1] |
| 438 | 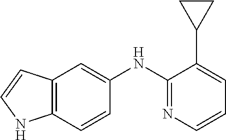 [SMILES: c1cnc(Nc2ccc3[nH]ccc3c2)c(C2CC2)c1] |
| 439 | 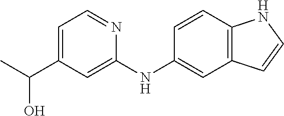 [SMILES: CC(O)c1ccnc(Nc2ccc3[nH]ccc3c2)c1] |
| 440 | 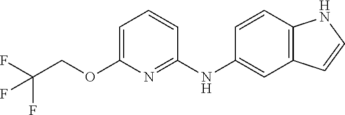 [SMILES: FC(F)(F)COc1cccc(Nc2ccc3[nH]ccc3c2)n1] |
| 441 | 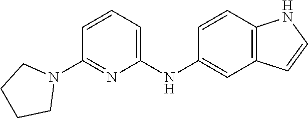 [SMILES: c1cc(Nc2ccc3[nH]ccc3c2)nc(N2CCCC2)c1] |
| 442 | 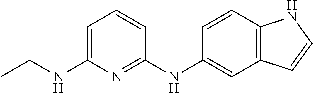 [SMILES: CCNc1cccc(Nc2ccc3[nH]ccc3c2)n1] |
| 443 | 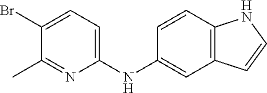 [SMILES: Cc1nc(Nc2ccc3[nH]ccc3c2)ccc1Br] |
| 444 | 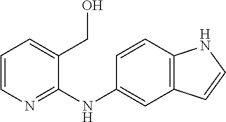 [SMILES: OCc1cccnc1Nc1ccc2[nH]ccc2c1] |
| 445 | 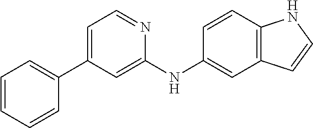 [SMILES: c1ccc(-c2ccnc(Nc3ccc4[nH]ccc4c3)c2)cc1] |
| 446 | 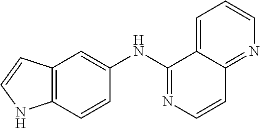 [SMILES: c1cnc2ccnc(Nc3ccc4[nH]ccc4c3)c2c1] |
| 447 | 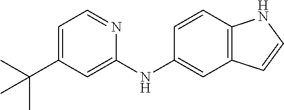 [SMILES: CC(C)(C)c1ccnc(Nc2ccc3[nH]ccc3c2)c1] |
| 448 | 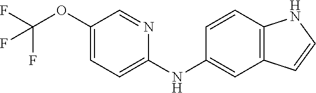 [SMILES: FC(F)(F)Oc1ccc(Nc2ccc3[nH]ccc3c2)nc1] |
| 449 | 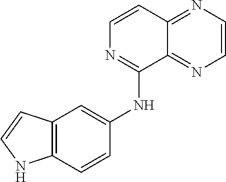 [SMILES: c1cc2nccnc2c(Nc2ccc3[nH]ccc3c2)n1] |
| 450 | 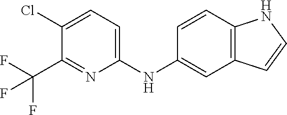 [SMILES: FC(F)(F)c1nc(Nc2ccc3[nH]ccc3c2)ccc1Cl] |
| 451 | 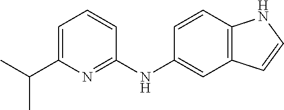 [SMILES: CC(C)c1cccc(Nc2ccc3[nH]ccc3c2)n1] |
| 452 | 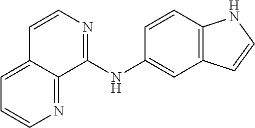 [SMILES: c1cnc2c(Nc3ccc4[nH]ccc4c3)nccc2c1] |
| 453 | 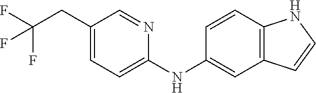 [SMILES: FC(F)(F)Cc1ccc(Nc2ccc3[nH]ccc3c2)nc1] |
| 454 | 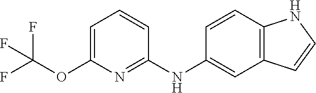 [SMILES: FC(F)(F)Oc1cccc(Nc2ccc3[nH]ccc3c2)n1] |
| 455 | 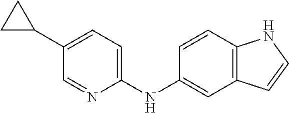 [SMILES: c1cc2cc(Nc3ccc(C4CC4)cn3)ccc2[nH]1] |
| 456 | 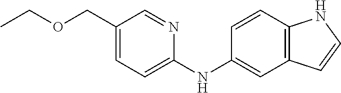 [SMILES: CCOCc1ccc(Nc2ccc3[nH]ccc3c2)nc1] |
| 457 | 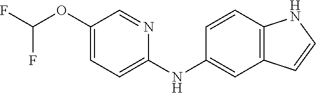 [SMILES: FC(F)Oc1ccc(Nc2ccc3[nH]ccc3c2)nc1] |
| 458 | 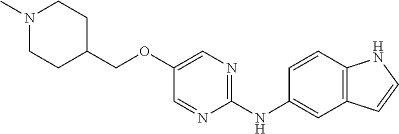 [SMILES: CN1CCC(COc2cnc(Nc3ccc4[nH]ccc4c3)nc2)CC1] |
| 459 | 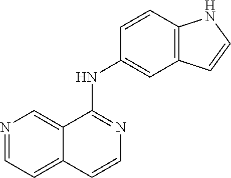 [SMILES: c1cc2ccnc(Nc3ccc4[nH]ccc4c3)c2cn1] |
| 460 | 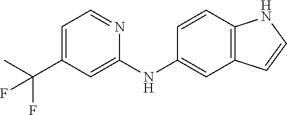 [SMILES: CC(F)(F)c1ccnc(Nc2ccc3[nH]ccc3c2)c1] |
| 461 | 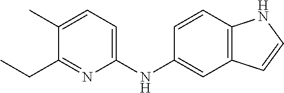 [SMILES: CCc1nc(Nc2ccc3[nH]ccc3c2)ccc1C] |
| 462 | 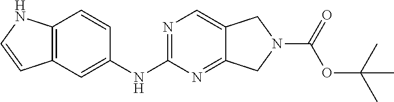 [SMILES: CC(C)(C)OC(=O)N1Cc2cnc(Nc3ccc4[nH]ccc4c3)nc2C1] |
| 463 | 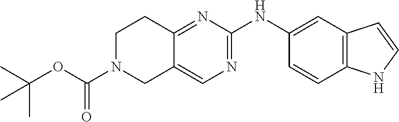 [SMILES: CC(C)(C)OC(=O)N1CCc2nc(Nc3ccc4[nH]ccc4c3)ncc2C1] |
| 464 | 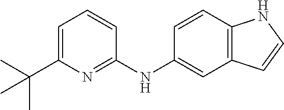 [SMILES: CC(C)(C)c1cccc(Nc2ccc3[nH]ccc3c2)n1] |
| 465 | 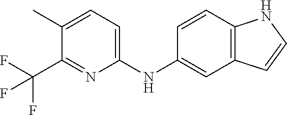 [SMILES: Cc1ccc(Nc2ccc3[nH]ccc3c2)nc1C(F)(F)F] |
| 466 | 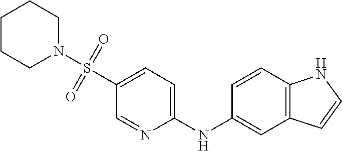 [SMILES: O=S(=O)(c1ccc(Nc2ccc3[nH]ccc3c2)nc1)N1CCCCC1] |
| 467 | 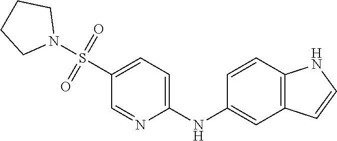 [SMILES: O=S(=O)(c1ccc(Nc2ccc3[nH]ccc3c2)nc1)N1CCCC1] |
| 468 | 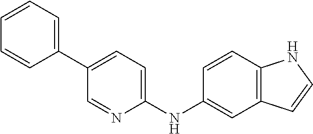 [SMILES: c1ccc(-c2ccc(Nc3ccc4[nH]ccc4c3)nc2)cc1] |
| 469 | 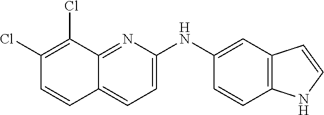 [SMILES: Clc1ccc2ccc(Nc3ccc4[nH]ccc4c3)nc2c1Cl] |
| 470 | 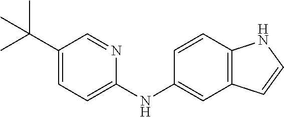 [SMILES: CC(C)(C)c1ccc(Nc2ccc3[nH]ccc3c2)nc1] |
| 471 | 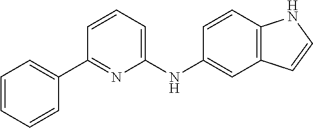 [SMILES: c1ccc(-c2cccc(Nc3ccc4[nH]ccc4c3)n2)cc1] |
| 472 | 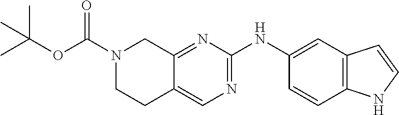 [SMILES: CC(C)(C)OC(=O)N1CCc2cnc(Nc3ccc4[nH]ccc4c3)nc2C1] |
| 473 | 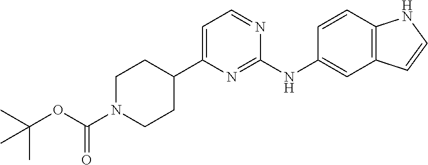 [SMILES: CC(C)(C)OC(=O)N1CCC(c2ccnc(Nc3ccc4[nH]ccc4c3)n2)CC1] |
| 474 | 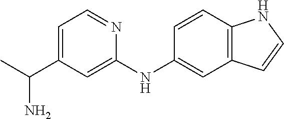 [SMILES: CC(N)c1ccnc(Nc2ccc3[nH]ccc3c2)c1] |
| 475 | 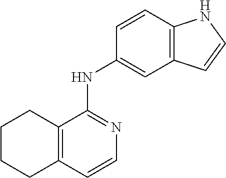 [SMILES: c1cc2c(c(Nc3ccc4[nH]ccc4c3)n1)CCCC2] |
| 476 | 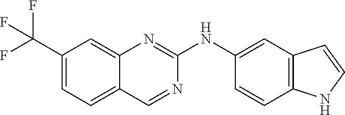 [SMILES: FC(F)(F)c1ccc2cnc(Nc3ccc4[nH]ccc4c3)nc2c1] |
| 477 | 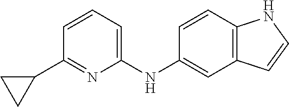 [SMILES: c1cc(Nc2ccc3[nH]ccc3c2)nc(C2CC2)c1] |
| 478 | 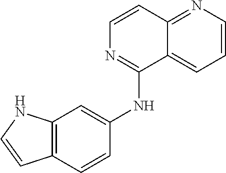 [SMILES: c1cnc2ccnc(Nc3ccc4cc[nH]c4c3)c2c1] |
| 479 | 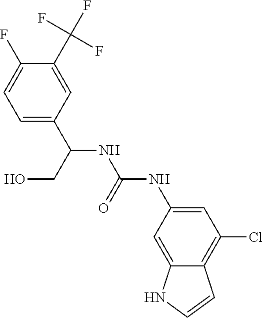 [SMILES: O=C(Nc1cc(Cl)c2cc[nH]c2c1)NC(CO)c1ccc(F)c(C(F)(F)F)c1] |
| 480 | 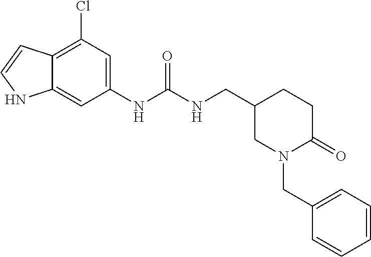 [SMILES: O=C(NCC1CCC(=O)N(Cc2ccccc2)C1)Nc1cc(Cl)c2cc[nH]c2c1] |
| 481 | 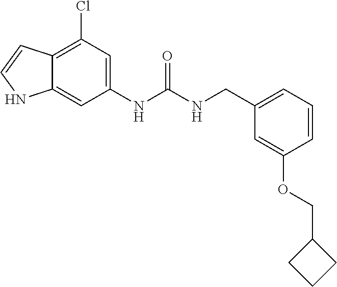 [SMILES: O=C(NCc1cccc(OCC2CCC2)c1)Nc1cc(Cl)c2cc[nH]c2c1] |
| 482 | 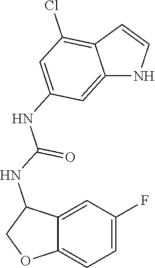 [SMILES: O=C(Nc1cc(Cl)c2cc[nH]c2c1)NC1COc2ccc(F)cc21] |
| 483 | 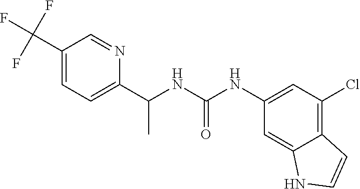 [SMILES: CC(NC(=O)Nc1cc(Cl)c2cc[nH]c2c1)c1ccc(C(F)(F)F)cn1] |
| 484 | 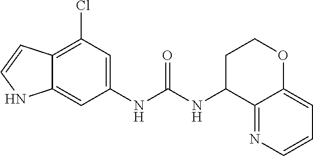 [SMILES: O=C(Nc1cc(Cl)c2cc[nH]c2c1)NC1CCOc2cccnc21] |
| 485 | 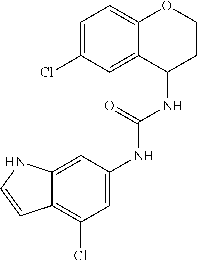 [SMILES: O=C(Nc1cc(Cl)c2cc[nH]c2c1)NC1CCOc2ccc(Cl)cc21] |
| 486 | 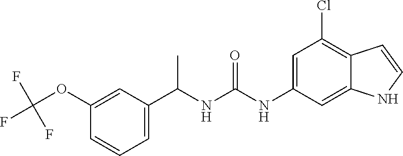 [SMILES: CC(NC(=O)Nc1cc(Cl)c2cc[nH]c2c1)c1cccc(OC(F)(F)F)c1] |
| 487 | 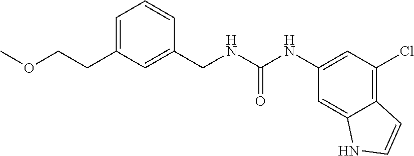 [SMILES: COCCc1cccc(CNC(=O)Nc2cc(Cl)c3cc[nH]c3c2)c1] |
| 488 | 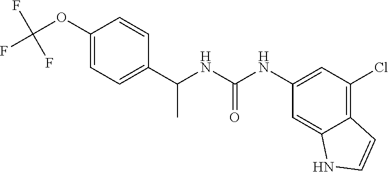 [SMILES: CC(NC(=O)Nc1cc(Cl)c2cc[nH]c2c1)c1ccc(OC(F)(F)F)cc1] |
| 489 | 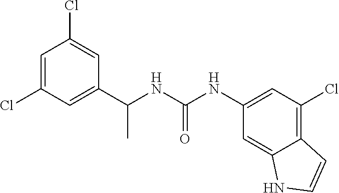 [SMILES: CC(NC(=O)Nc1cc(Cl)c2cc[nH]c2c1)c1cc(Cl)cc(Cl)c1] |
| 490 | 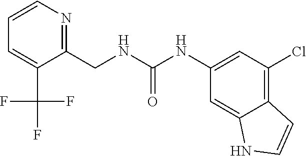 [SMILES: O=C(NCc1ncccc1C(F)(F)F)Nc1cc(Cl)c2cc[nH]c2c1] |
| 491 | 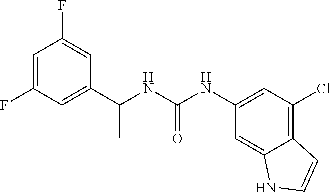 [SMILES: CC(NC(=O)Nc1cc(Cl)c2cc[nH]c2c1)c1cc(F)cc(F)c1] |
| 492 | 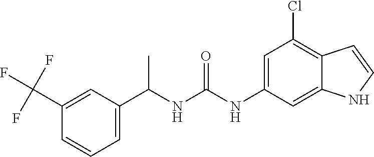 [SMILES: CC(NC(=O)Nc1cc(Cl)c2cc[nH]c2c1)c1cccc(C(F)(F)F)c1] |
| 493 | 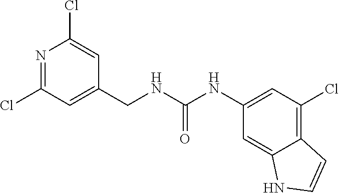 [SMILES: O=C(NCc1cc(Cl)nc(Cl)c1)Nc1cc(Cl)c2cc[nH]c2c1] |
| 494 | 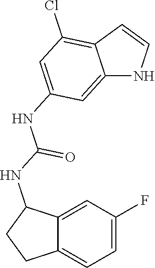 [SMILES: O=C(Nc1cc(Cl)c2cc[nH]c2c1)NC1CCc2ccc(F)cc21] |
| 495 | 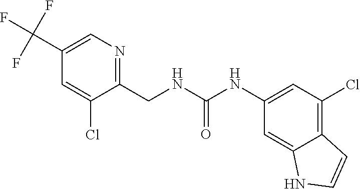 [SMILES: O=C(NCc1ncc(C(F)(F)F)cc1Cl)Nc1cc(Cl)c2cc[nH]c2c1] |
| 496 | 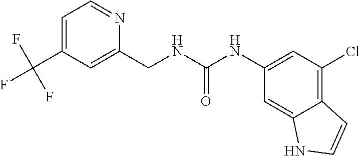 [SMILES: O=C(NCc1cc(C(F)(F)F)ccn1)Nc1cc(Cl)c2cc[nH]c2c1] |
| 497 | 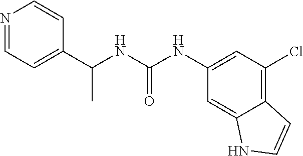 [SMILES: CC(NC(=O)Nc1cc(Cl)c2cc[nH]c2c1)c1ccncc1] |
| 498 | 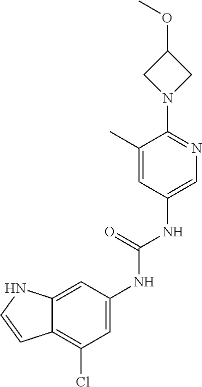 [SMILES: COC1CN(c2ncc(NC(=O)Nc3cc(Cl)c4cc[nH]c4c3)cc2C)C1] |
| 499 | 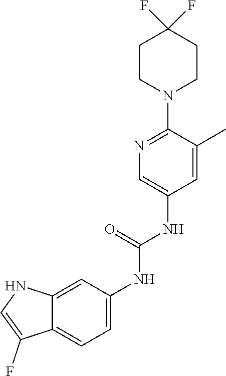 [SMILES: Cc1cc(NC(=O)Nc2ccc3c(F)c[nH]c3c2)cnc1N1CCC(F)(F)CC1] |
| 500 | 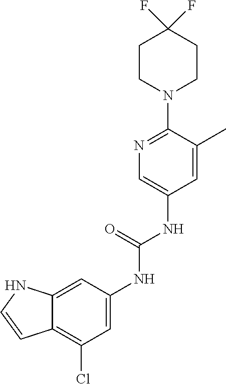 [SMILES: Cc1cc(NC(=O)Nc2cc(Cl)c3cc[nH]c3c2)cnc1N1CCC(F)(F)CC1] |
| 501 | 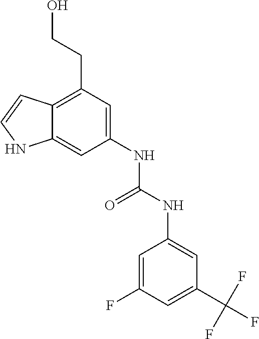 [SMILES: O=C(Nc1cc(F)cc(C(F)(F)F)c1)Nc1cc(CCO)c2cc[nH]c2c1] |
| 502 | 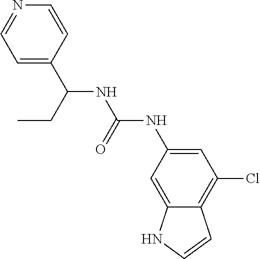 [SMILES: CCC(NC(=O)Nc1cc(Cl)c2cc[nH]c2c1)c1ccncc1] |
| 503 | 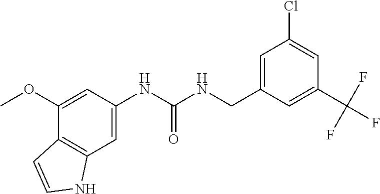 [SMILES: COc1cc(NC(=O)NCc2cc(Cl)cc(C(F)(F)F)c2)cc2[nH]ccc12] |
| 504 | 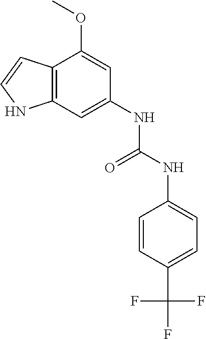 [SMILES: COc1cc(NC(=O)Nc2ccc(C(F)(F)F)cc2)cc2[nH]ccc12] |
| 505 | 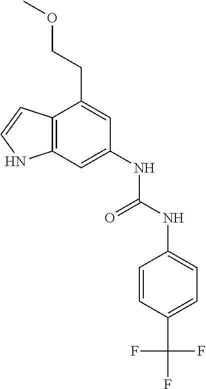 [SMILES: COCCc1cc(NC(=O)Nc2ccc(C(F)(F)F)cc2)cc2[nH]ccc12] |
| 506 | 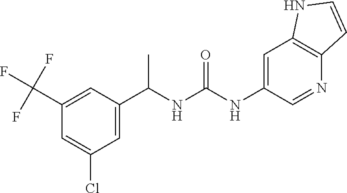 [SMILES: CC(NC(=O)Nc1cnc2cc[nH]c2c1)c1cc(Cl)cc(C(F)(F)F)c1] |
| 507 | 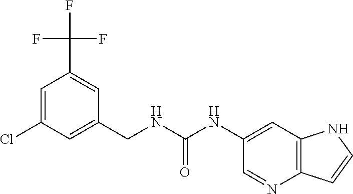 [SMILES: O=C(NCc1cc(Cl)cc(C(F)(F)F)c1)Nc1cnc2cc[nH]c2c1] |
| 508 | 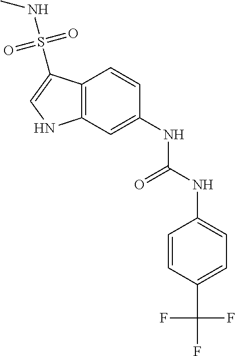 [SMILES: CNS(=O)(=O)c1c[nH]c2cc(NC(=O)Nc3ccc(C(F)(F)F)cc3)ccc12] |
| 509 | 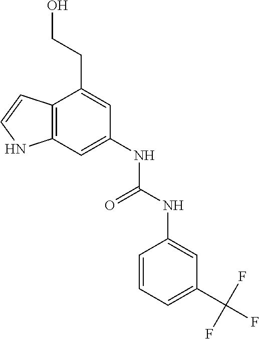 [SMILES: O=C(Nc1cccc(C(F)(F)F)c1)Nc1cc(CCO)c2cc[nH]c2c1] |
| 510 | 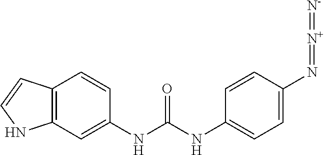 [SMILES: [N-]=[N+]=Nc1ccc(NC(=O)Nc2ccc3cc[nH]c3c2)cc1] |
| 511 | 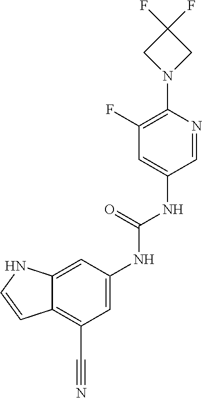 [SMILES: N#Cc1cc(NC(=O)Nc2cnc(N3CC(F)(F)C3)c(F)c2)cc2[nH]ccc12] |
| 512 | 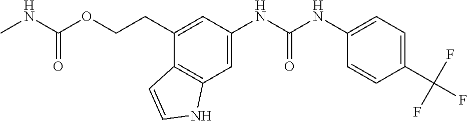 [SMILES: CNC(=O)OCCc1cc(NC(=O)Nc2ccc(C(F)(F)F)cc2)cc2[nH]ccc12] |
| 513 | 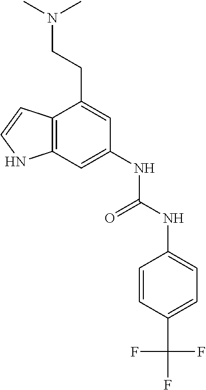 [SMILES: CN(C)CCc1cc(NC(=O)Nc2ccc(C(F)(F)F)cc2)cc2[nH]ccc12] |
| 514 | 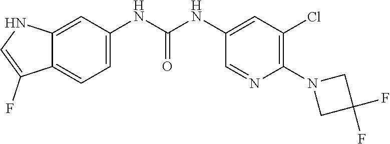 [SMILES: O=C(Nc1cnc(N2CC(F)(F)C2)c(Cl)c1)Nc1ccc2c(F)c[nH]c2c1] |
| 515 | 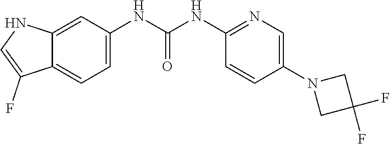 [SMILES: O=C(Nc1ccc2c(F)c[nH]c2c1)Nc1ccc(N2CC(F)(F)C2)cn1] |
| 516 | 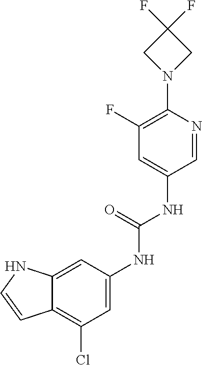 [SMILES: O=C(Nc1cnc(N2CC(F)(F)C2)c(F)c1)Nc1cc(Cl)c2cc[nH]c2c1] |
| 517 | 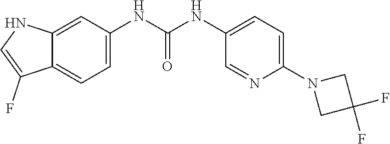 [SMILES: O=C(Nc1ccc(N2CC(F)(F)C2)nc1)Nc1ccc2c(F)c[nH]c2c1] |
| 518 | 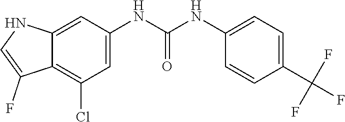 [SMILES: O=C(Nc1ccc(C(F)(F)F)cc1)Nc1cc(Cl)c2c(F)c[nH]c2c1] |
| 519 | 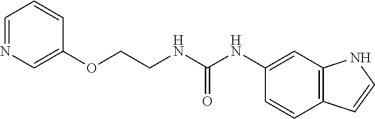 [SMILES: O=C(NCCOc1cccnc1)Nc1ccc2cc[nH]c2c1] |
| 520 | 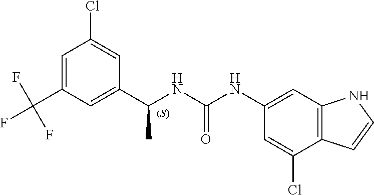 |
| 521 | 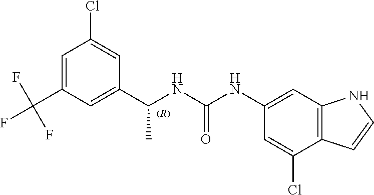 |
| 522 | 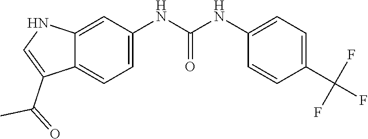 [SMILES: CC(=O)c1c[nH]c2cc(NC(=O)Nc3ccc(C(F)(F)F)cc3)ccc12] |
| 523 | 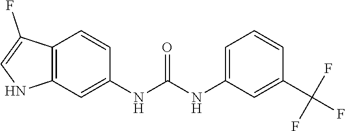 [SMILES: O=C(Nc1cccc(C(F)(F)F)c1)Nc1ccc2c(F)c[nH]c2c1] |
| 524 | 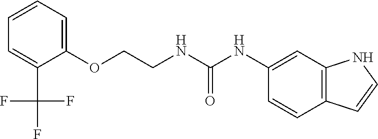 [SMILES: O=C(NCCOc1ccccc1C(F)(F)F)Nc1ccc2cc[nH]c2c1] |
| 525 | 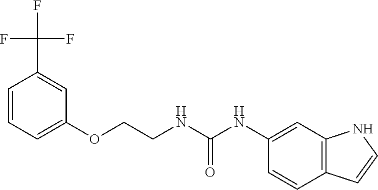 [SMILES: O=C(NCCOc1cccc(C(F)(F)F)c1)Nc1ccc2cc[nH]c2c1] |
| 526 | 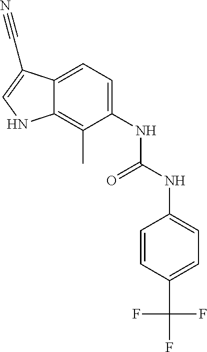 [SMILES: Cc1c(NC(=O)Nc2ccc(C(F)(F)F)cc2)ccc2c(C#N)c[nH]c12] |
| 527 | 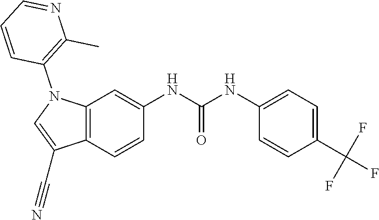 [SMILES: Cc1ncccc1-n1cc(C#N)c2ccc(NC(=O)Nc3ccc(C(F)(F)F)cc3)cc21] |
| 528 | 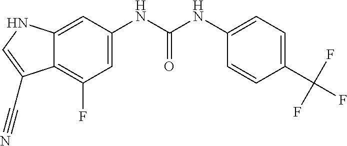 [SMILES: N#Cc1c[nH]c2cc(NC(=O)Nc3ccc(C(F)(F)F)cc3)cc(F)c12] |
| 529 | 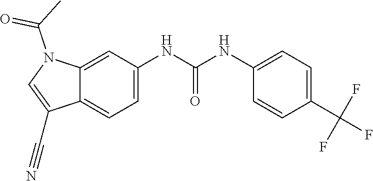 [SMILES: CC(=O)n1cc(C#N)c2ccc(NC(=O)Nc3ccc(C(F)(F)F)cc3)cc21] |
| 530 | 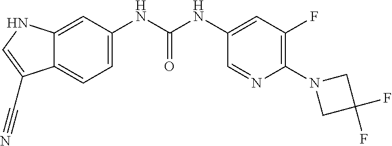 [SMILES: N#Cc1c[nH]c2cc(NC(=O)Nc3cnc(N4CC(F)(F)C4)c(F)c3)ccc12] |
| 531 | 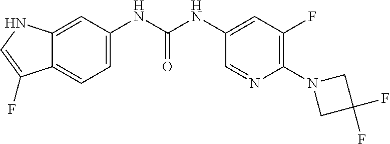 [SMILES: O=C(Nc1cnc(N2CC(F)(F)C2)c(F)c1)Nc1ccc2c(F)c[nH]c2c1] |
| 532 | 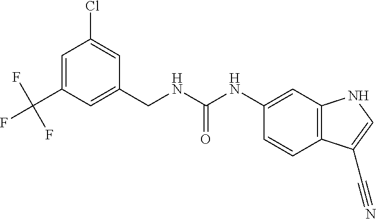 [SMILES: N#Cc1c[nH]c2cc(NC(=O)NCc3cc(Cl)cc(C(F)(F)F)c3)ccc12] |
| 533 | 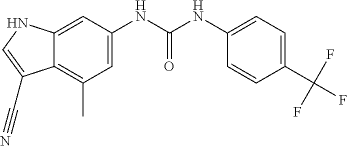 [SMILES: Cc1cc(NC(=O)Nc2ccc(C(F)(F)F)cc2)cc2[nH]cc(C#N)c12] |
| 534 | 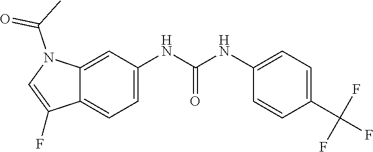 [SMILES: CC(=O)n1cc(F)c2ccc(NC(=O)Nc3ccc(C(F)(F)F)cc3)cc21] |
| 535 | 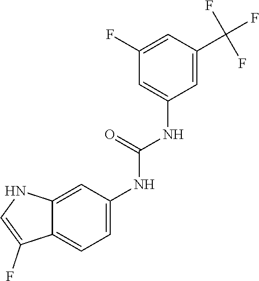 [SMILES: O=C(Nc1cc(F)cc(C(F)(F)F)c1)Nc1ccc2c(F)c[nH]c2c1] |
| 536 | 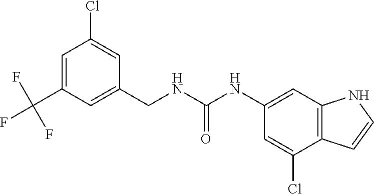 [SMILES: O=C(NCc1cc(Cl)cc(C(F)(F)F)c1)Nc1cc(Cl)c2cc[nH]c2c1] |
| 537 | 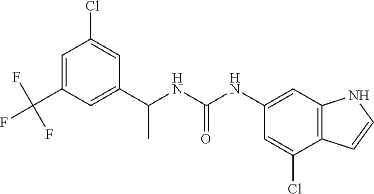 [SMILES: CC(NC(=O)Nc1cc(Cl)c2cc[nH]c2c1)c1cc(Cl)cc(C(F)(F)F)c1] |
| 538 | 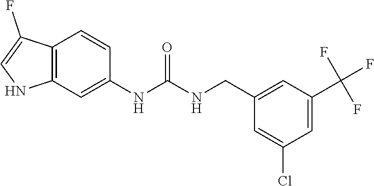 [SMILES: O=C(NCc1cc(Cl)cc(C(F)(F)F)c1)Nc1ccc2c(F)c[nH]c2c1] |
| 539 | 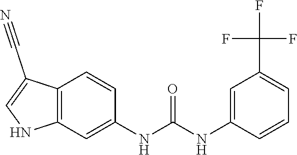 [SMILES: N#Cc1c[nH]c2cc(NC(=O)Nc3cccc(C(F)(F)F)c3)ccc12] |
| 540 | 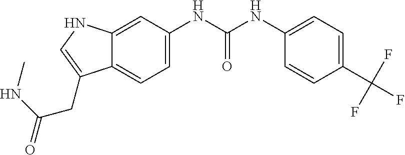 [SMILES: CNC(=O)Cc1c[nH]c2cc(NC(=O)Nc3ccc(C(F)(F)F)cc3)ccc12] |
| 541 | 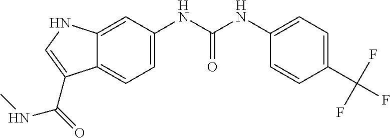 [SMILES: CNC(=O)c1c[nH]c2cc(NC(=O)Nc3ccc(C(F)(F)F)cc3)ccc12] |
| 542 | 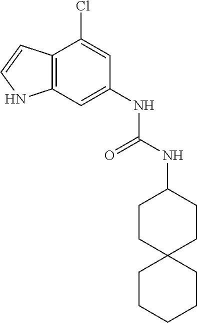 [SMILES: O=C(Nc1cc(Cl)c2cc[nH]c2c1)NC1CCC2(CCCCC2)CC1] |
| 543 | 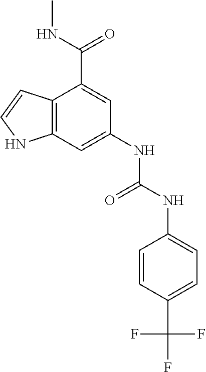 [SMILES: CNC(=O)c1cc(NC(=O)Nc2ccc(C(F)(F)F)cc2)cc2[nH]ccc12] |
| 544 | 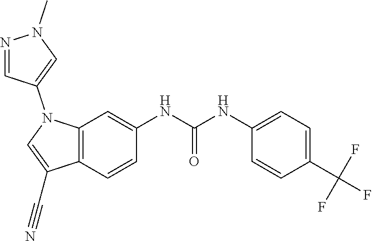 [SMILES: Cn1cc(-n2cc(C#N)c3ccc(NC(=O)Nc4ccc(C(F)(F)F)cc4)cc32)cn1] |
| 545 | 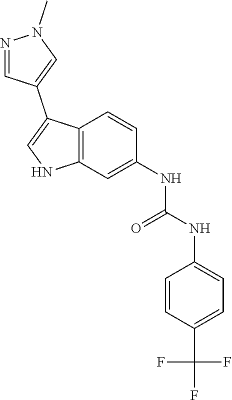 [SMILES: Cn1cc(-c2c[nH]c3cc(NC(=O)Nc4ccc(C(F)(F)F)cc4)ccc23)cn1] |
| 546 | 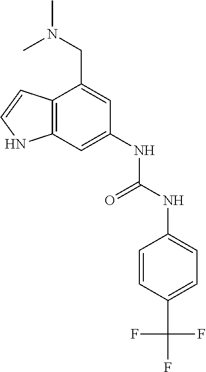 [SMILES: CN(C)Cc1cc(NC(=O)Nc2ccc(C(F)(F)F)cc2)cc2[nH]ccc12] |
| 547 | 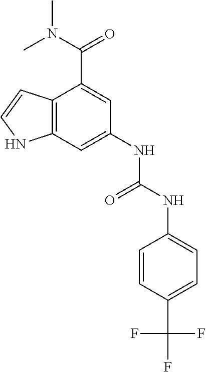 [SMILES: CN(C)C(=O)c1cc(NC(=O)Nc2ccc(C(F)(F)F)cc2)cc2[nH]ccc12] |
| 548 | 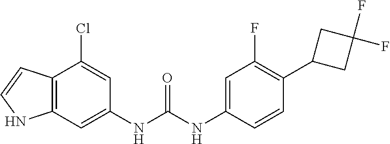 [SMILES: O=C(Nc1ccc(C2CC(F)(F)C2)c(F)c1)Nc1cc(Cl)c2cc[nH]c2c1] |
| 549 | 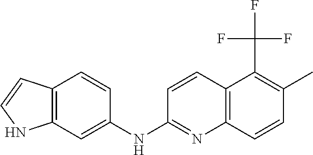 [SMILES: Cc1ccc2nc(Nc3ccc4cc[nH]c4c3)ccc2c1C(F)(F)F] |
| 550 | 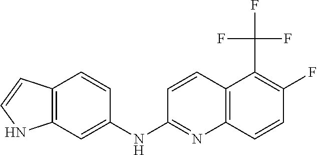 [SMILES: Fc1ccc2nc(Nc3ccc4cc[nH]c4c3)ccc2c1C(F)(F)F] |
| 551 | 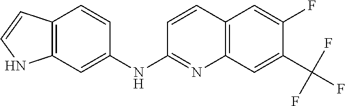 [SMILES: Fc1cc2ccc(Nc3ccc4cc[nH]c4c3)nc2cc1C(F)(F)F] |
| 552 | 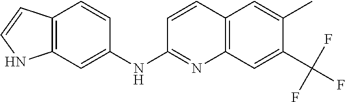 [SMILES: Cc1cc2ccc(Nc3ccc4cc[nH]c4c3)nc2cc1C(F)(F)F] |
| 553 | 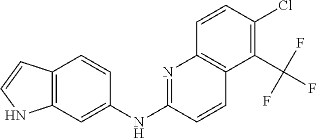 [SMILES: FC(F)(F)c1c(Cl)ccc2nc(Nc3ccc4cc[nH]c4c3)ccc12] |
| 554 | 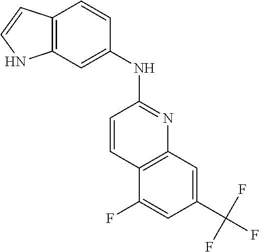 [SMILES: Fc1cc(C(F)(F)F)cc2nc(Nc3ccc4cc[nH]c4c3)ccc12] |
| 555 | 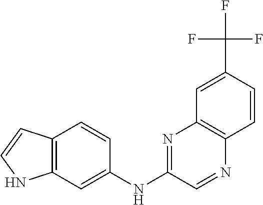 [SMILES: FC(F)(F)c1ccc2ncc(Nc3ccc4cc[nH]c4c3)nc2c1] |
| 556 | 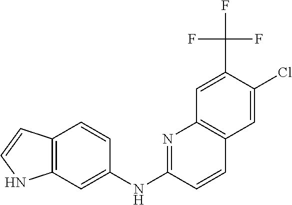 [SMILES: FC(F)(F)c1cc2nc(Nc3ccc4cc[nH]c4c3)ccc2cc1Cl] |
| 557 | 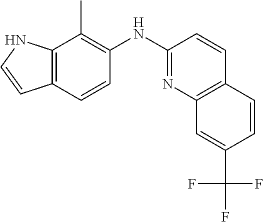 [SMILES: Cc1c(Nc2ccc3ccc(C(F)(F)F)cc3n2)ccc2cc[nH]c12] |
| 558 | 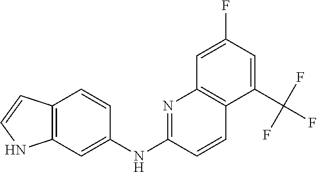 [SMILES: Fc1cc(C(F)(F)F)c2ccc(Nc3ccc4cc[nH]c4c3)nc2c1] |
| 559 | 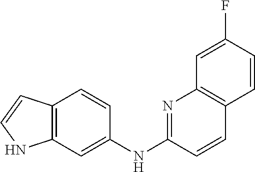 [SMILES: Fc1ccc2ccc(Nc3ccc4cc[nH]c4c3)nc2c1] |
| 560 | 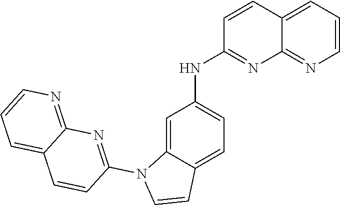 [SMILES: c1cnc2nc(Nc3ccc4ccn(-c5ccc6cccnc6n5)c4c3)ccc2c1] |
| 561 | 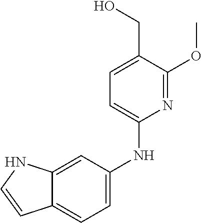 [SMILES: COc1nc(Nc2ccc3cc[nH]c3c2)ccc1CO] |
| 562 | 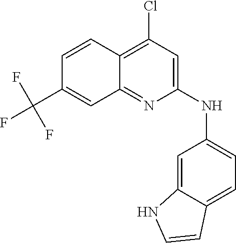 [SMILES: FC(F)(F)c1ccc2c(Cl)cc(Nc3ccc4cc[nH]c4c3)nc2c1] |
| 563 | 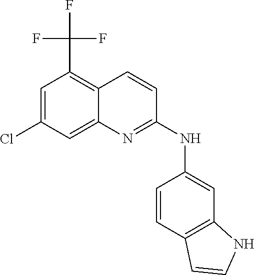 [SMILES: FC(F)(F)c1cc(Cl)cc2nc(Nc3ccc4cc[nH]c4c3)ccc12] |
| 564 | 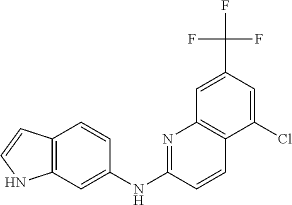 [SMILES: FC(F)(F)c1cc(Cl)c2ccc(Nc3ccc4cc[nH]c4c3)nc2c1] |
| 565 | 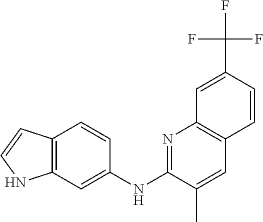 [SMILES: Cc1cc2ccc(C(F)(F)F)cc2nc1Nc1ccc2cc[nH]c2c1] |
| 566 | 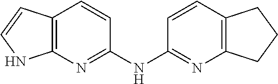 [SMILES: c1cc2ccc(Nc3ccc4c(n3)CCC4)nc2[nH]1] |
| 567 | 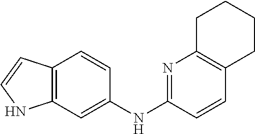 [SMILES: c1cc2ccc(Nc3ccc4c(n3)CCCC4)cc2[nH]1] |
| 568 | 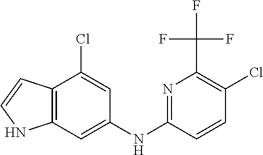 [SMILES: FC(F)(F)c1nc(Nc2cc(Cl)c3cc[nH]c3c2)ccc1Cl] |
| 569 | 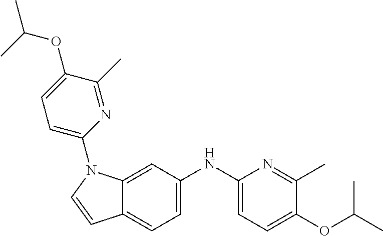 [SMILES: Cc1nc(Nc2ccc3ccn(-c4ccc(OC(C)C)c(C)n4)c3c2)ccc1OC(C)C] |
| 570 | 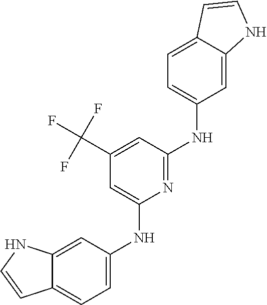 [SMILES: FC(F)(F)c1cc(Nc2ccc3cc[nH]c3c2)nc(Nc2ccc3cc[nH]c3c2)c1] |
| 571 | 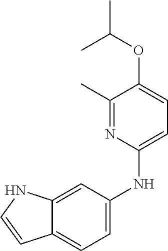 [SMILES: Cc1nc(Nc2ccc3cc[nH]c3c2)ccc1OC(C)C] |
| 572 | 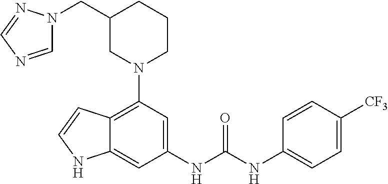 [SMILES: O=C(Nc1ccc(C(F)(F)F)cc1)Nc1cc(N2CCCC(Cn3cncn3)C2)c2cc[nH]c2c1] |
| 573 | 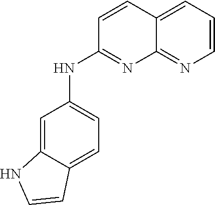 [SMILES: c1cnc2nc(Nc3ccc4cc[nH]c4c3)ccc2c1] |
| 574 | 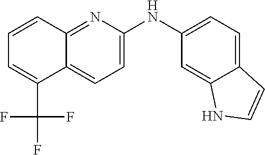 [SMILES: FC(F)(F)c1cccc2nc(Nc3ccc4cc[nH]c4c3)ccc12] |
| 575 | 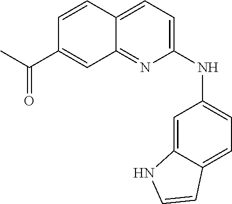 [SMILES: CC(=O)c1ccc2ccc(Nc3ccc4cc[nH]c4c3)nc2c1] |
| 576 | 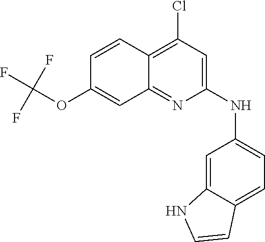 [SMILES: FC(F)(F)Oc1ccc2c(Cl)cc(Nc3ccc4cc[nH]c4c3)nc2c1] |
| 577 | 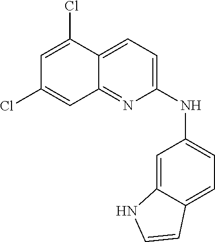 [SMILES: Clc1cc(Cl)c2ccc(Nc3ccc4cc[nH]c4c3)nc2c1] |
| 578 | 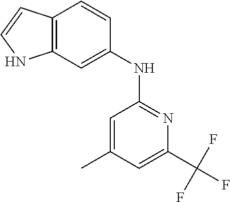 [SMILES: Cc1cc(Nc2ccc3cc[nH]c3c2)nc(C(F)(F)F)c1] |
| 579 | 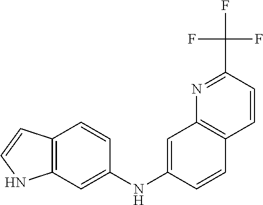 [SMILES: FC(F)(F)c1ccc2ccc(Nc3ccc4cc[nH]c4c3)cc2n1] |
| 580 | 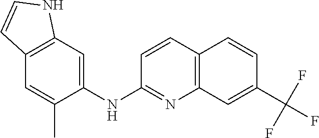 [SMILES: Cc1cc2cc[nH]c2cc1Nc1ccc2ccc(C(F)(F)F)cc2n1] |
| 581 | 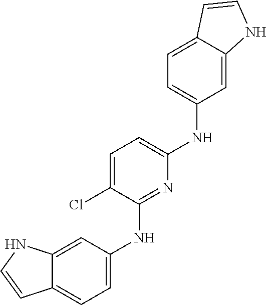 [SMILES: Clc1ccc(Nc2ccc3cc[nH]c3c2)nc1Nc1ccc2cc[nH]c2c1] |
| 582 | 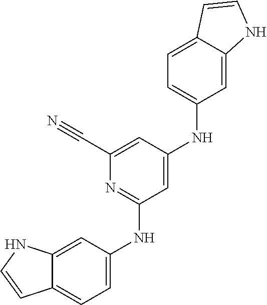 [SMILES: N#Cc1cc(Nc2ccc3cc[nH]c3c2)cc(Nc2ccc3cc[nH]c3c2)n1] |
| 583 | 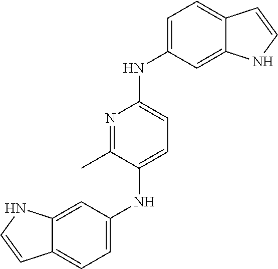 [SMILES: Cc1nc(Nc2ccc3cc[nH]c3c2)ccc1Nc1ccc2cc[nH]c2c1] |
| 584 | 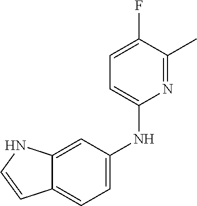 [SMILES: Cc1nc(Nc2ccc3cc[nH]c3c2)ccc1F] |
| 585 | 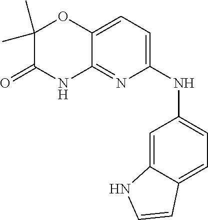 [SMILES: CC1(C)Oc2ccc(Nc3ccc4cc[nH]c4c3)nc2NC1=O] |
| 586 | 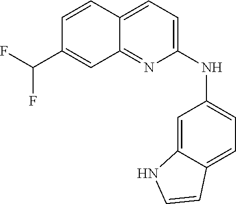 [SMILES: FC(F)c1ccc2ccc(Nc3ccc4cc[nH]c4c3)nc2c1] |
| 587 | 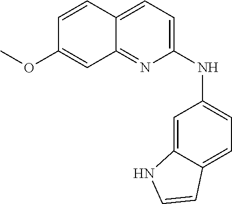 [SMILES: COc1ccc2ccc(Nc3ccc4cc[nH]c4c3)nc2c1] |
| 588 | 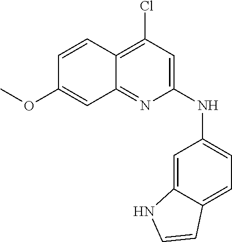 [SMILES: COc1ccc2c(Cl)cc(Nc3ccc4cc[nH]c4c3)nc2c1] |
| 589 | 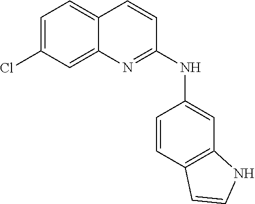 [SMILES: Clc1ccc2ccc(Nc3ccc4cc[nH]c4c3)nc2c1] |
| 590 | 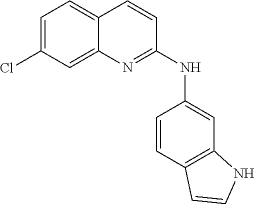 [SMILES: Clc1ccc2ccc(Nc3ccc4cc[nH]c4c3)nc2c1] |
| 591 | 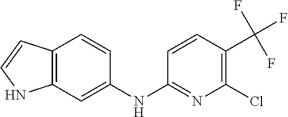 [SMILES: FC(F)(F)c1ccc(Nc2ccc3cc[nH]c3c2)nc1Cl] |
| 592 | 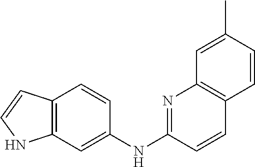 [SMILES: Cc1ccc2ccc(Nc3ccc4cc[nH]c4c3)nc2c1] |
| 593 | 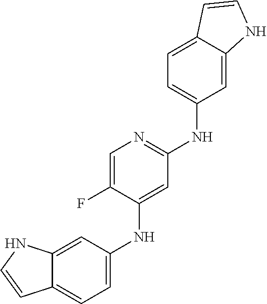 [SMILES: Fc1cnc(Nc2ccc3cc[nH]c3c2)cc1Nc1ccc2cc[nH]c2c1] |
| 594 | 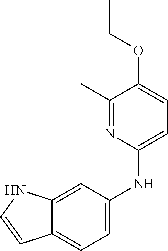 [SMILES: CCOc1ccc(Nc2ccc3cc[nH]c3c2)nc1C] |
| 595 | 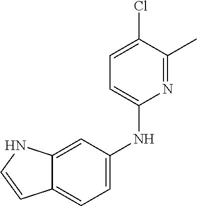 [SMILES: Cc1nc(Nc2ccc3cc[nH]c3c2)ccc1Cl] |
| 596 | 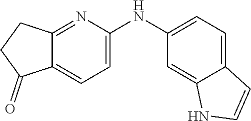 [SMILES: O=C1CCc2nc(Nc3ccc4cc[nH]c4c3)ccc21] |
| 597 | 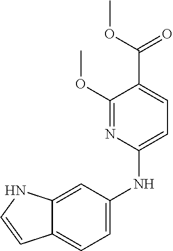 [SMILES: COC(=O)c1ccc(Nc2ccc3cc[nH]c3c2)nc1OC] |
| 598 | 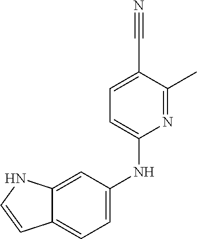 [SMILES: Cc1nc(Nc2ccc3cc[nH]c3c2)ccc1C#N] |
| 599 | 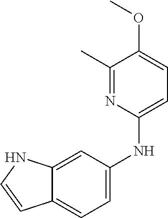 [SMILES: COc1ccc(Nc2ccc3cc[nH]c3c2)nc1C] |
| 600 | 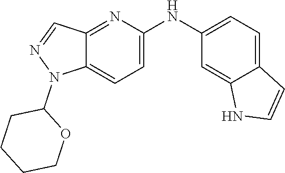 [SMILES: c1cc2ccc(Nc3ccc4c(cnn4C4CCCCO4)n3)cc2[nH]1] |
| 601 | 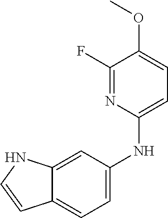 [SMILES: COc1ccc(Nc2ccc3cc[nH]c3c2)nc1F] |
| 602 | 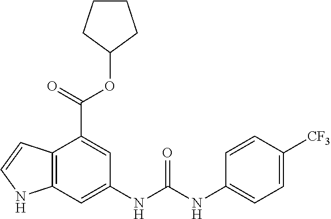 [SMILES: O=C(Nc1ccc(C(F)(F)F)cc1)Nc1cc(C(=O)OC2CCCC2)c2cc[nH]c2c1] |
| 603 | 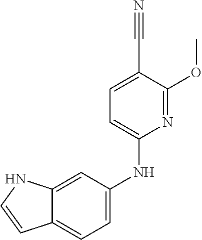 [SMILES: COc1nc(Nc2ccc3cc[nH]c3c2)ccc1C#N] |
| 604 | 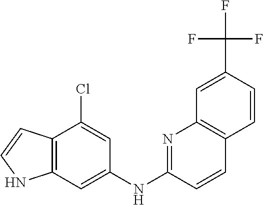 [SMILES: FC(F)(F)c1ccc2ccc(Nc3cc(Cl)c4cc[nH]c4c3)nc2c1] |
| 605 | 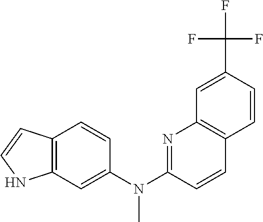 [SMILES: CN(c1ccc2cc[nH]c2c1)c1ccc2ccc(C(F)(F)F)cc2n1] |
| 606 | 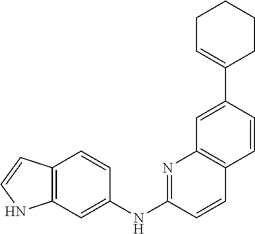 [SMILES: C1=C(c2ccc3ccc(Nc4ccc5cc[nH]c5c4)nc3c2)CCCC1] |
| 607 | 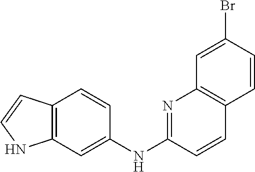 [SMILES: Brc1ccc2ccc(Nc3ccc4cc[nH]c4c3)nc2c1] |
| 608 | 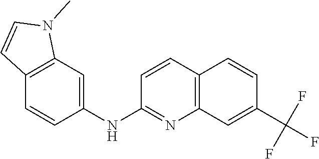 [SMILES: Cn1ccc2ccc(Nc3ccc4ccc(C(F)(F)F)cc4n3)cc21] |
| 609 | 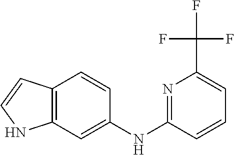 [SMILES: FC(F)(F)c1cccc(Nc2ccc3cc[nH]c3c2)n1] |
| 611 | 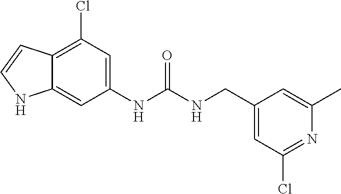 [SMILES: Cc1cc(CNC(=O)Nc2cc(Cl)c3cc[nH]c3c2)cc(Cl)n1] |
| 612 | 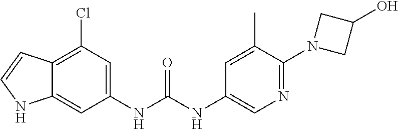 [SMILES: Cc1cc(NC(=O)Nc2cc(Cl)c3cc[nH]c3c2)cnc1N1CC(O)C1] |
| 613 | 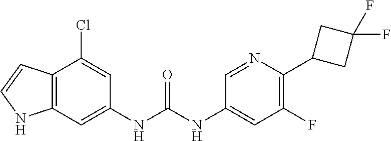 [SMILES: O=C(Nc1cnc(C2CC(F)(F)C2)c(F)c1)Nc1cc(Cl)c2cc[nH]c2c1] |
| 614 | 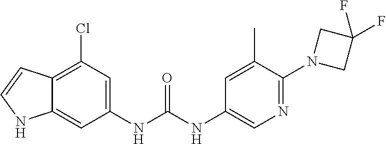 [SMILES: Cc1cc(NC(=O)Nc2cc(Cl)c3cc[nH]c3c2)cnc1N1CC(F)(F)C1] |
| 615 | 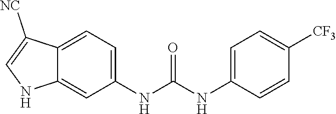 [SMILES: N#Cc1c[nH]c2cc(NC(=O)Nc3ccc(C(F)(F)F)cc3)ccc12] |
| 616 | 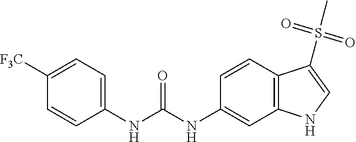 [SMILES: CS(=O)(=O)c1c[nH]c2cc(NC(=O)Nc3ccc(C(F)(F)F)cc3)ccc12] |
| 617 | 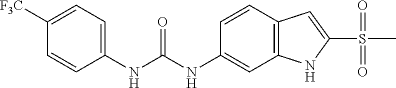 [SMILES: CS(=O)(=O)c1cc2ccc(NC(=O)Nc3ccc(C(F)(F)F)cc3)cc2[nH]1] |
| 618 | 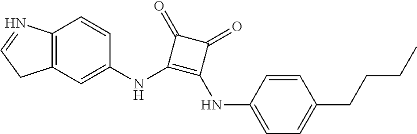 [SMILES: CCCCc1ccc(Nc2c(Nc3ccc4c(c3)CC=N4)c(=O)c2=O)cc1] |
| 619 | 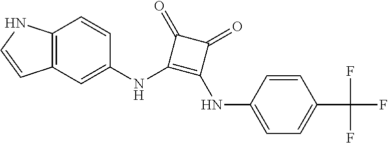 [SMILES: O=c1c(Nc2ccc(C(F)(F)F)cc2)c(Nc2ccc3[nH]ccc3c2)c1=O] |
| 620 | 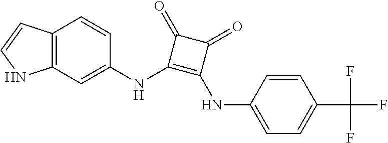 [SMILES: O=c1c(Nc2ccc(C(F)(F)F)cc2)c(Nc2ccc3cc[nH]c3c2)c1=O] |
| 621 | 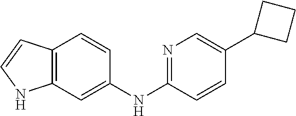 [SMILES: c1cc2ccc(Nc3ccc(C4CCC4)cn3)cc2[nH]1] |
| 622 | 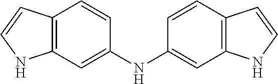 [SMILES: c1cc2ccc(Nc3ccc4cc[nH]c4c3)cc2[nH]1] |
| 623 | 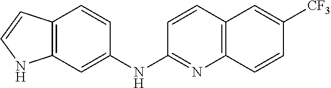 [SMILES: FC(F)(F)c1ccc2nc(Nc3ccc4cc[nH]c4c3)ccc2c1] |
| 624 | 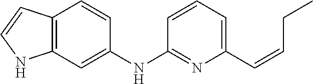 [SMILES: CC/C=C\c1cccc(Nc2ccc3cc[nH]c3c2)n1] |
| 625 | 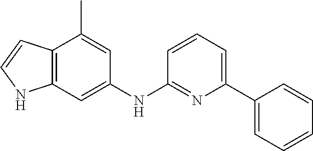 [SMILES: Cc1cc(Nc2cccc(-c3ccccc3)n2)cc2[nH]ccc12] |
| 626 | 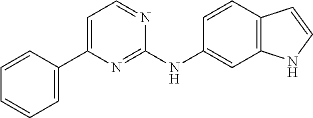 [SMILES: c1ccc(-c2ccnc(Nc3ccc4cc[nH]c4c3)n2)cc1] |
| 627 | 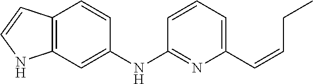 [SMILES: CC/C=C\c1cccc(Nc2ccc3cc[nH]c3c2)n1] |
| 628 | 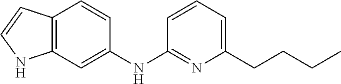 [SMILES: CCCCc1cccc(Nc2ccc3cc[nH]c3c2)n1] |
| 630 | 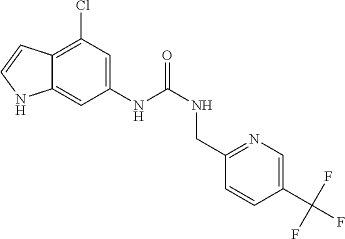 [SMILES: O=C(NCc1ccc(C(F)(F)F)cn1)Nc1cc(Cl)c2cc[nH]c2c1] |
| 631 | 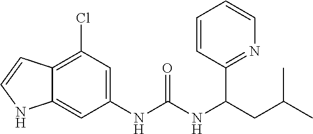 [SMILES: CC(C)CC(NC(=O)Nc1cc(Cl)c2cc[nH]c2c1)c1ccccn1] |
| 632 | 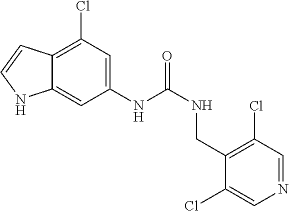 [SMILES: O=C(NCc1c(Cl)cncc1Cl)Nc1cc(Cl)c2cc[nH]c2c1] |
| 633 | 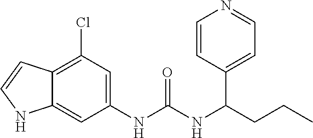 [SMILES: CCCC(NC(=O)Nc1cc(Cl)c2cc[nH]c2c1)c1ccncc1] |
| 634 | 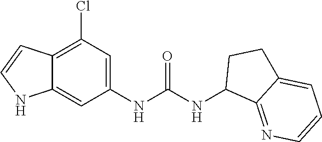 [SMILES: O=C(Nc1cc(Cl)c2cc[nH]c2c1)NC1CCc2cccnc21] |
| 635 | 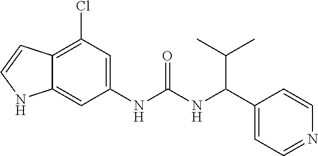 [SMILES: CC(C)C(NC(=O)Nc1cc(Cl)c2cc[nH]c2c1)c1ccncc1] |
| 636 | 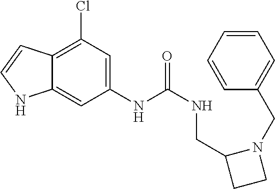 [SMILES: O=C(NCC1CCN1Cc1ccccc1)Nc1cc(Cl)c2cc[nH]c2c1] |
| 637 | 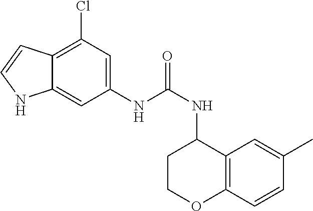 [SMILES: Cc1ccc2c(c1)C(NC(=O)Nc1cc(Cl)c3cc[nH]c3c1)CCO2] |
| 638 | 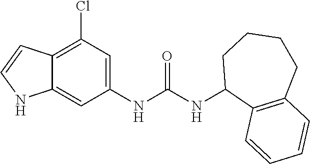 [SMILES: O=C(Nc1cc(Cl)c2cc[nH]c2c1)NC1CCCCc2ccccc21] |
| 639 | 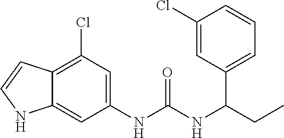 [SMILES: CCC(NC(=O)Nc1cc(Cl)c2cc[nH]c2c1)c1cccc(Cl)c1] |
| 640 | 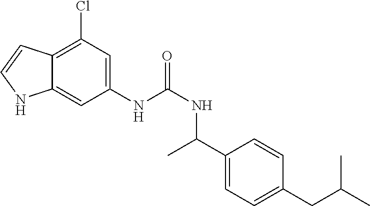 [SMILES: CC(C)Cc1ccc(C(C)NC(=O)Nc2cc(Cl)c3cc[nH]c3c2)cc1] |
| 641 | 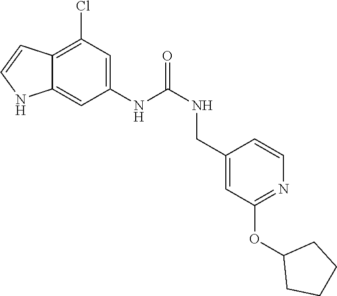 [SMILES: O=C(NCc1ccnc(OC2CCCC2)c1)Nc1cc(Cl)c2cc[nH]c2c1] |
| 642 | 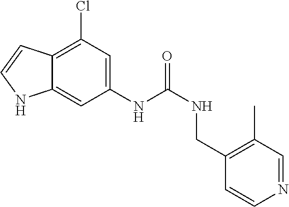 [SMILES: Cc1cnccc1CNC(=O)Nc1cc(Cl)c2cc[nH]c2c1] |
| 643 | 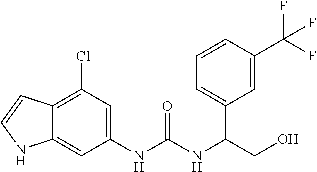 [SMILES: O=C(Nc1cc(Cl)c2cc[nH]c2c1)NC(CO)c1cccc(C(F)(F)F)c1] |
| 644 | 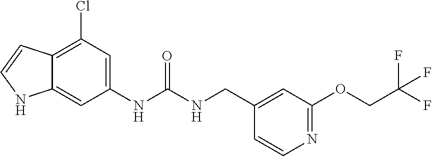 [SMILES: O=C(NCc1ccnc(OCC(F)(F)F)c1)Nc1cc(Cl)c2cc[nH]c2c1] |
| 645 | 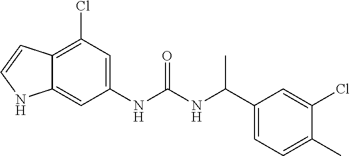 [SMILES: Cc1ccc(C(C)NC(=O)Nc2cc(Cl)c3cc[nH]c3c2)cc1Cl] |
| 646 | 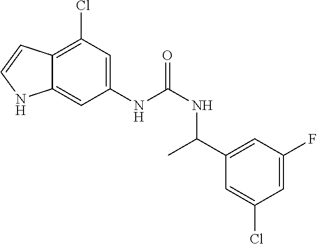 [SMILES: CC(NC(=O)Nc1cc(Cl)c2cc[nH]c2c1)c1cc(F)cc(Cl)c1] |
| 647 | 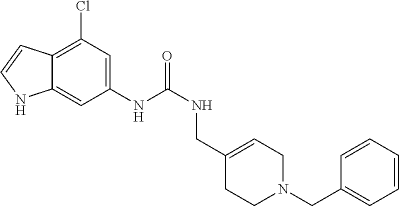 [SMILES: O=C(NCC1=CCN(Cc2ccccc2)CC1)Nc1cc(Cl)c2cc[nH]c2c1] |
| 648 | 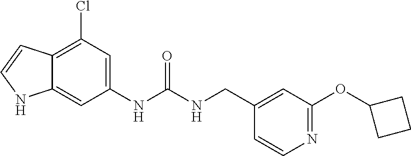 [SMILES: O=C(NCc1ccnc(OC2CCC2)c1)Nc1cc(Cl)c2cc[nH]c2c1] |
| 649 | 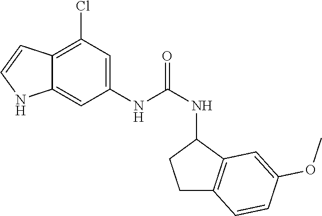 [SMILES: COc1ccc2c(c1)C(NC(=O)Nc1cc(Cl)c3cc[nH]c3c1)CC2] |
| 650 | 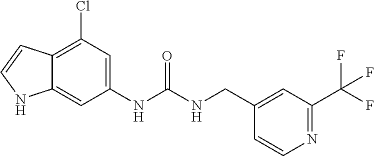 [SMILES: O=C(NCc1ccnc(C(F)(F)F)c1)Nc1cc(Cl)c2cc[nH]c2c1] |
| 651 | 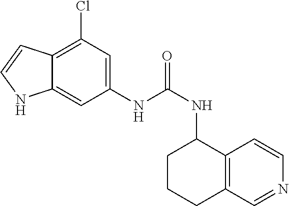 [SMILES: O=C(Nc1cc(Cl)c2cc[nH]c2c1)NC1CCCc2cnccc21] |
| 652 | 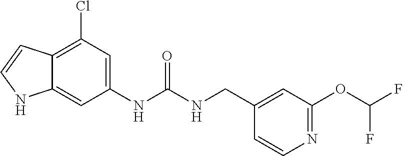 [SMILES: O=C(NCc1ccnc(OC(F)F)c1)Nc1cc(Cl)c2cc[nH]c2c1] |
| 653 | 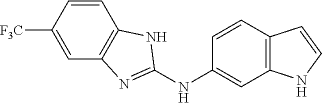 [SMILES: FC(F)(F)c1ccc2[nH]c(Nc3ccc4cc[nH]c4c3)nc2c1] |
| 654 | 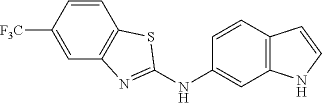 [SMILES: FC(F)(F)c1ccc2sc(Nc3ccc4cc[nH]c4c3)nc2c1] |
| 655 | 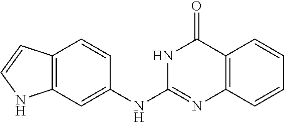 [SMILES: O=c1[nH]c(Nc2ccc3cc[nH]c3c2)nc2ccccc12] |
| 656 | 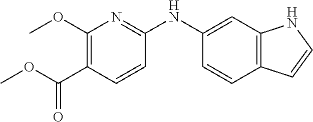 [SMILES: COC(=O)c1ccc(Nc2ccc3cc[nH]c3c2)nc1OC] |

Pharmaceutical Compositions and Administration

General

In some embodiments, a chemical entity (e.g., a compound that inhibits (e.g., antagonizes) STING, or a pharmaceutically acceptable salt, and/or hydrate, and/or cocrystal, and/or drug combination thereof) is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In certain embodiments, a preferred route of administration is parenteral (e.g., intratumoral).

Compositions can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle so which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Intratumoral injections are discussed, e.g., in Lammers, et al., “Effect of Intratumoral Injection on the Biodistribution and the Therapeutic Potential of HPMA Copolymer-Based Drug Delivery Systems” Neoplasia. 2006, 10, 788-795.

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In other embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

Dosages

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In some embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Methods of Treatment

In some embodiments, methods for treating a subject having condition, disease or disorder in which increased (e.g., excessive) STING activity (e.g., , e.g., STING signaling) contributes to the pathology and/or symptoms and/or progression of the condition, disease or disorder (e.g., immune disorders, cancer) are provided.

Indications

In some embodiments, the condition, disease or disorder is cancer. Non-limiting examples of cancer include melanoma, carcinoma, lymphoma, blastoma, sarcoma, and leukemia or lymphoid malignancies. More particular examples of such cancers include breast cancer, colon cancer, rectal cancer, colorectal cancer, kidney or renal cancer, clear cell cancer lung cancer including small-cell lung cancer, non-small cell lung cancer, adenocarcinoma of the lung and squamous carcinoma of the lung, squamous cell cancer (e.g. epithelial squamous cell cancer), cervical cancer, ovarian cancer, prostate cancer, prostatic neoplasms, liver cancer, bladder cancer, cancer of the peritoneum, hepatocellular cancer, gastric or stomach cancer including gastrointestinal cancer, gastrointestinal stromal tumor, pancreatic cancer, head and neck cancer, glioblastoma, retinoblastoma, astrocytoma, thecomas, arrhenoblastomas, hepatoma, hematologic malignancies including non-Hodgkins lymphoma (NHL), multiple myeloma, myelodysplasia disorders, myeloproliferative disorders, chronic myelogenous leukemia, and acute hematologic malignancies, endometrial or uterine carcinoma, endometriosis, endometrial stromal sarcoma, fibrosarcomas, choriocarcinoma, salivary gland carcinoma, vulval cancer, thyroid cancer, esophageal carcinomas, hepatic carcinoma, anal carcinoma, penile carcinoma, nasopharyngeal carcinoma, laryngeal carcinomas, Kaposi's sarcoma, mast cell sarcoma, ovarian sarcoma, uterine sarcoma, melanoma, malignant mesothelioma, skin carcinomas, Schwannoma, oligodendroglioma, neuroblastomas, neuroectodermal tumor, rhabdomyosarcoma, osteogenic sarcoma, leiomyosarcomas, Ewing Sarcoma, peripheral primitive neuroectodermal tumor, urinary tract carcinomas, thyroid carcinomas, Wilm's tumor, as well as abnormal vascular proliferation associated with phakomatoses, edema (such as that associated with brain tumors), and Meigs' syndrome. In some cases, the cancer is melanoma.

In some embodiments, the condition, disease or disorder is a neurological disorder, which includes disorders that involve the central nervous system (brain, brainstem and cerebellum), the peripheral nervous system (including cranial nerves), and the autonomic nervous system (parts of which are located in both central and peripheral nervous system). Non-limiting examples of cancer include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; milti-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffian disease; West syndrome; whiplash; Williams syndrome; Wildon's disease; amyotrophe lateral sclerosis and Zellweger syndrome.

In some embodiments, the condition, disease or disorder is STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis. In certain embodiments, the condition, disease or disorder is an autoimmune disease (e.g., a cytosolic DNA-triggered autoinflammatory disease). Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In some embodiments, modulation of the immune system by STING provides for the treatment of diseases, including diseases caused by foreign agents. Exemplary infections by foreign agents which may be treated and/or prevented by the method of the present invention include an infection by a bacterium (e.g., a Gram-positive or Gram-negative bacterium), an infection by a fungus, an infection by a parasite, and an infection by a virus. In one embodiment of the present invention, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant enterococcus), or sepsis. In another embodiment, the infection is a fungal infection (e.g. infection by a mould, a yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus)).

In some embodiments, the condition, disease or disorder is hepatitis B (see, e.g., WO 2015/061294).

In some embodiments, the condition, disease or disorder is selected from cardiovascular diseases (including e.g., myocardial infarction).

In some embodiments, the condition, disease or disorder is age-related macular degeneration.

In some embodiments, the condition, disease or disorder is mucositis, also known as stomatitits, which can occur as a result of chemotherapy or radiation therapy, either alone or in combination as well as damage caused by exposure to radiation outside of the context of radiation therapy.

In some embodiments, the condition, disease or disorder is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition, disease or disorder is selected from the group consisting of a cancer, a neurological disorder, an autoimmune disease, hepatitis B, uvetitis, a cardiovascular disease, age-related macular degeneration, and mucositis.

Still other examples can include those indications discussed herein and below in contemplated combination therapy regimens.

Combination Therapy

This disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In certain embodiments, the methods described herein can further include administering one or more additional cancer therapies.

The one or more additional cancer therapies can include, without limitation, surgery, radiotherapy, chemotherapy, toxin therapy, immunotherapy, cryotherapy, cancer vaccines (e.g., HPV vaccine, hepatitis B vaccine, Oncophage, Provenge) and gene therapy, as well as combinations thereof. Immunotherapy, including, without limitation, adoptive cell therapy, the derivation of stem cells and/or dendritic cells, blood transfusions, lavages, and/or other treatments, including, without limitation, freezing a tumor.

In some embodiments, the one or more additional cancer therapies is chemotherapy, which can include administering one or more additional chemotherapeutic agents.

In certain embodiments, the additional chemotherapeutic agent is an immunomodulatory moiety, e.g., an immune checkpoint inhibitor. In certain of these embodiments, the immune checkpoint inhibitor targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9—TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain embodiments, the additional chemotherapeutic agent is an alkylating agent. Alkylating agents are so named because of their ability to alkylate many nucleophilic functional groups under conditions present in cells, including, but not limited to cancer cells. In a further embodiment, an alkylating agent includes, but is not limited to, Cisplatin, carboplatin, mechlorethamine, cyclophosphamide, chlorambucil, ifosfamide and/or oxaliplatin. In an embodiment, alkylating agents can function by impairing cell function by forming covalent bonds with the amino, carboxyl, sulfhydryl, and phosphate groups in biologically important molecules or they can work by modifying a cell's DNA. In a further embodiment an alkylating agent is a synthetic, semisynthetic or derivative.

In certain embodiments, the additional chemotherapeutic agent is an anti-metabolite. Anti-metabolites masquerade as purines or pyrimidines, the building-blocks of DNA and in general, prevent these substances from becoming incorporated in to DNA during the “S” phase (of the cell cycle), stopping normal development and division. Anti-metabolites can also affect RNA synthesis. In an embodiment, an antimetabolite includes, but is not limited to azathioprine and/or mercaptopurine. In a further embodiment an anti-metabolite is a synthetic, semisynthetic or derivative.

In certain embodiments, the additional chemotherapeutic agent is a plant alkaloid and/or terpenoid. These alkaloids are derived from plants and block cell division by, in general, preventing microtubule function. In an embodiment, a plant alkaloid and/or terpenoid is a vinca alkaloid, a podophyllotoxin and/or a taxane. Vinca alkaloids, in general, bind to specific sites on tubulin, inhibiting the assembly of tubulin into microtubules, generally during the M phase of the cell cycle. In an embodiment, a vinca alkaloid is derived, without limitation, from the Madagascar periwinkle, Catharanthus roseus (formerly known as Vinca rosea). In an embodiment, a vinca alkaloid includes, without limitation, Vincristine, Vinblastine, Vinorelbine and/or Vindesine. In an embodiment, a taxane includes, but is not limited, to Taxol, Paclitaxel and/or Docetaxel. In a further embodiment a plant alkaloid or terpernoid is a synthetic, semisynthetic or derivative. In a further embodiment, a podophyllotoxin is, without limitation, an etoposide and/or teniposide. In an embodiment, a taxane is, without limitation, docetaxel and/or ortataxel. [021] In an embodiment, a cancer therapeutic is a topoisomerase. Topoisomerases are essential enzymes that maintain the topology of DNA. Inhibition of type I or type II topoisomerases interferes with both transcription and replication of DNA by upsetting proper DNA supercoiling. In a further embodiment, a topoisomerase is, without limitation, a type I topoisomerase inhibitor or a type II topoisomerase inhibitor. In an embodiment a type I topoisomerase inhibitor is, without limitation, a camptothecin. In another embodiment, a camptothecin is, without limitation, exatecan, irinotecan, lurtotecan, topotecan, BNP 1350, CKD 602, DB 67 (AR67) and/or ST 1481. In an embodiment, a type II topoisomerase inhibitor is, without limitation, epipodophyllotoxin. In a further embodiment an epipodophyllotoxin is, without limitation, an amsacrine, etoposid, etoposide phosphate and/or teniposide. In a further embodiment a topoisomerase is a synthetic, semisynthetic or derivative, including those found in nature such as, without limitation, epipodophyllotoxins, substances naturally occurring in the root of American Mayapple (Podophyllum peltatum).

In certain embodiments, the additional chemotherapeutic agent is a stilbenoid. In a further embodiment, a stilbenoid includes, but is not limited to, Resveratrol, Piceatannol, Pinosylvin, Pterostilbene, Alpha-Viniferin, Ampelopsin A, Ampelopsin E, Diptoindonesin C, Diptoindonesin F, Epsilon-Vinferin, Flexuosol A, Gnetin H, Hemsleyanol D, Hopeaphenol, Trans-Diptoindonesin B, Astringin, Piceid and Diptoindonesin A. In a further embodiment a stilbenoid is a synthetic, semisynthetic or derivative.

In certain embodiments, the additional chemotherapeutic agent is a cytotoxic antibiotic. In an embodiment, a cytotoxic antibiotic is, without limitation, an actinomycin, an anthracenedione, an anthracycline, thalidomide, dichloroacetic acid, nicotinic acid, 2-deoxyglucose and/or chlofazimine. In an embodiment, an actinomycin is, without limitation, actinomycin D, bacitracin, colistin (polymyxin E) and/or polymyxin B. In another embodiment, an antracenedione is, without limitation, mitoxantrone and/or pixantrone. In a further embodiment, an anthracycline is, without limitation, bleomycin, doxorubicin (Adriamycin), daunorubicin (daunomycin), epirubicin, idarubicin, mitomycin, plicamycin and/or valrubicin. In a further embodiment a cytotoxic antibiotic is a synthetic, semisynthetic or derivative.

In certain embodiments, the additional chemotherapeutic agent is selected from endostatin, angiogenin, angiostatin, chemokines, angioarrestin, angiostatin (plasminogen fragment), basement-membrane collagen-derived anti-angiogenic factors (tumstatin, canstatin, or arrestin), anti-angiogenic antithrombin III, signal transduction inhibitors, cartilage-derived inhibitor (CDI), CD59 complement fragment, fibronectin fragment, gro-beta, heparinases, heparin hexasaccharide fragment, human chorionic gonadotropin (hCG), interferon alpha/beta/gamma, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), various retinoids, tetrahydrocortisol-S, thrombospondin-1 (TSP-1), transforming growth factor-beta (TGF-β), vasculostatin, vasostatin (calreticulin fragment) and the like.

In certain embodiments, the additional chemotherapeutic agent is selected from abiraterone acetate, altretamine, anhydrovinblastine, auristatin, bexarotene, bicalutamide, BMS 184476, 2,3,4,5,6-pentafluoro-N-(3-fluoro-4-methoxyphenyl)benzene sulfonamide, bleomycin, N,N-dimethyl-L-valyl-L-valyl-N-methyl-L-valyl-L-proly-1-Lproline-t-butylamide, cachectin, cemadotin, chlorambucil, cyclophosphamide, 3′,4′-didehydro-4′-deoxy-8′-norvin-caleukoblastine, docetaxol, doxetaxel, cyclophosphamide, carboplatin, carmustine, cisplatin, cryptophycin, cyclophosphamide, cytarabine, dacarbazine (DTIC), dactinomycin, daunorubicin, decitabine dolastatin, doxorubicin (adriamycin), etoposide, 5-fluorouracil, finasteride, flutamide, hydroxyurea and hydroxyureataxanes, ifosfamide, liarozole, lonidamine, lomustine (CCNU), MDV3100, mechlorethamine (nitrogen mustard), melphalan, mivobulin isethionate, rhizoxin, sertenef, streptozocin, mitomycin, methotrexate, taxanes, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In certain embodiments, the additional chemotherapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In still other embodiments, the additional chemotherapeutic agent can be selected from those delineated in U.S. Pat. No. 7,927,613, which is incorporated herein by reference in its entirety.

In some embodiments, the additional therapeutic agent and/or regimen are those that can be used for treating other STING-associated conditions, e.g., type I interferonopathies (e.g., STING-associated vasculopathy with onset in infancy (SAVI)), Aicardi-Goutières Syndrome (AGS), genetic forms of lupus, and inflammation-associated disorders such as systemic lupus erythematosus, and rheumatoid arthritis and the like.

Non-limiting examples of additional therapeutic agents and/or regimens for treating rheumatoid arthritis include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

Non-limiting examples of additional therapeutic agents and/or regimens for treating lupus include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, Gengraf®), and mycophenolate mofetil) baricitinb, iguratimod, filogotinib, GS-9876, rapamycin, and PF-06650833), and biologics (e.g., belimumab (Benlysta®), anifrolumab, prezalumab, MEDIO700, obinutuzumab, vobarilizumab, lulizumab, atacicept, PF-06823859, and lupizor, rituximab, BT063, BI655064, B1B059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, Gengraf®), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, B1B059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

Non-limiting examples of additional therapeutic agents and/or regimens for treating STING-associated vasculopathy with onset in infancy (SAVI) include JAK inhibitors (e.g., tofacitinib, ruxolitinib, filgotinib, and baricitinib).

Non-limiting examples of additional therapeutic agents and/or regimens for treating Aicardi-Goutières Syndrome (AGS) include physiotherapy, treatment for respiratory complications, anticonvulsant therapies for seizures, tube-feeding, nucleoside reverse transcriptase inhibitors (e.g., emtricitabine (e.g., Emtriva®), tenofovir (e.g., Viread®), emtricitabine/tenofovir (e.g., Truvada®), zidovudine, lamivudine, and abacavir), and JAK inhibitors (e.g., tofacitinib, ruxolitinib, filgotinib, and baricitinib).

Non-limiting examples of additional therapeutic agents and/or regimens for treating IBDs include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

Non-limiting examples of additional therapeutic agents and/or regimens for treating irritable bowel syndrome include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

Non-limiting examples of additional therapeutic agents and/or regimens for treating scleroderma include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, Gengraf®), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

Non-limiting examples of additional therapeutic agents and/or regimens for treating Crohn's Disease (CD) include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

Non-limiting examples of additional therapeutic agents and/or regimens for treating UC include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

Non-limiting examples of additional therapeutic agents and/or regimens for treating autoimmune colitis include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating iatrogenic autoimmune colitis include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis induced by one or more chemotherapeutics agents include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

Non-limiting examples of additional therapeutic agents and/or regimens for treating colitis induced by treatment with adoptive cell therapy include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

Non-limiting examples of additional therapeutic agents and/or regimens for treating colitis associated with one or more alloimmune diseases include corticosteroids (e.g., budesonide, prednisone, prednisolone, beclometasone dipropionate), sulfasalazine, and eicopentaenoic acid.

Non-limiting examples of additional therapeutic agents and/or regimens for treating radaiation enteritis include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

Non-limiting examples of additional therapeutic agents and/or regimens for treating collagenous colitis include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

Non-limiting examples of additional therapeutic agents and/or regimens for treating lyphocytic colitis include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

Non-limiting examples of additional therapeutic agents and/or regimens for treating microscopic colitis include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

Non-limiting examples of additional therapeutic agents and/or regimens for treating alloimmune disease include intrauterine platelet transfusions, intravenous immunoglobin, maternal steroids, abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating multiple sclerosis (MS) include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

Non-limiting examples of additional therapeutic agents and/or regimens for treating graft-vs-host disease include abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

Non-limiting examples of additional therapeutic agents and/or regimens for treating acute graft-vs-host disease include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

Non-limiting examples of additional therapeutic agents and/or regimens for treating chronic graft vs. host disease include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

Non-limiting examples of additional therapeutic agents and/or regimens for treating celiac disease include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

Non-limiting examples of additional therapeutic agents and/or regimens for treating psoriasis include topical corticosteroids, topical crisaborole/AN2728, topical SNA-120, topical SANO21, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-Scalp® and Dritho-crème®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, Gengraf®), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

Non-limiting examples of additional therapeutic agents and/or regimens for treating cutaneous T-cell lymphoma include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

Non-limiting examples of additional therapeutic agents and/or regimens for treating uveitis include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengraf®), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

Non-limiting examples of additional therapeutic agents and/or regimens for treating mucositis include AG013, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising vaccinium myrtillus extract, macleaya cordata alkaloids and echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

Patient Selection

In some embodiments, the methods described herein further include the step of identifying a subject (e.g., a patient) in need of such treatment (e.g., by way of biopsy, endoscopy, or other conventional method known in the art). In certain embodiments, the STING protein can serve as a biomarker for certain types of cancer, e.g., colon cancer and prostate cancer. In other embodiments, identifying a subject can include assaying the patient's tumor microenvironment for the absence of T-cells and/or presence of exhausted T-cells, e.g., patients having one or more cold tumors. Such patients can include those that are resistant to treatment with checkpoint inhibitors. In certain embodiments, such patients can be treated with a chemical entity herein, e.g., to recruit T-cells into the tumor, and in some cases, further treated with one or more checkpoint inhibitors, e.g., once the T-cells become exhausted.

In some embodiments, the chemical entities, methods, and compositions described herein can be administered to certain treatment-resistant patient populations (e.g., patients resistant to checkpoint inhibitors; e.g., patients having one or more cold tumors, e.g., tumors lacking T-cells or exhausted T-cells).

Compound Preparation

As can be appreciated by the skilled artisan, methods of synthesizing the compounds of the formulae herein will be evident to those of ordinary skill in the art. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

To further illustrate the foregoing, the following non-limiting, exemplary synthetic schemes are included. Variations of these examples within the scope of the claims are within the purview of one skilled in the art and are considered to fall within the scope of the invention as described, and claimed herein. The reader will recognize that the skilled artisan, provided with the present disclosure, and skill in the art is able to prepare and use the invention without exhaustive examples.

### EXAMPLES

Compounds 101 through 152 are synthesized using methods known to one of ordinary skill in the art.

As a non-limiting example, Compound 101 can be prepared as shown below

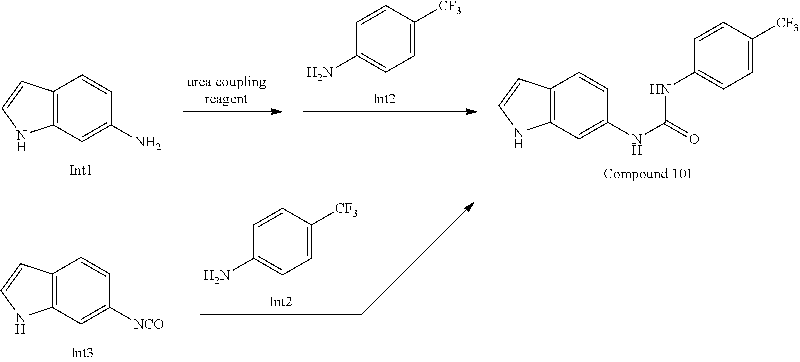

Int1 is treated with a urea coupling agent under basic conditions. Reaction of the resulting intermediate with Int2 affords compound 101.

Alternatively, isocyanate Int3 is treated with Int2 to afford compound 101.

Compound No. 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, and 154 in Table C1 can be prepared using methods similar to above:

### Preparative Examples

### Abbreviation of Chemical Terms

ACN = acetonitrile
AcOH = Acetic acid
Brettphos Pd G3 = Methanesulfonato(2-dicyclohexylphosphino-3,6-
dimethoxy-2′,4′,6′-tri-i-propyl-1,1′-biphenyl)(2′-amino-1,1′-biphenyl-
2-yl)palladium(II)
Bu = butyl
CSI = Chlorosulfonyl isocyanate
DAST = diethylaminosulphur trifluoride
DCC = N,N′-dicyclohexylcarbodiimide
DCE = 1,2-dichloroethane
DCM = dichloromethane
DIAD = Diisopropylazodicarboxylate
DIEA = N,N-diisopropylethylamine
DIEA = N,N-diisopropylethylamine
DMAP = 4-dimethylaminopyridine
DMF = Dimethyl formamide
DMF = N,N-dimethylformamide
DMF-DMA = N,N-dimethylformamide dimethyl acetal
DMSO = dimethyl sulfoxide
DPPA = diphenyl azidophosphate
Dppf = bis(diphenylphosphino)ferrocene
Et = ethyl
EtOAc = Ethyl acetate
FA = Formic acid
Fe = iron powder reduced
HATU = 2-(7-azaenzotriazol-1-yl)-N,N,N′,N′-
tetramethyluronium hexafluorophosphate
HPLC = high performance liquid chromatography
HPLC = High-performance liquid chromatography
LCMS = Liquid chromatography - mass spectrometry
LC-MS = liquid chromatography - mass spectrometry
LHS = left hand side
mCPBA = m-Chloroperoxybenzoic acid
Me = methyl
MeOH = Methanol
Ms = methanesulfonyl
Na₂CO₃ = Sodium carbonate anhydrous
Na₂SO₃ = Sodium sulfite anhydrous
Na₂SO₄ = Sodium sulfate anhydrous
NBS = N-bromosuccinimide
NMR = Nuclear magnetic resonance
PE = Petroleum ethergradient
PyBOP = benzotriazol-1-yl-oxytripyrrolidinophosphonium
hexafluorophosphate
RHS = right hand side
RT = retention time
SEM = 2-(trimethylsilyl) ethyl
Speedvac = Savant SC250EXP SpeedVac Concentrator
t-AmOH = 2-Methyl-2-butanol
TBAF = tetrabutylammonium fluoride
TBS = tertButylldimethylsilyl
TEA = triethylamine
TFA = Trifluoroacetic acid
THF = tetrahydrofuran

### Materials and Methods

The progress of reactions was often monitored by TLC or LC-MS. The identity of the products was often confirmed by LC-MS. The LC-MS was recorded using one of the following methods.

### LCMS Method AA:

EVO C18, 50 *3.0 mm, 0.1 μL injection, 1.2 mL/min flowrate, 90-900 amu scan range, 254 nm UV detection. Mobile Phase A (MPA): Water/5 mM NH4HCO3 and Mobile Phase B (MPB): Acetonitrile. Elution 20% MPB to 70% in 1.99 min, 70% MPB to 95% in 0.30 min, hold at 95% MPB for 0.4 min, 95% MPB to 10% in 0.3 min, then equilibration to 10% MPB for 0.2 min.

### LCMS Method AB:

Luna Omega, 50 *3 mm, 3.0 μL injection, 1.5 mL/min flowrate, 90-900 amu scan range, 254 nm UV detection. Mobile Phase A (MPA): Water/0.09% FA and Mobile Phase B (MPB): Acetonitrile/0.1% FA. Elution 5% MPB to 100% in 1.29 min, hold at 100% MPB for 0.9 min, 100% MPB to 5% in 0.05 min, then equilibration to 5% MPB for 0.25 min.

### LCMS Method AC:

Shim-pack XR-ODS, 50 *3 mm, 2.2 μL injection, 1.2 mL/min flowrate, 100-900 amu scan range, 254 nm UV detection. Mobile Phase A (MPA): Water/0.05% TFA and Mobile Phase B (MPB): Acetonitrile/0.05% TFA. Elution 50% MPB to 95% in 2.99 min, hold at 95% MPB for 0.6 min, 95% MPB to 5% in 0.1 min, then equilibration to 5% MPB for 0.25 min.

### LCMS Method BA

Instrument: Agilent LCMS system equiv. uipped with DAD and ELSD detector
Ion mode: Positive
Column: Waters X-Bridge C18, 50*2.1 mm*5 μm or equivalent
Mobile Phase: A: H₂O (0.04% TFA); B: CH₃CN (0.02% TFA)
Gradient: 4.5 min gradient method, actual method would depend on c log P of compound.
Flow Rate: 0.6 mL/min or 0.8 mL/min

### Column Temp: 40° C. or 50° C.

### UV: 220 nm

### LCMS Method BB

Instrument: Agilent LCMS system equipped with DAD and ELSD detector
Ion mode: Positive
Column: Waters X-Bridge ShieldRP18, 50*2.1 mm*5 μm or equivalent
Mobile Phase: A: H₂O (0.05% NH₃.H₂O) or 10 mM ammonia bicarbonate; B: CH₃CN
Gradient: 4.5 min gradient method; actual method would depend on the c log P of the compound.
Flow Rate: 0.6 mL/min or 0.8 mL/min

### Column Temp: 40° C.

### UV: 220 nm

LCMS Method CA: Kinetex EVO C18 100A, 30 *3 mm, 0.5 μL injection, 1.2 mL/min flowrate, 90-900 amu scan range, 254 nm UV detection. Mobile Phase A (MPA): Water/5 mM NH₄HCO₃ and Mobile Phase B (MPB): Acetonitrile. Elution 10% MPB to 95% in 2.0 min, hold at 95% MPB for 0.3 min, 95% MPB to 10% in 0.1 min.
LCMS Method CB: Xselect CSH C18, 50 *3 mm, 1.0 μL injection, 1.2 mL/min flowrate, 90-900 amu scan range, 254 nm UV detection. Mobile Phase A (MPA): Water/0.1% FA and Mobile Phase B (MPB): Acetonitrile/0.1% FA. Elution 5% MPB to 100% in 2.00 min, hold at 100% MPB for 0.7 min, 100% MPB to 5% in 0.05 min, then equilibration to 5% MPB for 0.15 min.
LCMS Method CC: XBridge Shield RP18, 50 *4.6 mm, 0.5 μL injection, 1.2 mL/min flowrate, 90-900 amu scan range, 254 nm UV detection. Mobile Phase A (MPA): Water/0.04% NH3.H2O and Mobile Phase B (MPB): Acetonitrile. Elution 10% MPB to 95% in 2.00 min, hold at 95% MPB for 0.79 min, 95% MPB to 10% in 0.06 min, then equilibration to 10% MPB for 0.15 min.
LCMS Method CD: HALO C18, 30 *3 mm, 0.8 μL injection, 1.2 mL/min flowrate, 90-900 amu scan range, 254 nm UV detection. Mobile Phase A (MPA): Water/0.1% FA and Mobile Phase B (MPB): Acetonitrile/0.05% FA. Elution 10% MPB to 100% in 1.30 min, hold at 100% MPB for 0.50 min, 100% MPB to 10% in 0.03 min, then equilibration to 10% MPB for 0.17 min.
LCMS Method CE: Shim-pack XR-ODS, 50 *3 mm, 0.3 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 100% in 1.10 min, hold at 100% MPB for 0.60 min, 100% MPB to 5% in 0.05 min, then equilibration to 5% MPB for 0.25 min.
LCMS Method CF: YMC-Triart C18, 30 *2 mm, 1.0 μL injection, 1.2 mL/min flowrate, 30-2000 amu scan range, 254 nm UV detection. Mobile Phase A (MPA): Water/0.05 TFA and Mobile Phase B (MPB): Acetonitrile/0.05% TFA. Elution 5% MPB to 95% in 1.00 min, hold at 95% MPB for 0.70 min, 95% MPB to 5% in 0.05 min, then equilibration to 5% MPB for 0.45 min.
LCMS Method CG: Kinetex 2.6 um EVO C18 100A, 50 *3 mm, 0.6 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.20 min, hold at 95% MPB for 0.50 min, 95% MPB to 10% in 0.05 min, then equilibration to 10% MPB for 0.10 min.
LCMS Method CH: Kinetex 2.6 um EVO C18 100A, 50 *3 mm, 0.6 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.20 min, hold at 95% MPB for 0.50 min, 95% MPB to 10% in 0.05 min, then equilibration to 10% MPB for 0.10 min.
LCMS Method CI: EVO C18, 50 *3 mm, 0.1 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 2.00 min, hold at 95% MPB for 0.60 min, 95% MPB to 10% in 015 min, then equilibration to 10% MPB for 0.25 min.
LCMS Method CJ: Shim-pack Scepter C18, 50 *3 mm, 0.8 μL injection, 1.5 mL/min flowrate, 30-2000 amu scan range, 254 nm UV detection. Mobile Phase A (MPA): Water/0.04% NH₃.H₂O and Mobile Phase B (MPB): Acetonitrile. Elution 10% MPB to 95% in 1.00 min, hold at 95% MPB for 0.60 min, 95% MPB to 10% in 0.03 min, then equilibration to 10% MPB for 0.17 min.
LCMS Method CK: Titank C18, 50 *3 mm, 0.5 μL injection, 1.5 mL/min flowrate, 30-2000 amu scan range, 254 nm UV detection. Mobile Phase A (MPA): Water/5 mM NH₄HCO₃ and Mobile Phase B (MPB): Acetonitrile. Elution 10% MPB to 95% in 1.80 min, hold at 95% MPB for 0.80 min, 95% MPB to 10% in 0.15 min, then equilibration to 10% MPB for 0.25 min.
LCMS Method CL: XBridge BEH C18, 50 *3 mm, 4.0 μL injection, 1.2 mL/min flowrate, 30-2000 amu scan range, 254 nm UV detection. Mobile Phase A (MPA): Water/5 mM NH₄HCO₃ and Mobile Phase B (MPB): Acetonitrile. Elution 5% MPB to 95% in 2.00 min, hold at 95% MPB for 0.70 min, 95% MPB to 5% in 0.05 min, then equilibration to 5% MPB for 0.25 min.
NMR: NMR was recorded on BRUKER NMR 300.03 Mz, DUL-C-H, ULTRASHIELD™ 300, AVANCE II 300 B-ACS™ 120 or BRUKER NMR 400.13 Mz, BBFO, ULTRASHIELD™ 400, AVANCE 111400, B-ACS™ 120.
Prep. HPLC condition

### Instrument:

### 1. GILSON 281 and Shimadzu LCMS 2010A

### 2. GILSON 215 and Shimadzu LC-20AP

### 3. GILSON 215

Mobile phase:
A: NH₄OH/H₂O=0.05% v/v; B: ACN
A: FA/H2=0.225% v/v; B: ACN

### Column

Xtimate C18 150*25 mm*5 μm
Flow rate: 25 mL/min or 30 mL/min
Monitor wavelength: 220&254 nm
Gradient: actual method would depend on clog P of compound

### Detector: MS Trigger or UV

### Preparation of Synthetic Intermediates

### Synthesis of Intermediate A1 (1-(5-amino-3-methylpyridin-2-yl)azetidin-3-ol)

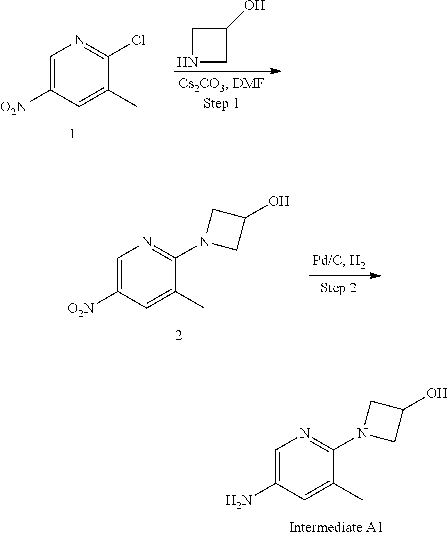

### Step 1: 1-(3-methyl-5-nitropyridin-2-yl)azetidin-3-ol

2-chloro-3-methyl-5-nitropyridine (600.0 mg, 3.5 mmol, 1.0 equiv.) was dissolved in DMF (30.0 mL), Cs₂CO₃ (4531.3 mg, 13.9 mmol, 4.0 equiv.) and azetidin-3-ol hydrochloride (380.9 mg, 3.5 mmol, 1.0 equiv.) were added. The reaction mixture was stirred for 6 hours at 60° C. and then quenched by the addition of water. The resulting solution was extracted with ethyl acetate, washed with brine, dried over anhydrous sodium sulfate and concentrated under vacuum to give 1-(3-methyl-5-nitropyridin-2-yl)azetidin-3-ol (500 mg, 68.7%) as a yellow solid. LCMS Method CC: [M−H]⁻=208.

### Step 2: 1-(5-amino-3-methylpyridin-2-yl)azetidin-3-ol

1-(3-methyl-5-nitropyridin-2-yl)azetidin-3-ol (450.0 mg, 2.2 mmol, 1.0 equiv.) was dissolved in MeOH (30.0 mL), Pd/C (90.0 mg, 10% wt., 0.1 mmol, 0.05 equiv.) was added. The resulting mixture was degassed and back filled with hydrogen for three times, then stirred for 3 hours at room temperature under atmosphere of hydrogen. The solids were filtered out and the resulting mixture was concentrated under vacuum to give 1-(5-amino-3-methylpyridin-2-yl)azetidin-3-ol (243.7 mg, 63.1%) as a brown solid. LCMS Method CD: [M+H]⁺=180.

The intermediates in the following table were prepared using the same method described for Intermediate A1.

| Inter- | Starting | Starting | | |
|---|---|---|---|---|
| mediate | material A | material B | Structure | LCMS data |
| Inter- mediate A2 | 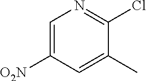 | 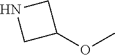 | 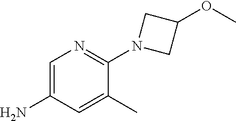 | Method CH: MS- ESI: 194 [M + H]⁺ |
| Inter- mediate A3 | 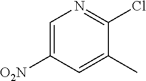 | 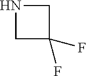 | 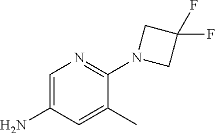 | Method CC: MS- ESI: 200 [M + H]⁺ |
| Inter- mediate A4 | 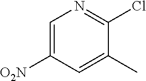 | 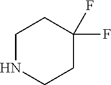 | 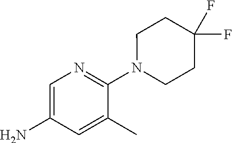 [SMILES: Cc1cc(N)cnc1N1CCC(F)(F)CC1] | Method CC: MS- ESI: 228 [M + H]⁺ |
| Inter- mediate A5 | 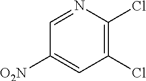 [SMILES: O=[N+]([O-])c1cnc(Cl)c(Cl)c1] | 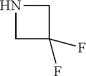 | 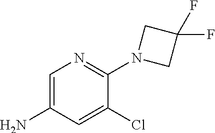 [SMILES: Nc1cnc(N2CC(F)(F)C2)c(Cl)c1] | Method CJ: MS- ESI: 220 [M + H]⁺ |
| Inter- mediate A6 | 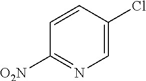 | 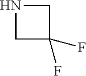 | 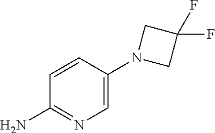 [SMILES: Nc1ccc(N2CC(F)(F)C2)cn1] | Method CC: MS- ESI: 186 [M + H]⁺ |
| Inter- mediate A7 | 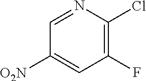 [SMILES: O=[N+]([O-])c1cnc(Cl)c(F)c1] | 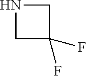 | 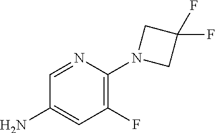 [SMILES: Nc1cnc(N2CC(F)(F)C2)c(F)c1] | Method CC: MS- ESI: 204 [M + H]⁺ |
| Inter- mediate A8 | 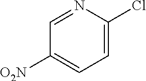 | 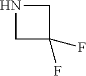 | 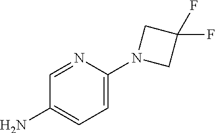 [SMILES: Nc1ccc(N2CC(F)(F)C2)nc1] | Method CJ: MS- ESI: 186 [M + H]⁺ |

### Synthesis of Intermediate A9 (6-(3,3-difluorocyclobutyl)-5-fluoropyridin-3-amine)

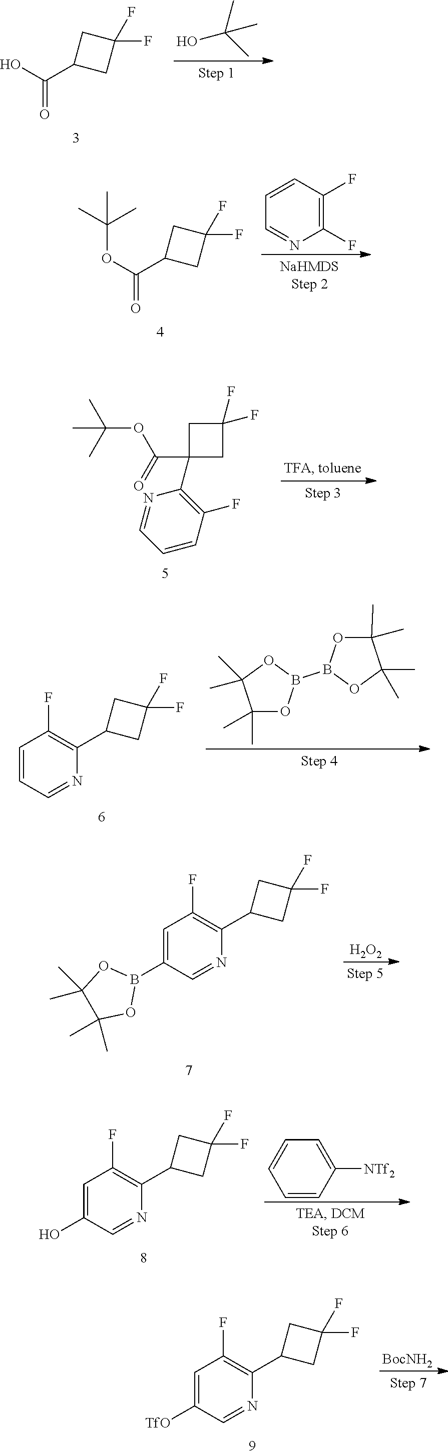

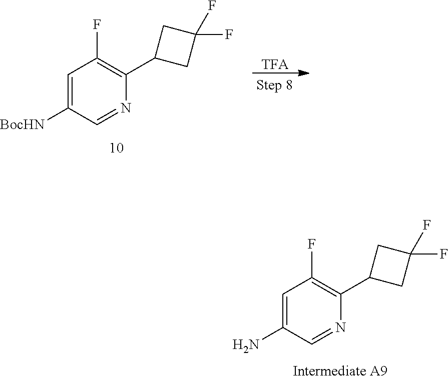 [SMILES: CC(C)(C)OC(=O)Nc1cnc(C2CC(F)(F)C2)c(F)c1 ; Nc1cnc(C2CC(F)(F)C2)c(F)c1 ; "TFA"]

### Step 1: tert-butyl 3,3-difluorocyclobutane-1-carboxylate

3,3-difluorocyclobutanecarboxylic acid (1.0 g, 7.3 mmol, 1.0 equiv.) was dissolved in DCM (10.0 mL), N,N-dimethylpyridin-4-amine (92.0 mg, 0.7 mmol, 0.1 equiv.), 2-methylpropan-2-ol (1.1 g, 14.7 mmol, 2.0 equiv.) and N,N′-dicyclohexylcarbodiimide (1.7 g, 8.1 mmol, 1.1 equiv.) were added at 10° C. The reaction mixture was warmed up to room temperature and stirred for 18 hours. The solid was removed by filtration and the filtrate was washed with 2N aqueous HCl solution, saturated aqueous NaHCO₃, brine, dried over anhydrous Na₂SO₄, and concentrated in vacuo to give crude tert-butyl 3,3-difluorocyclobutane-1-carboxylate (896.1 mg, 63.1%) as colorless oil. ¹H NMR (400 MHz, CDCl₃) δ 2.83-2.78 (m, 5H), 1.47 (s, 9H).

### Step 2: tert-butyl 3,3-difluoro-1-(3-fluoropyridin-2-yl)cyclobutane-1-carboxylate

2,3-difluoropyridine (1.2 g, 10.4 mmol, 1.0 equiv.) and tert-butyl 3,3-difluorocyclobutane-1-carboxylate (2.0 g, 10.4 mmol, 1.0 equiv.) were dissolved in toluene (60.0 mL). This was followed by the addition of NaHMDS (2 M in THF, 6.2 ml, 12.4 mmol, 1.2 equiv.) dropwise with stirring at 0° C. in 10 min. The resulting solution was stirred for 2 hours at 0° C. and then quenched by the addition of saturated aqueous NH₄C1. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give tert-butyl 3,3-difluoro-1-(3-fluoropyridin-2-yl)cyclobutane-1-carboxylate (1.6 g, 53.4%) as colorless oil. LCMS Method CD: [M+H]⁺=288.

### Step 3: 2-(3,3-difluorocyclobutyl)-3-fluoropyridine

tert-butyl 3,3-difluoro-1-(3-fluoropyridin-2-yl)cyclobutane-1-carboxylate (1.5 g, 5.2 mmol, 1.0 equiv.) was dissolved in DCM (30.0 mL), TFA (3.1 ml, 41.6 mmol, 8.0 equiv.) was added. The resulting solution was stirred for 10 hours at ambient temperature and then concentrated in vacuo. The residue was dissolved in toluene (30.0 mL) and stirred for 18 hours at 90° C. After cooling down to ambient temperature and quenching by addition of water, the pH value of the solution was adjusted to 7.5 with saturated aqueous Na₂CO₃. The solution was extracted with ethyl acetate, dried over anhydrous sodium sulfate and concentrated under vacuum. The residue was purified by flash column chromatography on silica gel, eluting with ethyl acetate/petroleum ether (1:7) to give 2-(3,3-difluorocyclobutyl)-3-fluoropyridine (700 mg, 71.6%) as colorless oil. LCMS Method CD: [M+H]⁺=188. ¹H NMR (400 MHz, DMSO-d₆): δ 8.45-8.43 (m, 1H), 7.69-7.67 (m, 1H), 7.40-7.38 (m, 1H), 3.72-3.70 (m, 1H), 3.02-2.85 (m, 4H).

### Step 4: 2-(3,3-difluorocyclobutyl)-3-fluoro-5-(4,4,5,5-tetramethyl-1,3,2-dioxaborolan-2-yl)pyridine

2-(3,3-difluorocyclobutyl)-3-fluoropyridine (700.0 mg, 3.7 mmol, 1.0 equiv.) was dissolved in heptane (30.0 mL), bis(pinacolato)diboron (1.1 g, 4.4 mmol, 1.2 equiv.), 4,4-di-tert-butyl-2,2-dipyridyl (1.0 g, 3.7 mmol, 1.0 equiv.) and di-methanolatodiiridium(Ir-Ir)-cycloocta-1,5-diene (1:2) (495.8 mg, 0.7 mmol, 0.2 equiv.) were added under atmosphere of nitrogen. The resulting solution was stirred for 18 hours at ambient temperature and then quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:5) to give 2-(3,3-difluorocyclobutyl)-3-fluoro-5-(4,4,5,5-tetramethyl-1,3,2-dioxaborolan-2-yl)pyridine (300 mg, 25.6%) as a white solid. LCMS Method CD: [M+H]⁺=314.

### Step 5: 6-(3,3-difluorocyclobutyl)-5-fluoropyridin-3-ol

2-(3,3-difluorocyclobutyl)-3-fluoro-5-(4,4,5,5-tetramethyl-1,3,2-dioxaborolan-2-yl)pyridine (300.0 mg, 0.9 mmol, 1.0 equiv.) was dissolved in MeOH (10.0 mL) and H₂O (3.0 mL). Then H₂O₂(30%, 0.14 ml, 1.4 mmol, 1.5 equiv.) was added. The resulting solution was stirred for 30 min at ambient temperature and then quenched by the addition of saturated aqueous Na₂S₂O₃. The resulting solution was extracted with ethyl acetate, dried over anhydrous sodium sulfate and concentrated under vacuum. The residue was purified by flash column chromatography on silica gel, eluting with ethyl acetate/petroleum ether (1:2) to give 6-(3,3-difluorocyclobutyl)-5-fluoropyridin-3-ol (160 mg, 82.2%) as a white solid. LCMS Method CD: [M+H]⁺=204. ¹H NMR (400 MHz, CD₃OD-d₄): δ 8.0 (s, 1H), 6.97-6.93 (m, 1H), 3.69-3.58 (m, 1H), 3.01-2.78 (m, 4H).

### Step 6: 6-(3,3-difluorocyclobutyl)-5-fluoropyridin-3-yl trifluoromethanesulfonate

6-(3,3-difluorocyclobutyl)-5-fluoropyridin-3-ol (160.0 mg, 0.7 mmol, 1.0 equiv.), was dissolved in DCM (20.0 mL), TEA (0.1 ml, 0.9 mmol, 1.2 equiv.) and 1,1,1-trifluoro-N-phenyl-N-trifluoromethanesulfonylmethanesulfonamide (309.4 mg, 0.8 mmol, 1.1 equiv.) were added. The resulting solution was stirred for 30 min at ambient temperature and then quenched by the addition of water. The solution was extracted with ethyl acetate, dried over anhydrous sodium sulfate and concentrated under vacuum. The residue was purified by flash column chromatography on silica gel, eluting with ethyl acetate/petroleum ether (1:8) to give 6-(3,3-difluorocyclobutyl)-5-fluoropyridin-3-yltrifluoromethanesulfonate (220 mg, 83.3%) as a white solid. LCMS Method CD: [M+H]⁺=336.

### Step 7: tert-butyl (6-(3,3-difluorocyclobutyl)-5-fluoropyridin-3-yl)carbamate

6-(3,3-difluorocyclobutyl)-5-fluoropyridin-3-yltrifluoromethanesulfonate (220.0 mg, 0.6 mmol, 1.0 equiv.) was dissolved in 1,4-dioxane (30.0 mL). Then NH₂Boc (230.3 mg, 1.9 mmol, 3.0 equiv.), 4,5-Bis(diphenylphosphino)-9,9-dimethylxanthene (75.8 mg, 0.1 mmol, 0.2 equiv.) and Pd₂(dba)₃ (120.1 mg, 0.1 mmol, 0.2 equiv.) were added under nitrogen. The resulting solution was stirred for 3 hours at 90° C. under atmosphere of nitrogen and then concentrated under vacuum. The residue was purified by flash column chromatography on silica gel, eluting with ethyl acetate/petroleum ether (1:9) to give tert-butyl (6-(3,3-difluorocyclobutyl)-5-fluoropyridin-3-yl)carbamate (120 mg, 60.4%) as a white solid. LCMS Method CD: [M+H]⁺=303.

### Step 8: 6-(3,3-difluorocyclobutyl)-5-fluoropyridin-3-amine

tert-Butyl N-[6-(3,3-difluorocyclobutyl)-5-fluoropyridin-3-yl]carbamate (120.0 mg, 0.3 mmol, 1.0 equiv.) was dissolved in DCM (10.0 mL), TFA (2.0 ml) was added. The resulting solution was stirred for 30 min at ambient temperature and then diluted with water. The pH value of the solution was adjusted to 7.5 with saturated aqueous Na₂CO₃ and extracted with ethyl acetate. The organic layer was dried over anhydrous sodium sulfate and concentrated under vacuum. The residue was purified by flash column chromatography on silica gel, eluting with ethyl acetate/petroleum ether (1:3) to give 6-(3,3-difluorocyclobutyl)-5-fluoropyridin-3-amine (60 mg, 74.7%) as a white solid. LCMS Method CD: [M+H]⁺=203.

### Synthesis of Intermediate A10 (2-(2-(trifluoromethyl)phenoxy)ethan-1-amine)

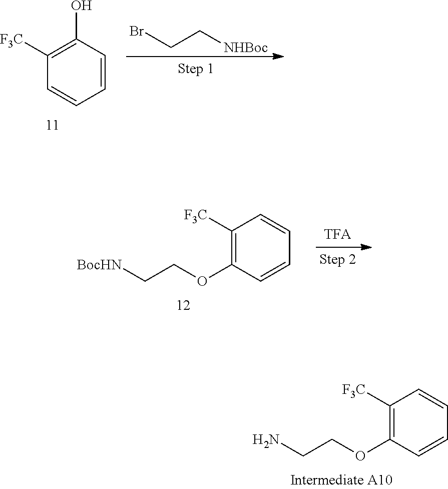

### Step 1: tert-butyl (2-(2-(trifluoromethyl)phenoxy)ethyl)carbamate

2-(trifluoromethyl)phenol (2.0 g, 12.3 mmol, 1.0 equiv.) was dissolved DMF (20.0 mL), Cs₂CO₃ (8.0 g, 24.7 mmol, 2.0 equiv.), NaI (5.6 g, 0.1 mmol, 3 equiv.) and tert-butyl N-(2-bromoethyl)carbamate (11.1 g, 49.3 mmol, 4.0 equiv.) was added. The resulting solution was stirred for 10 hours at 80° C. and then concentrated under vacuum. The residue was purified by flash column chromatography on silica gel, eluting with ethyl acetate/petroleum ether (1:1) to give tert-butyl (2-(2-(trifluoromethyl)phenoxy)ethyl)carbamate (1.5 g, 39.8%) as a yellow solid. LCMS Method CI: [M+H]⁺=306.

### Step 2: 2-(2-(trifluoromethyl)phenoxy)ethan-1-amine

tert-butyl [2-[2-(trifluoromethyl)phenoxy]ethyl]carbamate (1.0 g, 3.3 mol, 1.0 equiv.) was dissolved in DCM (10.0 mL), then TFA (2.0 ml) was added. The resulting solution was stirred for 30 min at ambient temperature and then diluted with water. The pH value of the solution was adjusted to 7.5 with saturated aqueous Na₂CO₃ and extracted with ethyl acetate. The organic layer was dried over anhydrous sodium sulfate and concentrated under vacuum to give 2-[2-(trifluoromethyl)phenoxy]ethanamine (500 mg, 74.4%) as a yellow solid. LCMS Method CD: [M+H]⁺=206.

The intermediates in the following table were prepared using the same method described for Intermediate A10.

| | Starting material | | |
|---|---|---|---|
| Intermediate | Used | Structure | LCMS data |
| Intermediate A11 | 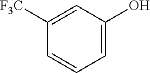 [SMILES: Oc1cccc(C(F)(F)F)c1] | 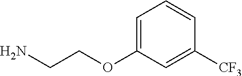 [SMILES: NCCOc1cccc(C(F)(F)F)c1] | Method CD: MS- ESI: 206 [M + H]⁺ |

### Synthesis of Intermediate A12 (spiro[5.5]undecan-3-amine)

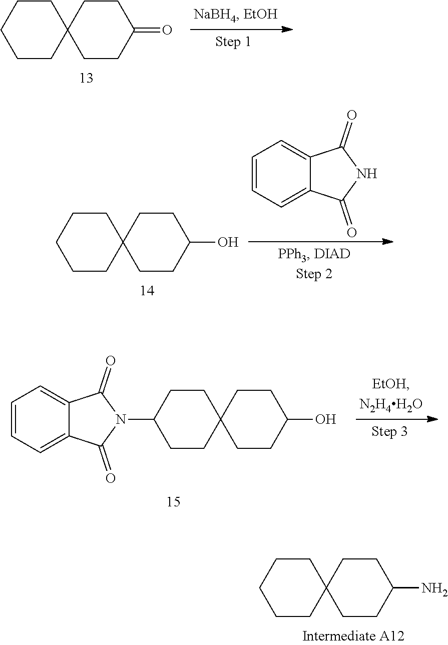 [SMILES: O=C1CCC2(CCCCC2)CC1 ; OC1CCC2(CCCCC2)CC1 ; O=C1NC(=O)c2ccccc21 ; O=C1c2ccccc2C(=O)N1C1CCC2(CCC(O)CC2)CC1 ; NC1CCC2(CCCCC2)CC1]

### Step 1: spiro[5.5]undecan-3-ol

Spiro[5.5]undecan-3-one (1.0 g, 6.0 mmol, 1.0 equiv.) was dissolved in EtOH (20.0 mL), NaBH₄ (682.6 mg, 18.0 mmol, 3.0 equiv.) was added in portions. The resulting mixture was stirred for 2 hours at room temperature under atmosphere of nitrogen and then quenched by the addition of water. The aqueous layer was extracted with ethyl acetate, dried over anhydrous Na₂SO₄ and concentrated under vacuum. The residue was purified by flash column chromatography on silica gel, eluting with ethyl acetate/petroleum ether (1:1) to give spiro[5.5]undecan-3-ol (1.1 g, 82.6%) as a yellow oil. LCMS Method CD: [M+H]⁺=169.

### Step 2: 2-(spiro[5.5]undecan-3-yl)isoindoline-1,3-dione

Spiro[5.5]undecan-3-ol (1.0 g, 5.9 mmol, 1.0 equiv.) and phthalimide (1.3 g, 8.9 mmol, 1.5 equiv.) were dissolved in THF (20.0 mL), PPh₃ (3.1 g, 11.9 mmol, 2.0 equiv.) was added. This was followed by the addition of DIAD (1.2 g 5.9 mmol, 1.0 equiv.) in portions at room temperature. The resulting mixture was stirred for 8 hours at room temperature under nitrogen atmosphere and then quenched by the addition of ice-water. The resulting solution was extracted with ethyl acetate and concentrated under vacuum. The residue was purified by flash column chromatography on silica gel, eluting with ethyl acetate/petroleum ether (1:6) to give 2-[spiro[5.5]undecan-3-yl]isoindole-1,3-dione (998 mg, 45.7%) as a off-white solid. LCMS Method CF: [M+H]⁺=298.

### Step 3: spiro[5.5]undecan-3-amine

2-[spiro[5.5]undecan-3-yl]isoindole-1,3-dione (748.0 mg, 2.5 mmol, 1.0 equiv.) was dissolved in EtOH (25.0 mL), N2H₄.H₂O (251.8 mg, 5.0 mmol, 2.0 equiv.) was added at room temperature. The resulting mixture was stirred for 5 hours at 80° C. under nitrogen atmosphere. The resulting mixture was filtered, and the filtrate was concentrated under vacuum to give crude spiro[5.5]undecan-3-amine (797 mg) as a yellow solid. LCMS Method CF: [M+H]⁺=168.

### Synthesis of Intermediate A13 (4-(3,3-difluorocyclobutyl)-3-fluoroaniline)

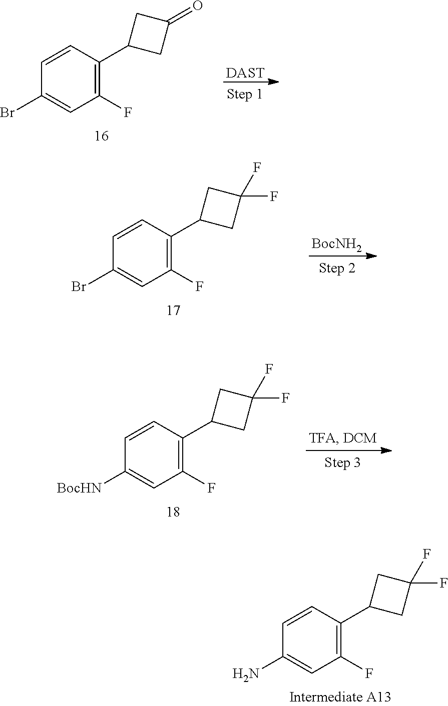

### Step 1: 4-bromo-1-(3,3-difluorocyclobutyl)-2-fluorobenzene

3-(4-Bromo-2-fluorophenyl)cyclobutan-1-one (1.3 g, 5.3 mmol, 1.0 equiv.) was dissolved in DAST (30.0 mL) at 0° C. under atmosphere of nitrogen. The resulting mixture was stirred for overnight at room temperature and then quenched by the addition of aqueous NaHCO₃ at 0° C. The resulting mixture was extracted with DCM, dried over anhydrous Na₂SO₄ and concentrated under vacuum. The residue was purified by flash column chromatography on silica gel, eluting with ethyl acetate/petroleum ether (1:2) to give 4-bromo-1-(3,3-difluorocyclobutyl)-2-fluorobenzene (1.1 g) as a yellow oil. ¹H NMR (300 MHz, DMSO-d₄): δ 7.53-7.49 (m, 1H), 7.43-7.34 (m, 2H), 3.52-3.46 (m, 1H), 3.07-2.94 (m, 2H), 2.84-2.66 (m, 2H).

### Step 2: tert-butyl (4-(3,3-difluorocyclobutyl)-3-fluorophenyl)carbamate

4-Bromo-1-(3,3-difluorocyclobutyl)-2-fluorobenzene (1.1 g, 4.2 mmol, 1.0 equiv.) and BocNH₂ (2.4 g, 20.7 mmol, 5.0 equiv.) were dissolved in toluene (11.0 mL). Pd₂(dba)₃ (0.4 g, 0.4 mmol, 0.1 equiv.), XPhos (0.4 g, 0.8 mmol, 0.2 equiv.) and t-BuOK (2.3 g, 20.7 mmol, 5.0 equiv.) were added at room temperature under atmosphere of nitrogen. The resulting mixture was stirred for overnight at 100° C. and then quenched by the addition of water. The resulting solution was extracted with ethyl acetate, dried over anhydrous sodium sulfate and concentrated under vacuum. The residue was purified by flash column chromatography on silica gel, eluting with ethyl acetate/petroleum ether (1:8) to give tert-butyl [4-(3,3-difluorocyclobutyl)-3-fluorophenyl]carbamate (1.0 g, 80.0%) as a white solid. LCMS Method CA: [M+H]⁺=302.

### Step 3: 4-(3,3-difluorocyclobutyl)-3-fluoroaniline

tert-Butyl [4-(3,3-difluorocyclobutyl)-3-fluorophenyl]carbamate (1.2 g, 4.0 mmol, 1.0 equiv.) was dissolved in DCM (12.0 mL), TFA (3.0 mL) was added dropwise at 0° C. The resulting mixture was stirred for 2 hours at room temperature and then concentrated under vacuum. The residue was dissolved in DCM, and the solution was washed with sat. NaHCO₃ aqueous and brine, dried over anhydrous sodium sulfate and concentrated under vacuum to give crude 4-(3,3-difluorocyclobutyl)-3-fluoroaniline (800 mg) as a red oil. LCMS Method CA: [M+H]⁺=202.

### Synthesis of Intermediate A14 (cyclopentyl 6-amino-1H-indole-4-carboxylate)

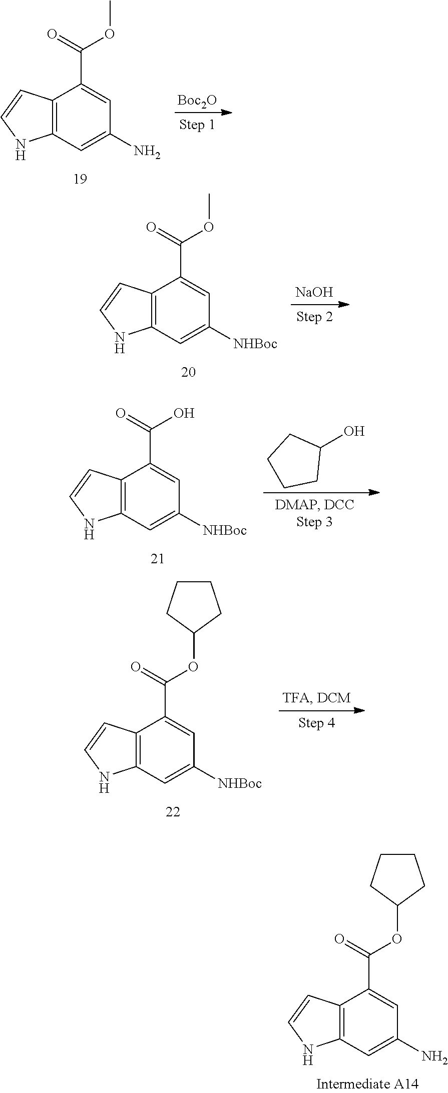

### Step 1: methyl 6-((tert-butoxycarbonyl)amino)-1H-indole-4-carboxylate

Methyl 6-amino-1H-indole-4-carboxylate (5.0 g, 26.3 mmol, 1.0 equiv.) was dissolved in THF (100.0 mL)/aqueous NaOH (4M, 25.0 mL), Boc₂O (8.6 g, 39.4 mmol, 1.5 equiv.) was added. The resulting solution was stirred for 3 hours at room temperature. The mixture was extracted with ethyl acetate, washed with brine and concentrated under vacuum. The residue was purified by flash column chromatography on silica gel, eluting with acetate/petroleum ether (1:4) to give methyl 6-((tert-butoxycarbonyl)amino)-1H-indole-4-carboxylate (4.1 g, 53.9%) as a white solid. LCMS Method CA: [M+H]⁺=291.

### Step 2: 6-((tert-butoxycarbonyl)amino)-1H-indole-4-carboxylic acid

Methyl 6-((tert-butoxycarbonyl)amino)-1H-indole-4-carboxylate (1.0 g, 3.4 mmol, 1.0 equiv.) was dissolved in MeOH/water (10.0 mL/10.0 mL), NaOH (0.7 g, 17.2 mmol, 5.0 equiv.) was added. The reaction mixture was stirred for 4 hours at room temperature. The pH value of the solution was adjusted to 6 with aqueous HCl solution (2M), then extracted with ethyl acetate, washed with brine, dried over anhydrous Na₂SO₄ and concentrated under vacuum to give 6-((tert-butoxycarbonyl)amino)-1H-indole-4-carboxylic acid (520 mg, 54.6%) as a white solid. LCMS Method CC: [M−H]⁻=275.

### Step 3: cyclopentyl 6-((tert-butoxycarbonyl)amino)-1H-indole-4-carboxylate

6-[(tert-butoxycarbonyl)amino]-1H-indole-4-carboxylic acid (480.0 mg, 1.7 mmol, 1.0 equiv.) and cyclopentanol (299.2 mg, 3.5 mmol, 2.0 equiv.) were dissolved in DCM (8.0 mL), DMAP (106.1 mg, 0.9 mmol, 0.5 equiv.) and DCC (716.9 mg, 3.5 mmol, 2.0 equiv.) were added. The solution was stirred for 3 hours at room temperature and then concentrated under vacuum. The residue was purified by flash column chromatography on silica gel, eluting with ethyl acetate/petroleum ether (1:1) to give cyclopentyl 6-[(tert-butoxycarbonyl)amino]-1H-indole-4-carboxylate (240 mg, 40.1%) as a off-white solid. LCMS Method CJ: [M+H]⁺=345.

### Step 4: cyclopentyl 6-amino-1H-indole-4-carboxylate

Cyclopentyl 6-[(tert-butoxycarbonyl)amino]-1H-indole-4-carboxylate (240.0 mg, 0.7 mmol, 1.0 equiv.) was dissolved in DCM/TFA (3 mL/1 mL). The resulting mixture was stirred for 4 hours at room temperature and then concentrated under vacuum. The residue was purified by flash column chromatography on silica gel, eluting with DCM/MeOH (10:1) to give cyclopentyl 6-amino-1H-indole-4-carboxylate (200 mg) as a off-white solid. LCMS Method CJ: [M+H]⁺=245.

### Synthesis of Intermediate A15 (4-(2-((tert-butyldimethylsilyl)oxy)ethyl)-1H-indol-6-amine)

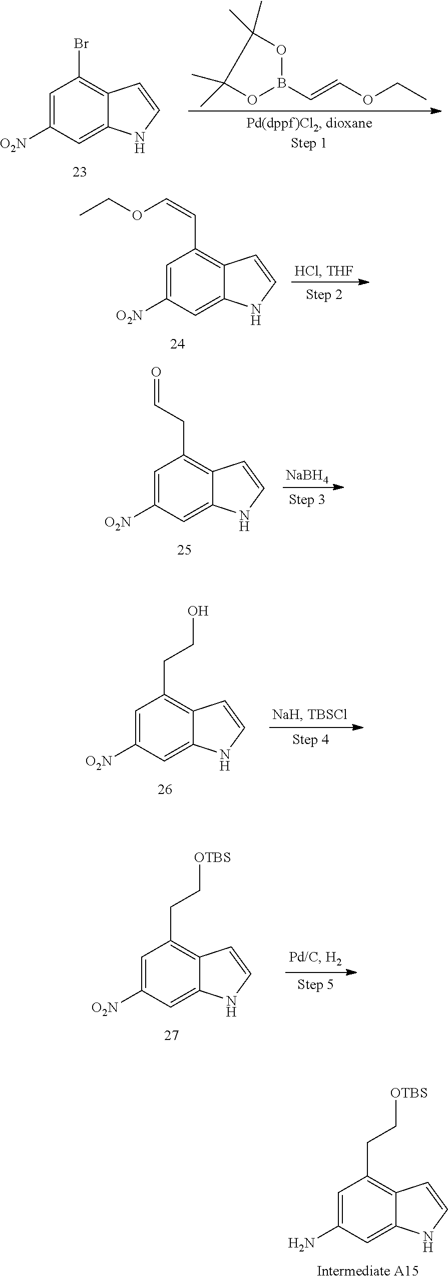

### Step 1: (Z)-4-(2-ethoxyvinyl)-6-nitro-1H-indole

4-Bromo-6-nitro-1H-indole (3.0 g, 12.4 mmol, 1.0 equiv.) was dissolved in dioxane/water (150.0 mL/30.0 mL), (E)-2-(2-ethoxyvinyl)-4,4,5,5-tetramethyl-1,3,2-dioxaborolane (3.7 g, 18.7 mmol, 1.5 equiv.), K₂CO₃ (3440.2 mg, 24.9 mmol, 2.0 equiv.) and Pd(dppf)Cl₂ (910.7 mg, 1.2 mmol, 0.1 equiv.) were added under nitrogen. The reaction mixture was stirred for 6 hours at 90° C. under atmosphere of nitrogen and then concentrated under vacuum. The residue was purified by flash column chromatography on silica gel, eluting with ethyl acetate/petroleum ether (1:3) to give (Z)-4-(2-ethoxyvinyl)-6-nitro-1H-indole (3.0 g) as a orange solid. LCMS Method CD: [M+H]⁺=233.

### Step 2/3: 2-(6-nitro-1H-indol-4-yl)ethan-1-ol

4-[(Z)-2-ethoxyethenyl]-6-nitro-1H-indole (2.0 g, 8.6 mmol, 1.0 equiv.) was dissolved in DCM/TFA (200.0 mL/20 mL). The reaction mixture was stirred for 2 hours at room temperature and concentrated under vacuum to give crude 2-(6-nitro-1H-indol-4-yl)acetaldehyde. The residue was dissolved in MeOH (10.0 mL), and NaBH₄ (741.2 mg, 19.6 mmol, 2.0 equiv.) was added in portions. The reaction mixture was stirred for additional 2 hours at room temperature and then quenched by the addition of water. The resulting mixture was extracted with ethyl acetate, dried over anhydrous Na₂SO₄ and concentrated under vacuum. The residue was purified by flash column chromatography on silica gel, eluting with DCM/MeOH (10:1) to give 2-(6-nitro-1H-indol-4-yl)ethan-1-ol (600 mg, 29.7%) as brown oil. LCMS Method CD: [M+H]⁺=207.

### Step 4: 4-(2-((tert-butyldimethylsilyl)oxy)ethyl)-6-nitro-1H-indole

2-(6-nitro-1H-indol-4-yl)ethan-1-ol (600.0 mg, 2.9 mmol, 1.0 equiv.) was dissolved in THF (30.0 mL), NaH (60% wt in mineral oil, 232.8 mg, 5.8 mmol, 2.0 equiv.) was added under atmosphere of nitrogen. After stirred for 30 min, TBSCl (657.9 mg, 4.4 mmol, 1.5 equiv.) was added. The reaction mixture was stirred for 2 hours and then quenched by the addition of 10 mL of MeOH. The resulting mixture was concentrated under vacuum and the residue was purified by flash column chromatography on silica gel, eluting with DCM/MeOH (10:1) to give 4-(2-((tert-butyldimethylsilyl)oxy)ethyl)-6-nitro-1H-indole (560 mg, 60.1%) as yellow oil. LCMS Method CC: [M+H]⁺=321.

### Step 5: 4-(2-((tert-butyldimethylsilyl)oxy)ethyl)-1H-indol-6-amine

4-(2-((tert-butyldimethylsilyl)oxy)ethyl)-6-nitro-1H-indole (500.0 mg, 1.6 mmol, 1.0 equiv.) was dissolved in MeOH (30.0 mL), Pd/C (50.0 mg, 0.5 mmol, 0.3 equiv.) was added. The resulting mixture was degassed and back filled with hydrogen for three times, and then stirred for 2 hours under atmosphere of hydrogen. The mixture was filtered through Celite and the filtrate was concentrated under vacuum to give crude 4-(2-((tert-butyldimethylsilyl)oxy)ethyl)-1H-indol-6-amine (450 mg) as a off-white solid. LCMS Method CD: [M+H]⁺=291.

### Synthesis of Intermediate A16 (4-(2-methoxyethyl)-1H-indol-6-amine)

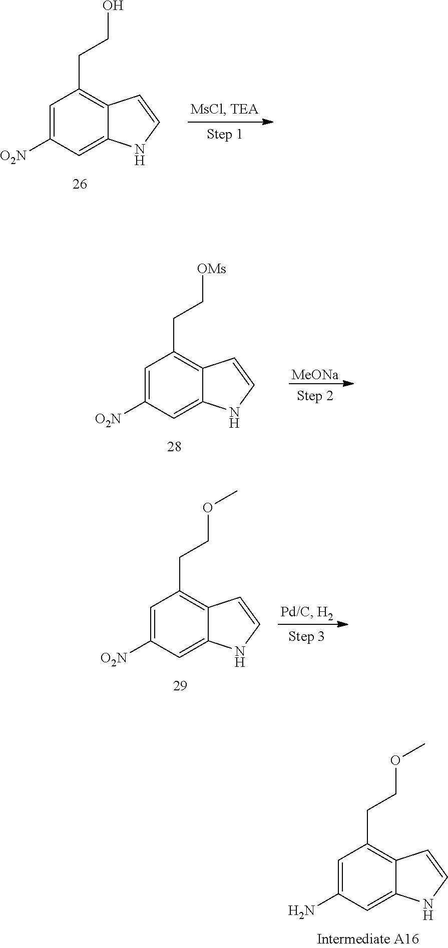 [SMILES: "MeONa" ; O=[N+]([O-])c1cc(CCO)c2cc[nH]c2c1 ; CS(=O)(=O)OCCc1cc([N+](=O)[O-])cc2[nH]ccc12 ; COCCc1cc([N+](=O)[O-])cc2[nH]ccc12 ; "Pd/C, H2" ; COCCc1cc(N)cc2[nH]ccc12]

### Step 1/2: 4-(2-methoxyethyl)-6-nitro-1H-indole

2-(6-Nitro-1H-indol-4-yl)ethanol (300.0 mg, 1.5 mmol, 1.0 equiv.) and TEA (441.6 mg, 4.4 mmol, 3.0 equiv.) were dissolved in THF (10.0 mL), MsCl (249.9 mg, 2.2 mmol, 1.5 equiv.) was added dropwise at 0° C. under atmosphere of nitrogen. The resulting mixture was stirred for 5 min at room temperature, then to the above mixture was added MeONa/MeOH (30% wt, 10.0 mL) dropwise at 0° C. The resulting mixture was stirred for additional overnight at room temperature and concentrated under vacuum. The resulting mixture was concentrated under vacuum and the residue was purified by flash column chromatography on silica gel, eluting with ethyl acetate/petroleum ether (1:2) to give 4-(2-methoxyethyl)-6-nitro-1H-indole (101 mg, 31.5%) as a brown solid. LCMS Method CC: [M+H]⁺=221.

### Step 3: 4-(2-methoxyethyl)-1H-indol-6-amine

4-(2-Methoxyethyl)-6-nitro-1H-indole (100.0 mg, 0.5 mmol, 1.0 equiv.) was dissolved in THF (4.0 mL), Pd/C (48.3 mg, 0.5 mmol, 1.0 equiv.) was added. The resulting mixture was degassed and back filled with hydrogen for three times, then stirred for 3 hours at room temperature under atmosphere of hydrogen. The resulting mixture was filtered through Celite and the filtrate was concentrated under vacuum to give 4-(2-methoxyethyl)-1H-indol-6-amine (67 mg, 72.2%) as a orange crude solid. LCMS Method CI: [M+H]⁺=191.

### Synthesis of Intermediate A17 (2-(6-amino-1H-indol-4-yl)ethyl methylcarbamate)

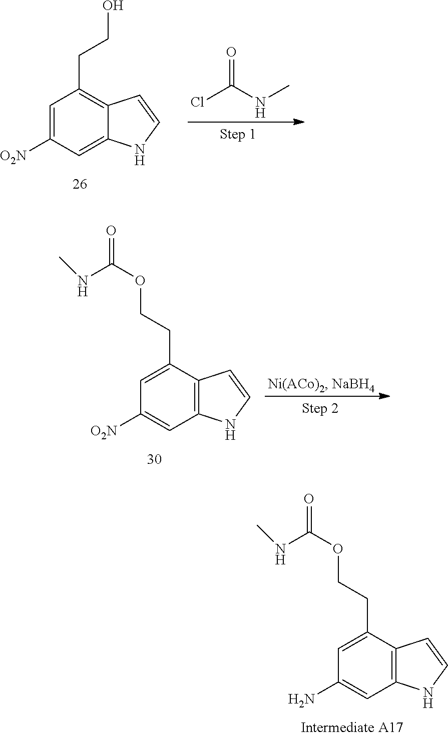

### Step 1: 2-(6-nitro-1H-indol-4-yl)ethyl methylcarbamate

2-(6-Nitro-1H-indol-4-yl)ethanol (190.0 mg, 0.9 mmol, 1.0 equiv.) was dissolved THF (10.0 mL), TEA (0.3 mL, 1.8 mmol, 2.0 equiv.), DMAP (225.1 mg, 1.8 mmol, 2.0 equiv.) and N-methylcarbamoyl chloride (103.4 mg, 1.1 mmol, 1.2 equiv.) were added. The resulting solution was stirred for 12 hours at room temperature and then concentrated under vacuum. The resulting mixture was concentrated under vacuum and the residue was purified by flash column chromatography on silica gel, eluting with ethyl acetate/petroleum ether (1:10) to give 2-(6-nitro-1H-indol-4-yl)ethyl methylcarbamate (130 mg, 53.6%) as yellow oil. LCMS Method CD: [M+H]⁺=264.

### Step 2: 2-(6-amino-1H-indol-4-yl)ethyl methylcarbamate

2-(6-nitro-1H-indol-4-yl)ethyl methylcarbamate (120.0 mg, 0.5 mmol, 1.0 equiv.) and Ni(AcO)₂ (156.8 mg, 0.9 mmol, 2.0 equiv.) were dissolved in MeOH (10.0 mL), NaBH₄ (69.0 mg, 1.8 mmol, 4.0 equiv.) was added under 0° C. The resulting solution was stirred for 1 hour at 0° C. and then quenched by the addition of water. The resulting solution was extracted with ethyl acetate, dried over anhydrous sodium sulfate and concentrated under vacuum. The resulting mixture was concentrated under vacuum and the residue was purified by flash column chromatography on silica gel, eluting with ethyl acetate/petroleum ether (1:8) to give 2-(6-amino-1H-indol-4-yl)ethyl N-methylcarbamate (40 mg, 37.6%) as yellow oil. LCMS Method CD: [M+H]⁺=234. ¹H NMR (400 MHz, DMSO-d₆): δ 9.04 (s, 1H), 8.45-8.42 (m, 2H), 8.18 (s, 1H), 7.96 (s, 1H), 6.98 (brs, 2H), 4.27 (t, 2H), 3.24 (t, 2H), 2.88 (s, 3H).

### Synthesis of Intermediate A18 (4-(2-(dimethylamino)ethyl)-1H-indol-6-amine)

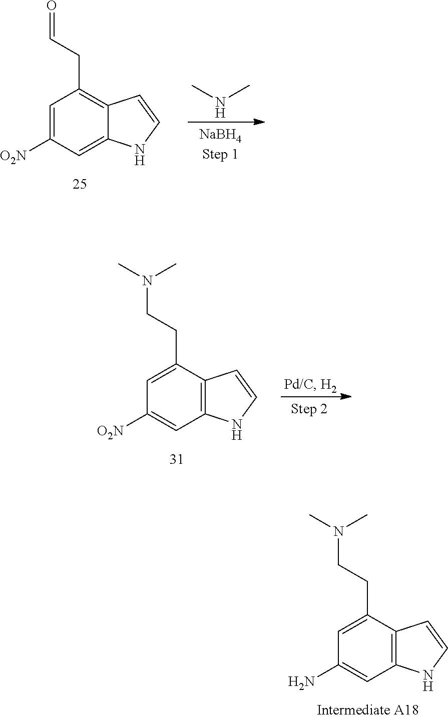

### Step 1: N,N-dimethyl-2-(6-nitro-1H-indol-4-yl)ethan-1-amine

2-(6-Nitro-1H-indol-4-yl)acetaldehyde (340.0 mg, 1.7 mmol, 1.0 equiv.) was dissolved in MeOH (5.0 mL), dimethylamine (2M in THF, 1.0 mL, 2.0 mmol, 1.2 equiv.) was added. This was followed by the addition of NaBH₄ (111.2 mg, 3.0 mmol, 2.0 equiv.) in portions. The reaction mixture was stirred for 2 hours at room temperature and then quenched by the addition of water. After concentration, the residue was purified by flash column chromatography on silica gel, eluting with ethyl acetate/petroleum ether (1:1) to give N,N-dimethyl-2-(6-nitro-1H-indol-4-yl)ethan-1-amine (220 mg, 64.2%) as a brown oil. LCMS Method CC: [M+H]⁺=234.

### Step 2: 4-(2-(dimethylamino)ethyl)-1H-indol-6-amine

N,N-dimethyl-2-(6-nitro-1H-indol-4-yl)ethan-1-amine (200.0 mg, 0.9 mmol, 1.0 equiv.) was dissolved in MeOH (40.0 mL), then Pd/C (10.0 mg, 0.1 mmol, 0.1 equiv.) was added. The reaction mixture was degassed and back filled with hydrogen for three times, then stirred for 2 hours under atmosphere of hydrogen. The reaction mixture was filtered through Celite and the filtrate was concentrated under vacuum to give 4-(2-(dimethylamino)ethyl)-1H-indol-6-amine (150 mg, 86.1%) as colorless oil. LCMS Method CC: [M+H]⁺=204.

### Synthesis of Intermediate A19 (6-amino-N-methyl-1H-indole-3-sulfonamide)

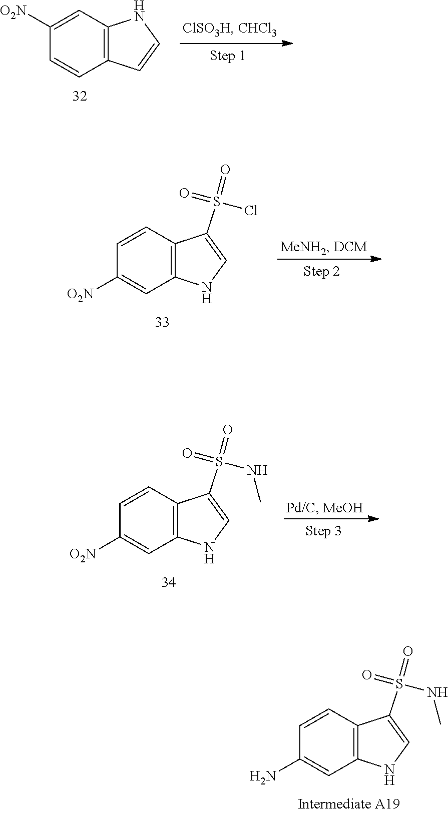

### Step 1: 6-nitro-1H-indole-3-sulfonyl chloride

To a mixture of HSO₃Cl (9 mL) and DCM (90 mL), was added Na₂SO₄ (2.1 g, 14.8 mmol, 1.0 equiv.). This was followed by the addition of a solution of 6-nitro-1H-indole (2.4 g, 14.8 mmol, 1.0 equiv.) in DCM (40.0 mL) dropwise at room temperature. The resulting solution was stirred for 30 min at room temperature and decanted provide a thick brown oil. The color oil was slowly treated with water/ice, the solids were collected by filtration and dried to give 6-nitro-1H-indole-3-sulfonyl chloride (1.7 g, crude) as a brown solid.

### Step 2: N-methyl-6-nitro-1H-indole-3-sulfonamide

6-Nitro-1H-indole-3-sulfonyl chloride (1.7 g, 6.5 mmol, 1.0 equiv.) was dissolved in THF (10.0 mL), methylamine in THF (2M, 16 mL, 8.0 mmol, 1.2 equiv.) was added and the resulting solution was stirred for 14 hours at room temperature. After concentration under vacuum, the residue was purified by flash column chromatography on silica gel, eluting with ethyl acetate/petroleum ether (1:2) to give N-methyl-6-nitro-1H-indole-3-sulfonamide (500 mg) as a yellow solid. LCMS Method CA: [M−H]⁻=254.

### Step 3: 6-amino-N-methyl-1H-indole-3-sulfonamide

N-methyl-6-nitro-1H-indole-3-sulfonamide (500.0 mg, 1.9 mmol, 1.0 equiv.) was dissolved in MeOH/THF (10/5 mL), Pd/C (10% wt, 176.0 mg, 0.2 mmol, 0.1 equiv.) was added. The reaction mixture was degassed and back filled with hydrogen for three times, then stirred for 10 hours under atmosphere of hydrogen. The reaction mixture was filtered through Celite and the filtrate was concentrated under vacuum. The residue was purified by flash column chromatography on silica gel, eluting with dichloromethane/methanol (10:1) to give 6-amino-N-methyl-1H-indole-3-sulfonamide (450 mg) as a yellow solid. LCMS Method CA: [M−H]⁻=224.

### Synthesis of Intermediate 20 (6-amino-N-methyl-1H-indole-3-sulfonamide)

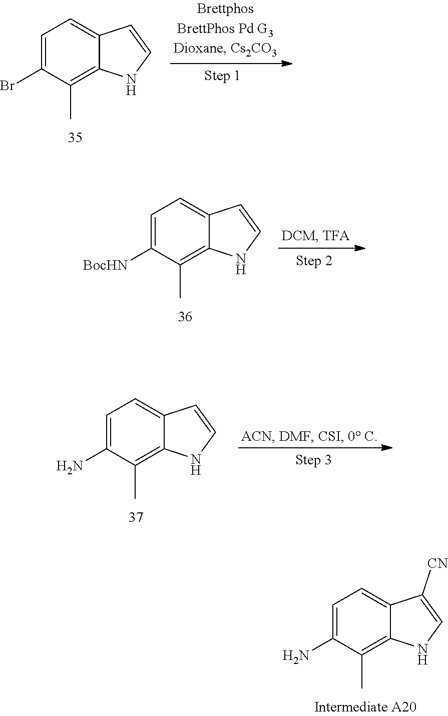

### Step 1: tert-butyl (7-methyl-1H-indol-6-yl)carbamate

6-Bromo-7-methyl-1H-indole (200.0 mg, 1.0 mmol, 1.0 equiv.) was dissolved in dioxane (20.0 mL), BrettPhos Pd G3 (86.3 mg, 0.1 mmol, 0.1 equiv.), Brettphos (51.1 mg, 0.1 mmol, 0.1 equiv.) and Cs₂CO₃ (620.4 mg, 1.9 mmol, 2.0 equiv.) were added at room temperature under nitrogen. The resulting mixture was stirred for 8 hours at 105° C. under atmosphere of nitrogen and concentrated under vacuum. The residue was purified by flash column chromatography on silica gel, eluting with dichloromethane/methanol (10:1) to give tert-butyl (7-methyl-1H-indol-6-yl)carbamate (200 mg, 85.0%) as a off-white solid. LCMS Method CI: [M+H]⁺=247. ¹H NMR (300 MHz, DMSO-d₆): δ 10.96 (s, 1H), 8.45 (brs, 1H), 7.29-7.25 (m, 2H), 6.86-6.82 (m, 1H), 6.38-6.36 (m, 1H), 2.30 (s, 3H), 1.45 (s, 9H).

### Step 2: 7-methyl-1H-indol-6-amine

tert-Butyl (7-methyl-1H-indol-6-yl)carbamate (200.0 mg, 0.8 mmol, 1.0 equiv.) was dissolved in DCM/TFA (30 mL/5 mL) at room temperature. The resulting mixture was stirred for 5 min at room temperature and quenched by the addition of water. The pH value of the solution was adjusted to 8 with saturated NaHCO₃ aqueous. The resulting solution was extracted with ethyl acetate and concentrated under vacuum to give crude 7-methyl-1H-indol-6-amine (110 mg) as yellow oil. LCMS Method CC: [M+H]⁺=147.

### Step 3: 6-amino-7-methyl-1H-indole-3-carbonitrile

7-methyl-1H-indol-6-amine (100.0 mg, 0.7 mmol, 1.0 equiv.) and CSI (116.6 mg, 0.8 mmol, 1.2 equiv.) were dissolved in ACN (15.0 mL), DMF (0.4 mL mg, 4.8 mmol, 7.0 equiv.) was added dropwise 0° C. The resulting mixture was stirred for 2.5 hours at 0° C. under atmosphere of nitrogen and then quenched by the addition of water. The resulting mixture was extracted with ethyl acetate, washed with brine, dried over anhydrous Na₂SO₄ and concentrated under vacuum to give 6-amino-7-methyl-1H-indole-3-carbonitrile (90.0 mg, 76.8%) as a brown solid. LCMS Method CC: [M+H]⁺=172.

The intermediates in the following table were prepared using the same method described for Intermediate A20.

| | Starting material | | |
|---|---|---|---|
| Intermediate | Used | Structure | LCMS data |
| Intermediate A21 | 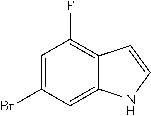 [SMILES: Fc1cc(Br)cc2[nH]ccc12] | 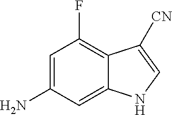 [SMILES: N#Cc1c[nH]c2cc(N)cc(F)c12] | Method CI: MS- ESI: 176 [M + H]⁺ |

### Synthesis of Intermediate A22 (6-amino-1-(2-methylpyridin-3-yl)-1H-indole-3-carbonitrile)

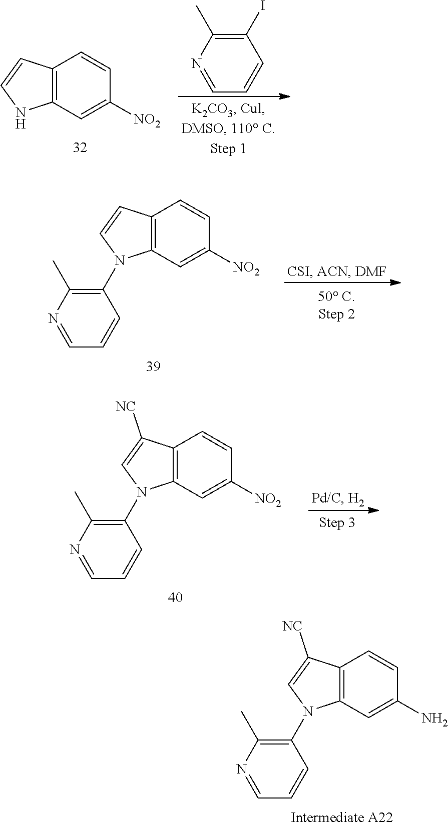

### Step 1: 1-(2-methylpyridin-3-yl)-6-nitroindole

6-nitro-1H-indole (1.5 g, 9.3 mmol, 1.0 equiv.) and 3-iodo-2-methylpyridine (4.1 g, 18.5 mmol, 2.0 equiv.) were dissolved in DMSO (36.0 mL), K₂CO₃ (2.6 g, 18.5 mmol, 2.0 equiv.), 8-hydroxyquinoline (268.6 mg, 1.9 mmol, 0.2 equiv.) and CuI (352.4 mg, 1.9 mmol, 0.2 equiv.) were added under nitrogen. The resulting mixture was stirred for overnight at 110° C. and then quenched by the addition of water. The solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:4) to give 1-(2-methylpyridin-3-yl)-6-nitroindole (990 mg, 42.3%) as a yellow solid. LCMS Method CD: [M+H]⁺=254.

### Step 2: 1-(2-methylpyridin-3-yl)-6-nitroindole-3-carbonitrile

1-(2-Methylpyridin-3-yl)-6-nitroindole (330.0 mg, 1.3 mmol, 1.0 equiv.) was dissolved in ACN (6.0 mL), CSI (184.4 mg, 1.3 mmol, 1.0 equiv.) was added at 0° C. under atmosphere of nitrogen. The resulting mixture was stirred for 2 hours at 50° C., then to the above mixture was added DMF (0.7 mL, 9.4 mmol, 7.2 equiv.) at 0° C. The resulting mixture was stirred for additional 1.5 hours at room temperature and concentrated under vacuum. The residue was purified by reverse flash chromatography with the following conditions: column, C18 silica gel; mobile phase, NH₄HCO₃ in water, 30% to 80% MeCN in 30 min; detector, UV 254 nm. This resulted in 1-(2-methylpyridin-3-yl)-6-nitroindole-3-carbonitrile (228 mg, 62.9%) as a yellow solid. LCMS Method CI: [M+H]⁺=279.

### Step 3: 6-amino-1-(2-methylpyridin-3-yl)indole-3-carbonitrile

1-(2-Methylpyridin-3-yl)-6-nitroindole-3-carbonitrile (220.0 mg, 0.8 mmol, 1.0 equiv.) was dissolved in MeOH (5.0 mL), Pd/C (16.8 mg, 0.2 mmol, 0.2 equiv.) was added. The resulting mixture was degassed and back filled with hydrogen for three times, then stirred for 1 hour at room temperature under atmosphere of hydrogen. The reaction mixture was filtered through Celite and the filtrate was concentrated under vacuum to give 6-amino-1-(2-methylpyridin-3-yl)indole-3-carbonitrile (190 mg, 96.8%) as a yellow solid. LCMS Method CJ: [M+H]⁺=279.

### Synthesis of Intermediate A23 (I-acetyl-6-amino-1H-indole-3-carbonitrile)

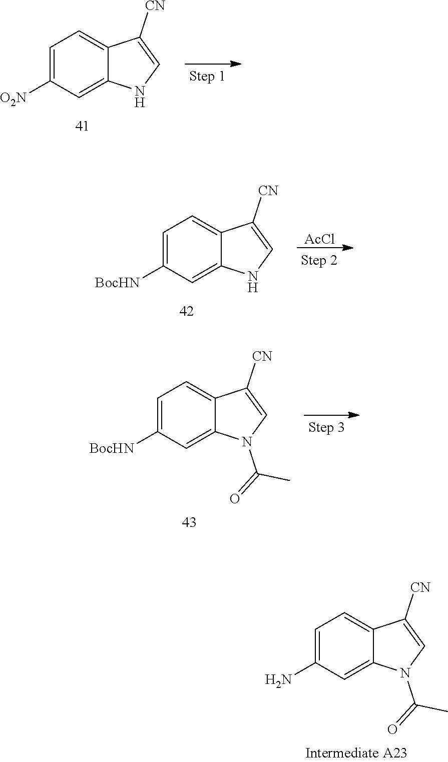 [SMILES: "AcCl" ; N#Cc1c[nH]c2cc([N+](=O)[O-])ccc12 ; CC(C)(C)OC(=O)Nc1ccc2c(C#N)c[nH]c2c1 ; CC(=O)n1cc(C#N)c2ccc(NC(=O)OC(C)(C)C)cc21 ; CC(=O)n1cc(C#N)c2ccc(N)cc21]

### Step 1: tert-butyl (3-cyano-1H-indol-6-yl)carbamate

6-Nitro-1H-indole-3-carbonitrile (500.0 mg, 2.7 mmol, 1.0 equiv.) was dissolved in THF (10.0 mL), NiCl₂.6H₂O (71.4 mg, 0.3 mmol, 0.1 equiv.) and Boc₂O (1.2 g 5.4 mmol, 2.0 equiv.) were added. This was followed by the addition of NaBH₄ (123.1 mg, 3.2 mmol, 1.2 equiv.) under 0° C. The resulting solution was stirred for 8 hours at room temperature and concentrated under vacuum. The residue was purified by flash column chromatography on silica gel, eluting with ethyl acetate/petroleum ether (1:4) to give tert-butyl (3-cyano-1H-indol-6-yl)carbamate (210 mg, 30.6%) as a off-white solid. LCMS Method CD: [M+H]⁺=258.

### Step 2: tert-butyl (I-acetyl-3-cyano-1H-indol-6-yl)carbamate

tert-Butyl (3-cyano-1H-indol-6-yl)carbamate (210 mg, 0.8 mmol, 1.0 equiv.) and TEA (0.2 mL, 1.6 mmol, 2.0 equiv.) were dissolved in THF (5.0 mL), AC₂O (326.4 mg, 3.2 mmol, 4.0 equiv.) was added at 0° C. The reaction mixture was stirred for 2 hours at room temperature and quenched by the addition of water. The resulting solution was extracted with ethyl acetate, washed with brine, dried over anhydrous Na₂SO₄ and concentrated under vacuum to give tert-butyl (I-acetyl-3-cyano-1H-indol-6-yl)carbamate (210 mg, 85.9%) as a yellow solid. LCMS Method CD: [M+H]⁺=300.

### Step 3: 1-acetyl-6-amino-1H-indole-3-carbonitrile

tert-Butyl (1-acetyl-3-cyano-1H-indol-6-yl)carbamate (200 mg, 0.7 mmol, 1.0 equiv.) was dissolved in HCl/dioxane (4M, 5.0 mL). The reaction solution was stirred for 2 hours at room temperature and quenched by the addition of water. The pH value of the solution was adjusted to 9 with saturated aqueous Na₂CO₃ solution and then extracted with ethyl acetate, dried over anhydrous Na₂SO₄ and concentrated under vacuum to give 1-acetyl-6-amino-1H-indole-3-carbonitrile (104 mg, 78.1%) as a brown solid. LCMS Method CD: [M+H]⁺=200.

### Synthesis of Intermediate A24 (6-amino-4-methyl-1H-indole-3-carbonitrile)

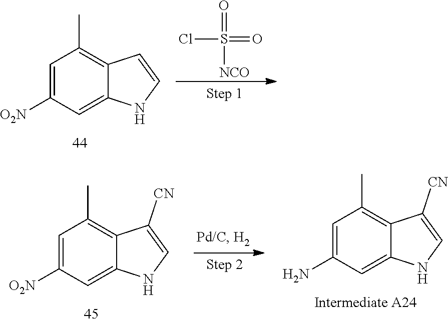

### Step 1: 4-methyl-6-nitro-1H-indole-3-carbonitrile

4-Methyl-6-nitro-1H-indole (500.0 mg, 2.8 mmol, 1.0 equiv.) was dissolved in ACN (20.0 mL), CSI (482.0 mg, 3.4 mmol, 1.2 equiv.) was added at 0° C. The resulting mixture was stirred for 2 hours at 0° C., then to the above mixture was added DMF (0.4 mL, 5.7 mmol, 2.0 equiv.) dropwise at 0° C. The resulting mixture was stirred for additional 2 hours at 0° C. and quenched by the addition of water. The resulting mixture was extracted with ethyl acetate, washed with brine, dried over anhydrous Na₂SO₄ and concentrated under vacuum to give 4-methyl-6-nitro-1H-indole-3-carbonitrile (420 mg, 73.56%) as a yellow solid. LCMS Method CI: [M+H]⁺=202.

### Step 2: 6-amino-4-methyl-1H-indole-3-carbonitrile

4-Methyl-6-nitro-1H-indole-3-carbonitrile (400.0 mg, 2.0 mmol, 1.0 equiv.) was dissolved in MeOH (10.0 mL), Pd/C (105.8 mg, 1.0 mmol, 0.5 equiv.) was added. The reaction mixture was degassed and back filled with hydrogen for three times, then stirred for 2 hours at room temperature under atmosphere of hydrogen. After filtration, the resulting mixture was concentrated under vacuum to give 6-amino-4-methyl-1H-indole-3-carbonitrile (280 mg, 82.3%) as a yellow solid. LCMS Method CI: [M+H]⁺=172.

### Synthesis of Intermediate A25 (6-amino-4-methyl-1H-indole-3-carbonitrile)

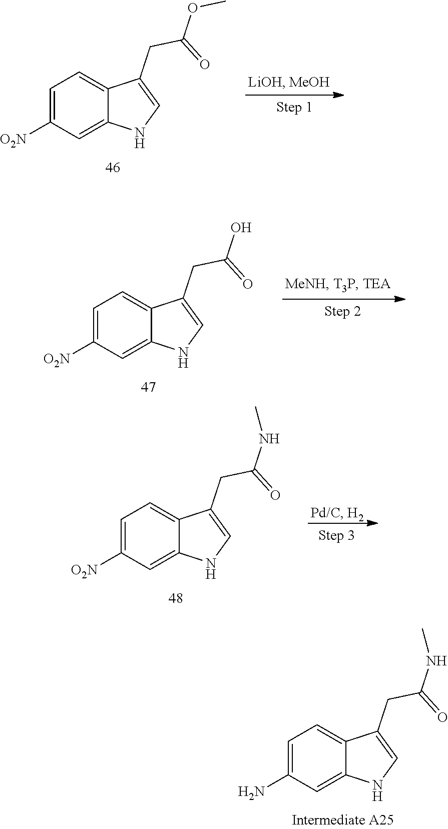 [SMILES: COC(=O)Cc1c[nH]c2cc([N+](=O)[O-])ccc12 ; O=C(O)Cc1c[nH]c2cc([N+](=O)[O-])ccc12 ; CNC(=O)Cc1c[nH]c2cc([N+](=O)[O-])ccc12 ; CNC(=O)Cc1c[nH]c2cc(N)ccc12]

### Step 1: 2-(6-Nitro-1H-indol-3-yl)acetic acid

2-(6-Nitro-1H-indol-3-yl)acetate (500.0 mg, 2.0 mmol, 1.0 equiv.) was dissolved in MeOH/water (5.0 mL/1.0 mL), LiOH.H₂O (422.6 mg, 10.1 mmol, 5.0 equiv.) was added. The resulting solution was stirred for 3 hours at room temperature. The pH vale of the solution was adjusted to 6 with aqueous HCl solution (4M). The resulting mixture was extracted with ethyl acetate, washed with brine, dried over anhydrous Na₂SO₄ and concentrated under vacuum to give (6-nitro-1H-indol-3-yl)acetic acid (300 mg, 67.6%) as a off-white solid. LCMS Method CJ: [M−H]⁻=219.

### Step 2: N-methyl-2-(6-nitro-1H-indol-3-yl)acetamide

(6-Nitro-1H-indol-3-yl)acetic acid (500.0 mg, 2.3 mmol, 1.0 equiv.) was dissolved THF (5.0 mL), CH₃NH₂.HCl (184.1 mg, 2.7 mmol, 1.2 equiv.), T₃P (2167.6 mg, 6.8 mmol, 3.0 equiv.) and TEA (0.6 mL, 4.5 mmol, 2.0 equiv.) were added. The resulting solution was stirred for 3 hours at room temperature and concentrated under vacuum. The residue was purified by flash column chromatography on silica gel, eluting with ethyl acetate/petroleum ether (1:1) to give N-methyl-2-(6-nitro-1H-indol-3-yl)acetamide (400 mg, 75.5%) as a off-white solid. LCMS Method CC: [M+H]⁺=234.

### Step 3: 2-(6-amino-1H-indol-3-yl)-N-methylacetamide

N-methyl-2-(6-nitro-1H-indol-3-yl)acetamide (500.0 mg, 2.1 mmol, 1.0 equiv.) was dissolved in MeOH (10.0 mL), Pd/C (222.8 mg, 2.1 mmol, 1.0 equiv.) was added. The reaction mixture was degassed and back filled with hydrogen and then stirred for 10 min at room temperature under atmosphere of hydrogen. After filtration and concentration, the residue was purified by flash column chromatography on silica gel, eluting with ethyl acetate/petroleum ether (1:1) to give 2-(6-amino-1H-indol-3-yl)-N-methylacetamide (350 mg, 90.0%) as a off-white solid. LCMS Method CC: [M+H]⁺=204.

### Synthesis of Intermediate A26 (6-amino-4-methyl-1H-indole-3-carbonitrile)

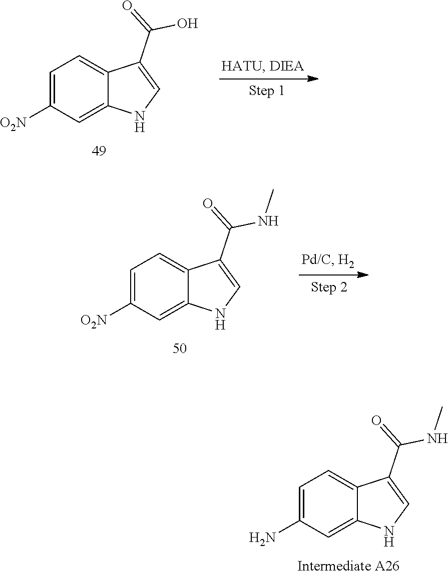 [SMILES: O=C(O)c1c[nH]c2cc([N+](=O)[O-])ccc12 ; CNC(=O)c1c[nH]c2cc([N+](=O)[O-])ccc12 ; CNC(=O)c1c[nH]c2cc(N)ccc12]

### Step 1: N-methyl-6-nitro-1H-indole-3-carboxamide

6-Nitro-1H-indole-3-carboxylic acid (300.0 mg, 1.5 mmol, 1.0 equiv.) and methylamine hydrogen chloride (110.0 mg, 1.6 mmol, 1.1 equiv.) were dissolved in THF (20.0 mL), HATU (553.3 mg, 1.5 mmol, 1.0 equiv.), DIEA (0.5 mL, 2.9 mmol, 2.0 equiv.) was added. The resulting mixture was stirred for 4 hours at room temperature and concentrated under vacuum. The residue was purified by flash column chromatography on silica gel, eluting with DCM/MeOH (10:1) to give N-methyl-6-nitro-1H-indole-3-carboxamide (240 mg, 75.2%) as a off-white solid. LCMS Method CC: [M+H]⁺=220.

### Step 2: 6-amino-N-methyl-1H-indole-3-carboxamide

N-methyl-6-nitro-1H-indole-3-carboxamide (200.0 mg, 0.9 mmol, 1.0 equiv.) was dissolved in MeOH (0 mL), Pd/C (wt 10%, 100 mg, 0.1 mmol, 0.1 equiv.) was added. The reaction mixture was degassed and back filled with hydrogen and then stirred for 2 hours at room temperature under atmosphere of hydrogen. After filtration, the filtrate was concentrated under vacuum to give crude 6-amino-N-methyl-1H-indole-3-carboxamide (187 mg) as a off white solid. LCMS Method CC: [M+H]⁺=190.

### Synthesis of Intermediate A27 (6-amino-N-methyl-1H-indole-4-carboxamide)

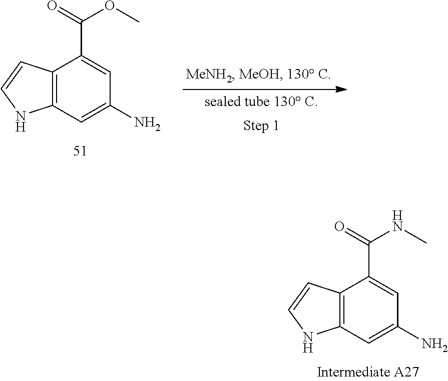

Methyl 6-amino-1H-indole-4-carboxylate (250.0 mg, 1.3 mmol, 1.0 equiv.) was dissolved in MeOH (5.0 mL), CH₃NH₂/THF solution (5.0 mL, 1M, 5.0 mmol, 4.0 equiv.) was added at room temperature. The resulting mixture was stirred for overnight at 130° C. and then concentrated under vacuum. The residue was purified by flash column chromatography on silica gel, eluting with DCM/MeOH (10:1) to give 6-amino-N-methyl-1H-indole-4-carboxamide (150 mg, 60.3%) as a orange solid. LCMS Method CF: [M+H]⁺=190.

### Synthesis of Intermediate A28 (6-amino-1-(1-methyl-1H-pyrazol-4-yl)-1H-indole-3-carbonitrile)

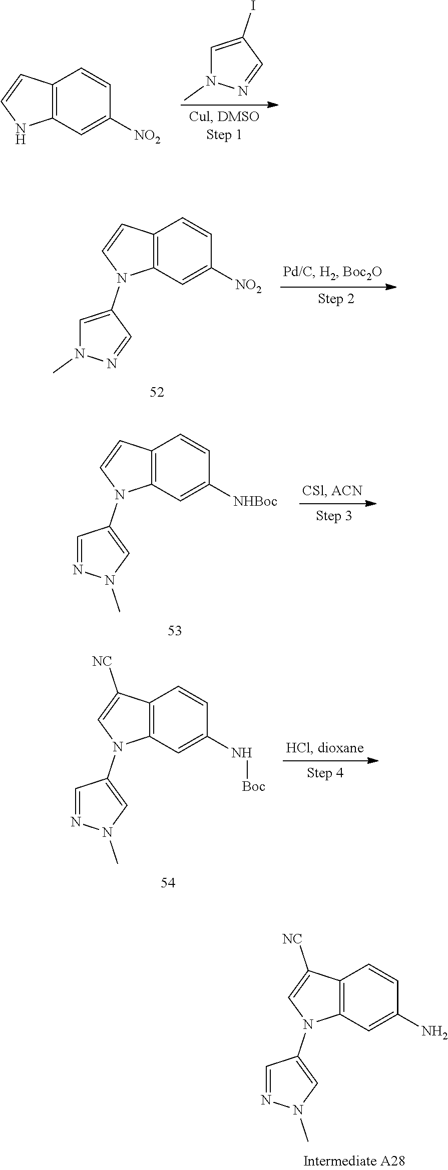

### Step 1: 1-(1-methyl-1H-pyrazol-4-yl)-6-nitro-1H-indole

6-Nitro-1H-indole (1.0 g, 6.2 mmol, 1.0 equiv.) and 4-iodo-1-methyl-1H-pyrazole (2.6 g, 12.5 mmol, 2.0 equiv.) were dissolved in DMSO (20.0 mL), K₂CO₃ (1.7 g, 12.3 mmol, 2.0 equiv.), CuI (233.0 mg, 1.2 mmol, 0.2 equiv.) and quinolin-8-ol (179.0 mg, 1.2 mmol, 0.2 equiv.) were added under air atmosphere. The reaction mixture was stirred for 2 hours at 110° C. and concentrated under vacuum. The residue was purified by flash column chromatography on silica gel, eluting with ethyl acetate/petroleum ether (1:1) to give 1-(1-methyl-1H-pyrazol-4-yl)-6-nitro-1H-indole (916.0 mg, 61.3%) as an off-white solid. LCMS Method CE: [M+H]⁺=243.

### Step 2: tert-butyl (1-(1-methyl-1H-pyrazol-4-yl)-1H-indol-6-yl) carbamate

1-(1-methyl-1H-pyrazol-4-yl)-6-nitro-1H-indole (700.0 mg, 2.9 mmol, 1.0 equiv.) and Boc₂O (812.0 mg, 3.7 mmol, 1.3 equiv.) was dissolved in THF (20.0 mL), Pd/C (10%, wt, 100.00 mg, 0.1 mmol, 0.03 equiv.) was added. The reaction mixture was degassed and back filled with hydrogen for three times and then stirred for 3 hours at room temperature under atmosphere of hydrogen. After filtration to remove the solid, the filtrate was concentrated under vacuum. The residue was purified by flash column chromatography on silica gel, eluting with ethyl acetate/petroleum ether (1:1) to give tert-butyl N-[1-(1-methylpyrazol-4-yl) indol-6-yl] carbamate (630.0 mg) as a brown solid. LCMS Method CJ: [M+H]⁺=313.

### Step 3: tert-butyl (3-cyano-1-(1-methyl-1H-pyrazol-4-yl)-1H-indol-6-yl) carbamate

tert-butyl N-[1-(1-methylpyrazol-4-yl)indol-6-yl] carbamate (600.0 mg, 1.9 mmol, 1.0 equiv.) and CSI (273.1 mg, 1.9 mmol, 1.0 equiv.) were dissolved in ACN (20.0 mL). After stirred for 2 hours at room temperature, to the above mixture was added DMF (1.0 mL, 13.5 mmol, 7.0 equiv.) dropwise at 0° C. The solution was stirred for additional 2 hours at room temperature, then the pH value of the solution was adjusted to 6 by dropwise adding aqueous NaOH (1 mol/L). The solution was extracted with ethyl acetate, washed with brine, dried over anhydrous Na₂SO₄ and concentrated under vacuum. The residue was purified by flash column chromatography on silica gel, eluting with ethyl acetate/petroleum ether (1:1) to give tert-butyl N-[3-cyano-1-(1-methylpyrazol-4-yl) indol-6-yl] carbamate (300.0 mg) as an off-white solid. LCMS Method CI: [M+H]⁺=338.

### Step 4: 6-amino-1-(1-methyl-1H-pyrazol-4-yl)-1H-indole-3-carbonitrile

tert-butyl N-[3-cyano-1-(1-methylpyrazol-4-yl) indol-6-yl] carbamate (300.0 mg, 0.9 mmol, 1.0 equiv.) was dissolved in HCl/1,4-dioxane (4M, 10.0 mL). The resulting solution was stirred for 2 hours, and the pH value was adjusted to 8 by dropwise adding aqueous NaOH (1 mol/L). The aqueous layer was extracted with ethyl acetate and concentrated under vacuum to give 6-amino-1-(1-methyl-1H-pyrazol-4-yl)-1H-indole-3-carbonitrile (200.0 mg) as an off-white solid. LCMS Method CI: [M+H]⁺=238.

### Synthesis of Intermediate A29 (3-(1-methyl-1H-pyrazol-4-yl)-1H-indol-6-amine)

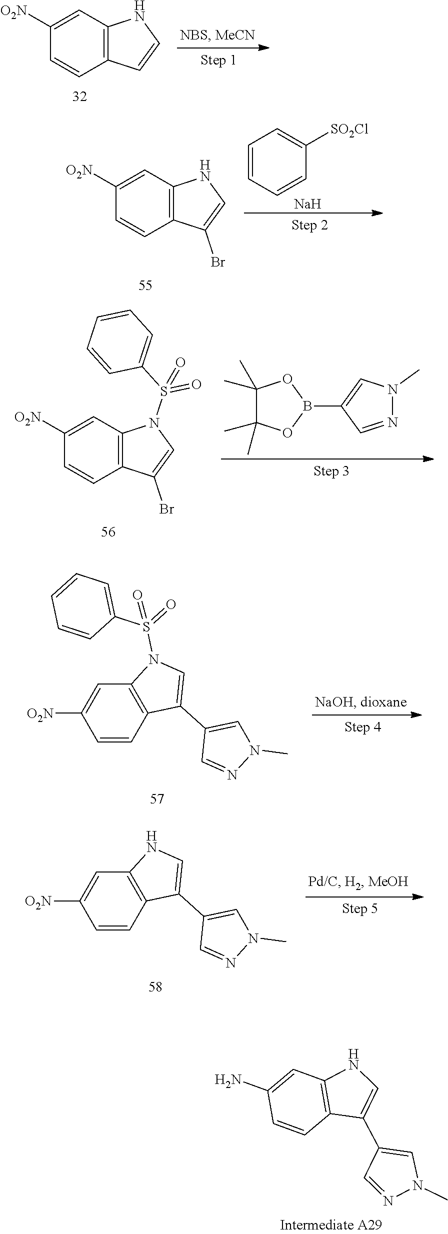

### Step 1: 3-bromo-6-nitro-1H-indole

6-Nitro-1H-indole (300.0 mg, 1.9 mmol, 1.0 equiv.) was dissolved in MeCN (5.0 mL), NBS (395.1 mg, 2.2 mmol, 1.2 equiv.) was added in portions at 0° C. The resulting mixture was stirred for overnight at room temperature under nitrogen atmosphere and then quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:3) to give 3-bromo-6-nitro-1H-indole (150 mg, 33.6%) as a red solid. LCMS Method CF: [M+H]⁺=241.

### Step 2: 3-bromo-6-nitro-1-(phenylsulfonyl)-1H-indole

3-Bromo-6-nitro-1H-indole (100.0 mg, 0.4 mmol, 1.0 equiv.) was dissolved in THF (2.0 mL), NaH (60% wt in mineral oil, 33.0 mg, 0.8 mmol, 2.0 equiv.) was added in portions at 0° C. under atmosphere of nitrogen. After stirred for 30 min, to above mixture was added benzenesulfonyl chloride (110.0 mg, 0.6 mmol, 1.5 equiv.) at 0° C. The resulting mixture was stirred for additional overnight at room temperature and then quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:5) to give 3-bromo-6-nitro-1-(phenylsulfonyl)-1H-indole (100 mg, 63.2%) as a white solid. LCMS Method CB: [M+H]⁺=381.

### Step 3: 3-(1-methyl-1H-pyrazol-4-yl)-6-nitro-1-(phenylsulfonyl) -1H-indole

3-Bromo-6-nitro-1-(phenylsulfonyl)-1H-indole (100.0 mg, 0.3 mmol, 1.0 equiv.) and 1-methyl-4-(4,4,5,5-tetramethyl-1,3,2-dioxaborolan-2-yl)pyrazole (273.0 mg, 1.3 mmol, 5.0 equiv.) were dissolved in dioxane/water (2.0 mL/0.2 mL), Pd(dppf)Cl₂ (19.0 mg, 0.03 mmol, 0.1 equiv.) and Cs₂CO₃ (256.1 mg, 0.8 mmol, 3.0 equiv.) were added under atmosphere of nitrogen. The resulting mixture was stirred for overnight at 90° C. under nitrogen atmosphere and then quenched by the addition of water. The resulting mixture was extracted with ethyl acetate, washed with brine, dried over anhydrous Na₂SO₄ and concentrated under vacuum. The residue was purified by flash column chromatography on silica gel, eluting with ethyl acetate/petroleum ether (1:5) to give 3-(1-methyl-1H-pyrazol-4-yl)-6-nitro-1-(phenylsulfonyl)-1H-indole (150.2 mg) as a dark grey solid. LCMS Method CB: [M+H]⁺=383.

### Step 4: 3-(1-methyl-1H-pyrazol-4-yl)-6-nitro-1H-indole

3-(1-Methyl-1H-pyrazol-4-yl)-6-nitro-1-(phenylsulfonyl)-1H-indole (500.0 mg, 1.3 mmol, 1.0 equiv.) was dissolved in MeOH/water (5.0 mL/5.0 mL), NaOH (261.0 mg, 6.5 mmol, 5.0 equiv.) was added. The resulting mixture was stirred for overnight at 60° C. under nitrogen atmosphere and then quenched by the addition of water. The pH value was adjusted to 6 with HCl aqueous (4N), then the resulting mixture was extracted with ethyl acetate, washed with brine, dried over anhydrous Na₂SO₄ and concentrated under vacuum to give 3-(1-methyl-1H-pyrazol-4-yl)-6-nitro-1H-indole (100 mg, 31.6%) as a red solid. LCMS Method CB: [M+H]⁺=243.

### Step 5: 3-(1-methyl-1H-pyrazol-4-yl)-1H-indol-6-amine

3-(1-Methyl-1H-pyrazol-4-yl)-6-nitro-1H-indole (100.0 mg, 0.4 mmol, 1.0 equiv.) was dissolved in ethyl acetate (5.0 mL), Pd/C (10% wt., 50.0 mg, 0.05 mmol, 0.1 equiv.) was added. The reaction mixture was degassed and back filled with hydrogen for three times, then stirred for overnight at room temperature under atmosphere of hydrogen. After filtration, the filtrate was concentrated under vacuum to give 3-(1-methyl-1H-pyrazol-4-yl)-1H-indol-6-amine (86.0 mg, 98.2%) as a red solid. LCMS Method CB: [M+H]⁺=213.

### Synthesis of Intermediate A30 (3-(1-methyl-1H-pyrazol-4-yl)-1H-indol-6-amine)

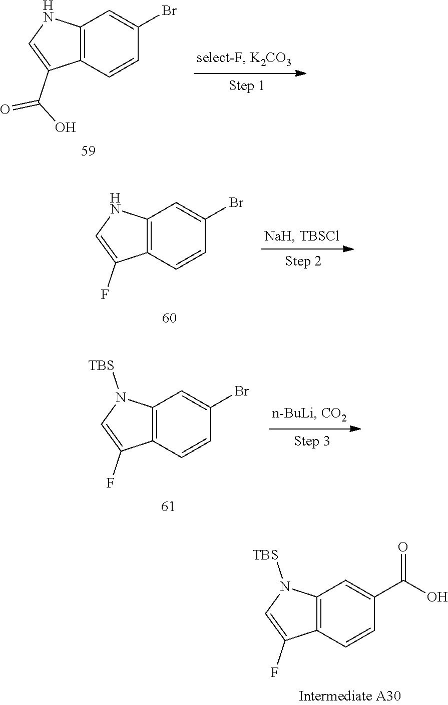

### Step 1: 6-bromo-3-fluoro-1H-indole

6-Bromo-1H-indole-3-carboxylic acid (15.0 g, 62.5 mmol, 1.0 equiv.) and Na₂CO₃ (26.5 g, 249.9 mmol, 4.0 equiv.) were dissolved in DCE/water (80.0 mL/40.0 mL), Select-F (44.3 g, 125.0 mmol, 2.0 equiv.) was added at 0° C. The resulting mixture was stirred for overnight at room temperature under nitrogen atmosphere and quenched by the addition of water under 0° C. The resulting mixture was extracted with DCM, dried over anhydrous Na₂SO₄ and concentrated under vacuum. The residue was purified by flash column chromatography on silica gel, eluting with ethyl acetate/petroleum ether (1:1) to give 6-bromo-3-fluoro-1H-indole (12.0 g, 89.7%) as a yellow solid. LCMS Method CF: [M−H]⁻=212. ¹H NMR (400 MHz, DMSO-d₆): δ 11.01 (s, 1H), 7.59-7.57 (m, 1H), 7.52-7.49 (m, 1H), 7.38 (t, 1H), 7.21-7.17 (m, 1H).

### Step 2: 6-bromo-1-(tert-butyldimethylsilyl)-3-fluoro-1H-indole

6-Bromo-3-fluoro-1H-indole (12.0 g, 56.1 mmol, 1.0 equiv.) was dissolved in THF (120.0 mL), NaH (60% wt in mineral oil, 2.7 g 112.1 mmol, 2.0 equiv.) was added at 0° C. After stirring for 30 min, TBSCl (12.8 g, 84.7 mmol, 1.5 equiv.) was added. The resulting mixture was stirred for 4 hours at room temperature under atmosphere of nitrogen and then quenched by the addition of saturated aqueous NH₄Cl at 0° C. The aqueous layer was extracted with ethyl acetate and concentrated under vacuum. The residue was purified by flash column chromatography on silica gel, eluting with petroleum ether to give 6-bromo-1-(tert-butyldimethylsilyl)-3-fluoroindole (13.1 g, 70.6%) as yellow oil. LCMS Method CB: [M−H]⁻=326.

### Step 3: 1-(tert-butyldimethylsilyl)-3-fluoro-1H-indole-6-carboxylic acid

6-Bromo-1-(tert-butyldimethylsilyl)-3-fluoroindole (12.0 g, 36.6 mmol, 1.0 equiv.) was dissolved in THF (120.0 mL), n-BuLi (2.5 M in hexane, 21.6 mL, 54.0 mmol, 1.5 equiv.) was added dropwise at −78° C. under atmosphere of nitrogen. After stirring for 30 min, CO₂ (gas) was introduced into the solution at −78° C. The final reaction mixture was stirred for additional 1 hour at −78° C. and then quenched by the addition of aqueous NH₄Cl . The mixture was acidified to pH=3 with conc. HCl aqueous. The solution was extracted with ethyl acetate and concentrated under vacuum to give 1-(tert-butyldimethylsilyl)-3-fluoroindole-6-carboxylic acid (10.1 g 93.2%) as a yellow solid. LCMS Method CC: [M−H]⁻=292.

### Synthesis of Intermediate A31 (4-chloro-3-fluoro-1H-indole-6-carboxylic acid)

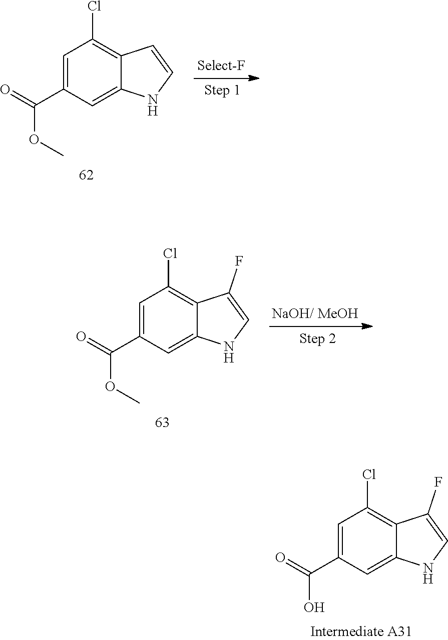

### Step 1: methyl 4-chloro-3-fluoro-1H-indole-6-carboxylate

Methyl 4-chloro-1H-indole-6-carboxylate (100.0 mg, 0.5 mmol, 1.0 equiv.) was dissolved in MeCN (5.0 mL) and water (5.0 mL), NaHCO₃ (80.1 mg, 1.0 mmol, 2.0 equiv.) and Selectfluor (253.5 mg, 0.7 mmol, 1.5 equiv.) were added. The resulting solution was stirred for 16 hours at room temperature and concentrated under vacuum. The residue was purified by flash column chromatography on silica gel, eluting with ethyl acetate/petroleum ether (1:3) to give methyl 4-chloro-3-fluoro-1H-indole-6-carboxylate (40 mg, 36.8%) as a yellow solid. LCMS Method CD: [M+H]⁺=228.

### Step 2: 4-chloro-3-fluoro-1H-indole-6-carboxylic acid

Methyl 4-chloro-3-fluoro-1H-indole-6-carboxylate (100.0 mg, 0.4 mmol, 1.0 equiv.) was dissolved in MeOH/water (5.0 mL/5.00 mL), NaOH (175.7 mg, 4.4 mmol, 10.0 equiv.) was added. The resulting solution was stirred for 5 hours at 50° C. and then quenched by the addition of water. The pH value of the solution was adjusted to 4 with HCl aqueous (6 mol/L). The resulting solution was extracted with ethyl acetate and concentrated under vacuum. The residue was purified by flash column chromatography on silica gel, eluting with ethyl acetate/petroleum ether (1:1) to give 4-chloro-3-fluoro-1H-indole-6-carboxylic acid (60 mg, 63.9%) as a light yellow solid. LCMS Method CD: [M−H]⁻=212.

### Synthesis of Intermediate A32 (4-(3-((1H-1,2,4-triazol-1-yl)methyl)piperidin-1-yl)-1H-indole-6-carboxylic acid)

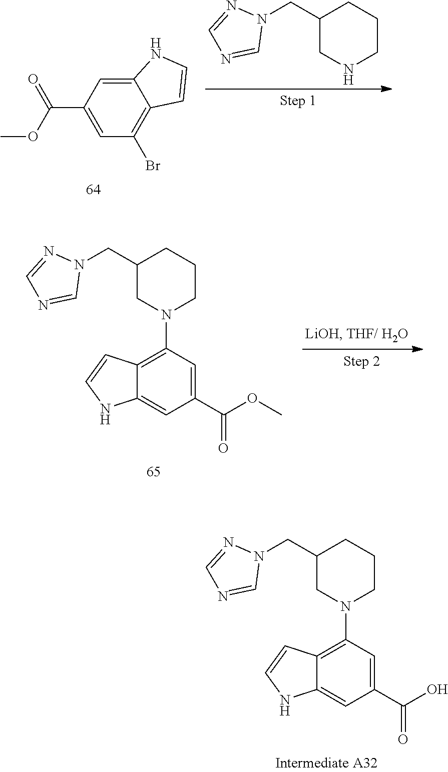

### Step 1: methyl 4-(3-((1H-1,2,4-triazol-1-yl)methyl)piperidin-1-yl)-1H-indole-6-carboxylate

Methyl 4-bromo-1H-indole-6-carboxylate (1.0 g, 3.9 mmol, 1.0 equiv.) was dissolved in dioxane (20.0 mL), Cs₂CO₃ (2.6 g, 7.9 mmol, 2.0 equiv.), 3-((1H-1,2,4-triazol-1-yl)methyl)piperidine (778.5 mg, 4.7 mmol, 1.2 equiv.) and Pd-PEPPSI-IPentCl₂-methylpyridine (o-picoline) (330.2 mg, 0.4 mmol, 0.1 equiv.) were added. The reaction mixture was stirred for 16 hours at 100° C. and quenched by the addition of water. The solution was extracted with ethyl acetate, dried over anhydrous sodium sulfate and concentrated under vacuum. The residue was purified by flash column chromatography on silica gel, eluting with dichloromethane/methanol (95:5) to give methyl 4-(3-((1H-1,2,4-triazol-1-yl)methyl)piperidin-1-yl)-1H-indole-6-carboxylate (700 mg) as a yellow solid. LCMS Method CA: [M+H]⁺=340.

### Step 2: 4-(3-((1H-1,2,4-triazol-1-yl)methyl)piperidin-1-yl)-1H-indole-6-carboxylic acid

Methyl 4-(3-((1H-1,2,4-triazol-1-yl)methyl)piperidin-1-yl)-1H-indole-6-carboxylate (620.0 mg, 1.8 mmol, 1.0 equiv.) was dissolved in MeOH (5.0 mL), a solution of NaOH in water (2M, 5 mL, 10.0 mmol, 5.0 equiv.) was added. The reaction mixture was stirred for 15 hours at room temperature. The pH value of the solution was adjusted to 4 with HCl aqueous (1 mol/L) and the mixture solution was extracted with ethyl acetate and concentrated under vacuum. The residue was purified by flash column chromatography on silica gel, eluting with dichloromethane/methanol (10:1) to give 4-(3-((1H-1,2,4-triazol-1-yl)methyl)piperidin-1-yl)-1H-indole-6-carboxylic acid (300 mg) as a yellow solid. LCMS Method CA: [M+H]⁺=326.

### Synthesis of Intermediate A33 (6-amino-N,N-dimethyl-1H-indole-4-carboxamide)

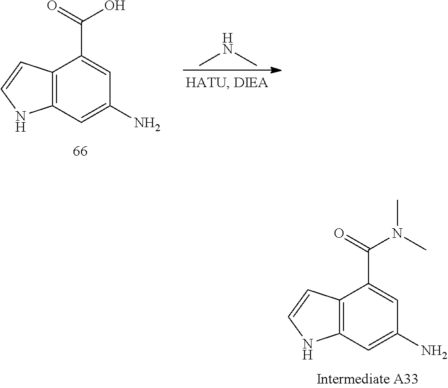

6-amino-1H-indole-4-carboxylic acid (100.0 mg, 0.6 mmol, 1.0 equiv.) and HATU (260.0 mg, 0.7 mmol, 1.2 equiv.) were dissolved in THF (20.0 mL), DIEA (0.4 mL, 2.4 mmol, 4 equiv.) and dimethylamine hydrogen chloride (145 mg, 1.8 mmol, 3.0 equiv.) were added. The resulting mixture was stirred for 4 hours and then concentrated under vacuum. The residue was purified by flash column chromatography on silica gel, eluting with dichloromethane/methanol (10:1) to give 6-amino-N,N-dimethyl-1H-indole-4-carboxamide (90 mg, 78.0%) as a white solid. LCMS Method CF: [M+H]⁺=204.

### Synthesis of Intermediate A34 (4-((dimethylamino)methyl)-1H-indol-6-amine)

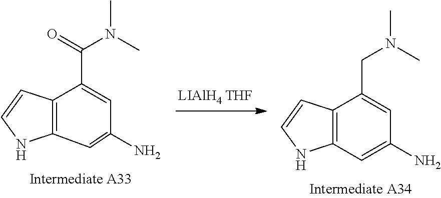

6-Amino-N,N-dimethyl-1H-indole-4-carboxamide (100.0 mg, 0.5 mmol, 1.0 equiv.) was dissolved in THF (5.0 mL), LiAlH₄ (14.9 mg, 0.4 mmol, 4.0 equiv.) was added at 0° C. The reaction mixture was stirred for 2 hours at room temperature and then quenched by the addition of aqueous NaOH (10%) at 0° C. After concentration and washing the filtrate cake with ethyl acetate, the filtrate was concentrated under vacuum to give 4-((dimethylamino)methyl)-1H-indol-6-amine (61 mg, 65.5%) as yellow solid. LCMS Method CD: [M+H]⁺=190.

### Synthesis of Intermediate A35 (2-chloro-6-methylpyridin-4-yl)methanamine)

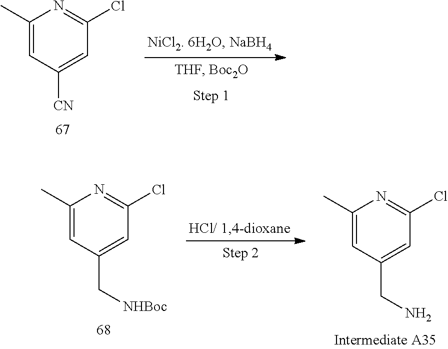

### Step 1: tert-butyl N-[(2-chloro-6-methylpyridin-4-yl)methyl]carbamate

2-Chloro-6-methylpyridine-4-carbonitrile (200.0 mg, 1.3 mmol, 1.0 equiv.) was dissolved in THF (10.0 mL), NiCl₂.6H₂O (31.2 mg, 0.1 mmol, 0.1 equiv.) and Boc₂O (572.2 mg, 2.6 mmol, 2.0 equiv.) was added. This was followed by the addition of NaBH₄ (59.5 mg, 1.6 mmol, 1.2 equiv.) under 0° C. The resulting solution was stirred for 8 hours at room temperature and concentrated under vacuum. The residue was purified by flash column chromatography on silica gel, eluting with ethyl acetate/petroleum ether (1:3) to give tert-butyl N-[(2-chloro-6-methylpyridin-4-yl)methyl]carbamate (100 mg, 29.7%) as a off-white solid. LCMS Method CD: [M+H]⁺=257.

### Step 2: (2-chloro-6-methylpyridin-4-yl)methanamine

tert-Butyl N-[(2-chloro-6-methylpyridin-4-yl)methyl]carbamate (100.0 mg, 0.4 mmol, 1.0 equiv.) was dissolved in HCl/1,4-dioxane (4M, 10.0 mL). The resulting solution was stirred for 5 hours at room temperature and quenched by the addition of water. The pH value of the solution was adjusted to 8 with saturated aqueous Na₂CO₃ solution, then extracted with DCM, washed with brine and concentrated under vacuum to give 1-(2-chloro-6-methylpyridin-4-yl)methanamine (50 mg, 73.1%) as a off-white solid. LCMS Method CD: [M+H]⁺=157.

### Synthesis of Intermediate B1 (6-isothiocyanato-1H-indole)

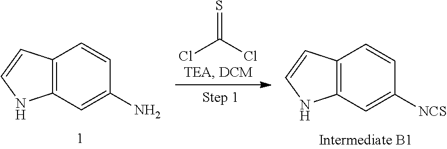

1H-indol-6-amine (1.0 g, 7.6 mmol, 1.0 equiv.) was dissolved in THF (30.0 mL), TEA (2.1 mL, 15.1 mmol, 2.0 equiv.) and thiophosgene (1.7 g, 15.1 mmol, 2.0 equiv.) were added. The resulting solution was stirred for 2 hours at ambient temperature and then diluted with 100 mL of water. The resulting solution was extracted with ethyl acetate, dried over anhydrous sodium sulfate and concentrated under vacuum to result in 6-isothiocyanato-1H-indole (1.2 g, 91.0%) as a dark yellow solid. Synthesis of intermediate B2 (6-isothiocyanato-7-methyl-1H-indole)

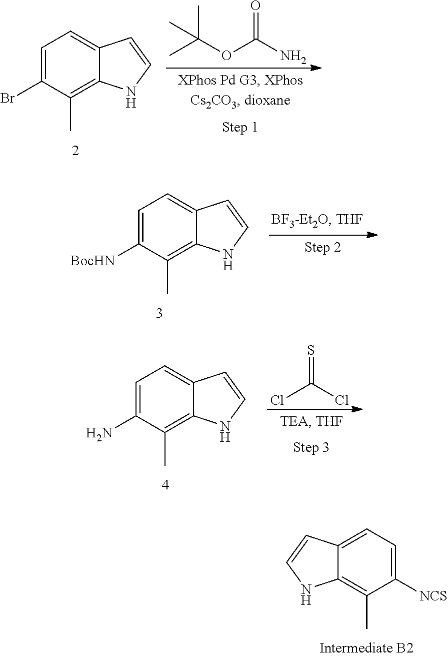

### Step 1: tert-butyl (7-methyl-1H-indol-6-yl)carbamate

6-Bromo-7-methyl-1H-indole (400.0 mg, 1.9 mmol, 1.0 equiv.) was dissolved in dioxane (5.0 mL), tert-butyl carbamate (334.6 mg, 2.9 mmol, 1.5 equiv.), Cs₂CO₃ (1240.8 mg, 3.8 mmol, 2.0 equiv.), XPhos Pd G3 (322.3 mg, 0.4 mmol, 0.2 equiv.) and XPhos (181.5 mg, 0.4 mmol, 0.2 equiv.) were added under nitrogen. The resulting solution was stirred for 16 hours at 90° C. and concentrated under vacuum. The residue was purified by flash column chromatography on silica gel, eluting with ethyl acetate/petroleum ether (1/5) to give tert-butyl N-(7-methyl-1H-indol-6-yl)carbamate (250 mg, 53.3%) was isolated as a light yellow solid. LCMS Method CC: [M−H]⁻=245.

### Step 2: 7-methyl-1H-indol-6-amine

tert-butyl N-(7-methyl-1H-indol-6-yl)carbamate (200.0 mg, 0.8 mmol, 1.0 equiv.) was dissolved in THF (15.0 mL), BF₃.Et₂O (1.00 mL, 8.0 mmol, 10.0 equiv.) was added. The resulting solution was stirred for 30 min at ambient temperature and then concentrated under vacuum to give 7-methyl-1H-indol-6-amine (110 mg, 92.7%) as a light yellow solid. LCMS Method CA: [M+H]⁺=147.

### Step 3: 6-isothiocyanato-7-methyl-1H-indole

7-Methyl-1H-indol-6-amine (110.0 mg, 0.8 mmol, 1.0 equiv.) was dissolved in THF (20.0 mL), TEA (0.2 mL, 1.5 mmol, 2.0 equiv.) and thiophosgene (173.0 mg, 1.5 mmol, 2.0 equiv.) were added. The resulting solution was stirred for 30 min at ambient temperature and then diluted with 50 mL of water. The resulting solution was extracted with ethyl acetate, dried over anhydrous sodium sulfate and concentrated under vacuum to give 6-isothiocyanato-7-methyl-1H-indole (100 mg, 70.6%) as a light yellow solid.

### Synthesis of Intermediate B3 (4-chloro-6-isothiocyanato-1H-indole)

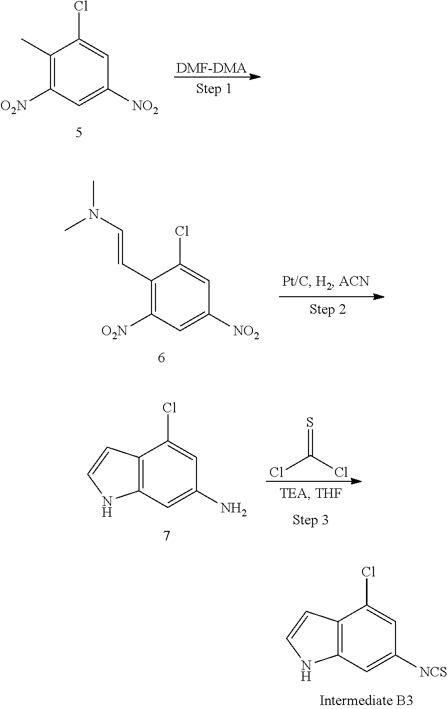

### Step 1: (E)-2-(2-chloro-4,6-dinitrophenyl)-N,N-dimethylethen-1-amine

1-chloro-2-methyl-3,5-dinitrobenzene (2.0 g, 9.2 mmol, 1.0 equiv.) was dissolved in DMF (20.0 mL), DMF-DMA (4.4 g, 36.9 mmol, 4.0 equiv.) were added. The reaction mixture was stirred for 4 hours at 80° C. and then diluted with water. The solution was extracted with ethyl acetate, dried over anhydrous sodium sulfate and concentrated under vacuum to give [(E)-2-(2-chloro-4,6-dinitrophenyl)ethenyl]dimethylamine (1.8 g, 71.7%) as a white solid. LCMS Method CA: [M+H]⁺=272.

### Step 2: 4-chloro-1H-indol-6-amine

[(E)-2-(2-chloro-4,6-dinitrophenyl)ethenyl]dimethylamine (1.8 g, 6.6 mmol, 1.0 equiv.) was dissolved in ACN (20.0 mL), Pt/C (200 mg, 10% wet., 0.0.1 mmol, 0.02 equiv.) were added under N2 atmosphere. The reaction mixture was degassed and back filled with hydrogen for three times and stirred for 16 hours at room temperature under atmosphere of hydrogen. The reaction mixture was filtered through Celite and the filtrate was concentrated under vacuum to give 4-chloro-1H-indol-6-amine (700 mg, 63.4%) as a light yellow solid. LCMS Method CB: [M+H]⁺=167.

### Step 3: 4-chloro-6-isothiocyanato-1H-indole

4-chloro-1H-indol-6-amine (700.0 mg, 4.2 mmol, 1.0 equiv.) was dissolved in THF (20.0 mL), TEA (1.2 mL, 8.4 mmol, 2.0 equiv), thiophosgene (966.1 mg, 8.4 mmol, 2.0 equiv) were added and the reaction mixture was stirred for 2 hours at ambient temperature. The reaction mixture was concentrated under vacuum to give 4-chloro-6-isothiocyanato-1H-indole (500 mg, 57.0%) as a white solid.

### Synthesis of Intermediate B4 (6-isothiocyanato-4-methyl-1H-indole)

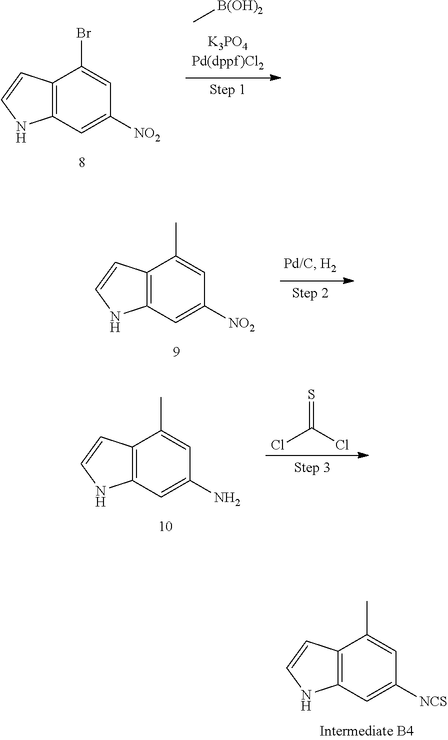

### Step 1: 4-methyl-6-nitro-1H-indole

4-Bromo-6-nitro-1H-indole (1.0 g, 4.1 mmol, 1.0 equiv.) was dissolved in dioxane/water (5.0 mL/1.0 mL), methylboronic acid (0.5 g, 8.3 mmol, 2.0 equiv.), K₃PO₄ (1.8 g, 8.3 mmol, 2.0 equiv.) and Pd(dppf)Cl₂ (0.3 g, 0.4 mmol, 0.1 equiv.) were added under nitrogen. The resulting solution was stirred for 4 hours at 90° C. and then concentrated under vacuum. The residue was purified by flash column chromatography on silica gel, eluting with ethyl acetate/petroleum ether (1/3) to give 4-methyl-6-nitro-1H-indole (540 mg, 73.9%) as a light yellow solid. LCMS Method CA: [M+H]⁺=177.

### Step 2: 4-methyl-1H-indol-6-amine

4-Methyl-6-nitro-1H-indole (400.0 mg, 2.3 mmol, 1.0 equiv.) was dissolved in MeOH (30.0 mL), Pd/C (50.0 mg, 0.5 mmol, 0.2 equiv.) was added under nitrogen. The solution was degassed and back filled with hydrogen for three times, and then stirred for 3 hours at ambient temperature under atmosphere of hydrogen. The solids were filtered out and the filtrate was concentrated under vacuum to give crude 4-methyl-1H-indol-6-amine (350 mg) as a light yellow solid. LCMS Method CA: [M+H]⁺=147.

### Step 3: 6-isothiocyanato-4-methyl-1H-indole

4-Methyl-1H-indol-6-amine (340.0 mg, 2.3 mmol, 1.0 equiv.) was dissolved in THF (20.0 mL), TEA (0.6 mL, 4.7 mmol, 2.0 equiv.), thiophosgene (510.0 mg, 4.4 mmol, 2.0 equiv.) were added. The resulting solution was stirred for 1 hour at ambient temperature and then quenched by the addition of water. The resulting solution was extracted with ethyl acetate, dried over anhydrous sodium sulfate and concentrated under vacuum to give 260 mg of 6-isothiocyanato-4-methyl-1H-indole as a light yellow solid.

### Synthesis of Intermediate B5 (7-fluoroquinoline)

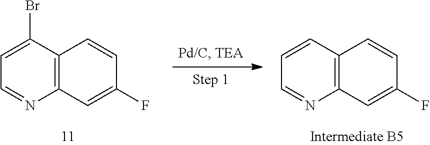 [SMILES: Fc1ccc2c(Br)ccnc2c1 ; Fc1ccc2cccnc2c1]

4-Bromo-7-fluoroquinoline (1.0 g, 4.4 mmol, 1.0 equiv.) was dissolved in MeOH (20.0 mL), then Pd/C (100 mg, 10% wet, 0.1 mmol, 0.02 equiv.) and TEA (1.2 mL, 8.8 mmol, 2.0 equiv.) were added. The resulting mixture was stirred for 2 hours at room temperature and concentrated under vacuum. The residue was purified by flash column chromatography on silica gel, eluting with acetate/petroleum ether (1:1) to give 7-fluoroquinoline (580 mg, 89.1%) as a white solid. LCMS Method CA: [M+H]⁺=148.

### Synthesis of Intermediate B6 (6-chloro-7-(trifluoromethyl)quinoline) and intermediate B7 (6-chloro-5-(trifluoromethyl)quinoline)

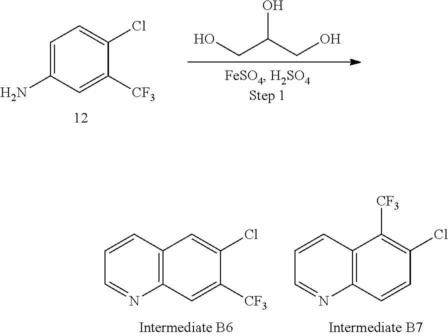 [SMILES: Nc1ccc(Cl)c(C(F)(F)F)c1 ; OCC(O)CO ; FC(F)(F)c1cc2ncccc2cc1Cl ; FC(F)(F)c1c(Cl)ccc2ncccc12]

4-chloro-3-(trifluoromethyl)aniline (10.0 g, 51.1 mmol, 1.0 equiv.) was dissolved in glycerol (16 mL), then FeSO₄ (3.2 g, 21.4 mmol, 0.4 equiv.) wad added. This was followed by the addition of conc. H₂SO₄ (9.5 mL, 97.2 mmol, 3.5 equiv.) dropwise at 0° C. The reaction mixture was stirred for 16 hours at 140° C. After cooled to 0° C., the pH value of the solution was adjusted to 11 with NaOH (aq.). The solids were filtered out and the filtrate was diluted with of ethyl acetate. The solution was washed with brine, dried over anhydrous sodium sulfate and concentrated under vacuum. The residue was purified by flash column chromatography on silica gel, eluting with acetate/petroleum ether (1:2) to give a mixture 6-chloro-7-(trifluoromethyl)quinolone and 6-chloro-5-(trifluoromethyl)quinoline (5:1, 1.1 g, 9.3%) as a brown crude solid. LCMS Method CD: [M+H]⁺=232.

The intermediates in the following table were prepared using the same method described for Intermediates B6-B7.

| Intermediate | Starting material Used | Structure | LCMS data |
|---|---|---|---|
| Intermediate B8 | 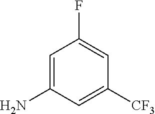 [SMILES: Nc1cc(F)cc(C(F)(F)F)c1] | 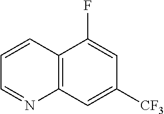 [SMILES: Fc1cc(C(F)(F)F)cc2ncccc12] | Method CA: MS-ESI: 216 [M + H]⁺ |
| Intermediate B9 | 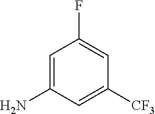 [SMILES: Nc1cc(F)cc(C(F)(F)F)c1] | 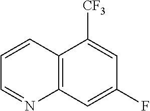 [SMILES: Fc1cc(C(F)(F)F)c2cccnc2c1] | Method CA: MS-ESI: 216 [M + H]⁺ |
| Intermediate B10 | 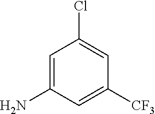 [SMILES: Nc1cc(Cl)cc(C(F)(F)F)c1] | 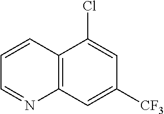 [SMILES: FC(F)(F)c1cc(Cl)c2cccnc2c1] | Method CD: MS-ESI: 232 [M + H]+ |
| Intermediate B11 | 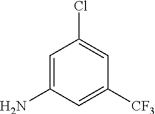 [SMILES: Nc1cc(Cl)cc(C(F)(F)F)c1] | 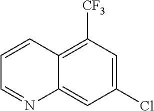 [SMILES: FC(F)(F)c1cc(Cl)cc2ncccc12] | Method CD: MS-ESI: 232 [M + H]+ |

### Synthesis of Intermediate B12 (3-methyl-7-(trifluoromethyl)quinoline)

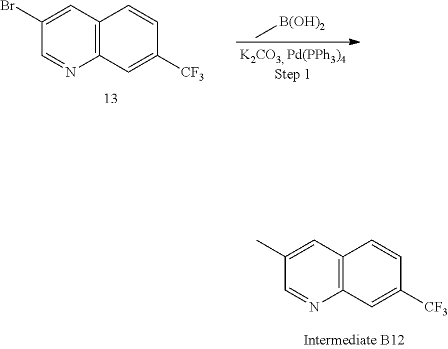

3-Bromo-7-(trifluoromethyl)quinoline (600.0 mg, 2.1 mmol, 1.0 equiv.) was dissolved in DMSO/H₂O (10 mL/1 mL), then methylboronic acid (390.3 mg, 6.5 mmol, 3.0 equiv.), Pd(PPh₃)₄(502.3 mg, 0.4 mmol, 0.2 equiv.) and K₂CO₃ (1.5 g, 10.8 mmol, 5.0 equiv.) were added under atmosphere of nitrogen. The reaction mixture was stirred for 8 hours at 80° C. under atmosphere of nitrogen and then concentrated under vacuum. The residue was purified by prep-TLC (ethyl acetate/petroleum ether=1:6) to give 3-methyl-7-(trifluoromethyl)quinolone (430 mg, 93.6%) as a white solid. LCMS Method CE: [M+H]⁺=212.

### Synthesis of Intermediate B13 (1-((2-(trimethylsilyl)ethoxy)methyl)-1H-indol-6-amine)

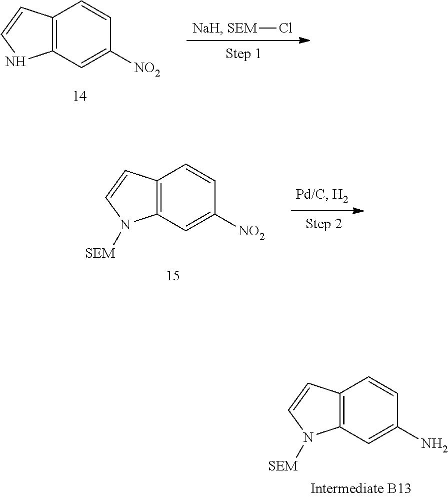

### Step 1: 6-nitro-1-((2-(trimethylsilyl)ethoxy)methyl)-1H-indole

6-Nitro-1H-indole (5.0 g, 30.8 mmol, 1.0 equiv.) was added in THF (50.0 mL), NaH (60% wt in mineral oil, 2.4 g, 61.6 mmol, 2.0 equiv.) was added in portions under atmosphere of nitrogen. After stirring for 30 min, SEMCl (7.7 g, 46.3 mmol, 1.5 equiv.) was added and the mixture was stirred for additional 16 hours at ambient temperature, then quenched by the addition of water. The resulting mixture was extracted with ethyl acetate, washed with brine, dried over anhydrous sodium sulfate and concentrated under reduced pressure. The residue was purified by flash column chromatography on silica gel, eluting with acetate/petroleum ether (1:5) to give 6-Nitro-1-[[2-(trimethylsilyl)ethoxy]methyl]indole (8.9 g, 98.7%) as a yellow liquid.

### Step 2: 1-((2-(trimethylsilyl)ethoxy)methyl)-1H-indol-6-amine

6-Nitro-1-[[2-(trimethylsilyl)ethoxy]methyl]indole (5.0 g, 17.1 mmol, 1.0 equiv.) was dissolved in MeOH (20.0 mL), Pd/C (200 mg, 10% wet, 1.9 mmol, 0.1 equiv.) was added under atmosphere of nitrogen. The resulting mixture was degassed and back filled with hydrogen for three times, then stirred for 16 hours at room temperature under atmosphere of hydrogen. The resulting mixture was filtered through a Celite pad, and the filtrate was concentrated under reduced pressure to give 1-((2-(trimethylsilyl)ethoxy)methyl)-1H-indol-6-amine (4.2 g, 93.6%) as a brown yellow oil. LCMS Method CD: [M+H]⁺=263.

The intermediates in the following table were prepared using the same method described for Intermediate B13.

| Intermediate | Starting material Used | Structure | LCMS data |
|---|---|---|---|
| Intermediate B14 | 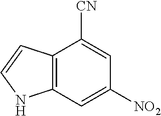 [SMILES: N#Cc1cc([N+](=O)[O-])cc2[nH]ccc12] | 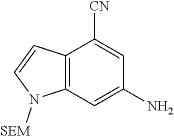 [SMILES: C[Si](C)(C)CCOCn1ccc2c(C#N)cc(N)cc21] | Method CF: MS-ESI: 287 [M + H]⁺ |
| Intermediate B15 | 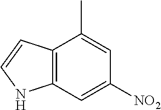 | 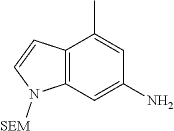 [SMILES: Cc1cc(N)cc2c1ccn2COCC[Si](C)(C)C] | Method CA: MS-ESI: 277 [M + H]⁺ |
| Intermediate B16 | 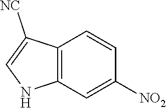 [SMILES: N#Cc1c[nH]c2cc([N+](=O)[O-])ccc12] | 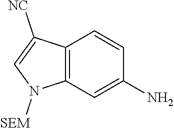 [SMILES: C[Si](C)(C)CCOCn1cc(C#N)c2ccc(N)cc21] | Method CA: MS-ESI: 287 [M + H]⁺ |

### Synthesis of Intermediate B17 (6-bromo-1-((2-(trimethylsilyl)ethoxy)methyl)-1H-indole)

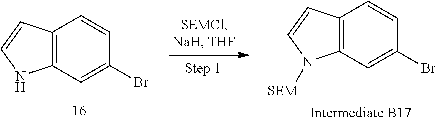 [SMILES: Brc1ccc2cc[nH]c2c1 ; C[Si](C)(C)CCOCn1ccc2ccc(Br)cc21]

To a stirred mixture of 6-bromo-1H-indole (5.0 g, 25.5 mmol, 1.0 equiv.) in THF (50.0 mL), was added NaH (60% wt in mineral oil, 2.0 g, 51.0 mmol, 2.0 equiv.) in portions at 0° C. under nitrogen. After stirred for 30 min at ambient temperature under nitrogen atmosphere, SEM-Cl (8.50 g, 51.0 mmol, 2.0 equiv.) was added dropwise at 0° C. The resulting mixture was stirred for overnight at ambient temperature and then quenched by the addition of water at 0° C. The solution was extracted with ethyl acetate, dried over anhydrous sodium sulfate and concentrated under vacuum. The residue was purified by flash column chromatography on silica gel, eluting with acetate/petroleum ether (1:8) to give crude 6-bromo-1-((2-(trimethylsilyl)ethoxy)methyl)-1H-indole (8 g, 96.1%) as a red oil. LCMS Method CD: [M+H]⁺=326.

### Synthesis of Intermediate B18 (2-chloro-7-ethylquinoline)

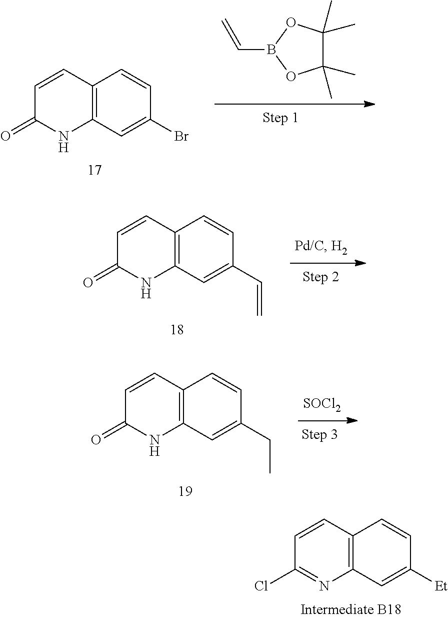

### Step 1: 7-ethenyl-1H-quinolin-2-one

7-Bromo-1H-quinolin-2-one (300.0 mg, 1.3 mmol, 1.0 equiv.) was dissolved in dioxane/water (10.0 mL/0.5 mL), Pd(dppf)Cl₂ (98.0 mg, 0.1 mmol, 0.1 equiv.), K₃PO₄ (852.6 mg, 4.0 mmol, 3.0 equiv.) and 2-ethenyl-4,4,5,5-tetramethyl-1,3,2-dioxaborolane (412.5 mg, 2.7 mmol, 2.0 equiv.) were added under nitrogen. The resulting solution was stirred for 3 hours at 90° C. and then concentrated under vacuum. The residue was purified by flash column chromatography on silica gel, eluting with acetate/petroleum ether (1:4) to give 7-ethenyl-1H-quinolin-2-one (190 mg, 82.9%) as a yellow solid. LCMS Method CA: [M+H]⁺=172.

### Step 2: 7-ethyl-1H-quinolin-2-one

7-Ethenyl-1H-quinolin-2-one (190.0 mg, 1.1 mmol, 1.0 equiv.) was dissolved in MeOH (10.0 mL), Pd/C (23.6 mg, 0.2 mmol, 0.2 equiv) was added under nitrogen. The mixture was degassed and back filled with hydrogen for three times and then stirred for 3 hours at ambient temperature. The solids were filtered out and the filtrate was concentrated under vacuum to give 7-ethyl-1H-quinolin-2-one (180 mg, 93.6%) as yellow oil. LCMS Method CA: [M+H]⁺=174.

### Step 3: 2-chloro-7-ethylquinoline

7-Ethyl-1H-quinolin-2-one (180.0 mg, 1.0 mmol, 1.0 equiv.) was dissolved in DCM (5.0 mL), DMF (0.01 mL, 0.1 mmol, 0.1 equiv.) was added. This was followed by the addition of SOCl₂ (0.25 mL, 3.3 mmol, 3.3 equiv.) dropwise with stirring at 0° C. The resulting solution was stirred for 16 hours at 50° C. and then concentrated under vacuum. The residue was purified by flash column chromatography on silica gel, eluting with acetate/petroleum ether (1:8) to give 2-chloro-7-ethylquinoline (171 mg, 85.9%) as a yellow solid. LCMS Method CA: [M+H]⁺=192.

### Synthesis of Intermediate B19 (2-chloro-7-cyclobutylquinoline)

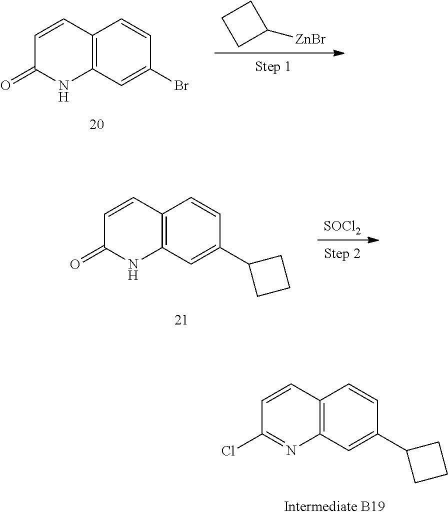

### Step 1: 7-cyclobutylquinolin-2(1H)-one

7-Bromo-1H-quinolin-2-one (440.0 mg, 2.0 mmol, 1.0 equiv.) was dissolved in THF (10.00 mL), bromo(cyclobutyl)zinc (6.0 mL, 3.0 mmol, 1.5 equiv., 0.5 mol/L), CuI (74.8 mg, 0.4 mmol, 0.2 equiv.), Pd(dppf)Cl₂ (143.7 mg, 0.2 mmol, 0.1 equiv.) were added under nitrogen. The resulting solution was stirred for 4 hours at 70° C. and concentrated under vacuum. The residue was purified by flash column chromatography on silica gel, eluting with acetate/petroleum ether (1:2) to give 7-cyclobutylquinolin-2(1H)-one (350 mg, 89.5%) as a light yellow solid. LCMS Method CA: [M+H]⁺=200.

### Step 2: 2-chloro-7-cyclobutylquinoline

7-Cyclobutylquinolin-2(1H)-one (330.0 mg, 1.7 mmol, 1.0 equiv.) was dissolved in DCM (20.0 mL), SOCl₂ (0.25 mL, 3.3 mmol, 2.0 equiv.), DMF (0.01 mL, 0.1 mmol, 0.1 equiv.) were added at 0° C. The resulting solution was stirred for 4 hours at ambient temperature and then quenched by the addition of water. The resulting solution was extracted with DCM, washed with brine, dried over anhydrous sodium sulfate and concentrated under vacuum. The residue was purified by flash column chromatography on silica gel, eluting with acetate/petroleum ether (1:4) to give 2-chloro-7-cyclobutylquinoline (90 mg, 25.0%) as a light yellow solid. LCMS Method CA: [M+H]⁺=218.

### Synthesis of Intermediate B20 (I-methyl-1H-indol-6-amine)

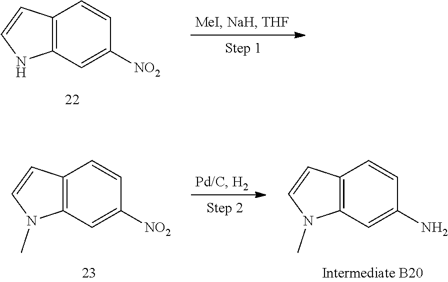

### Step 1: 1-methyl-6-nitro-1H-indole

6-Nitro-1H-indole (500.0 mg, 3.1 mmol, 1.0 equiv.) was dissolved in THF (20.0 mL), NaH (60% wt in mineral oil, 246.7 mg, 6.167 mmol, 2 equiv.) was added under nitrogen. After stirred for 30 min, CH₃I (875.4 mg, 6.2 mmol, 2.0 equiv.) was added. The resulting solution was stirred for 2 hours at ambient temperature and then quenched by the addition of water. The resulting solution was extracted with ethyl acetate, dried over anhydrous sodium sulfate and concentrated under vacuum to result in 1-methyl-6-nitro-1H-indole (500 mg, 92.0%) as a light yellow solid. LCMS Method CA: [M+H]⁺=177.

### Step 2: 1-methyl-1H-indol-6-amine

1-Methyl-6-nitro-1H-indole (250.0 mg, 1.4 mmol, 1.0 equiv.) was dissolved in MeOH (20.0 mL), Pd/C (15.1 mg, 0.1 mmol, 0.1 equiv.) was added under nitrogen. The mixture was degassed and back filled with hydrogen for three times, then stirred for 2 hours at ambient temperature. The solids were filtered out and the filtrate was concentrated under vacuum to give crude 1-methyl-1H-indol-6-amine (200 mg, 96.4%) as a light yellow solid. LCMS Method CA: [M+H]⁺=147.

### Synthesis of Intermediate B21 (6-(trifluoromethyl)isoquinolin-3-amine)

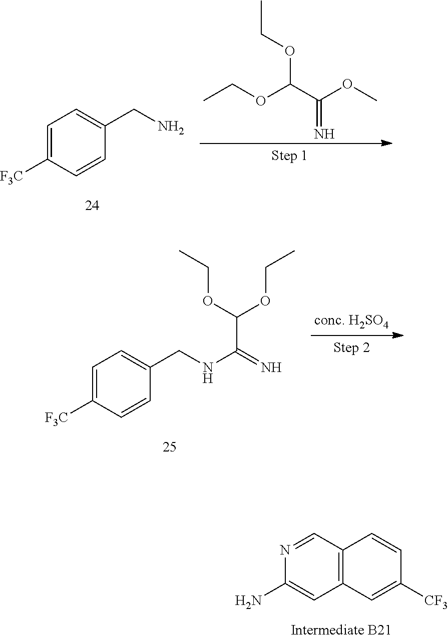 [SMILES: NCc1ccc(C(F)(F)F)cc1 ; CCOC(OCC)C(=N)OC ; CCOC(OCC)C(=N)NCc1ccc(C(F)(F)F)cc1 ; Nc1cc2cc(C(F)(F)F)ccc2cn1]

### Step 1: 2,2-diethoxy-N-(4-(trifluoromethyl)benzyl)acetimidamide

1-[4-(Trifluoromethyl)phenyl]methanamine (1.0 g, 5.7 mmol, 1.0 equiv.) was dissolved in MeOH (30.0 mL), methyl 2,2-diethoxyethanimidate (1.8 g, 11.4 mmol, 2.0 equiv.) was added. The resulting solution was stirred for 4 hours at ambient temperature and concentrated under vacuum to give 2,2-diethoxy-N-[[4-(trifluoromethyl)phenyl]methyl]ethanimidamide (2.1 g) as a yellow crude solid. The crude product was used directly in the next step without any purification. LCMS Method CH: [M+H]⁺=305.

### Step 2: 6-(trifluoromethyl)isoquinolin-3-amine

2,2-diethoxy-N-[[4-(trifluoromethyl)phenyl]methyl]ethanimidamide (2.1 g, 6.9 mmol, 1.0 equiv.) was dissolved in conc. H₂SO₄ (15.0 mL). The resulting solution was stirred for 2 hours at 50° C. and then quenched with water/ice. The pH value of the solution was adjusted to 7 with aq. NaOH (20%). The resulting solution was extracted with ethyl acetate, dried over anhydrous sodium sulfate and concentrated under vacuum. The residue was purified by flash column chromatography on silica gel, eluting with acetate/petroleum ether (1:1) to give 6-(trifluoromethyl)isoquinolin-3-amine (200 mg, 13.7%) as a light yellow solid. LCMS Method CA: [M+H]⁺=213. ¹H NMR (400 MHz, DMSO-d₆): δ 8.91 (d, 1H), 7.99-7.96 (m, 2H), 7.30-7.27 (m, 1H), 6.75 (s, 1H).

### Synthesis of Intermediate B22 ((E)-2-bromo-6-(but-1-en-1-yl)pyridine)

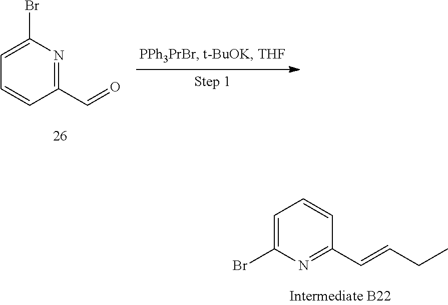

To a stirred solution 6-bromopyridine-2-carbaldehyde (500.0 mg, 2.7 mmol, 1.0 equiv.) and bromotriphenyl(propyl)-15-phosphane (2.1 g, 5.4 mmol, 2.0 equiv.) in THF (20.0 mL) was added t-BuOK (904.9 mg, 8.1 mmol, 3.0 equiv.) in portions at 0° C. under nitrogen. The resulting mixture was stirred for 5 hours at ambient temperature under atmosphere of nitrogen and then quenched by the addition of water. The solution was extracted with ethyl acetate and concentrated under vacuum. The residue was purified by flash column chromatography on silica gel, eluting with acetate/petroleum ether (1:5) to give (E)-2-bromo-6-(but-1-en-1-yl)pyridine (250 mg, 37.7%) as a dark yellow oil. LCMS Method CA: [M+H]⁺=212.

### Example 1. Synthesis of 3-(3-cyano-1H-indol-6-yl)-1-[4-(trifluoromethyl)phenyl]urea (Compound 258)

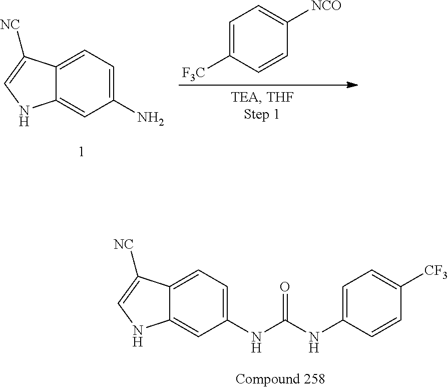

To a solution of 6-amino-1H-indole-3-carbonitrile (100.0 mg, 0.6 mmol, 1.0 equiv) and 1-isocyanato-4-(trifluoromethyl)benzene (119.0 mg, 0.6 mmol, 1.0 equiv) in THF (10 mL), was added TEA (128.8 mg, 1.3 mmol, 2.0 equiv). The resulting mixture was stirred for overnight at room temperature and then quenched by the addition of water. The solution was extracted with EtOAc, and organic layer dried over anhydrous Na₂SO₄ and concentrated under vacuum. The residue was purified by silica gel column chromatography, eluting with PE/EtOAc (5:1) to give 3-(3-cyano-1H-indol-6-yl)-1-[4-(trifluoromethyl)phenyl]urea (97.6 mg, 43.7%) as a off-white solid. LCMS Method AA: [M+H]⁺=345. ¹H NMR (300 MHz, DMSO-d₆) δ 12.04 (s, 1H), 9.08 (s, 1H), 8.90 (s, 1H), 8.15 (s, 1H), 7.97 (s, 1H), 7.69-7.64 (m, 4H), 7.53 (d, J=8.4 Hz, 1H), 7.12-7.09 (m, 1H).

### Example 2: Synthesis of 3-(3-fluoro-1H-indol-6-yl)-1-[4-(trifluoromethyl)phenyl]urea (Compound 242)

### 1. Synthesis of 6-bromo-3-fluoro-1H-indole

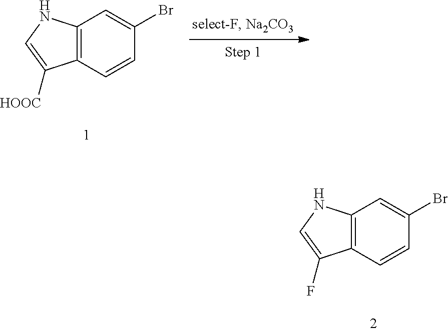 [SMILES: O=C(O)c1c[nH]c2cc(Br)ccc12 ; Fc1c[nH]c2cc(Br)ccc12]

To a solution of 6-bromo-1H-indole-3-carboxylic acid (5.0 g, 20.8 mmol, 1.0 equiv) in DCE (40.0 mL) and H₂O (20.0 mL), was added Select-F (14.8 g, 41.7 mmol, 2.0 equiv) and Na₂CO₃ (8.8 g, 83.3 mmol, 4.0 equiv) at 0° C. The resulting solution was stirred for 12 hours at room temperature and then quenched by the addition of water. The resulting solution was extracted with dichloromethane and the organic layer was separated and concentrated under vacuum. The residue was applied onto a silica gel column, eluting with ethyl acetate/petroleum ether (1:1) to give 6-bromo-3-fluoro-1H-indole (2 g, 44.9%) as a yellow solid. LCMS Method AB: [M−H]⁻=212

### 2. Synthesis of methyl 3-fluoro-1H-indole-6-carboxylate

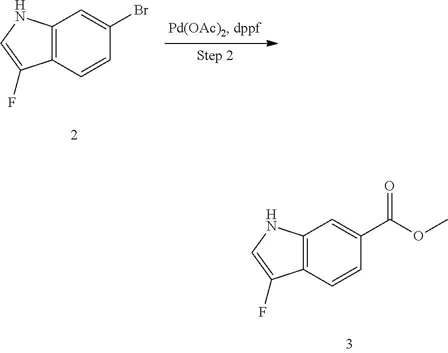

To a solution of 6-bromo-3-fluoro-1H-indole (1.0 g, 4.7 mmol, 1.0 equiv) in MeOH (5.0 mL) and DMSO (5.0 mL) were added Pd(OAc)₂ (0.2 g, 0.9 mmol, 0.2 equiv), Dppf (0.8 g, 1.4 mmol, 0.3 equiv), and TEA (1.0 g, 9.3 mmol, 2.0 equiv). The resulting solution was stirred for 12 hours at 100° C. under atmosphere of CO in a high pressure reaction vessel. After cooling, the resulting mixture was concentrated under vacuum. The residue was applied onto a silica gel column with ethyl acetate/petroleum ether (1:2) to give methyl 3-fluoro-1H-indole-6-carboxylate (300 mg, 33.2%) as yellow oil. LCMS Method AB: [M+H]⁺=194

### 3. Synthesis of 3-fluoro-1H-indole-6-carboxylic acid

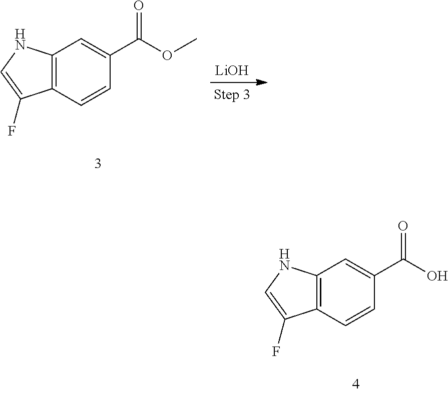

To a solution of methyl 3-fluoro-1H-indole-6-carboxylate (300.0 mg, 1.6 mmol, 1.0 equiv) in water (2.0 mL) and MeOH (2.0 mL), LiOH (148.8 mg, 6.2 mmol, 4.0 equiv) was added. The resulting solution was stirred for 2 hr at room temperature. The pH value of the solution was adjusted to 5 with HCl (4 mol/L). The resulting solution was extracted with ethyl acetate and the organic layers were concentrated under vacuum to give 3-fluoro-1H-indole-6-carboxylic acid (150 mg, 53.9%) as a yellow solid. LCMS Method AB: [M−H]⁻=178

### 4. Synthesis of 3-fluoro-1H-indole-6-carbonyl azide

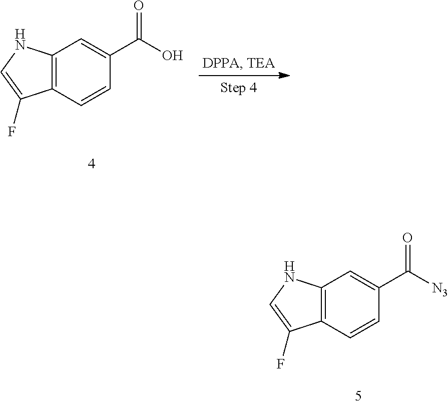

To a solution of 3-fluoro-1H-indole-6-carboxylic acid (150.0 mg, 0.8 mmol, 1.0 equiv) in THF (2.0 mL), DPPA (345.6 mg, 1.3 mmol, 1.5 equiv) and TEA (169.5 mg, 1.7 mmol, 2.0 equiv) were added. The resulting solution was stirred for 3 hr at room temperature, then quenched by the addition of water. The resulting solution was extracted with ethyl acetate and concentrated under vacuum to give 3-fluoro-1H-indole-6-carbonyl azide (150 mg, 87.7%) as a yellow solid, which was used to next step without further purification.

### 5. Synthesis of 3-(3-fluoro-1H-indol-6-yl)-1-[4-(trifluoromethyl)phenyl]urea

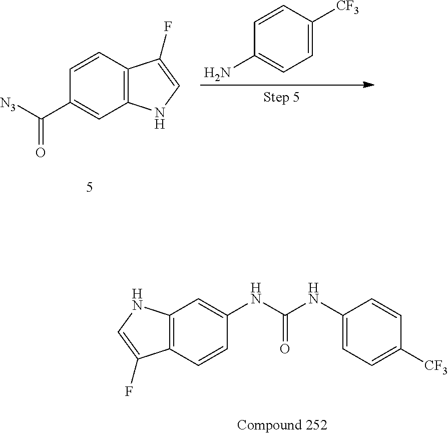 [SMILES: [N-]=[N+]=NC(=O)c1ccc2c(F)c[nH]c2c1 ; Nc1ccc(C(F)(F)F)cc1 ; O=C(Nc1ccc(C(F)(F)F)cc1)Nc1ccc2c(F)c[nH]c2c1]

To a solution of 3-fluoro-1H-indole-6-carbonyl azide (150.0 mg, 0.7 mmol, 1.0 equiv) in toluene (5.0 mL), 3-fluoro-1H-indol-6-amine (132.4 mg, 0.9 mmol, 1.2 equiv) and TEA (223.0 mg, 2.2 mmol, 3.0 equiv) were added. The resulting solution was stirred for 2 hrs at 100° C. and then concentrated under vacuum. The crude product was purified by Prep-HPLC with the following conditions: Column, YMC-Actus Triart C18, 30*250.5 um; mobile phase, Water (10 MMOL/L NH₄HCO₃+0.1% NH₃.H₂O) and ACN (50% Phase B up to 70% in 7 min); Detector, UV254 nm. This resulted in 3-(3-fluoro-1H-indol-6-yl)-1-[4-(trifluoromethyl)phenyl]urea (81.6 mg, 32.9%) as a white solid. LCMS Method AC: [M+H]⁺=338. ¹H NMR (300 MHz, DMSO-d₆) δ 10.69 (s, 1H), 9.06 (s, 1H), 8.79 (s, 1H), 7.80 (s, 1H), 7.70-7.63 (m, 4H), 7.43 (d, J=8.4 Hz, 1H), 7.21 (t, J=2.7 Hz, 1H), 6.95 (d, J=8.4 Hz, 1H).

### Example 3: Synthesis of N-(1H-indol-6-yl)-1,6-naphthyridin-5-amine (Compound 478)

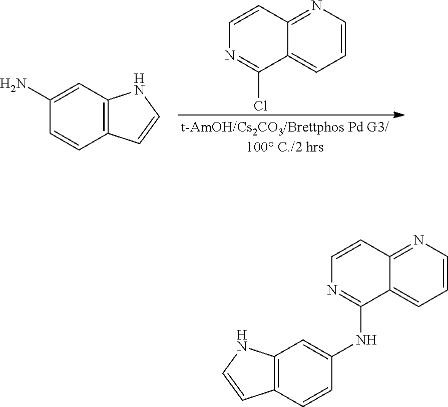 [SMILES: Nc1ccc2cc[nH]c2c1 ; Clc1nccc2ncccc12 ; c1cnc2ccnc(Nc3ccc4cc[nH]c4c3)c2c1]

### Procedure:

1H-indol-6-amine (52.8 mg, 0.4 mmol, 1.0 equiv.) and 5-chloro-1,6-naphthyridine(65.6 mg, 0.4 mmol, 1.00 equiv.) were dissolved in t-AmOH (4.0 mL). Cs₂CO₃ (390 mg, 1.20 mmol, 3.0 equiv.) and Brettphos Pd G3 (0.05 equiv.) were then added under N2 atmosphere. The mixture was stirred at 100° C. for 2 hours. 2.0 mL water was added to the reaction mixture and was extracted with EtOAc. The organic layer was collected and concentrated solvent by Speedvac. The residue was purified by prep HPLC to give N-(1H-indol-6-yl)-1,6-naphthyridin-5-amine (37.2 mg, 0.14 mmol) as solid. MS-ESI, 261.1 [M+H⁺].

¹H NMR (400 MHz, DMSO-d₆) δ ppm 11.02 (br s, 1H) 9.37 (br s, 1H) 8.96-9.04 (m, 2H) 8.10-8.18 (m, 2H) 7.57-7.65 (m, 1H) 7.49 (d, 1H) 7.25-7.35 (m, 2H) 7.17 (d, 1H) 6.38 (t, 1H)

TABLE E1
The compounds in Table E1 were prepared using the above procedure.

| Example | Compound | Final | | LC-MS, MS-ESI, |
|---|---|---|---|---|
| # | # | compound | IUPAC Name | --[M + H⁺]. |
| 4 | 391 | 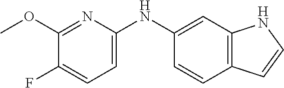 [SMILES: COc1nc(Nc2ccc3cc[nH]c3c2)ccc1F] | N-(5-fluoro-6- methoxypyridin-2-yl)- 1H-indol-6-amine | 258 |
| 5 | 392 | 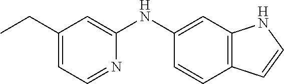 [SMILES: CCc1ccnc(Nc2ccc3cc[nH]c3c2)c1] | N-(4-ethylpyridin-2-yl)- 1H-indol-6-amine | 238.1 |
| 6 | 393 | 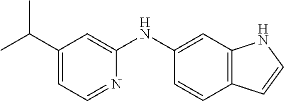 [SMILES: CC(C)c1ccnc(Nc2ccc3cc[nH]c3c2)c1] | N-(4-isopropylpyridin-2- yl)-1H-indol-6-amine | 252.2 |
| 7 | 394 | 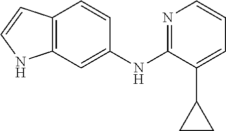 [SMILES: c1cnc(Nc2ccc3cc[nH]c3c2)c(C2CC2)c1] | N-(3-cyclopropylpyridin- 2-yl)-1H-indol-6-amine | 250.2 |
| 8 | 395 | 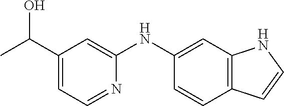 [SMILES: CC(O)c1ccnc(Nc2ccc3cc[nH]c3c2)c1] | 1-(2-((1H-indol-6- yl)amino)pyridin-4- yl)ethanol | 254.2 |
| 9 | 396 | 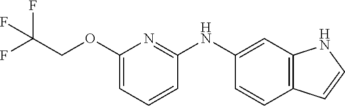 [SMILES: FC(F)(F)COc1cccc(Nc2ccc3cc[nH]c3c2)n1] | N-(6-(2,2,2- trifluoroethoxy)pyridin- 2-yl)-1H-indol-6-amine | 308.1 |
| 10 | 397 | 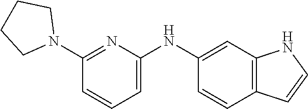 [SMILES: c1cc(Nc2ccc3cc[nH]c3c2)nc(N2CCCC2)c1] | N-(6-(pyrrolidin-1- yl)pyridin-2-yl)-1H- indol-6-amine | 279.1 |
| 11 | 398 | 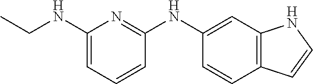 [SMILES: CCNc1cccc(Nc2ccc3cc[nH]c3c2)n1] | N2-ethyl-N6-(1H-indol- 6-yl)pyridine-2,6- diamine | 253.1 |
| 12 | 399 | 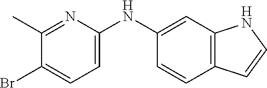 [SMILES: Cc1nc(Nc2ccc3cc[nH]c3c2)ccc1Br] | N-(5-bromo-6- methylpyridin-2-yl)-1H- indol-6-amine | 302.1 |
| 13 | 400 | 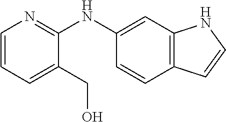 [SMILES: OCc1cccnc1Nc1ccc2cc[nH]c2c1] | (2-((1H-indol-6- yl)amino)pyridin-3- yl)methanol | 240.2 |
| 14 | 401 | 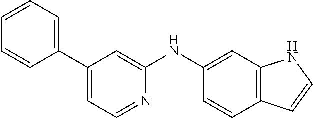 [SMILES: c1ccc(-c2ccnc(Nc3ccc4cc[nH]c4c3)c2)cc1] | N-(4-phenylpyridin-2- yl)-1H-indol-6-amine | 286.2 |
| 15 | 402 | 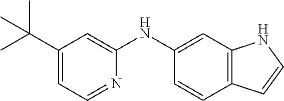 [SMILES: CC(C)(C)c1ccnc(Nc2ccc3cc[nH]c3c2)c1] | N-(4-(tert-butyl)pyridin- 2-yl)-1H-indol-6-amine | 266.2 |
| 16 | 403 | 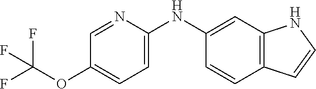 [SMILES: FC(F)(F)Oc1ccc(Nc2ccc3cc[nH]c3c2)nc1] | N-(5- (trifluoromethoxy) pyridin-2-yl)-1H-indol- 6-amine | 294.1 |
| 17 | 404 | 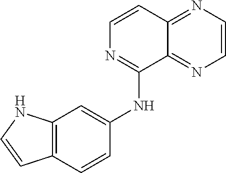 [SMILES: c1cc2nccnc2c(Nc2ccc3cc[nH]c3c2)n1] | N-(1H-indol-6- yl)pyrido[3,4-b]pyrazin- 5-amine | 262.2 |
| 18 | 405 | 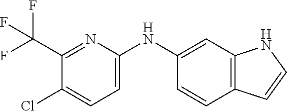 [SMILES: FC(F)(F)c1nc(Nc2ccc3cc[nH]c3c2)ccc1Cl] | N-(5-chloro-6- (trifluoromethyl)pyridin- 2-yl)-1H-indol-6-amine | 312.1 |
| 19 | 406 | 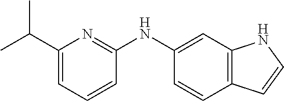 [SMILES: CC(C)c1cccc(Nc2ccc3cc[nH]c3c2)n1] | N-(6-isopropylpyridin-2- yl)-1H-indol-6-amine | 252.2 |
| 20 | 407 | 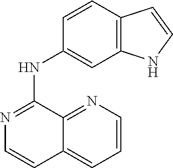 [SMILES: c1cnc2c(Nc3ccc4cc[nH]c4c3)nccc2c1] | N-(1H-indol-6-yl)-1,7- naphthyridin-8-amine | 261.2 |
| 21 | 408 | 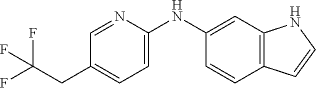 [SMILES: FC(F)(F)Cc1ccc(Nc2ccc3cc[nH]c3c2)nc1] | N-(5-(2,2,2- trifluoroethyl)pyridin-2- yl)-1H-indol-6-amine | 292.1 |
| 22 | 409 | 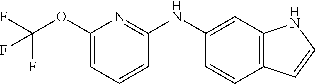 [SMILES: FC(F)(F)Oc1cccc(Nc2ccc3cc[nH]c3c2)n1] | N-(6- (trifluoromethoxy) pyridin-2-yl)-1H-indol- 6-amine | 294.1 |
| 23 | 410 | 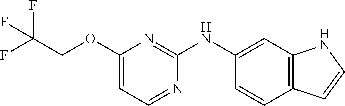 [SMILES: FC(F)(F)COc1ccnc(Nc2ccc3cc[nH]c3c2)n1] | N-(4-(2,2,2- trifluoroethoxy) pyrimidin- 2-yl)-1H-indol- 6-amine | 309.1 |
| 24 | 411 | 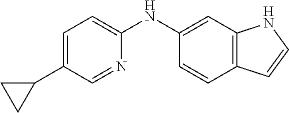 [SMILES: c1cc2ccc(Nc3ccc(C4CC4)cn3)cc2[nH]1] | N-(5-cyclopropylpyridin- 2-yl)-1H-indol-6-amine | 250.2 |
| 25 | 412 | 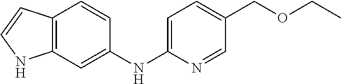 [SMILES: CCOCc1ccc(Nc2ccc3cc[nH]c3c2)nc1] | N-(5- (ethoxymethyl)pyridin-2- yl)-1H-indol-6-amine | 268.2 |
| 26 | 413 | 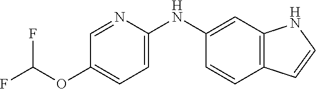 [SMILES: FC(F)Oc1ccc(Nc2ccc3cc[nH]c3c2)nc1] | N-(5- (difluoromethoxy) pyridin-2-yl)-1H-indol- 6-amine | 276.1 |
| 27 | 414 | 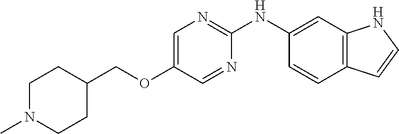 [SMILES: CN1CCC(COc2cnc(Nc3ccc4cc[nH]c4c3)nc2)CC1] | N-(5-((1- methylpiperidin-4- yl)methoxy)pyrimidin-2- yl)-1H-indol-6-amine | 338.3 |
| 28 | 415 | 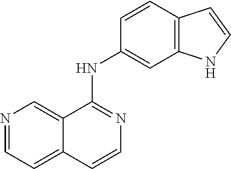 [SMILES: c1cc2ccnc(Nc3ccc4cc[nH]c4c3)c2cn1] | N-(1H-indol-6-yl)-2,7- naphthyridin-1-amine | 261.2 |
| 29 | 416 | 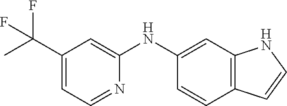 [SMILES: CC(F)(F)c1ccnc(Nc2ccc3cc[nH]c3c2)c1] | N-(4-(1,1- difluoroethyl)pyridin-2- yl)-1H-indol-6-amine | 274.2 |
| 30 | 417 | 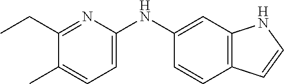 [SMILES: CCc1nc(Nc2ccc3cc[nH]c3c2)ccc1C] | N-(6-ethyl-5- methylpyridin-2-yl)-1H- indol-6-amine | |
| 31 | 418 | 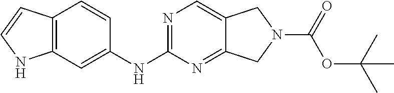 [SMILES: CC(C)(C)OC(=O)N1Cc2cnc(Nc3ccc4cc[nH]c4c3)nc2C1] | tert-butyl 2-((1H-indol-6- yl)amino)-5H- pyrrolo[3,4- d]pyrimidine-6(7H)- carboxylate | 352.2 |
| 32 | 419 | 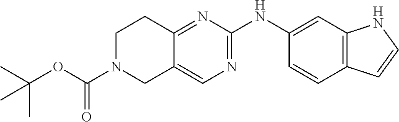 [SMILES: CC(C)(C)OC(=O)N1CCc2nc(Nc3ccc4cc[nH]c4c3)ncc2C1] | tert-butyl 2-((1H-indol-6- yl)amino)-7,8- dihydropyrido[4,3- d]pyrimidine-6(5H)- carboxylate | 366.3 |
| 33 | 420 | 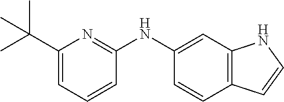 [SMILES: CC(C)(C)c1cccc(Nc2ccc3cc[nH]c3c2)n1] | N-(6-(tert-butyl)pyridin- 2-yl)-1H-indol-6-amine | 266.2 |
| 34 | 421 | 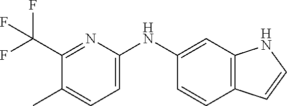 [SMILES: Cc1ccc(Nc2ccc3cc[nH]c3c2)nc1C(F)(F)F] | N-(5-methyl-6- (trifluoromethyl)pyridin- 2-yl)-1H-indol-6-amine | 292.2 |
| 35 | 422 | 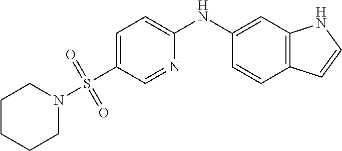 [SMILES: O=S(=O)(c1ccc(Nc2ccc3cc[nH]c3c2)nc1)N1CCCCC1] | N-(5-(piperidin-1- ylsulfonyl)pyridin-2-yl)- 1H-indol-6-amine | 357.2 |
| 36 | 423 | 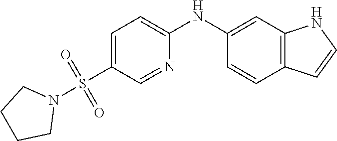 [SMILES: O=S(=O)(c1ccc(Nc2ccc3cc[nH]c3c2)nc1)N1CCCC1] | N-(5-(pyrrolidin-1- ylsulfonyl)pyridin-2-yl)- 1H-indol-6-amine | 343.2 |
| 37 | 424 | 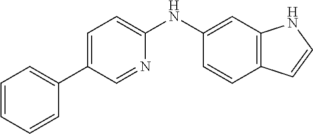 [SMILES: c1ccc(-c2ccc(Nc3ccc4cc[nH]c4c3)nc2)cc1] | N-(5-phenylpyridin-2- yl)-1H-indol-6-amine | 286.2 |
| 38 | 425 | 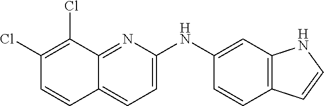 [SMILES: Clc1ccc2ccc(Nc3ccc4cc[nH]c4c3)nc2c1Cl] | 7,8-dichloro-N-(1H- indol-6-yl)quinolin-2- amine | 328 |
| 39 | 426 | 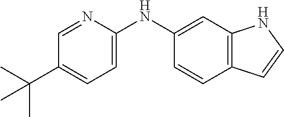 [SMILES: CC(C)(C)c1ccc(Nc2ccc3cc[nH]c3c2)nc1] | N-(5-(tert-butyl)pyridin- 2-yl)-1H-indol-6-amine | 266.2 |
| 40 | 427 | 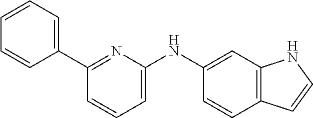 [SMILES: c1ccc(-c2cccc(Nc3ccc4cc[nH]c4c3)n2)cc1] | N-(6-phenylpyridin-2- yl)-1H-indol-6-amine | 286.2 |
| 41 | 428 | 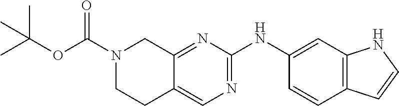 [SMILES: CC(C)(C)OC(=O)N1CCc2cnc(Nc3ccc4cc[nH]c4c3)nc2C1] | tert-butyl 2-((1H-indol-6- yl)amino)-5,6- dihydropyrido[3,4- d]pyrimidine-7(8H)- carboxylate | 366.2 |
| 42 | 429 | 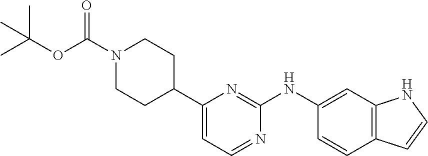 [SMILES: CC(C)(C)OC(=O)N1CCC(c2ccnc(Nc3ccc4cc[nH]c4c3)n2)CC1] | tert-butyl4-(2-((1H- indol-6- yl)amino)pyrimidin-4- yl)piperidine-1- carboxylate | 394.3 |
| 43 | 430 | 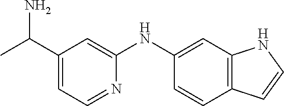 [SMILES: CC(N)c1ccnc(Nc2ccc3cc[nH]c3c2)c1] | N-(4-(1- aminoethyl)pyridin-2- yl)-1H-indol-6-amine | 253.2 |
| 44 | 431 | 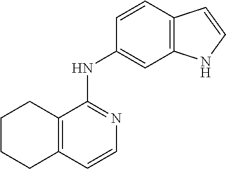 [SMILES: c1cc2c(c(Nc3ccc4cc[nH]c4c3)n1)CCCC2] | N-(1H-indol-6-yl)- 5,6,7,8- tetrahydroisoquinolin-1- amine | 264.2 |
| 45 | 432 | 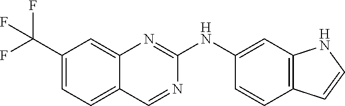 [SMILES: FC(F)(F)c1ccc2cnc(Nc3ccc4cc[nH]c4c3)nc2c1] | N-(1H-indol-6-yl)-7- (trifluoromethyl)quinazol in-2-amine | 329.2 |
| 46 | 433 | 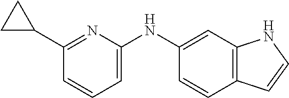 [SMILES: c1cc(Nc2ccc3cc[nH]c3c2)nc(C2CC2)c1] | N-(6-cyclopropylpyridin- 2-yl)-1H-indol-6-amine | 250.2 |

### Example 47: Synthesis of N-(4-(2,2,2-trifluoroethoxy)pyrimidin-2-yl)-1H-indol-5-amine (Compound 434)

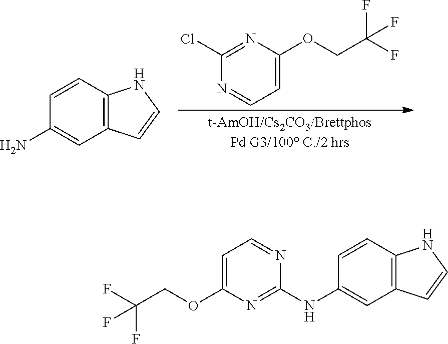 [SMILES: Nc1ccc2[nH]ccc2c1 ; FC(F)(F)COc1ccnc(Cl)n1 ; FC(F)(F)COc1ccnc(Nc2ccc3[nH]ccc3c2)n1]

### Procedure:

1H-indol-5-amine (52.8 mg, 0.4 mmol, 1.0 equiv.) and 2-chloro-4-(2,2,2-trifluoroethoxy) pyrimidine (84.8 mg, 0.4 mmol, 1.0 equiv.) were dissolved in t-AmOH (4.0 mL), then Cs₂CO₃ (390 mg, 1.2 mmol, 3.0 equiv.) and Brettphos Pd G3 (0.05 equiv.) were added under N2 atmosphere. The mixture was stirred at 100° C. for 2 hours. 2.0 mL water was added to the reaction mixture and was extracted with EtOAc. The organic layer was collected and concentrated solvent by Speedvac. The residue was purified by prep HPLC to give N-(4-(2,2,2-trifluoroethoxy)pyrimidin-2-yl)-1H-indol-5-amine (63.9 mg, 0.21 mmol) as solid. MS-ESP, 309.1 [M+HG].

¹H NMR (400 MHz, DMSO-d₆) δ ppm 10.95 (br s, 1H) 9.43 (s, 1H) 8.25 (d, 1H) 7.88 (s, 1H) 7.25-7.38 (m, 3H) 6.29-6.40 (m, 2H) 5.04 (q, 2H)

TABLE E2
The compounds in Table E2 were prepared using the above procedure.

| Example | | | IUPAC | LC-MS, MS-ESI, |
|---|---|---|---|---|
| # | | Structure | Name | --[M + H⁺]. |
| 48 | 435 | 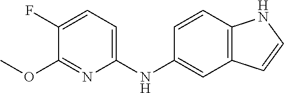 [SMILES: COc1nc(Nc2ccc3[nH]ccc3c2)ccc1F] | N-(5-fluoro-6- methoxypyridin-2- yl)-1H-indol-5- amine | 258.1 |
| 49 | 436 | 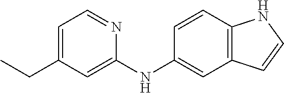 [SMILES: CCc1ccnc(Nc2ccc3[nH]ccc3c2)c1] | N-(4-ethylpyridin- 2-yl)-1H-indol-5- amine | 238.2 |
| 50 | 437 | 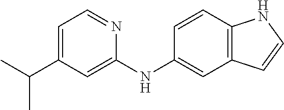 [SMILES: CC(C)c1ccnc(Nc2ccc3[nH]ccc3c2)c1] | N-(4- isopropylpyridin-2- yl)-1H-indol-5- amine | 252.2 |
| 51 | 438 | 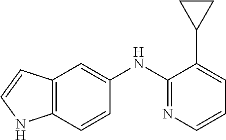 [SMILES: c1cnc(Nc2ccc3[nH]ccc3c2)c(C2CC2)c1] | N-(3- cyclopropylpyridin- 2-yl)-1H-indol-5- amine | 250.2 |
| 52 | 439 | 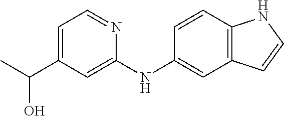 [SMILES: CC(O)c1ccnc(Nc2ccc3[nH]ccc3c2)c1] | 1-(2-((1H-indol-5- yl)amino)pyridin-4- yl)ethanol | 254.2 |
| 53 | 440 | 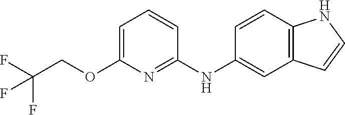 [SMILES: FC(F)(F)COc1cccc(Nc2ccc3[nH]ccc3c2)n1] | N-(6-(2,2,2- trifluoroethoxy) pyridin-2-yl)-1H- indol-5-amine | 308.1 |
| 54 | 441 | 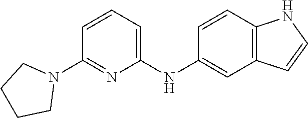 [SMILES: c1cc(Nc2ccc3[nH]ccc3c2)nc(N2CCCC2)c1] | N-(6-(pyrrolidin-1- yl)pyridin-2-yl)- 1H-indol-5-amine | 279.2 |
| 55 | 442 | 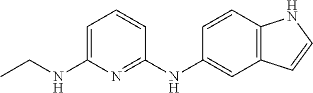 [SMILES: CCNc1cccc(Nc2ccc3[nH]ccc3c2)n1] | N2-ethyl-N6-(1H- indol-5-yl)pyridine- 2,6-diamine | 253.2 |
| 56 | 443 | 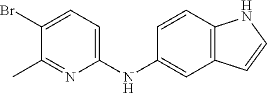 [SMILES: Cc1nc(Nc2ccc3[nH]ccc3c2)ccc1Br] | N-(5-bromo-6- methylpyridin-2- yl)-1H-indol-5- amine | 302.1 |
| 57 | 444 | 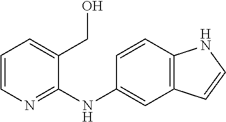 [SMILES: OCc1cccnc1Nc1ccc2[nH]ccc2c1] | (2-((1H-indol-5- yl)amino)pyridin-3- yl)methanol | 240.2 |
| 58 | 445 | 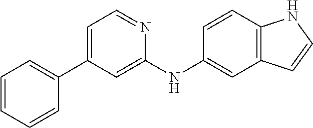 [SMILES: c1ccc(-c2ccnc(Nc3ccc4[nH]ccc4c3)c2)cc1] | N-(4- phenylpyridin-2- yl)-1H-indol-5- amine | 286.2 |
| 59 | 446 | 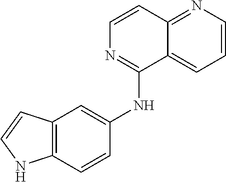 [SMILES: c1cnc2ccnc(Nc3ccc4[nH]ccc4c3)c2c1] | N-(1H-indol-5-yl)- 1,6-naphthyridin-5- amine | 261.1 |
| 60 | 447 | 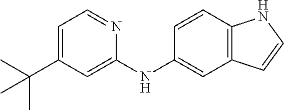 [SMILES: CC(C)(C)c1ccnc(Nc2ccc3[nH]ccc3c2)c1] | N-(4-(tert- butyl)pyridin-2-yl)- 1H-indol-5-amine | 266.2 |
| 61 | 448 | 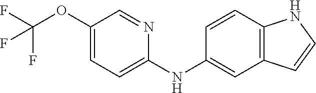 [SMILES: FC(F)(F)Oc1ccc(Nc2ccc3[nH]ccc3c2)nc1] | N-(5- (trifluoromethoxy) pyridin-2-yl)-1H- indol-5-amine | 294.1 |
| 62 | 449 | 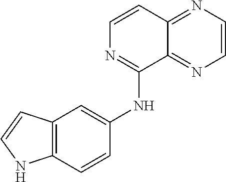 [SMILES: c1cc2nccnc2c(Nc2ccc3[nH]ccc3c2)n1] | N-(1H-indol-5- yl)pyrido[3,4- b]pyrazin-5-amine | 262.2 |
| 63 | 450 | 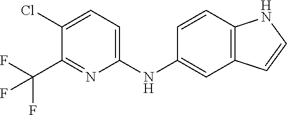 [SMILES: FC(F)(F)c1nc(Nc2ccc3[nH]ccc3c2)ccc1Cl] | N-(5-chloro-6- (trifluoromethyl) pyridin-2-yl)-1H- indol-5-amine | 312.1 |
| 64 | 451 | 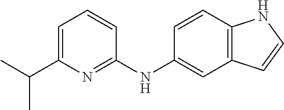 [SMILES: CC(C)c1cccc(Nc2ccc3[nH]ccc3c2)n1] | N-(6- isopropylpyridin-2- yl)-1H-indol-5- amine | 252.2 |
| 65 | 452 | 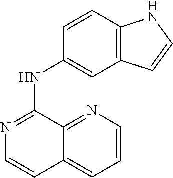 [SMILES: c1cnc2c(Nc3ccc4[nH]ccc4c3)nccc2c1] | N-(1H-indol-5-yl)- 1,7-naphthyridin-8- amine | 261.1 |
| 66 | 453 | 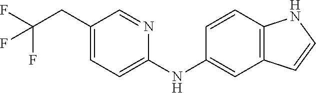 [SMILES: FC(F)(F)Cc1ccc(Nc2ccc3[nH]ccc3c2)nc1] | N-(5-(2,2,2- trifluoroethyl)pyridin- 2-yl)-1H-indol-5- amine | 292.2 |
| 67 | 454 | 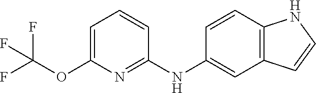 [SMILES: FC(F)(F)Oc1cccc(Nc2ccc3[nH]ccc3c2)n1] | N-(6- (trifluoromethoxy) pyridin-2-yl)-1H- indol-5-amine | 294 |
| 68 | 455 | 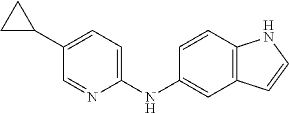 [SMILES: c1cc2cc(Nc3ccc(C4CC4)cn3)ccc2[nH]1] | N-(5- cyclopropylpyridin- 2-yl)-1H-indol-5- amine | 250.2 |
| 69 | 456 | 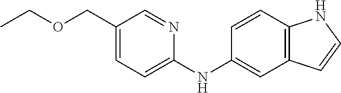 [SMILES: CCOCc1ccc(Nc2ccc3[nH]ccc3c2)nc1] | N-(5- (ethoxymethyl) pyridin-2-yl)-1H- indol-5-amine | 268.2 |
| 70 | 457 | 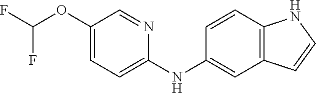 [SMILES: FC(F)Oc1ccc(Nc2ccc3[nH]ccc3c2)nc1] | N-(5- (difluoromethoxy) pyridin-2-yl)-1H- indol-5-amine | 276.2 |
| 71 | 458 | 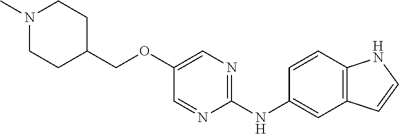 [SMILES: CN1CCC(COc2cnc(Nc3ccc4[nH]ccc4c3)nc2)CC1] | N-(5-((1- methylpiperidin-4- yl)methoxy)pyrimid in-2-yl)-1H-indol- 5-amine | 338.1 |
| 72 | 459 | 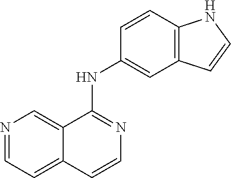 [SMILES: c1cc2ccnc(Nc3ccc4[nH]ccc4c3)c2cn1] | N-(1H-indol-5-yl)- 2,7-naphthyridin-1- amine | 261.2 |
| 73 | 460 | 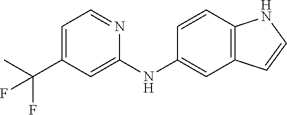 [SMILES: CC(F)(F)c1ccnc(Nc2ccc3[nH]ccc3c2)c1] | N-(4-(1,1- difluoroethyl) pyridin-2-yl)-1H- indol-5-amine | 274.2 |
| 74 | 461 | 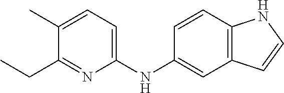 [SMILES: CCc1nc(Nc2ccc3[nH]ccc3c2)ccc1C] | N-(6-ethyl-5- methylpyridin-2- yl)-1H-indol-5- amine | |
| 75 | 462 | 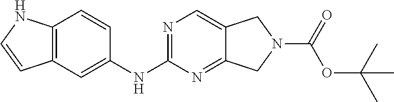 [SMILES: CC(C)(C)OC(=O)N1Cc2cnc(Nc3ccc4[nH]ccc4c3)nc2C1] | tert-butyl 2-((1H- indol-5-yl)amino)- 5H-pyrrolo[3,4- d]pyrimidine- 6(7H)-carboxylate | 352.2 |
| 76 | 463 | 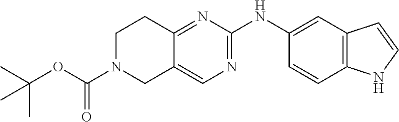 [SMILES: CC(C)(C)OC(=O)N1CCc2nc(Nc3ccc4[nH]ccc4c3)ncc2C1] | tert-butyl 2-((1H- indol-5-yl)amino)- 7,8- dihydropyrido[4,3- d]pyrimidine- 6(5H)-carboxylate | 366.3 |
| 77 | 464 | 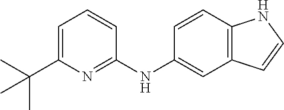 [SMILES: CC(C)(C)c1cccc(Nc2ccc3[nH]ccc3c2)n1] | N-(6-(tert- butyl)pyridin-2-yl)- 1H-indol-5-amine | 266.2 |
| 78 | 465 | 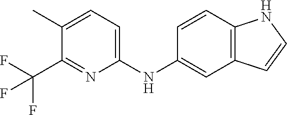 [SMILES: Cc1ccc(Nc2ccc3[nH]ccc3c2)nc1C(F)(F)F] | N-(5-methyl-6- (trifluoromethyl) pyridin-2-yl)-1H- indol-5-amine | 292.2 |
| 79 | 466 | 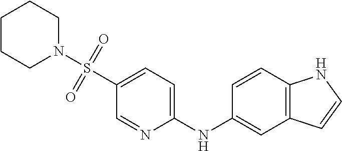 [SMILES: O=S(=O)(c1ccc(Nc2ccc3[nH]ccc3c2)nc1)N1CCCCC1] | N-(5-(piperidin-1- ylsulfonyl)pyridin- 2-yl)-1H-indol-5- amine | 357.2 |
| 80 | 467 | 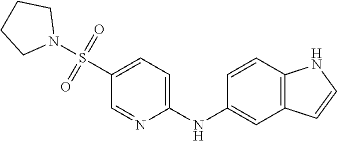 [SMILES: O=S(=O)(c1ccc(Nc2ccc3[nH]ccc3c2)nc1)N1CCCC1] | N-(5-(pyrrolidin-1- ylsulfonyl)pyridin- 2-yl)-1H-indol-5- amine | 343.2 |
| 81 | 468 | 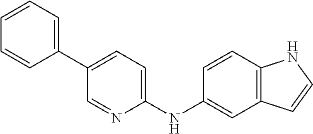 [SMILES: c1ccc(-c2ccc(Nc3ccc4[nH]ccc4c3)nc2)cc1] | N-(5- phenylpyridin-2- yl)-1H-indol-5- amine | 286.1 |
| 82 | 469 | 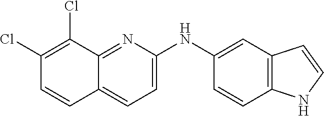 [SMILES: Clc1ccc2ccc(Nc3ccc4[nH]ccc4c3)nc2c1Cl] | 7,8-dichloro-N- (1H-indol-5- yl)quinolin-2-amine | 328.1 |
| 83 | 470 | 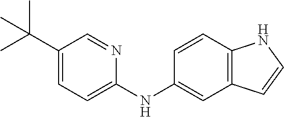 [SMILES: CC(C)(C)c1ccc(Nc2ccc3[nH]ccc3c2)nc1] | N-(5-(tert- butyl)pyridin-2-yl)- 1H-indol-5-amine | 266.2 |
| 84 | 471 | 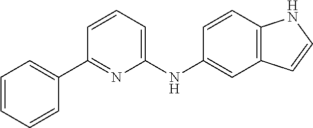 [SMILES: c1ccc(-c2cccc(Nc3ccc4[nH]ccc4c3)n2)cc1] | N-(6- phenylpyridin-2- yl)-1H-indol-5- amine | 286.2 |
| 85 | 472 | 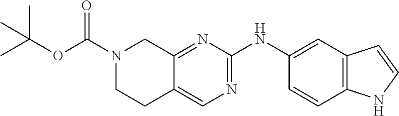 [SMILES: CC(C)(C)OC(=O)N1CCc2cnc(Nc3ccc4[nH]ccc4c3)nc2C1] | tert-butyl 2-((1H- indol-5-yl)amino)- 5,6- dihydropyrido[3,4- d]pyrimidine- 7(8H)-carboxylate | 366.2 |
| 86 | 473 | 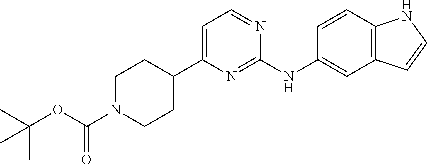 [SMILES: CC(C)(C)OC(=O)N1CCC(c2ccnc(Nc3ccc4[nH]ccc4c3)n2)CC1] | tert-butyl4-(2- ((1H-indol-5- yl)amino)pyrimidin- 4-yl)piperidine-1- carboxylate | 394.3 |
| 87 | 474 | 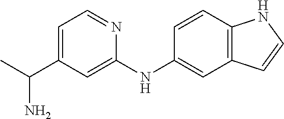 [SMILES: CC(N)c1ccnc(Nc2ccc3[nH]ccc3c2)c1] | N-(4-(1- aminoethyl)pyridin- 2-yl)-1H-indol-5- amine | 253.2 |
| 88 | 475 | 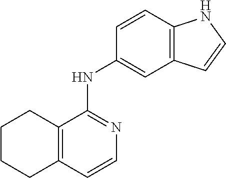 [SMILES: c1cc2c(c(Nc3ccc4[nH]ccc4c3)n1)CCCC2] | N-(1H-indol-5-yl)- 5,6,7,8- tetrahydroisoquinolin- 1-amine | 264.2 |
| 89 | 476 | 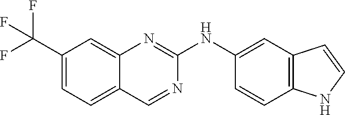 [SMILES: FC(F)(F)c1ccc2cnc(Nc3ccc4[nH]ccc4c3)nc2c1] | N-(1H-indol-5-yl)- 7-(trifluoromethyl) quinazolin-2-amine | 329.2 |
| 90 | 477 | 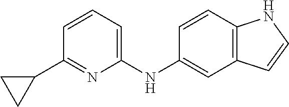 [SMILES: c1cc(Nc2ccc3[nH]ccc3c2)nc(C2CC2)c1] | N-(6- cyclopropylpyridin- 2-yl)-1H-indol-5- amine | 250.2 |

### Example 90: Synthesis of 1-(2-ethylphenyl)-3-(1H-indol-5-yl)urea (Compound 363)

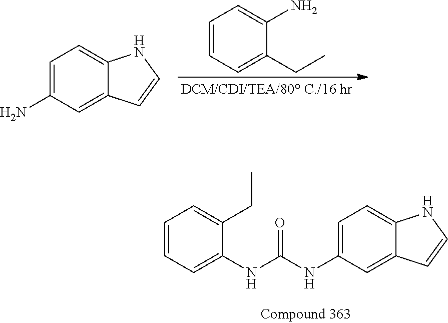

CDI (48.6 mg, 0.3 mmol, 1.0 equiv.) was dissolved in DCM (2.0 mL), then the solution of 1H-indol-5-amine (39.6 mg, 0.3 mmol, 1.0 equiv.) and TEA (87.0 μL, 0.6 mmol, 2.0 equiv.) in DCM (2.0 mL) were added in one portion at −30° C. The mixture was stirred at −30° C. for 30 mins, then heated to 20° C. and stirred for 10 mins. Then the mixture was added 2-ethylaniline (54.5 mg, 0.45 mmol, 1.5 equiv.) and TEA (87.0 uL, 0.6 mmol, 2.0 equiv.) in DCM (1.0 mL) in one portion at 20° C., and the mixture was stirred at 80° C. for 16 hours. The reaction mixture was concentrated by speedvac. The residue was purified by prep HPLC to give 1-(2-ethylphenyl)-3-(1H-indol-5-yl)urea (17.7 mg, 0.063 mmol). MS-ESI, 280.2 [M+H⁺].

¹H NMR (400 MHz, DMSO-d₆) δ ppm 10.93 (br s, 1H) 8.76 (s, 1H) 7.81-7.88 (m, 1H) 7.78 (s, 1H) 7.70 (d, 1H) 7.26-7.33 (m, 2H) 7.09-7.20 (m, 2H) 7.07 (dd, 1H) 6.97 (td, 1H) 6.35 (br s, 1H) 2.62 (q, 2H) 1.18 (t, 3H)

The following compounds were synthesized using methods similar to that described above *LC/MS Method: Shim-pack XR-ODS, C18, 3×50 mm, 2.5 um column, 1.0 uL injection, 1.5 mL/min flow rate, 90-900 amu scan range, 190-400 nm UV range, 5-100% (1.1 min), 100% (0.6 min) gradient with ACN (0.05% TFA) and water (0.05% TFA), with 2.0 minute as total run time.

| Compound Number | LC/MS* |
|---|---|
| 153 | 372.1 |
| 154 | 384 |
| 155 | 399.9 |
| 156 | 400 |
| 157 | 350.2 |
| 158 | 378 |
| 159 | 359.2 |
| 160 | 380.2 |
| 161 | 292.2 |
| 162 | 244.2 |
| 163 | 281.2 |
| 164 | 288.1 |
| 165 | 376.1 |
| 166 | 364.1 |
| 167 | 386.1 |
| 168 | 385.2 |
| 169 | 396.1 |
| 170 | 291.2 |
| 171 | 322.2 |
| 172 | 362.2 |
| 173 | 403.2 |
| 174 | 377.3 |
| 175 | 374.1 |
| 176 | 365.2 |
| 177 | 363.2 |
| 178 | 359.2 |
| 179 | 359.2 |
| 180 | 284.2 |
| 181 | 360.1 |
| 182 | 378.2 |
| 183 | 364.1 |
| 184 | 358.1 |
| 185 | 284.2 |
| 186 | 308.2 |
| 187 | 280.2 |
| 188 | 246.2 |
| 189 | 256.1 |
| 190 | 243.2 |
| 191 | 243.1 |
| 192 | 280.2 |
| 193 | 295.2 |
| 194 | 286.1 |
| 195 | 375.2 |
| 196 | 398 |
| 197 | 400 |
| 198 | 359.2 |
| 199 | 362.2 |
| 200 | 384.1 |
| 201 | 383.1 |
| 202 | 359.2 |
| 203 | 384.1 |
| 204 | 394.1 |
| 205 | 368.1 |
| 206 | 368.1 |
| 207 | 368.1 |
| 208 | 358.1 |
| 209 | 332.2 |
| 210 | 314.1 |
| 211 | 314.2 |
| 212 | 294.2 |
| 213 | 294.2 |
| 214 | 292.2 |
| 215 | 246.2 |
| 216 | 244.2 |
| 217 | 299.2 |
| 218 | 298.2 |
| 219 | 268.1 |
| 220 | 254.1 |
| 221 | 302.2 |
| 222 | 294.2 |
| 223 | 294.2 |
| 224 | 280.2 |
| 225 | 280.2 |
| 226 | 304.1 |
| 227 | 291.2 |
| 228 | 318.1 |
| 229 | 280.2 |
| 230 | 270.1 |
| 231 | 266.2 |
| 233 | 378.1 |
| 234 | 294.2 |
| 235 | 400.4 |
| 236 | 372.4 |
| 237 | 364.3 |
| 240 | 360.1 |
| 243 | 328.3 |
| 244 | 354 |
| 245 | 345 |
| 246 | 338.15 |
| 247 | 372.1 |
| 248 | 359.1 |
| 249 | 372.25 |
| 250 | 321.2 |
| 251 | 354.1 |
| 252 | 338.1 |
| 253 | 338.1 |
| 254 | 354.1 |
| 255 | 343 |
| 256 | 396 |
| 257 | 362.1 |
| 259 | 334.1 |
| 260 | 348.3 |
| 261 | 442.1 |
| 262 | 338 |
| 263 | 374.2 |
| 264 | 334.1 |
| 265 | 334.1 |
| 266 | 328.2 |
| 267 | 334 |
| 268 | 334 |
| 269 | 321 |
| 270 | 308.1 |
| 271 | 314.2 |
| 272 | 320.1 |
| 273 | 308.2 |
| 274 | 384.1 |
| 275 | 392 |
| 276 | 359.2 |
| 277 | 429.2 |
| 278 | 380.2 |
| 279 | 378.2 |
| 280 | 254.2 |
| 281 | 243.1 |
| 282 | 385.2 |
| 283 | 291.2 |
| 284 | 385.2 |
| 285 | 371.1 |
| 286 | 371.1 |
| 287 | 371.1 |
| 288 | 362.2 |
| 289 | 384.1 |
| 290 | 383.1 |
| 291 | 378.1 |
| 292 | 359.2 |
| 293 | 407.2 |
| 294 | 377.3 |
| 295 | 374.1 |
| 296 | 365.2 |
| 297 | 363.2 |
| 298 | 363.2 |
| 299 | 360.1 |
| 300 | 359.1 |
| 301 | 284.2 |
| 302 | 297.2 |
| 303 | 302.2 |
| 304 | 358.1 |
| 305 | 299.2 |
| 306 | 256.1 |
| 307 | 295.2 |
| 308 | 375.2 |
| 309 | 360.1 |
| 311 | 322.1 |
| 312 | 359.2 |
| 313 | 362.2 |
| 314 | 403.2 |
| 315 | 384.1 |
| 316 | 359.2 |
| 317 | 360.1 |
| 318 | 368.1 |
| 319 | 368.1 |
| 320 | 368.1 |
| 321 | 364.2 |
| 322 | 284.2 |
| 323 | 332.1 |
| 324 | 294.2 |
| 325 | 294.2 |
| 326 | 292.2 |
| 327 | 280.2 |
| 328 | 246.2 |
| 329 | 246.2 |
| 330 | 244.2 |
| 331 | 244.2 |
| 332 | 298.2 |
| 333 | 268.2 |
| 334 | 243.1 |
| 335 | 281.2 |
| 336 | 302.1 |
| 337 | 294.2 |
| 338 | 280.2 |
| 339 | 280.2 |
| 340 | 280.1 |
| 341 | 304.1 |
| 342 | 288.2 |
| 343 | 318.1 |
| 345 | 386.1 |
| 346 | 378.1 |
| 347 | 294.2 |
| 348 | 400.4 |
| 349 | 394.1 |
| 350 | 383.1 |
| 351 | 372.4 |
| 352 | 364.3 |
| 353 | 358.1 |
| 354 | 314.2 |
| 355 | 314.2 |
| 356 | 308.2 |
| 357 | 292.2 |
| 358 | 294.2 |
| 359 | 292.2 |
| 360 | 291.2 |
| 361 | 277.2 |
| 362 | 286.1 |
| 363 | 280.2 |
| 364 | 270.2 |
| 365 | 266.2 |
| 366 | 400 |
| 370 | 345 |
| 371 | 522.2 |
| 372 | 348.2 |
| 373 | 345.1 |
| 374 | 318 |
| 375 | 328.3 |
| 376 | 334 |
| 377 | 334.2 |
| 378 | 308.2 |
| 379 | 314.1 |

### Example 91: Synthesis of 1-(4-chloro-1H-indol-6-yl)-3-(1-(pyridin-4-yl)ethyl)urea (Compound 497)

### 1. Procedure for 4-chloro-1H-indol-6-amine

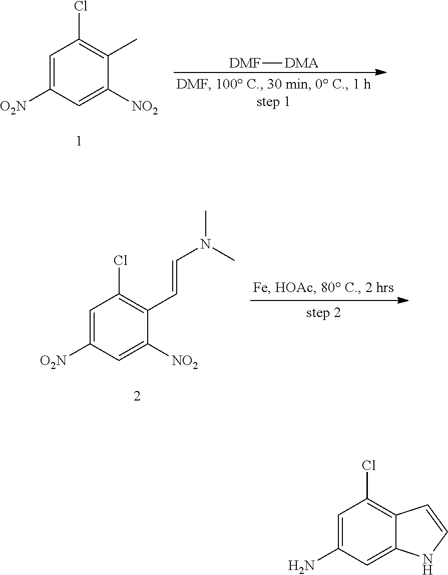

### Step 1: Synthesis of (E)-2-(2-chloro-4,6-dinitrophenyl)-N,N-dimethylethenamine

1-chloro-2-methyl-3,5-dinitro-benzene (8.00 g, 36.94 mmol, 1 equiv.) and DMF-DMA (4.84 g, 40.63 mmol, 5.40 mL, 1.10 equiv.) were dissolved in DMF (120 mL) and the reaction mixture was stirred at 100° C. for 30 mins. Then the reaction mixture was poured into ice-water and stirred for 1 h. The precipitate was filtered and washed with water to give 2-(2-chloro-4,6-dinitro-phenyl)-N,N-dimethyl-ethenamine (6.00 g, 19.88 mmol, 53.81% yield, 90% purity). The crude product was used for the next step without further purification.

### Step 2: Synthesis of 4-chloro-1H-indol-6-amine

2-(2-chloro-4,6-dinitro-phenyl)-N,N-dimethyl-ethenamine (6.00 g, 19.88 mmol, 90°/a purity, 1 equiv.) in AcOH (100 mL) was added Fe (5.55 g, 99.39 mmol, 5 equiv.). The reaction mixture was stirred at 80° C. for 2 hrs. The resulting mixture was filtered to give filtrate, and adjusted to pH 8 by dropwise addition of saturated aqueous Na₂CO₃. The reaction mixture was partitioned between EtOAc 300 mL and H₂O 300 mL. The organic phase was separated, washed with H₂O 150 mL (50 mL*3), dried over by Na₂SO₄, filtered and concentrated under reduced pressure to give crude 4-chloro-1H-indol-6-amine. The residue was purified by flash silica gel chromatography (ISCO®; 40 g SepaFlash® Silica Flash Column, Eluent of 0˜50% Tetrahydrofuran/Petroleum ethergradient @ 60 mL/min) to give 4-chloro-1H-indol-6-amine (1.8 g, 9.72 mmol, 48.92% yield, 90% purity) was obtained as a Brown oil. MS-ESI, 167.1 [M+H⁺].

### 2. Procedure for 1-(4-chloro-1H-indol-6-yl)-3-(1-(pyridin-4-yl)ethyl)urea

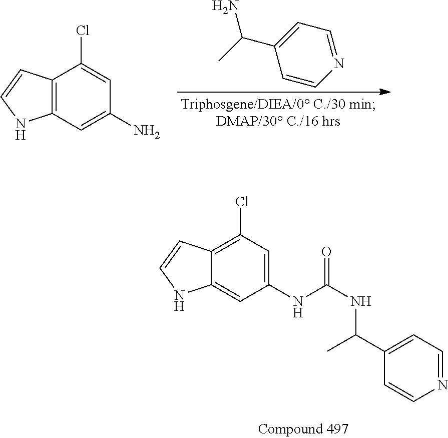

Procedure: Triphosgene (24.42 mg, 82.50 umol, 0.33 equiv.) was dissolved in THF (5 mL) and stirred at 0° C. for 5 mins. Then a solution of 4-chloro-1H-indol-6-amine (41.50 mg, 250 umol, 1.0 equiv.) dissolved in DMF (5 mL) and DIEA (250 μl, 1.72 mmol, 6.0 equiv.) were added dropwise. The reaction mixture was stirred at 0° C. for 30 mins. After that, a solution of 1-(pyridin-4-yl)ethanamine dissolved in DMF (5 mL) and DMAP were added respectively. The reaction mixture was stirred at 30° C. for 16 hrs. The reaction mixture was concentrated by speedvac. The residue was purified by prep HPLC to give 1-(4-chloro-1H-indol-6-yl)-3-(1-(pyridin-4-yl)ethyl)urea (10.42 mg, 0.033 mmol). MS-ESI, 315 [M+H⁺].

¹H NMR (400 MHz, DMSO-d₆) δ ppm 1.39 (d, 3H) 4.81 (quin 1H) 6.26-6.38 (m, 1H) 6.71 (d, 1H) 7.04 (d, 1H) 7.22-7.43 (m, 3H) 7.22-7.43 (m, 1H) 7.53 (s, 1H) 8.16 (s, 1H) 8.45-8.57 (m, 3H) 11.16 (br s, 1H)

The compounds in the table below were prepared using the above procedure. (LC-MS methods BA or BB)

| Example | Com- | | | LC-MS, MS-ESI, |
|---|---|---|---|---|
| # | pound | Structure | IUPAC Name | --[M + H⁺]. |
| 92 | 630 | 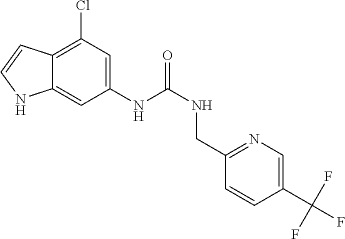 [SMILES: O=C(NCc1ccc(C(F)(F)F)cn1)Nc1cc(Cl)c2cc[nH]c2c1] | 3-(4-chloro-1H- indol-6-yl)-1-{[5- (trifluoromethyl) pyridin-2-yl] methyl}urea | |
| 93 | 496 | 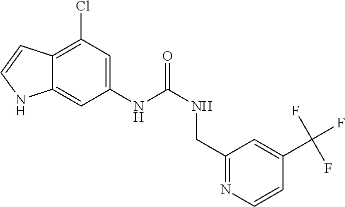 [SMILES: O=C(NCc1cc(C(F)(F)F)ccn1)Nc1cc(Cl)c2cc[nH]c2c1] | 3-(4-chloro-1H- indol-6-yl)-1-{[4- (trifluoromethyl) pyridin-2-yl]methyl} urea | 369 |
| 94 | 495 | 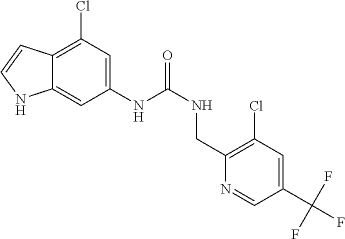 [SMILES: O=C(NCc1ncc(C(F)(F)F)cc1Cl)Nc1cc(Cl)c2cc[nH]c2c1] | 3-(4-chloro-1H- indol-6-yl)-1-{[3- chloro-5- (trifluoromethyl) pyridin-2-yl]methyl} urea | 403 |
| 95 | 631 | 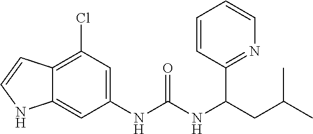 [SMILES: CC(C)CC(NC(=O)Nc1cc(Cl)c2cc[nH]c2c1)c1ccccn1] | 3-(4-chloro-1H- indol-6-yl)-1-[3- methyl-1-(pyridin-2- yl)butyl]urea | 357.1 |
| 96 | 632 | 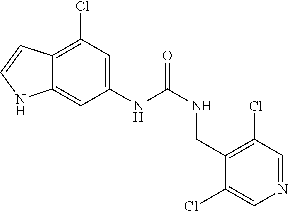 [SMILES: O=C(NCc1c(Cl)cncc1Cl)Nc1cc(Cl)c2cc[nH]c2c1] | 3-(4-chloro-1H- indol-6-yl)-1-[(3,5- dichloropyridin-4 - yl)methyl]urea | 369 |
| 97 | 494 | 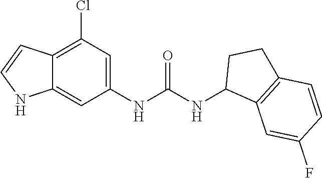 [SMILES: O=C(Nc1cc(Cl)c2cc[nH]c2c1)NC1CCc2ccc(F)cc21] | 3-(4-chloro-1H- indol-6-yl)-1-(6- fluoro-2,3-dihydro- 1H-inden-1-yl)urea | 344.1 |
| 98 | 493 | 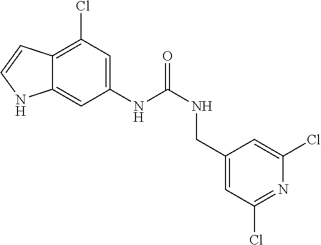 [SMILES: O=C(NCc1cc(Cl)nc(Cl)c1)Nc1cc(Cl)c2cc[nH]c2c1] | 3-(4-chloro-1H- indol-6-yl)-1-[(2,6- dichloropyridin-4- yl)methyl]urea | 369 |
| 99 | 492 | 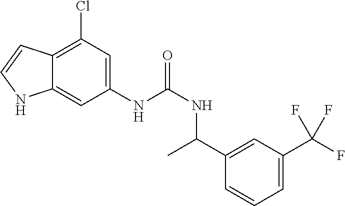 [SMILES: CC(NC(=O)Nc1cc(Cl)c2cc[nH]c2c1)c1cccc(C(F)(F)F)c1] | 3-(4-chloro-1H- indol-6-yl)-1-{1-[3- (trifluoromethyl) phenyl]ethyl}urea | 382.1 |
| 100 | 633 | 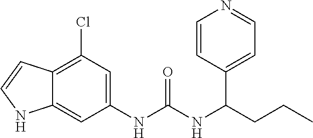 [SMILES: CCCC(NC(=O)Nc1cc(Cl)c2cc[nH]c2c1)c1ccncc1] | 3-(4-chloro-1H- indol-6-yl)-1-[1- (pyridin-4- yl)butyl]urea | 343.2 |
| 101 | 491 | 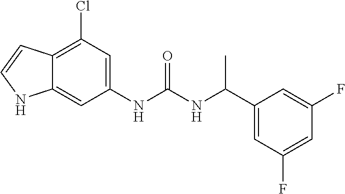 [SMILES: CC(NC(=O)Nc1cc(Cl)c2cc[nH]c2c1)c1cc(F)cc(F)c1] | 3-(4-chloro-1H- indol-6-yl)-1-[1-(3,5- difluorophenyl)ethyl] urea | 350 |
| 102 | 490 | 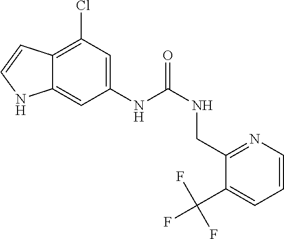 [SMILES: O=C(NCc1ncccc1C(F)(F)F)Nc1cc(Cl)c2cc[nH]c2c1] | 3-(4-chloro-1H- indol-6-yl)-1-{[3- (trifluoromethyl) pyridin-2-yl]methyl} urea | 369 |
| 103 | 635 | 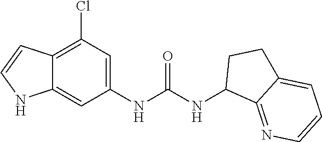 [SMILES: O=C(Nc1cc(Cl)c2cc[nH]c2c1)NC1CCc2cccnc21] | 3-(4-chloro-1H- indol-6-yl)-1- {5H,6H,7H- cyclopenta[b] pyridin-7-yl}urea | 327.2 |
| 104 | 489 | 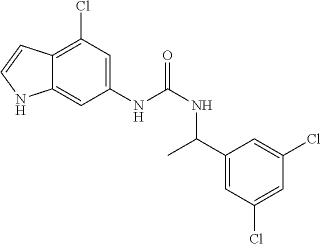 [SMILES: CC(NC(=O)Nc1cc(Cl)c2cc[nH]c2c1)c1cc(Cl)cc(Cl)c1] | 3-(4-chloro-1H- indol-6-yl)-1-[1-(3,5- dichlorophenyl) ethyl]urea | 381.8 |
| 105 | 488 | 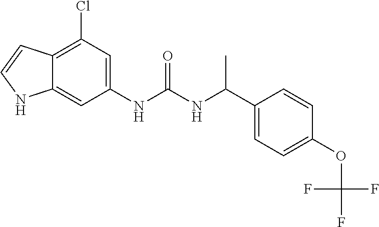 [SMILES: CC(NC(=O)Nc1cc(Cl)c2cc[nH]c2c1)c1ccc(OC(F)(F)F)cc1] | 3-(4-chloro-1H- indol-6-yl)-1-{1-[4- (trifluoromethoxy) phenyl]ethyl}urea | 398.1 |
| 106 | 635 | 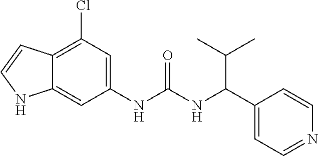 [SMILES: CC(C)C(NC(=O)Nc1cc(Cl)c2cc[nH]c2c1)c1ccncc1] | 3-(4-chloro-1H- indol-6-yl)-1-[2- methyl-1-(pyridin-4- yl)propyl]urea | |
| 107 | 636 | 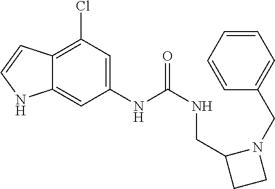 [SMILES: O=C(NCC1CCN1Cc1ccccc1)Nc1cc(Cl)c2cc[nH]c2c1] | 3-[(1-benzylazetidin- 2-yl)methyl]-1-(4- chloro-1H-indol-6- yl)urea | |
| 108 | 487 | 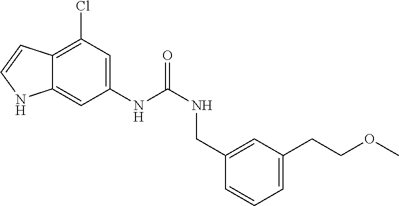 [SMILES: COCCc1cccc(CNC(=O)Nc2cc(Cl)c3cc[nH]c3c2)c1] | 3-(4-chloro-1H- indol-6-yl)-1-{[3-(2- methoxyethyl) phenyl]methyl}urea | 358.1 |
| 109 | 486 | 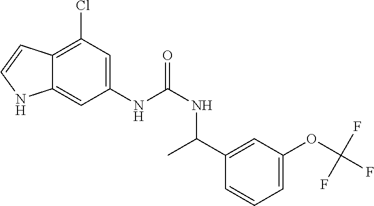 [SMILES: CC(NC(=O)Nc1cc(Cl)c2cc[nH]c2c1)c1cccc(OC(F)(F)F)c1] | 3-(4-chloro-1H- indol-6-yl)-1-{1-[3- (trifluoromethoxy) phenyl]ethyl}urea | 398.1 |
| 110 | 637 | 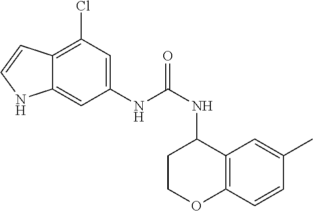 [SMILES: Cc1ccc2c(c1)C(NC(=O)Nc1cc(Cl)c3cc[nH]c3c1)CCO2] | 3-(4-chloro-1H- indol-6-yl)-1-(6- methyl-3,4-dihydro- 2H-1-benzopyran-4- yl)urea | 356.1 |
| 111 | 502 | 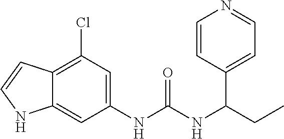 [SMILES: CCC(NC(=O)Nc1cc(Cl)c2cc[nH]c2c1)c1ccncc1] | 3-(4-chloro-1H- indol-6-yl)-1-[1- (pyridin-4- yl)propyl]urea | 329 |
| 112 | 485 | 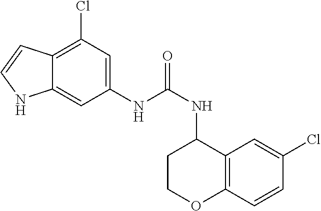 [SMILES: O=C(Nc1cc(Cl)c2cc[nH]c2c1)NC1CCOc2ccc(Cl)cc21] | 3-(4-chloro-1H- indol-6-yl)-1-(6- chloro-3,4-dihydro- 2H-1-benzopyran-4- yl)urea | 376 |
| 113 | 638 | 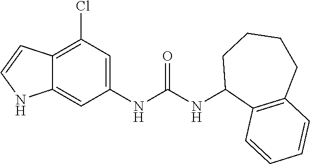 [SMILES: O=C(Nc1cc(Cl)c2cc[nH]c2c1)NC1CCCCc2ccccc21] | 3-(4-chloro-1H- indol-6-yl)-1- {6,7,8,9-tetrahydro- 5H-benzo[7]annulen- 5-yl}urea | 354.1 |
| 114 | 639 | 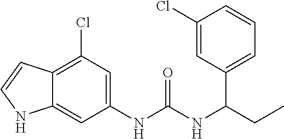 [SMILES: CCC(NC(=O)Nc1cc(Cl)c2cc[nH]c2c1)c1cccc(Cl)c1] | 3-(4-chloro-1H- indol-6-yl)-1-[1-(3- chlorophenyl)propyl] urea | 362.1 |
| 115 | 640 | 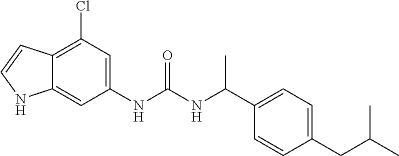 [SMILES: CC(C)Cc1ccc(C(C)NC(=O)Nc2cc(Cl)c3cc[nH]c3c2)cc1] | 3-(4-chloro-1H- indol-6-yl)-1-{1-[4- (2-methylpropyl) phenyl]ethyl}urea | |
| 116 | 484 | 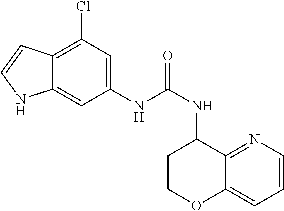 [SMILES: O=C(Nc1cc(Cl)c2cc[nH]c2c1)NC1CCOc2cccnc21] | 3-(4-chloro-1H- indol-6-yl)-1- {2H,3H,4H- pyrano[3,2-b] pyridin-4-yl}urea | 343 |
| 117 | 641 | 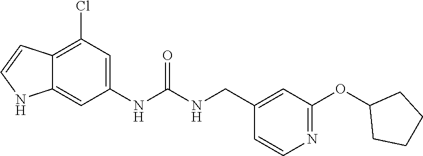 [SMILES: O=C(NCc1ccnc(OC2CCCC2)c1)Nc1cc(Cl)c2cc[nH]c2c1] | 3-(4-chloro-1H- indol-6-yl)-1-{[2- (cyclopentyloxy) pyridin-4-yl] methyl}urea | |
| 118 | 483 | 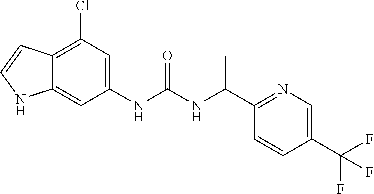 [SMILES: CC(NC(=O)Nc1cc(Cl)c2cc[nH]c2c1)c1ccc(C(F)(F)F)cn1] | 3-(4-chloro-1H- indol-6-yl)-1-{1-[5- (trifluoromethyl) pyridin-2-yl] ethyl}urea | 383.1 |
| 119 | 482 | 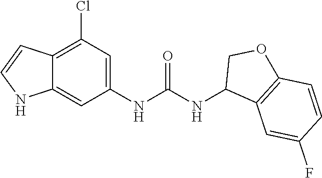 [SMILES: O=C(Nc1cc(Cl)c2cc[nH]c2c1)NC1COc2ccc(F)cc21] | 3-(4-chloro-1H- indol-6-yl)-1-(5- fluoro-2,3-dihydro-1- benzofuran-3-yl)urea | 346.1 |
| 120 | 481 | 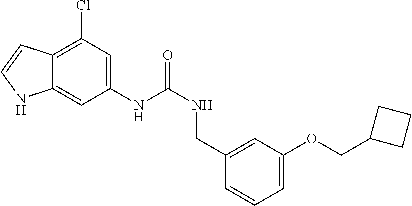 [SMILES: O=C(NCc1cccc(OCC2CCC2)c1)Nc1cc(Cl)c2cc[nH]c2c1] | 3-(4-chloro-1H- indol-6-yl)-1-{[3- (cyclobutylmethoxy) phenyl]methyl}urea | 384.2 |
| 121 | 642 | 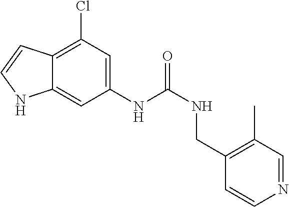 [SMILES: Cc1cnccc1CNC(=O)Nc1cc(Cl)c2cc[nH]c2c1] | 3-(4-chloro-1H- indol-6-yl)-1-[(3- methylpyridin-4- yl)methyl]urea | 315.2 |
| 122 | 643 | 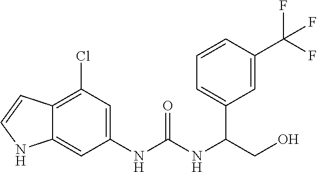 [SMILES: O=C(Nc1cc(Cl)c2cc[nH]c2c1)NC(CO)c1cccc(C(F)(F)F)c1] | 3-(4-chloro-1H- indol-6-yl)-1-{2- hydroxy-1-[3- (trifluoromethyl) phenyl]ethyl}urea | |
| 123 | 644 | 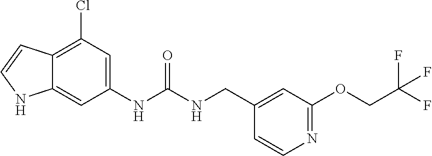 [SMILES: O=C(NCc1ccnc(OCC(F)(F)F)c1)Nc1cc(Cl)c2cc[nH]c2c1] | 3-(4-chloro-1H- indol-6-yl)-1-{[2- (2,2,2-trifluoroethoxy) pyridin-4-yl]methyl} urea | |
| 124 | 480 | 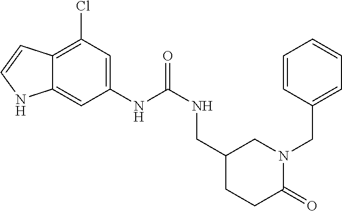 [SMILES: O=C(NCC1CCC(=O)N(Cc2ccccc2)C1)Nc1cc(Cl)c2cc[nH]c2c1] | 1-[(1-benzyl-6- oxopiperidin-3- yl)methyl]-3-(4- chloro-1H-indol-6- yl)urea | 410.9 |
| 125 | 645 | 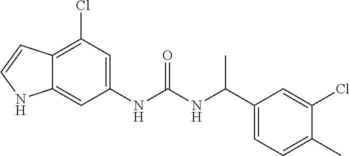 [SMILES: Cc1ccc(C(C)NC(=O)Nc2cc(Cl)c3cc[nH]c3c2)cc1Cl] | 3-(4-chloro-1H- indol-6-yl)-1-[1-(3- chloro-4- methylphenyl)ethyl] urea | 362 |
| 126 | 479 | 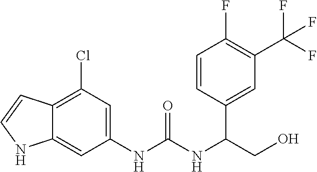 [SMILES: O=C(Nc1cc(Cl)c2cc[nH]c2c1)NC(CO)c1ccc(F)c(C(F)(F)F)c1] | 3-(4-chloro-1H- indol-6-yl)-1-{1-[4- fluoro-3- (trifluoromethyl) phenyl]-2- hydroxyethyl}urea | 416.1 |
| 127 | 646 | 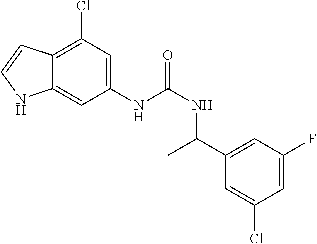 [SMILES: CC(NC(=O)Nc1cc(Cl)c2cc[nH]c2c1)c1cc(F)cc(Cl)c1] | 3-(4-chloro-1H- indol-6-yl)-1-[1-(3- chloro-5- fluorophenyl)ethyl] urea | 366 |
| 128 | 647 | 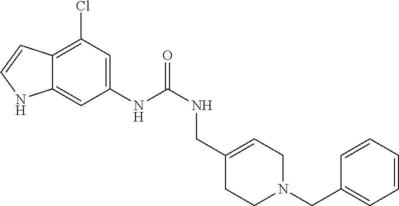 [SMILES: O=C(NCC1=CCN(Cc2ccccc2)CC1)Nc1cc(Cl)c2cc[nH]c2c1] | 1-[(1-benzyl-1,2,3,6- tetrahydropyridin-4- yl)methyl]-3-(4- chloro-1H-indol-6- yl)urea | 395.2 |
| 129 | 648 | 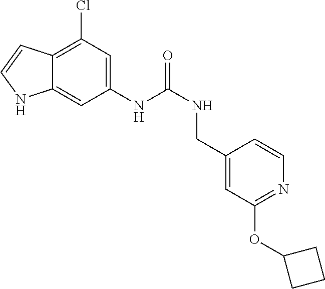 [SMILES: O=C(NCc1ccnc(OC2CCC2)c1)Nc1cc(Cl)c2cc[nH]c2c1] | 3-(4-chloro-1H- indol-6-yl)-1-[(2- cyclobutoxypyridin- 4-yl)methyl]urea | |
| 130 | 649 | 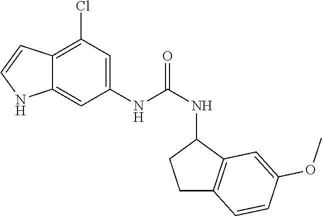 [SMILES: COc1ccc2c(c1)C(NC(=O)Nc1cc(Cl)c3cc[nH]c3c1)CC2] | 3-(4-chloro-1H- indol-6-yl)-1-(6- methoxy-2,3- dihydro-1H-inden-1- yl)urea | 356.1 |
| 131 | 650 | 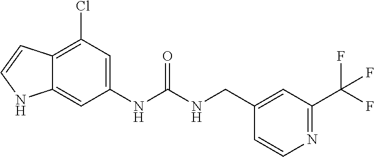 [SMILES: O=C(NCc1ccnc(C(F)(F)F)c1)Nc1cc(Cl)c2cc[nH]c2c1] | 3-(4-chloro-1H- indol-6-yl)-1-{[2- (trifluoromethyl) pyridin-4-yl]methyl} urea | |
| 132 | 651 | 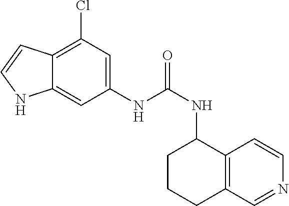 [SMILES: O=C(Nc1cc(Cl)c2cc[nH]c2c1)NC1CCCc2cnccc21] | 3-(4-chloro-1H- indol-6-yl)-1- (5,6,7,8- tetrahydroisoquinolin- 5-yl)urea | 341.2 |
| 133 | 652 | 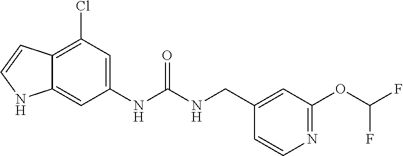 [SMILES: O=C(NCc1ccnc(OC(F)F)c1)Nc1cc(Cl)c2cc[nH]c2c1] | 3-(4-chloro-1H- indol-6-yl)-1-{[2- (difluoromethoxy) pyridin-4- yl]methyl}urea | |

### Example 134: 1-(4-chloro-1H-indol-6-yl)-3-((2-chloro-6-methylpyridin-4-yl)methyl)urea (Compound 611)

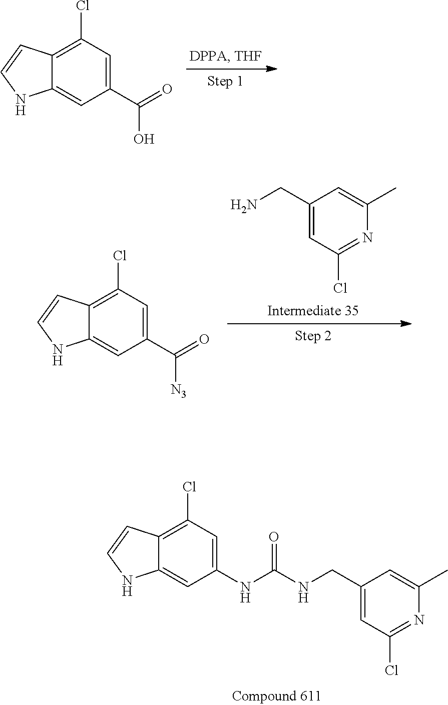

### Step 1: 4-chloro-1H-indole-6-carbonyl azide

4-Chloro-1H-indole-6-carboxylic acid (305.0 mg, 1.6 mmol, 1.0 equiv.) was dissolved in THF (12.0 mL), DPPA (643.7 mg, 2.3 mmol, 1.5 equiv.) and TEA (0.4 mL, 3.1 mmol, 2.0 equiv.) were added at 0° C. The resulting mixture was stirred for overnight at room temperature under atmosphere of nitrogen and concentrated under vacuum to give crude 4-chloro-1H-indole-6-carbonyl azide (762 mg) as a yellow solid, which was used to next step directly.

### Step 2: 1-(4-chloro-1H-indol-6-yl)-3-((2-chloro-6-methylpyridin-4-yl)methyl)urea

1-(2-chloro-6-methylpyridin-4-yl)methanamine (200.0 mg, 1.3 mmol, 1.0 equiv.) was dissolved in toluene (20.0 mL), TEA (0.4 mL, 2.6 mmol, 2.0 equiv.), 4-chloro-1H-indole-6-carbonyl azide (281.7 mg, 1.3 mmol, 1.0 equiv.) were added. The resulting solution was stirred for 6 hours at 100° C. and concentrated under vacuum. The residue was purified by flash column chromatography on silica gel, eluting with ethyl acetate/petroleum ether (1:1) to give the crude product, which was further purified by Prep-HPLC with the following conditions: Column: YMC-Actus Triart C18, 30*250, Sum; Mobile Phase A: Water (10 MMOL/L NH₄HCO₃), Mobile Phase B: ACN; Flow rate: 60 mL/min; Gradient: 40 B to 50 B in 8 min; 254/220 nm; RTL: 7.5. This resulted in 3-(4-chloro-1H-indol-6-yl)-1-[(2-chloro-6-methylpyridin-4-yl)methyl]urea (70 mg, 15.7%) as an off-white solid. LCMS Method CH: [M+H]⁺=349. ¹H NMR (400 MHz, DMSO-d₆): δ 11.19 (s, 1H), 8.74 (s, 1H), 7.59 (s, 1H), 7.31-7.29 (m, 1H), 7.20-7.19 (m, 2H), 7.10 (s, 1H), 6.72 (t, 1H), 6.34 (d, 1H), 4.31 (d, 2H), 2.44 (s, 3H).

The analogs prepared in the following table were prepared using the same method described for Example 134.

| Compound | Starting materials Used | Structure | LCMS data |
|---|---|---|---|
| Example 135 (Compound 612) | 4-chloro-1H-indole-6-carboxylic acid; Intermediate A1 | 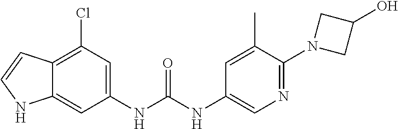 [SMILES: Cc1cc(NC(=O)Nc2cc(Cl)c3cc[nH]c3c2)cnc1N1CC(O)C1] | Method CE: MS-ESI: 372 [M + H]⁺. |
| Example 136 (Compound 487) | 4-chloro-1H-indole-6-carboxylic acid; Intermediate A2 | 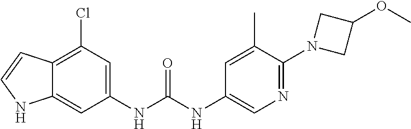 [SMILES: COC1CN(c2ncc(NC(=O)Nc3cc(Cl)c4cc[nH]c4c3)cc2C)C1] | Method CI: MS-ESI: 386 [M + H]⁺. |
| Example 137 (Compound 613) | 4-chloro-1H-indole-6-carboxylic acid; Intermediate A9 | 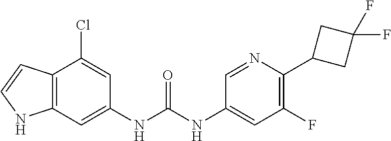 [SMILES: O=C(Nc1cnc(C2CC(F)(F)C2)c(F)c1)Nc1cc(Cl)c2cc[nH]c2c1] | Method CK: MS-ESI: 395 [M + H]⁺. |
| Example 138 (Compound 614) | 4-chloro-1H-indole-6-carboxylic acid; Intermediate A3 | 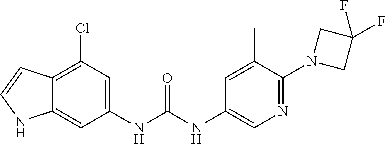 [SMILES: Cc1cc(NC(=O)Nc2cc(Cl)c3cc[nH]c3c2)cnc1N1CC(F)(F)C1] | Method CE: MS-ESI: 392 [M + H]⁺. |
| Example 139 (Compound 500) | 4-chloro-1H-indole-6-carboxylic acid; Intermediate A4 | 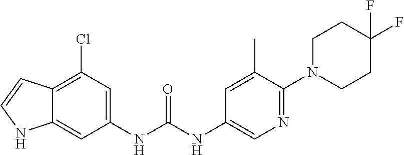 [SMILES: Cc1cc(NC(=O)Nc2cc(Cl)c3cc[nH]c3c2)cnc1N1CCC(F)(F)CC1] | Method CC: MS-ESI: 420 [M + H]⁺. |
| Example 140 (Compound 516) | 4-chloro-1H-indole-6-carboxylic acid; Intermediate A7 | 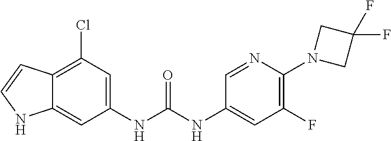 [SMILES: O=C(Nc1cnc(N2CC(F)(F)C2)c(F)c1)Nc1cc(Cl)c2cc[nH]c2c1] | Method CI: MS-ESI: 396 [M + H]⁺. |
| Example 141 (Compound 537) | 4-chloro-1H-indole-6-carboxylic acid; 1-(3-chloro-5- (trifluoromethyl)phenyl)ethan-1- amine | 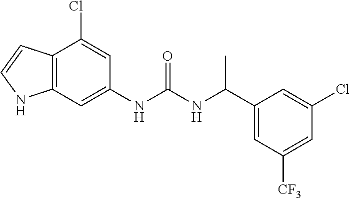 [SMILES: CC(NC(=O)Nc1cc(Cl)c2cc[nH]c2c1)c1cc(Cl)cc(C(F)(F)F)c1] | Method CL: MS-ESI: 416 [M + H]⁺. |
| Example 142 (Compound 536) | 4-chloro-1H-indole-6-carboxylic acid; (3-chloro-5- (trifluoromethyl)phenyl)methanamine | 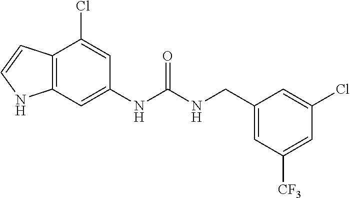 [SMILES: O=C(NCc1cc(Cl)cc(C(F)(F)F)c1)Nc1cc(Cl)c2cc[nH]c2c1] | Method CL: MS-ESI: 402 [M + H]⁺. |
| Example 143 (Compound 542) | 4-chloro-1H-indole-6-carboxylic acid; Intermediate A12 | 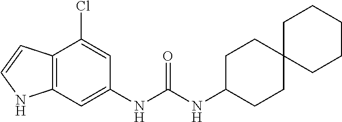 [SMILES: O=C(Nc1cc(Cl)c2cc[nH]c2c1)NC1CCC2(CCCCC2)CC1] | Method CI: MS-ESI: 360 [M + H]⁺. |
| Example 144 (Compound 548) | 4-chloro-1H-indole-6-carboxylic acid; Intermediate A13 | 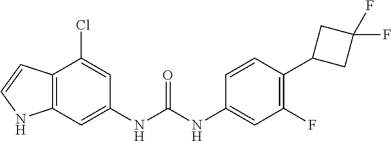 [SMILES: O=C(Nc1ccc(C2CC(F)(F)C2)c(F)c1)Nc1cc(Cl)c2cc[nH]c2c1] | Method CI: MS-ESI: 394 [M + H]⁺. |
| Example 145 (Compound 251) | 4-chloro-1H-indole-6-carboxylic acid; 4-(trifluoromethyl)aniline | 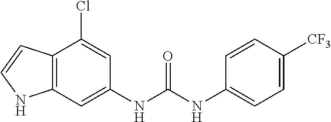 [SMILES: O=C(Nc1ccc(C(F)(F)F)cc1)Nc1cc(Cl)c2cc[nH]c2c1] | Method CI: MS-ESI: 354 [M + H]⁺. |
| Example 146 (Compound 499) | Intermediate A30; Intermediate A4 | 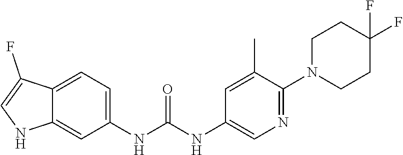 [SMILES: Cc1cc(NC(=O)Nc2ccc3c(F)c[nH]c3c2)cnc1N1CCC(F)(F)CC1] | Method CC: MS-ESI: 404 [M + H]⁺. |
| Example 147 (Compound 514) | Intermediate A30; Intermediate A5 | 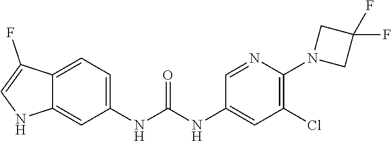 [SMILES: O=C(Nc1cnc(N2CC(F)(F)C2)c(Cl)c1)Nc1ccc2c(F)c[nH]c2c1] | Method CK: MS-ESI: 396 [M + H]⁺. |
| Example 148 (Compound 515) | Intermediate A30; Intermediate A6 | 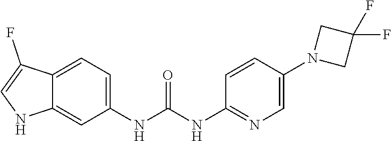 [SMILES: O=C(Nc1ccc2c(F)c[nH]c2c1)Nc1ccc(N2CC(F)(F)C2)cn1] | Method CE: MS-ESI: 362 [M + H]⁺. |
| Example 149 (Compound 517) | Intermediate A30; Intermediate A8 | 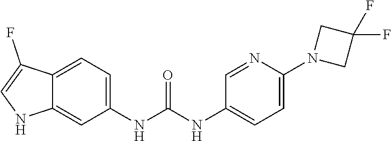 [SMILES: O=C(Nc1ccc(N2CC(F)(F)C2)nc1)Nc1ccc2c(F)c[nH]c2c1] | Method CE: MS-ESI: 362 [M + H]⁺. |
| Example 150 (Compound 523) | Intermediate A30; 3-(trifluoromethyl)aniline | 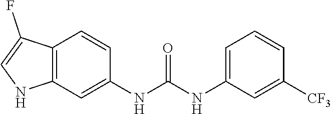 [SMILES: O=C(Nc1cccc(C(F)(F)F)c1)Nc1ccc2c(F)c[nH]c2c1] | Method CK: MS-ESI: 338 [M + H]⁺. |
| Example 151 (Compound 531) | Intermediate A30; Intermediate A7 | 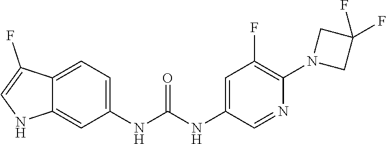 [SMILES: O=C(Nc1cnc(N2CC(F)(F)C2)c(F)c1)Nc1ccc2c(F)c[nH]c2c1] | Method CE: MS-ESI: 380 [M + H]⁺. |
| Example 152 (Compound 535) | Intermediate A30; 3-fluoro-5-(trifluoromethyl)aniline | 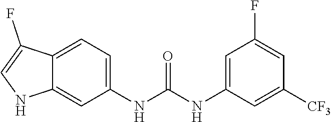 [SMILES: O=C(Nc1cc(F)cc(C(F)(F)F)c1)Nc1ccc2c(F)c[nH]c2c1] | Method CK: MS-ESI: 356 [M + H]⁺. |
| Example 153 (Compound 538) | Intermediate A30; (3-chloro-5- (trifluoromethyl)phenyl)methanamine | 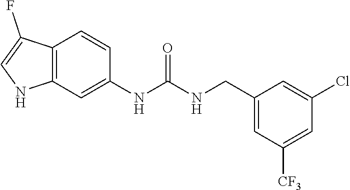 [SMILES: O=C(NCc1cc(Cl)cc(C(F)(F)F)c1)Nc1ccc2c(F)c[nH]c2c1] | Method CE: MS-ESI: 386 [M + H]⁺. |
| Example 154 (Compound 242) | Intermediate A30; 4-(trifluoromethyl)aniline | 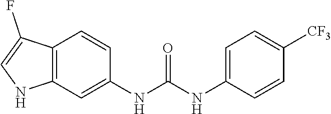 [SMILES: O=C(Nc1ccc(C(F)(F)F)cc1)Nc1ccc2c(F)c[nH]c2c1] | Method CE: MS-ESI: 338 [M + H]⁺. |
| Example 155 (Compound 503) | 4-methoxy-1H-indole-6-carboxylic acid; (3-chloro-5- (trifluoromethyl)phenyl)methanamine | 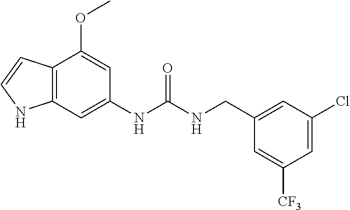 [SMILES: COc1cc(NC(=O)NCc2cc(Cl)cc(C(F)(F)F)c2)cc2[nH]ccc12] | Method CE: MS-ESI: 398 [M + H]⁺. |
| Example 156 (Compound 504) | 4-methoxy-1H-indole-6-carboxylic acid; 4-(trifluoromethyl)aniline | 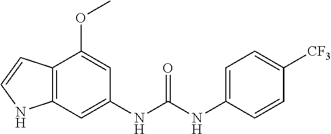 [SMILES: COc1cc(NC(=O)Nc2ccc(C(F)(F)F)cc2)cc2[nH]ccc12] | Method CE: MS-ESI: 350 [M + H]⁺. |
| Example 157 (Compound 511) | 4-cyano-1H-indole-6-carboxylic acid; Intermediate A7 | 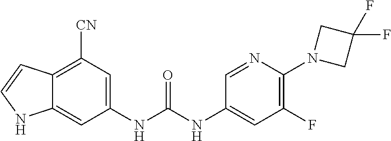 [SMILES: N#Cc1cc(NC(=O)Nc2cnc(N3CC(F)(F)C3)c(F)c2)cc2[nH]ccc12] | Method CE: MS-ESI: 387 [M + H]⁺. |
| Example 158 (Compound 518) | Intermediate A31; 4-(trifluoromethyl)aniline | 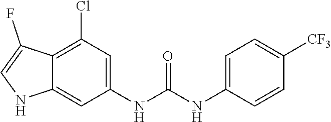 [SMILES: O=C(Nc1ccc(C(F)(F)F)cc1)Nc1cc(Cl)c2c(F)c[nH]c2c1] | Method CE: MS-ESI: 372 [M + H]⁺. |
| Example 159 (Compound 522) | 3-acetyl-1H-indole-6-carboxylic acid; 4-(trifluoromethyl)aniline | 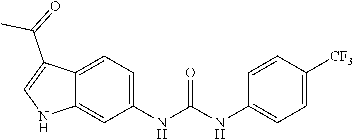 [SMILES: CC(=O)c1c[nH]c2cc(NC(=O)Nc3ccc(C(F)(F)F)cc3)ccc12] | Method CE: MS-ESI: 362 [M + H]⁺. |
| Example 160 (Compound 524) | 1H-indole-6-carboxylic acid; Intermediate A10 | 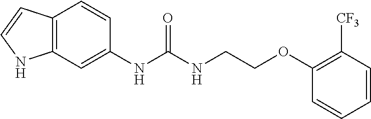 [SMILES: O=C(NCCOc1ccccc1C(F)(F)F)Nc1ccc2cc[nH]c2c1] | Method CD: MS-ESI: 364 [M + H]⁺. |
| Example 161 (Compound 525) | 1H-indole-6-carboxylic acid; Intermediate A11 | 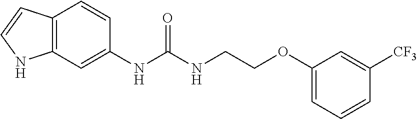 [SMILES: O=C(NCCOc1cccc(C(F)(F)F)c1)Nc1ccc2cc[nH]c2c1] | Method CE: MS-ESI: 364 [M + H]⁺. |
| Example 162 (Compound 572) | Intermediate A32; 4-(trifluoromethyl)aniline | 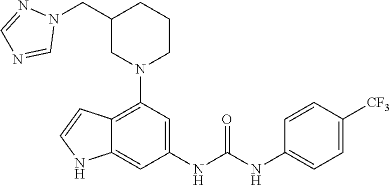 [SMILES: O=C(Nc1ccc(C(F)(F)F)cc1)Nc1cc(N2CCCC(Cn3cncn3)C2)c2cc[nH]c2c1] | Method CK: MS-ESI: 484 [M + H]⁺. |

### Example 163: cyclopentyl 6-(3-(4-(trifluoromethyl)phenyl)ureido)-1H-indole-4-carboxylate (Compound 602)

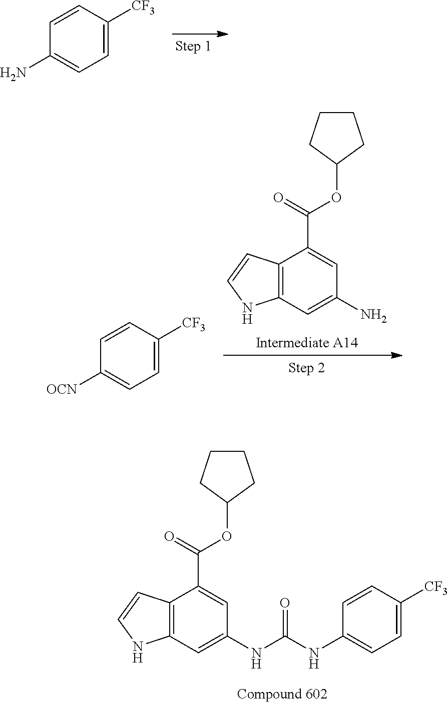

### Step 1: 1-isocyanato-4-(trifluoromethyl)benzene

P-trifluoromethylaniline (300.0 mg, 1.8 mmol, 1.0 equiv.) was dissolved in THF (10.0 mL), triphosgene (254.1 mg, 0.3 mmol, 0.5 equiv.) was added at 0° C. The resulting solution was stirred for 2 hours at 70° C. and then concentrated under vacuum to give 1-isocyanato-4-(trifluoromethyl)benzene (240 mg, 68.9%) as a brown yellow solid.

### Step 2: cyclopentyl 6-(3-(4-(trifluoromethyl)phenyl)ureido)-1H-indole-4-carboxylate

6-Amino-1H-indole-4-carboxylate (240.0 mg, 1.0 mmol, 1.0 equiv.) was dissolved in THF (10.0 mL), 1-isocyanato-4-(trifluoromethyl)benzene (183.8 mg, 1.0 mmol, 1.0 equiv.) and TEA (0.3 mL, 2.0 mmol, 2.0 equiv.) were added under atmosphere of nitrogen. The resulting mixture was stirred for 3 hours at room temperature and concentrated under vacuum. The residue was purified by Prep-HPLC with the following conditions: Column: YMC-Actus Triart C18, 30*250, Sum; Mobile Phase A: Water (10 MMOL/L NH₄HCO₃), Mobile Phase B: ACN; Flow rate: 60 mL/min; Gradient: 40 B to 50 B in 8 min; 254/220 nm. This resulted in cyclopentyl 6-([[4-(trifluoromethyl)phenyl]carbamoyl] amino)-1H-indole-4-carboxylate (180 mg, 42.5%) as a white solid. LCMS Method CE: [M+H]⁺=432.

¹H NMR (400 MHz, DMSO-d₆) δ 11.31 (s, 1H), 9.00 (s, 1H), 8.94 (s, 1H), 8.07 (s, 1H), 7.70-7.63 (m, 5H), 7.44 (t, 1H), 6.82 (t, 1H), 5.41-5.39 (m, 1H), 1.99-1.96 (m, 2H), 1.84-1.80 (m, 4H), 1.78-1.75 (m, 2H).

The analogs prepared in the following table were prepared using the same method described for Example 163.

| Compound | Starting materials Used | Structure | LCMS data |
|---|---|---|---|
| Example 164 (Compound 505) | Intermediate A16; 4-(trifluoromethyl)aniline | 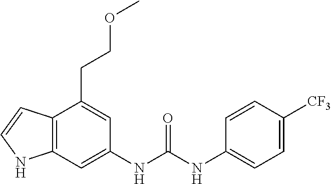 [SMILES: COCCc1cc(NC(=O)Nc2ccc(C(F)(F)F)cc2)cc2[nH]ccc12] | Method CI: MS-ESI: 378 [M + H]⁺. |
| Example 165 (Compound 508) | Intermediate A19; 4-(trifluoromethyl)aniline | 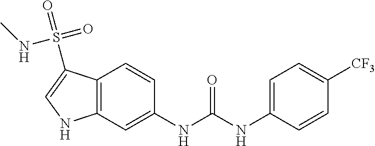 [SMILES: CNS(=O)(=O)c1c[nH]c2cc(NC(=O)Nc3ccc(C(F)(F)F)cc3)ccc12] | Method CK: MS-ESI: 413 [M + H]⁺. |
| Example 166 (Compound 512) | Intermediate A17; 4-(trifluoromethyl)aniline | 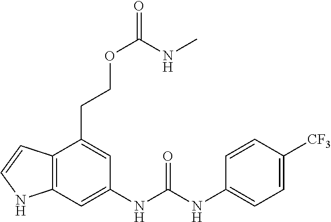 [SMILES: CNC(=O)OCCc1cc(NC(=O)Nc2ccc(C(F)(F)F)cc2)cc2[nH]ccc12] | Method CE: MS-ESI: 421 [M + H]⁺. |
| Example 167 (Compound 513) | Intermediate A18; 4-(trifluoromethyl)aniline | 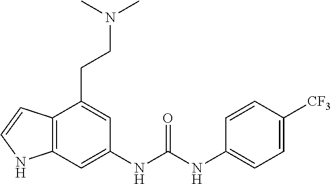 [SMILES: CN(C)CCc1cc(NC(=O)Nc2ccc(C(F)(F)F)cc2)cc2[nH]ccc12] | Method CI: MS-ESI: 391 [M + H]⁺. |
| Example 168 (Compound 526) | Intermediate A20; 4-(trifluoromethyl)aniline | 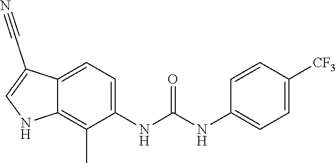 [SMILES: Cc1c(NC(=O)Nc2ccc(C(F)(F)F)cc2)ccc2c(C#N)c[nH]c12] | Method CE: MS-ESI: 359 [M + H]⁺. |
| Example 169 (Compound 527) | Intermediate A22; 4-(trifluoromethyl)aniline | 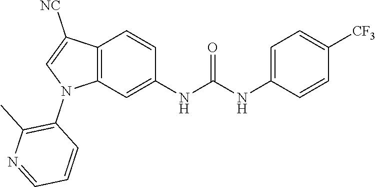 [SMILES: Cc1ncccc1-n1cc(C#N)c2ccc(NC(=O)Nc3ccc(C(F)(F)F)cc3)cc21] | Method CK: MS-ESI: 436 [M + H]⁺. |
| Example 170 (Compound 528) | Intermediate A21; 4-(trifluoromethyl)aniline | 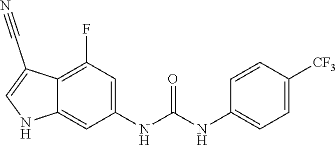 [SMILES: N#Cc1c[nH]c2cc(NC(=O)Nc3ccc(C(F)(F)F)cc3)cc(F)c12] | Method CI: MS-ESI: 363 [M + H]⁺. |
| Example 171 (Compound 529) | Intermediate A23; 4-(trifluoromethyl)aniline | 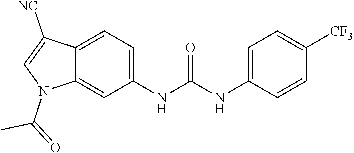 [SMILES: CC(=O)n1cc(C#N)c2ccc(NC(=O)Nc3ccc(C(F)(F)F)cc3)cc21] | Method CE: MS-ESI: 387 [M + H]⁺. |
| Example 172 (Compound 533) | Intermediate A24; 4-(trifluoromethyl)aniline | 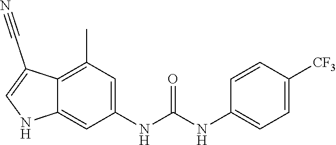 [SMILES: Cc1cc(NC(=O)Nc2ccc(C(F)(F)F)cc2)cc2[nH]cc(C#N)c12] | Method CE: MS-ESI: 359 [M + H]⁺. |
| Example 173 (Compound 540) | Intermediate A25; 4-(trifluoromethyl)aniline | 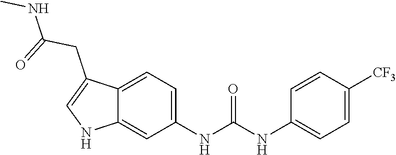 [SMILES: CNC(=O)Cc1c[nH]c2cc(NC(=O)Nc3ccc(C(F)(F)F)cc3)ccc12] | Method CE: MS-ESI: 391 [M + H]⁺. |
| Example 174 (Compound 541) | Intermediate A26; 4-(trifluoromethyl)aniline | 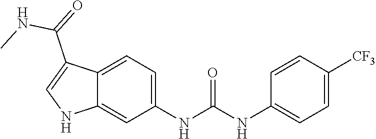 [SMILES: CNC(=O)c1c[nH]c2cc(NC(=O)Nc3ccc(C(F)(F)F)cc3)ccc12] | Method CE: MS-ESI: 377 [M + H]⁺. |
| Example 175 (Compound 543) | Intermediate A27; 4-(trifluoromethyl)aniline | 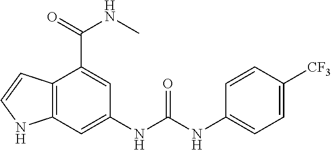 [SMILES: CNC(=O)c1cc(NC(=O)Nc2ccc(C(F)(F)F)cc2)cc2[nH]ccc12] | Method CI: MS-ESI: 377 [M + H]⁺. |
| Example 176 (Compound 544) | Intermediate A28; 4-(trifluoromethyl)aniline | 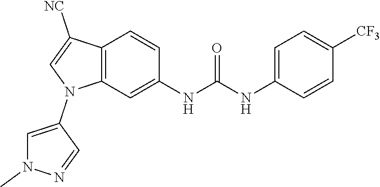 [SMILES: Cn1cc(-n2cc(C#N)c3ccc(NC(=O)Nc4ccc(C(F)(F)F)cc4)cc32)cn1] | Method CI: MS-ESI: 425 [M + H]⁺. |
| Example 177 (Compound 545) | Intermediate A29; 4-(trifluoromethyl)aniline | 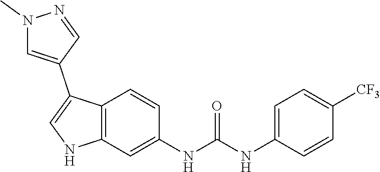 [SMILES: Cn1cc(-c2c[nH]c3cc(NC(=O)Nc4ccc(C(F)(F)F)cc4)ccc23)cn1] | Method CE: MS-ESI: 400 [M + H]⁺. |
| Example 178 (Compound 615) | 6-amino-1H-indole-3-carbonitrile; 4-(trifluoromethyl)aniline | 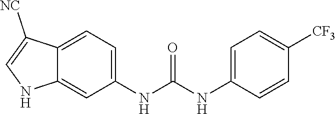 [SMILES: N#Cc1c[nH]c2cc(NC(=O)Nc3ccc(C(F)(F)F)cc3)ccc12] | Method CE: MS-ESI: 345 [M + H]⁺. |
| Example 179 (Compound 539) | 6-amino-1H-indole-3-carbonitrile; 3-(trifluoromethyl)aniline | 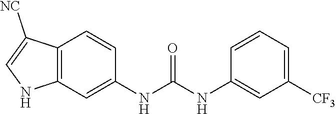 [SMILES: N#Cc1c[nH]c2cc(NC(=O)Nc3cccc(C(F)(F)F)c3)ccc12] | Method CE: MS-ESI: 345 [M + H]⁺. |
| Example 180 (Compound 530) | Intermediate A7; 6-amino-1H-indole-3-carbonitrile | 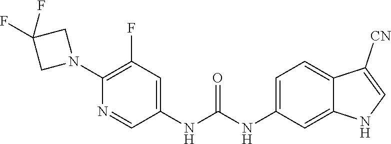 [SMILES: N#Cc1c[nH]c2cc(NC(=O)Nc3cnc(N4CC(F)(F)C4)c(F)c3)ccc12] | Method CC: MS-ESI: 387 [M + H]⁺. |
| Example 181 (Compound 532) | (3-chloro-5- (trifluoromethyl)phenyl)methanamine; 6-amino-1H-indole-3-carbonitrile | 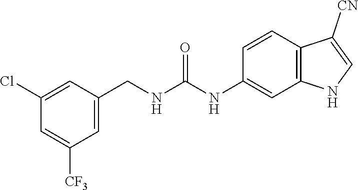 [SMILES: N#Cc1c[nH]c2cc(NC(=O)NCc3cc(Cl)cc(C(F)(F)F)c3)ccc12] | Method CI: MS-ESI: 393 [M + H]⁺. |
| Example 182 (Compound 519) | 2-(pyridin-3-yloxy)ethan-1-amine; 1H-indol-6-amine | 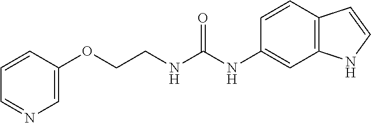 [SMILES: O=C(NCCOc1cccnc1)Nc1ccc2cc[nH]c2c1] | Method CI: MS-ESI: 297 [M + H]⁺. |
| Example 183 (Compound 547) | Intermediate A33; 4-(trifluoromethyl)aniline | 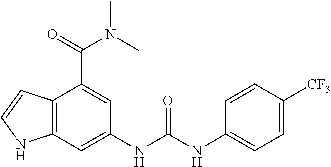 [SMILES: CN(C)C(=O)c1cc(NC(=O)Nc2ccc(C(F)(F)F)cc2)cc2[nH]ccc12] | Method CI: MS-ESI: 391 [M + H]⁺. |
| Example 184 (Compound 546) | Intermediate A34; 4-(trifluoromethyl)aniline | 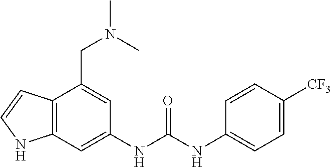 [SMILES: CN(C)Cc1cc(NC(=O)Nc2ccc(C(F)(F)F)cc2)cc2[nH]ccc12] | Method CE: MS-ESI: 377 [M + H]⁺. |

### Example 185: 1-(4-(2-hydroxyethyl)-1H-indol-6-yl)-3-(3-(trifluoromethyl)phenyl)urea (Compound 509)

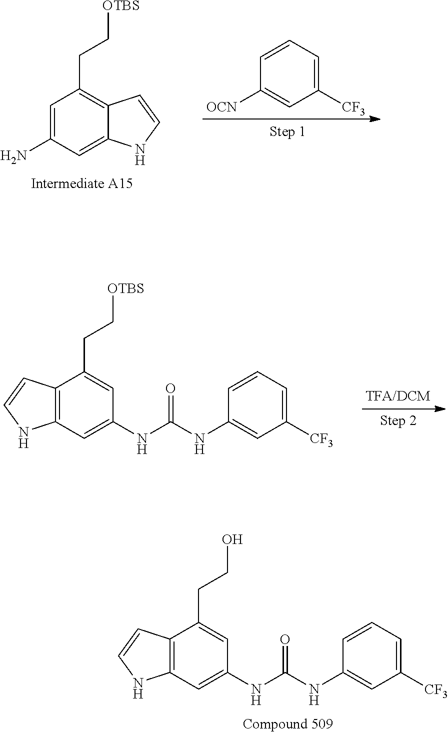

### Step 1: 1-(4-(2-((tert-butyldimethylsilyl)oxy)ethyl)-1H-indol-6-yl)-3-(3-(trifluoromethyl)phenyl)urea

4-[2-[(tert-butyldimethylsilyl)oxy]ethyl]-1H-indol-6-amine (110.0 mg, 0.4 mmol, 1.0 equiv.) was dissolved THF (10.0 mL), TEA (0.1 mL, 0.8 mmol, 2.0 equiv.) and 1-isocyanato-3-(trifluoromethyl)benzene (85.0 mg, 0.5 mmol, 1.2 equiv.) were added. The resulting solution was stirred for 1 hour at room temperature and then concentrated under vacuum. The residue was purified by flash column chromatography on silica gel, eluting with acetate/petroleum ether (1:1) to give 3-(4-[2-[(tert-butyldimethylsilyl)oxy]ethyl]-1H-indol-6-yl)-1-[3-(trifluoromethyl)phenyl]urea (100 mg, 55.3%) as yellow oil. LCMS Method CC: [M+H]⁺=478.

### Step 2: 3-[4-(2-hydroxyethyl)-1H-indol-6-yl]-1-[3-(trifluoromethyl)phenyl]urea

3-(4-[2-[(tert-butyldimethylsilyl)oxy]ethyl]-1H-indol-6-yl)-1-[3-(trifluoromethyl)phenyl]urea (120.0 mg, 0.3 mmol, 1.0 equiv.) was dissolved in DCM/TFA (5.0 mL/0.5 mL). The resulting solution was stirred for 1 hour at room temperature and then concentrated under vacuum. The crude product was purified by Prep-HPLC with the following conditions: Column, XSelect CSH Prep C18 OBD Column, Sum, 19*150 mm; mobile phase, Water (10 MMOL/L NH₄HCO₃) and ACN (42% Phase B up to 65% in 7 min); Detector, UV210/254 nm. This resulted in 3-[4-(2-hydroxyethyl)-1H-indol-6-yl]-1-[3-(trifluoromethyl)phenyl]urea (46.3 mg, 50.7%) as a white solid. LCMS Method CE: [M+H]⁺=364. ¹H NMR (400 MHz, DMSO-d₆) δ 10.91 (s, 1H), 9.61 (s, 1H), 9.09 (s, 1H), 8.04 (s, 1H), 7.65 (s, 1H), 7.60-7.58 (m, 1H), 7.52-7.48 (m, 1H), 7.27 (d, 1H), 7.21-7.19 (m, 1H), 6.74 (s, 1H), 6.40-6.38 (m, 1H), 4.68 (t, 1H), 3.71-3.66 (m, 2H), 2.94 (t, 2H).

The analogs prepared in the following table were prepared using the same method described for Example 185.

| Compound | Starting materials Used | Structure | LCMS data |
|---|---|---|---|
| Example 186 (Compound 501) | Intermediate A15; 1-fluoro-3-isocyanato-5- (trifluoromethyl)benzene | 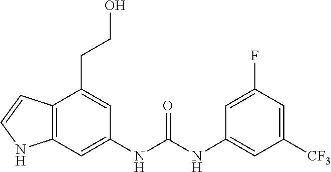 [SMILES: O=C(Nc1cc(F)cc(C(F)(F)F)c1)Nc1cc(CCO)c2cc[nH]c2c1] | Method CH: MS-ESI: 382 [M + H]⁺. |

### Example 187-188: 1-(3-(methylsulfonyl)-1H-indol-6-yl)-3-(4-(trifluoromethyl)phenyl)urea (Compound 616) and 1-(2-(methylsulfonyl)-1H-indol-6-yl)-3-(4-(trifluoromethyl)phenyl)urea (Compound 617)

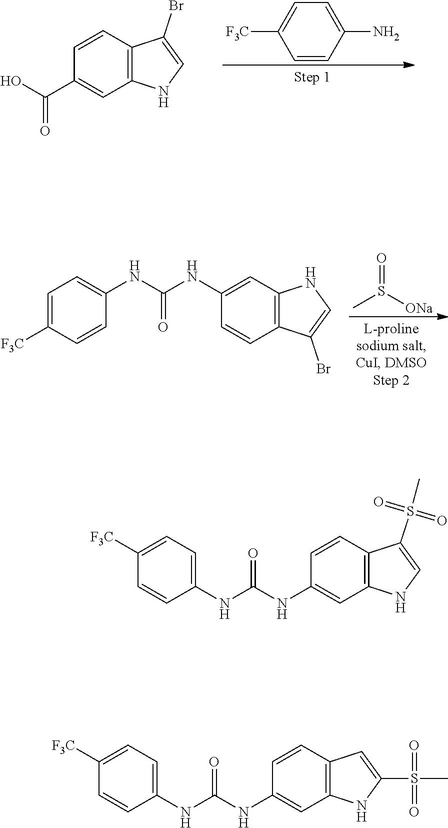

### Step 1: 1-(3-bromo-1H-indol-6-yl)-3-(4-(trifluoromethyl)phenyl)urea

3-Bromo-1H-indole-6-carboxylic acid (1.0 g, 4.2 mmol, 1.0 equiv.) was dissolved in toluene (10.0 mL) was dissolved in THF (50.0 mL), TEA (1.1 mL, 8.3 mmol, 2.0 equiv.) and DPPA (1.7 g, 6.3 mmol, 1.5 equiv.) were added. The reaction mixture was stirred for 2 hours at room temperature, then 4-(trifluoromethyl)aniline (0.7 g, 4.6 mmol, 1.1 equiv.) was added. The reaction mixture was stirred for additional 16 hours at 80° C. and quenched by the addition of water. The solution was extracted with ethyl acetate, dried over anhydrous sodium sulfate and concentrated under vacuum. The residue was purified by flash column chromatography on silica gel, eluting with acetate/petroleum ether (1:1) to give 1-(3-bromo-1H-indol-6-yl)-3-(4-(trifluoromethyl)phenyl)urea (630 mg) as a yellow solid. LCMS Method CH: [M+H]⁺=398.

### Step 2: 1-(3-(methylsulfonyl)-1H-indol-6-yl)-3-(4-(trifluoromethyl)phenyl)urea and 1-(2-(methylsulfonyl)-1H-indol-6-yl)-3-(4-(trifluoromethyl)phenyl)urea

1-(3-Bromo-1H-indol-6-yl)-3-(4-(trifluoromethyl)phenyl)urea (200.0 mg, 0.5 mmol, 1.0 equiv.) was dissolved in DMSO (5 mL), sodium methanesulfinate (77.0 mg, 0.7 mmol, 1.5 equiv.), L-proline sodium salt (14.0 mg, 0.1 mmol, 0.2 equiv.) and CuI (10.0 mg, 0.05 mmol, 0.1 equiv.) were added under nitrogen. The reaction mixture was stirred for 6 hours under 100° C. and then quenched by the addition of water. The solution was extracted with ethyl acetate, dried over anhydrous sodium sulfate and concentrated under vacuum. The residue was purified by flash column chromatography on silica gel, eluting with DCM/MeOH (10:1) to give crude product, which was further purified by Prep-HPLC with the following conditions: Column: XBridge Shield RP18 OBD Column, 30*150 mm, Sum; Mobile Phase A: Water (10 MMOL/L NH₄HCO₃+0.1% NH₃.H₂O), Mobile Phase B: ACN; Flow rate: 60 mL/min; Gradient: 38 B to 62 B in 7 min; 210/254 nm. This resulted in 1-(3-(methylsulfonyl)-1H-indol-6-yl)-3-(4-(trifluoromethyl)phenyl)urea (7.4 mg, 3.7%) as a light yellow solid and 1-(2-(methylsulfonyl)-1H-indol-6-yl)-3-(4-(trifluoromethyl)phenyl)urea (13.4 mg, 6.7%) as a light yellow solid.

Compound 616: LCMS Method CK: [M−H]⁻=396. ¹H NMR (400 MHz, DMSO-d₆): δ 12.02 (s, 1H), 9.07 (s, 1H), 8.92 (s, 1H), 7.97-7.92 (m, 2H), 7.69-7.62 (m, 5H), 7.12-7.10 (m, 1H), 3.17 (s, 3H).

Compound 617: LCMS Method CK: [M−H]⁻=396. ¹H NMR (400 MHz, DMSO-d₆): δ 12.12 (s, 1H), 9.10 (s, 1H), 8.96 (s, 1H), 7.92 (s, 1H), 7.67-7.60 (m, 5H), 7.06-7.03 (m, 2H), 3.30 (s, 3H).

### Example 189: 1-(4-azidophenyl)-3-(1H-indol-6-yl)urea (Compound 510)

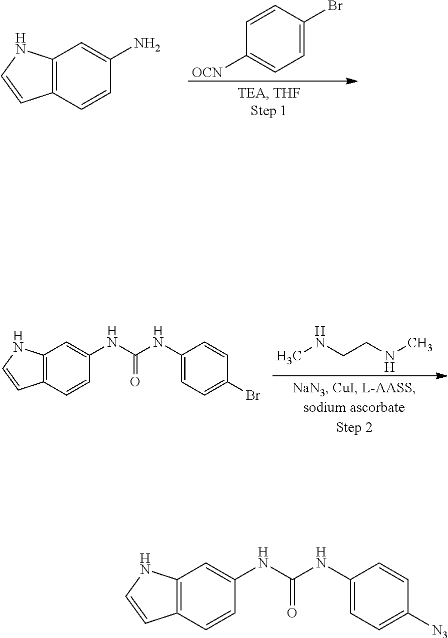

### Step 1: 1-(4-bromophenyl)-3-(1H-indol-6-yl)urea

1H-indol-6-amine (1.0 g, 7.6 mmol, 1.0 equiv.) was dissolved in THF (30 mL), 1-bromo-4-isocyanatobenzene (1.5 g, 7.6 mmol, 1.0 equiv.) and TEA (2.1 mL, 15.1 mmol, 2.0 equiv.) were added. The resulting mixture was stirred for 0.5 hour at room temperature and concentrated under vacuum. The residue was purified by flash column chromatography on silica gel, eluting with DCM/MeOH (10:1) to give 1-(4-bromophenyl)-3-(1H-indol-6-yl)urea (1.1 g, 44.0%) as a light brown solid. LCMS Method CC: [M+H]⁺=330.

### Step 2: 1-(4-azidophenyl)-3-(1H-indol-6-yl)urea

1-(4-Bromophenyl)-3-(1H-indol-6-yl)urea (300.0 mg, 0.9 mmol, 1.0 equiv.) was dissolved DMSO/water (10.0 mL/2.0 mL), NaN₃ (118.1 mg, 1.8 mmol, 2.0 equiv.), CuI (173.0 mg, 0.9 mmol, 1.0 equiv.), methyl[2-(methylamino)ethyl]amine (160.2 mg, 1.8 mmol, 2.0 equiv.) and sodium ascorbate (361.8 mg, 1.8 mmol, 2.0 equiv.) was added under nitrogen. The solution was stirred for overnight at 80° C. and then concentrated under vacuum. The residue was purified by flash column chromatography on silica gel, eluting with ethyl acetate/petroleum ether (1:1) to give crude product, which was further purified by Prep-HPLC with the following conditions: Column: XBridge Prep OBD C18 Column, 30×150 mm 5 um; Mobile Phase A: Water (10 MMOL/L NH₄HCO₃), Mobile Phase B: ACN; Flow rate: 60 mL/min; Gradient: 35 B to 65 B in 10 min; 254 nm. This resulted in 1-(4-azidophenyl)-3-(1H-indol-6-yl)urea (47.8 mg, 20.0%) as a off-white solid. LCMS Method CE: [M+H]⁺=293. ¹H NMR (300 MHz, DMSO-d₆): δ 10.91 (s, 1H), 8.67 (s, 1H), 8.52 (s, 1H), 7.77 (s, 1H), 7.51 (d, 2H), 7.40 (d, 1H), 7.22-7.20 (m, 1H), 7.04 (d, 2H), 6.86-6.83 (m, 1H), 6.32 (s, 1H).

### Example 190-191: (R)-1-(4-chloro-1H-indol-6-yl)-3-(1-(3-chloro-5-(trifluoromethyl)phenyl)ethyl)urea (Front Peak, Stereochem Unconfirmed) (Compound 521) and (S)-1-(4-chloro-1H-indol-6-yl)-3-(1-(3-chloro-5-(trifluoromethyl)phenyl)ethyl)urea (Second Peak, Stereochem Unconfirmed) (Compound 520)

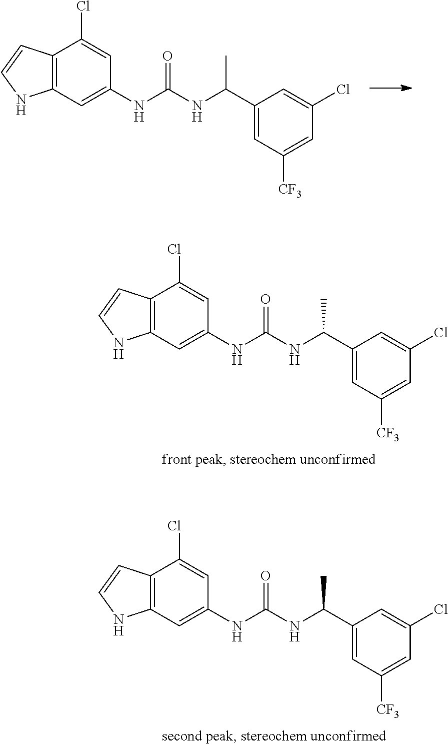 [SMILES: CC(NC(=O)Nc1cc(Cl)c2cc[nH]c2c1)c1cc(Cl)cc(C(F)(F)F)c1 ; C[C@@H](NC(=O)Nc1cc(Cl)c2cc[nH]c2c1)c1cc(Cl)cc(C(F)(F)F)c1 ; C[C@H](NC(=O)Nc1cc(Cl)c2cc[nH]c2c1)c1cc(Cl)cc(C(F)(F)F)c1]

The racemic 1-(4-chloro-1H-indol-6-yl)-3-(1-(3-chloro-5-(trifluoromethyl)phenyl)ethyl)urea (250.0 mg) was resolved by Prep chiral HPLC with the following conditions: Column: CHIRALPAK IG, 2*25 cm, 5 um; Mobile Phase A: Hex:DCM=3:1(0.5% 2M NH₃-MeOH)—HPLC, Mobile Phase B: IPA—HPLC; Flow rate: 20 mL/min; Gradient: 20 B to 20 B in 7.5 min; 220/254 nm; RT1:3.242; RT2:5.168. This resulted in front peak (106.4 mg) as a off-white solid and second peak (123.2 mg) as a off-white solid.

Compound 521: LCMS Method CH: [M+H]⁺=416. ¹H NMR (300 MHz, DMSO-d₆): δ 11.19 (s, 1H), 8.52 (s, 1H), 7.78-7.73 (m, 3H), 7.55 (s, 1H), 7.31-7.29 (m, 1H), 7.07 (s, 1H), 6.81 (d, 1H), 6.35-6.32 (m, 1H), 4.97-4.91 (m, 1H), 1.44 (d, 3H).

Compound 520: LCMS Method CH: [M+H]⁺=416. ¹H NMR (300 MHz, DMSO-d₆): δ 11.18 (s, 1H), 8.52 (s, 1H), 7.78-7.73 (m, 3H), 7.55 (s, 1H), 7.31-7.29 (m, 1H), 7.07 (s, 1H), 6.81 (d, 1H), 6.35-6.32 (m, 1H), 4.95-4.88 (m, 1H), 1.44 (d, 3H).

### Example 192: 1-(1-acetyl-3-fluoro-1H-indol-6-yl)-3-(4-(trifluoromethyl)phenyl)urea (Compound 534)

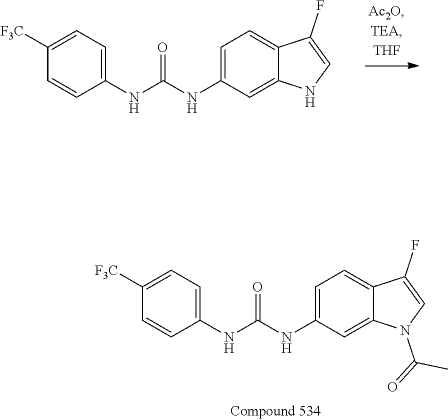 [SMILES: O=C(Nc1ccc(C(F)(F)F)cc1)Nc1ccc2c(F)c[nH]c2c1 ; CC(=O)n1cc(F)c2ccc(NC(=O)Nc3ccc(C(F)(F)F)cc3)cc21]

3-(3-fluoro-1H-indol-6-yl)-1-[4-(trifluoromethyl)phenyl]urea (100.0 mg, 0.3 mmol, 1.0 equiv.) was dissolved in THF (20.0 mL), acetic anhydride (302.7 mg, 3.0 mmol, 10.0 equiv.) and TEA (0.1 mL, 0.9 mmol, 3.0 equiv.) were added. The resulting mixture was stirred for 2 hours at room temperature and then quenched by the addition of water. The aqueous layer was extracted with ethyl acetate, washed with brine, dried over anhydrous Na₂SO₄ and concentrated under reduced pressure. The crude product was purified by Prep-HPLC with the following conditions: Column: XBridge Shield RP18 OBD Column, 5 um, 19*150 mm; Mobile Phase A: Water (10 MMOL/L NH₄HCO₃+0.1% NH₃.H₂O), Mobile Phase B: ACN; Flow rate: 60 mL/min; Gradient: 50 B to 70 B in 7 min; 210/254 nm. This resulted in 1-(1-acetyl-3-fluoro-1H-indol-6-yl)-3-(4-(trifluoromethyl)phenyl)urea (17.1 mg, 14.3%) as a off-white solid. LCMS Method CI: [M+H]⁺=380. ¹H NMR (400 MHz, DMSO-d₆): δ 9.13 (s, 1H), 9.07 (s, 1H), 8.66 (s, 1H), 7.84 (s, 1H), 7.68-63 (m, 4H), 7.56-7.54 (m, 1H), 7.49-7.47 (m, 1H), 2.58 (s, 3H).

### Example 193: 7-fluoro-N-(1H-indol-6-yl)quinolin-2-amine (Compound 559)

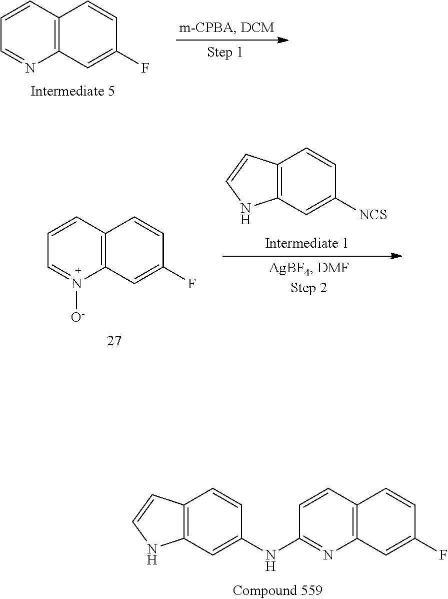

### Step 1: 7-fluoroquinoline 1-oxide

7-fluoroquinoline was dissolved (580.0 mg, 3.9 mmol, 1.0 equiv.) in DCM (10.0 mL), then m-CPBA (1.3 g, 5.9 mmol, 1.5 equiv., 75%) was added. The resulting solution was stirred for 4 hours at ambient temperature and concentrated under vacuum. The residue was purified by flash column chromatography on silica gel, eluting with dichloromethane/methanol (20:1) to give 7-fluoroquinoline 1-oxide (550 mg) as a white solid. LCMS Method CA: [M+H]⁺=164.

### Step 2: 7-fluoro-N-(1H-indol-6-yl)quinolin-2-amine

7-fluoroquinoline 1-oxide (200.0 mg, 1.2 mmol, 1.0 equiv.) was dissolved in DMF (5.0 mL), then AgBF₄ (477.2 mg, 2.4 mmol, 2.0 equiv.) and 6-isothiocyanato-1H-indole (256.2 mg, 1.4 mmol, 1.2 equiv.) were added. The resulting solution was stirred for 4 hours at ambient temperature and concentrated under vacuum. The crude product was purified by Prep-HPLC with the following conditions: Column: XBridge Prep OBD Cat Column, 30*150 mm, 5 um; mobile phase: Water (2 mmol/L NH₄HCO₃) and ACN (20 Phase B up to 80% in 8 min); Detector, UV 220/254 nm, RTL: 7.82 min 7-fluoro-N-(1H-indol-6-yl)quinolin-2-amine (73.1 mg, 21.3%) was obtained as a white solid. LCMS Method CD: [M+H]+=278. ¹H NMR (400 MHz, DMSO-d₆) δ 8.46-8.45 (m, 1H), 8.02 (d, 1H), 7.79-7.76 (m, 1H), 7.46 (d, 1H), 7.30 (dd, 1H), 7.25-7.20 (m, 2H), 7.15 (t, 1H), 7.01 (d, 1H), 6.36 (d, 1H).

The analogs prepared in the following table were prepared using the same method described for Example 193.

| Compound | Starting materials Used | Structure | LCMS data |
|---|---|---|---|
| Example 194 (Compound 556) | Intermediate B1; Intermediate B6 | 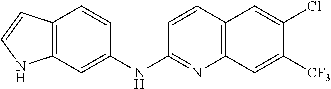 [SMILES: FC(F)(F)c1cc2nc(Nc3ccc4cc[nH]c4c3)ccc2cc1Cl] | Method CI: MS-ESI: 362 [M + H]⁺. |
| Example 195 (Compound 553) | Intermediate B1; Intermediate B7 | 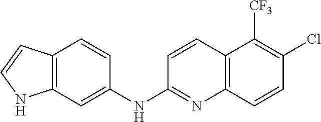 [SMILES: FC(F)(F)c1c(Cl)ccc2nc(Nc3ccc4cc[nH]c4c3)ccc12] | Method CI: MS-ESI: 362 [M + H]⁺. |
| Example 196 (Compound 554) | Intermediate B1; Intermediate B8 | 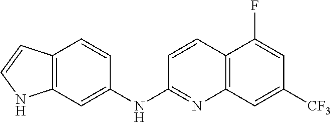 [SMILES: Fc1cc(C(F)(F)F)cc2nc(Nc3ccc4cc[nH]c4c3)ccc12] | Method CE: MS-ESI: 346 [M + H]⁺. |
| Example 197 (Compound 558) | Intermediate B1; Intermediate B9 | 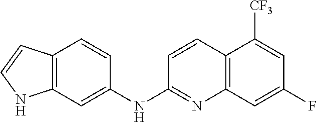 [SMILES: Fc1cc(C(F)(F)F)c2ccc(Nc3ccc4cc[nH]c4c3)nc2c1] | Method CE: MS-ESI: 346 [M + H]⁺. |
| Example 198 (Compound 564) | Intermediate B1; Intermediate B10 | 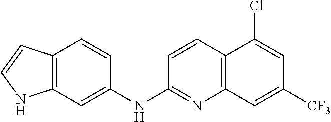 [SMILES: FC(F)(F)c1cc(Cl)c2ccc(Nc3ccc4cc[nH]c4c3)nc2c1] | Method CH: MS-ESI: 362 [M + H]⁺. |
| Example 199 (Compound 563) | Intermediate B1; Intermediate B11 | 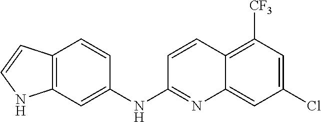 [SMILES: FC(F)(F)c1cc(Cl)cc2nc(Nc3ccc4cc[nH]c4c3)ccc12] | Method CH: MS-ESI: 362 [M + H]⁺. |
| Example 200 (Compound 565) | Intermediate B1; Intermediate B12 | 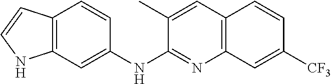 [SMILES: Cc1cc2ccc(C(F)(F)F)cc2nc1Nc1ccc2cc[nH]c2c1] | Method CI: MS-ESI: 342 [M + H]⁺. |
| Example 201 (Compound 589) | Intermediate B1; 7-chloroquinoline | 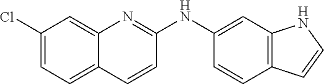 [SMILES: Clc1ccc2ccc(Nc3ccc4cc[nH]c4c3)nc2c1] | Method CE: MS-ESI: 294 [M + H]⁺. |
| Example 202 (Compound 592) | Intermediate B1; 7-methylquinoline | 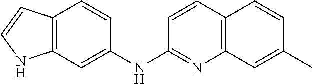 [SMILES: Cc1ccc2ccc(Nc3ccc4cc[nH]c4c3)nc2c1] | Method CE: MS-ESI: 274 [M + H]⁺. |
| Example 203 (Compound 607) | Intermediate B1; 7-bromoquinoline | 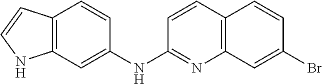 [SMILES: Brc1ccc2ccc(Nc3ccc4cc[nH]c4c3)nc2c1] | Method CE: MS-ESI: 338 [M + H]⁺. |
| Example 204 (Compound 381) | Intermediate B1; 7-(trifluoromethyl)quinoline | 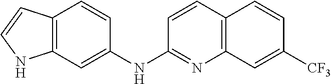 [SMILES: FC(F)(F)c1ccc2ccc(Nc3ccc4cc[nH]c4c3)nc2c1] | Method CI: MS-ESI: 328 [M + H]⁺. |
| Example 205 (Compound 557) | Intermediate B2; 7-(trifluoromethyl)quinoline | 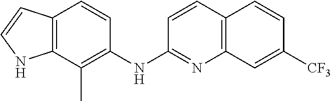 [SMILES: Cc1c(Nc2ccc3ccc(C(F)(F)F)cc3n2)ccc2cc[nH]c12] | Method CE: MS-ESI: 342 [M + H]⁺. |
| Example 206 (Compound 604) | Intermediate B3; 7-(trifluoromethyl)quinoline | 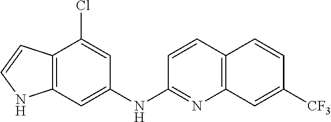 [SMILES: FC(F)(F)c1ccc2ccc(Nc3cc(Cl)c4cc[nH]c4c3)nc2c1] | Method CI: MS-ESI: 362 [M + H]⁺. |
| Example 207 (Compound 387) | Intermediate B4; 7-chloroquinoline | 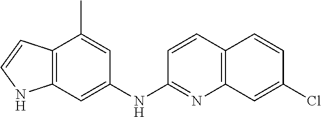 [SMILES: Cc1cc(Nc2ccc3ccc(Cl)cc3n2)cc2[nH]ccc12] | Method CE: MS-ESI: 308 [M + H]⁺. |

### Example 208: N-(1H-indol-6-yl)-5,6,7,8-tetrahydroquinolin-2-amine (Compound 567)

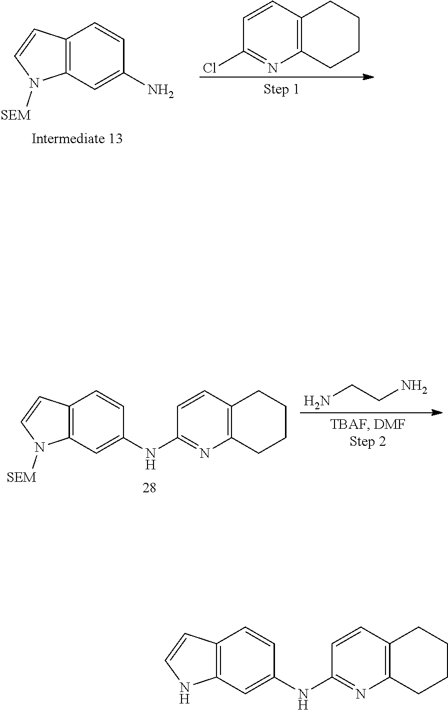

### Step 1: N-(1-[[2-(trimethylsilyl)ethoxy]methyl]indol-6-yl)-5,6,7,8-tetrahydroquinolin-2-amine

2-chloro-5,6,7,8-tetrahydroquinoline (250.0 mg, 1.5 mmol, 1.0 equiv.) was dissolved in dioxane (5.0 mL), 1-[[2-(trimethylsilyl)ethoxy]methyl]indol-6-amine (391.4 mg, 1.5 mmol, 1.0 equiv.), Pd₂(dba)₃(136.6 mg, 0.1 mmol, 0.1 equiv.), XPhos (71.1 mg, 0.1 mmol, 0.1 equiv.), Cs₂CO₃ (971.8 mg, 2.9 mmol, 2.0 equiv.) were added under the atmosphere of N₂. The resulting mixture was stirred for 16 h at 90° C. and then quenched by the addition of water. The resulting mixture was extracted with ethyl acetate, dried over anhydrous Na₂SO₄ and concentrated under reduced pressure. The residue was purified by flash column chromatography on silica gel, eluting with ethyl acetate/petroleum ether (1:2) to give N-(1-[[2-(trimethylsilyl)ethoxy]methyl]indol-6-yl)-5,6,7,8-tetrahydroquinolin-2-amine (200.0 mg, 34.1%) as a brown solid. LCMS Method CA: [M−H]⁻=392.

### Step 2: N-(1H-indol-6-yl)-5,6,7,8-tetrahydroquinolin-2-amine

N-(1-[[2-(trimethylsilyl)ethoxy]methyl]indol-6-yl)-5,6,7,8-tetrahydroquinolin-2-amine (190.0 mg, 0.5 mmol, 1.0 equiv.) was dissolved in DMF (10.0 mL), ethylenediamine (58.0 mg, 0.9 mmol, 2.0 equiv.), TBAF (252.4 mg, 0.9 mmol, 2.0 equiv.) were added. The resulting mixture was stirred for 2 hours at 80° C. under nitrogen atmosphere and then quenched by the addition of water. The resolution solution was extracted with ethyl acetate, dried over anhydrous Na₂SO₄ and concentrated under reduced pressure. The residue was purified by flash column chromatography on silica gel, eluting with ethyl acetate/petroleum ether (1:1) to give the crude product, which was further purified by Pre-HPLC with the following conditions: Column: XBridge Prep OBD C18 Column, 30×150 mm Sum; Mobile Phase A: Water (0.05% NH₃H₂O ), Mobile Phase B: ACN; Flow rate: 60 mL/min; Gradient: 45 B to 60 B in 7 min; 254 nm; RTL: 6.0 min. N-(1H-indol-6-yl)-5,6,7,8-tetrahydroquinolin-2-amine (46.6 mg, 36.3%) was obtained as a yellow solid. LCMS Method CE: [M+H]⁺=264. ¹H NMR (400 MHz, DMSO-d₆) δ 10.82 (s, 1H), 8.57 (s, 1H), 7.97 (s, 1H), 7.37 (d, 1H), 7.21-7.15 (m, 2H), 7.02-7.00 (m, 1H), 6.61 (d, 1H), 6.30 (s, 1H), 2.74-2.71 (m, 2H), 2.61-2.58 (m, 2H), 1.84-1.74 (m, 4H).

The analogs prepared in the following table were prepared using the same method described for Example 208.

| Compound | Starting materials Used | Structure | LCMS data |
|---|---|---|---|
| Example 209 (Compound 578) | Intermediate B13; 2-chloro-4-methyl-6- (trifluoromethyl)pyridine | 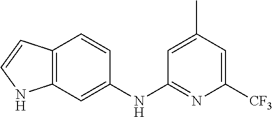 [SMILES: Cc1cc(Nc2ccc3cc[nH]c3c2)nc(C(F)(F)F)c1] | Method CE: MS- ESI: 292 [M + H]+. |
| Example 210 (Compound 579) | Intermediate B13; 7-bromo-2- (trifluoromethyl)quinoline | 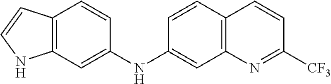 [SMILES: FC(F)(F)c1ccc2ccc(Nc3ccc4cc[nH]c4c3)cc2n1] | Method CE: MS- ESI: 328 [M + H]+. |
| Example 211 (Compound 591) | Intermediate B13; 2,6-dichloro-3- (trifluoromethyl)pyridine | 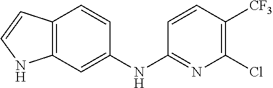 [SMILES: FC(F)(F)c1ccc(Nc2ccc3cc[nH]c3c2)nc1Cl] | Method CE: MS- ESI: 312 [M + H]+. |
| Example 212 (Compound 609) | Intermediate B13; 2-chloro-6- (trifluoromethyl)pyridine | 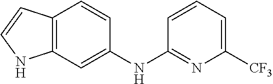 [SMILES: FC(F)(F)c1cccc(Nc2ccc3cc[nH]c3c2)n1] | Method CE: MS- ESI: 278 [M + H]+. |
| Example 213 (Compound 385) | Intermediate B13; Intermediate B18 | 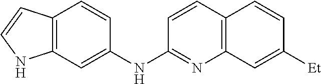 [SMILES: CCc1ccc2ccc(Nc3ccc4cc[nH]c4c3)nc2c1] | Method CI: MS- ESI: 288 [M + H]+. |
| Example 214 (Compound 385) | Intermediate B13; Intermediate B19 | 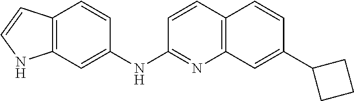 [SMILES: c1cc2ccc(Nc3ccc4ccc(C5CCC5)cc4n3)cc2[nH]1] | Method CI: MS- ESI: 314 [M + H]+. |
| Example 215 (Compound 621) | Intermediate B13; 2-bromo-5-cyclobutylpyridine | 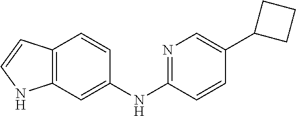 [SMILES: c1cc2ccc(Nc3ccc(C4CCC4)cn3)cc2[nH]1] | Method CI: MS- ESI: 264 [M + H]+. |
| Example 216 (Compound 622) | Intermediate B13; Intermediate B17 | 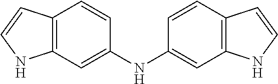 [SMILES: c1cc2ccc(Nc3ccc4cc[nH]c4c3)cc2[nH]1] | Method CI: MS- ESI: 248 [M + H]+. |
| Example 217 (Compound 421) | Intermediate B13; 6-chloro-3-methyl-2- (trifluoromethyl)pyridine | 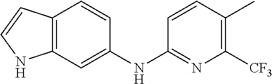 [SMILES: Cc1ccc(Nc2ccc3cc[nH]c3c2)nc1C(F)(F)F] | Method CE: MS- ESI: 292 [M + H]+. |
| Example 218 (Compound 401) | Intermediate B13; 2-chloro-4-phenylpyridine | 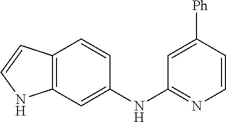 [SMILES: c1ccc(-c2ccnc(Nc3ccc4cc[nH]c4c3)c2)cc1] | Method CE: MS- ESI: 286 [M + H]+. |
| Example 219 (Compound 390) | Intermediate B13; 2-chloro-7- (trifluoromethyl)quinazoline | 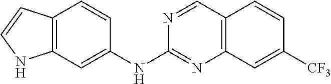 [SMILES: FC(F)(F)c1ccc2cnc(Nc3ccc4cc[nH]c4c3)nc2c1] | Method CI: MS- ESI: 329 [M + H]+. |
| Example 220 (Compound 623) | Intermediate B13; 2-chloro-6- (trifluoromethyl)quinoline | 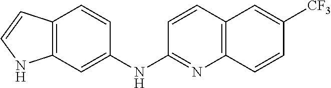 [SMILES: FC(F)(F)c1ccc2nc(Nc3ccc4cc[nH]c4c3)ccc2c1] | Method CI: MS- ESI: 328 [M + H]+. |
| Example 221 (Compound 624) | Intermediate B13; Intermediate B22 | 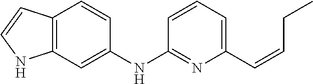 [SMILES: CC/C=C\c1cccc(Nc2ccc3cc[nH]c3c2)n1] | Method CE: MS- ESI: 264 [M + H]+. |
| Example 222 (Compound 384) | Intermediate B14; 2-chloro-7- (trifluoromethyl)quinoline | 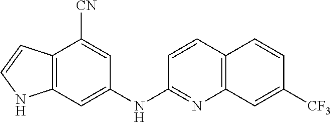 [SMILES: N#Cc1cc(Nc2ccc3ccc(C(F)(F)F)cc3n2)cc2[nH]ccc12] | Method CI: MS- ESI: 353 [M + H]+. |
| Example 223 (Compound 380) | Intermediate B15; 2-chloro-7- (trifluoromethyl)quinoline | 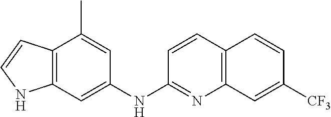 [SMILES: Cc1cc(Nc2ccc3ccc(C(F)(F)F)cc3n2)cc2[nH]ccc12] | Method CI: MS- ESI: 342 [M + H]+. |
| Example 224 (Compound 383) | Intermediate B16; 2-chloro-7- (trifluoromethyl)quinoline | 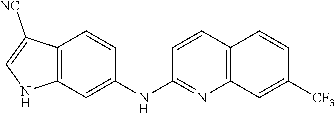 [SMILES: N#Cc1c[nH]c2cc(Nc3ccc4ccc(C(F)(F)F)cc4n3)ccc12] | Method CI: MS- ESI: 353 [M + H]+. |
| Example 225 (Compound 625) | Intermediate B15; 2-bromo-6-phenylpyridine | 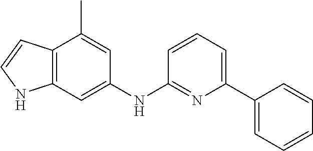 [SMILES: Cc1cc(Nc2cccc(-c3ccccc3)n2)cc2[nH]ccc12] | Method CI: MS- ESI: 300 [M + H]+. |
| Example 226 (Compound 424) | 5-phenylpyridin-2-amine; Intermediate B17 | 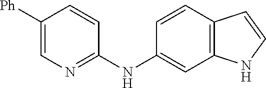 [SMILES: c1ccc(-c2ccc(Nc3ccc4cc[nH]c4c3)nc2)cc1] | Method CE: MS- ESI: 286 [M + H]+. |
| Example 227 (Compound 427) | 6-phenylpyridin-2-amine; Intermediate B17 | 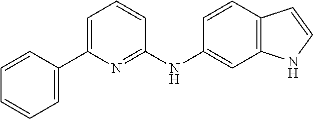 [SMILES: c1ccc(-c2cccc(Nc3ccc4cc[nH]c4c3)n2)cc1] | Method CE: MS- ESI: 286 [M + H]+. |
| Example 228 (Compound 626) | 4-phenylpyrimidin-2-amine; Intermediate B17 | 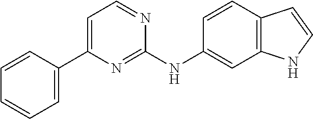 [SMILES: c1ccc(-c2ccnc(Nc3ccc4cc[nH]c4c3)n2)cc1] | Method CE: MS- ESI: 287 [M + H]+. |

### Example 229: N-(1-methyl-1H-indol-6-yl)-7-(trifluoromethyl)quinolin-2-amine (Compound 608)

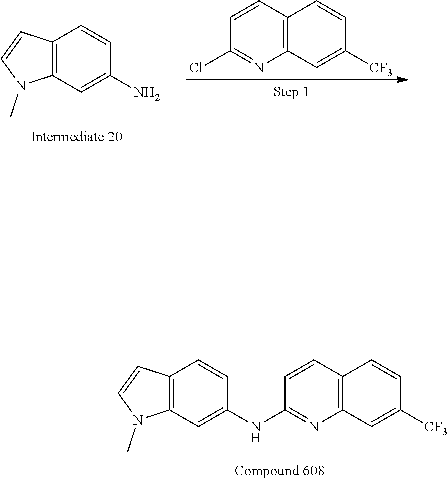 [SMILES: Cn1ccc2ccc(N)cc21 ; FC(F)(F)c1ccc2ccc(Cl)nc2c1 ; Cn1ccc2ccc(Nc3ccc4ccc(C(F)(F)F)cc4n3)cc21]

1-methylindol-6-amine (200.00 mg, 1.4 mmol, 1.0 equiv.), was dissolved in dioxane (6.0 mL), 2-chloro-7-(trifluoromethyl)quinoline (316.8 mg, 1.4 mmol, 1.0 equiv.), Cs₂CO₃ (891.5 mg, 2.7 mmol, 2.0 equiv.), Xphos (65.2 mg, 0.1 mmol, 0.1 equiv.), Pd₂(dba)₃ (125.3 mg, 0.1 mmol, 0.1 equiv.) were added under atmosphere of nitrogen. The resulting solution was stirred for 4 hours at 90° C. and concentrated under vacuum. The residue was purified by flash column chromatography on silica gel, eluting with ethyl acetate/petroleum ether (1:3) to give the crude product, which was further purified by Prep-HPLC with the following conditions: Column, XBridge Shield RP18 OBD Column, 5 um, 19*150 mm; mobile phase, Water (10 MMOL/L NH₄HCO₃+0.1% NH₃.H₂O) and ACN (57% Phase B up to 77% in 7 min); Detector, uv 254 nm. N-(1-Methylindol-6-yl)-7-(trifluoromethyl)quinolin-2-amine (67.6 mg, 14.3%) was isolated as a light yellow solid. LCMS Method CI: [M+H]⁺=342.

¹H NMR (400 MHz, DMSO-d₆) δ 9.63 (s, 1H), 8.47 (s, 1H), 8.14 (d, 1H), 7.98-7.93 (m, 2H), 7.53-7.49 (m, 2H), 7.31-7.21 (m, 3H), 6.38 (s, 1H), 3.83 (s, 3H).

The analogs prepared in the following table were prepared using the same method described for Example 229.

| Compound | Starting materials Used | Structure | LCMS data |
|---|---|---|---|
| Example 230 (Compound 568) | 5-chloro-6- (trifluoromethyl)pyridin-2-amine 6-bromo-4-chloro-1H-indole | 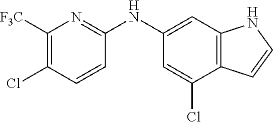 [SMILES: FC(F)(F)c1nc(Nc2cc(Cl)c3cc[nH]c3c2)ccc1Cl] | Method CE: MS-ESI: 346 [M + H]⁺. |
| Example 231 (Compound 566) | 1H-pyrrolo[2,3-b]pyridin-6-amine 2-chloro-6,7-dihydro-5H- cyclopenta[b]pyridine | 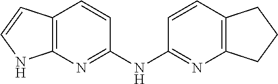 [SMILES: c1cc2ccc(Nc3ccc4c(n3)CCC4)nc2[nH]1] | Method CE: MS-ESI: 251 [M + H]⁺. |
| Example 232 (Compound 388) | Intermediate B21 6-bromo-1H-indole | 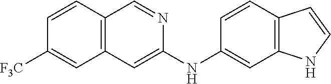 [SMILES: FC(F)(F)c1ccc2cnc(Nc3ccc4cc[nH]c4c3)cc2c1] | Method CC: MS-ESI: 328 [M + H]⁺. |
| Example 233 (Compound 653) | 5-(trifluoromethyl)-1H- benzo[d]imidazol-2-amine 6-bromo-1H-indole | 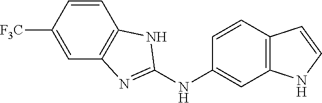 [SMILES: FC(F)(F)c1ccc2[nH]c(Nc3ccc4cc[nH]c4c3)nc2c1] | Method CE: MS-ESI: 317 [M + H]⁺. |
| Example 234 (Compound 654) | 5-(trifluoro- methyl)benzo[d\|thiazol-2-amine 6-bromo-1H-indole | 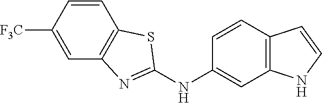 [SMILES: FC(F)(F)c1ccc2sc(Nc3ccc4cc[nH]c4c3)nc2c1] | Method CE: MS-ESI: 334 [M + H]+. |

### Example 235: 2-((1H-indol-6-yl)amino)quinazolin-4(3H)-one (Compound 655)

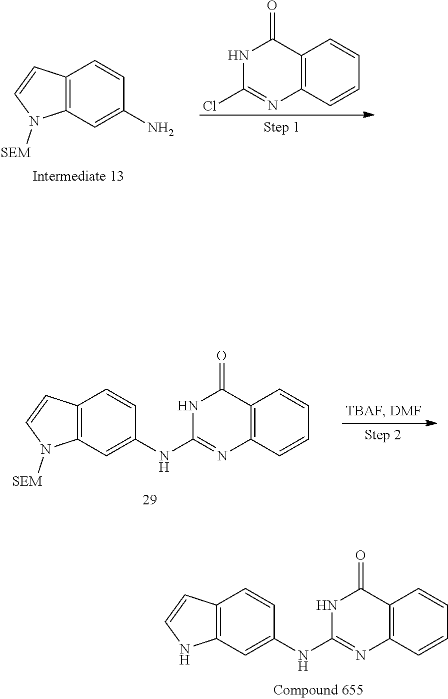 [SMILES: C[Si](C)(C)CCOCn1ccc2ccc(N)cc21 ; O=c1[nH]c(Cl)nc2ccccc12 ; C[Si](C)(C)CCOCn1ccc2ccc(Nc3nc4ccccc4c(=O)[nH]3)cc21 ; O=c1[nH]c(Nc2ccc3cc[nH]c3c2)nc2ccccc12]

### Step 1: 2-((1-((2-(trimethylsilyl)ethoxy)methyl)-1H-indol-6-yl)amino)quinazolin-4(3H)-one

1-[[2-(trimethylsilyl)ethoxy]methyl]indol-6-amine (200.0 mg, 0.8 mmol, 1.0 equiv.) was dissolved in DMSO (20.0 mL), then 2-chloro-3H-quinazolin-4-one (137.6 mg, 0.8 mmol, 1.0 equiv.), DIEA (985.0 mg, 7.6 mmol, 10 equiv.) were added. The resulting solution was stirred for 16 hours at 80° C. and then quenched by the addition of water. The resulting solution extracted with ethyl acetate, washed with brine, dried over anhydrous Na₂SO₄ and concentrated under reduced pressure. The residue was purified by flash column chromatography on silica gel, eluting with ethyl acetate/petroleum ether (1:2) to give 2-[(1-[[2-(trimethylsilyl)ethoxy]methyl]indol-6-yl)amino]-3H-quinazolin-4-one (233 mg, 75.2%) as a yellow solid. LCMS Method CA: [M+H]⁺=407.

### Step 2: 2-((1H-indol-6-yl)amino)quinazolin-4(3H)-one

2-[(1-[[2-(trimethylsilyl)ethoxy]methyl]indol-6-yl)amino]-3H-quinazolin-4-one (150.0 mg, 0.4 mmol, 1.0 equiv.) was dissolved in DMF (15.0 mL), ethylenediamine (44.4 mg, 0.7 mmol, 2.0 equiv.), TBAF (192.9 mg, 0.7 mmol, 2.0 equiv.) were added. The resulting mixture was stirred for 16 hours at 80° C. and then quenched by the addition of water. The resulting mixture was extracted with ethyl acetate, washed with brine, dried over anhydrous Na₂SO₄ and concentrated under reduced pressure. The residue was purified by flash column chromatography on silica gel, eluting with ethyl acetate/petroleum ether (1:1) to give the crude product, which was further purified by Prep-HPLC with the following conditions: Column: SunFire Prep C18 OBD Column, 19×150 mm 5 um 10 nm; Mobile Phase A: Water (0.05% FA), Mobile Phase B: ACN; Flow rate: 20 mL/min; Gradient: 12 B to 37 B in 11 min; 254 nm; RTL: 10. ). 2-((1H-indol-6-yl)amino)quinazolin-4(3H)-one (21.3 mg, 20.9%) was obtained as a white solid. LCMS Method CI: [M+H]⁺=277. ¹H NMR (300 MHz, DMSO-d₆) δ 11.04 (s, 1H), 10.75 (brs, 1H), 8.14-8.12 (m, 1H), 7.96 (d, 1H), 7.66 (t, 1H), 7.49 (d, 1H), 7.38-7.36 (m, 1H), 7.27 (d, 1H), 7.21 (t, 1H), 7.03-6.99 (m, 1H), 6.38 (d, 1H).

The analogs prepared in the following table were prepared using the same method described for Example 235.

| Compound | Starting materials Used | Structure | LCMS data |
|---|---|---|---|
| Example 236 (Compound 555) | Intermediate B13; 2-chloro-7- (trifluoromethyl)quinoxaline | 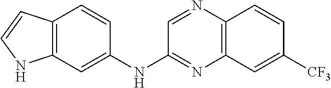 [SMILES: FC(F)(F)c1ccc2ncc(Nc3ccc4cc[nH]c4c3)nc2c1] | Method CH: MS-ESI: 329 [M + H]⁺. |

### Example 237: N-(5-methyl-1H-indol-6-yl)-7-(trifluoromethyl)quinolin-2-amine (Compound 580)

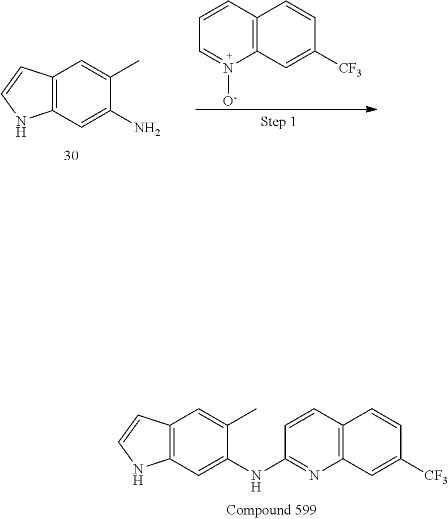

5-Methyl-1H-indol-6-amine (200.0 mg, 1.4 mmol, 1.0 equiv.) was dissolved in DCE (15.0 mL), then DIEA (353.6 mg, 2.7 mmol, 2.0 equiv.), 7-(trifluoromethyl)quinolin-1-ium-1-olate (291.6 mg, 1.4 mmol, 1.0 equiv.), PyBrOP (1.3 g, 2.7 mmol, 2.0 equiv.) were added. The resulting solution was stirred for 16 hours at 80° C. and then concentrated under vacuum. The residue was purified by flash column chromatography on silica gel, eluting with ethyl acetate/petroleum ether (1:4) to give the crude product, which was further purified by Prep-HPLC with the following conditions: Column, XBridge Shield RP18 OBD Column, 19*250 mm, 10 um; mobile phase, Water (10 MMOL/L NH₄HCO₃+0.1% NH₃.H₂O) and ACN (50% Phase B up to 80% in 7 min); Detector, UV 254 nm. N-(5-Methyl-1H-indol-6-yl)-7-(trifluoromethyl)quinolin-2-amine (46.7 mg, 10.0%) was obtained as a yellow solid. LCMS Method CE: [M+H]⁺=342. ¹H NMR (400 MHz, DMSO-d₆) δ 10.93 (s, 1H), 8.79 (s, 1H), 8.10 (d, 1H), 7.91 (d, 1H), 7.77 (s, 1H), 7.73 (s, 1H), 7.46-7.44 (m, 1H), 7.41 (s, 1H), 7.29-7.28 (m, 1H), 7.15-7.12 (m, 1H), 6.35-6.34 (m, 1H), 2.30 (s, 3H).

### Example 240: N-(6-butylpyridin-2-yl)-1H-indol-6-amine (Compound 628)

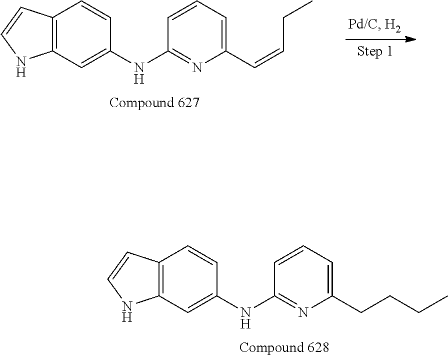 [SMILES: CC/C=C\c1cccc(Nc2ccc3cc[nH]c3c2)n1 ; CCCCc1cccc(Nc2ccc3cc[nH]c3c2)n1]

(Z)-N-(6-(but-1-en-1-yl)pyridin-2-yl)-1H-indol-6-amine (500.0 mg, 1.9 mmol, 1.0 equiv.) was dissolved in CH₃OH (10.0 mL), Pd/C (27.0 mg, 0.3 mmol, 0.2 equiv.) wad added under nitrogen. The mixture was degassed and back filled with hydrogen for three times, then stirred for 3 hours at room temperature under atmosphere of hydrogen. After filtration, the filtrate was concentrated under reduced pressure. The residue was purified by Prep-HPLC with the following conditions: Column: YMC-Actus Triart C18, 30*250, Sum; Mobile Phase A: Water (10 MMOL/L NH₄HCO₃+0.1% NH₃.H₂O), Mobile Phase B: ACN; Flow rate: 60 mL/min; Gradient: 50 B to 80 B in 7 min; 254/210 nm; RT1:6.9. N-(6-butylpyridin-2-yl)-1H-indol-6-amine (142 mg, 28.4%) was isolated as an off-white solid. LCMS Method CE: [M+H]⁺=266. ¹H NMR (300 MHz, DMSO-d₆) δ 11.86 (brs, 1H), 8.71 (s, 1H), 7.97 (s, 1H), 7.42-7.38 (m, 2H), 7.17 (t, 1H), 7.07-7.03 (m, 1H), 6.62 (d, 1H), 6.52 (d, 1H), 6.31 (s, 1H), 2.62 (t, 2H), 1.23-1.68 (m, 2H), 1.39-1.32 (m, 2H), 0.93 (t, 6H)

### Examples 241-242: 7-cyclohexyl-N-(1H-indol-6-yl)quinolin-2-amine (Compound 389) and 7-(cyclohex-1-en-1-yl)-N-(1H-indol-6-yl)quinolin-2-amine (Compound 606)

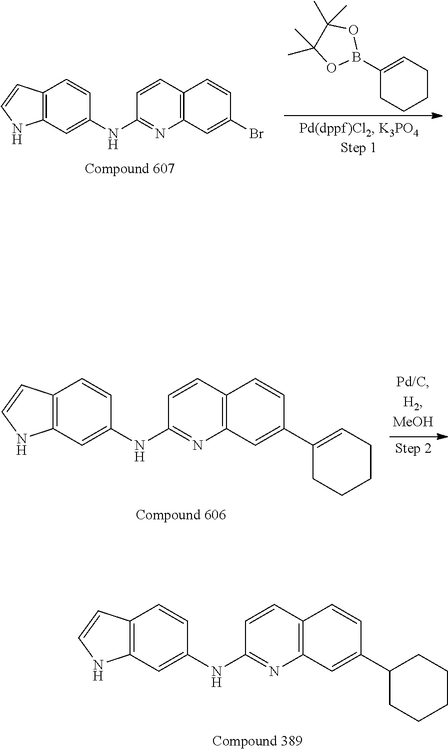 [SMILES: Brc1ccc2ccc(Nc3ccc4cc[nH]c4c3)nc2c1 ; CC1(C)OB(C2=CCCCC2)OC1(C)C ; C1=C(c2ccc3ccc(Nc4ccc5cc[nH]c5c4)nc3c2)CCCC1 ; c1cc2ccc(Nc3ccc4ccc(C5CCCCC5)cc4n3)cc2[nH]1]

### Step 1: 7-(cyclohex-1-en-1-yl)-N-(1H-indol-6-yl)quinolin-2-amine

7-Bromo-N-(1H-indol-6-yl)quinolin-2-amine (100.0 mg, 0.3 mmol, 1.0 equiv.), was dissolved in dioxane/water(5.0 mL/1.0 mL), then cyclohex-1-en-1-ylboronic acid (74.5 mg, 0.6 mmol, 2.0 equiv.), K₃PO₄ (125.5 mg, 0.6 mmol, 2.0 equiv.) and Pd(dppf)Cl₂ (21.6 mg, 0.03 mmol, 0.1 equiv.) were added. The resulting solution was stirred for 6 hours at 90° C. and the concentrated under vacuum. The residue was purified by flash column chromatography on silica gel, eluting with ethyl acetate/petroleum ether (1:3) to give the crude product, which was further purified by Prep-HPLC with the following condition: Column, XBridge Shield RP18 OBD Column, Sum, 19*150 mm; mobile phase, Water (10 MMOL/L NH₄HCO₃+0.1% NH₃.H₂O) and ACN (55% Phase B up to 75% in 7 min); Detector, uv 254 nm. 7-(cyclohex-1-en-1-yl)-N-(1H-indol-6-yl)quinolin-2-amine (11.9 mg, 11.7%) was isolated as a yellow solid. LCMS Method CD: [M+H]⁺=340. ¹H NMR (400 MHz, DMSO-d₆) δ 10.96 (s, 1H), 9.26 (s, 1H), 8.60 (s, 1H), 7.95 (d, 1H), 7.64-7.62 (m, 2H), 7.45-7.38 (m, 2H), 7.23-7.17 (m, 2H), 7.00 (d, 1H), 6.35 (s, 2H), 2.51-2.49 (m, 2H), 2.26-2.24 (m, 2H), 1.83-1.76 (m, 2H), 1.69-1.65 (m, 2H).

### Step 2: 7-cyclohexyl-N-(1H-indol-6-yl)quinolin-2-amine

7-(Cyclohex-1-en-1-yl)-N-(1H-indol-6-yl)quinolin-2-amine (95.0 mg, 0.3 mmol, 1.0 equiv.) was dissolved in MeOH (5.0 mL), Pd/C (8.9 mg, 0.1 mmol, 0.3 equiv.) was added. The mixture was degassed and back filled with hydrogen, then stirred for 3 hours at ambient temperature under atmosphere of hydrogen. The solids were filtered out, and the filtrate was concentrated under vacuum. The crude product was purified by Prep-HPLC with the following conditions: Column, YMC-Actus Triart C18, 30*250, Sum; mobile phase, Water (10 MMOL/L NH₄HCO₃+0.1% NH₃.H₂O) and ACN (63% Phase B up to 79% in 7 min); Detector, uv 254 nm. 7-Cyclohexyl-N-(1H-indol-6-yl)quinolin-2-amine (48.8 mg, 50.9%) was isolated as an off-white solid. LCMS Method CI: [M+H]⁺=342. ¹H NMR (400 MHz, DMSO-d₆) δ 10.96 (s, 1H), 9.24 (s, 1H), 8.59 (s, 1H), 7.94 (d, 1H), 7.61 (d, 1H), 7.49-7.44 (m, 2H), 7.30-7.16 (m, 3H), 7.01 (d, 1H), 6.36 (s, 1H), 2.68-2.62 (m, 1H), 1.99-1.84 (m, 4H), 1.77-1.74 (m, 1H), 1.57-1.48 (m, 4H), 1.42-1.33 (m, 1H).

### Examples 245: N-(1H-indol-6-yl)-N-methyl-7-(trifluoromethyl)quinolin-2-amine (Compound 605)

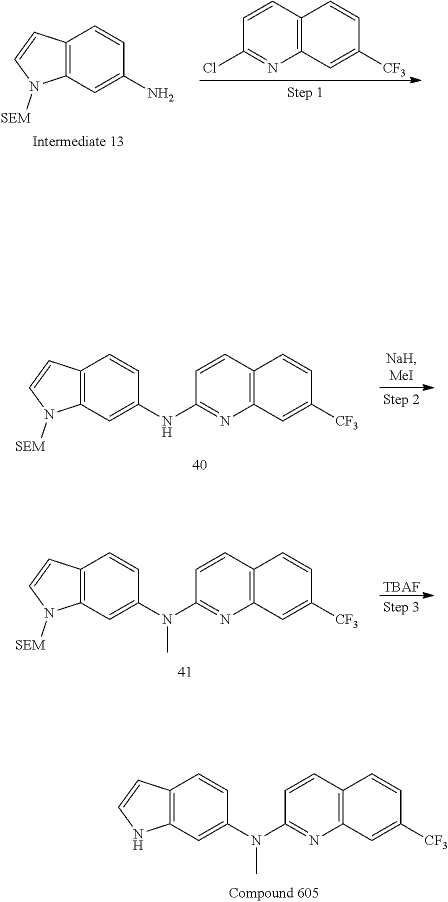 [SMILES: C[Si](C)(C)CCOCn1ccc2ccc(N)cc21 ; FC(F)(F)c1ccc2ccc(Cl)nc2c1 ; C[Si](C)(C)CCOCn1ccc2ccc(Nc3ccc4ccc(C(F)(F)F)cc4n3)cc21 ; CN(c1ccc2ccn(COCC[Si](C)(C)C)c2c1)c1ccc2ccc(C(F)(F)F)cc2n1 ; "TBAF" ; CN(c1ccc2cc[nH]c2c1)c1ccc2ccc(C(F)(F)F)cc2n1]

### Step 1: 7-(trifluoromethyl)-N-(1-[[2-(trimethylsilyl)ethoxy]methyl]indol-6-yl)quinolin-2-amine

1-[[2-(trimethylsilyl)ethoxy]methyl]indol-6-amine (400.0 mg, 1.5 mmol, 1.0 equiv.) was dissolved in dioxane (5.0 mL), 2-chloro-7-(trifluoromethyl)quinoline (353.0 mg, 1.5 mmol, 1.0 equiv.), Cs₂CO₃ (993.3 mg, 3.0 mmol, 2.0 equiv.), Xphos (72.7 mg, 0.15 mmol, 0.1 equiv.), Pd₂(dba)₃ (139.6 mg, 0.15 mmol, 0.1 equiv.) were added. The resulting solution was stirred for 6 hours at 90° C. and then concentrated under vacuum. The residue was purified by flash column chromatography on silica gel, eluting with ethyl acetate/petroleum ether (1:4) to give 7-(trifluoromethyl)-N-(1-[[2-(trimethylsilyl)ethoxy]methyl]indol-6-yl)quinolin-2-amine (600 mg, 86.0%) was isolated as a light yellow solid. LCMS Method CA: [M+H]⁺=458.

### Step 2: N-methyl-7-(trifluoromethyl)-N-(1-[[2-(trimethylsilyl)ethoxy]methyl]indol-6-yl)quinolin-2-amine

7-(Trifluoromethyl)-N-(1-[[2-(trimethylsilyl)ethoxy]methyl]indol-6-yl)quinolin-2-amine (600.0 mg, 1.3 mmol, 1.0 equiv.) was dissolved in THF (20.0 mL), NaH (60% wt in mineral oil, 104.9 mg, 2.6 mmol, 2.0 equiv.) was added. After stirring for 30 min, CH₃I (744.5 mg, 5.2 mmol, 4.0 equiv.) was added. The resulting solution was stirred for additional 1 hour at ambient temperature and then quenched by the addition of water. The resulting solution was extracted with ethyl acetate, dried over anhydrous sodium sulfate and concentrated under vacuum. This resulted in N-methyl-7-(trifluoromethyl)-N-(1-[[2-(trimethylsilyl)ethoxy]methyl]indol-6-yl)quinolin-2-amine (600 mg, 97.0%) as a dark yellow solid. LCMS Method CA: [M+H]⁺=472.

### Step 3: N-(1H-indol-6-yl)-N-methyl-7-(trifluoromethyl)quinolin-2-amine

N-methyl-7-(trifluoromethyl)-N-(1-[[2-(trimethylsilyl)ethoxy]methyl]indol-6-yl)quinolin-2-amine (200.0 mg, 0.4 mmol, 1.0 equiv.) was dissolved in DMF (5.0 mL), ethylenediamine (51.0 mg, 0.8 mmol, 2.0 equiv.) and TBAF (221.8 mg, 0.8 mmol, 2.0 equiv.) were added. The resulting solution was stirred for 5 hours at 80° C. and then diluted with water. The resulting solution was extracted with ethyl acetate, washed with brine, dried over anhydrous sodium sulfate and concentrated under vacuum. The crude product was purified by Prep-HPLC with the following condition: Column, XBridge Shield RP18OBD Column, Sum, 19*150 mm; mobile phase, Water (10 MMOL/L NH₄HCO₃+0.1% NH₃.H₂O) and ACN (55% Phase B up to 78% in 10 min); Detector, uv 254 nm. This resulted in N-(1H-indol-6-yl)-N-methyl-7-(trifluoromethyl)quinolin-2-amine (95.4 mg, 64.6%) as a light yellow solid. LCMS Method CE: [M+H]⁺=342. ¹H NMR (400 MHz, DMSO-d₆) δ 11.23 (s, 1H), 7.95-7.88 (m, 3H), 7.67 (d, 1H), 7.48-7.43 (m, 2H), 7.37 (s, 1H), 6.97 (d, 1H), 6.75 (d, 1H), 6.50 (s, 1H), 3.58 (s, 3H).

### Example 249: Synthesis of 6-((1H-indol-6-yl)amino)-2-methylnicotinonitrile (Compound 598)

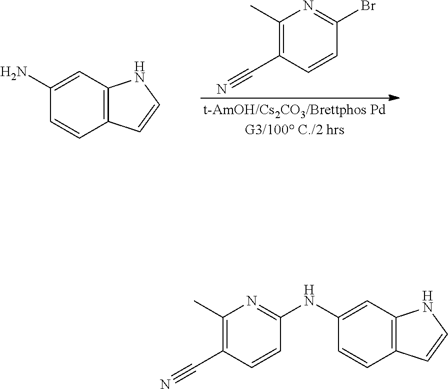

1H-indol-6-amine (53.2 mg, 0.4 mmol, 1.0 equiv.) and 2-chloro-4-(2,2,2-trifluoroethoxy) pyrimidine (79.0 mg, 0.4 mmol, 1.0 equiv.) were dissolved in t-AmOH (3.0 mL), then Cs₂CO₃ (260 mg, 0.8 mmol, 2.0 equiv.) and Brettphos Pd G3 (16.92 mg, 0.02 mmol, 0.05 equiv.) were added under N2 atmosphere. The mixture was stirred at 100° C. for 2 hours. 3.0 mL water was added to the reaction mixture and extracted with EtOAc. The organic layer was collected and concentrated solvent by Speedvac. The residue was purified by prep HPLC to give 6-((1H-indol-6-yl)amino)-2-methylnicotinonitrile (49.02 mg, 0.198 mmol) as solid. MS-ESI, 249.2 [M+H⁺].

¹H NMR (400 MHz, DMSO-d₆) δ ppm 11.01 (br s, 1H) 9.56 (s, 1H) 7.92 (s, 1H) 7.74 (d, 1H) 7.46 (d, 1H) 7.26 (t, 1H) 7.07 (dd, 1H) 6.68 (d, 1H) 6.36 (br s, 1H) 2.52-2.56 (m, 3H)

The compounds in the following table were prepared using the above procedure.

| | | | | LC-MS, |
|---|---|---|---|---|
| Example | Compound | | | MS-ESI, -- |
| # | # | Final compound | IUPAC Name | [M + H⁺]. |
| 250 | 603 | 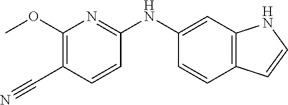 [SMILES: COc1nc(Nc2ccc3cc[nH]c3c2)ccc1C#N] | 6-[(1H-indol-6- yl)amino]-2- methoxypyridine-3- carbonitrile | 265.1 |
| 251 | 585 | 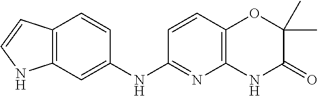 [SMILES: CC1(C)Oc2ccc(Nc3ccc4cc[nH]c4c3)nc2NC1=O] | 6-[(1H-indol-6- yl)amino]-2,2-dimethyl- 2H,3H,4H-pyrido[3,2- b][1,4]oxazin-3-one | 308.4 |
| 253 | 573 | 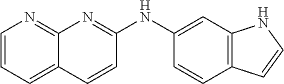 [SMILES: c1cnc2nc(Nc3ccc4cc[nH]c4c3)ccc2c1] | N-(1H-indol-6-yl)-1,8- naphthyridin-2-amine | 261.1 |
| 254 | 601 | 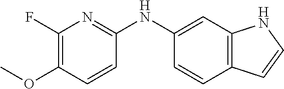 [SMILES: COc1ccc(Nc2ccc3cc[nH]c3c2)nc1F] | N-(6-fluoro-5- methoxypyridin-2-yl)- 1H-indol-6-amine | 258.2 |
| 255 | 561 | 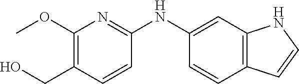 [SMILES: COc1nc(Nc2ccc3cc[nH]c3c2)ccc1CO] | {6-[(1H-indol-6-yl)amino]- 2-methoxypyridin-3- yl}methanol | 270 |
| 256 | 600 | 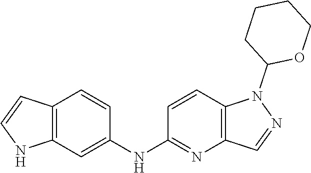 [SMILES: c1cc2ccc(Nc3ccc4c(cnn4C4CCCCO4)n3)cc2[nH]1] | N-[1-(oxan-2-yl)-1H- pyrazolo[4,3-b]pyridin-5- yl]-1H-indol-6-amine | 334.2 |
| 257 | 599 | 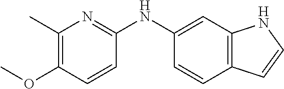 [SMILES: COc1ccc(Nc2ccc3cc[nH]c3c2)nc1C] | N-(5-methoxy-6- methylpyridin-2-yl)-1H- indol-6-amine | 254.1 |
| 258 | 656 | 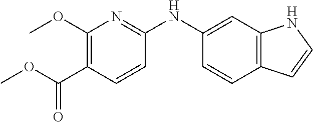 [SMILES: COC(=O)c1ccc(Nc2ccc3cc[nH]c3c2)nc1OC] | methyl 6-[(1H-indol-6- yl)amino]-2- methoxypyridine-3- carboxylate | 298.1 |
| 259 | 596 | 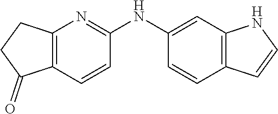 [SMILES: O=C1CCc2nc(Nc3ccc4cc[nH]c4c3)ccc21] | 2-[(1H-indol-6- yl)amino]-5H,6H,7H- cyclopenta[b]pyridin-5-one | 264.2 |
| 260 | 595 | 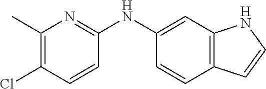 [SMILES: Cc1nc(Nc2ccc3cc[nH]c3c2)ccc1Cl] | N-(5-chloro-6- methylpyridin-2-yl)-1H- indol-6-amine | 258.1 |
| 262 | 571 | 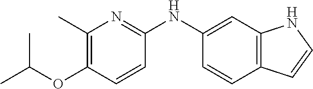 [SMILES: Cc1nc(Nc2ccc3cc[nH]c3c2)ccc1OC(C)C] | N-[6-methyl-5-(propan-2- yloxy)pyridin-2-yl]-1H- indol-6-amine | 282.2 |
| 263 | 594 | 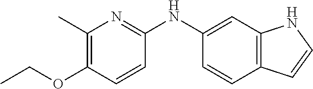 [SMILES: CCOc1ccc(Nc2ccc3cc[nH]c3c2)nc1C] | N-(5-ethoxy-6- methylpyridin-2-yl)-1H- indol-6-amine | 268.1 |
| 264 | 584 | 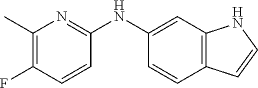 [SMILES: Cc1nc(Nc2ccc3cc[nH]c3c2)ccc1F] | N-(5-fluoro-6- methylpyridin-2-yl)-1H- indol-6-amine | 242.1 |
| 265 | 583 | 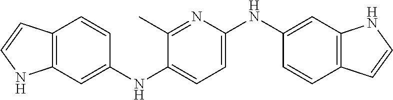 [SMILES: Cc1nc(Nc2ccc3cc[nH]c3c2)ccc1Nc1ccc2cc[nH]c2c1] | N2,N5-bis(1H-indol-6- yl)-6-methylpyridine-2,5- diamine | 354.2 |
| 266 | 593 | 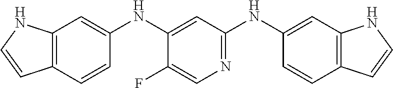 [SMILES: Fc1cnc(Nc2ccc3cc[nH]c3c2)cc1Nc1ccc2cc[nH]c2c1] | 5-fluoro-N2,N4-bis(1H- indol-6-yl)pyridine-2,4- diamine | 358.1 |
| 267 | 582 | 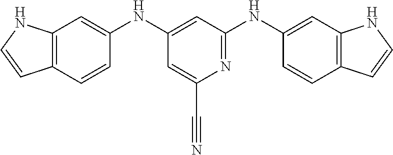 [SMILES: N#Cc1cc(Nc2ccc3cc[nH]c3c2)cc(Nc2ccc3cc[nH]c3c2)n1] | 4,6-bis[(1H-indol-6- yl)amino]pyridine-2- carbonitrile | 365.1 |
| 268 | 570 | 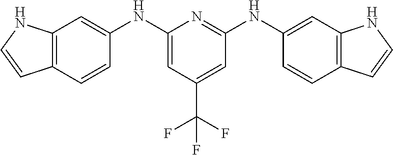 [SMILES: FC(F)(F)c1cc(Nc2ccc3cc[nH]c3c2)nc(Nc2ccc3cc[nH]c3c2)c1] | N2,N6-bis(1H-indol-6- yl)-4- (trifluoromethyl)pyridine- 2,6-diamine | 408.1 |
| 269 | 581 | 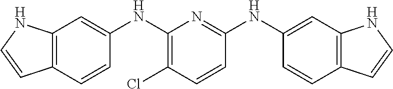 [SMILES: Clc1ccc(Nc2ccc3cc[nH]c3c2)nc1Nc1ccc2cc[nH]c2c1] | 3-chloro-N2,N6-bis(1H- indol-6-yl)pyridine-2,6- diamine | 374.2 |
| 270 | 560 | 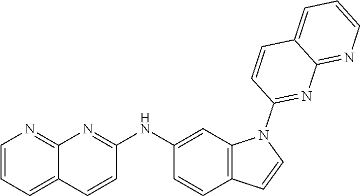 [SMILES: c1cnc2nc(Nc3ccc4ccn(-c5ccc6cccnc6n5)c4c3)ccc2c1] | N-[1-(1,8-naphthyridin-2- yl)-1H-indol-6-yl]-1,8- naphthyridin-2-amine | 389.2 |
| 271 | 569 | 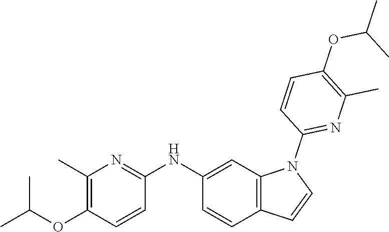 [SMILES: Cc1nc(Nc2ccc3ccn(-c4ccc(OC(C)C)c(C)n4)c3c2)ccc1OC(C)C] | N,1-bis[6-methyl-5- (propan-2-yloxy)pyridin- 2-yl]-1H-indol-6-amine | 431.3 |

### Example 272: Synthesis of 4-chloro-N-(1H-indol-6-yl)-7-(trifluoromethyl)quinolin-2-amine (Compound 562)

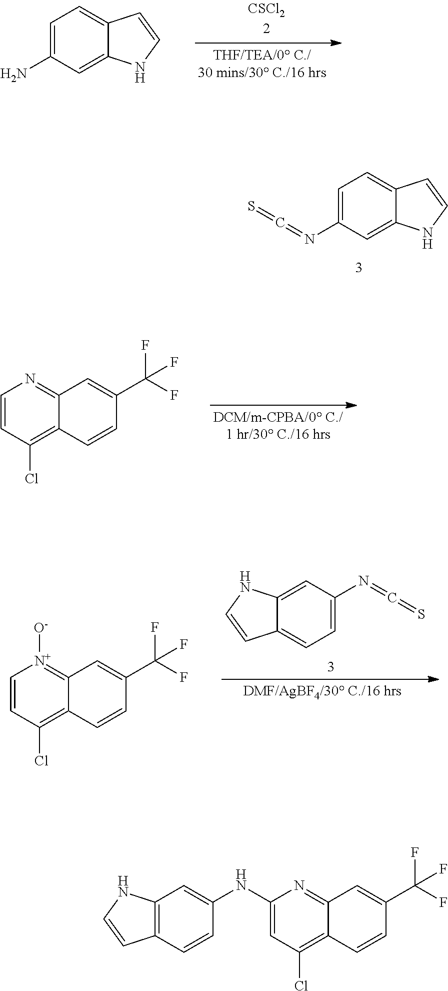 [SMILES: Nc1ccc2cc[nH]c2c1 ; S=C=Nc1ccc2cc[nH]c2c1 ; FC(F)(F)c1ccc2c(Cl)ccnc2c1 ; [O-][n+]1ccc(Cl)c2ccc(C(F)(F)F)cc21 ; S=C=Nc1ccc2cc[nH]c2c1 ; FC(F)(F)c1ccc2c(Cl)cc(Nc3ccc4cc[nH]c4c3)nc2c1]

Step 1:1H-indol-6-amine (3.014 g, 23.0 mmol, 1.0 equiv.) and thiophosgene (3.86 g, 34.0 mmol, 1.5 equiv.) and TEA (1.668 g, 114.0 mmol, 5.0 equiv.) were dissolved in THF (50 mL). The mixture was stirred at 0° C. for 30 mins, then the mixture was stirred at 30° C. for 16 hours. 50.0 mL water was added to the reaction mixture and extracted with EtOAc. The organic layer was collected and concentrated by Speedvac. The residue was purified by flash silica gel chromatography (ISCO®; 80 g SepaFlash® Silica Flash Column, Eluent of 0˜10% Ethyl acetate/Petroleum ethergradient @ 100 mL/min). to give 6-isothiocyanato-1H-indole (1.50 g, 8.62 mmol) as solid.

Step 2:4-chloro-7-(trifluoromethyl)quinolone (352.9 mg, 1.5 mmol, 1.0 equiv.) and m-CPBA (519.2 mg, 2.3 mmol, 1.5 equiv.) were dissolved in DCM (3.0 mL). The mixture was stirred at 0° C. for 1 hour. 3.0 mL Na₂SO₃ was added to the reaction mixture and extracted with DCM, dried over anhydrous Na₂SO₄. The organic layer was collected and concentrated by Speedvac. The residue was purified by TLC to give 6-(trifluoromethyl)quinoxaline 1-oxide (196.2 mg, 0.9 mmol) as solid.

Step 3:6-(trifluoromethyl)quinoxaline (196.2 mg, 0.9 mmol, 1.0 equiv.) and 6-isothiocyanato-1H-indole (239 mg, 1.4 mmol, 1.0 equiv.) and AgBF₄ (35.3 mg, 0.2 mmol, 0.2 equiv.) were dissolved in ME (3.0 mL). The mixture was stirred at 30UPC for 16 hours. 3.0 mL water was added to the reaction mixture and extracted with EtOAc. The organic layer was collected and concentrated by Speedvac. The residue was purified by prep HPLC to give 4-chloro-N-(1H-indol-6-yl)-7-(trifluoromethyl)quinolin-2-amine (22.4 mg, 0.06 mmol) as solid. MS-ESI, 362.2 [M+H⁺].

¹H NMR (400 MHz, DMSO-d₆) δ ppm 11.03 (br s, 1H) 9.74 (br s, 1H) 8.51 (s, 1H) 8.16 (d, 1H) 7.99 (s, 1H) 7.65 (br d, 1H) 7.50 (d, 1H) 7.41 (s, 1H) 7.26-7.32 (m, 1H) 7.17 (dd, 1H) 6.39 (br s, 1H)

The following examples were synthesized using methods similar to the above.

| | | | | LC-MS, |
|---|---|---|---|---|
| Example | Compound | | IUPAC | MS-ESI, -- |
| # | # | Final compound | Name | [M + H+]. |
| 273 | 588 | 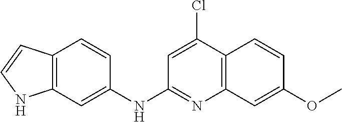 [SMILES: COc1ccc2c(Cl)cc(Nc3ccc4cc[nH]c4c3)nc2c1] | 4-chloro-N-(1H- indol-6-yl)-7- methoxyquinolin- 2-amine | 324.1 |
| 274 | 589 | 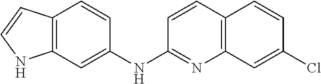 [SMILES: Clc1ccc2ccc(Nc3ccc4cc[nH]c4c3)nc2c1] | 7-chloro-N-(1H- indol-6-yl)quinolin- 2-amine | 294.1 |
| 275 | 587 | 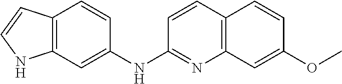 [SMILES: COc1ccc2ccc(Nc3ccc4cc[nH]c4c3)nc2c1] | N-(1H-indol-6-yl)- 7-methoxyquinolin- 2-amine | 290.1 |
| 276 | 577 | 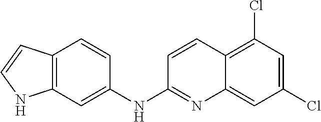 [SMILES: Clc1cc(Cl)c2ccc(Nc3ccc4cc[nH]c4c3)nc2c1] | 5,7-dichloro-N- (1H-indol-6- yl)quinolin-2-amine | 328.1 |
| 277 | 576 | 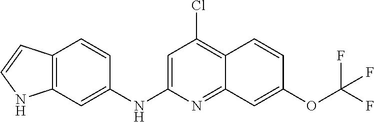 [SMILES: FC(F)(F)Oc1ccc2c(Cl)cc(Nc3ccc4cc[nH]c4c3)nc2c1] | 4-chloro-N-(1H- indol-6-yl)-7- (trifluoromethoxy) quinolin-2-amine | 377.7 |
| 278 | 586 | 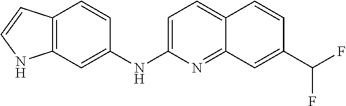 [SMILES: FC(F)c1ccc2ccc(Nc3ccc4cc[nH]c4c3)nc2c1] | 7-(difluoromethyl)- N-(1H-indol-6- yl)quinolin-2-amine | 310.1 |
| 279 | 575 | 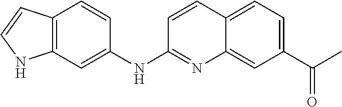 [SMILES: CC(=O)c1ccc2ccc(Nc3ccc4cc[nH]c4c3)nc2c1] | 1-{2-[(1H-indol-6- yl)amino]quinolin- 7-yl}ethan-1-one | 301.9 |
| 280 | 574 | 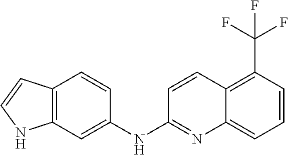 [SMILES: FC(F)(F)c1cccc2nc(Nc3ccc4cc[nH]c4c3)ccc12] | N-(1H-indol-6-yl)- 5-(trifluoromethyl) quinolin-2-amine | 327.8 |

### Biological Assays

STING pathway activation by the compounds described herein were measured using THP1-Dual™ cells (KO-IFNAR2).

THP1-Dual™ KO-IFNAR2 Cells (obtained from invivogen) were maintained in RPMI, 10% FCS, 5 ml P/S, 2 mM L-glut, 10 mM Hepes, and 1 mM sodium pyruvate. Compounds were spotted in empty 384 well tissue culture plates (Greiner 781182) by Echo for a final concentration of 0.0017-100 μM. Cells were plated into the TC plates at 40 μL per well, 2×10E6 cells/mL. For activation with STING ligand, 2′3′cGAMP (MW 718.38, obtained from Invivogen), was prepared in Optimem media.

The following solutions were prepared for each 1×384 plate:
Solution A: 2 mL Optimem with one of the following stimuli:
   60 uL of 10 mM 2′3′cGAMP ->150 μM stock
Solution B: 2 mL Optimem with 60 μL Lipofectamine 2000->Incubate 5 min at RT

2 mL of solution A and 2 ml Solution B was mixed and incubated for 20 min at room temperature (RT). 20 uL of transfection solution (A+B) was added on top of the plated cells, with a final 2′3′cGAMP concentration of 15 μM. The plates were then centrifuged immediately at 340 g for 1 minute, after which they were incubated at 37° C., 5% CO₂, >98% humidity for 24h. Luciferase reporter activity was then measured. EC₅₀ values were calculated by using standard methods known in the art.

Luciferase reporter assay: 10 μL of supernatant from the assay was transferred to white 384-plate with flat bottom and squared wells. One pouch of QUANTI-Luc™ Plus was dissolved in 25 mL of water. 100 μL of QLC Stabilizer per 25 mL of QUANTI-Luc™ Plus solution was added. 50 μL of QUANTI-Luc™ Plus/QLC solution per well is then added. Luminescence is measured on a Platereader (e.g., Spectramax I3X (Molecular Devices GF3637001)).

Luciferase reporter activity was then measured. EC₅₀ values were calculated by using standard methods known in the art.

Table BA shows the activity of compounds in STING reporter assay: <0.008 μM=“++++++”; ≥0.008 and <0.04 μM=“+++++”; ≥0.04 and <0.2 M=“++++”; ≥0.2 and <1 μM=“+++”; ≥1 and <5 μM=“++”; ≥5 and <100 μM=“+”.

TABLE BA

| Compound | Human STING Reporter | Compound | Human STING Reporter |
|---|---|---|---|
| # | Assay EC50 (μM) | # | Assay EC50 (μM) |
| 101 | ++++ | 355 | >30.0000 |
| 102 | ++ | 356 | >30.0000 |
| 110 | + | 357 | >30.0000 |
| 111 | +++ | 358 | >30.0000 |
| 113 | >14.7737 | 359 | >30.0000 |
| 151 | + | 360 | >30.0000 |
| 152 | +++ | 361 | >30.0000 |
| 153 | >100.00 | 362 | >30.0000 |
| 154 | >100.00 | 363 | >30.0000 |
| 153 | +++ | 364 | >30.0000 |
| 154 | >30.0000 | 365 | >30.0000 |
| 155 | ++++ | 366 | >30.0000 |
| 156 | >30.0000 | 370 | + |
| 157 | >30.0000 | 371 | +++ |
| 159 | >30.0000 | 372 | >100.0000 |
| 160 | ++ | 373 | >100.0000 |
| 161 | >30.0000 | 374 | + |
| 162 | + | 375 | >100.0000 |
| 164 | +++ | 376 | + |
| 165 | +++ | 377 | >100.0000 |
| 166 | >30.0000 | 378 | ++ |
| 167 | ++ | 379 | >5 |
| 168 | + | 381 | +++ |
| 169 | ++ | 384 | ++ |
| 170 | ++ | 385 | ++ |
| 171 | >30.000 | 386 | ++ |
| 172 | ++ | 387 | ++ |
| 173 | +++ | 388 | ++ |
| 174 | + | 389 | ++ |
| 175 | ++ | 391 | + |
| 176 | ++ | 392 | >30.0000 |
| 177 | ++ | 393 | >30.0000 |
| 178 | >30.0000 | 394 | >30.0000 |
| 179 | >30.0000 | 395 | >30.0000 |
| 180 | >30.0000 | 396 | + |
| 181 | ++ | 397 | >30.0000 |
| 182 | +++ | 398 | >30.0000 |
| 183 | +++ | 399 | >30.0000 |
| 184 | >30.0000 | 400 | >30.0000 |
| 185 | >30.0000 | 401 | ++ |
| 186 | ++ | 402 | + |
| 187 | >30.0000 | 403 | + |
| 188 | ++ | 404 | >30.0000 |
| 189 | >30.0000 | 405 | +++ |
| 190 | >30.0000 | 406 | + |
| 191 | >30.0000 | 407 | >30.0000 |
| 192 | >30.0000 | 408 | + |
| 193 | >30.0000 | 409 | >30.0000 |
| 194 | >30.0000 | 410 | >30.0000 |
| 195 | ++ | 411 | >30.0000 |
| 196 | +++ | 412 | + |
| 197 | >30.0000 | 413 | >30.0000 |
| 198 | + | 414 | >30.0000 |
| 199 | ++ | 415 | >30.0000 |
| 200 | +++ | 416 | >30.0000 |
| 201 | ++ | 418 | + |
| 202 | + | 419 | >30.0000 |
| 203 | ++ | 420 | ++ |
| 204 | ++ | 421 | ++ |
| 205 | +++ | 422 | + |
| 206 | ++ | 423 | >30.0000 |
| 207 | ++ | 424 | ++ |
| 208 | +++ | 425 | >30.0000 |
| 209 | ++ | 426 | + |
| 210 | ++ | 427 | >20.4219 |
| 211 | ++ | 428 | + |
| 212 | ++ | 429 | + |
| 213 | >30.0000 | 430 | >30.0000 |
| 214 | ++ | 431 | + |
| 215 | ++ | 432 | >30.0000 |
| 216 | >30.0000 | 433 | >30.0000 |
| 217 | + | 478 | >30.0000 |
| 218 | >30.0000 | 479 | |
| 219 | >30.0000 | 480 | |
| 220 | >30.0000 | 481 | |
| 221 | >30.0000 | 482 | |
| 222 | + | 483 | |
| 223 | >30.0000 | 484 | |
| 224 | ++ | 485 | |
| 225 | + | 486 | |
| 226 | +++ | 487 | |
| 227 | +++ | 488 | |
| 228 | ++ | 489 | |
| 229 | >30.0000 | 490 | |
| 230 | >30.0000 | 491 | |
| 231 | >30.0000 | 492 | |
| 233 | +++ | 493 | |
| 234 | +++ | 494 | |
| 235 | +++ | 495 | |
| 236 | +++ | 496 | |
| 237 | +++ | 497 | |
| 240 | +++ | 498 | >30.0000 |
| 242 | +++ | 499 | >1.1110 |
| 243 | + | 500 | +++ |
| 244 | +++ | 501 | ++ |
| 245 | ++++ | 502 | |
| 246 | +++ | 503 | +++ |
| 247 | ++ | 504 | +++ |
| 248 | +++ | 505 | ++ |
| 249 | ++ | 506 | + |
| 250 | ++ | 507 | ++ |
| 251 | ++++ | 508 | + |
| 252 | ++++ | 509 | ++ |
| 253 | ++++ | 510 | ++ |
| 254 | +++ | 511 | >30.0000 |
| 255 | + | 512 | ++ |
| 256 | >100.0000 | 513 | + |
| 257 | ++ | 514 | +++ |
| 258 | +++ | 515 | >30.0000 |
| 259 | >30.000 | 516 | +++ |
| 260 | + | 517 | >30.0000 |
| 261 | >100.0000 | 518 | ++++ |
| 262 | + | 519 | + |
| 263 | + | 520 | +++ |
| 264 | + | 521 | ++ |
| 265 | + | 522 | >30.0000 |
| 266 | >100.0000 | 523 | ++ |
| 267 | >67.0923 | 524 | ++ |
| 268 | + | 525 | +++ |
| 269 | +++ | 526 | >7.3838 |
| 270 | ++++ | 527 | + |
| 271 | +++ | 528 | ++ |
| 272 | +++ | 529 | ++ |
| 273 | +++ | 530 | >30.0000 |
| 274 | >30.0000 | 531 | +++ |
| 275 | ++ | 532 | >30.0000 |
| 276 | >30.0000 | 533 | ++ |
| 277 | | 534 | +++ |
| 278 | >30.0000 | 535 | +++ |
| 279 | + | 536 | +++ |
| 282 | >30.0000 | 537 | +++ |
| 283 | >30.0000 | 538 | + |
| 284 | >30.0000 | 539 | + |
| 285 | >30.0000 | 540 | + |
| 286 | >30.0000 | 541 | >30.0000 |
| 287 | >30.0000 | 542 | +++ |
| 288 | >30.0000 | 543 | + |
| 289 | + | 544 | >30.0000 |
| 290 | >30.0000 | 545 | >30.0000 |
| 291 | ++ | 546 | + |
| 292 | >30.0000 | 547 | + |
| 293 | >30.0000 | 548 | ++++ |
| 294 | >30.0000 | 549 | +++ |
| 295 | >30.0000 | 550 | +++ |
| 296 | >30.0000 | 551 | +++ |
| 297 | >30.0000 | 552 | +++ |
| 298 | >30.0000 | 553 | +++ |
| 299 | >30.0000 | 554 | ++ |
| 300 | >30.0000 | 555 | ++ |
| 301 | >30.0000 | 556 | ++ |
| 302 | + | 557 | >30.0000 |
| 303 | >30.0000 | 558 | +++ |
| 304 | >30.0000 | 559 | >30.0000 |
| 305 | >30.0000 | 560 | ++ |
| 306 | >30.0000 | 561 | >30.0000 |
| 307 | >30.0000 | 562 | + |
| 308 | >30.0000 | 563 | +++ |
| 309 | + | 564 | +++ |
| 311 | >30.0000 | 565 | +++ |
| 312 | >30.0000 | 566 | >30.0000 |
| 313 | + | 567 | + |
| 314 | >30.0000 | 568 | +++ |
| 315 | + | 569 | >30.0000 |
| 316 | >30.0000 | 570 | +++ |
| 317 | >30.0000 | 571 | ++ |
| 318 | + | 572 | + |
| 319 | >30.0000 | 573 | + |
| 320 | >30.0000 | 574 | +++ |
| 321 | >30.0000 | 575 | >30.0000 |
| 322 | >30.0000 | 576 | ++ |
| 323 | >30.0000 | 577 | +++ |
| 324 | >30.0000 | 578 | +++ |
| 325 | >30.0000 | 579 | ++ |
| 326 | >30.0000 | 580 | + |
| 327 | >30.0000 | 581 | + |
| 328 | >30.0000 | 582 | +++ |
| 329 | >30.0000 | 583 | + |
| 330 | >30.0000 | 584 | >30.0000 |
| 331 | >30.0000 | 585 | >30.0000 |
| 332 | >30.0000 | 586 | ++ |
| 333 | >30.0000 | 587 | ++ |
| 334 | >30.0000 | 588 | ++ |
| 335 | >30.0000 | 589 | +++ |
| 336 | >30.0000 | 590 | +++ |
| 337 | >30.0000 | 591 | ++ |
| 338 | >30.0000 | 592 | ++ |
| 339 | >30.0000 | 593 | ++ |
| 340 | >30.0000 | 594 | + |
| 341 | >30.0000 | 595 | + |
| 342 | >30.0000 | 596 | >30.0000 |
| 343 | + | 597 | >30.0000 |
| 344 | + | 598 | >30.0000 |
| 345 | + | 599 | >30.0000 |
| 346 | >30.0000 | 600 | >30.0000 |
| 347 | >30.0000 | 601 | >30.0000 |
| 348 | >30.0000 | 602 | ++ |
| 349 | >30.0000 | 603 | >30.0000 |
| 350 | >30.0000 | 604 | +++ |
| 351 | >30.0000 | 605 | + |
| 352 | >30.0000 | 606 | ++ |
| 353 | >30.0000 | 607 | +++ |
| 354 | >30.0000 | 608 | + |
| 618 | ++ | 609 | + |
| 619 | ++ | 620 | ++ |

### Numbered Clauses

The compounds, compositions, methods, and other subject matter described herein are further described in the following numbered clauses:

1. A compound of Formula I:

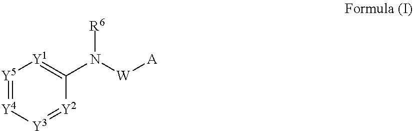

or a pharmaceutically acceptable salt thereof or a tautomer thereof,

wherein:

each of Y¹, Y², Y³, Y⁴, and Y⁵ is independently selected from the group consisting of N and CR¹;

W-A is defined according to (A) or (B) below:
(A)

W is selected from the group consisting of:

(f) *C(═O)NR^{N}, *C(═S)NR^{N}, *C(═NR^{N})NR^{N} (e.g., *C(═NCN)NR^{N}), *C(═CNO₂)NR^{N}

(g) *S(O)₁₋₂NR^{N};

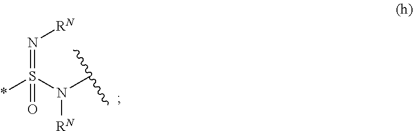

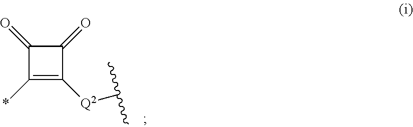

and

(j) *Q¹-Q²;

wherein the asterisk denotes point of attachment to NR⁶;

Q¹ is selected from the group consisting of:

(c) phenylene optionally substituted with from 1-2 independently selected R^{q1}; and

(d) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};

Q² is selected from the group consisting of: a bond, NR^{N}, —S(O)₀₋₂—, —O—, and —C(═O)—;

A is:

(i) —Y^{A1}-Y^{A2}, wherein:
Y^{A1} is a bond; or
Y^{A1} is C₁₋₆ alkylene, which is optionally substituted with from 1-6 substituents each independently selected from the group consisting of:
R^{a};
C₆₋₁₀ aryl optionally substituted with 1-4 independently selected C₁₋₄ alkyl; and
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; or
Y^{A1} is —Y^{A3}-Y^{A4}—Y^{A5} which is connected to W via Y^{A3} wherein:
Y^{A3} is a C₁₋₃ alkylene optionally substituted with from 1-2 independently selected R^{a};
Y^{A4} is —O—, —NH—, or —S—; and
Y^{A} is a bond or C₁₋₃ alkylene which is optionally substituted with from 1-2 independently selected R^{a}; and
Y^{A2} is:

(a) C₃₋₂₀ cycloalkyl, which is optionally substituted with from 1-4 R^{b},

(b) C₆₋₂₀ aryl, which is optionally substituted with from 1-4 R^{c};

(c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or

(d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},

OR

(ii) —Z¹-Z²-Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};

OR

(iii) C₁₋₂₀ alkyl, which is optionally substituted with from 1-6 independently selected R^{a},

OR
(B)

W is selected from the group consisting of:

(a) C₈₋₂₀ bicyclic or polycyclic arylene, which is optionally substituted with from 1-4 R^{c}; and

(b) bicyclic or polycyclic heteroarylene including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};

A is as defined for (A), or A is H;

each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″), and
—L³-L⁴-L⁵-Rⁱ;

or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;

each R² is independently selected from the group consisting of:
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl) optionally substituted with from 1-3 independently selected R^{a},
—S(O)(═NH)(C₁₋₄ alkyl) optionally substituted with from 1-3 independently selected R^{a},
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl) optionally substituted with from 1-3 independently selected R^{a},
—C(═O)O(C₁₋₄ alkyl) optionally substituted with from 1-3 independently selected R^{a},
—C(═O)OH,
—C(═O)N(R′)(R″); and
—L³-L⁴-L⁵-Rⁱ;

R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;

each occurrence of R^{q1} is independently selected from the group consisting of:

(a) halo;

(b) cyano;

(c) C₁₋₁₀ alkyl which is optionally substituted with from 1-6 independently selected R^{a};

(d) C₂₋₆ alkenyl;

(e) C₂₋₆ alkynyl;

(f) C₃₋₆ cycloalkyl;

(g) C₁₋₄ alkoxy;

(h) C₁₋₄ haloalkoxy;

(i) —S(O)₁₋₂(C₁₋₄ alkyl);

(j) —NR^{e}R^{f};

(k) —OH;

(l) —S(O)₁₋₂(NR′R″);

(m) —C₁₋₄ thioalkoxy;

(n) —NO₂;

(o) —C(═O)(C₁₋₄ alkyl);

(p) —C(═O)O(C₁₋₄ alkyl);

(q) —C(═O)OH;

(r) —C(═O)N(R′)(R″); and

(s) oxo;

each occurrence of R^{a} is independently selected from the group consisting of: —OH; —F; —Cl ; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl; each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl ; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};

each occurrence of R^{c} is independently selected from the group consisting of:

(a) halo;

(b) cyano;

(c) C₁₋₁₀ alkyl which is optionally substituted with from 1-6 independently selected R^{a};

(d) C₂₋₆ alkenyl;

(e) C₂₋₆ alkynyl;

(g) C₁₋₄ alkoxy;

(h) C₁₋₄ haloalkoxy;

(i) —S(O)₁₋₂(C₁₋₄ alkyl) or —S(O)₁₋₂(C₁₋₄ haloalkyl);

(j) —NR^{e}R^{f};

(k) —OH;

(l) —S(O)₁₋₂(NR′R″);

(m) —C₁₋₄ thioalkoxy or —C₁₋₄ thiohaloalkoxy;

(n) —NO₂;

(o) —C(═O)(C₁₋₁₀ alkyl);

(p) —C(═O)O(C₁₋₄ alkyl);

(q) —C(═O)OH;

(r) —C(═O)N(R′)(R″);

(s)-L¹-L²-R^{h}; and

(t) —SF₅

each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;

each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;

-L¹ is a bond or C₁₋₃ alkylene optionally substituted with oxo;

-L² is —O—, —N(H)—, —S(O)₀₋₂—, or a bond;

R^{h} is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;

-L³ is a bond or C₁₋₃ alkylene optionally substituted with oxo;

-L⁴ is a bond; —O—; —N(R^{N})—; —S(O)₀₋₂—; C(═O); —NR^{N}S(O)₀₋₂—; —S(O)₀₋₂NR^{N}—; —NR^{N}S(O)₁₋₂NR^{N}—; —S(═O)(═NR^{N}); —NR^{N}S(═O)(═NR^{N}); —S(═O)(═NR^{N})NR^{N}; NR^{N}S(═O)(═NR^{N})NR^{N}; —NR^{N}C(O)—; —NR^{N}C(O)NR^{N}—; C₃₋₆ cycloalkylene; or heterocyclylene including from 3-8 ring atoms wherein from 1-3 ring atoms are heteroatoms each independently selected from the group consisting of N, NH, N(R^{d}), O, and S(O)₀₋₂;

-L⁵ is a bond or C₁₋₄ alkylene;

Rⁱ is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;

each occurrence of R^{N} is independently H or R^{d}; and

each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₄ alkyl), O, and S, provided that one or more compound provisions herein apply.

2. The compound of clause 1, wherein from 2-5 of Y¹, Y², Y³, Y⁴, and Y⁵ are independently CR¹.

3. The compound of any one of clauses 1-2, wherein the ring including Y¹, Y², Y³, Y⁴, and Y⁵ is selected from the group consisting of:

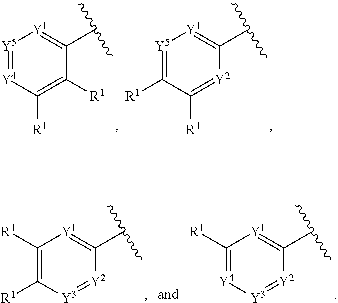

4. The compound of any one of clauses 1-3, wherein each of Y¹, Y², Y³, Y⁴, and Y⁵ is an independently selected CR¹ (i.e., the ring including Y¹, Y², Y³, Y⁴, and Y⁵ is

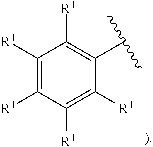

5. The compound of clause 4, wherein the ring including Y¹, Y², Y³, Y⁴, and Y⁵ is selected from the group consisting of:

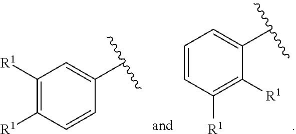

6. The compound of clause 4, wherein the ring including Y¹, Y², Y³, Y⁴, and Y⁵ is selected from the group consisting of:

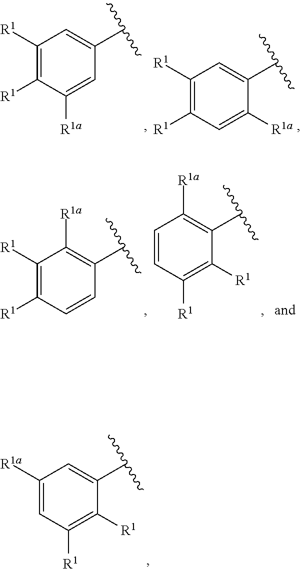

wherein each R^{1a} is an independently selected R¹.

7. The compound of clause 4, wherein the ring including Y¹, Y², Y³, Y⁴, and Y⁵ is selected from the group consisting of:

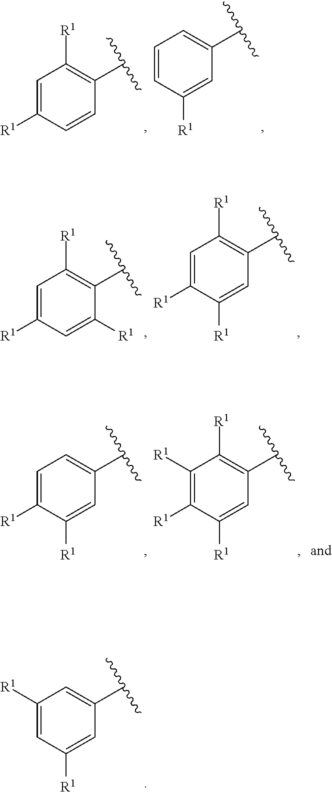

8. The compound of any one of clauses 1-3, wherein from 1-2 (e.g., 1 or 2) of Y¹, Y², Y³, Y⁴, and Y⁵ is independently N; and each of the remaining Y¹, Y², Y³, Y⁴, and Y is an independently selected CR¹.

9. The compound of clause 8, wherein the ring including Y¹, Y², Y³, Y⁴, and Y⁵ is pyridinyl.

10. The compound of clause 9, wherein the ring including Y¹, Y², Y³, Y⁴, and Y⁵ is pyridin-2-yl (i.e.

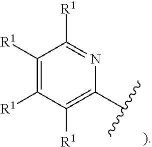

11. The compound of clause 10, wherein the ring including Y¹, Y², Y³, Y⁴, and Y⁵ is selected from the group consisting of:

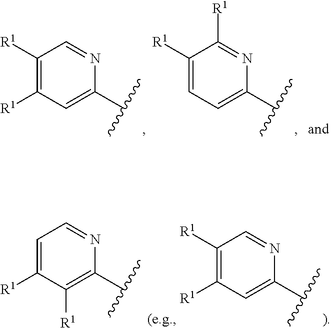

12. The compound of clause 9, wherein the ring including Y¹, Y², Y³, Y⁴, and Y⁵ is pyridin-3-yl (i.e.,

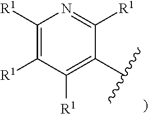

or pyridin-4-yl (i.e.,

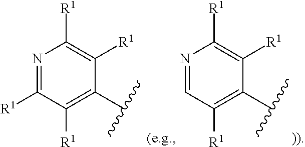

13. The compound of clause 12, wherein the ring including Y¹, Y², Y³, Y⁴, and Y⁵ is selected from the group consisting of:

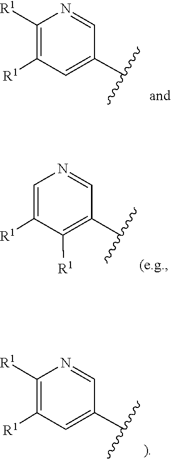

14. The compound of clause 12, wherein the ring including Y¹, Y², Y³, Y⁴, and Y⁵ is selected from the group consisting of:

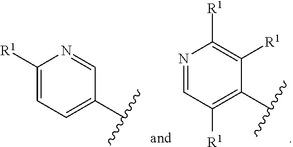

15. The compound of clause 8, wherein the ring including Y¹, Y², Y³, Y⁴, and Y⁵ is pyrimidinyl (e.g.,

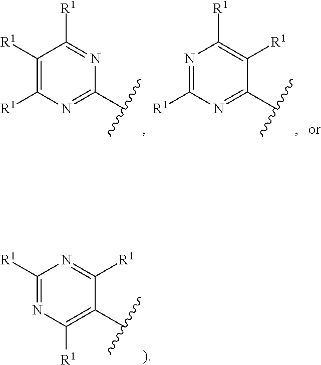

16. The compound of clause 15, wherein the ring including Y¹, Y², Y³, Y⁴, and Y⁵ is

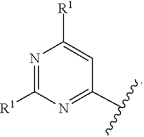

17. The compound of any one of clauses 1-6, 8-13, and 15, wherein a pair of R¹ on adjacent atoms, taken together with the atoms connecting them, form a ring including from 4-15 (e.g., 5-12 (e.g., 5, 6, 7, 8, 9, or 10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

18. The compound of clause 17, wherein a pair of R¹ on adjacent atoms, taken together with the atoms connecting them, form a ring including from 5-12 (e.g., 5, 6, 7, 8, 9, or 10) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

19. The compound of any one of clauses 17-18, wherein a pair of R¹ on adjacent atoms, taken together with the atoms connecting them, form a ring including from 5-6 ring atoms (e.g., an aromatic ring including from 5-6 ring atoms), wherein from 0-2 ring atoms are heteroatoms each independently selected from the group consisting of N, N(H), N(R^{d}), O, and S(O)₀₋₂; and wherein the ring is optionally substituted with from 1-4 independently selected R².

20. The compound of clause 19, wherein a pair of R¹ on adjacent atoms, taken together with the atoms connecting them, form a ring including 5 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

21. The compound of clause 20, wherein a pair of R¹ on adjacent atoms, taken together with the atoms connecting them, form an aromatic ring including 5 ring atoms, wherein from 1-2 (e.g., 1; or e.g., 2) ring atoms are heteroatoms each independently selected from the group consisting of N, N(H), N(R^{d}), O, and S(O)₀₋₂; and wherein the ring is optionally substituted with from 1-4 independently selected R².

22. The compound of clause 21, wherein a pair of R¹ on adjacent atoms, taken together with the atoms connecting them, form a pyrrolyl ring optionally substituted with from 1-2 independently selected R².

23. The compound of clause 22, wherein a pair of R¹ on adjacent atoms, taken together with the atoms connecting them, form:

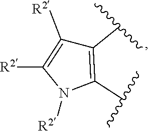

wherein each R^{2′} is independently H or R² (e.g.,

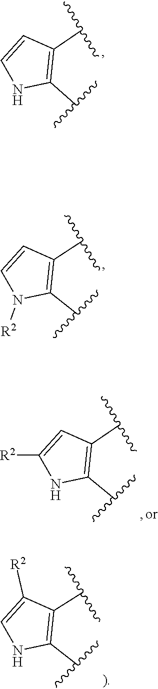

24. The compound of clause 21, wherein a pair of R¹ on adjacent atoms, taken together with the atoms connecting them, form a pyrazolyl, imidazolyl, or thiazolyl ring optionally substituted with from 1-2 independently selected R².

25. The compound of clause 24, wherein a pair of R¹ on adjacent atoms, taken together with the atoms connecting them, form:

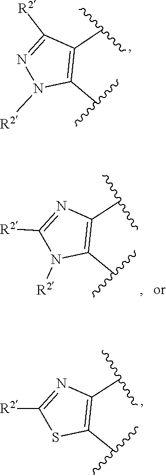

wherein each R^{2′} is independently H or R² (e.g.,

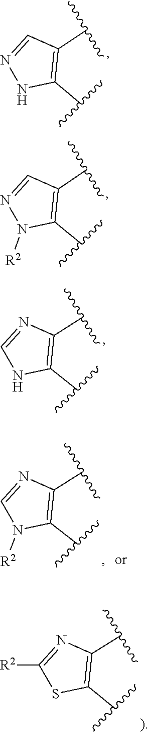

26. The compound of clause 19 wherein a pair of R¹ on adjacent atoms, taken together with the atoms connecting them, form a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

27. The compound of clause 26, wherein a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; and wherein the ring is optionally substituted with from 1-4 independently selected R².

28. The compound of clause 27, wherein a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R².

29. The compound of clause 28, wherein a pair of R¹ on adjacent atoms, taken together with the atoms connecting them, form:

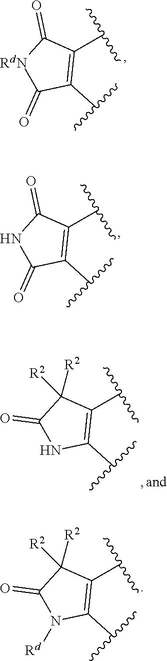

30. The compound of clause 27, wherein a pair of R¹ on adjacent atoms, taken together with the atoms connecting them, form a non-aromatic ring including 5 ring atoms, wherein from 1-2 ring atoms are heteroatoms each independently selected from the group consisting of N, N(H), N(R^{d}), O, and S(O)₀₋₂; wherein one ring atom is —O— or S(O)₀₋₂; and wherein the ring is optionally substituted with from 1-2 independently selected R² (e.g., tetrahydrofuranyl (e.g.,

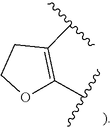 [SMILES: *C1=C(*)OCC1]

31. The compound of clause 19, wherein a pair of R¹ on adjacent atoms, taken together with the atoms connecting them, form a ring including 6 ring atoms (e.g., an aromatic ring including 6 ring atoms (e.g., pyridinyl or pyrimidinyl), wherein from 0-2 ring atoms are heteroatoms each independently selected from the group consisting of N, N(H), N(R^{d}), O, and S(O)₀₋₂; and wherein the ring is optionally substituted with from 1-4 independently selected R².

32. The compound of clause 31, wherein a pair of R¹ on adjacent atoms, taken together with the atoms connecting them, form pyridinyl (including pyridonyl), which is optionally substituted with from 1-3 independently selected R².

33. The compound of clause 32, wherein a pair of R¹ on adjacent atoms, taken together with the atoms connecting them, form:

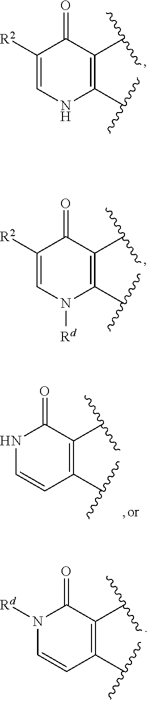

34. The compound of clause 19, wherein a pair of R¹ on adjacent atoms, taken together with the atoms connecting them, form a cycloalkyl ring including from 5-6 ring atoms; and wherein the ring is optionally substituted with from 1-4 independently selected R²,

35. The compound of clause 34, wherein a pair of R¹ on adjacent atoms, taken together with the atoms connecting them, form

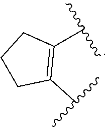 [SMILES: *C1=C(*)CCC1]

36. The compound of clause 17, wherein a pair of R¹ on adjacent atoms, taken together with the atoms connecting them, form a ring including from 7-12 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

37. The compound of clause 36, wherein a pair of R¹ on adjacent atoms, taken together with the atoms connecting them, form a ring including from 8-12 (e.g., 8; or e.g., 9-12 (e.g., 9, 10, 11, or 12)) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

38. The compound of clause 37, wherein a pair of R¹ on adjacent atoms, taken together with the atoms connecting them, form a spirocyclic bicyclic ring including from 8-12 (e.g., 9-12 (e.g., 9, 10, 11, or 12)) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

39. The compound of clause 38, wherein a pair of R¹ on adjacent atoms, taken together with the atoms connecting them, form:

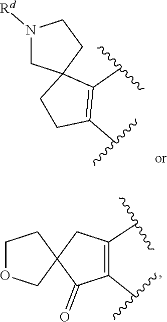

each of which is further optionally substituted with from 1-2 independently selected R².

40. The compound of any one of clauses 1-3 and 17-39, wherein the compound has the following formula:

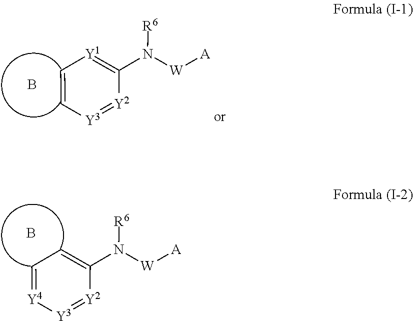

wherein ring B is a ring (e.g., monocyclic ring, bicyclic ring, or tricyclic ring) including from 4-15 (e.g., 5-12 (e.g., 5-10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

41. The compound of any one of clauses 1-3 and 40, wherein the compound has the following formula:

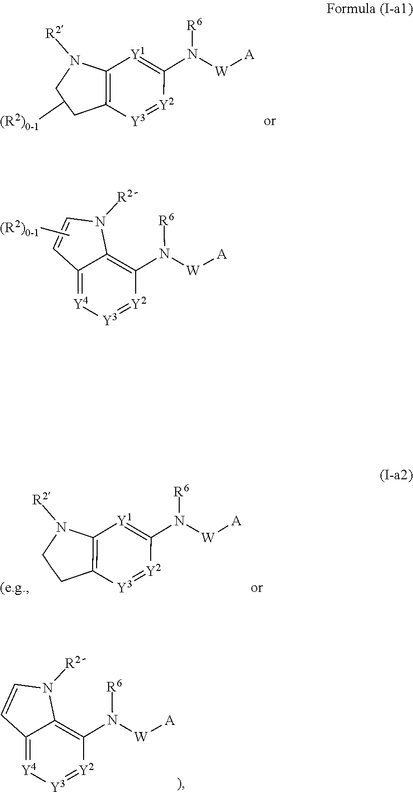

wherein R^{2′} is H or R² (e.g., R^{2′} is H) (in certain embodiments, the compound has Formula (I-a1); in certain of these embodiments, R^{2′} is H; in certain of these embodiments, Y³ is CR¹, wherein the R¹ is other than hydrogen; in certain of these embodiments, R² is present; in other embodiments, R² is absent).

42. The compound of any one of clauses 1-3 and 40, wherein the compound has the following formula:

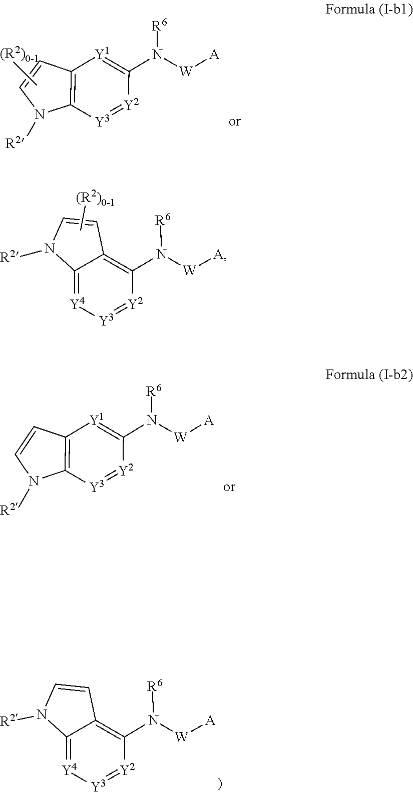

wherein R^{2′} is H or R² (e.g.,

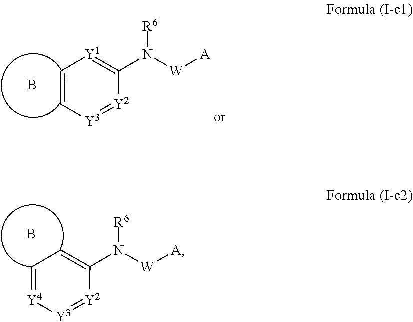

(e.g., R^{2′} is H) (in certain embodiments, the compound has Formula (I-b1); in certain of these embodiments, R^{2′} is H).

43. The compound of any one of clauses 1-3 and 40, wherein the compound has the following formula:

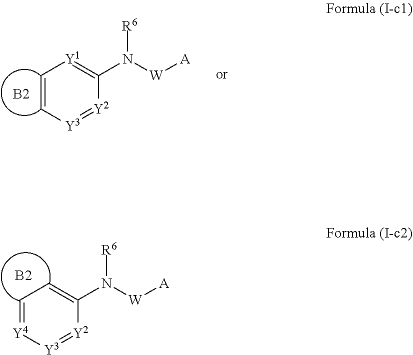

wherein B2 is an aromatic ring including 5 ring atoms, wherein from 1-2 (e.g., 2) ring atoms are heteroatoms each independently selected from the group consisting of N, N(H), N(R^{d}), O, and S(O)₀₋₂, provided that B2 is other than pyrrolyl; and wherein the ring is optionally substituted with from 1-4 independently selected R².

44. The compound of clause 43, wherein B2 is pyrazolyl, imidazolyl, or thiazolyl ring optionally substituted with from 1-2 independently selected R².

45. The compound of clause 43, wherein B2 is

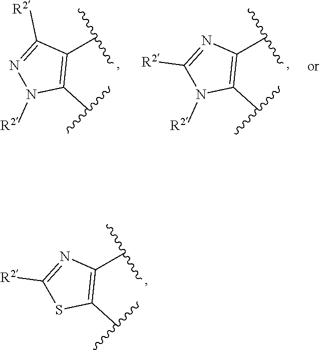

wherein each R^{2′} is independently H or R² (e.g.,

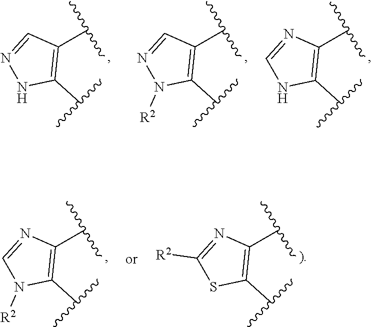

46. The compound of any one of clauses 1-3 and 40, wherein the compound has the following formula:

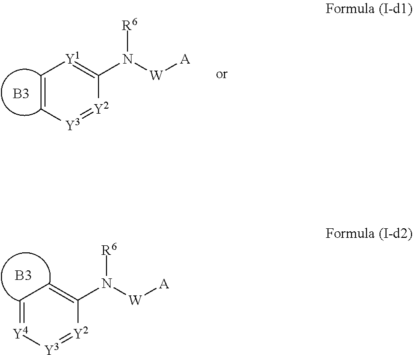

wherein B3 is selected from the group consisting of:

a) a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

b) a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

47. The compound of clause 46, wherein B3 is a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

48. The compound of clause 47, wherein B3 is a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

49. The compound of clause 48, wherein B3 is a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R² (e.g.,

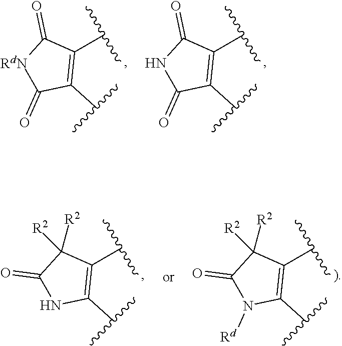

50. The compound of clause 48, wherein B3 is non-aromatic ring including 5 ring atoms, wherein from 0-1 ring atoms is a heteroatom selected from the group consisting of N, N(H), N(R^{d}), O, and S(O)₀₋₂; wherein the ring is optionally substituted with from 1-2 independently selected R² (e.g.,

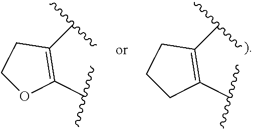 [SMILES: *C1=C(*)OCC1 ; *C1=C(*)CCC1]

51. The compound of clause 46, wherein B3 is a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R². 52. The compound of clause 51, wherein B3 is a spirocyclic bicyclic ring including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

53. The compound of clause 52, wherein B3 is

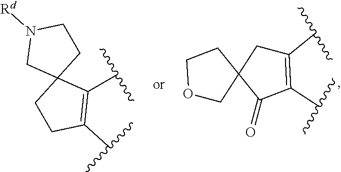

each of which is further optionally substituted with from 1-2 independently selected R².

54. The compound of any one of clauses 1-3 and 40, wherein the compound has the following formula:

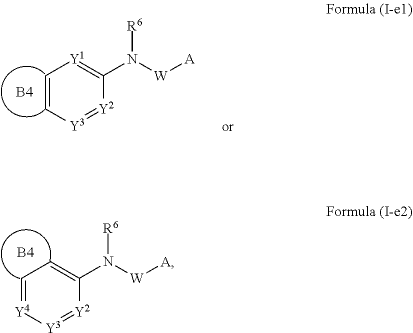

wherein B4 is an aromatic ring including 6 ring atoms, wherein from 0-2 ring atoms are heteroatoms each independently selected from the group consisting of N, N(H), and N(R^{d}); and wherein the ring is optionally substituted with from 1-4 independently selected R².

55. The compound of clause 54, wherein B4 is pyridinyl (including pyridonyl), which is optionally substituted with from 1-3 independently selected R² (e.g.,

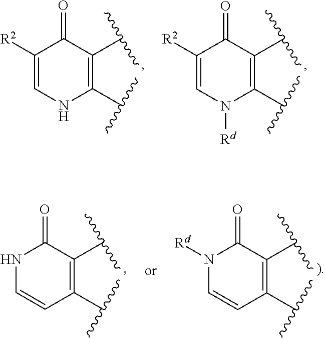

56. The compound of any one of clauses 40-55, wherein when the compound is of formula (I-1), (I-a1), (I-b1), (I-c1), (I-d1), or (I-e1), each of Y¹, Y², and Y³ is an independently selected CR¹; and

when the compound is of formula (I-2), (I-a2), (I-b2), (I-c2), (I-d2), or (I-e2), each of Y², Y³, and Y⁴ is an independently selected CR¹.

57. The compound of any one of clauses 40-55, wherein when the compound is of formula (I-1), (I-a1), (I-b1), (I-c1), (I-d1), or (I-e1), one of Y¹, Y², and Y³ is N; and each of the remaining of Y¹, Y², and Y³ is an independently selected CR¹; and

when the compound is of formula (I-2), (I-a2), (I-b2), (I-c2), (I-d2), or (I-e2), one of Y², Y³, and Y⁴ is N; and each of the remaining of Y², Y³, and Y⁴ is an independently selected CR¹.

58. The compound of any one of clauses 1, 40-41, and 56, wherein the compound has formula

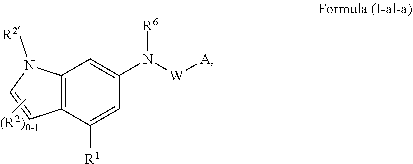

wherein R^{2′} is H or R².

59. The compound of clause 58, wherein the compound has Formula (I-a1-b):

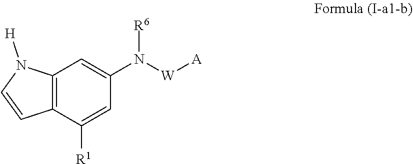

(e.g., R¹ is other than H).

60. The compound of clause 58, wherein the compound has Formula (I-a1-c):

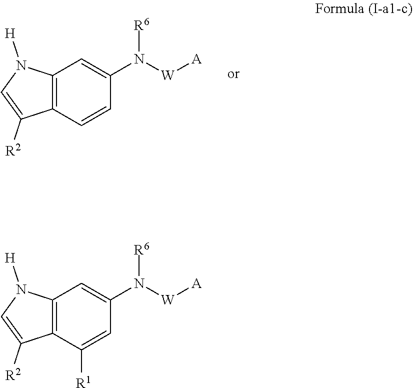

(e.g., R¹ is other than H) Formula (I-a1-e).

61. The compound of clause 58, wherein the compound has Formula (I-a1-d):

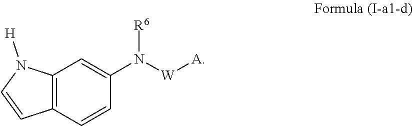

62. The compound of any one of clauses 1, 40, 42, and 56, wherein the compound has Formula (I-b1-a):

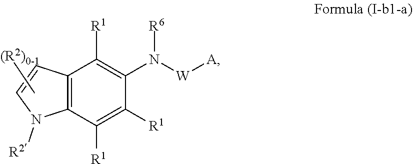

wherein R^{2′} is H or R².

63. The compound of clause 62, wherein the compound has Formula (I-b1-b):

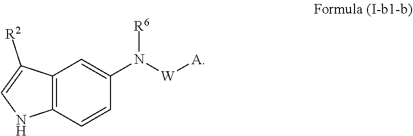

64. The compound of clause 62, wherein the compound has Formula (I-b1-c):

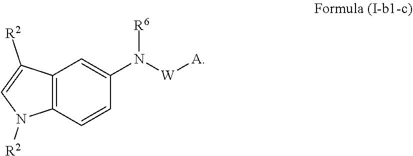

65. The compound of clause 62, wherein the compound has Formula (I-b1-d):

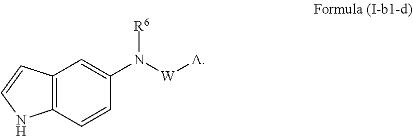

66. The compound of any one of clauses 1-65, wherein each occurrence of R¹ that is not taken together with the atom to which it is attached in ring formation is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —NR^{e}R^{f}; —OH; oxo; —S(O)₁₋₂(NR′R″); —C(═O)(C₁₋₄ alkyl); —C(═O)O(C₁₋₄ alkyl); —C(═O)OH; —C(═O)N(R′)(R″); and -L³-L⁴-Rⁱ.

67. The compound of any one of clauses 1-66, wherein each occurrence of R¹ that is not taken together with the atom to which it is attached in ring formation is H.

68. The compound of any one of clauses 1-66, wherein from 1-3 (e.g., 1, 2, or 3) occurrences of R¹ that is not taken together with the atom to which it is attached in ring formation is other than H; and each of the remaining occurrence of R¹ that is not taken together with the atom to which it is attached in ring formation is H.

69. The compound of clause 68, wherein one occurrence of R¹ that is not taken together with the atom to which it is attached in ring formation is other than H; and each of the remaining occurrence of R¹ that is not taken together with the atom to which it is attached in ring formation is H.

70. The compound of any one of clauses 1-66 and 68-69, wherein one occurrence of R¹ is halo (e.g., —F or —Cl ).

71. The compound of any one of clauses 1-66 and 68-69, wherein one occurrence of R¹ is NR^{e}R^{f} (e.g., NHAc) or C₁₋₄ alkoxy (e.g., methoxy).

72. The compound of any one of clauses 1-66 and 68-69, wherein one occurrence of R¹ is C₁₋₆ alkyl optionally substituted with 1-2 R^{a} (e.g., methyl, CH₂OH, or CH₂CH₂OH).

73. The compound of any one of clauses 1-66 and 68-69, wherein one occurrence of R¹ is cyano.

74. The compound of any one of clauses 1-66 and 68-69, wherein one occurrence of R¹ is selected from the group consisting of C(═O)OH and C(═O)O(C₁₋₄ alkyl).

75. The compound of clause 1-66 and 68-69, wherein one occurrence of R¹ is —L³-L⁴-Rⁱ (e.g., -L³ is a bond; and -L¹ is —O— (e.g., R¹ is phenoxy); or -L³ is a bond; and —L⁴ is a bond (e.g., R¹ is pyrazolyl or phenyl)).

76. The compound of any one of clauses 1-75, wherein each occurrence of R² is independently selected from the group consisting of: halo; cyano; C₁₋₆ alkyl optionally substituted with 1-2 R^{a}; C₁₋₄ haloalkyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; oxo; —S(O)₁₋₂(NR′R″); —C₁₋₄ thioalkoxy; —NO₂; —C(═O)(C₁₋₄ alkyl); —C(═O)O(C₁₋₄ alkyl); —C(═O)OH; —C(═O)N(R′)(R″); and -L³-L⁴-L⁵-Rⁱ.

77. The compound of any one of clauses 1-76, wherein one occurrence of R² is halo (e.g., F, Cl , or Br (e.g., F or Cl ) or cyano.

78. The compound of any one of clauses 1-76, wherein one occurrence of R² is C₁₋₆ alkyl optionally substituted with 1-2 R^{a}.

79. The compound of clause 78, wherein each occurrence of R^{a} is independently —F, —Cl , —OH, C₁₋₄ alkoxy, C₁₋₄ haloalkoxy, and —NR^{e}R^{f} (e.g., R² is methyl, CH₂OH, or CH₂CH₂OH).

80. The compound of any one of clauses 1-76, wherein one occurrence of R² is oxo; or wherein one occurrence of R² is OH.

81. The compound of any one of clauses 1-76, wherein one occurrence of R² is NR^{e}R^{f}.

82. The compound of clause 81, wherein each of R^{e} and R^{f} is independently selected from H; C₁₋₆ alkyl optionally substituted with from 1-2 substituents each independently selected from halo, OH, C₁₋₄ alkoxy, C₁₋₄ haloalkoxy, and CN; —C(O)(C₁₋₄ alkyl); —C(O)O(C₁₋₄ alkyl); —CON(R′)(R″); —S(O)₁₋₂(C₁₋₄ alkyl); and —S(O)(═NR′)(C₁₋₄ alkyl).

83. The compound of clause 82, wherein one of R^{e} and R^{f} is H (e.g., NR^{e}R^{f} is NHAc, NHS(O)₂Me, NHS(O)(═NH)Me, or NH(CH₂CH₂OH)).

84. The compound of any one of clauses 1-76, wherein one occurrence of R² is -L³-L⁴-L⁵-R¹.

85. The compound of clause 84, wherein -L³ of R² is a bond.

86. The compound of clause 84, wherein -L³ of R² is C₁₋₃ alkylene (e.g., CH₂).

87. The compound of any one of clauses 84-86, wherein -L¹ of R² is NR^{N} (e.g., NH).

88. The compound of any one of clauses 84-86, wherein -L¹ of R² is a bond.

89. The compound of any one of clauses 84-86, wherein -L¹ of R² is selected from the group consisting of a —NR^{N}C(O)—, —NR^{N}S(O)₀₋₂- or —NR^{N}S(═O)(═NR^{N}) (e.g., R^{N} is H).

90. The compound of any one of clauses 84-86, wherein -L¹ of R² is selected from the group consisting of NR^{N}S(═O)(═NR^{N})NR^{N}, —NR^{N}S(O)₁₋₂NR^{N}—, and —NR^{N}C(O)NR^{N}— (e.g., R^{N} is H).

91. The compound of any one of clauses 84-90, wherein -L⁵ is a bond.

92. The compound of any one of clauses 84-91, wherein -L⁵ is C₁₋₃ alkylene (e.g., —CH(CH₃)CH₂—).

93. The compound of any one of clauses 84-92, wherein R¹ of R² is C₃₋₈(e.g., C₆) cycloalkyl optionally substituted with from 1-4 (e.g., from 1-2) substituents independently selected from the group consisting of halo, C₁₋₄ alkyl, and C₁₋₄ haloalkyl (in certain embodiments, it is provided that when Rⁱ is C₃₋₆ cycloalkyl optionally substituted with from 1-4 substituents independently selected C₁₋₄ alkyl, -L¹ is a bond, or -L² is —O—, —N(H)—, or —S—).

94. The compound of any one of clauses 84-92, wherein R¹ of R² is C₆₋₁₀ (e.g., C₆) aryl, which is optionally substituted with from 1-4 (e.g., from 1-2) substituents independently selected from the group consisting of halo, C₁₋₄ alkyl, and C₁₋₄ haloalkyl.

95. The compound of any one of clauses 84-92, wherein R¹ of R² is heteroaryl including from 5-6 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl; and C₁₋₄ haloalkyl.

96. The compound of clause 84, wherein R² is selected from the group consisting of:

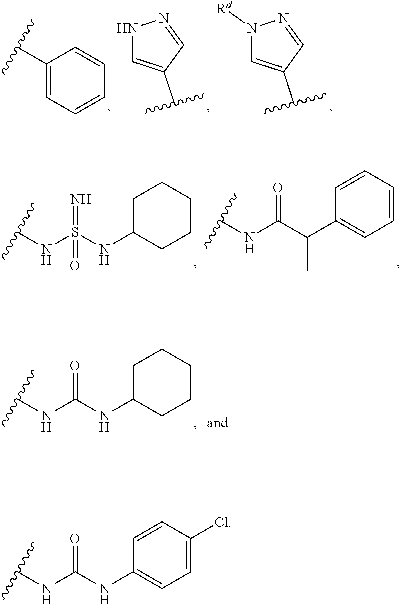

97. The compound of any one of clauses 1-76, wherein one occurrence of R² is C(O)OH.

98. The compound of any one of clauses 1-97, wherein W-A as defined according to (A).

99. The compound of any one of clauses 1-98, wherein W is selected from the group consisting of *C(═O)NR^{N}, *C(═S)NR^{N}, *C(═NR^{d})NR^{N} (e.g., *C(═NCN)NH), *C(═CNO₂)NR^{N}

100. The compound of clause 99, wherein W is *C(═O)NR^{N}.

101. The compound of clause 100, wherein W is *C(═O)NH or *C(═O)N(C₁₋₃ alkyl).

102. The compound of clause 101, wherein W is *C(═O)NH.

103. The compound of any one of clauses 1-98, wherein W is *S(O)₁₋₂NR^{N}.

104. The compound of clause 103, wherein W is *S(O)₂NR^{N} (e.g., *S(O)₂NH).

105. The compound of any one of clauses 1-98, wherein W is

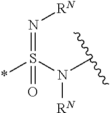

(e.g., each R^{N} is H).

106. The compound of any one of clauses 1-98, wherein W is

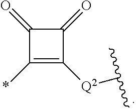

107. The compound of clause 106, wherein Q² is NR^{N}.

108. The compound of clause 107, wherein Q² is NH or N(C₁₋₃ alkyl).

109. The compound of clause 108, wherein Q² is NH.

110. The compound of any one of clauses 1-98, wherein W is -Q¹-Q²

111. The compound of clause 110, wherein -Q¹ is heteroarylene including from 5-6 ring atoms, wherein from 1-4 ring atoms are heteroatoms, each independently selected from the group consisting of N, N(H), N(R^{d}), O, and S, and wherein the heteroarylene ring is optionally substituted with from 1-4 independently selected R^{q1}.

112. The compound of clause 111, wherein Q¹ is heteroarylene including 6 ring atoms, wherein from 1-3 (e.g., 1-2) ring atoms are ring nitrogen atoms, and wherein the heteroarylene ring is optionally substituted with from 1-2 independently selected R^{q1}.

113. The compound of clause 112, wherein Q¹ is pyridylene or pyrimidinylene, each of which is optionally substituted with 1-2 independently selected R^{q1}.

114. The compound of clause 113, wherein Q¹ is selected from the group consisting of:

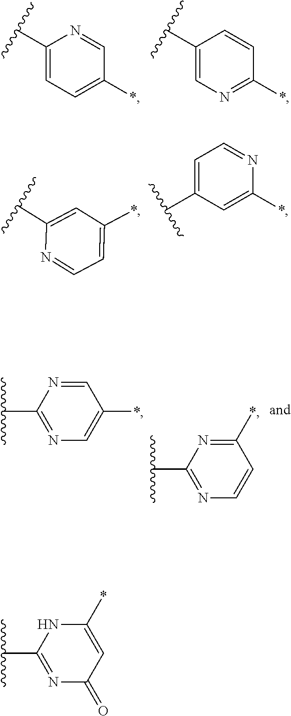

each of which is optionally substituted with 1-2 independently selected R^{q1}, wherein the asterisk denotes point of attachment of Q² (e.g.,

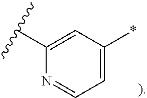 [SMILES: *c1ccnc(*)c1]

115. The compound of any one of clauses 110-114, wherein each R^{q1} is independently selected from the group consisting of: halo; cyano; C₁₋₁₀ alkyl which is optionally substituted with from 1-6 independently selected Rₐ (e.g., unsubstituted C₁₋₁₀ alkyl); C₃₋₆ cycloalkyl; and oxo.

116. The compound of any one of clauses 110-115, wherein Q² is a bond.

117. The compound of any one of clauses 110-115, wherein Q² is —O—, —NH—, or —S(O)₀₋₂(e.g., Q² is —O—; or Q² is —NH—; or Q² is —S(O)₂—).

118. The compound of any one of clauses 1-97, wherein W-A is as defined according to (B).

119. The compound of any one of clauses 1-97 and 118, wherein W is C₈₋₁₀ bicyclic arylene, which is optionally substituted with from 1-4 R^{c}.

120. The compound of any one of clauses 1-97 and 118, wherein W is bicyclic heteroarylene including from 8-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}.

121. The compound of clause 120, wherein W is heteroarylene including from 9-10 ring atoms, wherein from 1-3 (e.g., 1-2) ring atoms are heteroatoms, each independently selected from the group consisting of N, N(H), N(R^{d}), O, and S(O)₀₋₂, and wherein the heteroaryl ring is optionally substituted with from 1-4 (e.g., 1-2) independently selected R^{c}, such as a 10-membered heteroarylene selected from the group consisting of:

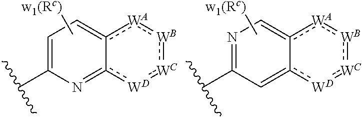

wherein w1 is 0 or 1;
each [symbol] is independently a single bond or a double bond;
any ring atom can serve as the point of attachment to A;

W^{A}, W^{B}, W^{C}, and W^{D} are each independently selected from the group consisting of: N, NH, NR^{d}, C, CH, CR^{c}, CH₂, CHR^{c}, C(R^{c})₂, provided that: no more than 2 of W^{A}, W^{B}, W^{C}, and W^{D} are N, NH, or NR^{d}; and W includes from 1-3 R^{c} (in certain embodiments, from 1-2 of W^{A}, W^{B}, and W^{C} is R^{c} (e.g., W^{C} is CR^{c})).

122. The compound of clause 121, wherein W is selected from the group consisting of quinolinylene, isoquinolinylene, and quinazolinylene, each of which is optionally substituted with from 1-2 independently selected R^{c}.

123. The compound of any one of clauses 120-122, wherein W is

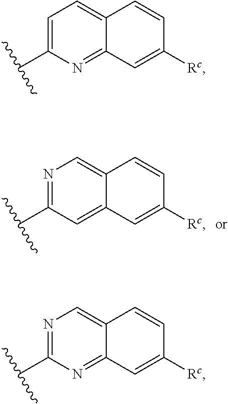

(e.g., R^{c} is C₁₋₃ alkyl, C₁₋₃ haloalkyl, or C₃₋₆ cycloalkyl).

124. The compound of any one of clauses 118-122, wherein A is H.

125. The compound of any one of clauses 1-117, wherein A is —Y^{A1}-Y^{A2}.

126. The compound of any one of clauses 1-117 and 125, wherein Y^{A1} is a bond.

127. The compound of any one of clauses 1-117 and 125, wherein Y^{A1} is C₁₋₆ alkylene, which is optionally substituted with from 1-4 R^{a}.

128. The compound of clause 127, wherein Y^{A1} is C₁₋₆ alkylene which is optionally substituted with from 1-2 R^{a}.

129. The compound of clause 128, wherein Y^{A1} is —CH₂—, —CH₂CH₂—, —CH₂CH₂CH₂—, —CH(CF₃)—, —CH₂CH(OH)—,

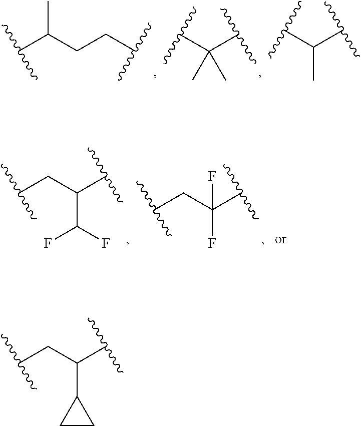

(e.g., Y^{A1} is CH₂).

130. The compound of clause 129, wherein Y^{A1} is —CH₂- or —CH₂CH₂-.

131. The compound of any one of clauses 1-117 and 125, wherein Y^{A1} is Y^{A3}-Y^{A4}—Y^{A5}.

132. The compound of clause 131, wherein Y^{A3} is C₂₋₃ alkylene.

133. The compound of any one of clauses 131-132, wherein Y^{A4} is —O— or -S-.

134. The compound of any one of clauses 131-133, wherein Y^{A5} is a bond.

135. The compound of clause 131, wherein Y^{A1} is

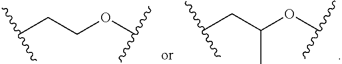

136. The compound of any one of clauses 131-133, wherein Y^{A5} is C₁₋₂ alkylene.

137. The compound of clause 131, wherein Y^{A1} is

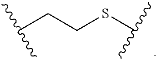 [SMILES: *CCS*]

138. The compound of any one of clauses 1-117 and 125-137, wherein Y^{A2} is C₆₋₁₀ aryl, which is optionally substituted with from 1-3 R^{c}.

139. The compound of any one of clauses 1-117 and 125-138, wherein Y^{A2} is C₆ aryl.

140. The compound of any one of clauses 1-117 and 125-139, wherein Y^{A2} is C₆ aryl, which is substituted with from 1-3 R^{c}.

141. The compound of any one of clauses 1-117 and 125-140, wherein Y^{A2} is phenyl substituted with from 1-3 (e.g., 1 or 2) R^{c}, wherein one R^{c} is at the ring carbon para to the point of attachment to Y^{A1}

142. The compound of any one of clauses 1-117 and 125-140, wherein Y^{A2} is phenyl substituted with from 1-3 (e.g., 1 or 2) R^{c}, wherein from 1-2 (e.g., 1) R^{c} is at the ring carbons meta to the point of attachment to Y^{A1}.

143. The compound of any one of clauses 1-117 and 125-140, wherein Y^{A2} is phenyl substituted with from 1-3 (e.g., 1 or 2) R^{c}, wherein from 1-2 (e.g., 1) R^{c} is at the ring carbons ortho to the point of attachment to Y^{A1}

144. The compound of any one of clauses 1-117 and 125-138, wherein Y^{A2} is C₇₋₁₀ bicyclic aryl, which is optionally substituted with from 1-3 R^{c} (e.g., Y^{A2} is naphthyl (e.g.,

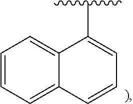 [SMILES: *c1cccc2ccccc12]

indenyl (e.g.,

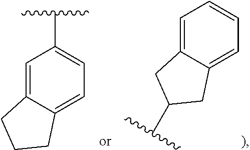 [SMILES: *c1ccc2c(c1)CCC2 ; *C1Cc2ccccc2C1]

or tetrahydronapthyl, each of which is optionally substituted with from 1-3 R^{c}).

145. The compound of any one of clauses 1-117 and 125-137, wherein Y^{A2} is heteroaryl including from 5-14 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}.

146. The compound of any one of clauses 1-117, 125-137, and 145, wherein Y^{A2} is heteroaryl including 5 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-3 independently selected R^{c}.

147. The compound of clause 146, wherein Y^{A2} is thiazolyl, thiadiazolyl, isoxazolyl triazolyl, or pyrazolyl, each of which is optionally substituted with from 1-2 (e.g., 1) independently selected R^{c} (e.g., Y^{A2} is pyrazolyl which is optionally substituted with from 1-2 (e.g., 1) independently selected R^{c} (e.g., Y^{A2} is

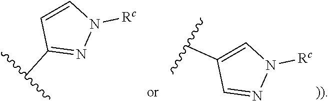

148. The compound of any one of clauses 1-117, 125-137, and 145, wherein Y^{A2} is heteroaryl including 6 ring atoms (e.g., pyridyl or pyrimidinyl (e.g., pyridyl (e.g.,

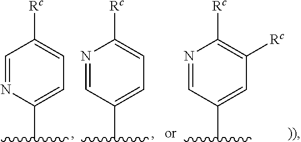

wherein from 1-2 ring nitrogen atoms, and wherein the heteroaryl ring is optionally substituted with from 1-3 independently selected R^{c}.

149. The compound of clause 148, wherein Y^{A2} is substituted with from 1-3 independently selected R^{c}; and one occurrence of R^{c} is at the ring carbon atom para to the point of attachment to Y^{A1}

150. The compound of clause 148, wherein Y^{A2} is substituted with from 1-3 independently selected R^{c}; and from 1-2 occurrences of R^{c} is at the ring carbon atom meta to the point of attachment to Y^{A1}

151. The compound of any one of clauses 1-117, 125-137, and 145, wherein Y^{A2} is bicyclic or tricyclic heteroaryl including from 7-14 (e.g., 9-12 (e.g., 9, 10, 11, or 12)) ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c} (e.g., Y^{A2} is

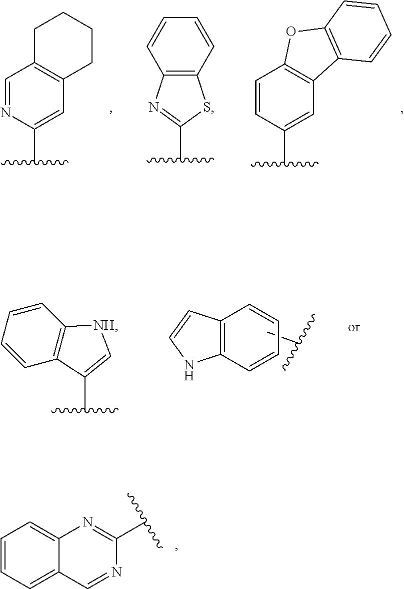

each of which is optionally substituted with from 1-2 independently selected R^{c}).

152. The compound of any one of clauses 1-151, wherein each occurrence of R^{c} is independently selected from the group consisting of: halo; cyano; C₁₋₁₀ alkyl which is optionally substituted with from 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)₁₋₂(C₁₋₄ haloalkyl); —NR^{e}R^{f}; —C₁₋₄ thioalkoxy; —C₁₋₄ thiohaloalkoxy; —SF₅; —C(═O)(C₁₋₁₀ alkyl); —C(═O)(OH); —C(═O)O(C₁₋₄ alkyl); and -L¹-L²-R^{h} (e.g., -R^{h}).

153. The compound of any one of clauses 1-152, wherein one occurrence of R^{c} is halo.

154. The compound of any one of clauses 1-152, wherein one occurrence of R^{c} is C₁₋₁₀ alkyl which is optionally substituted with from 1-6 independently selected R^{a}.

155. The compound of clause 154, wherein one occurrence of R^{c} is unsubstituted C₁₋₁₀ alkyl (e.g., C₂, C₃, C₄, C₅, C₆, or C₇₋₁₀), such as one occurrence of R^{c} is ethyl, propyl (e.g., n-propyl), butyl (e.g., n-butyl, iso-butyl, sec-butyl, tert-butyl), pentyl, or octyl (e.g., n-octyl) (e.g., R^{c} is butyl (e.g., n-butyl)).

156. The compound of clause 155, wherein one occurrence of R^{c} is unsubstituted C₆₋₁₀ alkyl (e.g., straight-chain C₆₋₁₀ alkyl).

157. The compound of clause 154, wherein one occurrence of R^{c} is C₁₋₁₀ alkyl which is substituted with from 1-6 independently selected R^{a}.

158. The compound of clause 157, wherein each occurrence of R^{a} is independently selected from —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl (e.g., each R^{a} is —F)).

159. The compound of any one of clauses 157-158, wherein one occurrence of R^{c} is selected from: CF₃, CHF₂, CH₂CF₃, CH₂CH₂CF₃, CH₂CH₂CH₂OH, CH₂CH₂OH, CH₂OH, CH₂CH₂OMe, CH₂OEt, CH₂OCH₂CH₂CH₃, CH(OH)CH₂CH₃, CH₂NMe₂, CH₂CH₂NMe₂, and

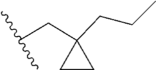 [SMILES: *CC1(CCC)CC1]

(e.g., R^{c} is CF₃).

160. The compound of any one of clauses 1-152, wherein one occurrence of R^{c} is C₂₋₆ alkenyl or C₂₋₆ alkynyl (e.g., C₂₋₆ alkynyl (e.g., acetylenyl)).

161. The compound of any one of clauses 1-152, wherein one occurrence of R^{c} is —C(═O)(C₁₋₁₀ alkyl) (e.g., —C(═O)(C3.1o alkyl) (e.g., —C(═O)CH₂CH₂CH₂CH₂CH₂CH₂CH₂)).

162. The compound of any one of clauses 1-152, wherein one occurrence of R^{c} is —SF₅.

163. The compound of any one of clauses 1-152, wherein one occurrence of R^{c} is —S(O)₁₋₂(NR′R″) (e.g.,

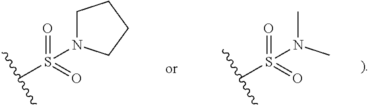

164. The compound of any one of clauses 1-152, wherein one occurrence of R^{c} is S(O)₁₋₂(C₁₋₄ alkyl) or S(O)₁₋₂(C₁₋₄ haloalkyl) (e.g., S(O)₂CF₃).

165. The compound of any one of clauses 1-152, wherein one occurrence of R^{c} is C₁₋₄ alkoxy or C₁₋₄ haloalkoxy (e.g., C₁₋₄ haloalkoxy such as OCF₃, OCF₂H, OCH₂CF₃, and OCH₂CF₂H).

166. The compound of any one of clauses 1-152, wherein one occurrence of R^{c} is -L¹-L²-R^{h} (e.g., R^{c} is -R^{h}).

167. The compound of clause 166, wherein L¹ is a bond.

168. The compound of clause 166, wherein L¹ is CH₂, CH₂CH₂, or C(═O).

169. The compound of any one of clauses 166-168, wherein L² is a bond.

170. The compound of any one of clauses 166-168, wherein L² is —O—.

171. The compound of clause 166, wherein L¹ is a bond; and L² is a bond.

172. The compound of clause 166, wherein L¹ is CH₂ or C(═O); and L² is a bond.

173. The compound of clause 166, wherein L¹ is a bond; and L² is —O—.

174. The compound of any one of clauses 166-173, wherein R^{h} is C₃₋₈ cycloalkyl optionally substituted with from 1-4 substituents independently selected from the group consisting of halo, C₁₋₄ alkyl, and C₁₋₄ haloalkyl.

175. The compound of clause 174, wherein R^{h} is C₃₋₆ cycloalkyl optionally substituted with from 1-4 substituents independently selected from the group consisting of halo, C₁₋₄ alkyl, and C₁₋₄ haloalkyl, such as

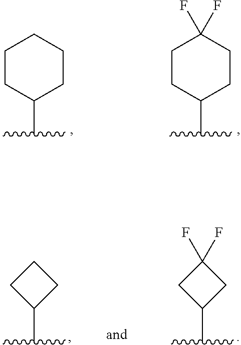 [SMILES: *C1CCCCC1 ; *C1CCC(F)(F)CC1 ; *C1CCC1 ; *C1CC(F)(F)C1]

176. The compound of any one of clauses 166-174, wherein R^{h} is C₆₋₁₀ aryl, which is optionally substituted with from 1-4 substituents independently selected from the group consisting of halo, cyano, C₁₋₄ alkyl, C₁₋₄ haloalkyl, C₁₋₄ alkoxy, and C₁₋₄ haloalkoxy, such as R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

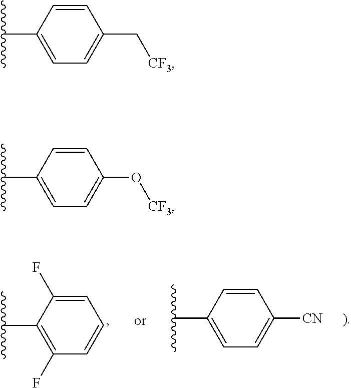 [SMILES: *c1ccc(CC(F)(F)F)cc1 ; *c1ccc(OC(F)(F)F)cc1 ; *c1c(F)cccc1F ; *c1ccc(C#N)cc1]

177. The compound of any one of clauses 166-173, wherein R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

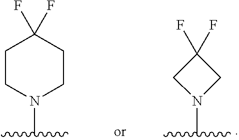 [SMILES: *N1CCC(F)(F)CC1 ; *N1CC(F)(F)C1]

178. The compound of any one of clauses 153-177, wherein each of the remaining R^{c} when present is independently halo or C₁₋₄ alkyl optionally substituted with R^{a}.

179. The compound of any one of clauses 1-117 and 125-137, wherein Y^{A2} is C₃₋₆ (e.g., C₃, C₅, or C₆) cycloalkyl, which is substituted with from 1-4 (e.g., from 1-2) R^{b} (e.g., Y^{A2} is cyclopropyl, cyclopentyl, bicyclo[1.1.1]pentyl, or cyclohexyl, each of which is optionally substituted with from 1-2 R^{b}).

180. The compound of clause 179, wherein Y^{A2} is cyclohexyl which is optionally substituted with from 1-2 R^{b}.

181. The compound of clause 180, wherein Y^{A2} is cyclohexyl which is optionally substituted with from 1-2 R^{b}, wherein one occurrence of R^{b} is at the ring carbon atom para to the point of attachment to Y^{A1}; or one occurrence of R^{b} is at the ring carbon atom meta to the point of attachment to Y^{A1}; or one occurrence of R^{b} is at the ring carbon atom ortho to the point of attachment to Y^{A1}.

182. The compound of clause 180, wherein Y^{A2} is cyclohexyl which is optionally substituted with from 1-2 R^{b} wherein one occurrence of R^{b} is at the ring carbon atom para to the point of attachment to Y^{A1}; or one occurrence of R^{b} is at the ring carbon atom meta to the point of attachment to Y^{A1} (e.g., one occurrence of R^{b} is at the ring carbon atom para to the point of attachment to Y^{A1}).

183. The compound of clause 179, wherein Y^{A2} is C₃₋₄ cycloalkyl which is optionally substituted with from 1-2 R^{b}.

184. The compound of any one of clauses 1-117 and 125-137, wherein Y^{A2} is C₇₋₁₀ cycloalkyl, which is optionally substituted with from 1-4 R^{b} (e.g., Y^{A2} is bicyclooctyl (e.g.,

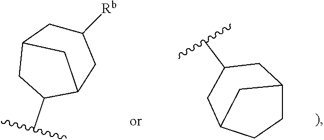

spirooctyl (e.g.,

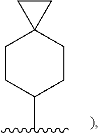 [SMILES: *C1CCC2(CC1)CC2]

or spiroundecanyl (e.g., spiro[5,5]undecanyl such as

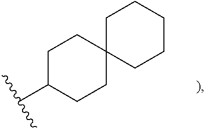 [SMILES: *C1CCC2(CCCCC2)CC1]

each of which is further optionally substituted with from 1-3 R^{b}).

185. The compound of any one of clauses 1-117 and 125-137, wherein Y^{A2} is heterocyclyl including from 3-12 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b}.

186. The compound of clause 185, wherein Y^{A2} is heterocyclyl including from 5-12 (e.g., 5-10) ring atoms, wherein from 1-3 (e.g., 1 or 2) ring atoms are heteroatoms, each independently selected from the group consisting of N, N(H), N(R^{d}), O, and S(O)₀₋₂, and wherein the heterocyclyl ring is optionally substituted with from 1-4 independently selected R^{b} (e.g., Y^{A2} is pyrrolidinyl (e.g.,

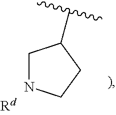 [SMILES: *C1CCNC1]

piperidinyl (e.g.,

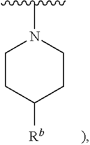

or tetrahydropyranyl (e.g.,

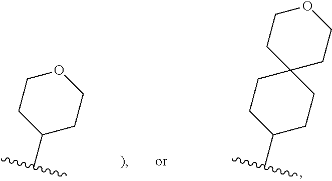 [SMILES: *C1CCOCC1 ; *C1CCC2(CCOCC2)CC1]

each of which is further optionally substituted with from 1-3 independently selected R^{b}).

187. The compound of clause 185, wherein Y^{A2} is heterocyclyl including from 5-6 (e.g., 5 or 6) ring atoms, wherein from 1-2 (e.g., 1 or 2) ring atoms are heteroatoms, each independently selected from the group consisting of N, N(H), N(R^{d}), O, and S(O)₀₋₂, and wherein the heterocyclyl ring is optionally substituted with from 1-4 independently selected R^{b} (e.g., Y^{A2} is pyrrolidinyl (e.g.,

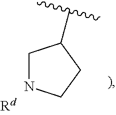 [SMILES: *C1CCNC1]

piperidinyl (e.g.,

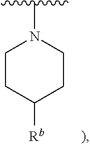

each of which is further optionally substituted with from 1-3 independently selected R^{b}).

188. The compound of clause 186, Y^{A2} is

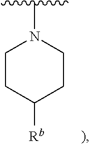

which is further optionally substituted with from 1-3 independently selected R^{b}.

189. The compound of any one of clauses 179-188, wherein each occurrence of R^{b} substituent of Y^{A2} is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

190. The compound of any one of clauses 179-189, wherein one occurrence of R^{b} substituent of Y^{A2} is C₁₋₁₀ alkyl which is optionally substituted with from 1-6 independently selected R^{a}.

191. The compound of clause 190, wherein one occurrence of R^{b} substituent of Y^{A2} is unsubstituted C₁₋₁₀ alkyl (e.g., C₂, C₃, C₄, C₅, C₆, or C₇₋₁₀).

192. The compound of clause 191, wherein one occurrence of R^{b} substituent of Y^{A2} is ethyl, propyl (e.g., n-propyl), butyl (e.g., n-butyl; or sec-butyl; or tert-butyl; or iso-butyl), or octyl (e.g., n-octyl) (e.g., butyl (e.g., n-butyl).

193. The compound of clause 190, wherein one occurrence of R^{b} substituent of Y^{A2} is C₁₋₁₀ alkyl which is substituted with from 1-6 independently selected R^{a}.

194. The compound of clause 193, wherein each occurrence of R^{a} is independently selected from —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

195. The compound of any one of clauses 179-189, wherein one occurrence of R^{b} is -L¹-L²-R^{h} (e.g., R^{b} is -R^{h}).

196. The compound of clause 195, wherein L¹ is a bond.

197. The compound of any one of clauses 195-196, wherein L² is a bond; or L² is —O—.

198. The compound of any one of clauses 195-197, wherein R^{h} is C₃₋₆ cycloalkyl optionally substituted with from 1-4 substituents independently selected from the group consisting of halo, C₁₋₄ alkyl, and C₁₋₄ haloalkyl.

199. The compound of any one of clauses 195-197, wherein R^{h} is heterocyclyl, wherein the heterocyclyl includes from 3-10 (e.g., 4, 5, 6, 7, 8, 9, or 10) ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo, C₁₋₄ alkyl, and C₁₋₄ haloalkyl, such as R^{h} is

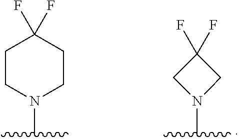 [SMILES: *N1CCC(F)(F)CC1 ; *N1CC(F)(F)C1]

200. The compound of any one of clauses 195-197, wherein R^{h} is C₆₋₁₀ aryl (e.g., C₆), which is optionally substituted with from 1-4 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

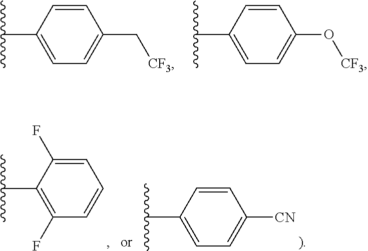 [SMILES: *c1ccc(CC(F)(F)F)cc1 ; *c1ccc(OC(F)(F)F)cc1 ; *c1c(F)cccc1F ; *c1ccc(C#N)cc1]

201. The compound of any one of clauses 179-189, wherein one occurrence of R^{b} is —Cl or —F (e.g., —F); or wherein one occurrence of R^{b} is oxo or cyano.

202. The compound of any one of clauses 190-201, wherein each remaining occurrence of R^{b} is independently selected from the group consisting of —Cl , —F, —Br, cyano, C₁₋₃ alkyl, and C₁₋₃ haloalkyl.

203. The compound of any one of clauses 1-117 and 125-137, wherein Y^{A2} is

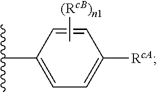

n1 is 0, 1, or 2; and each of R^{cA} and R^{cB} is an independently selected R^{c}.

204. The compound of any one of clauses 1-117 and 125-137, wherein Y^{A2} is

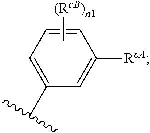

n1 is 0, 1, or 2; and each of R^{cA} and R^{cB} is an independently selected R^{c}.

205. The compound of any one of clauses 1-117 and 125-137, wherein Y^{A2} is

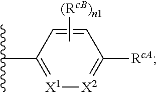

one of X¹ and X² is N; the other one of X¹ and X² is CH; n1 is 0, 1, or 2; and each of R^{cA} and R^{cB} is an independently selected R^{c}.

206. The compound of any one of clauses 1-117 and 125-137, wherein Y^{A2} is

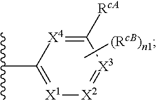

one of X¹, X², X³, and X⁴ is N; each of the remaining of X¹, X², X³, and X⁴ is CH; n1 is 0, 1, or 2; and each of R^{cA} and R^{cB} is an independently selected R^{c}.

207. The compound of any one of clauses 203-206, wherein R^{cA} is as defined for R^{c} in any one of clauses 153-165 (e.g., 153, 154, 155, 156, 157, 159, 160, 161, 162, 163, 164, or 165).

208. The compound of any one of clauses 203-206, wherein R^{cA} is C₁₋₁₀ alkyl which is substituted with from 1-6 independently selected R^{a}.

209. The compound of clause 208, wherein each R^{a} is independently selected from the group consisting —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl (e.g., each R^{a} is —F)).

210. The compound of any one of clauses 208-209, wherein R^{cA} is C₁₋₃ alkyl which is substituted with from 1-3 —F (e.g., R^{cA} is —CF₃).

211. The compound of clause 208, wherein R^{c} is unsubstituted C₁₋₁₀ alkyl (e.g., straight chain C₂, C₃, C₄, C₅, C₆, or C₇₋₁₀ alkyl).

212. The compound of any one of clauses 203-206, wherein R^{cA} is C₂₋₆ alkenyl; C₂₋₆ alkynyl; or -C(═O)(C₁₋₁₀ alkyl) (e.g., —C(═O)(C₃₋₁₀ alkyl) (e.g., —C(═O)CH₂CH₂CH₂CH₂CH₂CH₂CH₂)).

213. The compound of any one of clauses 203-206, wherein R^{cA} is selected from the group consisting of —SF₅; —S(O)₁₋₂(NR′R″) (e.g.,

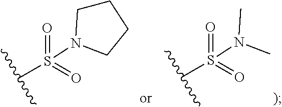

S(O)₁₋₂(C₁₋₄ alkyl); and S(O)₁₋₂(C₁₋₄ haloalkyl) (e.g., S(O)₂CF₃).

214. The compound of any one of clauses 203-206, wherein R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

215. The compound of any one of clauses 203-206, wherein R^{cA} is as defined for R^{c} in any one of clauses 166-177 (e.g. R^{c} is -L¹-L²-R^{h}, such as R^{h}; and R^{h} is as defined in clause 175, clause 176, or clause 177).

216. The compound of any one of clauses 203-206, wherein R^{cA} is -L¹-L²-R^{h}, wherein:

-L¹ is a bond, CH₂, or —CH₂CH₂;

-L² is a bond or —O— (e.g., -L¹ is a bond; and -L² is a bond); and

R^{h} is C₃₋₆ cycloalkyl optionally substituted with from 1-4 substituents independently selected from the group consisting of halo, C₁₋₄ alkyl, and C₁₋₄ haloalkyl (e.g.,

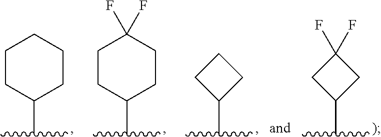

or

R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

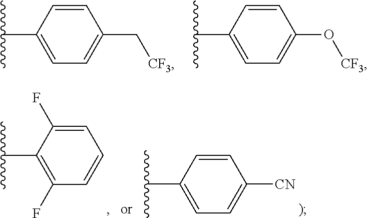

R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as

### R^{h} is

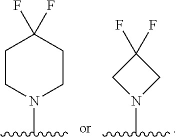 [SMILES: *N1CCC(F)(F)CC1 ; *N1CC(F)(F)C1]

217. The compound of any one of clauses 203-216, wherein n1 is 0.

218. The compound of any one of clauses 203-216, wherein n1 is 1 or 2.

219. The compound of clause 218, wherein each R^{cB} is independently halo or C₁₋₄ alkyl optionally substituted with R^{a}.

220. The compound of any one of clauses 1-117 and 125-137, wherein Y^{A2} is

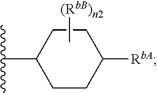

n2 is 0, 1, or 2; and each of R^{bA} and R^{bB} is an independently selected R^{b}.

221. The compound of any one of clauses 1-117 and 125-137, wherein Y^{A2} is

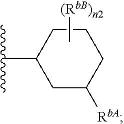

n2 is 0, 1, or 2; and each of R^{bA} and R^{bB} is an independently selected R^{b}.

222. The compound of any one of clauses 220-221, wherein R^{bA} is selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

223. The compound of any one of clauses 220-221, wherein R^{bA} is as defined for R^{b} in any one of clauses 190-194 (e.g., 190, 191, 192, 193, or 194).

224. The compound of clause 223, wherein R^{bA} is C₁₋₁₀ alkyl which is optionally substituted with from 1-6 independently selected R^{a}.

225. The compound of clause 224, wherein R^{bA} is unsubstituted C₁₋₁₀ alkyl (e.g., straight-chain C₂, C₃, C₄, C₅, C₆, or C₇₋₁₀ alkyl).

226. The compound of clause 223, wherein, R^{bA} is C₁₋₁₀ alkyl which is substituted with from 1-6 independently selected R^{a}, such as C₁₋₁₀ alkyl which is substituted with from 1-6 substituents each independently selected from the group consisting of: —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

227. The compound of any one of clauses 220-221, wherein R^{bA} is as defined for R^{b} in any one of clauses 195-200 (e.g., 195, 196, 197, 198, 199, or 200).

228. The compound of clause 227, wherein R^{bA} is -L¹-L²-R^{h}, wherein:

L¹ is a bond; L² is a bond or —O— (e.g., L¹ is a bond; and L² is a bond); and

R^{h} is C₃₋₆ cycloalkyl optionally substituted with from 1-4 substituents independently selected from the group consisting of halo, C₁₋₄ alkyl, and C₁₋₄ haloalkyl; or

R^{h} is C₆₋₁₀ aryl (e.g., C₆), which is optionally substituted with from 1-4 substituents independently selected from the group consisting of halo, C₁₋₆ alkyl, or C₁₋₄ haloalkyl (e.g., R^{h} is unsubstituted phenyl; or R^{h} is

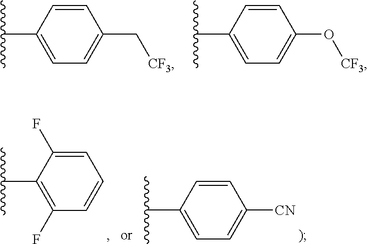 [SMILES: *c1ccc(CC(F)(F)F)cc1 ; *c1ccc(OC(F)(F)F)cc1 ; *c1c(F)cccc1F ; *c1ccc(C#N)cc1]

or

R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

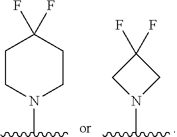 [SMILES: *N1CCC(F)(F)CC1 ; *N1CC(F)(F)C1]

229. The compound of anyone of clauses 220-221, wherein R^{bA} is —Cl or —F (e.g., F).

230. The compound of any one of clauses 220-229, wherein n2 is 0.

231. The compound of any one of clauses 220-229, wherein n2 is 1 or 2.

232. The compound of clause 231, wherein each R^{bB} is independently selected from the group consisting of —Cl , —F, C₁₋₃ alkyl, and C₁₋₃ haloalkyl.

233. The compound of any one of clauses 1-117, wherein A is C₁₋₂₀ alkyl, which is optionally substituted with from 1-6 independently selected R^{a}.

234. The compound of any one of clauses 1-117 and 233, wherein A is C₂₋₁₀ (e.g., C₂, C₃, C₄, C₅, C₆, C₇, C₈, C₉, C₁₀) alkyl, which is optionally substituted with from 1-6 independently selected R^{a}.

235. The compound of any one of clauses 1-117 and 233, wherein A is C₁₀₋₂₀ alkyl, which is optionally substituted with from 1-6 independently selected R^{a}.

236. The compound of clause 235, wherein A is unsubstituted C₁₀₋₂₀ alkyl (e.g., C₁₀₋₁₂, C₁₃₋₁₅, C₁₆₋₁₈, C₁₉₋₂₀ alkyl).

237. The compound of clause 236, wherein A is unsubstituted straight-chain C₁₀₋₂₀ alkyl (e.g., straight-chain C₁₀₋₁₂, C₁₃₋₁₅, C₁₆₋₁₈, C₁₉₋₂₀ alkyl).

238. The compound of clause 1, wherein the compound has the following formula:

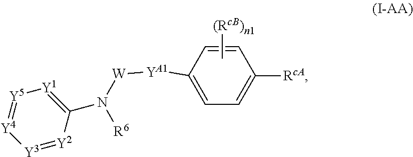

wherein n1 is 0, 1, or 2; and each of R^{cA} and R^{cB} is an independently selected R^{c}.

239. The compound of clause 1, wherein the compound has the following formula:

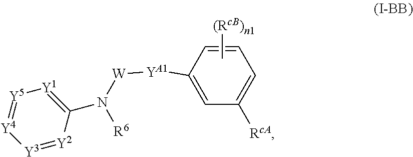

wherein n1 is 0, 1, or 2; and each of R^{cA} and R^{cB} is an independently selected R^{c}.

240. The compound of clause 1, wherein the compound has the following formula:

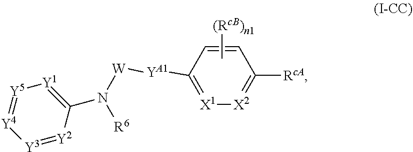

wherein one of X¹ and X² is N; the other one of X¹ and X² is CH; n1 is 0, 1, or 2; and each of R^{cA} and R^{cB} is an independently selected R^{c}.

241. The compound of clause 1, wherein the compound has the following formula:

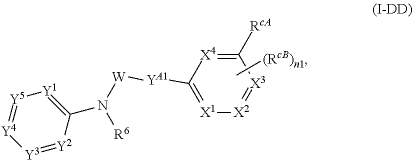

wherein one of X¹, X², X³, and X⁴ is N; each of the remaining of X¹, X², X³, X⁴ is CH; n1 is 0, 1, or 2; and each of R^{cA} and R^{cB} is an independently selected R^{c}.

242. The compound of clause 1, wherein the compound has the following formula:

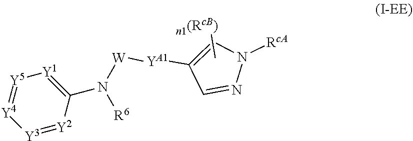

(e.g., R^{cA} is L¹-L₂-R^{h}),

wherein n1 is 0 or 1; and each of R^{cA} and R^{cB} is an independently selected R^{c}.

243. The compound of any one of clauses 238-242, wherein R^{cA} is as defined for R^{c} in any one of clauses 153-165 (e.g., 153, 154, 155, 156, 157, 159, 160, 161, 162, 163, 164, or 165); or wherein R^{cA} is as defined for R^{c} in any one of clauses 166-177 (e.g. R^{c} is -L¹-L²-R^{h}, such as R^{h}; and R^{h} is as defined in clause 175, clause 176, or clause 177).

244. The compound of any one of clauses 238-242, wherein R^{cA} is C₁₋₃ alkyl which is substituted with from 1-3 —F (e.g., R^{cA} is —CF₃); or

R^{cA} is unsubstituted C₁₋₁₀ alkyl (e.g., straight chain C₂, C₃, C₄, C₅, C₆, or C₇₋₁₀ alkyl); or R^{cA} is C₂₋₆ alkenyl, C₂₋₆ alkynyl, or —C(═O)(C₁₋₁₀ alkyl) (e.g., —C(═O)(C₃₋₁₀ alkyl) (e.g., —C(═O)CH₂CH₂CH₂CH₂CH₂CH₂CH₂));

R^{cA} is selected from the group consisting of —SF₅, —S(O)₁₋₂(NR′R″) (e.g.,

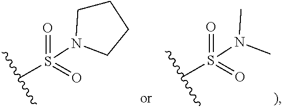

S(O)₁₋₂(C₁₋₄ alkyl), and S(O)₁₋₂(C₁₋₄ haloalkyl) (e.g., S(O)₂CF₃); or

R^{cA} is C₁₋₄ alkoxy or C₁₋₄ haloalkoxy (e.g., C₁₋₄ haloalkoxy such as OCF₃, OCF₂H, OCH₂CF₃, and OCH₂CF₂H).

245. The compound of any one of clauses 238-242, wherein R^{cA} is -L¹-L²-R^{h}, wherein:

-L¹ is a bond, CH₂, or —CH₂CH₂;

-L² is a bond or —O— (e.g., -L¹ is a bond; and -L² is a bond); and

R^{h} is C₃₋₆ cycloalkyl optionally substituted with from 1-4 substituents independently selected from the group consisting of halo, C₁₋₄ alkyl, and C₁₋₄ haloalkyl (e.g.,

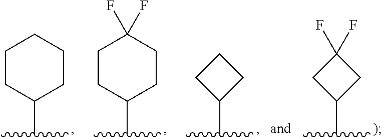

or

R^{h} is C₆ aryl, which is optionally substituted with from 1-2 substituents independently selected from the group consisting of halo, cyano, C₁₋₄ alkyl, C₁₋₄ haloalkyl, C₁₋₄ alkoxy, and C₁₋₄ haloalkoxy (e.g., R^{h} is unsubstituted phenyl; or R^{h} is

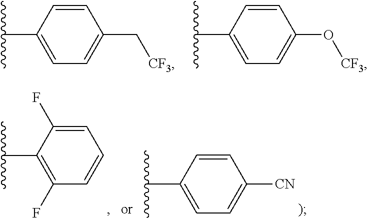

R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

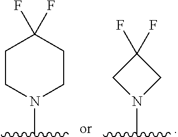 [SMILES: *N1CCC(F)(F)CC1 ; *N1CC(F)(F)C1]

246. The compound of any one of clauses 238-245, wherein n1 is 0.

247. The compound of any one of clauses 238-245, wherein n1 is 1.

248. The compound of any one of clauses 238-245 and 247, wherein each R^{cB} is independently halo or C₁₋₄ alkyl optionally substituted with R^{a}.

249. The compound of clause 1, wherein the compound has the following formula:

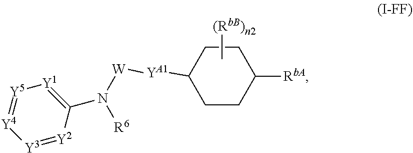

wherein n2 is 0, 1, or 2; and each of R^{bA} and R^{bB} is an independently selected R^{b}.

250. The compound of clause 1, wherein the compound has the following formula:

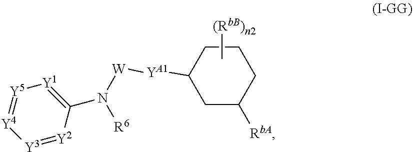

wherein n2 is 0, 1, or 2; and each of R^{bA} and R^{bB} is an independently selected R^{b}.

251. The compound of clause 1, wherein the compound has the following formula:

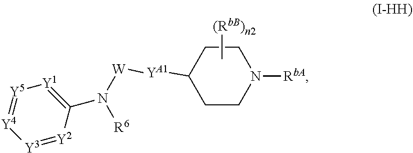

wherein n2 is 0, 1, or 2; and each of R^{bA} and R^{bB} is an independently selected R^{b}.

252. The compound of clause 1, wherein the compound has the following formula:

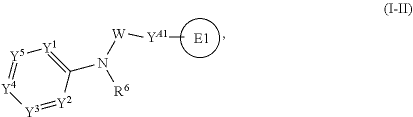

wherein ring E1 is C₇₋₁₀ cycloalkyl, which is optionally substituted with from 1-4 R^{b} (e.g., Y^{A2} is bicyclooctyl (e.g.,

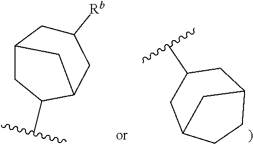

or spiroundecanyl (e.g., spiro[5,5]undecanyl such as

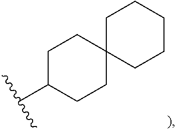 [SMILES: *C1CCC2(CCCCC2)CC1]

each of which is further optionally substituted with from 1-3 R^{b}).

253. The compound of any one of clauses 249-251, wherein R^{bA} is as defined in any one of clauses 190-194 (e.g., clause 190, 191, 192, 193, or 194).

254. The compound of any one of clauses 249-251 and 253, wherein R^{bA} is unsubstituted C₁₋₁₀ alkyl (e.g., straight-chain C₂, C₃, C₄, C₅, C₆, or C₇₋₁₀ alkyl); or R^{bA} is C₁₋₁₀ alkyl which is substituted with from 1-6 independently selected R^{a}, such as C₁₋₁₀ alkyl which is substituted with from 1-6 substituents each independently selected from the group consisting of: —F, —Cl , OH, C₁₋₄ alkoxy, NR^{e}R^{f}, C₁₋₄ haloalkoxy, and C₃₋₆ cycloalkyl optionally substituted with from 1-4 independently selected C₁₋₄ alkyl.

255. The compound of any one of clauses 249-251, wherein R^{bA} is as defined in any one of clauses 195-200 (e.g., clause 195, 196, 197, 198, 199, or 200).

256. The compound of any one of clauses 249-251 and 255, wherein R^{bA} is -L¹-L²-R^{h}, wherein:

L¹ is a bond; L² is a bond or —O—; and

R^{h} is C₃₋₆ cycloalkyl optionally substituted with from 1-4 substituents independently selected from the group consisting of halo, C₁₋₄ alkyl, and C₁₋₄ haloalkyl; or

R^{h} is C₆₋₁₀ aryl (e.g., C₆), which is optionally substituted with from 1-4 substituents independently selected from the group consisting of halo, C₁₋₄ alkyl, or C₁₋₄ haloalkyl (e.g., R^{h} is unsubstituted phenyl; or R^{h} is

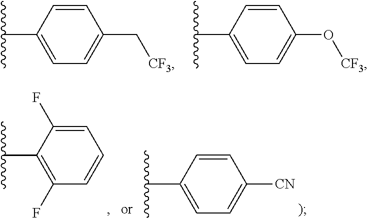

R^{h} is heterocyclyl, wherein the heterocyclyl includes from 4-10 (e.g., 4, 5, or 6) ring atoms, wherein from 1-3 (e.g., from 1-2; e.g., 1) ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy, such as R^{h} is

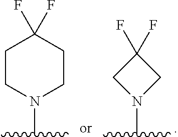 [SMILES: *N1CCC(F)(F)CC1 ; *N1CC(F)(F)C1]

257. The compound of anyone of clauses 249-251, wherein R^{bA} is —Cl or —F (e.g., —F).

258. The compound of any one of clauses 249-257, wherein n2 is 0.

259. The compound of any one of clauses 249-257, wherein n2 is 1 or 2.

260. The compound of any one of clauses 249-257 and 259, wherein each R^{bB} is independently —F, —Cl , or C₁₋₃ alkyl.

261. The compound of clause 252, wherein each occurrence of R^{b} is independently selected from the group consisting of: C₁₋₁₀ alkyl optionally substituted with from 1-6 independently selected R^{a}; C₁₋₄ haloalkyl; —F; —Cl ; —Br; cyano; C₁₋₄ alkoxy; C₁₋₄ haloalkoxy; —C(═O)(C₁₋₁₀ alkyl); —C(═O)O(C₁₋₄ alkyl); —S(O)₁₋₂(C₁₋₄ alkyl); oxo; cyano; and -L¹-L²-R^{h}.

262. The compound of any one of clauses 238-261, wherein Y^{A1} is a bond.

263. The compound of any one of clauses 238-261, wherein Y^{A1} is CH₂ or C(═O).

264. The compound of any one of clauses 238-261, wherein Y^{A1} is —CH₂—, —CH₂CH₂—, —CH₂CH₂CH₂—, —CH(CF₃)—, —CH₂CH(OH)—,

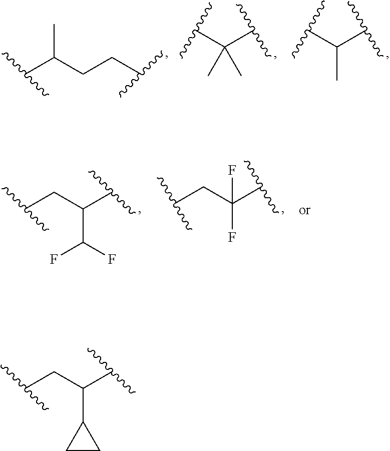

(e.g., Y^{A1} is CH₂).

265. The compound of clause 1, wherein the compound has the following formula:

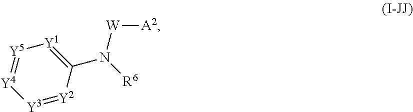

wherein A² is C₁₋₂₀ alkyl, which is optionally substituted with from 1-6 independently selected R^{a}.

266. The compound of clause 265, wherein A² is C₈₋₂₀ (e.g., C₈, C9, C₁₀, C₁₁₋₁₃, C₁₄₋₁₆, C₁₇₋₁₉, or C₂₀) alkyl, which is optionally substituted with from 1-6 independently selected R^{a}.

267. The compound of any one of clauses 265-266, wherein A² is unsubstituted C₈₋₂₀ (e.g., C₈, C₉, C₁₀, C₁₁₋₁₃, C₁₄₋₁₆, C₁₇₋₁₉, or C₂₀) alkyl.

268. The compound of clause 266, wherein A² is unsubstituted C₁₀₋₂₀ (e.g., C₁₀, C₁₁₋₁₃, C₁₄₋₁₆, C₁₇₋₁₉, or C₂₀) alkyl.

269. The compound of clause 266, wherein A² is straight-chain C₁₀₋₂₀ (e.g., C₁₀, C₁₁₋₁₃, C₁₄₋₁₆, C₁₇₋₁₉, or C₂₀) alkyl.

270. The compound of any one of clauses 238-269, wherein W is *C(═)NR^{N}.

271. The compound of clause 270, wherein W is *C(═O)NH or *C(═O)N(C₁₋₃ alkyl).

272. The compound of clause 271, wherein W is *C(═O)NH.

273. The compound of any one of clauses 238-269, wherein W is *S(O)₁₋₂NR^{N}.

274. The compound of clause 273, wherein W is *S(O)₂NR^{N} (e.g., *S(O)₂NH).

275. The compound of any one of clauses 238-269, wherein W is *C(═NR^{N})NR^{N} (e.g., C(═NCN)NH).

276. The compound of any one of clauses 238-269, wherein W is

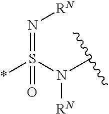

each R^{N} is H).

277. The compound of any one of clauses 238-269, wherein W is

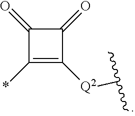

278. The compound of clause 277, wherein Q² is NR^{N}.

279. The compound of clause 278, wherein Q² is NH or N(C₁₋₃ alkyl) (e.g., NH).

280. The compound of any one of clauses 238-269, wherein W is -Q¹-W² (e.g., —Q¹ is heteroarylene including 6 ring atoms, wherein from 1-3 (e.g., 1-2) ring atoms are ring nitrogen atoms, and wherein the heteroarylene ring is optionally substituted with from 1-2 independently selected R^{q1}).

281. The compound of clause 280, wherein Q¹ is selected from the group consisting of:

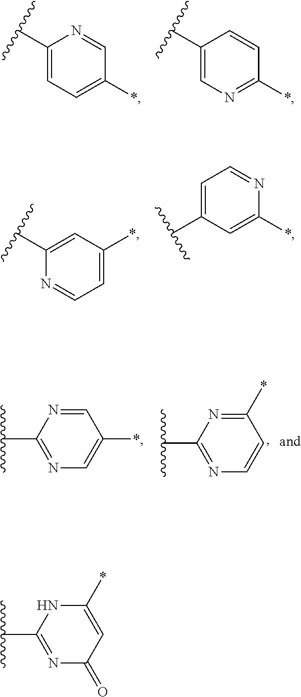

and each of which is optionally substituted with 1-2 independently selected R^{q1}, wherein the asterisk denotes point of attachment of Q² (e.g.,

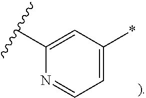 [SMILES: *c1ccnc(*)c1]

282. The compound of any one of clauses 280-281, wherein Q² is a bond.

283. The compound of any one of clauses 280-281, wherein Q² is —O—, —NH—, or —S(O)₀₋₂(e.g., Q² is —O—; or Q² is —NH—; or Q² is —S(O)₂—).

284. The compound of clause 1, wherein the compound has Formula (I-KK):

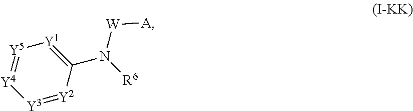

wherein A is H; and

W is selected from the group consisting of:

C₈₋₁₀ bicyclic arylene, which is optionally substituted with from 1-4 R^{c}; and

heteroarylene including from 8-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-3 independently selected R^{c}.

285. The compound of clause 284, wherein W is heteroarylene including from 9-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-2 independently selected R^{c}, such as a 10-membered heteroarylene selected from the group consisting of:

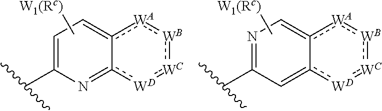

wherein w1 is 0 or 1;
each [symbol] is independently a single bond or a double bond;
any ring atom can serve as the point of attachment to A;

W^{A}, W^{B}, W^{C}, and W^{D} are each independently selected from the group consisting of: N, NH, NR^{d}, C, CH, CR^{c}, CH₂, CHR^{c}, C(R^{c})₂, provided that: no more than 2 of W^{A}, W^{B}, W^{C}, and W^{D} are N, NH, or NR^{d}; and W includes from 1-3 R^{c} (in certain embodiments, from 1-2 of W^{A}, W^{B}, and W^{c} is R^{c} (e.g., W^{C} CR^{c})).

286. The compound of any one of clauses 284-285, wherein W is selected from the group consisting of quinolinylene and quinazolinylene, each of which is optionally substituted with from 1-2 independently selected R^{c}.

287. The compound of clause 286, wherein W is

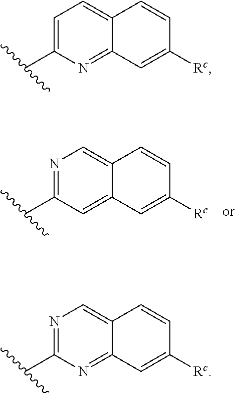

288. The compound of any one of clauses 284-287, wherein one occurrence of R^{c} is C₁₋₁₀ alkyl which is substituted with from 1-6 independently selected R^{a} (e.g., —CF₃).

289. The compound of any one of clauses 284-287, wherein one occurrence of R^{c} is halo (e.g., —Cl or F).

290. The compound of any one of clauses 284-287, wherein one occurrence of R^{c} is -L¹-L²-R^{h}.

291. The compound of clause 290, wherein one occurrence R^{c} is R^{h}, wherein R^{h} is C₃₋₆ cycloalkyl optionally substituted with from 1-4 substituents independently selected from the group consisting of halo, C₁₋₄ alkyl, and C₁₋₄ haloalkyl (e.g.,

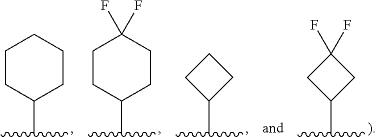

292. The compound of any one of clauses 238-291, wherein the

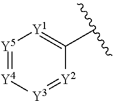

moiety is

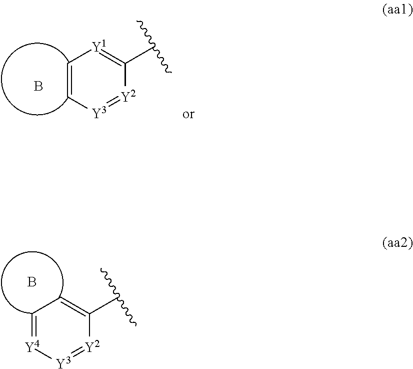

wherein ring B is a ring (e.g., monocyclic ring, bicyclic ring, or tricyclic ring) including from 4-15 (e.g., 5-12 (e.g., 5-10)) ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R².

293. The compound of any one of clauses 238-292, wherein the

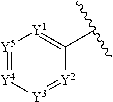

moiety is

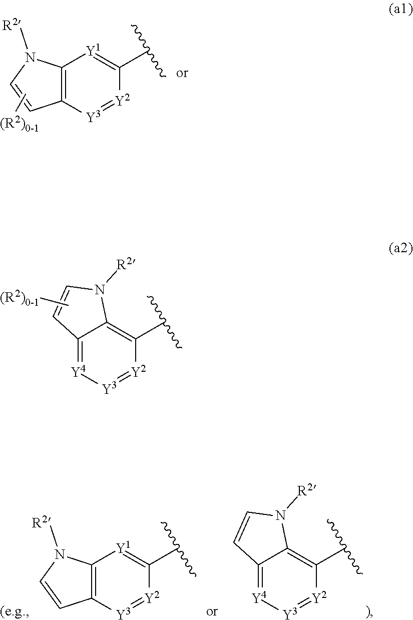

wherein R^{2′} is H or R² (e.g., R^{2′} is H).

294. The compound of any one of clauses 238-292, wherein the

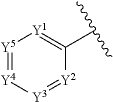

moiety is

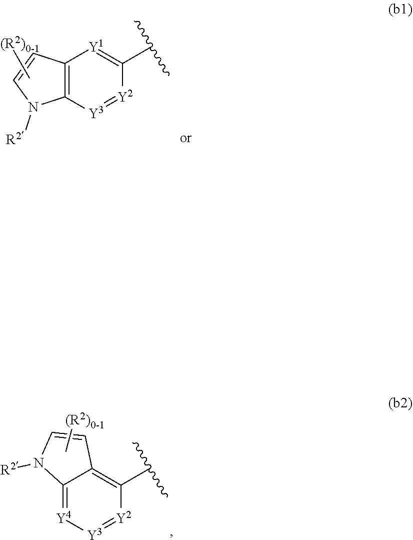

wherein R^{2′} is H or R² (e.g.,

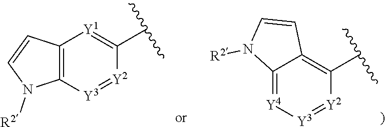

### (e.g., R^{2′} is H).

295. The compound of any one of clauses 238-292, wherein the

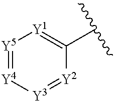

moiety is

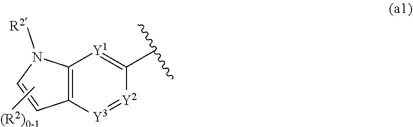

(e.g.,

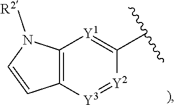

wherein R^{2′} is H or R² (e.g., R^{2′} is H).

296. The compound of clause 295, wherein the

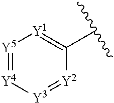

moiety is

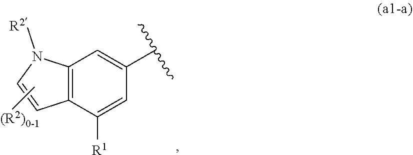

wherein R^{2′} is H or R².

297. The compound of clause 295, wherein the

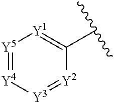

moiety is

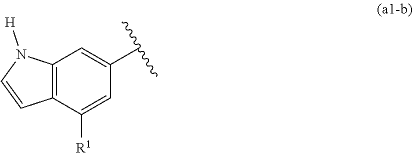

(e.g., R¹ is other than H (e.g., R¹ is halo or cyano)).

298. The compound of clause 295, wherein the

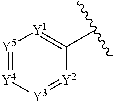

moiety is

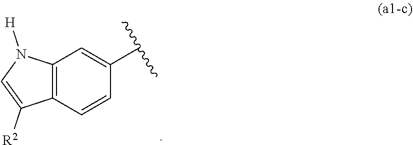

299. The compound of clause 295, wherein the

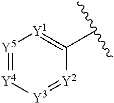

moiety is

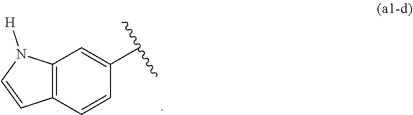 [SMILES: *c1ccc2cc[nH]c2c1]

300. The compound of any one of clauses 238-292, wherein the

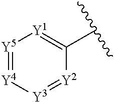

moiety is

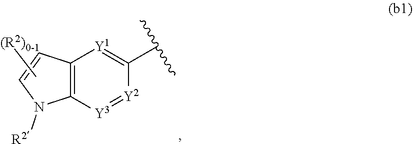

wherein R^{2′} is H or R² (e.g., R^{2′} is H).

301. The compound of clause 300, wherein the

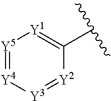

moiety is

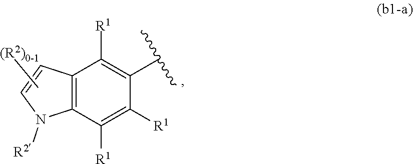

wherein R^{2′} is H or R².

302. The compound of clause 300, wherein the

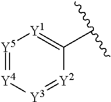

moiety is

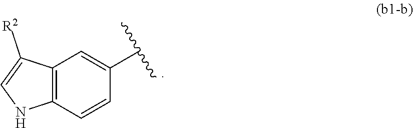

303. The compound of clause 300, wherein the

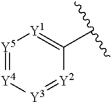

moiety is

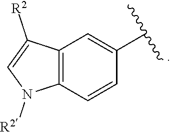

304. The compound of clause 300, wherein the

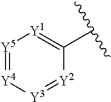

moiety is

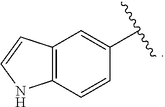 [SMILES: *c1ccc2[nH]ccc2c1]

305. The compound of any one of clauses 238-292, wherein the

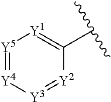

moiety is

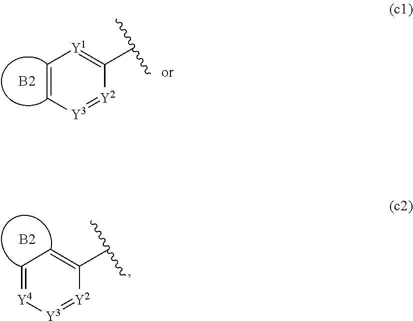

wherein B2 is an aromatic ring including 5 ring atoms, wherein from 1-2 (e.g., 2) ring atoms are heteroatoms each independently selected from the group consisting of N, N(H), N(R^{d}), O, and S(O)₀₋₂, provided that B2 is other than pyrrolyl; and wherein the ring is optionally substituted with from 1-4 independently selected R².

306. The compound of clause 305, wherein B2 is

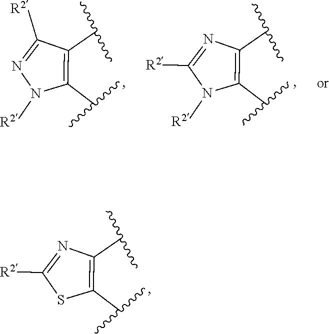

wherein each R^{2′} is independently H or R² (e.g.,

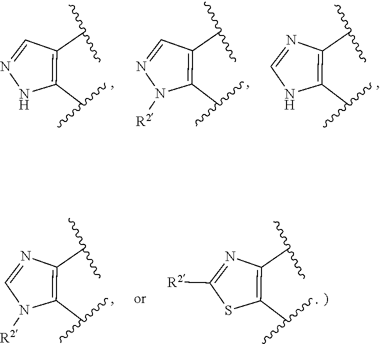

307. The compound of any one of clauses 238-292, wherein the

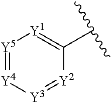

moiety is

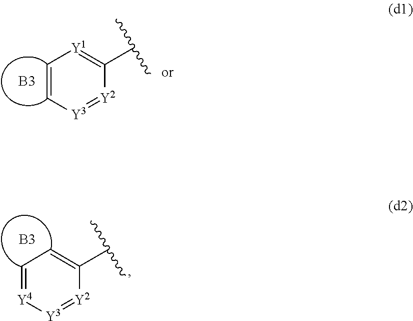

wherein B3 is selected from the group consisting of:

a) a non-aromatic ring including from 5-6 ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

b) a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

308. The compound of clause 307, wherein B3 is a non-aromatic ring including 5 ring atoms, wherein from 1-2 ring atoms are heteroatoms each independently selected from the group consisting of N, N(H), N(R^{d}), O, and S(O)₀₋₂; wherein the ring is substituted with from 1-2 oxo groups; and wherein the ring is further optionally substituted with from 1-2 independently selected R² (e.g.,

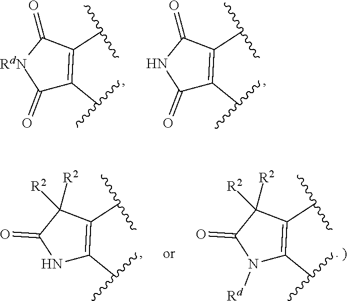

309. The compound of clause 307, wherein B3 is non-aromatic ring including 5 ring atoms, wherein from 0-1 ring atoms is a heteroatom selected from the group consisting of N, N(H), N(R^{d}), O, and S(O)₀₋₂; wherein the ring is optionally substituted with from 1-2 independently selected R² (e.g.,

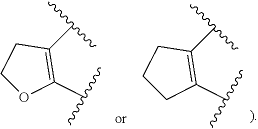 [SMILES: *C1=C(*)OCC1 ; *C1=C(*)CCC1]

310. The compound of clause 307, wherein B3 is a ring (e.g., a spirocyclic ring) including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R².

311. The compound of clause 310, wherein B3 is a spirocyclic bicyclic ring including from 8-12 (e.g., 9-12) ring atoms, wherein from 0-2 ring atoms are heteroatoms each independently selected from the group consisting of N, N(H), N(R^{d}), O, and S(O)₀₋₂; and wherein the ring is optionally substituted with from 1-4 independently selected R² (e.g., B3 is

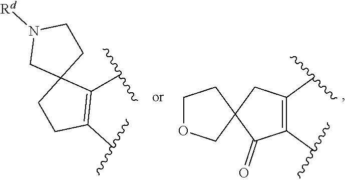

each of which is further optionally substituted with from 1-2 independently selected R²).

312. The compound of any one of clauses 238-292, wherein the

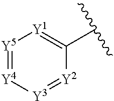

moiety is

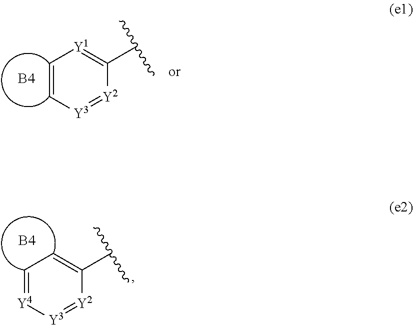

wherein B4 is an aromatic ring including 6 ring atoms, wherein from 0-2 ring atoms are heteroatoms each independently selected from the group consisting of N, N(H), and N(R^{d}); and wherein the ring is optionally substituted with from 1-4 independently selected R².

313. The compound of any one of clauses 238-292, wherein when the

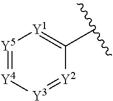

moiety is (aa1), (a1), (b1), (c1), (d1), or (e1), each of Y¹, Y², and Y³ is an independently selected CR¹; and

when the

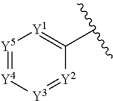

moiety is (aa2), (a2), (b2), (c2), (d2), or (e2), each of Y², Y³, and Y⁴ is an independently selected CR¹.

314. The compound of anyone of clauses 238-292, wherein when the

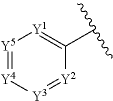

moiety is (aa1), (a1), (b1), (c1), (dl), or (el), one of Y¹, Y², and Y³ is N; and each of the remaining of Y¹, Y², and Y³ is an independently selected CR¹; and

when the

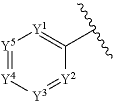

moiety is (aa2), (a2), (b2), (c2), (d2), or (e2), one of Y², Y³, and Y⁴ is N; and each of the remaining of Y², Y³, and Y⁴ is an independently selected CR¹.

315. The compound of any one of clauses 238-292, wherein the

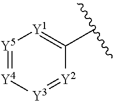

moiety is selected from the group consisting of:

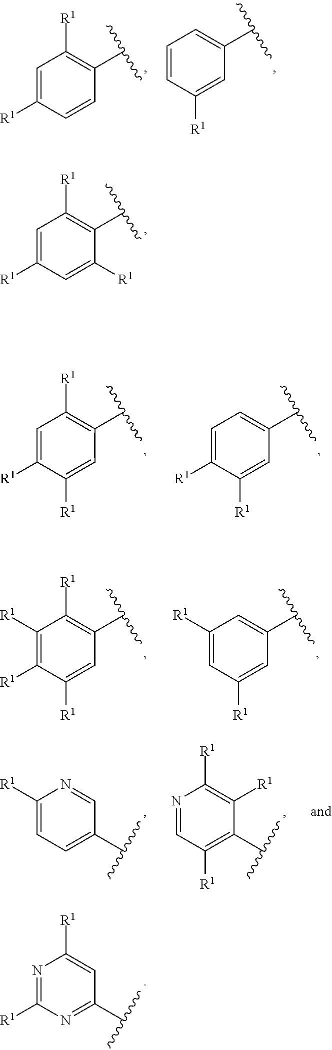

316. The compound of any one of clauses 238-315, wherein each occurrence of R¹ is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —NR^{e}R^{f}; —OH; oxo; —S(O)₁₋₂(NR′R″); —C(═O)(C₁₋₄ alkyl); —C(═O)O(C₁₋₄ alkyl); —C(═O)OH; —C(═O)N(R′)(R″); and -L³-L⁴-R^{a}.

317. The compound of any one of clauses 238-316, wherein R¹ is as defined in any one of clauses 67-75 (e.g., clause 67, 68, 69, 70, 71, 72, 73, 74, or 75).

318. The compound of any one of clauses 238-316, wherein one occurrence of R¹ that is not taken together with the atom to which it is attached in ring formation is selected from the consisting of: halo, cyano, —C(═O)O(C₁₋₄ alkyl), —C(═O)OH, and C₁₋₆ alkyl optionally substituted with 1-2 R^{a}; and each remaining R¹ that is not taken together with the atom to which it is attached in ring formation is H.

319. The compound of any one of clauses 238-316, wherein one occurrence of R¹ that is not taken together with the atom to which it is attached in ring formation is —Rⁱ; and each remaining R¹ that is not taken together with the atom to which it is attached in ring formation is H.

320. The compound of any one of clauses 238-316, wherein each R¹ is H.

321. The compound of clause 238-316, wherein from 1-2 occurrences of R¹ is other than H.

322. The compound of any one of clauses 238-321, wherein each occurrence of R² is as defined in any one of clauses 76-97 (e.g., clause 76 or clause 77 (e.g., R² is halo such as —F or —Cl )).

323. The compound of clause 322, wherein each occurrence of R² is independently selected from the group consisting of halo, cyano, —C(═O)O(C₁₋₄ alkyl), —C(═O)OH, and C₁₋₆ alkyl optionally substituted with 1-2 R^{a}.

324. The compound of any one of clauses 1-323, wherein R^{a} is H.

325. The compound of any one of clauses 1-323, wherein R^{a} is C₁₋₃ alkyl.

326. The compound of any one of clauses 1-325, wherein each occurrence of R^{N} is independently H or C₁₋₃ alkyl.

327. The compound of any one of clauses 1-326, wherein each occurrence of R^{N} is independently H.

328. The compound of clause 1, wherein the compound is selected from the group consisting of the compound delineated in Table C1 or a pharmaceutically acceptable salt thereof.

329. A pharmaceutical composition comprising a compound of clauses 1-328 or a pharmaceutically acceptable salt thereof, and one or more pharmaceutically acceptable excipients.

330. A method for inhibiting STING activity, the method comprising contacting STING with a compound as described in any one of clauses 1-328, or a pharmaceutically acceptable salt thereof, or a pharmaceutical composition as described in clause 329.

331. The method of clause 330, wherein the inhibiting comprises antagonizing STING.

332. The method of any one of clauses 330-331, which is carried out in vitro.

333. The method of clause 332, wherein the method comprises contacting a sample comprising one or more cells comprising STING with the compound.

334. The method of clause 332 or 333, wherein the one or more cells are one or more cancer cells.

335. The method of clause 333 or 334, wherein the sample further comprises one or more cancer cells (e.g., wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma).

336. The method of clause 330, which is carried out in vivo.

337. The method of clause 336, wherein the method comprises administering the compound to a subject having a disease in which increased (e.g., excessive) STING signaling contributes to the pathology and/or symptoms and/or progression of the disease.

338. The method of clause 337, wherein the subject is a human.

339. The method of clause 337, wherein the disease is cancer.

340. The method of clause 339, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

341. The method of clause 339 or 340, wherein the cancer is a refractory cancer.

342. The method of clause 337, wherein the compound is administered in combination with one or more additional cancer therapies.

343. The method of clause 342, wherein the one or more additional cancer therapies comprises surgery, radiotherapy, chemotherapy, toxin therapy, immunotherapy, cryotherapy or gene therapy, or a combination thereof.

344. The method of clause 343, wherein chemotherapy comprises administering one or more additional chemotherapeutic agents.

345. The method of clause 344, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacilitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155 (e.g., CTLA-4 or PD1 or PD-L1).

346. The method of any one of clauses 337-345, wherein the compound is administered intratumorally.

347. A method of treating cancer, comprising administering to a subject in need of such treatment an effective amount of a compound as described in any one of clauses 1-328, or a pharmaceutical composition as described in clause 329.

348. The method of clause 347, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

349. The method of clause 347 or 348, wherein the cancer is a refractory cancer.

350. The method of clause 347, wherein the compound is administered in combination with one or more additional cancer therapies.

351. The method of clause 350, wherein the one or more additional cancer therapies comprises surgery, radiotherapy, chemotherapy, toxin therapy, immunotherapy, cryotherapy or gene therapy, or a combination thereof.

352. The method of clause 351, wherein chemotherapy comprises administering one or more additional chemotherapeutic agents.

353. The method of clause 352, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155 (e.g., CTLA-4 or PD1 or PD-L1). 354. The method of any one of clauses 347-353, wherein the compound is administered intratumorally.

355. A method of inducing an immune response in a subject in need thereof, the method comprising administering to the subject an effective amount of a compound as described in any one of clauses 1-328, or a pharmaceutical composition as described in clause 329.

356. The method of clause 355, wherein the subject has cancer.

357. The method of clause 356, wherein the subject has undergone and/or is undergoing and/or will undergo one or more cancer therapies.

358. The method of clause 356, wherein the cancer selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

359. The method of clause 358, wherein the cancer is a refractory cancer.

360. The method of clause 355, wherein the immune response is an innate immune response.

361. The method of clause 360, wherein the at least one or more cancer therapies comprises surgery, radiotherapy, chemotherapy, toxin therapy, immunotherapy, cryotherapy or gene therapy, or a combination thereof.

362. The method of clause 361, wherein chemotherapy comprises administering one or more additional chemotherapeutic agents.

363. The method of clause 362, wherein the one or more additional chemotherapeutic agents is selected from alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacllitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155 (e.g., CTLA-4 or PD1 or PD-L1). 364. A method of treatment of a disease in which increased (e.g., excessive) STING signaling contributes to the pathology and/or symptoms and/or progression of the disease, comprising administering to a subject in need of such treatment an effective amount of a compound as described in any one of clauses 1-328, or a pharmaceutical composition as described in clause 329.

365. A method of treatment comprising administering to a subject having a disease in which increased (e.g., excessive) STING signaling contributes to the pathology and/or symptoms and/or progression of the disease an effective amount of a compound as described in any one of clauses 1-328, or a pharmaceutical composition as described in clause 329.

366. A method of treatment comprising administering to a subject a compound as described in any one of clauses 1-328, or a pharmaceutical composition as described in clause 329, wherein the compound or composition is administered in an amount effective to treat a disease in which increased (e.g., excessive) STING signaling contributes to the pathology and/or symptoms and/or progression of the disease, thereby treating the disease.

367. The method of any one of clauses 364-366, wherein the disease is cancer.

368. The method of clause 367, wherein the cancer is selected from the group consisting of melanoma, cervical cancer, breast cancer, ovarian cancer, prostate cancer, testicular cancer, urothelial carcinoma, bladder cancer, non-small cell lung cancer, small cell lung cancer, sarcoma, colorectal adenocarcinoma, gastrointestinal stromal tumors, gastroesophageal carcinoma, colorectal cancer, pancreatic cancer, kidney cancer, hepatocellular cancer, malignant mesothelioma, leukemia, lymphoma, myelodysplasia syndrome, multiple myeloma, transitional cell carcinoma, neuroblastoma, plasma cell neoplasms, Wilm's tumor, or hepatocellular carcinoma.

369. The method of clause 367 or 368, wherein the cancer is a refractory cancer.

370. The method of any one of clauses 367-369, wherein the compound is administered in combination with one or more additional cancer therapies.

371. The method of clause 370, wherein the one or more additional cancer therapies comprises surgery, radiotherapy, chemotherapy, toxin therapy, immunotherapy, cryotherapy or gene therapy, or a combination thereof.

372. The method of clause 371, wherein chemotherapy comprises administering one or more additional chemotherapeutic agents.

373. The method of clause 372, wherein the one or more additional chemotherapeutic agents is selected from an alkylating agent (e.g., cisplatin, carboplatin, mechlorethamine, cyclophosphamide, chlorambucil, ifosfamide and/or oxaliplatin); an anti-metabolite (e.g., azathioprine and/or mercaptopurine); a terpenoid (e.g., a vinca alkaloid and/or a taxane; e.g., Vincristine, Vinblastine, Vinorelbine and/or Vindesine Taxol, Pacilitaxel and/or Docetaxel); a topoisomerase (e.g., a type I topoisomerase and/or a type 2 topoisomerase; e.g., camptothecins, such as irinotecan and/or topotecan; amsacrine, etoposide, etoposide phosphate and/or teniposide); a cytotoxic antibiotic (e.g., actinomycin, anthracyclines, doxorubicin, daunorubicin, valrubicin, idarubicin, epirubicin, bleomycin, plicamycin and/or mitomycin); a hormone (e.g., a lutenizing hormone releasing hormone agonist; e.g., leuprolidine, goserelin, triptorelin, histrelin, bicalutamide, flutamide and/or nilutamide); an antibody (e.g., Abciximab, Adalimumab, Alemtuzumab, Atlizumab, Basiliximab, Belimumab, Bevacizumab, Bretuximab vedotin, Canakinumab, Cetuximab, Ceertolizumab pegol, Daclizumab, Denosumab, Eculizumab, Efalizumab, Gemtuzumab, Golimumab, Golimumab, Ibritumomab tiuxetan, Infliximab, Ipilimumab, Muromonab-CD3, Natalizumab, Ofatumumab, Omalizumab, Palivizumab, Panitumuab, Ranibizumab, Rituximab, Tocilizumab, Tositumomab and/or Trastuzumab); an anti-angiogenic agent; a cytokine; a thrombotic agent; a growth inhibitory agent; an anti-helminthic agent; and an immune checkpoint inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9-TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TL1A, CD40L, CD40-CD40 ligand, HVEM-LIGHT-LTA, HVEM, HVEM—BTLA, HVEM—CD160, HVEM—LIGHT, HVEM-BTLA-CD160, CD80, CD80-PDL-1, PDL2-CD80, CD244, CD48-CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86-CD28, CD86-CTLA, CD80-CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155 (e.g., CTLA-4 or PD1 or PD-L1).

374. The method of any one of clauses 364-373, wherein the compound is administered intratumorally.

375. A method of treatment of a disease, disorder, or condition associated with STING, comprising administering to a subject in need of such treatment an effective amount of a compound as described in any one of clauses 1-328, or a pharmaceutical composition as described in clause 329.

376. The method of clause 375, wherein the disease, disorder, or condition is selected from type I interferonopathies, Aicardi-Goutières Syndrome (AGS), genetic forms of lupus, inflammation-associated disorders, and rheumatoid arthritis.

377. The method of clause 376, wherein the disease, disorder, or condition is a type I interferonopathy (e.g., STING-associated vasculopathy with onset in infancy (SAVI)).

378. The method of clause 377, wherein the type I interferonopathy is STING-associated vasculopathy with onset in infancy (SAVI)).

379. The method of clause 376, wherein the disease, disorder, or condition is Aicardi-Goutières Syndrome (AGS).

380. The method of clause 376, wherein the disease, disorder, or condition is a genetic form of lupus.

381. The method of clause 376, wherein the disease, disorder, or condition is inflammation-associated disorder.

382. The method of clause 381, wherein the inflammation-associated disorder is systemic lupus erythematosus.

383. The method of any one of clauses 330-382, wherein the method further comprises identifying the subject.

What is claimed is:

1. A compound of Formula I: 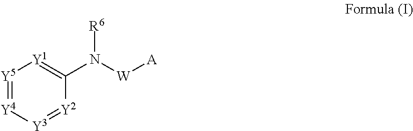
or a pharmaceutically acceptable salt thereof or a tautomer thereof,
wherein:
each of Y¹, Y², Y³, Y⁴, and Y⁵ is independently selected from the group consisting of N and CR¹;
W-A is defined according to (A) or (B) below:
(A)
W is selected from the group consisting of:
(k) *C(═O)NR^{N}, *C(═S)NR^{N}, *C(═NR^{N})NR^{N} (e.g., *C(═NCN)NR^{N}), *C(═CNO₂)NR^{N}
(l) *S(O)₁₋₂NR^{N}; 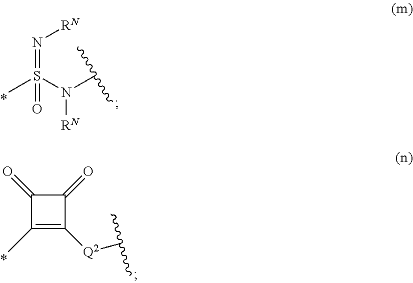
(o) *Q¹-Q²;
wherein the asterisk denotes point of attachment to NR⁶;
Q¹ is selected from the group consisting of:
(e) phenylene optionally substituted with from 1-2 independently selected R^{q1}; and
(f) heteroarylene including from 5-6 ring atoms, wherein from 1-4 ring atoms are heteroatoms, each independently selected from the group consisting of N, N(H), N(R^{d}), O, and S(O)₀₋₂, and wherein the heteroarylene ring is optionally substituted with from 1-4 independently selected R^{q1};
Q² is selected from the group consisting of: a bond, NR^{N}, —S(O)₀₋₂—, —O—, and —C(═O)—;
A is:
(i) —Y^{A1}-Y^{A2}, wherein:
Y^{A1} is a bond; or
Y^{A1} is C₁₋₆ alkylene, which is optionally substituted with from 1-6 substituents each independently selected from the group consisting of:
R^{a};
C₆₋₁₀ aryl optionally substituted with 1-4 independently selected C₁₋₄ alkyl; and
heteroaryl including from 5-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected C₁₋₄ alkyl; or
Y^{A1} is —Y^{A3}-Y^{A4}—Y^{A5} which is connected to W via Y^{A3} wherein:
Y^{A3} is a C₁₋₃ alkylene optionally substituted with from 1-2 independently selected R^{a};
Y^{A4} is —O—, —NH—, or —S—; and
Y^{A5} is a bond or C₁₋₃ alkylene which is optionally substituted with from 1-2 independently selected R^{a}; and
Y^{A2} is:
(a) C₃₋₂₀ cycloalkyl, which is optionally substituted with from 1-4 R^{b},
(b) C₆₋₂₀ aryl, which is optionally substituted with from 1-4 R^{c};
(c) heteroaryl including from 5-20 ring atoms, wherein from 1-3 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; or
(d) heterocyclyl including from 3-16 ring atoms, wherein from 1-3 ring atoms are heteroatoms, each independently selected from the group consisting of N, N(H), N(R^{d}), O, and S(O)₀₋₂, and wherein the heterocyclyl ring is optionally substituted with from 1-4 independently selected R^{b},
OR
(ii) —Z¹-Z²-Z³, wherein:
Z¹ is C₁₋₃ alkylene, which is optionally substituted with from 1-4 R^{a};
Z² is —N(H)—, —N(R^{d})—, —O—, or —S—; and
Z³ is C₂₋₇ alkyl, which is optionally substituted with from 1-4 R^{a};
OR
(iii) C₁₋₂₀ alkyl, which is optionally substituted with from 1-6 independently selected R^{a},
OR
(B)
W is selected from the group consisting of:
(a) C₈₋₂₀ bicyclic or polycyclic arylene, which is optionally substituted with from 1-4 R^{c}; and
(b) bicyclic or polycyclic heteroarylene including from 8-20 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c};
A is as defined for A or A is H;
each occurrence of R¹ is independently selected from the group consisting of
H;
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl),
—S(O)(═NH)(C₁₋₄ alkyl),
SF₅
—NR^{e}R^{f},
—OH,
oxo,
—S(O)₁₋₂(NR′R″),
—C₁₋₄ thioalkoxy,
—NO₂,
—C(═O)(C₁₋₄ alkyl),
—C(═O)O(C₁₋₄ alkyl),
—C(═O)OH,
—C(═O)N(R′)(R″), and
—L³-L⁴-L⁵-Rⁱ;
or a pair of R¹ on adjacent atoms, taken together with the atoms connecting them, form a ring (e.g., aromatic or non-aromatic ring) including from 4-15 ring atoms, wherein from 0-3 ring atoms are heteroatoms each independently selected from the group consisting of N, N(H), N(R^{d}), O, and S(O)₀₋₂; and wherein the ring is optionally substituted with from 1-4 independently selected R²;
each R² is independently selected from the group consisting of:
halo;
cyano;
C₁₋₆ alkyl optionally substituted with 1-2 R^{a};
C₂₋₆ alkenyl;
C₂₋₆ alkynyl;
C₁₋₄ haloalkyl;
C₁₋₄ alkoxy;
C₁₋₄ haloalkoxy;
—S(O)₁₋₂(C₁₋₄ alkyl) optionally substituted with from 1-3 independently selected R^{a},
—S(O)(═NH)(C₁₋₄ alkyl) optionally substituted with from 1-3 independently selected R^{a},
SF₅,
—NR^{e}R^{f},
—OH,
oxo,
—S(O)₁₋₂(NR′R″),
—C₁₋₄ thioalkoxy,
—NO₂,
—C(═O)(C₁₋₄ alkyl) optionally substituted with from 1-3 independently selected R^{a},
—C(═O)O(C₁₋₄ alkyl) optionally substituted with from 1-3 independently selected R^{a},
—C(═O)OH,
—C(═O)N(R′)(R″); and
—L³-L⁴-L⁵-Rⁱ;
R⁶ is selected from H; C₁₋₆ alkyl; —OH; C₁₋₄ alkoxy; C(═O)H; C(═O)(C₁₋₄ alkyl); CN; C₆₋₁₀ aryl optionally substituted with from 1-4 independently selected C₁₋₄ alkyl; and heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 independently selected C₁₋₄ alkyl;
each occurrence of R^{q1} is independently selected from the group consisting of:
(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (f) C₃₋₆ cycloalkyl; (g) C₁₋₄ alkoxy; (h) C₁₋₄ haloalkoxy; (i) —S(O)₁₋₂(C₁₋₄ alkyl); (j) —NR^{e}R^{f}; (k) —OH; (l) —S(O)₁₋₂(NR′R″); (m) —C₁₋₄ thioalkoxy; (n) —NO₂; (o) —C(═O)(C₁₋₄ alkyl); (p) —C(═O)O(C₁₋₄ alkyl); (q) —C(═O)OH; (r) —C(═O)N(R′)(R″); and (s) oxo;
each occurrence of R^{a} is independently selected from the group consisting of: —OH; —F; —Cl ; —Br; —NR^{e}R^{f}; C₁₋₄ alkoxy; C₁₋₄ haloalkoxy; —C(═O)O(C₁₋₄ alkyl); —C(═O)(C₁₋₄ alkyl); —C(═O)OH; —CON(R′)(R″); —OCON(R′)(R″); —S(O)₁₋₂(NR′R″); —S(O)₁₋₂(C₁₋₄ alkyl); cyano, and C₃₋₆ cycloalkyl optionally substituted with from 1-4 independently selected C₁₋₄ alkyl;
each occurrence of R^{b} is independently selected from the group consisting of: C₁₋₁₀ alkyl optionally substituted with from 1-6 independently selected R^{a}; C₁₋₄ haloalkyl; —OH; oxo; —F; —Cl ; —Br; —NR^{e}R^{f}; C₁₋₄ alkoxy; C₁₋₄ haloalkoxy; —C(═O)(C₁₋₁₀ alkyl); —C(═O)O(C₁₋₄ alkyl); —C(═O)OH; —C(═O)N(R′)(R″); —S(O)₁₋₂(NR′R″); —S(O)₁₋₂(C₁₋₄ alkyl); cyano; and -L¹-L²-R^{h};
each occurrence of R^{c} is independently selected from the group consisting of:
(a) halo; (b) cyano; (c) C₁₋₁₀ alkyl which is optionally substituted with from 1-6 independently selected R^{a}; (d) C₂₋₆ alkenyl; (e) C₂₋₆ alkynyl; (g) C₁₋₄ alkoxy; (h) C₁₋₄ haloalkoxy; (i) —S(O)₁₋₂(C₁₋₄ alkyl) or —S(O)₁₋₂(C₁₋₄ haloalkyl); (j) —NR^{e}R^{f}; (k) —OH; (l) —S(O)₁₋₂(NR′R″); (m) —C₁₋₄ thioalkoxy or —C₁₋₄ thiohaloalkoxy; (n) —NO₂; (o) —C(═O)(C₁₋₁₀ alkyl); (p) —C(═O)O(C₁₋₄ alkyl); (q) —C(═O)OH; (r) —C(═O)N(R′)(R″); (s) -L¹-L²-R^{h}; (t) —SF₅; and (u) azido;
each occurrence of R^{d} is selected from the group consisting of: C₁₋₆ alkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —OH; C₁₋₄ alkoxy; and CN;
each occurrence of R^{e} and R^{f} is independently selected from the group consisting of: H; C₁₋₆ alkyl, wherein the C₁₋₆ alkyl is independently selected with from 1-4 substituents each independently selected from halo, CN, C₁₋₄ alkoxy, C₁₋₄ haloalkoxy, NR′R″, and —OH; C₁₋₆ haloalkyl; C₃₋₆ cycloalkyl; —C(O)(C₁₋₄ alkyl); —C(O)O(C₁₋₄ alkyl); —CON(R′)(R″); —S(O)₁₋₂(NR′R″); —S(O)₁₋₂(C₁₋₄ alkyl); —S(O)(═NR′)(C₁₋₄ alkyl); —OH; and C₁₋₄ alkoxy; or
R^{e} and R^{f} together with the nitrogen atom to which each is attached forms a ring including from 3-8 ring atoms, wherein the ring includes: (a) from 1-7 ring carbon atoms, each of which is substituted with from 1-2 substituents independently selected from H and C₁₋₃ alkyl; and (b) from 0-3 ring heteroatoms (in addition to the nitrogen atom attached to R^{e} and R^{f}), which are each independently selected from the group consisting of N(R^{d}), NH, O, and S;
—L¹ is a bond or C₁₋₃ alkylene optionally substituted with oxo;
—L² is —O—, —N(H)—, —S(O)₀₋₂—, or a bond;
R^{h} is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R^{h} is C₃₋₆ cycloalkyl optionally substituted with from 1-4 independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
—L¹ is a bond or C₁₋₃ alkylene optionally substituted with oxo;
—L⁴ is a bond; —O—; —N(R^{N})—; —S(O)₀₋₂—; C(═O); —NR^{N}S(O)₀₋₂—; —S(O)₀₋₂NR^{N}—; —NR^{N}S(O)₁₋₂NR^{N}—; —S(═O)(═NR^{N}); —NR^{N}S(═O)(═NR^{N}); —S(═O)(═NR^{N})NR^{N}; NR^{N}S(═O)(═NR^{N})NR^{N};
—NR^{N}C(O)—; —NR^{N}C(O)NR^{N}—; C₃₋₆ cycloalkylene; or heterocyclylene including from 3-8 ring atoms wherein from 1-3 ring atoms are heteroatoms each independently selected from the group consisting of N, NH, N(R^{d}), O, and S(O)₀₋₂;
—L⁵ is a bond or C₁₋₄ alkylene;
Rⁱ is selected from:
C₃₋₈ cycloalkyl optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy (in certain embodiments, it is provided that when R¹ is C₃₋₆ cycloalkyl optionally substituted with from 1-4 substituents independently selected C₁₋₄ alkyl, -L¹ is a bond, or -L² is —O—, —N(H)—, or —S—);
heterocyclyl, wherein the heterocyclyl includes from 3-16 ring atoms, wherein from 1-3 ring atoms are heteroatoms, each independently selected from the group consisting of N, N(H), N(R^{d}), O, and S(O)₀₋₂, wherein the heterocyclyl is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
heteroaryl including from 5-10 ring atoms, wherein from 1-4 ring atoms are heteroatoms, each independently selected from the group consisting of N, N(H), N(R^{d}), O, and S(O)₀₋₂ and wherein the heteroaryl ring is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy; and
C₆₋₁₀ aryl, which is optionally substituted with from 1-4 substituents independently selected from the group consisting of halo; C₁₋₄ alkyl optionally substituted with from 1-2 independently selected R^{a}; C₁₋₄ haloalkyl; cyano; C₁₋₄ alkoxy; and C₁₋₄ haloalkoxy;
each occurrence of R^{N} is independently H or R^{d}; and
each occurrence of R′ and R″ is independently selected from the group consisting of: H, C₁₋₄ alkyl, and C₆₋₁₀ aryl optionally substituted with from 1-2 substituents selected from halo, C₁₋₄ alkyl, and C₁₋₄ haloalkyl; or R′ and R″ together with the nitrogen atom to which each is attached forms a ring including from 3-8 ring atoms, wherein the ring includes: (a) from 1-7 ring carbon atoms, each of which is substituted with from 1-2 substituents independently selected from the group consisting of H and C₁₋₃ alkyl; and (b) from 0-3 ring heteroatoms (in addition to the nitrogen atom attached to R′ and R″), which are each independently selected from the group consisting of N(H), N(C₁₋₆alkyl), O, and S;
provided that when the compound has Formula (I-a1) wherein R^{2′} is H or R², W-A is defined according to (A), and W is *C(O)NR^{N} (e.g., *C(O)NH), then 1, 2, 3, 4, or 5 of the following provisions apply: 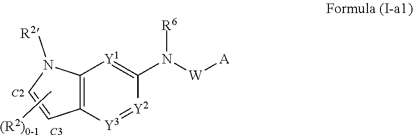
(i) when each of Y¹ and Y² is CH; Y³ is CR¹; R¹ is CO₂Me, CO₂Et, CN, or Cl ; and
R² is absent (i.e., C2 and C3 are substituted with H), OR when each of Y¹ and Y² is N; and
Y³ is OH or oxo, then A cannot be optionally substituted C₁₋₆ alkyl, such as methyl or butyl, 1,1,3,3-tetramethylbutyl, or optionally substituted C₃ or C₆ cycloalkyl (such as C₁₋₆ alkyl or C₃ or C₆ cycloalkyl optionally substituted with CO₂H, isocyanate, or substituted amino);
(ii) when each of Y¹ and Y² is N; and Y³ is CR¹; then
R¹ cannot be furyl, when W-A is benzyl; and
R¹ cannot be substituted N-linked aniline or chloro when either R^{2′} is methyl or when W-A is phenyl substituted with from 1-2 substituents independently selected from —Cl , —F, —Br, and CF₃;
(iii) when each of Y¹, Y², and; Y³ is CH; R^{2′} is H, R² is present and attached at the C3-position of the indole ring; and A is phenyl, tolyl, optionally substituted quinazolinyl, optionally substituted pyrazolyl, optionally substituted indolyl, optionally substituted naphthyl, or optionally substituted moropholinyl-phenyl, then R² cannot be oxazolyl, pyridyl, C-linked-2-pyridylethyl, phenyl, cyano, or C(O)NH₂;
(iv) when each of Y¹ and Y³ is CH; Y² is CH or CMe; R^{2′} is H; and R² is absent, then:
R^{h} cannot be a fused tricyclic ring;
Y^{A2} cannot be optionally substituted cyclohexyl, cyclohexenyl, imidazo[1,2-a][1,4]benzodiazepin-4-yl, indenyl, naphthyl, or tetrahydronaphthyl;
Y^{A1} cannot be alkylene substituted with phenyl;
when Y^{A1} is alkylene, Y^{A2} cannot be phenyl or the following substituted phenyl rings: 4-Br, 2,4-(Cl)₂, 3-propenyl, 2,3-(OMe)₂, and 4-CF₃; and
when Y^{A1} is absent, Y^{A2} cannot be phenyl or the following substituted phenyl rings: 3-NO₂, 4-Br, 2,4-(Cl)₂, 2,3-(OMe)₂, 4-CF₃, 4-CO₂Et, 3-CF₃-4-Cl, 2-Cl-4 CF₃, 2-OEt, 2-OMe-4-NO₂, 3,4-(OMe)₂, 2,4-(Me)₂, 3,4-(Cl )₂, 2,4-(F)₂, 2-Et, 2-F, 2-Me, 2-Br, 2-Cl-4-Br, 2-CF₃, 2,4-(OMe)₂, 2,3-(Me)₂, 3,5-(Cl )₂, 3-CF₃-4-F, 4-iso-propyl, 4-OMe, 4-Cl, 3-F-4-Me, 3-CF₃, 2,5-(OMe)₂, 2-Me-3-Cl, 2,3-(Me)₂, 2,3-(Cl )₂, 4-Bu, 3-OMe, 3-Cl, 4-Me-2-Cl, 3-SMe, 2-CO₂Me, 4-Me-3-Cl, 3,4-(Me)₂, 4-sec-butyl, 2-OMe, 2-Cl, 2,4-(OMe)₂-5-Cl, 4-OEt, 4-acetyl, 2-OMe-5-Me, 2-Me-5-Cl, 3,5-(Me)₂, 3,5-(Cl )₂, 4-NO₂, 4-Br, 4-F, 4-Me, 4-Et, 3-F, 3-Me, 3-acetyl, or 2-Me-5-Cl ; and
(v) the compound is other than:

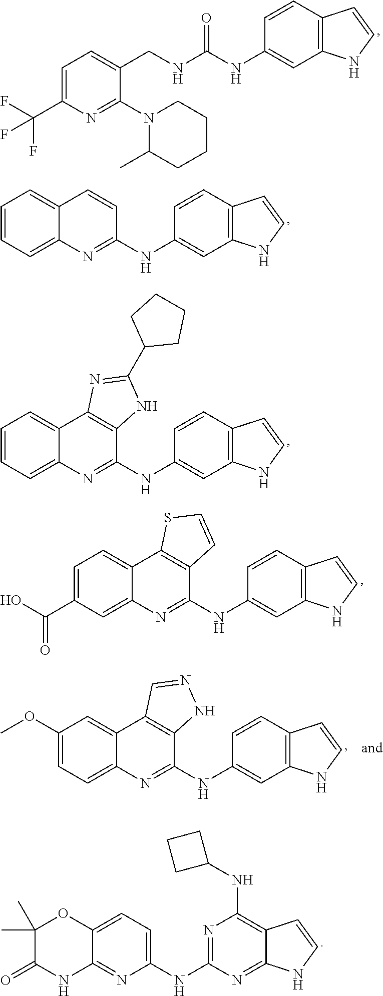

2. The compound of claim 1, wherein a pair of R¹ on adjacent atoms, taken together with the atoms connecting them, form an aromatic ring including 5 ring atoms, wherein from 1-2 (such as 1 or 2) ring atoms are heteroatoms each independently selected from the group consisting of N, N(H), N(R^{d}), O, and S(O)₀₋₂; and wherein the ring is optionally substituted with from 1-4 independently selected R².

3. The compound of claims 1 or 2, wherein a pair of R¹ on adjacent atoms, taken together with the atoms connecting them, form: 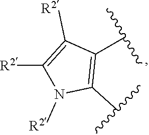
wherein each R^{2′} is independently H or R², such as 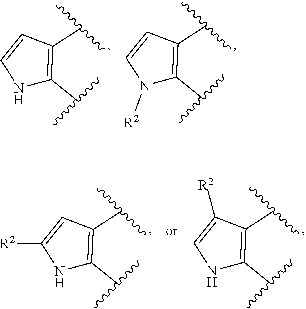
such as

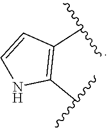 [SMILES: *c1cc[nH]c1*]

4. The compound of any one of claims 1-3, wherein the compound has the following formula: 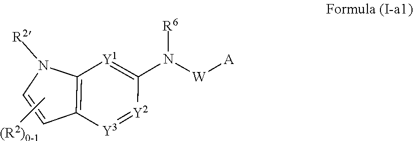
such as, 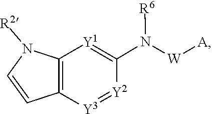
wherein R^{2′} is H or R², such as R^{2′} is H.

5. The compound of any one of claims 1-4, wherein the compound has formula 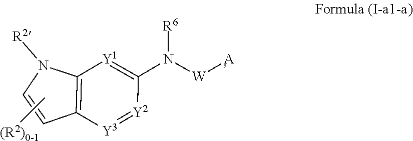
wherein R^{2′} is H or R², such as 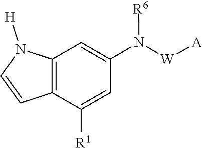
(e.g., R¹ is other than H) Formula (I-a1-b), 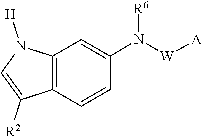
(e.g., R¹ is other than H) Formula (I-a1-e).

6. The compound of any one of claims 1-5, wherein each occurrence of R¹ that is not taken together with the atom to which it is attached in ring formation is independently selected from the group consisting of: H; halo; cyano; C₁₋₆ alkyl optionally substituted with 1-2 R^{a}; C₂₋₆ alkenyl; C₂₋₆ alkynyl; C₁₋₄ haloalkyl; C₁₋₄ alkoxy; C₁₋₄ haloalkoxy; —S(O)₁₋₂(C₁₋₄ alkyl); —NR^{e}R^{f}; —OH; oxo; —S(O)₁₋₂(NR′R″); —C(═O)(C₁₋₄ alkyl); —C(═O)O(C₁₋₄ alkyl); —C(═O)OH; —C(═O)N(R′)(R″); and -L³-L⁴-Rⁱ, such as R¹ is halo; cyano; C₁₋₆ alkyl optionally substituted with 1-2 R^{a}; C₁₋₄ haloalkyl; C₁₋₄ alkoxy; or C₁₋₄ haloalkoxy, such as R¹ is halo.

7. The compound of any one of claims 1-6, wherein W-A as defined according to (A).

8. The compound of claim 7, wherein W is *C(═O)NR^{N}, such as *C(═O)NH.

9. The compound of any one of claims 1-6, wherein W-A is as defined according to (B).

10. The compound of any one of claims 1-6 and 9, wherein W is bicyclic heteroarylene including from 8-10 ring atoms, wherein from 1-4 ring atoms are heteroatoms, each independently selected from the group consisting of N, N(H), N(R^{d}), O, and S(O)₀₋₂, and wherein the heteroaryl ring is optionally substituted with from 1-4 independently selected R^{c}; and A is H,
such as W is selected from the group consisting of quinolinylene, isoquinolinylene, and quinazolinylene, each of which is optionally substituted with from 1-2 independently selected R^{c}, such as W is

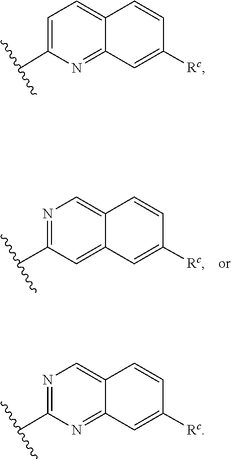

11. The compound of any one of claims 1-9, wherein A is —Y^{A1}-Y^{A2}.

12. The compound of any one of claims 1-9 or 11, wherein Y^{A2} is C₆₋₁₀ aryl, which is optionally substituted with from 1-3 R^{c}.

13. The compound of claim 1, wherein the compound has one of the following formulae: 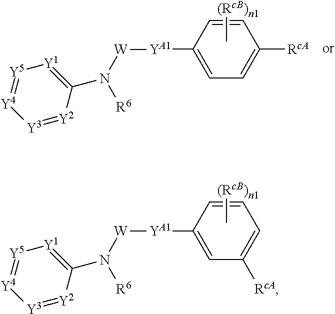
wherein:
n1 is 0, 1, or 2 (such as 0 or 1); each of R^{cA} and R^{cB} is an independently selected R^{c};
W is *C(═O)NR^{N}, such as *C(═O)NH; and
the 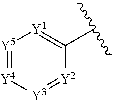
moiety is 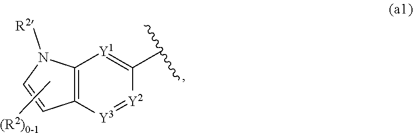
wherein R^{2′} is H or R².

14. The compound of claim 13, wherein the 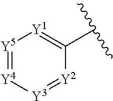
moiety is 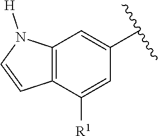
(a1-b), such as (a1-b) wherein R¹ is other than H (e.g., R¹ is halo or cyano).

15. The compound of claim 1, wherein W is heteroarylene including from 9-10 ring atoms, wherein from 1-2 ring atoms are heteroatoms, each independently selected from the group consisting of N, N(H), N(R^{d}), O, and S(O)₀₋₂, and wherein the heteroaryl ring is optionally substituted with from 1-2 independently selected R^{c}, such as
W is selected from the group consisting of quinolinylene and quinazolinylene, each of which is optionally substituted with from 1-2 independently selected R^{c}, such as:
W is 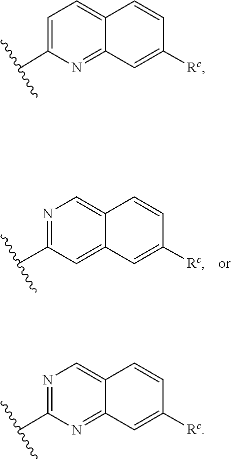
the 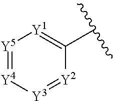
moiety is 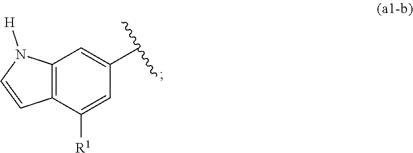
and
A is H, optionally wherein R⁶ is H.

16. The compound of claim 1, wherein the compound is selected from the group consisting of the compounds delineated in Table C1 or a pharmaceutically acceptable salt thereof.

17. A pharmaceutical composition comprising a compound of any one of claims 1-16, or a pharmaceutically acceptable salt thereof, and one or more pharmaceutically acceptable excipients.

18. A method for inhibiting STING activity, the method comprising contacting STING with a compound as claimed in any one of claims 1-16, or a pharmaceutically acceptable salt thereof, or a pharmaceutical composition as claimed in claim 17.

19. A method of inducing an immune response in a subject in need thereof, the method comprising administering to the subject an effective amount of a compound as claimed in any one of claims 1-16, or a pharmaceutically acceptable salt thereof, or a pharmaceutical composition as claimed in claim 17.

20. A method of treatment of disease, disorder, or condition associated with STING, such as a disease, disorder, or condition, in which increased STING signaling, such as excessive STING signaling, contributes to the pathology and/or symptoms and/or progression of the disease, such as cancer, comprising administering to a subject in need of such treatment an effective amount of a compound as claimed in any one of claims 1-16, or a pharmaceutically acceptable salt thereof, or a pharmaceutical composition as claimed in claim 17.
